(19) United States
(12) Patent Application Publication
(10) Pub. No.: US 2025/0034110 A1
(43) Pub. Date: Jan. 30, 2025

(54) LPAR1 ANTAGONISTS AND USES THEREOF

(71) Applicant: Pipeline Therapeutics, Inc., San Diego, CA (US)

(72) Inventors: Jeffrey Roger Roppe, Temecula, CA (US); Austin Chih-Yu Chen, San Diego, CA (US); Yifeng Xiong, San Diego, CA (US); Thomas Schrader, San Diego, CA (US); Lino Valdez, San Diego, CA (US)

(21) Appl. No.: 18/294,515

(22) PCT Filed: Aug. 4, 2022
(86) PCT No.: PCT/US22/39466

§ 371 Date: Feb. 1, 2024

Related U.S. Application Data
(60) Provisional application No. 63/229,858, filed on Aug. 5, 2021.

(51) Int. Cl. C07D 401/12; A61K 31/397; A61K 31/443; A61K 31/4433; A61K 31/4436; A61K 31/4439; A61K 31/444; A61K 31/4545; A61K 31/46; C07D 205/12; C07D 213/75; C07D 401/14; C07D 405/04; C07D 405/12; C07D 405/14; C07D 409/12; C07D 413/12; C07D 419/12; C07D 471/08

(52) U.S. Cl. CPC C07D 401/12; A61K 31/397; A61K 31/443; A61K 31/4433; A61K 31/4436; A61K 31/4439; A61K 31/444; A61K 31/4545; A61K 31/46; C07D 205/12; C07D 213/75; C07D 401/14; C07D 405/04; C07D 405/12; C07D 405/14; C07D 409/12; C07D 413/12; C07D 419/12; C07D 471/08

(57) ABSTRACT

Described herein, inter alia, are LPAR1 antagonists and uses thereof.

### CROSS REFERENCE TO RELATED APPLICATIONS

This application claims priority to U.S. Provisional Application No. 63/229,858, filed Aug. 5, 2021, which is hereby incorporated by reference in its entirety and for all purposes.

### BACKGROUND

Lysophosphatidic acid, or LPA, is a family of bioactive phospholipids that are associated with multiple cellular functions. While the family members differ with regards to the length and the degree of saturation of their respective long-chain fatty acid backbone (Fujiwara et al., J. Biol. Chem., 2005, 280, 35038-35050), they are all capped by a glycerol-phosphate group through an ester linkage. LPAs are produced biologically from membrane phospholipids through a multi-step cascade mediated by enzymes that include lysophospholipase D (lysoPLD), autotaxin (ATX), phospholipase A1 (PLA1), phospholipase A2 (PLA2) and acylglycerol kinase (AGK) (Mutoh et al., British J Pharmacol., 2012, 165, 829-844). Once formed, the LPAs can regulate numerous cellular signaling pathways by binding to a class of 7-membrane domain G protein-coupled receptors (GPCRs), collectively known as LPA receptors (LPARs), of which six have been characterized: LPAR1, LPAR2, LPAR3, LPAR4, LPAR5, and LPAR6 (Choi, J. W., Annu. Rev. Pharmacol. Toxicol., 2010, 50, 157-186). The biological responses elicited by the binding of LPAs to LPARs are both wide-ranging and context-dependent (Yung et al., J. Lipid Res. 2014, 55, 1192-1214; Yung et al., Neuron 2015, 85, 669-682). These can include induction of cell proliferation, stimulation of cell migration and contraction, promotion of neurite retraction, suppression of apoptosis, initiation of chemotaxis, closure of gap junction, and others (Chun et al., Editors, Lysophospholipid Receptors: Signaling and Biochemistry, 2013, Wiley, ISBN: 978-0-470-56905-4). Furthermore, aberrant upregulation of the LPA pathway has been implicated in multiple diseases, including cancer, inflammatory diseases, infertility, neuropathic pain, psychotic and neurodegenerative disorders, atherosclerosis, as well as fibrosis of the skin, kidney, lung, and liver (Choi, J. W., Annu. Rev. Pharmacol. Toxicol., 2010, 50, 157-186; Noguchi et al., Curr. Opin Pharmacol., 2009, 9, 15-23; Yanagida et al., J. Biochem., 2011, 150, 223-232). Consequently, the targeting of LPA receptors has been, and continues to be an area of intense interest for the identification of potential treatments for these disorders. Disclosed herein, inter alia, are solutions to these and other problems in the art.

### BRIEF SUMMARY

In an aspect is provided a compound, or a pharmaceutically acceptable salt or solvate thereof, having the formula:

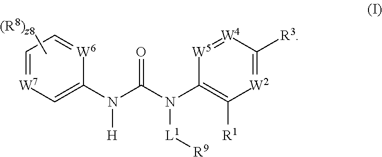

L¹ is a bond or substituted or unsubstituted C₁-C₅ alkylene.

R¹ is unsubstituted C₂-C₅ alkyl.

W² is N or C(R²).

R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

W⁴ is N or C(R⁴).

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

W⁵ is N or C(R⁵).

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² and R³ substituents may optionally be joined to form a substituted or unsubstituted cycloalkyl, substituted or unsubstituted heterocycloalkyl, substituted or unsubstituted aryl, or substituted or unsubstituted heteroaryl.

R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl.

R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl.

W⁶ is N or C(R⁶).

R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

W⁷ is N, N⁺—O⁻, or C(R⁷).

R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl.

R⁹ is substituted or unsubstituted cycloalkyl or substituted or unsubstituted heterocycloalkyl.

R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, and R^{8D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl.

X², X³, X⁴, X⁵, X⁶, X⁷, and X⁸ are independently —F, —Cl, —Br, or —I.

The symbols n2, n3, n4, n5, n6, n7, and n8 are independently an integer from 0 to 4. The symbols m2, m3, m4, m5, m6, m7, m8, v2, v3, v4, v5, v6, v7, and v8 are independently 1 or 2. The symbol z8 is an integer from 0 to 3.

In an aspect is provided a compound, or a pharmaceutically acceptable salt or solvate thereof, having the formula:

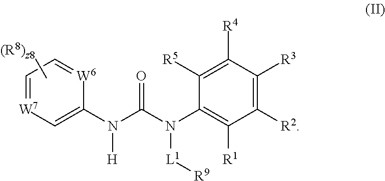

L¹, R², R³, R⁴, R⁵, W⁶, W⁷, R⁸, z8, and R⁹ are as described herein, including in embodiments. At least one of W⁶ or W⁷ is N. If W⁶ is C(R⁶) or W⁷ is C(R⁷), then R¹⁰ is not hydrogen. If W⁶ and W⁷ are both N, then R³ is not —S(O)₂CH₃. If W⁶ is CH and W⁷ is N, then -L¹-R⁹ is not

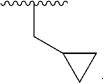 [SMILES: *CC1CC1]

R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁰ and R² substituents may optionally be joined to form a substituted or unsubstituted cycloalkyl, substituted or unsubstituted heterocycloalkyl, substituted or unsubstituted aryl, or substituted or unsubstituted heteroaryl.

R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl.

X¹⁰ is independently —F, —Cl, —Br, or —I.

The symbol n10 is an integer from 0 to 4. The symbols m10 and v10 are independently 1 or 2.

In an aspect is provided a pharmaceutical composition including a compound described herein, or a pharmaceutically acceptable salt or solvate thereof, and a pharmaceutically acceptable excipient.

In an aspect is provided a method of treating a neurodegenerative disorder in a subject in need thereof, the method including administering to the subject in need thereof a therapeutically effective amount of a compound described herein, or a pharmaceutically acceptable salt of solvate thereof.

In an aspect is provided a method of treating an inflammatory disease in a subject in need thereof, the method including administering to the subject in need thereof a therapeutically effective amount of a compound described herein, or a pharmaceutically acceptable salt of solvate thereof.

In an aspect is provided a method of treating a demyelinating disease in a subject in need thereof, the method including administering to the subject in need thereof a therapeutically effective amount of a compound described herein, or a pharmaceutically acceptable salt of solvate thereof.

In an aspect is provided a method of treating fibrotic disease in a subject in need thereof, the method including administering to the subject in need thereof a therapeutically effective amount of a compound described herein, or a pharmaceutically acceptable salt of solvate thereof.

In an aspect is provided a method of treating cancer in a subject in need thereof, the method including administering to the subject in need thereof a therapeutically effective amount of a compound described herein, or a pharmaceutically acceptable salt of solvate thereof.

In an aspect is provided a method of treating an LPAR1-associated disease in a subject in need thereof, the method including administering to the subject in need thereof a therapeutically effective amount of a compound described herein, or a pharmaceutically acceptable salt of solvate thereof.

In an aspect is provided a method of modulating LPAR1 activity in a subject, the method including administering to the subject a compound described herein, or a pharmaceutically acceptable salt or solvate thereof.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R’C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different.

Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

Bridged rings are two ore more rings that share three or more atoms, separating the two bridgehead atoms by a bridge containing at least one atom. Individual rings in bridged rings may be substituted or unsubstituted and may have different substituents from other individual rings within a set of bridged rings. Possible substituents for individual rings within bridged rings are the possible substituents for the same ring when not part of bridged rings (e.g., substituents for cycloalkyl or heterocycloalkyl rings). Bridged rings may be substituted or unsubstituted cycloalkyl, substituted or unsubstituted cycloalkylene, substituted or unsubstituted heterocycloalkyl or substituted or unsubstituted heterocycloalkylene and individual rings within a bridged ring group may be any of the immediately previous list, including having all rings of one type (e.g., all rings being substituted heterocycloalkylene wherein each ring may be the same or different substituted heterocycloalkylene). When referring to a bridged ring system, heterocyclic bridged rings means bridged rings wherein at least one ring is a heterocyclic ring and wherein each ring may be a different ring. When referring to a bridged ring system, substituted bridged rings means that at least one ring is substituted and each substituent may optionally be different.

The symbol “ [symbol] ” denotes the point of attachment of a chemical moiety to the [symbol] remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

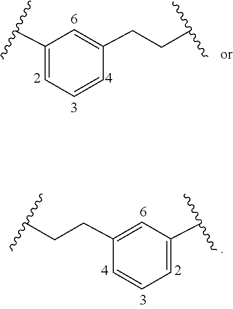 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, =N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)=NR″″, —NRC(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1} L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.1}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.1}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L4.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3}, . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

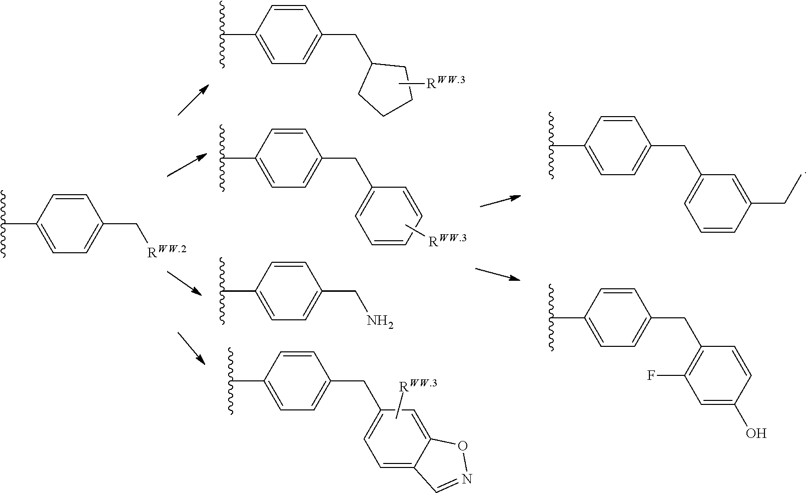

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂—CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl).

In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS), tert-butyldimethylsilyl (TBS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms.

In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing.

In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “lysophosphatidic acid receptor 1 antagonist” or “LPAR1 antagonist” refers to any exogenously administered compound or agent that is capable of partially or completely inhibiting, or reversing, the effect of an agonist (e.g., lysophosphatidic acid) on the LPAR1 receptor. The term is inclusive of compounds or agents characterized or described as antagonists, partial antagonists, and negative allosteric modulators.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a neurodegenerative disease. In embodiments, the disease is an inflammatory disease. In embodiments, the disease is post-hemorrhagic encephalitis. In embodiments, the disease is a demyelinating disease. In embodiments, the disease is multiple sclerosis. In embodiments, the disease is a fibrotic disease. In embodiments, the disease is pulmonary fibrosis. In embodiments, the disease is idiopathic pulmonary fibrosis. In embodiments, the disease is a cancer. In embodiments, the disease is glioblastoma.

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, scleroderma, and atopic dermatitis.

As used herein, the term “demyelinating disease” refers to any disease or condition characterized by damage to the protective covering (e.g., myelin sheath) that surrounds nerve fibers (e.g., in the brain, optic nerves, or spinal cord). In embodiments, the demyelinating disease is a demyelinating disease of the central nervous system. In embodiments, the demyelinating disease is multiple sclerosis. In embodiments, the demyelinating disease is a demyelinating disease of the peripheral nervous system.

As used herein, the terms “fibrotic disease” and “fibrosis” refer to any disease or condition characterized by the formation of excess fibrous connective tissue. The formation of excess fibrous connective tissue may be in response to a reparative or reactive process. Fibrotic diseases include but are not limited to pulmonary fibrosis (e.g., idiopathic pulmonary fibrosis (IPF)), liver fibrosis (e.g., nonalcoholic steatohepatitis (NASH)), myelofibrosis, skin fibrosis (e.g., scleroderma), ocular fibrosis, mediastinal fibrosis, cardiac fibrosis, kidney fibrosis, stromal fibrosis, epidural fibrosis, epithelial fibrosis, or idiopathic fibrosis.

As used herein, the term “cardiovascular disorder” or “cardiovascular disease” is used in accordance with its plain ordinary meaning. In embodiments, cardiovascular diseases that may be treated with a compound, pharmaceutical composition, or method described herein include, but are not limited to, stroke, heart failure, hypertension, hypertensive heart disease, myocardial infarction, angina pectoris, tachycardia, cardiomyopathy, rheumatic heart disease, cardiomyopathy, heart arrhythmia, congenital heart disease, valvular heart disease, carditis, aortic aneurysms, peripheral artery disease, thromboembolic disease, and venous thrombosis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “LPAR1-associated disease” refers to any disease or condition caused by aberrant activity or signaling of LPAR1. In embodiments, the LPAR1-associated disease is a neurodegenerative disease. In embodiments, the LPAR1-associated disease is an inflammatory disease. In embodiments, the LPAR1-associated disease is post-hemorrhagic encephalitis. In embodiments, the LPAR1-associated disease is a demyelinating disease. In embodiments, the LPAR1-associated disease is multiple sclerosis. In embodiments, the LPAR1-associated disease is a fibrotic disease. In embodiments, the LPAR1-associated disease is pulmonary fibrosis. In embodiments, the LPAR1-associated disease is idiopathic pulmonary fibrosis. In embodiments, the LPAR1-associated disease is a cancer. In embodiments, the LPAR1-associated disease is glioblastoma.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., multiple sclerosis, fibrotic disease, encephalitis, or cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., multiple sclerosis, fibrotic disease, encephalitis, or cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “lysophosphatidic acid receptor” or “LPAR” refers to one or more of the family of G protein-coupled receptors for lysophosphatidic acid (LPA). In embodiments, LPAR includes LPAR1, LPAR2, LPAR3, LPAR4, LPAR5, and LPAR6.

The term “lysophosphatidic acid receptor 1” or “LPAR1” refers to a G protein-coupled receptor (including homologs, isoforms, and functional fragments thereof) that binds the lipid signaling molecule lysophosphatidic acid (LPA). The term includes any recombinant or naturally-occurring form of LPAR1 variants thereof that maintain LPAR1 activity (e.g., within at least 30%, 40%, 50%, 60%, 70%, 80%, 90%, 95%, or 100% activity compared to wildtype LPAR1). In embodiments, the LPAR1 protein encoded by the LPAR1 gene has the amino acid sequence set forth in or corresponding to Entrez 1902, UniProt Q92633, RefSeq (protein) NP_001392.2 or RefSeq (protein) NP 476500.1. In embodiments, the LPAR1 gene has the nucleic acid sequence set forth in RefSeq (mRNA) NM_001401.3 or RefSeq (mRNA) NM_057159.2. In embodiments, the amino acid sequence or nucleic acid sequence is the sequence known at the time of filing of the present application.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward lysophosphatidic acid receptor 1 (LPAR1) would preferentially inhibit LPAR1 over other lysophosphatidic acid receptors). In embodiments, an “lysophosphatidic acid receptor 1 selective compound” or “LPAR1-selective compound” refers to a compound (e.g., compound described herein) having selectivity towards lysophosphatidic acid receptor 1 (LPAR1). In embodiments, the compound (e.g., compound described herein) is about 5-fold, 10-fold, 20-fold, 30-fold, 40-fold, 50-fold, or about 100-fold more selective for lysophosphatidic acid receptor 1 (LPAR1) over one or more of LPAR2, LPAR3, LPAR4, LPAR5, or LPAR6. In embodiments, the compound (e.g., compound described herein) is at least 5-fold, 10-fold, 20-fold, 30-fold, 40-fold, 50-fold, or at least 100-fold more selective for lysophosphatidic acid receptor 1 (LPAR1) over one or more of LPAR2, LPAR3, LPAR4, LPARS, or LPAR6.

### II. Compounds

In an aspect is provided a compound, or a pharmaceutically acceptable salt or solvate thereof, having the formula:

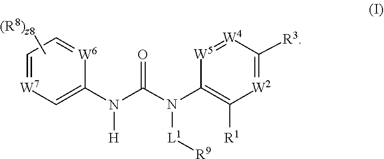

L¹ is a bond or substituted or unsubstituted alkylene (e.g., C₁-C₈, C₁-C₆, C₁-C₄, or C₁-C₂).

R¹ is substituted or unsubstituted alkyl (e.g., C₁-C₈, C₁-C₆, C₁-C₄, or C₁-C₂).

W² is N or C(R²).

R² is hydrogen, halogen, —CX²₃, —CHX²₂, —CH₂X², —OCX²₃, —OCH₂X², —OCHX²₂, —CN, —SOₙ₂R^{2D}, —SO₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —N^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

W⁴ is N or C(R⁴).

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

W⁵ is N or C(R⁵).

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² and R³ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ and R⁴ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁴ and R⁵ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

W⁶ is N or C(R⁶).

R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

W⁷ is N, N⁺—O⁻, or C(R⁷).

R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —N^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R⁸ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁹ is substituted or unsubstituted cycloalkyl (e.g., C₃-C₈, C₃-C₆, C₄-C₆, or C₅-C₆) or substituted or unsubstituted heterocycloalkyl (e.g., 3 to 8 membered, 3 to 6 membered, 4 to 6 membered, 4 to 5 membered, or 5 to 6 membered).

R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, and R^{8D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{5A} and R^{5B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{6A} and R^{6B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{7A} and R^{7B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{8A} and R^{8B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X², X³, X⁴, X⁵, X⁶, X⁷, and X⁸ are independently —F, —Cl, —Br, or —I.

The symbols n2, n3, n4, n5, n6, n7, and n8 are independently an integer from 0 to 4.

The symbols m2, m3, m4, m5, m6, m7, m8, v2, v3, v4, v5, v6, v7, and v8 are independently 1 or 2.

The symbol z8 is an integer from 0 to 3.

In embodiments, the compound has the formula:

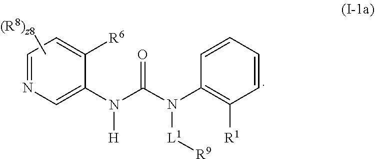

L¹, R¹, R⁶, R⁸, z8, and R⁹ are as described herein, including in embodiments.

In embodiments, the compound has the formula:

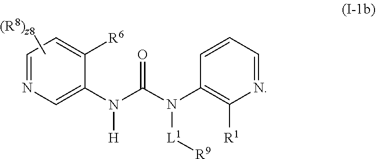

L¹, R¹, R⁶, R⁸, z8, and R⁹ are as described herein, including in embodiments.

In embodiments, the compound has the formula:

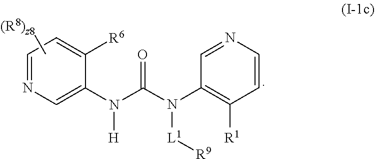

L¹, R¹, R⁶, R⁸, z8, and R⁹ are as described herein, including in embodiments.

In embodiments, the compound has the formula:

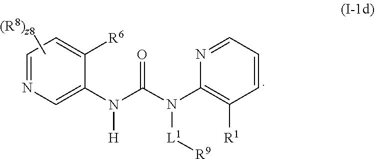

L¹, R¹, R⁶, R⁸, z8, and R⁹ are as described herein, including in embodiments.

In embodiments, the compound has the formula:

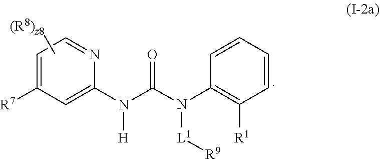

L¹, R¹, R⁷, R⁸, z8, and R⁹ are as described herein, including in embodiments.

In embodiments, the compound has the formula:

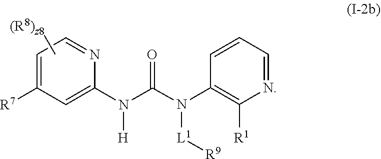

L¹, R¹, R⁷, R⁸, z8, and R⁹ are as described herein, including in embodiments.

In embodiments, the compound has the formula:

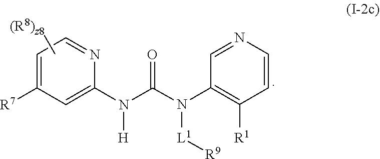

L¹, R¹, R⁷, R⁸, z8, and R⁹ are as described herein, including in embodiments.

In embodiments, the compound has the formula:

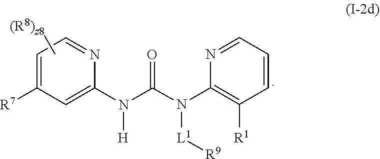

L¹, R¹, R⁶, R⁷, z8, and R⁹ are as described herein, including in embodiments.

In embodiments, the compound has the formula:

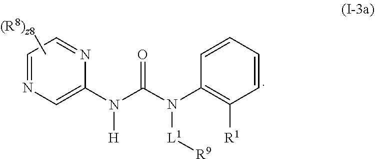

L¹, R¹, R⁸, z8, and R⁹ are as described herein, including in embodiments.

In embodiments, the compound has the formula:

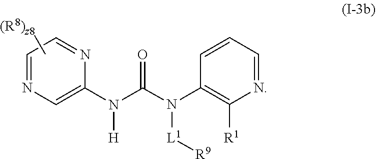

L¹, R¹, R⁸, z8, and R⁹ are as described herein, including in embodiments.

In embodiments, the compound has the formula:

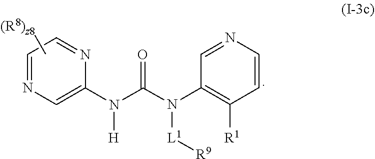

L¹, R¹, R⁸, z8, and R⁹ are as described herein, including in embodiments.

In embodiments, the compound has the formula:

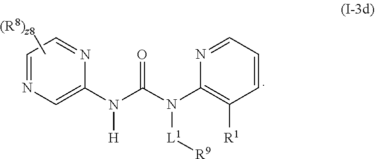

L¹, R¹, R⁸, z8, and R⁹ are as described herein, including in embodiments.

In embodiments, the compound has the formula:

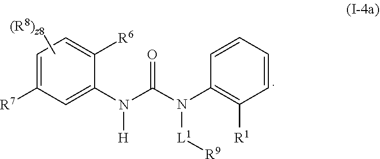

L¹, R¹, R⁶, R⁷, R⁸, z8, and R⁹ are as described herein, including in embodiments.

In embodiments, the compound has the formula:

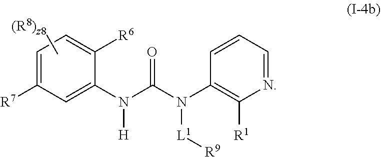

L¹, R¹, R⁶, R⁷, R⁸, z8, and R⁹ are as described herein, including in embodiments.

In embodiments, the compound has the formula:

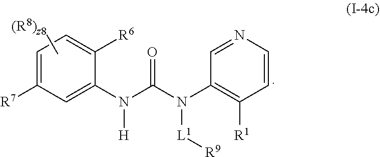

L¹, R¹, R⁶, R⁷, R⁸, z8, and R⁹ are as described herein, including in embodiments.

In embodiments, the compound has the formula:

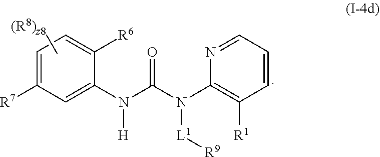

L¹, R¹, R⁶, R⁷, R⁸, z8, and R⁹ are as described herein, including in embodiments.

In embodiments, a substituted R¹ (e.g., substituted alkyl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is unsubstituted alkyl. In embodiments, R¹ is unsubstituted C₂-C₅ alkyl. In embodiments, R¹ is unsubstituted C₂ alkyl. In embodiments, R¹ is unsubstituted ethyl. In embodiments, R¹ is unsubstituted C₃ alkyl. In embodiments, R¹ is unsubstituted propyl. In embodiments, R¹ is unsubstituted n-propyl. In embodiments, R¹ is unsubstituted isopropyl (i.e., isopropyl). In embodiments, R¹ is unsubstituted C₄ alkyl. In embodiments, R¹ is unsubstituted butyl. In embodiments, R¹ is unsubstituted n-butyl. In embodiments, R¹ is unsubstituted isobutyl. In embodiments, R¹ is unsubstituted tert-butyl. In embodiments, R¹ is unsubstituted C₅ alkyl. In embodiments, R¹ is unsubstituted pentyl. In embodiments, R¹ is unsubstituted n-pentyl. In embodiments, R¹ is unsubstituted tert-pentyl. In embodiments, R¹ is unsubstituted neopentyl. In embodiments, R¹ is unsubstituted isopentyl.

In embodiments, R¹ is unsubstituted sec-pentyl. In embodiments, R¹ is unsubstituted 3-pentyl. In embodiments, R¹ is unsubstituted sec-isopentyl. In embodiments, R¹ is unsubstituted 2-methylbutyl.

In embodiments, W² is N. In embodiments, W² is C(R²). In embodiments, W² is CH.

In embodiments, W⁴ is N. In embodiments, W⁴ is C(R⁴). In embodiments, W⁴ is CH.

In embodiments, W⁵ is N. In embodiments, W⁵ is C(R⁵). In embodiments, W⁵ is CH.

In an aspect is provided a compound, or a pharmaceutically acceptable salt or solvate thereof, having the formula:

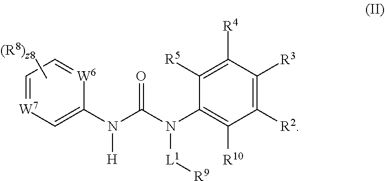

L¹, R², R³, R⁴, R⁵, W⁶, W⁷, R⁸, z8, and R⁹ are as described herein, including in embodiments. At least one of W⁶ or W⁷ is N. If W⁶ is C(R⁶) or W⁷ is C(R⁷), then R¹⁰ is not hydrogen. If W⁶ and W⁷ are both N, then R³ is not —S(O)₂CH₃. If W⁶ is CH and W⁷ is N, then -L¹-R⁹ is not

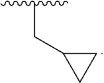 [SMILES: *CC1CC1]

R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁰ and R² substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{10A} and R^{10B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹⁰ is independently —F, —Cl, —Br, or —I.

The symbol n10 is an integer from 0 to 4.

The symbols m10 and v10 are independently 1 or 2.

In embodiments, the compound has the formula:

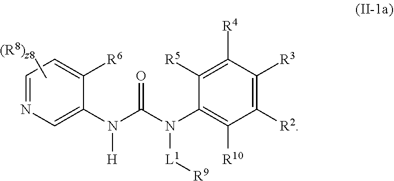

L¹, R², R³, R⁴, R⁵, R⁶, R⁸, z8, R⁹, and R¹⁰ are as described herein, including in embodiments.

In embodiments, the compound has the formula:

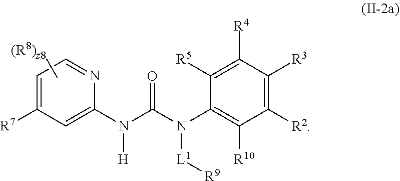

L¹, R², R³, R⁴, R⁵, R⁷, R⁸, z8, R⁹, and R¹⁰ are as described herein, including in embodiments.

In embodiments, the compound has the formula:

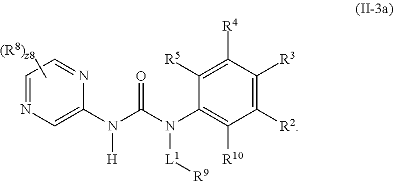

L¹, R², R³, R⁴, R⁵, R⁸, z8, R⁹, and R¹⁰ are as described herein, including in embodiments.

In embodiments, the compound has the formula:

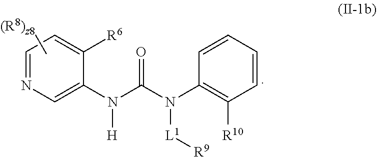

L¹, R⁶, R⁸, z8, R⁹, and R¹⁰ are as described herein, including in embodiments.

In embodiments, the compound has the formula:

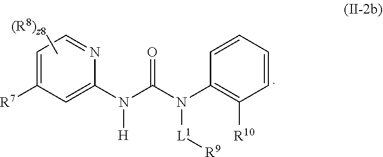

L¹, R⁷, R⁸, z8, R⁹, and R¹⁰ are as described herein, including in embodiments.

In embodiments, the compound has the formula:

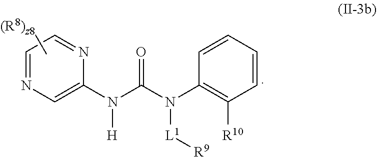

L¹, R⁸, z8, R⁹, and R¹⁰ are as described herein, including in embodiments.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, a substituted R^{10A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10A} is substituted with a plurality of groups selected from substituent groups, size-limited substituent groups, and lower substituent groups; each substituent group, size-limited substituent group, and/or lower substituent group may optionally be different. In embodiments, when R^{10A} is substituted, it is substituted with at least one substituent group. In embodiments, when R^{10A} is substituted, it is substituted with at least one size-limited substituent group. In embodiments, when R^{10A} is substituted, it is substituted with at least one lower substituent group.

In embodiments, a substituted R^{10B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10B} is substituted with a plurality of groups selected from substituent groups, size-limited substituent groups, and lower substituent groups; each substituent group, size-limited substituent group, and/or lower substituent group may optionally be different. In embodiments, when R^{10B} is substituted, it is substituted with at least one substituent group. In embodiments, when R^{10B} is substituted, it is substituted with at least one size-limited substituent group. In embodiments, when R^{10B} is substituted, it is substituted with at least one lower substituent group.

In embodiments, a substituted ring formed when R^{10A} and R^{10B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{10A} and R^{10B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{10A} and R^{10B} substituents bonded to the same nitrogen atom are joined is substituted, it is substituted with at least one substituent group. In embodiments, when the substituted ring formed when R^{10A} and R^{10B} substituents bonded to the same nitrogen atom are joined is substituted, it is substituted with at least one size-limited substituent group. In embodiments, when the substituted ring formed when R^{10A} and R^{10B} substituents bonded to the same nitrogen atom are joined is substituted, it is substituted with at least one lower substituent group.

In embodiments, a substituted R^{10C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10C} is substituted with a plurality of groups selected from substituent groups, size-limited substituent groups, and lower substituent groups; each substituent group, size-limited substituent group, and/or lower substituent group may optionally be different. In embodiments, when R^{10C} is substituted, it is substituted with at least one substituent group. In embodiments, when R^{10C} is substituted, it is substituted with at least one size-limited substituent group. In embodiments, when R^{10C} is substituted, it is substituted with at least one lower substituent group.

In embodiments, a substituted R^{10D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10D} is substituted with a plurality of groups selected from substituent groups, size-limited substituent groups, and lower substituent groups; each substituent group, size-limited substituent group, and/or lower substituent group may optionally be different. In embodiments, when R^{10D} is substituted, it is substituted with at least one substituent group. In embodiments, when R^{10D} is substituted, it is substituted with at least one size-limited substituent group. In embodiments, when R^{10D} is substituted, it is substituted with at least one lower substituent group.

In embodiments, R¹⁰ is hydrogen, halogen, —CCl₃, —CBr₃, —CF₃, —CI₃, —CHCl₂, —CHBr₂, —CHF₂, —CH₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁰ is hydrogen, —CHF₂, substituted or unsubstituted C₁-C₆ alkyl, substituted or unsubstituted 2 to 6 membered heteroalkyl, or substituted or unsubstituted C₃-C₈ cycloalkyl. In embodiments, R¹⁰ is hydrogen, —CHF₂, substituted or unsubstituted C₁-C₆ alkyl, or substituted or unsubstituted 2 to 6 membered heteroalkyl. In embodiments, R¹⁰ is hydrogen or unsubstituted C₁-C₆ alkyl. In embodiments, R¹⁰ is hydrogen. In embodiments, R¹⁰ is unsubstituted C₁ alkyl. In embodiments, R¹⁰ is unsubstituted methyl. In embodiments, R¹⁰ is unsubstituted C₂ alkyl. In embodiments, R¹⁰ is unsubstituted ethyl. In embodiments, R¹⁰ is unsubstituted C₃ alkyl. In embodiments, R¹⁰ is unsubstituted propyl. In embodiments, R¹⁰ is unsubstituted n-propyl. In embodiments, R¹⁰ is unsubstituted isopropyl (i.e., isopropyl). In embodiments, R¹⁰ is —CH(CD₃)₂. In embodiments, R¹⁰ is

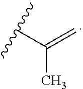 [SMILES: *C(=C)C]

In embodiments, R¹⁰ is unsubstituted C₄ alkyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl. In embodiments, R¹⁰ is unsubstituted C₅ alkyl.
In embodiments, R¹⁰ is unsubstituted pentyl. In embodiments, R¹⁰ is unsubstituted n-pentyl.
In embodiments, R¹⁰ is unsubstituted tert-pentyl. In embodiments, R¹⁰ is unsubstituted neopentyl. In embodiments, R¹⁰ is unsubstituted isopentyl. In embodiments, R¹⁰ is unsubstituted sec-pentyl. In embodiments, R¹⁰ is unsubstituted 3-pentyl. In embodiments, R¹⁰ is unsubstituted sec-isopentyl. In embodiments, R¹⁰ is unsubstituted 2-methylbutyl. In embodiments, R¹⁰ is unsubstituted C₆ alkyl. In embodiments, R¹⁰ is unsubstituted hexyl. In embodiments, R¹⁰ is substituted C₁-C₆ alkyl. In embodiments, R¹⁰ is substituted 2 to 6 membered heteroalkyl. In embodiments, R¹⁰ is —CH(CH₃)OCH₃. In embodiments, R¹⁰ is —CF(CH₃)₂. In embodiments, R¹⁰ is —C(CH₃)₂(OH). In embodiments, R¹⁰ is —CH(CH₃)(CH₂OH). In embodiments, R¹⁰ is —CHF₂. In embodiments, R¹⁰ is unsubstituted C₃-C₈ cycloalkyl. In embodiments, R¹⁰ is unsubstituted cyclopropyl. In embodiments, R¹⁰ is unsubstituted cyclobutyl. In embodiments, R¹⁰ is unsubstituted cyclopentyl. In embodiments, R¹⁰ is unsubstituted cyclohexyl. In embodiments, R¹⁰ is unsubstituted cycloheptyl. In embodiments, R¹⁰ is unsubstituted cyclooctyl.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen or unsubstituted C₁-C₆ alkyl. In embodiments, R² is hydrogen. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl. In embodiments, R² is unsubstituted pentyl. In embodiments, R² is unsubstituted hexyl.

In embodiments, a substituted ring formed when R¹⁰ and R² substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R¹⁰ and R² substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R¹⁰ and R² substituents are joined is substituted, it is substituted with at least one substituent group. In embodiments, when the substituted ring formed when R¹⁰ and R² substituents are joined is substituted, it is substituted with at least one size-limited substituent group. In embodiments, when the substituted ring formed when R¹⁰ and R² substituents are joined is substituted, it is substituted with at least one lower substituent group.

In embodiments, R¹⁰ and R² substituents may optionally be joined to form a substituted or unsubstituted heteroaryl. In embodiments, R¹⁰ and R² substituents may optionally be joined to form a substituted or unsubstituted 5 to 6 membered heteroaryl. In embodiments, R¹⁰ and R² substituents may optionally be joined to form a methyl-substituted 5 to 6 membered heteroaryl. In embodiments, R¹⁰ and R² substituents may optionally be joined to form

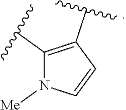 [SMILES: *c1ccn(C)c1*]

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A} and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen or unsubstituted C₁-C₆ alkyl. In embodiments, R³ is hydrogen. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl. In embodiments, R³ is unsubstituted pentyl. In embodiments, R³ is unsubstituted hexyl.

In embodiments, a substituted ring formed when R² and R³ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R² and R³ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R² and R³ substituents are joined is substituted, it is substituted with at least one substituent group. In embodiments, when the substituted ring formed when R² and R³ substituents are joined is substituted, it is substituted with at least one size-limited substituent group. In embodiments, when the substituted ring formed when R² and R³ substituents are joined is substituted, it is substituted with at least one lower substituent group.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen or unsubstituted C₁-C₆ alkyl. In embodiments, R⁴ is hydrogen. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is unsubstituted pentyl. In embodiments, R⁴ is unsubstituted hexyl.

In embodiments, a substituted ring formed when R³ and R⁴ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R³ and R⁴ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R³ and R⁴ substituents are joined is substituted, it is substituted with at least one substituent group. In embodiments, when the substituted ring formed when R³ and R⁴ substituents are joined is substituted, it is substituted with at least one size-limited substituent group. In embodiments, when the substituted ring formed when R³ and R⁴ substituents are joined is substituted, it is substituted with at least one lower substituent group.

In embodiments, a substituted R⁵(e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5A} and R^{5B} substituents bonded to the same nitrogen atom are joined is substituted, it is substituted with at least one substituent group. In embodiments, when the substituted ring formed when R^{5A} and R^{5B} substituents bonded to the same nitrogen atom are joined is substituted, it is substituted with at least one size-limited substituent group. In embodiments, when the substituted ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen or unsubstituted C₁-C₆ alkyl. In embodiments, R⁵ is hydrogen. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl. In embodiments, R⁵ is unsubstituted pentyl. In embodiments, R⁵ is unsubstituted hexyl.

In embodiments, a substituted ring formed when R⁴ and R⁵ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R⁴ and R⁵ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁴ and R⁵ substituents are joined is substituted, it is substituted with at least one substituent group. In embodiments, when the substituted ring formed when R⁴ and R⁵ substituents are joined is substituted, it is substituted with at least one size-limited substituent group. In embodiments, when the substituted ring formed when R⁴ and R⁵ substituents are joined is substituted, it is substituted with at least one lower substituent group.

In embodiments, W⁶ is N. In embodiments, W⁶ is C(R⁶). In embodiments, W⁶ is CH.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, a substituted R^{6A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, a substituted R^{6B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, a substituted ring formed when R^{6A} and R^{6B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{6A} and R^{6B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{6A} and R^{6B} substituents bonded to the same nitrogen atom are joined is substituted, it is substituted with at least one substituent group. In embodiments, when the substituted ring formed when R^{6A} and R^{6B} substituents bonded to the same nitrogen atom are joined is substituted, it is substituted with at least one size-limited substituent group. In embodiments, when the substituted ring formed when R^{6A} and R^{6B} substituents bonded to the same nitrogen atom are joined is substituted, it is substituted with at least one lower substituent group.

In embodiments, a substituted R^{6C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6C} is substituted with a plurality of groups selected from substituent groups, size-limited substituent groups, and lower substituent groups; each substituent group, size-limited substituent group, and/or lower substituent group may optionally be different. In embodiments, when R^{6C} is substituted, it is substituted with at least one substituent group. In embodiments, when R^{6C} is substituted, it is substituted with at least one size-limited substituent group. In embodiments, when R^{6C} is substituted, it is substituted with at least one lower substituent group.

In embodiments, a substituted R^{6D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6D} is substituted with a plurality of groups selected from substituent groups, size-limited substituent groups, and lower substituent groups; each substituent group, size-limited substituent group, and/or lower substituent group may optionally be different. In embodiments, when R^{6D} is substituted, it is substituted with at least one substituent group. In embodiments, when R^{6D} is substituted, it is substituted with at least one size-limited substituent group. In embodiments, when R^{6D} is substituted, it is substituted with at least one lower substituent group.

In embodiments, R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen, —OCHF₂, unsubstituted C₁-C₆ alkyl, or unsubstituted 2 to 6 membered heteroalkyl. In embodiments, R⁶ is hydrogen. In embodiments, R⁶ is —OCHF₂. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ is unsubstituted pentyl. In embodiments, R⁶ is unsubstituted hexyl. In embodiments, R⁶ is unsubstituted methoxy. In embodiments, R⁶ is unsubstituted ethoxy. In embodiments, R⁶ is unsubstituted proproxy. In embodiments, R⁶ is unsubstituted n-propoxy. In embodiments, R⁶ is unsubstituted isopropoxy. In embodiments, R⁶ is —OCD₃. In embodiments, R⁶ is unsubstituted butoxy. In embodiments, R⁶ is

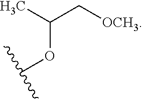 [SMILES: *OC(C)COC]

In embodiments, R⁶ is

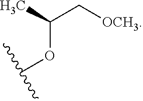 [SMILES: *O[C@@H](C)COC]

In embodiments, R⁶ is

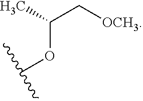 [SMILES: *O[C@H](C)COC]

In embodiments, W⁷ is N. In embodiments, W⁷ is N⁺—O⁻. In embodiments, W⁷ is C(R⁷). In embodiments, W⁷ is CH.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, a substituted R^{7A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, a substituted R^{7B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different. In embodiments, when R^{7B} is substituted, it is substituted with at least one substituent group. In embodiments, when R^{7B} is substituted, it is substituted with at least one size-limited substituent group. In embodiments, when R^{7B} is substituted, it is substituted with at least one lower substituent group.

In embodiments, a substituted ring formed when R^{7A} and R^{7B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{7A} and R^{7B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{7A} and R^{7B} substituents bonded to the same nitrogen atom are joined is substituted, it is substituted with at least one substituent group. In embodiments, when the substituted ring formed when R^{7A} and R^{7B} substituents bonded to the same nitrogen atom are joined is substituted, it is substituted with at least one size-limited substituent group. In embodiments, when the substituted ring formed when R^{7A} and R^{7B} substituents bonded to the same nitrogen atom are joined is substituted, it is substituted with at least one lower substituent group.

In embodiments, a substituted R^{7C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7C} is substituted with a plurality of groups selected from substituent groups, size-limited substituent groups, and lower substituent groups; each substituent group, size-limited substituent group, and/or lower substituent group may optionally be different. In embodiments, when R^{7C} is substituted, it is substituted with at least one substituent group. In embodiments, when R^{7C} is substituted, it is substituted with at least one size-limited substituent group. In embodiments, when R^{7C} is substituted, it is substituted with at least one lower substituent group.

In embodiments, a substituted R^{7D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7D} is substituted with a plurality of groups selected from substituent groups, size-limited substituent groups, and lower substituent groups; each substituent group, size-limited substituent group, and/or lower substituent group may optionally be different. In embodiments, when R^{7D} is substituted, it is substituted with at least one substituent group. In embodiments, when R^{7D} is substituted, it is substituted with at least one size-limited substituent group. In embodiments, when R^{7D} is substituted, it is substituted with at least one lower substituent group.

In embodiments, R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is hydrogen, halogen, —OCHF₂, or unsubstituted C₁-C₆ alkyl. In embodiments, R⁷ is hydrogen. In embodiments, R⁷ is halogen. In embodiments, R⁷ is —F. In embodiments, R⁷ is —Cl. In embodiments, R⁷ is —Br. In embodiments, R⁷ is —OCF₂. In embodiments, R⁷ is unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl. In embodiments, R⁷ is unsubstituted pentyl. In embodiments, R⁷ is unsubstituted hexyl.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, a substituted R^{8A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8A} is substituted with a plurality of groups selected from substituent groups, size-limited substituent groups, and lower substituent groups; each substituent group, size-limited substituent group, and/or lower substituent group may optionally be different. In embodiments, when R^{8A} is substituted, it is substituted with at least one substituent group. In embodiments, when R^{8A} is substituted, it is substituted with at least one size-limited substituent group. In embodiments, when R^{8A} is substituted, it is substituted with at least one lower substituent group.

In embodiments, a substituted R^{8B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8B} is substituted with a plurality of groups selected from substituent groups, size-limited substituent groups, and lower substituent groups; each substituent group, size-limited substituent group, and/or lower substituent group may optionally be different. In embodiments, when R^{8B} is substituted, it is substituted with at least one substituent group. In embodiments, when R^{8B} is substituted, it is substituted with at least one size-limited substituent group. In embodiments, when R^{8B} is substituted, it is substituted with at least one lower substituent group.

In embodiments, a substituted ring formed when R^{8A} and R^{8B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{8A} and R^{8B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{8A} and R^{8B} substituents bonded to the same nitrogen atom are joined is substituted, it is substituted with at least one substituent group. In embodiments, when the substituted ring formed when R^{8A} and R^{8B} substituents bonded to the same nitrogen atom are joined is substituted, it is substituted with at least one size-limited substituent group. In embodiments, when the substituted ring formed when R^{8A} and R^{8B} substituents bonded to the same nitrogen atom are joined is substituted, it is substituted with at least one lower substituent group.

In embodiments, a substituted R^{8C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8C} is substituted with a plurality of groups selected from substituent groups, size-limited substituent groups, and lower substituent groups; each substituent group, size-limited substituent group, and/or lower substituent group may optionally be different. In embodiments, when R^{8C} is substituted, it is substituted with at least one substituent group. In embodiments, when R^{8C} is substituted, it is substituted with at least one size-limited substituent group. In embodiments, when R^{8C} is substituted, it is substituted with at least one lower substituent group.

In embodiments, a substituted R^{8D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8D} is substituted with a plurality of groups selected from substituent groups, size-limited substituent groups, and lower substituent groups; each substituent group, size-limited substituent group, and/or lower substituent group may optionally be different. In embodiments, when R^{8D} is substituted, it is substituted with at least one substituent group. In embodiments, when R^{8D} is substituted, it is substituted with at least one size-limited substituent group. In embodiments, when R^{8D} is substituted, it is substituted with at least one lower substituent group.

In embodiments, R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl.

In embodiments, R⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁸ is independently halogen, —CF₃, —CHF₂, —CN, —OCHF₂, —C(O)R^{8C}, —C(O)OR^{8C}, —OR^{8D}, unsubstituted C₁-C₆ alkyl, unsubstituted 2 to 8 membered heteroalkyl, unsubstituted C₃-C₈ cycloalkyl, or unsubstituted phenyl.

In embodiments, R^{8C} is independently hydrogen or unsubstituted C₁-C₆ alkyl. In embodiments, R^{8C} is independently hydrogen. In embodiments, R^{8C} is independently unsubstituted methyl. In embodiments, R^{8C} is independently unsubstituted ethyl. In embodiments, R^{8C} is independently unsubstituted propyl. In embodiments, R^{8C} is independently unsubstituted n-propyl. In embodiments, R^{8C} is independently unsubstituted isopropyl. In embodiments, R^{8C} is independently unsubstituted butyl. In embodiments, R^{8C} is independently unsubstituted n-butyl. In embodiments, R^{8C} is independently unsubstituted isobutyl. In embodiments, R^{8C} is independently unsubstituted tert-butyl. In embodiments, R^{8C} is independently unsubstituted pentyl. In embodiments, R^{8C} is independently unsubstituted hexyl.

In embodiments, R^{8D} is independently hydrogen or unsubstituted C₁-C₆ alkyl. In embodiments, R^{8D} is independently hydrogen. In embodiments, R^{8D} is independently unsubstituted methyl. In embodiments, R^{8D} is independently unsubstituted ethyl. In embodiments, R^{8D} is independently unsubstituted propyl. In embodiments, R^{8D} is independently unsubstituted n-propyl. In embodiments, R^{8D} is independently unsubstituted isopropyl. In embodiments, R^{8D} is independently unsubstituted butyl. In embodiments, R^{8D} is independently unsubstituted n-butyl. In embodiments, R^{8D} is independently unsubstituted isobutyl. In embodiments, R^{8D} is independently unsubstituted tert-butyl. In embodiments, R^{8D} is independently unsubstituted pentyl. In embodiments, R^{8D} is independently unsubstituted hexyl.

In embodiments, R⁸ is independently —F, —Cl, —Br, —CF₃, —CHF₂, —CN, —C(O)H, —OCHF₂, —OCH₃, —OCH₂CH₃, —OCH₂CF₃, —OCH(CH₃)CH₂OCH₃, —OCH₂CHF₂, unsubstituted methyl, unsubstituted cyclopropyl, or unsubstituted phenyl.

In embodiments, R⁸ is independently —F, —Cl, —Br, —CF₃, —CHF₂, —OH, —CN, —C(O)H, —OCHF₂, —OCH₃, —OCH₂CH₃, —OCH₂CF₃, —OCH(CH₃)CH₂OCH₃, —OCH₂CHF₂, substituted or unsubstituted methyl, unsubstituted cyclopropyl, or unsubstituted phenyl.

In embodiments, R⁸ is independently —F. In embodiments, R⁸ is independently —Cl.

In embodiments, R⁸ is independently —Br. In embodiments, R⁸ is independently —CF₃. In embodiments, R⁸ is independently —CHF₂. In embodiments, R⁸ is independently —OH. In embodiments, R⁸ is independently —CN. In embodiments, R⁸ is independently —C(O)H. In embodiments, R⁸ is independently —C(O)OCH₃. In embodiments, R⁸ is independently —OCHF₂. In embodiments, R⁸ is independently unsubstituted C₁-C₆ alkyl. In embodiments, R⁸ is independently unsubstituted methyl. In embodiments, R⁸ is independently unsubstituted ethyl. In embodiments, R⁸ is independently unsubstituted propyl. In embodiments, R⁸ is independently unsubstituted n-propyl. In embodiments, R⁸ is independently unsubstituted isopropyl. In embodiments, R⁸ is independently unsubstituted butyl. In embodiments, R⁸ is independently unsubstituted n-butyl. In embodiments, R⁸ is independently unsubstituted isobutyl. In embodiments, R⁸ is independently unsubstituted tert-butyl. In embodiments, R⁸ is independently unsubstituted pentyl. In embodiments, R⁸ is independently unsubstituted hexyl. In embodiments, R⁸ is independently substituted C₁-C₆ alkyl. In embodiments, R⁸ is independently substituted methyl. In embodiments, R⁸ is independently substituted ethyl. In embodiments, R⁸ is independently substituted propyl. In embodiments, R⁸ is independently substituted n-propyl. In embodiments, R⁸ is independently substituted isopropyl. In embodiments, R⁸ is independently substituted butyl. In embodiments, R⁸ is independently substituted n-butyl. In embodiments, R⁸ is independently substituted isobutyl. In embodiments, R⁸ is independently substituted tert-butyl. In embodiments, R⁸ is independently substituted pentyl. In embodiments, R⁸ is independently substituted hexyl. In embodiments, R⁸ is independently —CH₂-(unsubstituted phenyl). In embodiments, R⁸ is independently substituted or unsubstituted 2 to 8 membered heteroalkyl.
In embodiments, R⁸ is independently unsubstituted methoxy. In embodiments, R⁸ is independently —OCD₃. In embodiments, R⁸ is independently unsubstituted ethoxy. In embodiments, R⁸ is independently unsubstituted propoxy. In embodiments, R⁸ is independently unsubstituted n-propoxy. In embodiments, R⁸ is independently unsubstituted isopropoxy. In embodiments, R⁸ is independently unsubstituted butoxy. In embodiments, R⁸ is independently —CH₂OCH₃. In embodiments, R⁸ is independently —OCH₂CF₃. In embodiments, R⁸ is independently —OCH₂CHF₂. In embodiments, R⁸ is independently —OCH₂CH₂F. In embodiments, R⁸ is independently

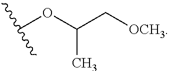 [SMILES: *OC(C)COC]

In embodiments, R⁸ is independently

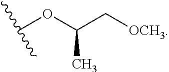 [SMILES: *O[C@H](C)COC]

In embodiments, R⁸ is independently

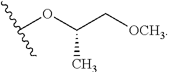 [SMILES: *O[C@@H](C)COC]

In embodiments, R⁸ is independently unsubstituted C₃-C₈ cycloalkyl. In embodiments, R⁸ is independently unsubstituted cyclopropyl. In embodiments, R⁸ is independently unsubstituted cyclobutyl. In embodiments, R⁸ is independently unsubstituted cyclopentyl. In embodiments, R⁸ is independently unsubstituted cyclohexyl. In embodiments, R⁸ is independently unsubstituted phenyl.

In embodiments, two R⁸ substituents are joined to form an unsubstituted C₃-C₈ cycloalkyl. In embodiments, two R⁸ substituents are joined to form an unsubstituted C₃ cycloalkyl. In embodiments, two R⁸ substituents are joined to form an unsubstituted C₄ cycloalkyl. In embodiments, two R⁸ substituents are joined to form an unsubstituted C₅ cycloalkyl. In embodiments, two R⁸ substituents are joined to form an unsubstituted C₆ cycloalkyl. In embodiments, two R⁸ substituents are joined to form an unsubstituted C₇ cycloalkyl. In embodiments, two R⁸ substituents are joined to form an unsubstituted C₈ cycloalkyl.

In embodiments, z8 is 0. In embodiments, z8 is 1. In embodiments, z8 is 2. In embodiments, z8 is 3.

In embodiments,

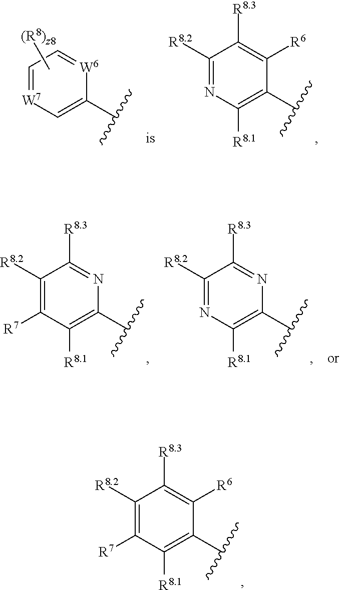

wherein R⁶ and R⁷ are as described herein, including in embodiments. R^{8.1}, R^{8.2}, and R^{8.3} are independently hydrogen or any value of R⁸ as described herein, including in embodiments.

In embodiments,

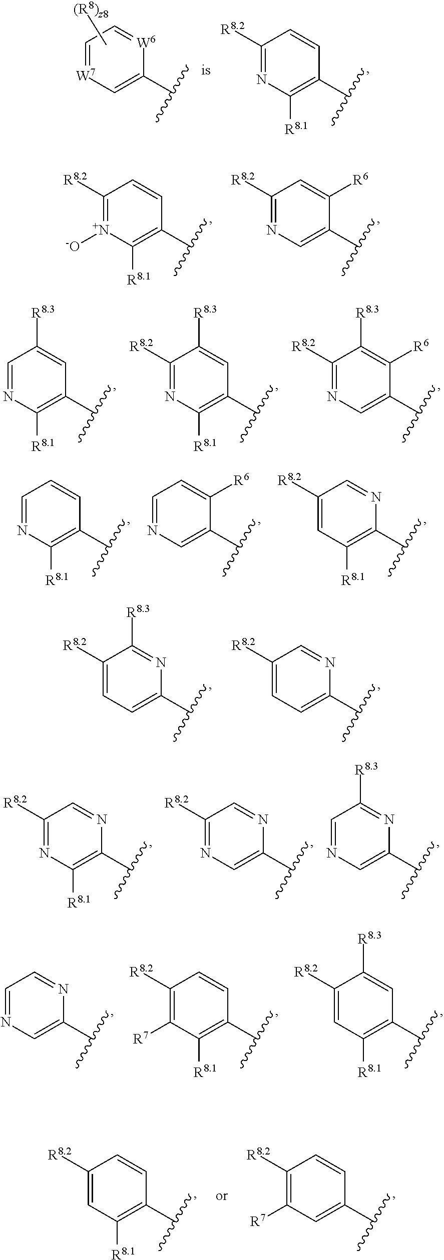

wherein R⁶ and R⁷ are as described herein, including in embodiments. R^{8.1}, R^{8.2}, and R^{8.3} are independently hydrogen or any value of R⁸ as described herein, including in embodiments.

In embodiments,

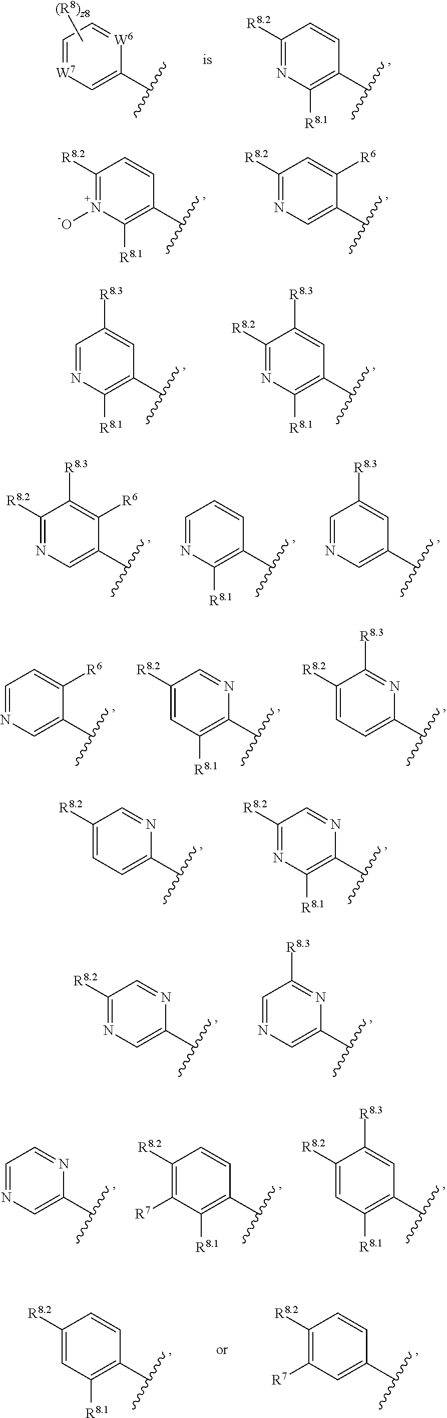

wherein R⁶ and R⁷ are as described herein, including in embodiments. R^{8.1}, R^{8.2}, and R^{8.3} are independently hydrogen or any value of R⁸ as described herein, including in embodiments.

In embodiments, a substituted R^{8.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8.1} is substituted with a plurality of groups selected from substituent groups, size-limited substituent groups, and lower substituent groups; each substituent group, size-limited substituent group, and/or lower substituent group may optionally be different. In embodiments, when R^{8.1} is substituted, it is substituted with at least one substituent group. In embodiments, when R^{8.1} is substituted, it is substituted with at least one size-limited substituent group. In embodiments, when R^{8.1} is substituted, it is substituted with at least one lower substituent group.

In embodiments, a substituted R^{8.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8.2} is substituted with a plurality of groups selected from substituent groups, size-limited substituent groups, and lower substituent groups; each substituent group, size-limited substituent group, and/or lower substituent group may optionally be different. In embodiments, when R^{8.2} is substituted, it is substituted with at least one substituent group. In embodiments, when R^{8.2} is substituted, it is substituted with at least one size-limited substituent group. In embodiments, when R^{8.2} is substituted, it is substituted with at least one lower substituent group.

In embodiments, a substituted R^{8.3} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8.3} is substituted with a plurality of groups selected from substituent groups, size-limited substituent groups, and lower substituent groups; each substituent group, size-limited substituent group, and/or lower substituent group may optionally be different. In embodiments, when R^{8.3} is substituted, it is substituted with at least one substituent group. In embodiments, when R^{8.3} is substituted, it is substituted with at least one size-limited substituent group. In embodiments, when R^{8.3} is substituted, it is substituted with at least one lower substituent group.

In embodiments, a substituted ring formed when R^{8.2} and R^{8.3} substituents are joined (e.g., substituted cycloalkyl and/or substituted heterocycloalkyl) is substituted with at least one substituent group, size-limited substituent group, or lower substituent group; wherein if the substituted ring formed when R^{8.2} and R^{8.3} substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{8.2} and R^{8.3} substituents are joined is substituted, it is substituted with at least one substituent group. In embodiments, when the substituted ring formed when R^{8.2} and R^{8.3} substituents are joined is substituted, it is substituted with at least one size-limited substituent group. In embodiments, when the substituted ring formed when R^{8.2} and R^{8.3} substituents are joined is substituted, it is substituted with at least one lower substituent group.

In embodiments, R^{8.1}, R^{8.2}, and R^{8.3} are independently hydrogen, halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —N^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{8.2} and R^{8.3} substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{8.1}, R^{8.2}, and R^{8.3} are independently hydrogen, halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{8.2} and R^{8.3} substituents may optionally be joined to form a substituted or unsubstituted cycloalkyl, substituted or unsubstituted heterocycloalkyl, substituted or unsubstituted aryl, or substituted or unsubstituted heteroaryl.

In embodiments, R^{8.1}, R^{8.2}, and R^{8.3} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{8.1}, R^{8.2}, and R^{8.3} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{8.1} is —F. In embodiments, R^{8.1} is —Cl. In embodiments, R^{8.1} is —Br. In embodiments, R^{8.1} is —CF₃. In embodiments, R^{8.1} is —CHF₂. In embodiments, R^{8.1} is —CN. In embodiments, R^{8.1} is —C(O)H. In embodiments, R^{8.1} is —C(O)OCH₃. In embodiments, R^{8.1} is —OCHF₂. In embodiments, R^{8.1} is unsubstituted C₁-C₆ alkyl. In embodiments, R^{8.1} is unsubstituted methyl. In embodiments, R^{8.1} is unsubstituted ethyl. In embodiments, R^{8.1} is unsubstituted propyl. In embodiments, R^{8.1} is unsubstituted n-propyl.

In embodiments, R^{8.1} is unsubstituted isopropyl. In embodiments, R^{8.1} is unsubstituted butyl.
In embodiments, R^{8.1} is unsubstituted n-butyl. In embodiments, R^{8.1} is unsubstituted isobutyl.
In embodiments, R^{8.1} is unsubstituted tert-butyl. In embodiments, R^{8.1} is unsubstituted pentyl. In embodiments, R^{8.1} is unsubstituted hexyl. In embodiments, R^{8.1} is substituted C₁-C₆ alkyl. In embodiments, R^{8.1} is substituted methyl. In embodiments, R^{8.1} is substituted ethyl. In embodiments, R^{8.1} is substituted propyl. In embodiments, R^{8.1} is substituted n-propyl. In embodiments, R^{8.1} is substituted isopropyl. In embodiments, R^{8.1} is substituted butyl. In embodiments, R^{8.1} is substituted n-butyl. In embodiments, R^{8.1} is substituted isobutyl. In embodiments, R^{8.1} is substituted tert-butyl. In embodiments, R^{8.1} is substituted pentyl. In embodiments, R^{8.1} is substituted hexyl. In embodiments, R^{8.1} is —CH₂-(unsubstituted phenyl). In embodiments, R^{8.1} is substituted or unsubstituted 2 to 8 membered heteroalkyl. In embodiments, R^{8.1} is unsubstituted methoxy. In embodiments, R^{8.1} is —OCD₃. In embodiments, R^{8.1} is unsubstituted ethoxy. In embodiments, R^{8.1} is unsubstituted propoxy. In embodiments, R^{8.1} is unsubstituted n-propoxy. In embodiments, R^{8.1} is unsubstituted isopropoxy. In embodiments, R^{8.1} is unsubstituted butoxy. In embodiments, R^{8.1} is —CH₂OCH₃. In embodiments, R^{8.1} is —OCH₂CF₃. In embodiments, R^{8.1} is —OCH₂CHF₂. In embodiments, R^{8.1} is —OCH₂CH₂F. In embodiments, R^{8.1} is

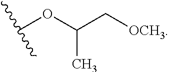 [SMILES: *OC(C)COC]

In embodiments, R^{8.1} is

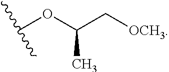 [SMILES: *O[C@H](C)COC]

In embodiments, R^{8.1} is

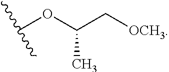 [SMILES: *O[C@@H](C)COC]

In embodiments, R^{8.1} is unsubstituted C₃-C₈ cycloalkyl. In embodiments, R^{8.1} is unsubstituted cyclopropyl. In embodiments, R^{8.1} is unsubstituted cyclobutyl. In embodiments, R^{8.1} is unsubstituted cyclopentyl. In embodiments, R^{8.1} is unsubstituted cyclohexyl. In embodiments, R^{8.1} is unsubstituted phenyl.

In embodiments, R^{8.2} is —F. In embodiments, R^{8.2} is —Cl. In embodiments, R^{8.2} is —Br. In embodiments, R^{8.2} is —CF₃. In embodiments, R^{8.2} is —CHF₂. In embodiments, R^{8.2} is —CN. In embodiments, R^{8.2} is —C(O)H. In embodiments, R^{8.2} is —C(O)OCH₃. In embodiments, R^{8.2} is —OCHF₂. In embodiments, R^{8.2} is unsubstituted C₁-C₆ alkyl. In embodiments, R^{8.2} is unsubstituted methyl. In embodiments, R^{8.2} is unsubstituted ethyl. In embodiments, R^{8.2} is unsubstituted propyl. In embodiments, R^{8.2} is unsubstituted n-propyl.

In embodiments, R^{8.2} is unsubstituted isopropyl. In embodiments, R^{8.2} is unsubstituted butyl.
In embodiments, R^{8.2} is unsubstituted n-butyl. In embodiments, R^{8.2} is unsubstituted isobutyl.
In embodiments, R^{8.2} is unsubstituted tert-butyl. In embodiments, R^{8.2} is unsubstituted pentyl. In embodiments, R^{8.2} is unsubstituted hexyl. In embodiments, R^{8.2} is substituted C₁-C₆ alkyl. In embodiments, R^{8.2} is substituted methyl. In embodiments, R^{8.2} is substituted ethyl. In embodiments, R^{8.2} is substituted propyl. In embodiments, R^{8.2} is substituted n-propyl. In embodiments, R^{8.2} is substituted isopropyl. In embodiments, R^{8.2} is substituted butyl. In embodiments, R^{8.2} is substituted n-butyl. In embodiments, R^{8.2} is substituted isobutyl. In embodiments, R^{8.2} is substituted tert-butyl. In embodiments, R^{8.2} is substituted pentyl. In embodiments, R^{8.2} is substituted hexyl. In embodiments, R^{8.2} is —CH₂-(unsubstituted phenyl). In embodiments, R^{8.2} is substituted or unsubstituted 2 to 8 membered heteroalkyl. In embodiments, R^{8.2} is unsubstituted methoxy. In embodiments, R^{8.2} is —OCD₃. In embodiments, R^{8.2} is unsubstituted ethoxy. In embodiments, R^{8.2} is unsubstituted propoxy. In embodiments, R^{8.2} is unsubstituted n-propoxy. In embodiments, R^{8.2} is unsubstituted isopropoxy. In embodiments, R^{8.2} is unsubstituted butoxy. In embodiments, R^{8.2} is —CH₂OCH₃. In embodiments, R^{8.2} is —OCH₂CF₃. In embodiments, R^{8.2} is —OCH₂CHF₂. In embodiments, R^{8.2} is —OCH₂CH₂F. In embodiments, R^{8.2} is

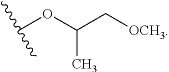 [SMILES: *OC(C)COC]

In embodiments, R^{8.2} is

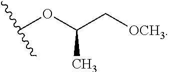 [SMILES: *O[C@H](C)COC]

In embodiments, R^{8.2} is

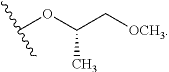 [SMILES: *O[C@@H](C)COC]

In embodiments, R^{8.2} is unsubstituted C₃-C₈ cycloalkyl. In embodiments, R^{8.2} is unsubstituted cyclopropyl. In embodiments, R^{8.2} is unsubstituted cyclobutyl. In embodiments, R^{8.2} is unsubstituted cyclopentyl. In embodiments, R^{8.2} is unsubstituted cyclohexyl. In embodiments, R^{8.2} is unsubstituted phenyl.

In embodiments, R^{8.3} is —F. In embodiments, R^{8.3} is —Cl. In embodiments, R^{8.3} is —Br. In embodiments, R^{8.3} is —CF₃. In embodiments, R^{8.3} is —CHF₂. In embodiments, R^{8.3} is —CN. In embodiments, R^{8.3} is —C(O)H. In embodiments, R^{8.3} is —C(O)OCH₃. In embodiments, R^{8.3} is —OCHF₂. In embodiments, R^{8.3} is unsubstituted C₁-C₆ alkyl. In embodiments, R^{8.3} is unsubstituted methyl. In embodiments, R^{8.3} is unsubstituted ethyl. In embodiments, R^{8.3} is unsubstituted propyl. In embodiments, R^{8.3} is unsubstituted n-propyl.

In embodiments, R^{8.3} is unsubstituted isopropyl. In embodiments, R^{8.3} is unsubstituted butyl.
In embodiments, R^{8.3} is unsubstituted n-butyl. In embodiments, R^{8.3} is unsubstituted isobutyl.
In embodiments, R^{8.3} is unsubstituted tert-butyl. In embodiments, R^{8.3} is unsubstituted pentyl. In embodiments, R^{8.3} is unsubstituted hexyl. In embodiments, R^{8.3} is substituted C₁-C₆ alkyl. In embodiments, R^{8.3} is substituted methyl. In embodiments, R^{8.3} is substituted ethyl. In embodiments, R^{8.3} is substituted propyl. In embodiments, R^{8.3} is substituted n-propyl. In embodiments, R^{8.3} is substituted isopropyl. In embodiments, R^{8.3} is substituted butyl. In embodiments, R^{8.3} is substituted n-butyl. In embodiments, R^{8.3} is substituted isobutyl. In embodiments, R^{8.3} is substituted tert-butyl. In embodiments, R^{8.3} is substituted pentyl. In embodiments, R^{8.3} is substituted hexyl. In embodiments, R^{8.3} is —CH₂-(unsubstituted phenyl). In embodiments, R^{8.3} is substituted or unsubstituted 2 to 8 membered heteroalkyl. In embodiments, R^{8.3} is unsubstituted methoxy. In embodiments, R^{8.3} is —OCD₃. In embodiments, R^{8.3} is unsubstituted ethoxy. In embodiments, R^{8.3} is unsubstituted propoxy. In embodiments, R^{8.3} is unsubstituted n-propoxy. In embodiments, R^{8.3} is unsubstituted isopropoxy. In embodiments, R^{8.3} is unsubstituted butoxy. In embodiments, R^{8.3} is —CH₂OCH₃. In embodiments, R^{8.3} is —OCH₂CF₃. In embodiments, R^{8.3} is —OCH₂CHF₂. In embodiments, R^{8.3} is —OCH₂CH₂F. In embodiments, R^{8.3} is

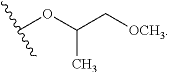 [SMILES: *OC(C)COC]

In embodiments, R^{8.3} is

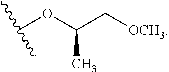 [SMILES: *O[C@H](C)COC]

In embodiments, R^{8.3} is

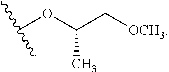 [SMILES: *O[C@@H](C)COC]

In embodiments, R^{8.3} is unsubstituted C₃-C₈ cycloalkyl. In embodiments, R^{8.3} is unsubstituted cyclopropyl. In embodiments, R^{8.3} is unsubstituted cyclobutyl. In embodiments, R^{8.3} is unsubstituted cyclopentyl. In embodiments, R^{8.3} is unsubstituted cyclohexyl. In embodiments, R^{8.3} is unsubstituted phenyl.

In embodiments, R^{8.2} and R^{8.3} substituents are joined to form an unsubstituted C₃-C₈ cycloalkyl. In embodiments, R^{8.2} and R^{8.3} substituents are joined to form an unsubstituted C₃ cycloalkyl. In embodiments, R^{8.2} and R^{8.3} substituents are joined to form an unsubstituted C₄ cycloalkyl. In embodiments, R^{8.2} and R^{8.3} substituents are joined to form an unsubstituted C₅ cycloalkyl. In embodiments, R^{8.2} and R^{8.3} substituents are joined to form an unsubstituted C₆ cycloalkyl. In embodiments, R^{8.2} and R^{8.3} substituents are joined to form an unsubstituted C₇ cycloalkyl. In embodiments, R^{8.2} and R^{8.3} substituents are joined to form an unsubstituted C₈ cycloalkyl.

In embodiments,

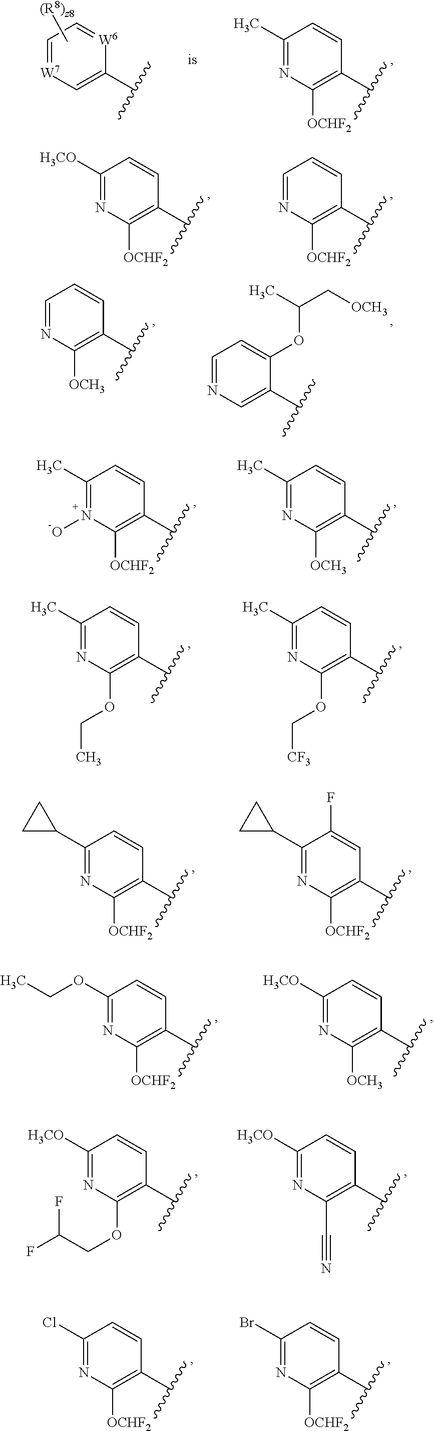

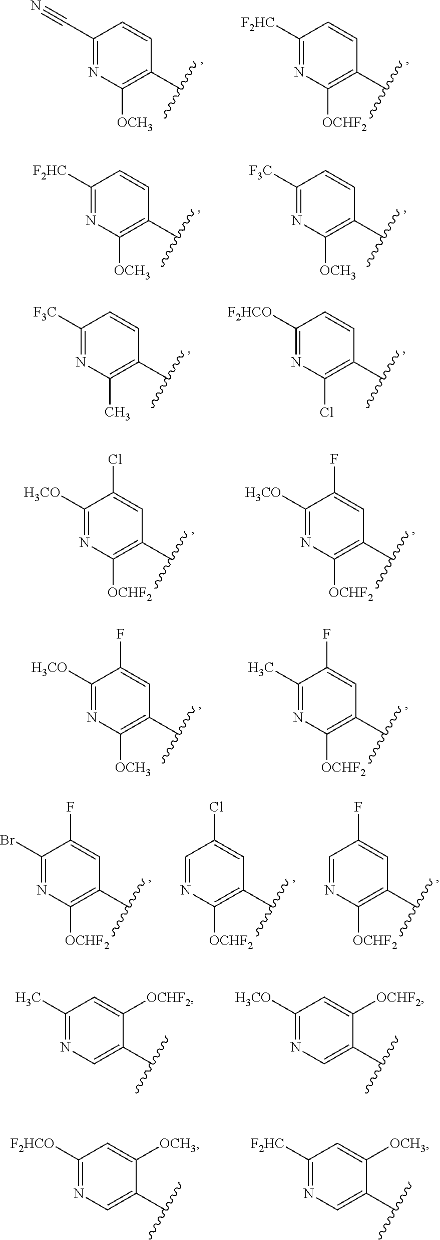

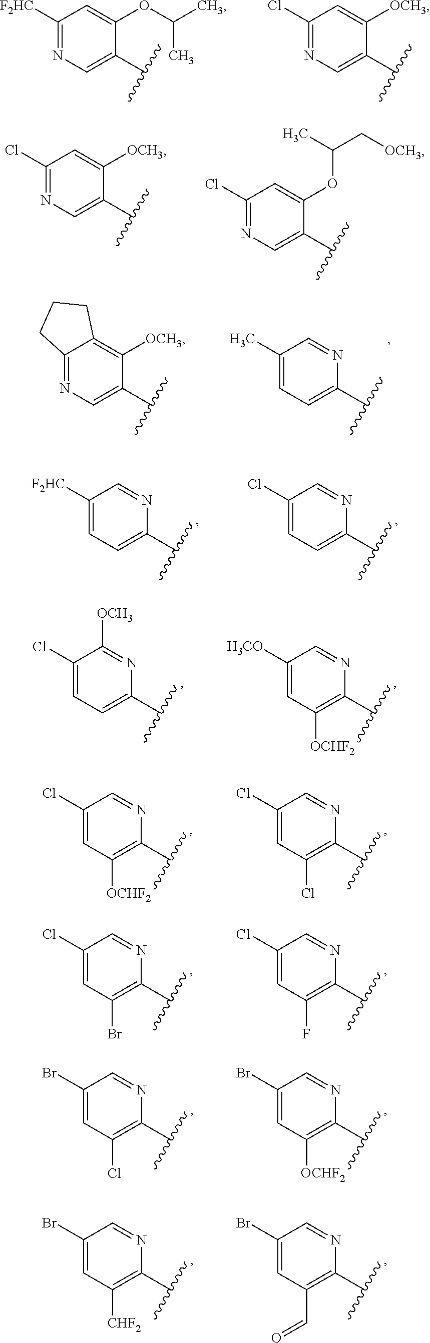

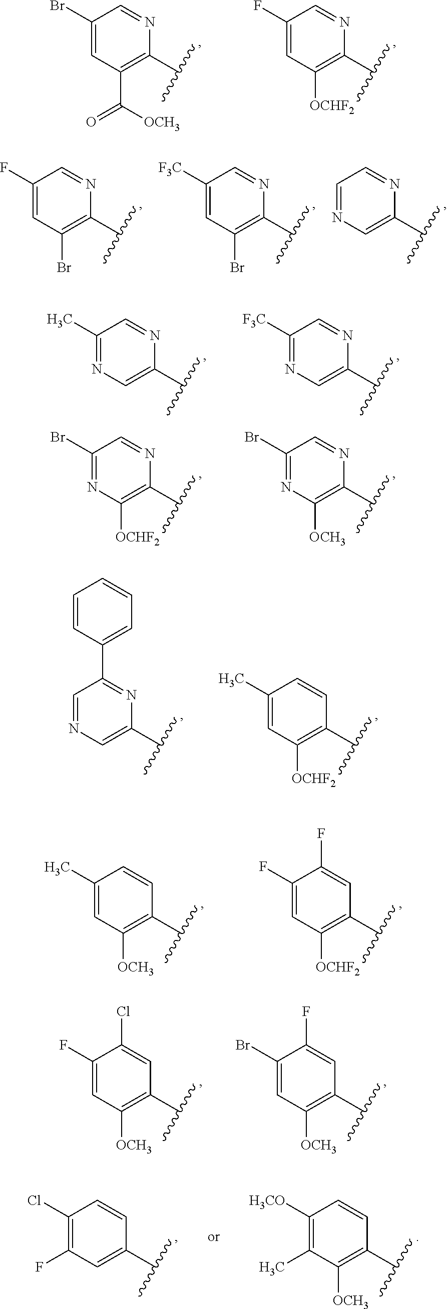

In embodiments,

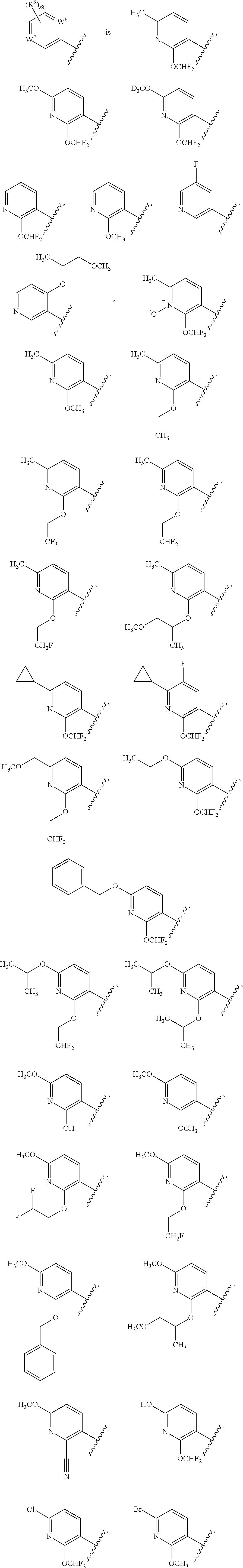

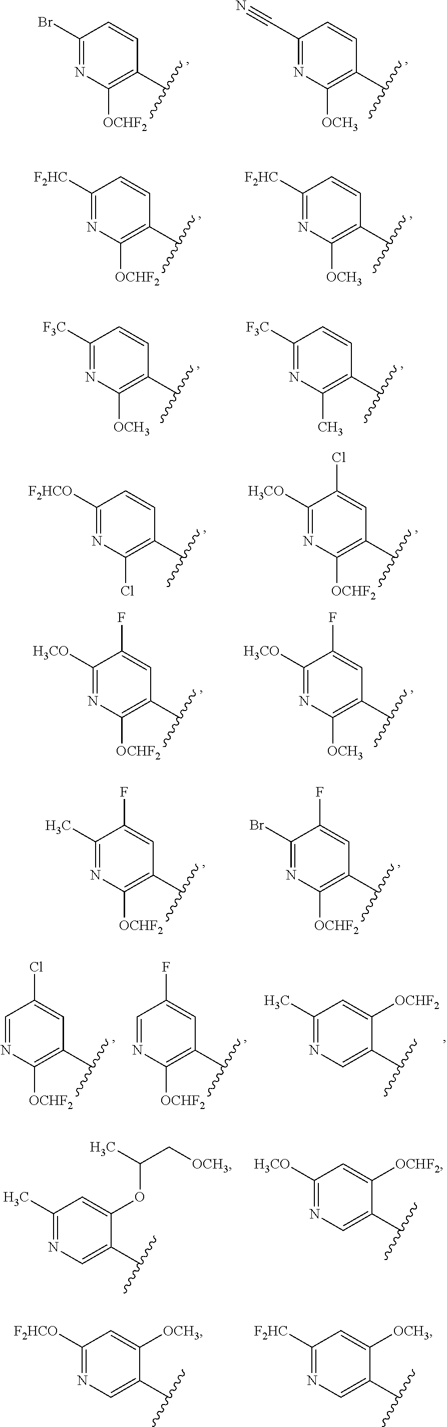

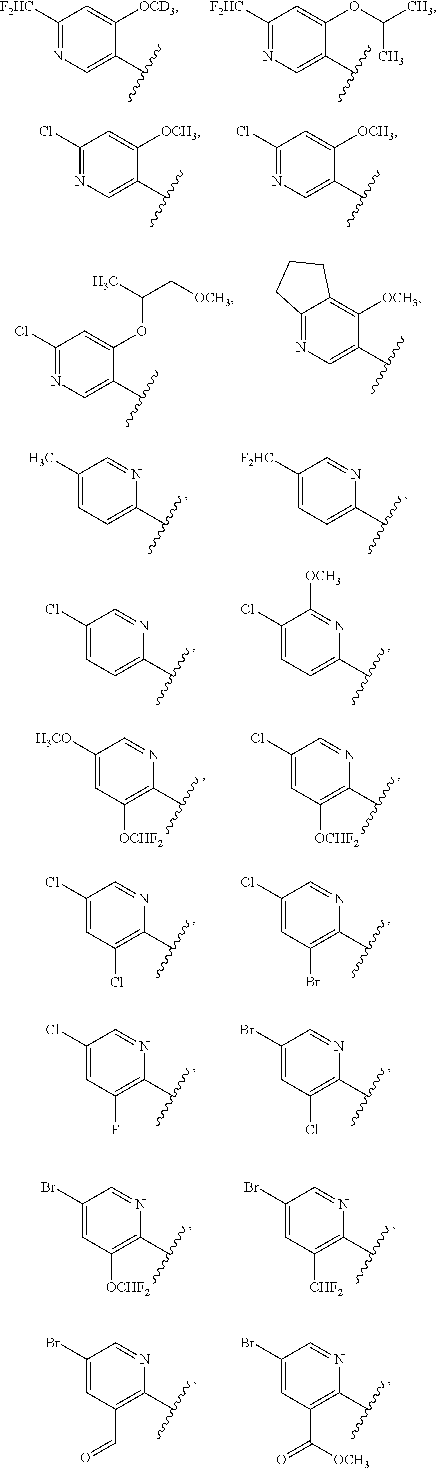

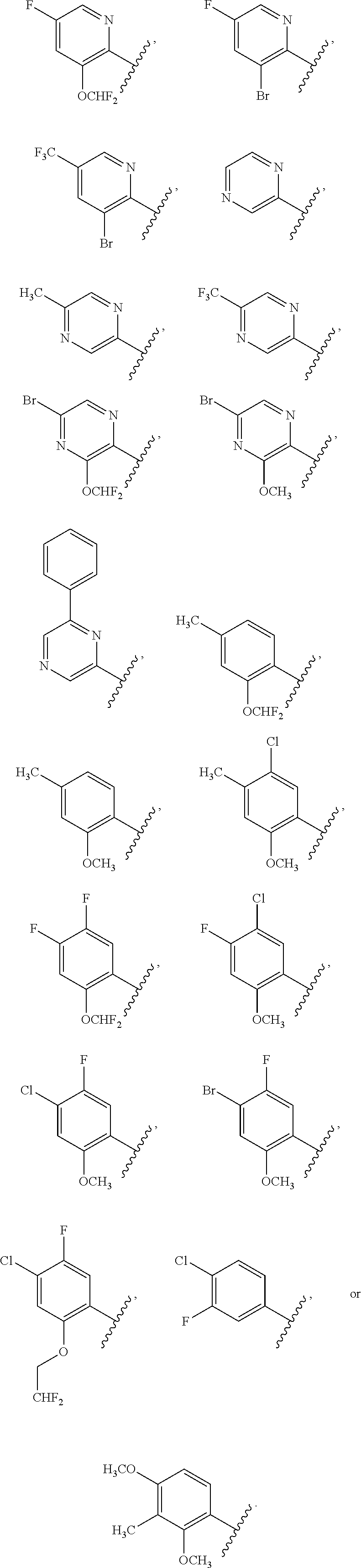

In embodiments,

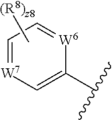

is

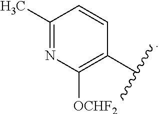 [SMILES: *c1ccc(C)nc1OC(F)F]

In embodiments,

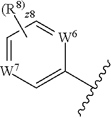

is

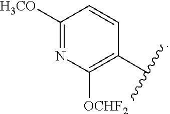 [SMILES: *c1ccc(OC)nc1OC(F)F]

In embodiments,

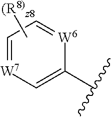

is

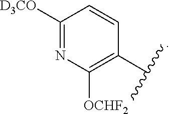

In embodiments,

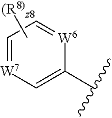

is

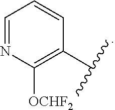 [SMILES: *c1cccnc1OC(F)F]

In embodiments,

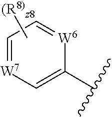

is

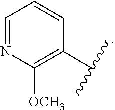 [SMILES: *c1cccnc1OC]

In embodiments,

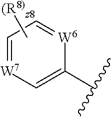

is

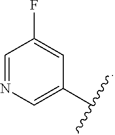 [SMILES: *c1cncc(F)c1]

In embodiments,

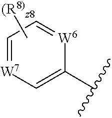

is

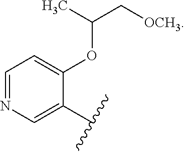 [SMILES: *c1cnccc1OC(C)COC]

In embodiments,

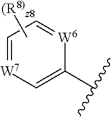

is

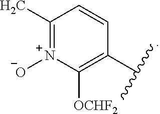 [SMILES: *c1ccc(C)[n+]([O-])c1OC(F)F]

In embodiments,

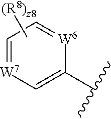

is

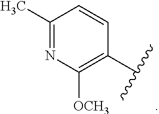 [SMILES: *c1ccc(C)nc1OC]

In embodiments,

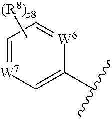

is

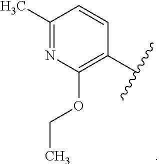 [SMILES: *c1ccc(C)nc1OCC]

In embodiments,

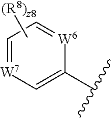

is

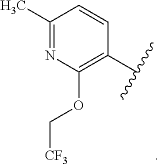 [SMILES: *c1ccc(C)nc1OCC(F)(F)F]

In embodiments,

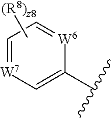

is

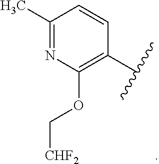 [SMILES: *c1ccc(C)nc1OCC(F)F]

In embodiments,

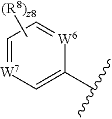

is

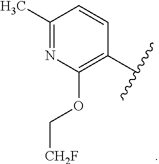

In embodiments,

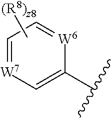

is

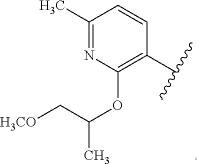 [SMILES: *c1ccc(C)nc1OC(C)COC]

In embodiments,

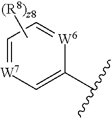

is

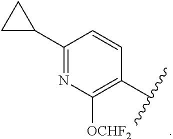 [SMILES: *c1ccc(C2CC2)nc1OC(F)F]

In embodiments,

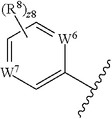

is

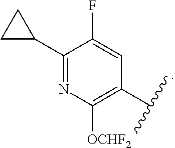 [SMILES: *c1cc(F)c(C2CC2)nc1OC(F)F]

In embodiments,

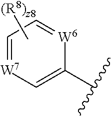

is

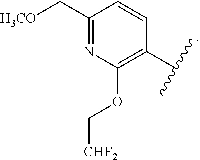 [SMILES: *c1ccc(COC)nc1OCC(F)F]

In embodiments,

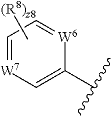

is

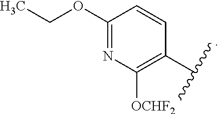 [SMILES: *c1ccc(OCC)nc1OC(F)F]

In embodiments,

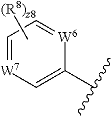

is

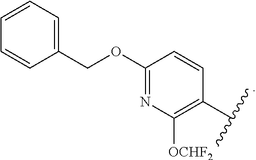 [SMILES: *c1ccc(OCc2ccccc2)nc1OC(F)F]

In embodiments,

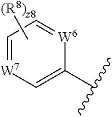

is

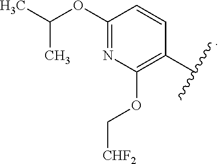 [SMILES: *c1ccc(OC(C)C)nc1OCC(F)F]

In embodiments,

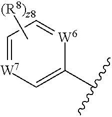

is

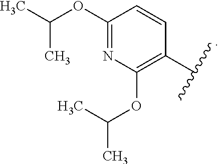 [SMILES: *c1ccc(OC(C)C)nc1OC(C)C]

In embodiments,

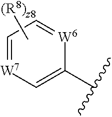

is

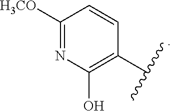 [SMILES: *c1ccc(OC)nc1O]

In embodiments,

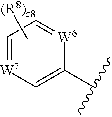

is

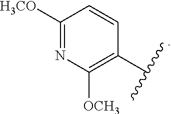 [SMILES: *c1ccc(OC)nc1OC]

In embodiments,

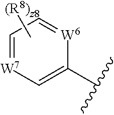

is

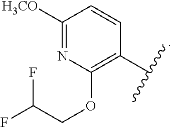 [SMILES: *c1ccc(OC)nc1OCC(F)F]

In embodiments,

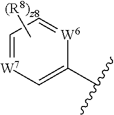

is

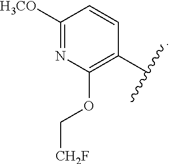

In embodiments,

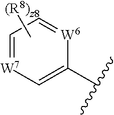

is

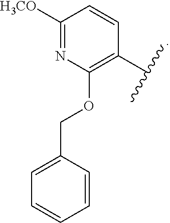 [SMILES: *c1ccc(OC)nc1OCc1ccccc1]

In embodiments,

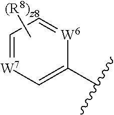

is

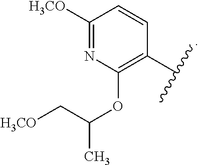 [SMILES: *c1ccc(OC)nc1OC(C)COC]

In embodiments,

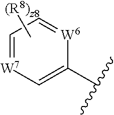

is

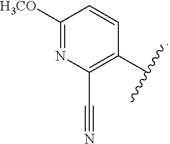 [SMILES: *c1ccc(OC)nc1C#N]

In embodiments,

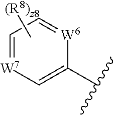

is

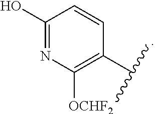 [SMILES: *c1ccc(O)nc1OC(F)F]

In embodiments,

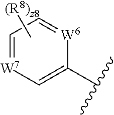

is

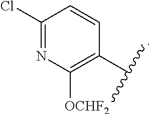 [SMILES: *c1ccc(Cl)nc1OC(F)F]

In embodiments,

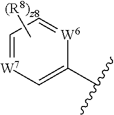

is

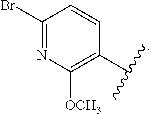 [SMILES: *c1ccc(Br)nc1OC]

In embodiments,

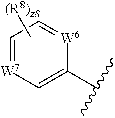

is

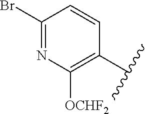 [SMILES: *c1ccc(Br)nc1OC(F)F]

In embodiments,

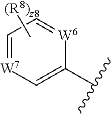

is

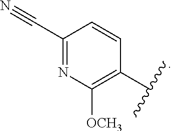 [SMILES: *c1ccc(C#N)nc1OC]

In embodiments,

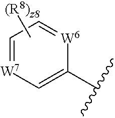

is

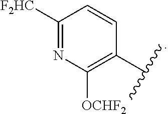 [SMILES: *c1ccc(C(F)F)nc1OC(F)F]

In embodiments,

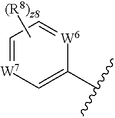

is

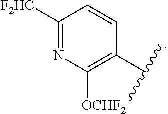 [SMILES: *c1ccc(C(F)F)nc1OC(F)F]

In embodiments,

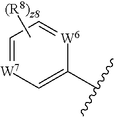

is

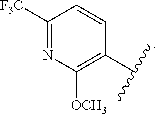 [SMILES: *c1ccc(C(F)(F)F)nc1OC]

In embodiments,

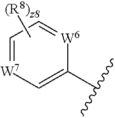

is

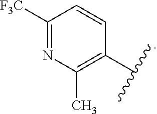 [SMILES: *c1ccc(C(F)(F)F)nc1C]

In embodiments,

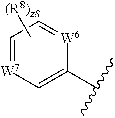

is

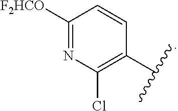 [SMILES: *c1ccc(OC(F)F)nc1Cl]

In embodiments,

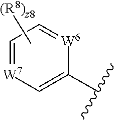

is

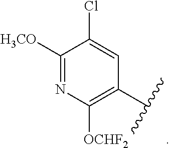 [SMILES: *c1cc(Cl)c(OC)nc1OC(F)F]

In embodiments,

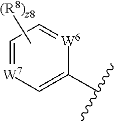

is

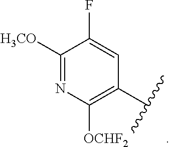 [SMILES: *c1cc(F)c(OC)nc1OC(F)F]

In embodiments,

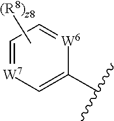

is

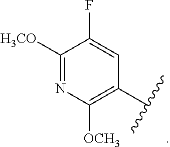 [SMILES: *c1cc(F)c(OC)nc1OC]

In embodiments,

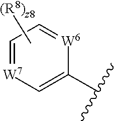

is

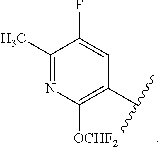 [SMILES: *c1cc(F)c(C)nc1OC(F)F]

In embodiments,

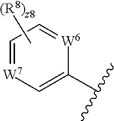

is

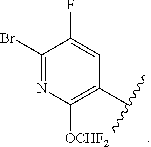 [SMILES: *c1cc(F)c(Br)nc1OC(F)F]

In embodiments,

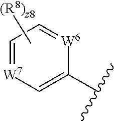

is

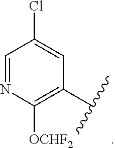 [SMILES: *c1cc(Cl)cnc1OC(F)F]

In embodiments,

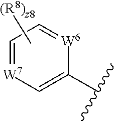

is

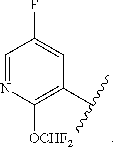 [SMILES: *c1cc(F)cnc1OC(F)F]

In embodiments,

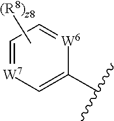

is

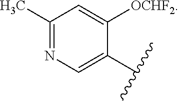 [SMILES: *c1cnc(C)cc1OC(F)F]

In embodiments,

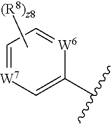

is

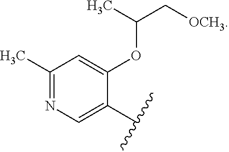 [SMILES: *c1cnc(C)cc1OC(C)COC]

In embodiments,

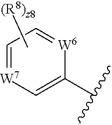

is

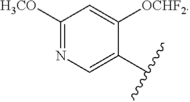 [SMILES: *c1cnc(OC)cc1OC(F)F]

In embodiments,

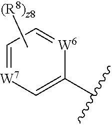

is

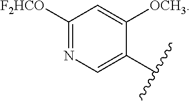 [SMILES: *c1cnc(OC(F)F)cc1OC]

In embodiments,

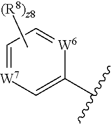

is

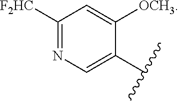 [SMILES: *c1cnc(C(F)F)cc1OC]

In embodiments,

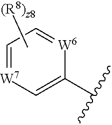

is

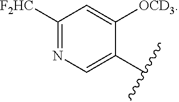

In embodiments,

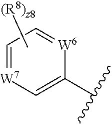

is

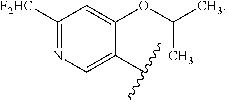 [SMILES: *c1cnc(C(F)F)cc1OC(C)C]

In embodiments,

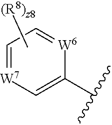

is

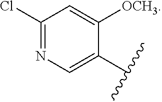 [SMILES: *c1cnc(Cl)cc1OC]

In embodiments,

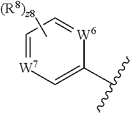

is

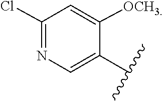 [SMILES: *c1cnc(Cl)cc1OC]

In embodiments,

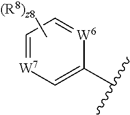

is

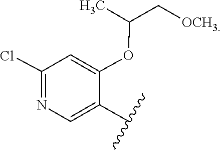 [SMILES: *c1cnc(Cl)cc1OC(C)COC]

In embodiments,

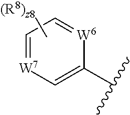

is

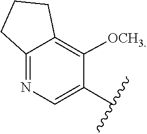 [SMILES: *c1cnc2c(c1OC)CCC2]

In embodiments,

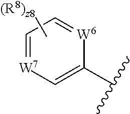

is

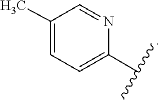 [SMILES: *c1ccc(C)cn1]

In embodiments,

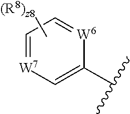

is

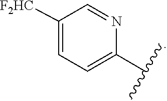 [SMILES: *c1ccc(C(F)F)cn1]

In embodiments,

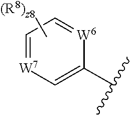

is

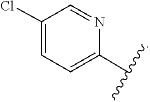 [SMILES: *c1ccc(Cl)cn1]

In embodiments,

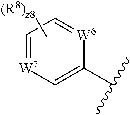

is

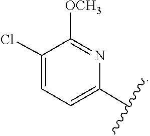 [SMILES: *c1ccc(Cl)c(OC)n1]

In embodiments,

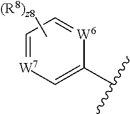

is

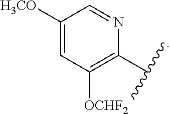 [SMILES: *c1ncc(OC)cc1OC(F)F]

In embodiments,

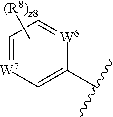

is

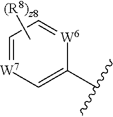

In embodiments,

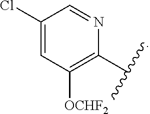 [SMILES: *c1ncc(Cl)cc1OC(F)F]

is

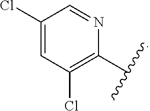 [SMILES: *c1ncc(Cl)cc1Cl]

In embodiments,

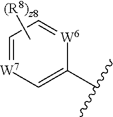

is

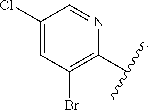 [SMILES: *c1ncc(Cl)cc1Br]

In embodiments,

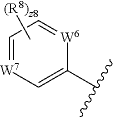

is

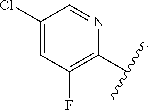 [SMILES: *c1ncc(Cl)cc1F]

In embodiments,

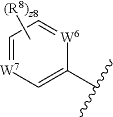

is

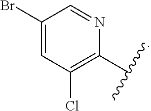 [SMILES: *c1ncc(Br)cc1Cl]

In embodiments,

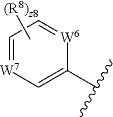

is

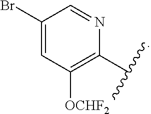 [SMILES: *c1ncc(Br)cc1OC(F)F]

In embodiments,

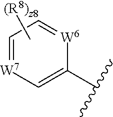

is

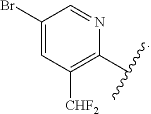 [SMILES: *c1ncc(Br)cc1C(F)F]

In embodiments,

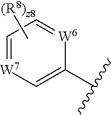

is

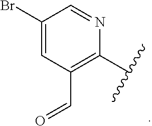 [SMILES: *c1ncc(Br)cc1C=O]

In embodiments,

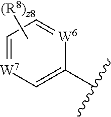

is

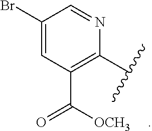 [SMILES: *c1ncc(Br)cc1C(=O)OC]

In embodiments,

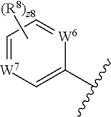

is

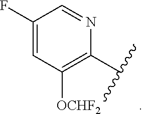 [SMILES: *c1ncc(F)cc1OC(F)F]

In embodiments,

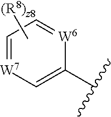

is

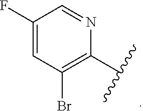 [SMILES: *c1ncc(F)cc1Br]

In embodiments,

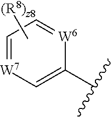

is

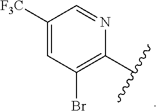 [SMILES: *c1ncc(C(F)(F)F)cc1Br]

In embodiments,

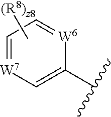

is

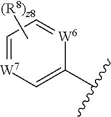

In embodiments,

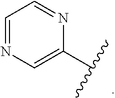 [SMILES: *c1cnccn1]

is

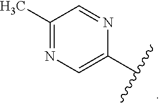 [SMILES: *c1cnc(C)cn1]

In embodiments,

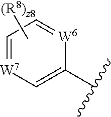

is

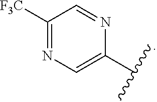 [SMILES: *c1cnc(C(F)(F)F)cn1]

In embodiments,

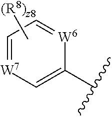

is

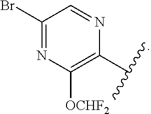 [SMILES: *c1ncc(Br)nc1OC(F)F]

In embodiments,

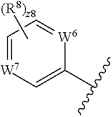

is

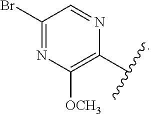 [SMILES: *c1ncc(Br)nc1OC]

In embodiments,

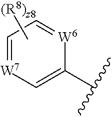

is

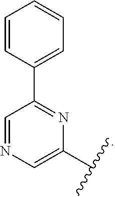 [SMILES: *c1cncc(-c2ccccc2)n1]

In embodiments,

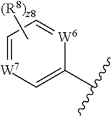

is

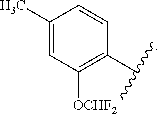 [SMILES: *c1ccc(C)cc1OC(F)F]

In embodiments,

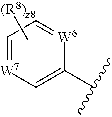

is

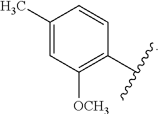 [SMILES: *c1ccc(C)cc1OC]

In embodiments,

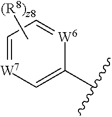

is

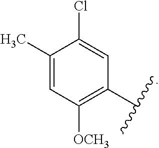 [SMILES: *c1cc(Cl)c(C)cc1OC]

In embodiments,

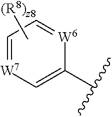

is

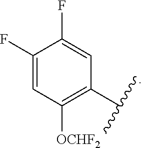 [SMILES: *c1cc(F)c(F)cc1OC(F)F]

In embodiments,

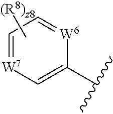

is

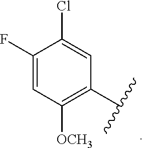 [SMILES: *c1cc(Cl)c(F)cc1OC]

In embodiments,

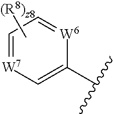

is

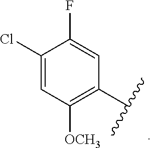 [SMILES: *c1cc(F)c(Cl)cc1OC]

In embodiments,

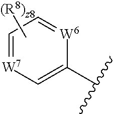

is

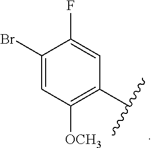 [SMILES: *c1cc(F)c(Br)cc1OC]

In embodiments,

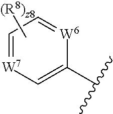

is

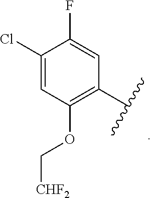 [SMILES: *c1cc(F)c(Cl)cc1OCC(F)F]

In embodiments,

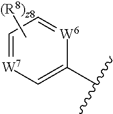

is

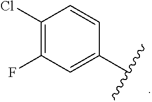 [SMILES: *c1ccc(Cl)c(F)c1]

In embodiments,

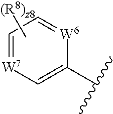

is

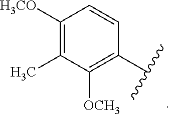 [SMILES: *c1ccc(OC)c(C)c1OC]

In embodiments, a substituted L¹ (e.g., substituted alkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond or substituted or unsubstituted C₁-C₅ alkylene. In embodiments, L¹ is a bond or unsubstituted C₁-C₅ alkylene. In embodiments, L¹ is a bond.

In embodiments, L¹ is unsubstituted methylene. In embodiments, L¹ is unsubstituted ethylene. In embodiments, L¹ is unsubstituted propylene. In embodiments, L¹ is unsubstituted butylene. In embodiments, L¹ is unsubstituted pentylene.

In embodiments, a substituted R⁹ (e.g., substituted cycloalkylene and/or substituted heterocycloalkylene) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is substituted or unsubstituted C₃-C₈ cycloalkyl or substituted or unsubstituted 3 to 8 membered heterocycloalkyl.

In embodiments, R⁹ is an R¹¹-substituted or unsubstituted cycloalkyl or R¹¹-substituted or unsubstituted heterocycloalkyl.

R¹¹ is independently oxo, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR^{11C}NR^{11A}R^{11B}, —ONR^{11A}R^{11B}, —NHC(O)NR^{11C}NR^{11A}R^{11B}, —NHC(O)NR^{11A}R^{11B}, —N(O)ₘ₁₁, —NR^{11A}R^{11B}, —C(O)R^{11C}, —C(O)OR^{11C}, —C(O)NR^{11A}R^{11B}, —OR^{11D}, —SR^{11D}, —NR^{11A}SO₂R^{11D}, —NR^{11A}C(O)R^{11C}, —NR^{11A}C(O)OR^{11C}, —NR^{11A}OR^{11C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{11A}, R^{11B}, R^{11C}, and R^{11D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{11A} and R^{11B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹¹ is independently —F, —Cl, —Br, or —I.

The symbol n11 is independently an integer from 0 to 4.

The symbols m11 and v11 are independently 1 or 2.

In embodiments, R⁹ is an R¹¹-substituted or unsubstituted C₃-C₈ cycloalkyl or R¹¹-substituted or unsubstituted 3 to 8 membered heterocycloalkyl. In embodiments, R⁹ is an R¹¹-substituted or unsubstituted spirocyclic cycloalkyl or R¹¹-substituted or unsubstituted spirocyclic heterocycloalkyl. In embodiments, R⁹ is an R¹¹-substituted or unsubstituted C₆-C₁₂ spirocyclic cycloalkyl or R¹¹-substituted or unsubstituted 6 to 12 membered spirocyclic heterocycloalkyl. In embodiments, R⁹ is an R¹¹-substituted or unsubstituted bridged cycloalkyl or R¹¹-substituted or unsubstituted bridged heterocycloalkyl. In embodiments, R⁹ is an R¹¹-substituted or unsubstituted C₅-C₁₂ bridged cycloalkyl or R¹¹-substituted or unsubstituted 5 to 12 membered spirocyclic heterocycloalkyl.

In embodiments, R⁹ is

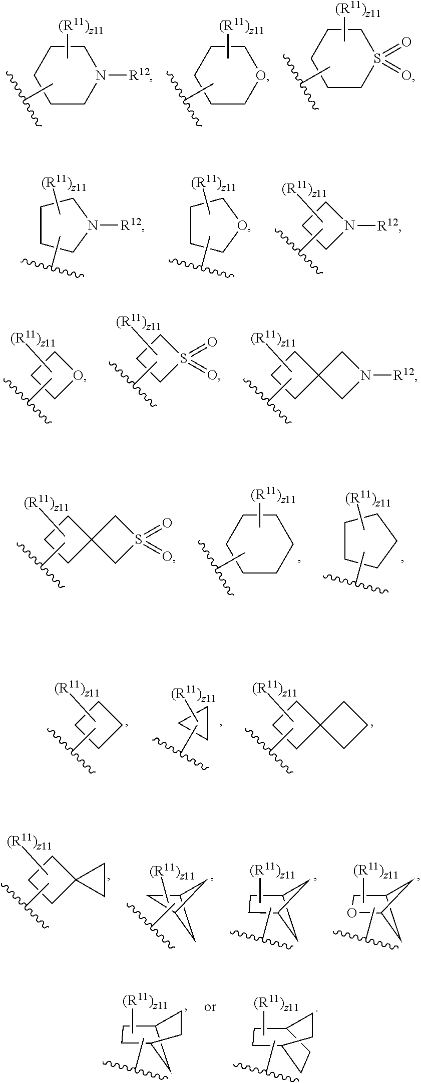

R¹¹ is as described herein, including in embodiments.

R¹² is hydrogen, halogen, —CX¹²₃, —CHX¹²₂, —CH₂X¹², —OCX¹²₃, —OCH₂X¹², —OCHX¹²₂, —SOₙ₁₂R^{12D}, —SOᵥ₁₂NR^{12A}R^{12B}, —C(O)R^{12C}, —C(O)OR^{12C}, —C(O)NR^{12A}R^{12B}, —OR^{12D}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{12A}, R^{12B}, R^{12C}, and R^{12D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{12A} and R^{12B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹² is independently —F, —Cl, —Br, or —I.

The symbol n12 is independently an integer from 0 to 4.

The symbol v12 is independently 1 or 2.

The symbol z11 is an integer from 0 to 13.

In embodiments, R⁹ is

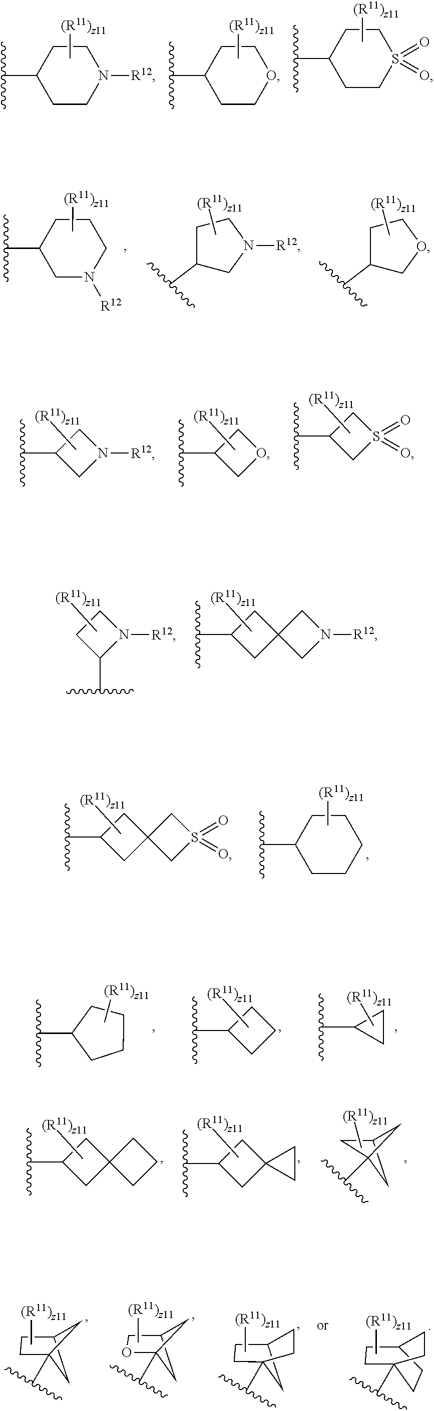

R¹¹, z11, and R¹² are as described herein, including in embodiments.

In embodiments, R⁹ is

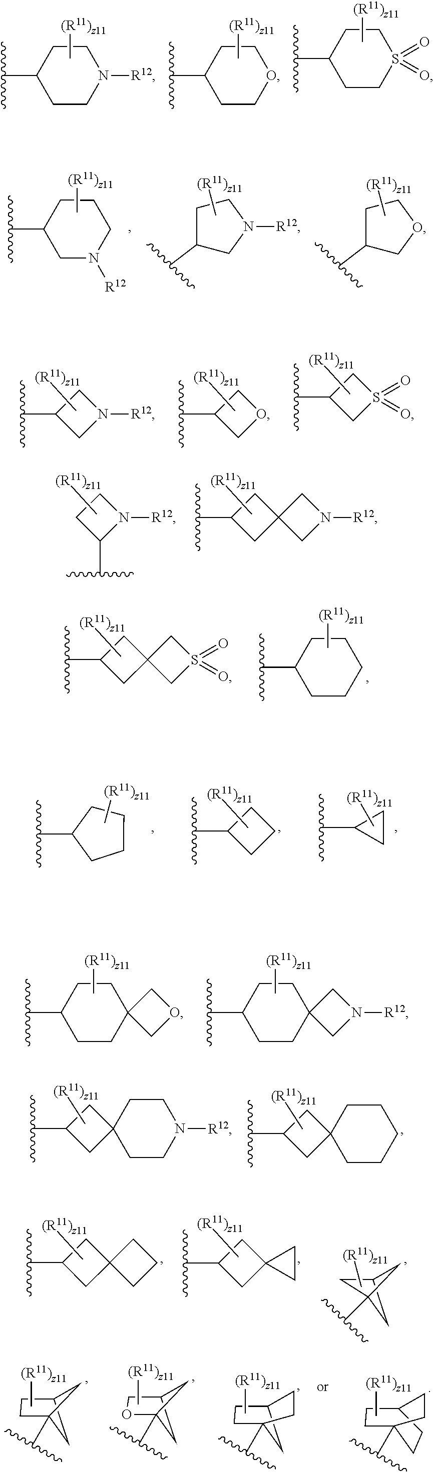

R¹¹, z11, and R² are as described herein, including in embodiments.

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

In embodiments, a substituted R^{11A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11A} is substituted with a plurality of groups selected from substituent groups, size-limited substituent groups, and lower substituent groups; each substituent group, size-limited substituent group, and/or lower substituent group may optionally be different. In embodiments, when R^{11A} is substituted, it is substituted with at least one substituent group. In embodiments, when R^{11A} is substituted, it is substituted with at least one size-limited substituent group. In embodiments, when R^{11A} is substituted, it is substituted with at least one lower substituent group.

In embodiments, a substituted R^{11B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11B} is substituted with a plurality of groups selected from substituent groups, size-limited substituent groups, and lower substituent groups; each substituent group, size-limited substituent group, and/or lower substituent group may optionally be different. In embodiments, when R^{11B} is substituted, it is substituted with at least one substituent group. In embodiments, when R^{11B} is substituted, it is substituted with at least one size-limited substituent group. In embodiments, when R^{11B} is substituted, it is substituted with at least one lower substituent group.

In embodiments, a substituted ring formed when R^{11A} and R^{11B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{11A} and R^{11B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{11A} and R^{11B} substituents bonded to the same nitrogen atom are joined is substituted, it is substituted with at least one substituent group. In embodiments, when the substituted ring formed when R^{11A} and R^{11B} substituents bonded to the same nitrogen atom are joined is substituted, it is substituted with at least one size-limited substituent group. In embodiments, when the substituted ring formed when R^{11A} and R^{11B} substituents bonded to the same nitrogen atom are joined is substituted, it is substituted with at least one lower substituent group.

In embodiments, a substituted R^{11C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11C} is substituted with a plurality of groups selected from substituent groups, size-limited substituent groups, and lower substituent groups; each substituent group, size-limited substituent group, and/or lower substituent group may optionally be different. In embodiments, when R^{11C} is substituted, it is substituted with at least one substituent group. In embodiments, when R^{11C} is substituted, it is substituted with at least one size-limited substituent group. In embodiments, when R^{11C} is substituted, it is substituted with at least one lower substituent group.

In embodiments, a substituted R^{11D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11D} is substituted with a plurality of groups selected from substituent groups, size-limited substituent groups, and lower substituent groups; each substituent group, size-limited substituent group, and/or lower substituent group may optionally be different. In embodiments, when R^{11D} is substituted, it is substituted with at least one substituent group. In embodiments, when R^{11D} is substituted, it is substituted with at least one size-limited substituent group. In embodiments, when R^{11D} is substituted, it is substituted with at least one lower substituent group.

In embodiments, R¹¹ is independently oxo, halogen, —CX¹¹₃, —CHX¹¹₂, —CH₂X¹¹, —OCX¹¹₃, —OCH₂X¹¹, —OCHX¹¹₂, —CN, —SO₁₁R^{11D}, —SOᵥ₁₁NR^{11A}R^{11B}, —NHC(O)NR^{11A}R^{11B}, —NR^{11A}R^{11B}, —C(O)R^{11C}, —C(O)OR^{11C}, —C(O)NR^{11A}R^{11B}, —OR^{11D}, —SR^{11D}, —NR^{11A}SO₂R^{11D}, —NR^{11A}C(O)R^{11C}, —NR^{11A}C(O)OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹¹ is independently oxo, halogen, —CX¹¹₃, —CN, —C(O)OR^{11C}, —C(O)NR^{11A}R^{11B}, —C(O)R^{11C}, —OR^{11D}, substituted or unsubstituted C₁-C₆ alkyl, or substituted or unsubstituted 2 to 5 membered heteroalkyl.

In embodiments, R¹¹ is independently oxo, halogen, —CX¹¹₃, —CHX¹¹₂, —CN, —SOₙ₁₁R^{11D}, —C(O)R^{11C}, —C(O)OR^{11C}, —C(O)NR^{11A}R^{11B}, —C(O)R^{11C}, —OR^{11D}, —NR^{11A}SO₂R^{11D}, —NR^{11A}C(O)R^{11C}, —NR^{11A}C(O)OR^{11C} substituted or unsubstituted C₁-C₆ alkyl, substituted or unsubstituted 2 to 5 membered heteroalkyl, or substituted or unsubstituted 5 to 10 membered heteroaryl.

In embodiments, R^{11A} is independently hydrogen or unsubstituted C₁-C₆ alkyl; and R^{11B} is independently hydrogen. In embodiments, R^{11A} is independently hydrogen. In embodiments, R^{11A} is independently unsubstituted C₁-C₆ alkyl. In embodiments, R^{11A} is independently unsubstituted methyl. In embodiments, R^{11A} is independently unsubstituted ethyl. In embodiments, R^{11A} is independently unsubstituted propyl. In embodiments, R^{11A} is independently unsubstituted n-propyl. In embodiments, R^{11A} is independently unsubstituted isopropyl. In embodiments, R^{11A} is independently unsubstituted butyl. In embodiments, R^{11A} is independently unsubstituted n-butyl. In embodiments, R^{11A} is independently unsubstituted isobutyl. In embodiments, R^{11A} is independently unsubstituted tert-butyl. In embodiments, R^{11A} is independently unsubstituted pentyl. In embodiments, R^{11A} is independently unsubstituted hexyl. In embodiments, R^{11A} is independently —OH. In embodiments, R^{11A} is independently —C(O)CH₃. In embodiments, R^{11B} is independently hydrogen.

In embodiments, R^{11C} is independently hydrogen or unsubstituted C₁-C₆ alkyl. In embodiments, R^{11C} is independently hydrogen. In embodiments, R^{11C} is independently unsubstituted C₁-C₆ alkyl. In embodiments, R^{11C} is independently unsubstituted methyl. In embodiments, R^{11C} is independently unsubstituted ethyl. In embodiments, R^{11C} is independently unsubstituted propyl. In embodiments, R^{11C} is independently unsubstituted n-propyl. In embodiments, R^{11C} is independently unsubstituted isopropyl. In embodiments, R^{11C} is independently unsubstituted butyl. In embodiments, R^{11C} is independently unsubstituted n-butyl. In embodiments, R^{11C} is independently unsubstituted isobutyl. In embodiments, R^{11C} is independently unsubstituted tert-butyl. In embodiments, R^{11C} is independently unsubstituted pentyl. In embodiments, R^{11C} is independently unsubstituted hexyl. In embodiments, R^{11C} is independently substituted or unsubstituted 5 to 10 membered heteroaryl. In embodiments, R^{11C} is independently unsubstituted thiazolyl. In embodiments, R^{11C} is independently

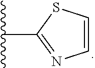 [SMILES: *c1nccs1]

In embodiments, R^{11D} is independently hydrogen or unsubstituted C₁-C₆ alkyl. In embodiments, R^{11D} is independently hydrogen. In embodiments, R^{11D} is independently unsubstituted C₁-C₆ alkyl. In embodiments, R^{11D} is independently unsubstituted methyl. In embodiments, R^{11D} is independently unsubstituted ethyl. In embodiments, R^{11D} is independently unsubstituted propyl. In embodiments, R^{11D} is independently unsubstituted n-propyl. In embodiments, R^{11D} is independently unsubstituted isopropyl. In embodiments, R^{11D} is independently unsubstituted butyl. In embodiments, R^{11D} is independently unsubstituted n-butyl. In embodiments, R^{11D} is independently unsubstituted isobutyl. In embodiments, R^{11D} is independently unsubstituted tert-butyl. In embodiments, R^{11D} is independently unsubstituted pentyl. In embodiments, R^{11D} is independently unsubstituted hexyl.

In embodiments, two R¹¹ substituents are joined to form a substituted or unsubstituted cycloalkyl. In embodiments, two R¹¹ substituents are joined to form a substituted or unsubstituted C₃-C₈ cycloalkyl. In embodiments, two R¹¹ substituents are joined to form an unsubstituted cyclopropyl. In embodiments, two R¹¹ substituents are joined to form an unsubstituted cyclobutyl. In embodiments, two R¹¹ substituents are joined to form an unsubstituted cyclopentyl. In embodiments, two R¹¹ substituents are joined to form an unsubstituted cyclohexyl. In embodiments, two R¹¹ substituents are joined to form an unsubstituted cycloheptyl. In embodiments, two R¹¹ substituents are joined to form an unsubstituted cyclooctyl.

In embodiments, R¹¹ is independently oxo. In embodiments, R¹¹ is independently halogen. In embodiments, R¹¹ is —F. In embodiments, R¹¹ is independently —CF₃. In embodiments, R¹¹ is independently —CHF₂. In embodiments, R¹¹ is independently —CN. In embodiments, R¹¹ is independently —OH. In embodiments, R¹¹ is independently —C(O)OH.

In embodiments, R¹¹ is independently —C(O)OCH₃. In embodiments, R¹¹ is independently —C(O)OCH₂CH₃. In embodiments, R¹¹ is independently —C(O)CH₃. In embodiments, R¹¹ is independently —C(O)NH₂. In embodiments, R¹¹ is independently —C(O)NHOH. In embodiments, R¹¹ is independently —S(O)₂CH₃. In embodiments, R¹¹ is independently —NHC(O)CH₃. In embodiments, R¹¹ is independently —C(O)H. In embodiments, R¹¹ is independently —NHS(O)₂CH₃. In embodiments, R¹¹ is independently —C(O)NHS(O)₂CH₃. In embodiments, R¹¹ is independently unsubstituted C₁-C₆ alkyl. In embodiments, R¹¹ is independently unsubstituted methyl. In embodiments, R¹¹ is independently unsubstituted ethyl. In embodiments, R¹¹ is independently unsubstituted propyl. In embodiments, R¹¹ is independently unsubstituted n-propyl. In embodiments, R¹¹ is independently unsubstituted isopropyl. In embodiments, R¹¹ is independently unsubstituted butyl. In embodiments, R¹¹ is independently unsubstituted n-butyl. In embodiments, R¹¹ is independently unsubstituted isobutyl. In embodiments, R¹¹ is independently unsubstituted tert-butyl. In embodiments, R¹¹ is independently unsubstituted pentyl. In embodiments, R¹¹ is independently unsubstituted hexyl. In embodiments, R¹¹ is independently substituted C₁-C₆ alkyl. In embodiments, R¹¹ is independently substituted methyl. In embodiments, R¹¹ is independently substituted ethyl. In embodiments, R¹¹ is independently substituted propyl. In embodiments, R¹¹ is independently substituted n-propyl.
In embodiments, R¹¹ is independently substituted isopropyl. In embodiments, R¹¹ is independently substituted butyl. In embodiments, R¹¹ is independently substituted n-butyl.
In embodiments, R¹¹ is independently substituted isobutyl. In embodiments, R¹¹ is independently substituted tert-butyl. In embodiments, R¹¹ is independently substituted pentyl. In embodiments, R¹¹ is independently substituted hexyl. In embodiments, R¹¹ is independently substituted or unsubstituted 2 to 6 membered heteroalkyl. In embodiments, R¹¹ is independently unsubstituted methoxy. In embodiments, R¹¹ is independently unsubstituted ethoxy. In embodiments, R¹¹ is independently unsubstituted propoxy. In embodiments, R¹¹ is independently unsubstituted n-propoxy. In embodiments, R¹¹ is independently unsubstituted isopropoxy. In embodiments, R¹¹ is independently unsubstituted butoxy. In embodiments, R¹¹ is independently

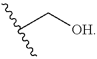 [SMILES: *CO]

In embodiments, R¹¹ is independently

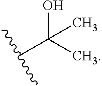 [SMILES: *C(C)(C)O]

In embodiments, R¹¹ is independently

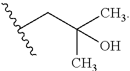 [SMILES: *CC(C)(C)O]

In embodiments, R¹¹ is independently

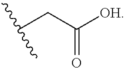 [SMILES: *CC(=O)O]

In embodiments, R¹¹ is independently

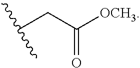 [SMILES: *CC(=O)OC]

In embodiments, R¹¹ is independently

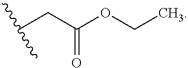 [SMILES: *CC(=O)OCC]

In embodiments, R¹¹ is independently

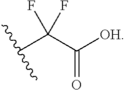 [SMILES: *C(F)(F)C(=O)O]

In embodiments, R¹¹ is independently

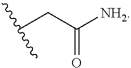 [SMILES: *CC(N)=O]

In embodiments, R¹¹ is independently

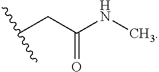 [SMILES: *CC(=O)NC]

In embodiments, R¹¹ is independently

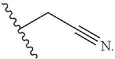 [SMILES: *CC#N]

In embodiments, R¹¹ is independently

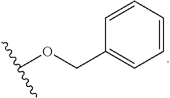 [SMILES: *OCc1ccccc1]

In embodiments, R¹¹ is independently

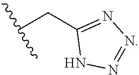 [SMILES: *Cc1nnn[nH]1]

In embodiments, R¹¹ is independently

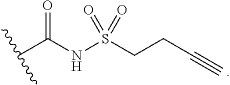 [SMILES: *C(=O)NS(=O)(=O)CCC#C]

In embodiments, R¹¹ is independently

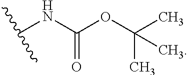 [SMILES: *NC(=O)OC(C)(C)C]

In embodiments, R¹¹ is independently

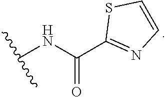 [SMILES: *NC(=O)c1nccs1]

In embodiments, R¹¹ is independently

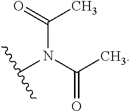 [SMILES: *N(C(C)=O)C(C)=O]

In embodiments, R¹¹ is independently substituted or unsubstituted 5 to 10 membered heteroaryl. In embodiments, R¹¹ is independently unsubstituted pyridyl. In embodiments, R¹¹ is independently unsubstituted 2-pyridyl. In embodiments, R¹¹ is independently unsubstituted 3-pyridyl. In embodiments, R¹¹ is independently unsubstituted 4-pyridyl. In embodiments, R¹¹ is independently unsubstituted oxazolyl. In embodiments, R¹¹ is independently unsubstituted isoxazolyl. In embodiments, R¹¹ is independently substituted isoxazolyl. In embodiments, R¹¹ is independently

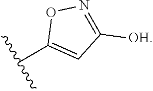 [SMILES: *c1cc(O)no1]

In embodiments, R¹¹ is independently unsubstituted tetrazolyl.

In embodiments, z11 is 0. In embodiments, z11 is 1. In embodiments, z11 is 2. In embodiments, z11 is 3. In embodiments, z11 is 4. In embodiments, z11 is 5. In embodiments, z11 is 6. In embodiments, z11 is 7. In embodiments, z11 is 8. In embodiments, z11 is 9. In embodiments, z11 is 10. In embodiments, z11 is 11. In embodiments, z11 is 12. In embodiments, z11 is 13.

In embodiments, a substituted R¹² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹² is substituted with a plurality of groups selected from substituent groups, size-limited substituent groups, and lower substituent groups; each substituent group, size-limited substituent group, and/or lower substituent group may optionally be different. In embodiments, when R¹² is substituted, it is substituted with at least one substituent group. In embodiments, when R¹² is substituted, it is substituted with at least one size-limited substituent group. In embodiments, when R¹² is substituted, it is substituted with at least one lower substituent group.

In embodiments, a substituted R^{12A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12A} is substituted with a plurality of groups selected from substituent groups, size-limited substituent groups, and lower substituent groups; each substituent group, size-limited substituent group, and/or lower substituent group may optionally be different. In embodiments, when R^{12A} is substituted, it is substituted with at least one substituent group. In embodiments, when R^{12A} is substituted, it is substituted with at least one size-limited substituent group. In embodiments, when R^{12A} is substituted, it is substituted with at least one lower substituent group.

In embodiments, a substituted R^{12B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12B} is substituted with a plurality of groups selected from substituent groups, size-limited substituent groups, and lower substituent groups; each substituent group, size-limited substituent group, and/or lower substituent group may optionally be different. In embodiments, when R^{12B} is substituted, it is substituted with at least one substituent group. In embodiments, when R^{12B} is substituted, it is substituted with at least one size-limited substituent group. In embodiments, when R^{12B} is substituted, it is substituted with at least one lower substituent group.

In embodiments, a substituted ring formed when R^{12A} and R^{12B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{12A} and R^{12B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{12A} and R^{12B} substituents bonded to the same nitrogen atom are joined is substituted, it is substituted with at least one substituent group. In embodiments, when the substituted ring formed when R^{12A} and R^{12B} substituents bonded to the same nitrogen atom are joined is substituted, it is substituted with at least one size-limited substituent group. In embodiments, when the substituted ring formed when R^{12A} and R^{12B} substituents bonded to the same nitrogen atom are joined is substituted, it is substituted with at least one lower substituent group.

In embodiments, a substituted R^{12C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12C} is substituted with a plurality of groups selected from substituent groups, size-limited substituent groups, and lower substituent groups; each substituent group, size-limited substituent group, and/or lower substituent group may optionally be different. In embodiments, when R^{12C} is substituted, it is substituted with at least one substituent group. In embodiments, when R^{12C} is substituted, it is substituted with at least one size-limited substituent group. In embodiments, when R^{12C} is substituted, it is substituted with at least one lower substituent group.

In embodiments, a substituted R^{12D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12D} is substituted with a plurality of groups selected from substituent groups, size-limited substituent groups, and lower substituent groups; each substituent group, size-limited substituent group, and/or lower substituent group may optionally be different. In embodiments, when R^{12D} is substituted, it is substituted with at least one substituent group. In embodiments, when R^{12D} is substituted, it is substituted with at least one size-limited substituent group. In embodiments, when R^{12D} is substituted, it is substituted with at least one lower substituent group.

In embodiments, R¹² is hydrogen, halogen, —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NHC(O)NR^{12A}R^{12B}, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —C(O)H, —C(O)OH, —CONH₂, —OH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹² is hydrogen, —C(O)R^{12C}, —SOₙ₁₂R^{12D}, —SOᵥ₁₂NR^{12A}R^{12B}, —C(O)OR^{12C}, —C(O)NR^{12A}R^{12B}, unsubstituted C₁-C₆ alkyl, unsubstituted C₃-C₈ cycloalkyl, or unsubstituted 3 to 8 membered heterocycloalkyl.

In embodiments, R^{12A} is hydrogen, unsubstituted C₁-C₆ alkyl, or unsubstituted C₃-C₈ cycloalkyl; and R^{12B} is hydrogen. In embodiments, R^{12A} is hydrogen. In embodiments, R^{12A} is unsubstituted C₁-C₆ alkyl. In embodiments, R^{12A} is unsubstituted methyl. In embodiments, R^{12A} is unsubstituted ethyl. In embodiments, R^{12A} is unsubstituted propyl. In embodiments, R^{12A} is unsubstituted n-propyl. In embodiments, R^{12A} is unsubstituted isopropyl. In embodiments, R^{12A} is unsubstituted butyl. In embodiments, R^{12A} is unsubstituted n-butyl. In embodiments, R^{12A} is unsubstituted isobutyl. In embodiments, R^{12A} is unsubstituted tert-butyl. In embodiments, R^{12A} is unsubstituted pentyl. In embodiments, R^{12A} is unsubstituted hexyl. In embodiments, R^{12A} is unsubstituted C₃-C₈ cycloalkyl. In embodiments, R^{12A} is unsubstituted cyclopropyl. In embodiments, R^{12A} is unsubstituted cyclobutyl. In embodiments, R^{12A} is unsubstituted cyclopentyl. In embodiments, R^{12A} is unsubstituted cyclohexyl. In embodiments, R^{12B} is hydrogen.

In embodiments, R^{12C} is substituted or unsubstituted C₁-C₆ alkyl, substituted or unsubstituted 2 to 6 membered heteroalkyl, or substituted or unsubstituted C₃-C₈ cycloalkyl.

In embodiments, R^{12C} is hydrogen. In embodiments, R^{12C} is unsubstituted C₁-C₆ alkyl. In embodiments, R^{12C} is unsubstituted methyl. In embodiments, R^{12C} is unsubstituted ethyl. In embodiments, R^{12C} is unsubstituted propyl. In embodiments, R^{12C} is unsubstituted n-propyl.
In embodiments, R^{12C} is unsubstituted isopropyl. In embodiments, R^{12C} is unsubstituted butyl. In embodiments, R^{12C} is unsubstituted n-butyl. In embodiments, R^{12C} is unsubstituted isobutyl. In embodiments, R^{12C} is unsubstituted tert-butyl. In embodiments, R^{12C} is unsubstituted pentyl. In embodiments, R^{12C} is unsubstituted hexyl. In embodiments, R^{12C} is unsubstituted C₃-C₈ cycloalkyl. In embodiments, R^{12C} is unsubstituted cyclopropyl. In embodiments, R^{12C} is unsubstituted cyclobutyl. In embodiments, R^{12C} is unsubstituted cyclopentyl. In embodiments, R^{12C} is unsubstituted cyclohexyl.

In embodiments, R^{12D} is unsubstituted C₁-C₆ alkyl. In embodiments, R^{12D} is hydrogen. In embodiments, R^{12D} is unsubstituted C₁-C₆ alkyl. In embodiments, R^{12D} is unsubstituted methyl. In embodiments, R^{12D} is unsubstituted ethyl. In embodiments, R^{12D} is unsubstituted propyl. In embodiments, R^{12D} is unsubstituted n-propyl. In embodiments, R^{12D} is unsubstituted isopropyl. In embodiments, R^{12D} is unsubstituted butyl. In embodiments, R^{12D} is unsubstituted n-butyl. In embodiments, R^{12D} is unsubstituted isobutyl. In embodiments, R^{12D} is unsubstituted tert-butyl. In embodiments, R^{12D} is unsubstituted pentyl.

In embodiments, R^{12D} is unsubstituted hexyl.

In embodiments, R⁹ is

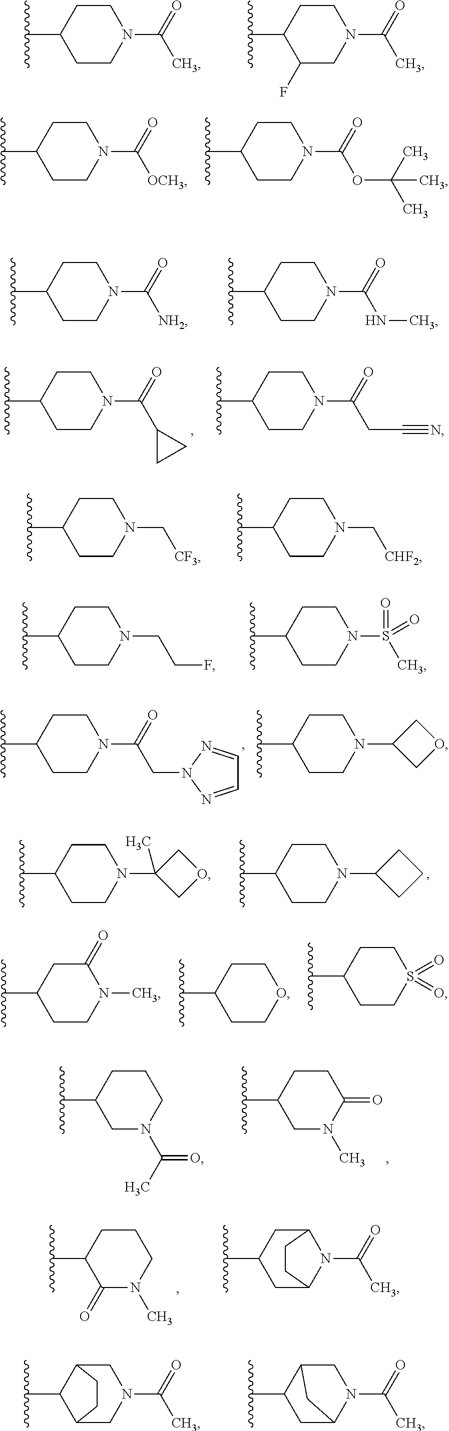

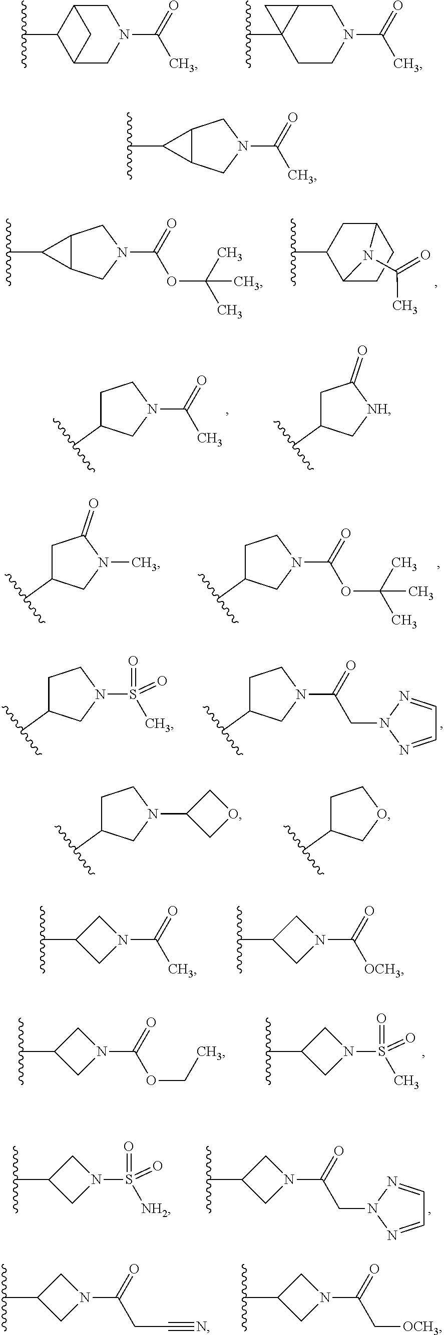

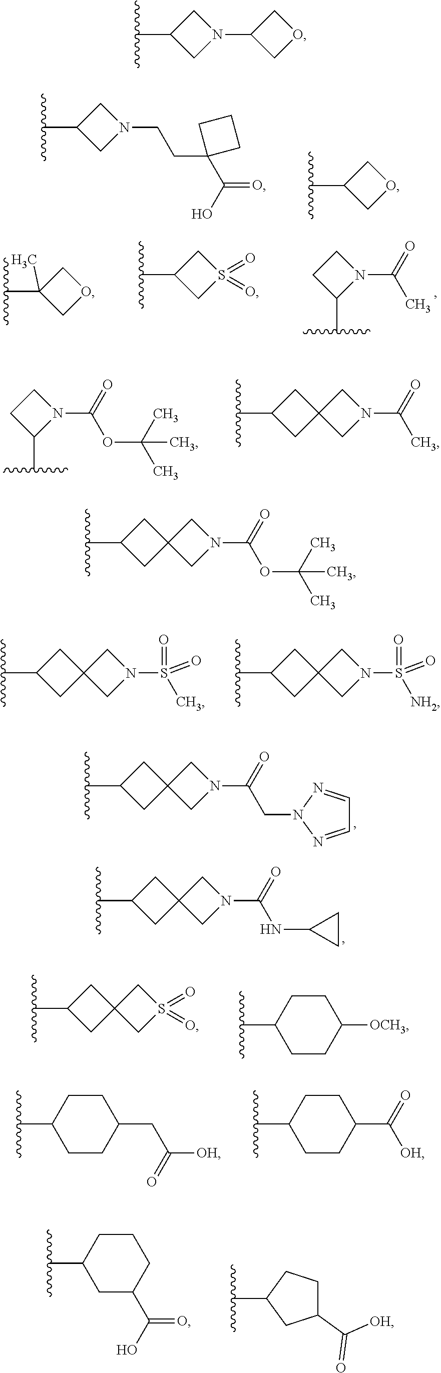

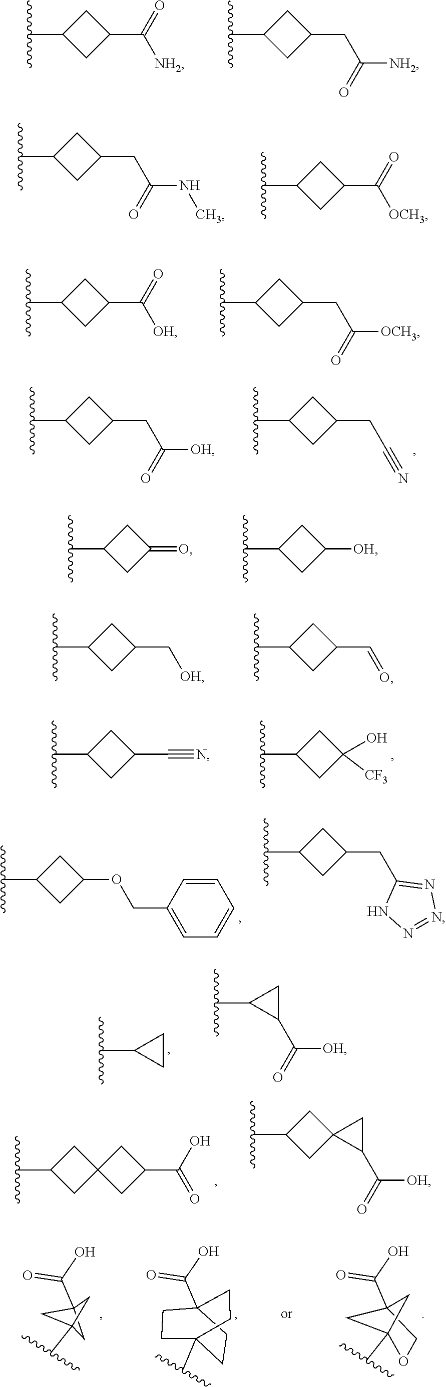

In embodiments, R⁹ is

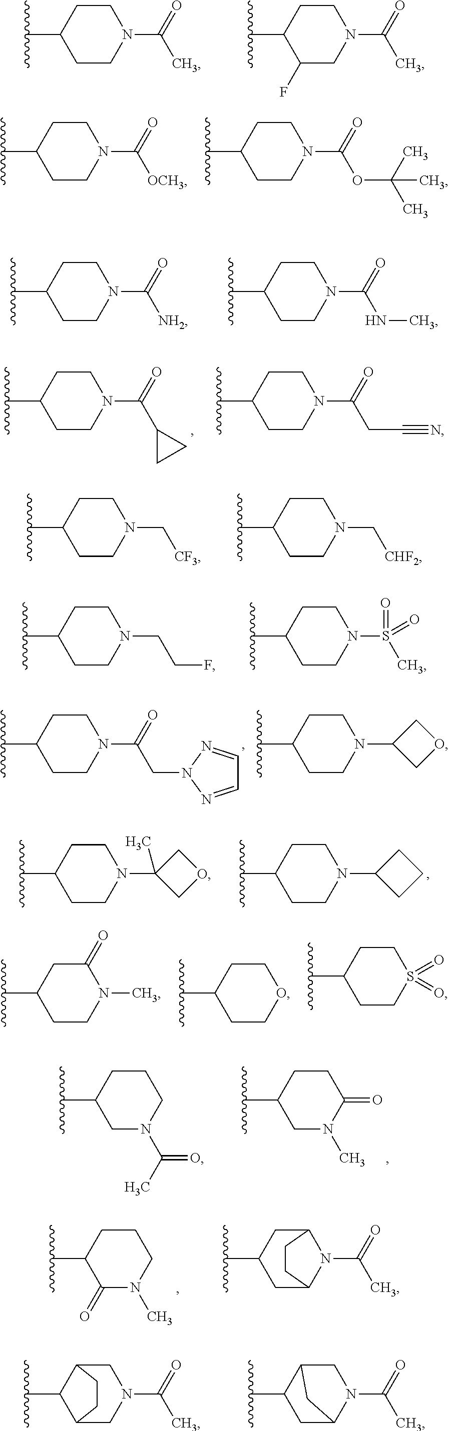

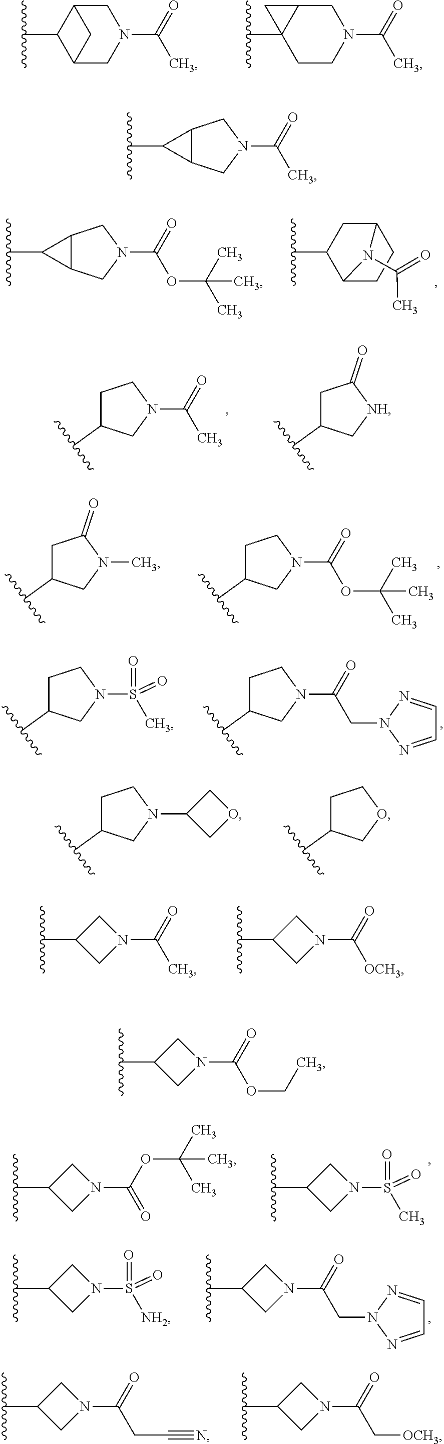

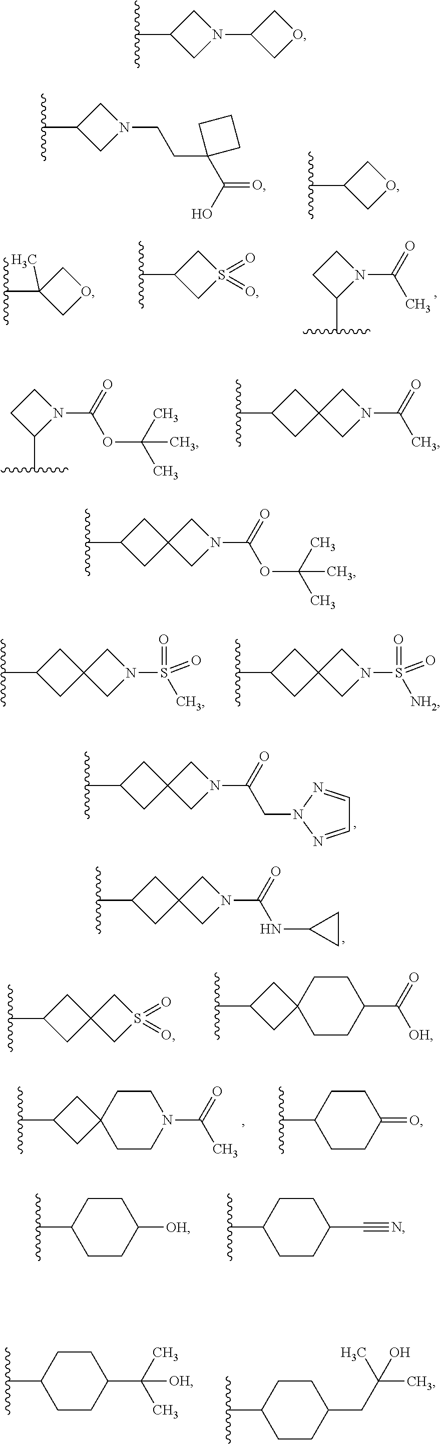

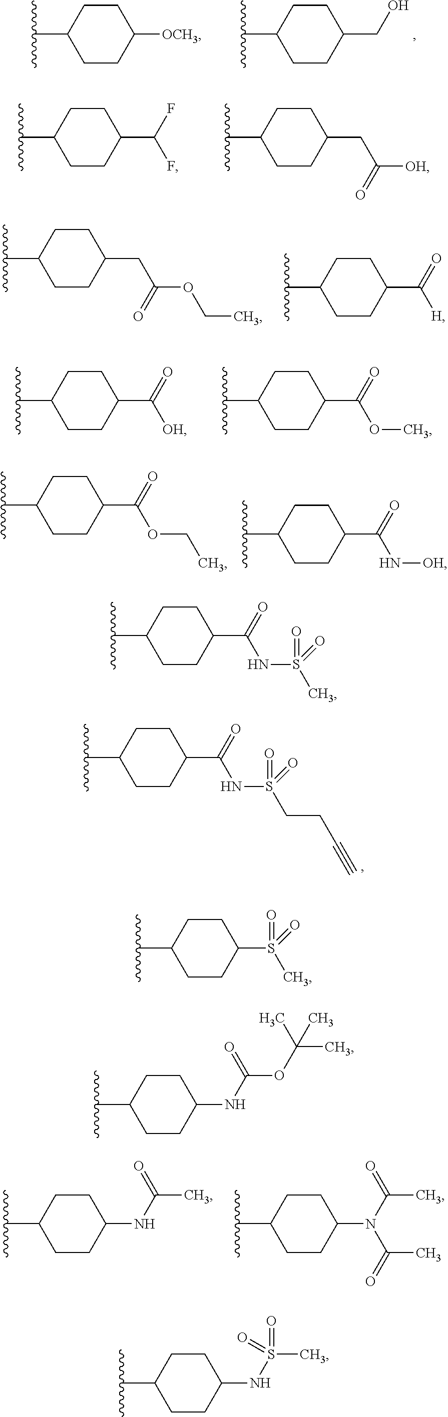

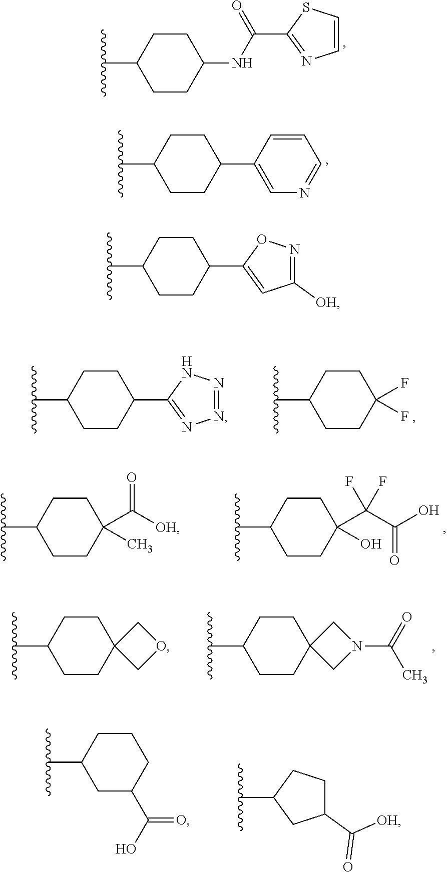

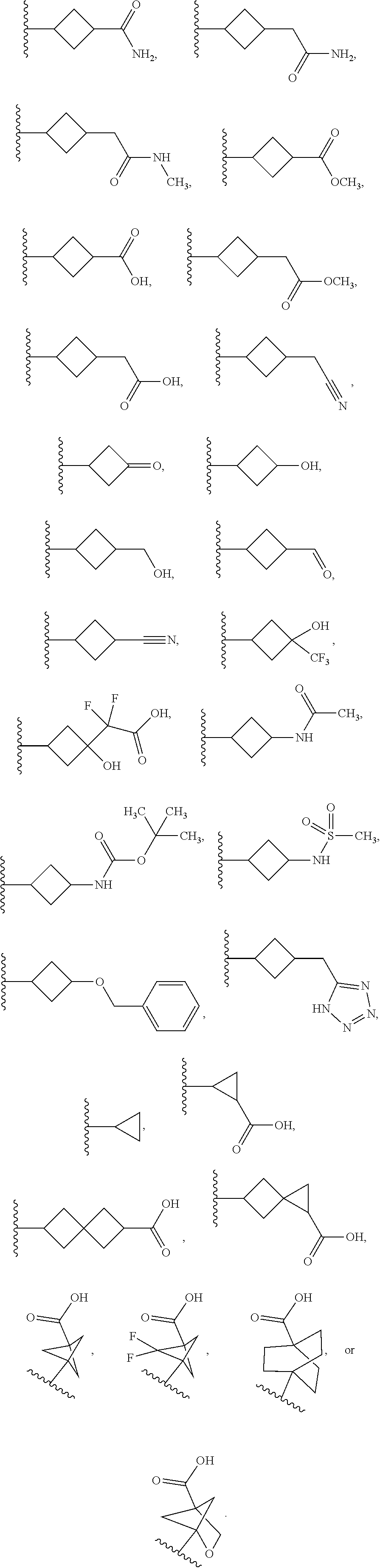

In embodiments, R⁹ is

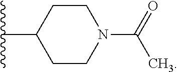 [SMILES: *C1CCN(C(C)=O)CC1]

In embodiments, R⁹ is

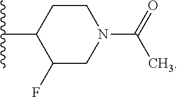 [SMILES: *C1CCN(C(C)=O)CC1F]

In embodiments, R⁹ is

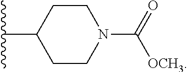 [SMILES: *C1CCN(C(=O)OC)CC1]

In embodiments, R⁹ is

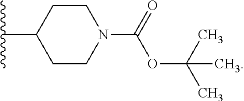 [SMILES: *C1CCN(C(=O)OC(C)(C)C)CC1]

In embodiments, R⁹ is

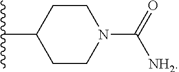 [SMILES: *C1CCN(C(N)=O)CC1]

In embodiments, R⁹ is

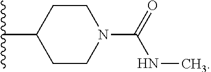 [SMILES: *C1CCN(C(=O)NC)CC1]

In embodiments, R⁹ is

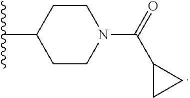 [SMILES: *C1CCN(C(=O)C2CC2)CC1]

In embodiments, R⁹ is

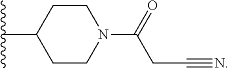 [SMILES: *C1CCN(C(=O)CC#N)CC1]

In embodiments, R⁹ is

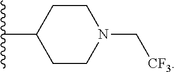 [SMILES: *C1CCN(CC(F)(F)F)CC1]

In embodiments, R⁹ is

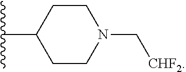 [SMILES: *C1CCN(CC(F)F)CC1]

In embodiments, R⁹ is

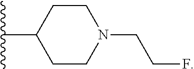 [SMILES: *C1CCN(CCF)CC1]

In embodiments, R⁹ is

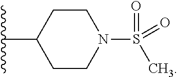 [SMILES: *C1CCN(S(C)(=O)=O)CC1]

In embodiments, R⁹ is

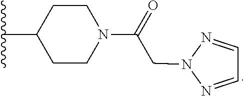 [SMILES: *C1CCN(C(=O)Cn2nccn2)CC1]

In embodiments, R⁹ is

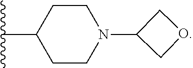 [SMILES: *C1CCN(C2COC2)CC1]

In embodiments, R⁹ is

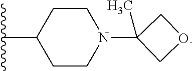 [SMILES: *C1CCN(C2(C)COC2)CC1]

In embodiments, R⁹ is

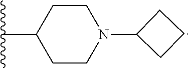 [SMILES: *C1CCN(C2CCC2)CC1]

In embodiments, R⁹ is

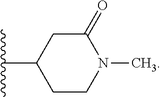 [SMILES: *C1CCN(C)C(=O)C1]

In embodiments, R⁹ is

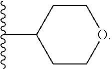 [SMILES: *C1CCOCC1]

In embodiments, R⁹ is

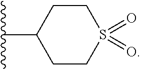 [SMILES: *C1CCS(=O)(=O)CC1]

In embodiments, R⁹ is

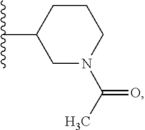 [SMILES: *C1CCCN(C(C)=O)C1]

In embodiments, R⁹ is

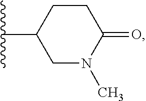 [SMILES: *C1CCC(=O)N(C)C1]

In embodiments, R⁹ is

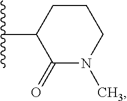 [SMILES: *C1CCCN(C)C1=O]

In embodiments, R⁹ is

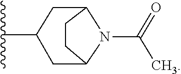 [SMILES: *C1CC2CCC(C1)N2C(C)=O]

In embodiments, R⁹ is

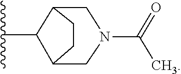 [SMILES: *C1C2CCC1CN(C(C)=O)C2]

In embodiments, R⁹ is

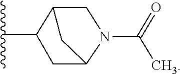 [SMILES: *C1CC2CC1CN2C(C)=O]

In embodiments, R⁹ is

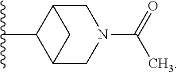 [SMILES: *C1C2CC1CN(C(C)=O)C2]

In embodiments, R⁹ is

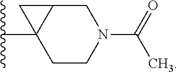 [SMILES: *C12CCN(C(C)=O)CC1C2]

In embodiments, R⁹ is

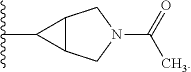 [SMILES: *C1C2CN(C(C)=O)CC12]

In embodiments, R⁹ is

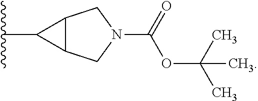 [SMILES: *C1C2CN(C(=O)OC(C)(C)C)CC12]

In embodiments, R⁹ is

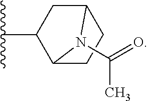 [SMILES: *C1CC2CCC1N2C(C)=O]

In embodiments, R⁹ is

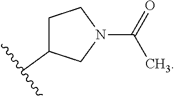 [SMILES: *C1CCN(C(C)=O)C1]

In embodiments, R⁹ is

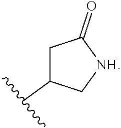 [SMILES: *C1CNC(=O)C1]

In embodiments, R⁹ is

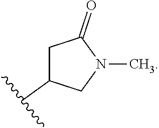 [SMILES: *C1CC(=O)N(C)C1]

In embodiments, R⁹ is

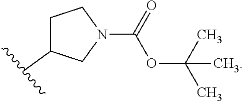 [SMILES: *C1CCN(C(=O)OC(C)(C)C)C1]

In embodiments, R⁹ is

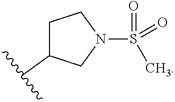 [SMILES: *C1CCN(S(C)(=O)=O)C1]

In embodiments, R⁹ is

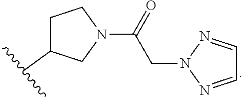 [SMILES: *C1CCN(C(=O)Cn2nccn2)C1]

In embodiments, R⁹ is

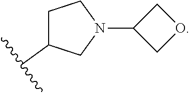 [SMILES: *C1CCN(C2COC2)C1]

In embodiments, R⁹ is

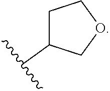 [SMILES: *C1CCOC1]

In embodiments, R⁹ is

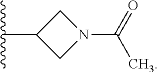 [SMILES: *C1CN(C(C)=O)C1]

In embodiments, R⁹ is

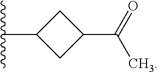 [SMILES: *C1CC(C(C)=O)C1]

In embodiments, R⁹ is

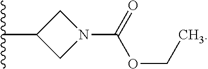 [SMILES: *C1CN(C(=O)OCC)C1]

In embodiments, R⁹ is

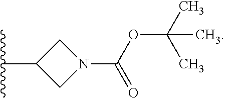 [SMILES: *C1CN(C(=O)OC(C)(C)C)C1]

In embodiments, R⁹ is

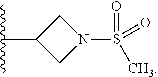 [SMILES: *C1CN(S(C)(=O)=O)C1]

In embodiments, R⁹ is

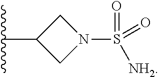 [SMILES: *C1CN(S(N)(=O)=O)C1]

In embodiments, R⁹ is

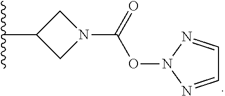 [SMILES: *C1CN(C(=O)On2nccn2)C1]

In embodiments, R⁹ is

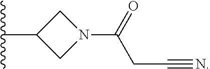 [SMILES: *C1CN(C(=O)CC#N)C1]

In embodiments, R⁹ is

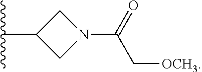 [SMILES: *C1CN(C(=O)COC)C1]

In embodiments, R⁹ is

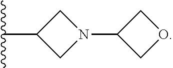 [SMILES: *C1CN(C2COC2)C1]

In embodiments, R⁹ is

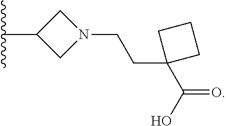 [SMILES: *C1CN(CCC2(C(=O)O)CCC2)C1]

In embodiments, R⁹ is

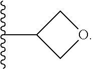 [SMILES: *C1COC1]

In embodiments, R⁹ is

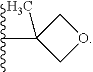 [SMILES: *C1(C)COC1]

In embodiments, R⁹ is

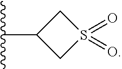 [SMILES: *C1CS(=O)(=O)C1]

In embodiments, R⁹ is

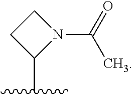 [SMILES: *C1CCN1C(C)=O]

In embodiments, R⁹ is

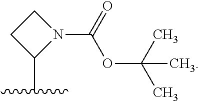 [SMILES: *C1CCN1C(=O)OC(C)(C)C]

In embodiments, R⁹ is

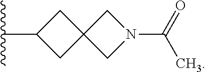 [SMILES: *C1CC2(C1)CN(C(C)=O)C2]

In embodiments, R⁹ is

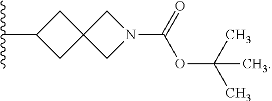 [SMILES: *C1CC2(C1)CN(C(=O)OC(C)(C)C)C2]

In embodiments, R⁹ is

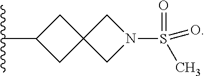 [SMILES: *C1CC2(C1)CN(S(C)(=O)=O)C2]

In embodiments, R⁹ is

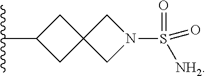 [SMILES: *C1CC2(C1)CN(S(N)(=O)=O)C2]

In embodiments, R⁹ is

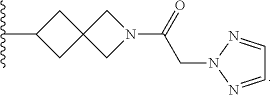 [SMILES: *C1CC2(C1)CN(C(=O)Cn1nccn1)C2]

In embodiments, R⁹ is

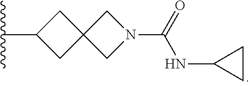 [SMILES: *C1CC2(C1)CN(C(=O)NC1CC1)C2]

In embodiments, R⁹ is

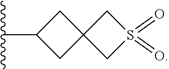 [SMILES: *C1CC2(C1)CS(=O)(=O)C2]

In embodiments, R⁹ is

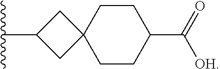 [SMILES: *C1CC2(CCC(C(=O)O)CC2)C1]

In embodiments, R⁹ is

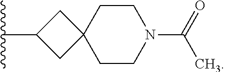 [SMILES: *C1CC2(CCN(C(C)=O)CC2)C1]

In embodiments, R⁹ is

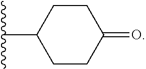 [SMILES: *C1CCC(=O)CC1]

In embodiments, R⁹ is

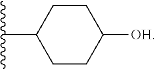 [SMILES: *C1CCC(O)CC1]

In embodiments, R⁹ is

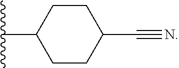 [SMILES: *C1CCC(C#N)CC1]

In embodiments, R⁹ is

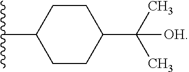 [SMILES: *C1CCC(C(C)(C)O)CC1]

In embodiments, R⁹ is

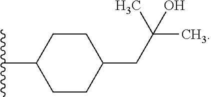 [SMILES: *C1CCC(CC(C)(C)O)CC1]

In embodiments, R⁹ is

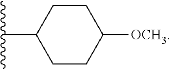 [SMILES: *C1CCC(OC)CC1]

In embodiments, R⁹ is

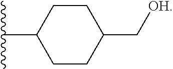 [SMILES: *C1CCC(CO)CC1]

In embodiments, R⁹ is

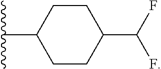 [SMILES: *C1CCC(C(F)F)CC1]

In embodiments, R⁹ is

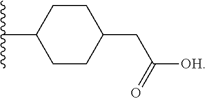 [SMILES: *C1CCC(CC(=O)O)CC1]

In embodiments, R⁹ is

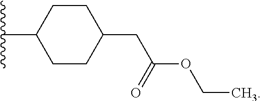 [SMILES: *C1CCC(CC(=O)OCC)CC1]

In embodiments, R⁹ is

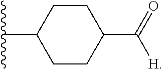

In embodiments, R⁹ is

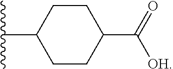 [SMILES: *C1CCC(C(=O)O)CC1]

In embodiments, R⁹ is

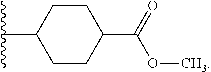 [SMILES: *C1CCC(C(=O)OC)CC1]

In embodiments, R⁹ is

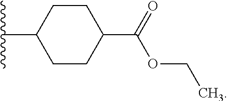 [SMILES: *C1CCC(C(=O)OCC)CC1]

In embodiments, R⁹ is

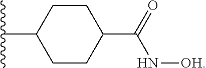 [SMILES: *C1CCC(C(=O)NO)CC1]

In embodiments, R⁹ is

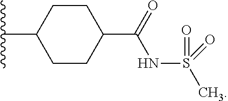 [SMILES: *C1CCC(C(=O)NS(C)(=O)=O)CC1]

In embodiments, R⁹ is

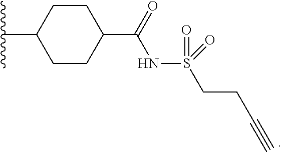 [SMILES: *C1CCC(C(=O)NS(=O)(=O)CCC#C)CC1]

In embodiments, R⁹ is

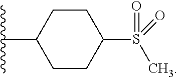 [SMILES: *C1CCC(S(C)(=O)=O)CC1]

In embodiments, R⁹ is

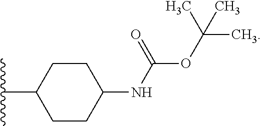 [SMILES: *C1CCC(NC(=O)OC(C)(C)C)CC1]

In embodiments, R⁹ is

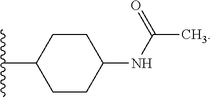 [SMILES: *C1CCC(NC(C)=O)CC1]

In embodiments, R⁹ is

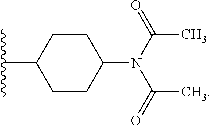 [SMILES: *C1CCC(N(C(C)=O)C(C)=O)CC1]

In embodiments, R⁹ is

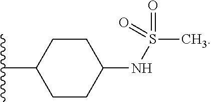 [SMILES: *C1CCC(NS(C)(=O)=O)CC1]

In embodiments, R⁹ is

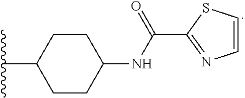 [SMILES: *C1CCC(NC(=O)c2nccs2)CC1 ; "."]

In embodiments, R⁹ is

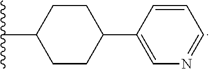 [SMILES: *C1CCC(c2cccnc2)CC1 ; "."]

In embodiments, R⁹ is

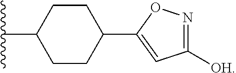 [SMILES: *C1CCC(c2cc(O)no2)CC1]

In embodiments, R⁹ is

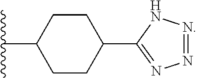 [SMILES: *C1CCC(c2nnn[nH]2)CC1]

In embodiments, R⁹ is

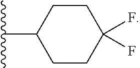 [SMILES: *C1CCC(F)(F)CC1]

In embodiments, R⁹ is

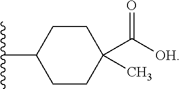 [SMILES: *C1CCC(C)(C(=O)O)CC1]

In embodiments, R⁹ is

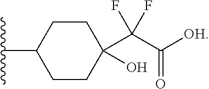 [SMILES: *C1CCC(O)(C(F)(F)C(=O)O)CC1]

In embodiments, R⁹ is

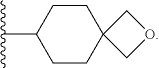 [SMILES: *C1CCC2(CC1)COC2]

In embodiments, R⁹ is

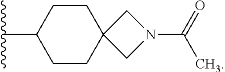 [SMILES: *C1CCC2(CC1)CN(C(C)=O)C2]

In embodiments, R⁹ is

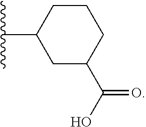 [SMILES: *C1CCCC(C(=O)O)C1]

In embodiments, R⁹ is

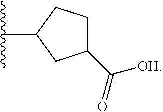 [SMILES: *C1CCC(C(=O)O)C1]

In embodiments, R⁹ is

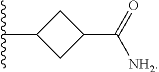 [SMILES: *C1CC(C(N)=O)C1]

In embodiments, R⁹ is

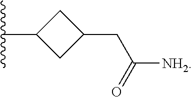 [SMILES: *C1CC(CC(N)=O)C1]

In embodiments, R⁹ is

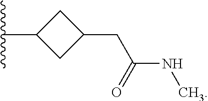 [SMILES: *C1CC(CC(=O)NC)C1]

In embodiments, R⁹ is

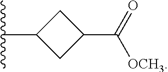 [SMILES: *C1CC(C(=O)OC)C1]

In embodiments, R⁹ is

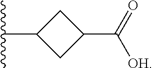 [SMILES: *C1CC(C(=O)O)C1]

In embodiments, R⁹ is

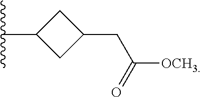 [SMILES: *C1CC(CC(=O)OC)C1]

In embodiments, R⁹ is

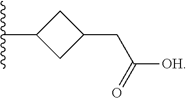 [SMILES: *C1CC(CC(=O)O)C1]

In embodiments, R⁹ is

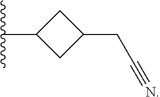 [SMILES: *C1CC(CC#N)C1]

In embodiments, R⁹ is

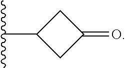 [SMILES: *C1CC(=O)C1]

In embodiments, R⁹ is

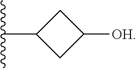 [SMILES: *C1CC(O)C1]

In embodiments, R⁹ is

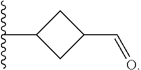 [SMILES: *C1CC(C=O)C1]

In embodiments, R⁹ is

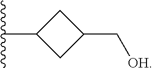 [SMILES: *C1CC(CO)C1]

In embodiments, R⁹ is

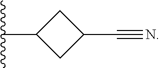 [SMILES: *C1CC(C#N)C1]

In embodiments, R⁹ is

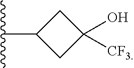 [SMILES: *C1CC(O)(C(F)(F)F)C1]

In embodiments, R⁹ is

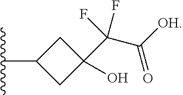 [SMILES: *C1CC(O)(C(F)(F)C(=O)O)C1]

In embodiments, R⁹ is

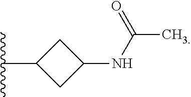 [SMILES: *C1CC(NC(C)=O)C1]

In embodiments, R⁹ is

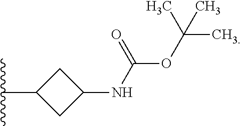 [SMILES: *C1CC(NC(=O)OC(C)(C)C)C1]

In embodiments, R⁹ is

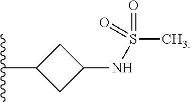 [SMILES: *C1CC(NS(C)(=O)=O)C1]

In embodiments, R⁹ is

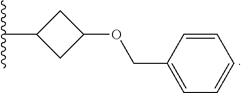 [SMILES: *C1CC(OCc2ccccc2)C1]

In embodiments, R⁹ is

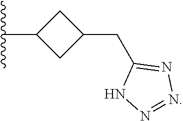 [SMILES: *C1CC(Cc2nnn[nH]2)C1]

In embodiments, R⁹ is

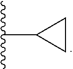 [SMILES: *C1CC1 ; "."]

In embodiments, R⁹ is

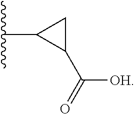 [SMILES: *C1CC1C(=O)O]

In embodiments, R⁹ is

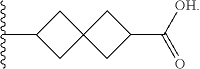 [SMILES: *C1CC2(C1)CC(C(=O)O)C2]

In embodiments, R⁹ is

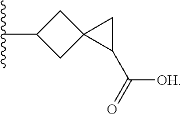 [SMILES: *C1CC2(C1)CC2C(=O)O]

In embodiments, R⁹ is

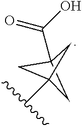 [SMILES: *C12CC(C(=O)O)(C1)C2 ; "."]

In embodiments, R⁹ is

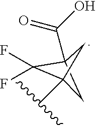 [SMILES: *C12CC(C(=O)O)(C1)C2(F)F ; "."]

In embodiments, R⁹ is

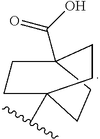 [SMILES: *C12CCC(C(=O)O)(CC1)CC2 ; "."]

In embodiments, R⁹ is

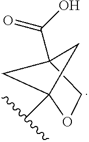 [SMILES: *C12CC(C(=O)O)(CO1)C2 ; "."]

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D.1}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B.1}, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5D}, R^{5D.1}, R^{5D.2}, and R^{5D.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R^{6A} is substituted, R^{6A} is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A}, R^{6A.1}, R^{6A.2}, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6A}, R^{6A.1}, R^{6A.2}, and R^{6A.3}, respectively.

In embodiments, when R^{6B} is substituted, R^{6B} is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}, R^{6B.1}, R^{6B.2}, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6B}, R^{6B.1}, R^{6B.2}, and R^{6B.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A.1}, R^{6A.2}, and R^{6A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6A.1}, R^{6A.2}, and R^{6A.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B.1}, R^{6B.2}, and R^{6B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6B.1}, R^{6B.2}, and R^{6B.3}, respectively.

In embodiments, when R^{6C} is substituted, R^{6C} is substituted with one or more first substituent groups denoted by R^{6C.1} as explained in the definitions section above in the description of “first substituent group(s)”. In embodiments, when an R^{6C.1} substituent group is substituted, the R^{6C.1} substituent group is substituted with one or more second substituent groups denoted by R^{6C.2} as explained in the definitions section above in the description of “first substituent group(s)”. In embodiments, when an R^{6C.2} substituent group is substituted, the R^{6C.2} substituent group is substituted with one or more third substituent groups denoted by R^{6C.3} as explained in the definitions section above in the description of “first substituent group(s)”. In the above embodiments, R^{6C}, R^{6C.1}, R^{6C.2}, and R^{6C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6C}, R^{6C.1}, R^{6C.2}, and R^{6C.3}, respectively.

In embodiments, when R^{6D} is substituted, R^{6D} is substituted with one or more first substituent groups denoted by R^{6D.1} as explained in the definitions section above in the description of “first substituent group(s)”. In embodiments, when an R^{6D.1} substituent group is substituted, the R^{6D.1} substituent group is substituted with one or more second substituent groups denoted by R^{6D.2} as explained in the definitions section above in the description of “first substituent group(s)”. In embodiments, when an R^{6D.2} substituent group is substituted, the R^{6D.2} substituent group is substituted with one or more third substituent groups denoted by R^{6D.3} as explained in the definitions section above in the description of “first substituent group(s)”. In the above embodiments, R^{6D}, R^{6D.1}, R^{6D.2}, and R^{6D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6D}, R^{6D.1}, R^{6D.2}, and R^{6D.3}, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R^{7.3}, respectively.

In embodiments, when R^{7A} is substituted, R^{7A} is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A}, R^{7A.1}, R^{7A.2}, and R^{7A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7A}, R^{7A.1}, R^{7A.2}, and R^{7A.3}, respectively.

In embodiments, when R^{7B} is substituted, R^{7B} is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B}, R^{7B.1}, R^{7B.2}, and R^{7B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7B}, R^{7B.1}, R^{7B.2}, and R^{7B.3}, respectively.

In embodiments, when R^{7A} and R^{7B} substituents bonded to the same nitrogen atom are optionally joined to form a moiety that is substituted (e.g., a substituted heterocycloalkyl or substituted heteroaryl), the moiety is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A.1}, R^{7A.2}, and R^{7A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7A.1}, R^{7A.2}, and R^{7A.3}, respectively.

In embodiments, when R^{7A} and R^{7B} substituents bonded to the same nitrogen atom are optionally joined to form a moiety that is substituted (e.g., a substituted heterocycloalkyl or substituted heteroaryl), the moiety is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B.1}, R^{7B.2}, and R^{7B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7B.1}, R^{7B.2}, and R^{7B.3}, respectively.

In embodiments, when R^{7C} is substituted, R^{7C} is substituted with one or more first substituent groups denoted by R^{7C.1} as explained in the definitions section above in the description of “first substituent group(s)”. In embodiments, when an R^{7C.1} substituent group is substituted, the R^{7C.1} substituent group is substituted with one or more second substituent groups denoted by R^{7C.2} as explained in the definitions section above in the description of “first substituent group(s)”. In embodiments, when an R^{7C.2} substituent group is substituted, the R^{7C.2} substituent group is substituted with one or more third substituent groups denoted by R^{7C.3} as explained in the definitions section above in the description of “first substituent group(s)”. In the above embodiments, R^{7C}, R^{7C.1}, R^{7C.2}, and R^{7C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7C}, R^{7C.1}, R^{7C.2}, and R^{7C.3}, respectively.

In embodiments, when R^{7D} is substituted, R^{7D} is substituted with one or more first substituent groups denoted by R^{7D.1} as explained in the definitions section above in the description of “first substituent group(s)”. In embodiments, when an R^{7D.1} substituent group is substituted, the R^{7D.1} substituent group is substituted with one or more second substituent groups denoted by R^{7D.2} as explained in the definitions section above in the description of “first substituent group(s)”. In embodiments, when an R^{7D.2} substituent group is substituted, the R^{7D.2} substituent group is substituted with one or more third substituent groups denoted by R^{7D.3} as explained in the definitions section above in the description of “first substituent group(s)”. In the above embodiments, R^{7D}, R^{7D.1}, R^{7D.2}, and R^{7D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7D}, R^{7D.1}, R^{7D.2}, and R^{7D.3}, respectively.

In embodiments, when R⁸ is substituted, R⁸ is substituted with one or more first substituent groups denoted by R^{8.1} as explained in the definitions section above in the description of “first substituent group(s)”. In embodiments, when an R^{8.1} substituent group is substituted, the R^{8.1} substituent group is substituted with one or more second substituent groups denoted by R^{8.2} as explained in the definitions section above in the description of “first substituent group(s)”. In embodiments, when an R^{8.2} substituent group is substituted, the R^{8.2} substituent group is substituted with one or more third substituent groups denoted by R^{8.3} as explained in the definitions section above in the description of “first substituent group(s)”. In the above embodiments, R⁸, R^{8.1}, R^{8.2}, and R^{8.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸, R¹, R^{8.2}, and R^{8.3}, respectively.

In embodiments, when R^{8A} is substituted, R^{8A} is substituted with one or more first substituent groups denoted by R^{8A.1} as explained in the definitions section above in the description of “first substituent group(s)”. In embodiments, when an R^{8A.1} substituent group is substituted, the R^{8A.1} substituent group is substituted with one or more second substituent groups denoted by R^{8A.2} as explained in the definitions section above in the description of “first substituent group(s)”. In embodiments, when an R^{8A.2} substituent group is substituted, the R^{8A.2} substituent group is substituted with one or more third substituent groups denoted by R^{8A.3} as explained in the definitions section above in the description of “first substituent group(s)”. In the above embodiments, R^{8A}, R^{8A.1}, R^{8A.2}, and R^{8A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8A}, R^{8A.1}, R^{8A.2}, and R^{8A.3}, respectively.

In embodiments, when R^{8B} is substituted, R^{8B} is substituted with one or more first substituent groups denoted by R^{8B.1} as explained in the definitions section above in the description of “first substituent group(s)”. In embodiments, when an R^{8B.1} substituent group is substituted, the R^{8B.1} substituent group is substituted with one or more second substituent groups denoted by R^{8B.2} as explained in the definitions section above in the description of “first substituent group(s)”. In embodiments, when an R^{8B.2} substituent group is substituted, the R^{8B.2} substituent group is substituted with one or more third substituent groups denoted by R^{8B.3} as explained in the definitions section above in the description of “first substituent group(s)”. In the above embodiments, R^{8B}, R^{8B.1}, R^{8B.2}, and R^{8B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8B}, R^{8B.1}, R^{8B.2}, and R^{8B.3}, respectively.

In embodiments, when R^{8A} and R^{8B} substituents bonded to the same nitrogen atom are optionally joined to form a moiety that is substituted (e.g., a substituted heterocycloalkyl or substituted heteroaryl), the moiety is substituted with one or more first substituent groups denoted by R^{8A.1} as explained in the definitions section above in the description of “first substituent group(s)”. In embodiments, when an R^{8A.1} substituent group is substituted, the R^{8A.1} substituent group is substituted with one or more second substituent groups denoted by R^{8A.2} as explained in the definitions section above in the description of “first substituent group(s)”. In embodiments, when an R^{8A.2} substituent group is substituted, the R^{8A.2} substituent group is substituted with one or more third substituent groups denoted by R^{8A.3} as explained in the definitions section above in the description of “first substituent group(s)”. In the above embodiments, R^{8A.1}, R^{8A.2}, and R^{8A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8A.1}, R^{8A.2}, and R^{8A.3}, respectively.

In embodiments, when R^{8A} and R^{8B} substituents bonded to the same nitrogen atom are optionally joined to form a moiety that is substituted (e.g., a substituted heterocycloalkyl or substituted heteroaryl), the moiety is substituted with one or more first substituent groups denoted by R^{8B.1} as explained in the definitions section above in the description of “first substituent group(s)”. In embodiments, when an R^{8B.1} substituent group is substituted, the R^{8B.1} substituent group is substituted with one or more second substituent groups denoted by R^{8B.2} as explained in the definitions section above in the description of “first substituent group(s)”. In embodiments, when an R^{8B.2} substituent group is substituted, the R^{8B.2} substituent group is substituted with one or more third substituent groups denoted by R^{8B.3} as explained in the definitions section above in the description of “first substituent group(s)”. In the above embodiments, R^{8B.1}, R^{8B.2}, and R^{8B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8B.1}, R^{8B.2}, and R^{8B.3}, respectively.

In embodiments, when R^{8C} is substituted, R^{8C} is substituted with one or more first substituent groups denoted by R^{8C.1} as explained in the definitions section above in the description of “first substituent group(s)”. In embodiments, when an R^{8C.1} substituent group is substituted, the R^{8C.1} substituent group is substituted with one or more second substituent groups denoted by R^{8C.2} as explained in the definitions section above in the description of “first substituent group(s)”. In embodiments, when an R^{8C.2} substituent group is substituted, the R^{8C.2} substituent group is substituted with one or more third substituent groups denoted by R^{8C.3} as explained in the definitions section above in the description of “first substituent group(s)”. In the above embodiments, R^{8C}, R^{8C.1}, R^{8C.2}, and R^{8C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8C}, R^{8C.1}, R^{8C.2}, and R^{8C.3}, respectively.

In embodiments, when R^{8D} is substituted, R^{8D} is substituted with one or more first substituent groups denoted by R^{8D.1} as explained in the definitions section above in the description of “first substituent group(s)”. In embodiments, when an R^{8D.1} substituent group is substituted, the R^{8D.1} substituent group is substituted with one or more second substituent groups denoted by R^{8D.2} as explained in the definitions section above in the description of “first substituent group(s)”. In embodiments, when an R^{8D.2} substituent group is substituted, the R^{8D.2} substituent group is substituted with one or more third substituent groups denoted by R^{8D.3} as explained in the definitions section above in the description of “first substituent group(s)”. In the above embodiments, R^{8D}, R^{8D.1}, R^{8D.2}, and R^{8D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8D}, R^{8D.1}, R^{8D.2}, and R^{8D.3}, respectively.

In embodiments, when R^{8.1} is substituted, R^{8.1} is substituted with one or more first substituent groups denoted by R^{8.1.1} as explained in the definitions section above in the description of “first substituent group(s)”. In embodiments, when an R^{8.1.1} substituent group is substituted, the R^{8.1.1} substituent group is substituted with one or more second substituent groups denoted by R^{8.1.2} as explained in the definitions section above in the description of “first substituent group(s)”. In embodiments, when an R^{8.1.2} substituent group is substituted, the R^{8.1.2} substituent group is substituted with one or more third substituent groups denoted by R^{8.1.3} as explained in the definitions section above in the description of “first substituent group(s)”. In the above embodiments, R^{8.1}, R^{8.1.1}, R^{8.1.2}, and R^{8.1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{8.1.1}, R^{8.1.2}, and R^{8.1.3}, respectively.

In embodiments, when R^{8.2} is substituted, R^{8.2} is substituted with one or more first substituent groups denoted by R^{8.2.1} as explained in the definitions section above in the description of “first substituent group(s)”. In embodiments, when an R^{8.2.1} substituent group is substituted, the R^{8.2.1} substituent group is substituted with one or more second substituent groups denoted by R^{8.2.2} as explained in the definitions section above in the description of “first substituent group(s)”. In embodiments, when an R^{8.2.2} substituent group is substituted, the R^{8.2.2} substituent group is substituted with one or more third substituent groups denoted by R^{8.2.3} as explained in the definitions section above in the description of “first substituent group(s)”. In the above embodiments, R^{8.2}, R^{8.2.1}, R^{8.2.2}, and R^{8.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8.2}, R^{8.2.1}, R^{8.2.2}, and R^{8.2.3}, respectively.

In embodiments, when R^{8.3} is substituted, R^{8.3} is substituted with one or more first substituent groups denoted by R^{8.3.1} as explained in the definitions section above in the description of “first substituent group(s)”. In embodiments, when an R^{8.3.1} substituent group is substituted, the R^{8.3.1} substituent group is substituted with one or more second substituent groups denoted by R^{8.3.2} as explained in the definitions section above in the description of “first substituent group(s)”. In embodiments, when an R^{8.3.2} substituent group is substituted, the R^{8.3.2} substituent group is substituted with one or more third substituent groups denoted by R^{8.3.3} as explained in the definitions section above in the description of “first substituent group(s)”. In the above embodiments, R^{8.3}, R^{8.3.1}, R^{8.3.2}, and R^{8.3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8.3}, R^{8.3.1}, R^{8.3.2}, and R^{8.3.3}, respectively.

In embodiments, when R^{8.2} and R^{8.3} substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{8.2.1} as explained in the definitions section above in the description of “first substituent group(s)”. In embodiments, when an R^{8.2.1} substituent group is substituted, the R^{8.2.1} substituent group is substituted with one or more second substituent groups denoted by R^{8.2.2} as explained in the definitions section above in the description of “first substituent group(s)”. In embodiments, when an R^{8.2.2} substituent group is substituted, the R^{8.2.2} substituent group is substituted with one or more third substituent groups denoted by R^{2.3} as explained in the definitions section above in the description of “first substituent group(s)”. In the above embodiments, R^{8.2.1}, R^{8.2.2}, and R^{8.2.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8.2.1}, R^{8.2.2}, and R^{8.2.3}, respectively.

In embodiments, when R^{8.2} and R^{8.3} substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{8.3.1} as explained in the definitions section above in the description of “first substituent group(s)”. In embodiments, when an R^{8.3.1} substituent group is substituted, the R^{8.3.1} substituent group is substituted with one or more second substituent groups denoted by R^{8.3.2} as explained in the definitions section above in the description of “first substituent group(s)”. In embodiments, when an R^{8.3.2} substituent group is substituted, the R^{8.3.2} substituent group is substituted with one or more third substituent groups denoted by R^{8.3.3} as explained in the definitions section above in the description of “first substituent group(s)”. In the above embodiments, R^{8.3.1}, R^{8.3.2}, and R^{8.3.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8.3.1}, R^{8.3.2}, and R^{8.3.3}, respectively.

In embodiments, when R⁹ is substituted, R⁹ is substituted with one or more first substituent groups denoted by R^{9.1} as explained in the definitions section above in the description of “first substituent group(s)”. In embodiments, when an R^{9.1} substituent group is substituted, the R^{9.1} substituent group is substituted with one or more second substituent groups denoted by R^{9.2} as explained in the definitions section above in the description of “first substituent group(s)”. In embodiments, when an R^{9.2} substituent group is substituted, the R^{9.2} substituent group is substituted with one or more third substituent groups denoted by R^{9.3} as explained in the definitions section above in the description of “first substituent group(s)”. In the above embodiments, R⁹, R^{9.1}, R^{9.2}, and R^{9.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁹, R^{9.1}, R^{9.2}, and R^{9.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰, R^{10.1}, R^{10.2}, and R^{10.3}, respectively.

In embodiments, when R^{10A} is substituted, R^{10A} is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10A}, R^{10A.1}, R^{10A.2} and R^{10A.3} respectively.

In embodiments, when R^{10B} is substituted, R^{10B} is substituted with one or more first substituent groups denoted by R^{10B.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10B}, R^{10B.1}, R^{10B.2}, and R^{10B.3} respectively.

In embodiments, when R^{10A} and R^{10B} substituents bonded to the same nitrogen atom are optionally joined to form a moiety that is substituted (e.g., a substituted heterocycloalkyl or substituted heteroaryl), the moiety is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A.1}, R^{10A.2}, and R^{10A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10A.1}, R^{10A.2}, and R^{10A.3}, respectively.

In embodiments, when R^{10A} and R^{10B} substituents bonded to the same nitrogen atom are optionally joined to form a moiety that is substituted (e.g., a substituted heterocycloalkyl or substituted heteroaryl), the moiety is substituted with one or more first substituent groups denoted by R^{10B.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B.1}, R^{10B.2}, and R^{10B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10B.1}, R^{10B.2}, and R^{10B.3}, respectively.

In embodiments, when R^{10C} is substituted, R^{10C} is substituted with one or more first substituent groups denoted by R^{10C.1} as explained in the definitions section above in the description of “first substituent group(s)”. In embodiments, when an R^{10C.1} substituent group is substituted, the R^{10C.1} substituent group is substituted with one or more second substituent groups denoted by R^{10C.2} as explained in the definitions section above in the description of “first substituent group(s)”. In embodiments, when an R^{10C.2} substituent group is substituted, the R^{10C.2} substituent group is substituted with one or more third substituent groups denoted by R^{10C.3} as explained in the definitions section above in the description of “first substituent group(s)”. In the above embodiments, R^{10C}, R^{10C.1}, R^{10C.2}, and R^{10C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10C}, R^{10C.1}, R^{10C.2}, and R^{10C.3} respectively.

In embodiments, when R^{10D} is substituted, R^{10D} is substituted with one or more first substituent groups denoted by R^{10D} as explained in the definitions section above in the description of “first substituent group(s)”. In embodiments, when an R^{10D.1} substituent group is substituted, the R^{10D.1} substituent group is substituted with one or more second substituent groups denoted by R^{10D.2} as explained in the definitions section above in the description of “first substituent group(s)”. In embodiments, when an R^{10D.2} substituent group is substituted, the R^{10D.2} substituent group is substituted with one or more third substituent groups denoted by R^{10D.3} as explained in the definitions section above in the description of “first substituent group(s)”. In the above embodiments, R^{10D}, R^{10D.1}, R^{10D.2}, and R^{10D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10D}, R^{10D.1}, R^{10D.2}, and R^{10D.3} respectively.

In embodiments, when R¹¹ is substituted, R¹¹ is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R¹¹, R^{11.1}, R^{11.2}, and R^{11.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹¹, R^{11.1}, R^{11.2}, and R^{11.3}, respectively.

In embodiments, when R^{11A} is substituted, R^{11A} is substituted with one or more first substituent groups denoted by R^{11A.1} as explained in the definitions section above in the description of “first substituent group(s)”. In embodiments, when an R^{11A.1} substituent group is substituted, the R^{11A.1} substituent group is substituted with one or more second substituent groups denoted by R^{11A.2} as explained in the definitions section above in the description of “first substituent group(s)”. In embodiments, when an R^{11A.2} substituent group is substituted, the R^{11A.2} substituent group is substituted with one or more third substituent groups denoted by R^{11A.3} as explained in the definitions section above in the description of “first substituent group(s)”. In the above embodiments, R^{11A}, R^{11A.1}, R^{11A.2}, and R^{11A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11A}, R^{11A.1}, R^{11A.2} and R^{11A.3} respectively.

In embodiments, when R^{11B} is substituted, R^{11B} is substituted with one or more first substituent groups denoted by R^{11B.1} as explained in the definitions section above in the description of “first substituent group(s)”. In embodiments, when an R^{11B.1} substituent group is substituted, the R^{11B.1} substituent group is substituted with one or more second substituent groups denoted by R^{11B.2} as explained in the definitions section above in the description of “first substituent group(s)”. In embodiments, when an R^{11B.2} substituent group is substituted, the R^{11B.2} substituent group is substituted with one or more third substituent groups denoted by R^{11B.3} as explained in the definitions section above in the description of “first substituent group(s)”. In the above embodiments, R^{11B}, R^{11B.1}, R^{11B.2}, and R^{11B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11B}, R^{11B.1}, R^{11B.2}, and R^{11B.3} respectively.

In embodiments, when R^{11A} and R^{11B} substituents bonded to the same nitrogen atom are optionally joined to form a moiety that is substituted (e.g., a substituted heterocycloalkyl or substituted heteroaryl), the moiety is substituted with one or more first substituent groups denoted by R^{11A.1} as explained in the definitions section above in the description of “first substituent group(s)”. In embodiments, when an R^{11A.1} substituent group is substituted, the R^{11A.1} substituent group is substituted with one or more second substituent groups denoted by R^{11A.2} as explained in the definitions section above in the description of “first substituent group(s)”. In embodiments, when an R^{11A.2} substituent group is substituted, the R^{11A.2} substituent group is substituted with one or more third substituent groups denoted by R^{11A.3} as explained in the definitions section above in the description of “first substituent group(s)”. In the above embodiments, R^{11A.1}, R^{11A.2}, and R^{11A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{11A.1}, R^{11A.2}, and R^{11A.3}, respectively.

In embodiments, when R^{11A} and R^{11B} substituents bonded to the same nitrogen atom are optionally joined to form a moiety that is substituted (e.g., a substituted heterocycloalkyl or substituted heteroaryl), the moiety is substituted with one or more first substituent groups denoted by R^{11B.1} as explained in the definitions section above in the description of “first substituent group(s)”. In embodiments, when an R^{11B.1} substituent group is substituted, the R^{11B.1} substituent group is substituted with one or more second substituent groups denoted by R^{11B.2} as explained in the definitions section above in the description of “first substituent group(s)”. In embodiments, when an R^{11B.2} substituent group is substituted, the R^{11B.2} substituent group is substituted with one or more third substituent groups denoted by R^{11B.3} as explained in the definitions section above in the description of “first substituent group(s)”. In the above embodiments, R^{11B.1}, R^{11B.2}, and R^{11B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{11B.1}, R^{11B.2}, and R^{11B.3}, respectively.

In embodiments, when R^{11C} is substituted, R^{11C} is substituted with one or more first substituent groups denoted by R^{11C.1} as explained in the definitions section above in the description of “first substituent group(s)”. In embodiments, when an R^{11C.1} substituent group is substituted, the R^{11C.1} substituent group is substituted with one or more second substituent groups denoted by R^{11C.2} as explained in the definitions section above in the description of “first substituent group(s)”. In embodiments, when an R^{11C.2} substituent group is substituted, the R^{11C.2} substituent group is substituted with one or more third substituent groups denoted by R^{11C.3} as explained in the definitions section above in the description of “first substituent group(s)”. In the above embodiments, R^{11C}, R^{11C.1}, R^{11C.2}, and R^{11C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11C}, R^{11C.1}, R^{11C.2} and R^{11C.3} respectively.

In embodiments, when R^{11D} is substituted, R^{11D} is substituted with one or more first substituent groups denoted by R^{11D.1} as explained in the definitions section above in the description of “first substituent group(s)”. In embodiments, when an R^{11D.1} substituent group is substituted, the R^{11D.1} substituent group is substituted with one or more second substituent groups denoted by R^{11D.2} as explained in the definitions section above in the description of “first substituent group(s)”. In embodiments, when an R^{11D.2} substituent group is substituted, the R^{11D.2} substituent group is substituted with one or more third substituent groups denoted by R^{11D.3} as explained in the definitions section above in the description of “first substituent group(s)”. In the above embodiments, R^{11D}, R^{11D.1}, R^{11D.2}, and R^{11D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11D}, R^{11D.1}, R^{11D.2}, and R^{11D.3} respectively.

In embodiments, when R¹² is substituted, R¹² is substituted with one or more first substituent groups denoted by R^{12.1} as explained in the definitions section above in the description of “first substituent group(s)”. In embodiments, when an R^{12.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{12.2} substituent group is substituted with one or more third substituent groups denoted by R^{12.3} as explained in the definitions section above in the description of “first substituent group(s)”. In the above embodiments, R¹², R^{12.1}, R^{12.2}, and R^{12.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R¹², R^{12.1}, R^{12.2}, and R^{12.3}, respectively.

In embodiments, when R^{12A} is substituted, R^{12A} is substituted with one or more first substituent groups denoted by R^{12A.1} as explained in the definitions section above in the description of “first substituent group(s)”. In embodiments, when an R^{12A.1} substituent group is substituted, the R^{12A.1} substituent group is substituted with one or more second substituent groups denoted by R^{12A.2} as explained in the definitions section above in the description of “first substituent group(s)”. In embodiments, when an R^{12A.2} substituent group is substituted, the R^{12A.2} substituent group is substituted with one or more third substituent groups denoted by R^{12A.3} as explained in the definitions section above in the description of “first substituent group(s)”. In the above embodiments, R^{12A}, R^{12A.1}, R^{12A.2}, and R^{12A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12A}, R^{12A.1}, R^{12A.2}, and R^{12A.3} respectively.

In embodiments, when R^{12B} is substituted, R^{12B} is substituted with one or more first substituent groups denoted by R^{12B.1} as explained in the definitions section above in the description of “first substituent group(s)”. In embodiments, when an R^{12B.1} substituent group is substituted, the R^{12B.1} substituent group is substituted with one or more second substituent groups denoted by R^{12B.2} as explained in the definitions section above in the description of “first substituent group(s)”. In embodiments, when an R^{12B.2} substituent group is substituted, the R^{12B.2} substituent group is substituted with one or more third substituent groups denoted by R^{12B.3} as explained in the definitions section above in the description of “first substituent group(s)”. In the above embodiments, R^{12B}, R^{12B.1}, R^{12B.2}, and R^{12B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12B}, R^{12B.1}, R^{12B.2}, and R^{12B.3}, respectively.

In embodiments, when R^{12A} and R^{12B} substituents bonded to the same nitrogen atom are optionally joined to form a moiety that is substituted (e.g., a substituted heterocycloalkyl or substituted heteroaryl), the moiety is substituted with one or more first substituent groups denoted by R^{12A.1} as explained in the definitions section above in the description of “first substituent group(s)”. In embodiments, when an R^{12A.1} substituent group is substituted, the R^{12A.1} substituent group is substituted with one or more second substituent groups denoted by R^{12A.2} as explained in the definitions section above in the description of “first substituent group(s)”. In embodiments, when an R^{12A.2} substituent group is substituted, the R^{12A.2} substituent group is substituted with one or more third substituent groups denoted by R^{12A.3} as explained in the definitions section above in the description of “first substituent group(s)”. In the above embodiments, R^{12A.1}, R^{12A.2}, and R^{12A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{12A.1}, R^{12A.2}, and R^{12A.3}, respectively.

In embodiments, when R^{12A} and R^{12B} substituents bonded to the same nitrogen atom are optionally joined to form a moiety that is substituted (e.g., a substituted heterocycloalkyl or substituted heteroaryl), the moiety is substituted with one or more first substituent groups denoted by R^{12B.1} as explained in the definitions section above in the description of “first substituent group(s)”. In embodiments, when an R^{12B.1} substituent group is substituted, the R^{12B.1} substituent group is substituted with one or more second substituent groups denoted by R^{12B.2} as explained in the definitions section above in the description of “first substituent group(s)”. In embodiments, when an R^{12B.2} substituent group is substituted, the R^{12B.2} substituent group is substituted with one or more third substituent groups denoted by R^{12B.3} as explained in the definitions section above in the description of “first substituent group(s)”. In the above embodiments, R^{12B.1}, R^{12B.2}, and R^{12B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{12B.1}, R^{12B.2}, and R^{12B.3}, respectively.

In embodiments, when R^{12C} is substituted, R^{12C} is substituted with one or more first substituent groups denoted by R^{12C.1} as explained in the definitions section above in the description of “first substituent group(s)”. In embodiments, when an R^{12C.1} substituent group is substituted, the R^{12C.1} substituent group is substituted with one or more second substituent groups denoted by R^{12C.2} as explained in the definitions section above in the description of “first substituent group(s)”. In embodiments, when an R^{12C.2} substituent group is substituted, the R^{12C.2} substituent group is substituted with one or more third substituent groups denoted by R^{12C.3} as explained in the definitions section above in the description of “first substituent group(s)”. In the above embodiments, R^{12C}, R^{12C.1}, R^{12C.2}, and R^{12C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12C}, R^{12C.1}, R^{12C.2}, and R^{12C.3} respectively.

In embodiments, when R^{12D} is substituted, R^{12D} is substituted with one or more first substituent groups denoted by R^{12D.1} as explained in the definitions section above in the description of “first substituent group(s)”. In embodiments, when an R^{12D.1} substituent group is substituted, the R^{12D.1} substituent group is substituted with one or more second substituent groups denoted by R^{12D.2} as explained in the definitions section above in the description of “first substituent group(s)”. In embodiments, when an R^{12D.2} substituent group is substituted, the R^{12D.2} substituent group is substituted with one or more third substituent groups denoted by R^{12D.3} as explained in the definitions section above in the description of “first substituent group(s)”. In the above embodiments, R^{12D}, R^{12D.1}, R^{12D.2}, and R^{12D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12D}, R^{12D.1}, R^{12D.2} and R^{12D.3} respectively.

In embodiments, when R² and R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R^{2.1}, R^{2.2}, and R^{2.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2.1}, R^{2.2}, and R^{2.3}, respectively.

In embodiments, when R² and R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R^{3.1}, R^{3.2}, and R^{3.3} have values corresponding to the values of R^{WW.1}, R^{WW.2} and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R³ and R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R^{3.1}, R^{3.2}, and R^{3.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R³ and R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R^{4.1}, R^{4.2}, and R^{4.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4.1}, R^{4.2}, and R^{4.3}, respectively.

In embodiments, when R⁴ and R⁵ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R^{4.1}, R^{4.2}, and R^{4.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4.1}, R^{4.2}, and R^{4.3}, respectively.

In embodiments, when R⁴ and R⁵ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R^{5.1}, R^{5.2}, and R^{5.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5.1}, R^{5.2}, and R^{5.3}, respectively.

In embodiments, when R¹⁰ and R² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R^{10.1}, R^{10.2}, and R^{10.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10.1}, R^{10.2}, and R^{10.3}, respectively.

In embodiments, when R¹⁰ and R² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R^{2.1}, R^{2.2}, and R^{2.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2.1}, R^{2.2}, and R^{2.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, the compound is

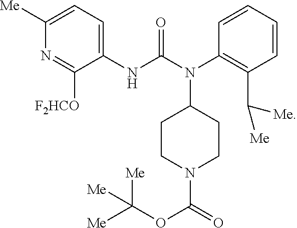 [SMILES: Cc1ccc(NC(=O)N(c2ccccc2C(C)C)C2CCN(C(=O)OC(C)(C)C)CC2)c(OC(F)F)n1]

In embodiments, the compound is

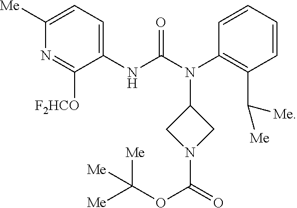 [SMILES: Cc1ccc(NC(=O)N(c2ccccc2C(C)C)C2CN(C(=O)OC(C)(C)C)C2)c(OC(F)F)n1]

In embodiments, the compound is

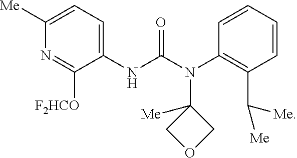 [SMILES: Cc1ccc(NC(=O)N(c2ccccc2C(C)C)C2(C)COC2)c(OC(F)F)n1]

In embodiments, the compound is

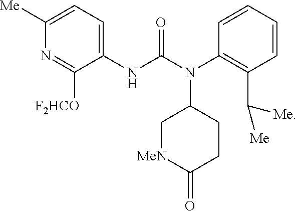 [SMILES: Cc1ccc(NC(=O)N(c2ccccc2C(C)C)C2CCC(=O)N(C)C2)c(OC(F)F)n1]

In embodiments, the compound is

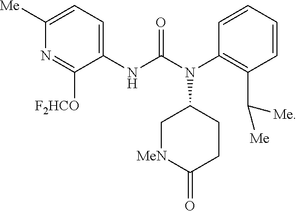 [SMILES: Cc1ccc(NC(=O)N(c2ccccc2C(C)C)[C@@H]2CCC(=O)N(C)C2)c(OC(F)F)n1]

In embodiments, the compound is

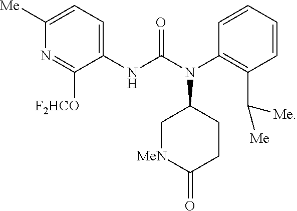 [SMILES: Cc1ccc(NC(=O)N(c2ccccc2C(C)C)[C@H]2CCC(=O)N(C)C2)c(OC(F)F)n1]

In embodiments, the compound is

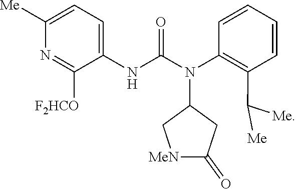 [SMILES: Cc1ccc(NC(=O)N(c2ccccc2C(C)C)C2CC(=O)N(C)C2)c(OC(F)F)n1]

In embodiments, the compound is

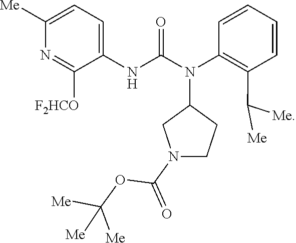 [SMILES: Cc1ccc(NC(=O)N(c2ccccc2C(C)C)C2CCN(C(=O)OC(C)(C)C)C2)c(OC(F)F)n1]

In embodiments, the compound is

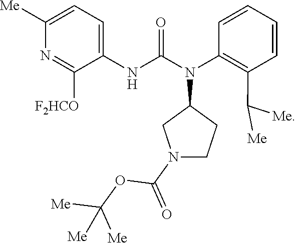 [SMILES: Cc1ccc(NC(=O)N(c2ccccc2C(C)C)[C@H]2CCN(C(=O)OC(C)(C)C)C2)c(OC(F)F)n1]

In embodiments, the compound is

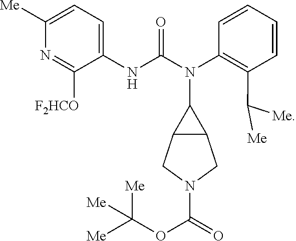 [SMILES: Cc1ccc(NC(=O)N(c2ccccc2C(C)C)C2C3CN(C(=O)OC(C)(C)C)CC32)c(OC(F)F)n1]

In embodiments, the compound is

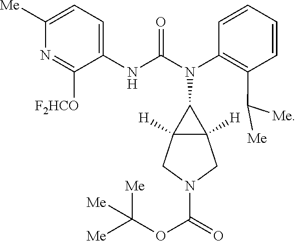 [SMILES: Cc1ccc(NC(=O)N(c2ccccc2C(C)C)[C@H]2[C@@H]3CN(C(=O)OC(C)(C)C)C[C@@H]32)c(OC(F)F)n1]

In embodiments, the compound is

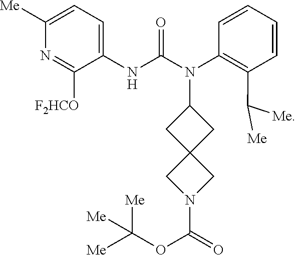 [SMILES: Cc1ccc(NC(=O)N(c2ccccc2C(C)C)C2CC3(C2)CN(C(=O)OC(C)(C)C)C3)c(OC(F)F)n1]

In embodiments, the compound is

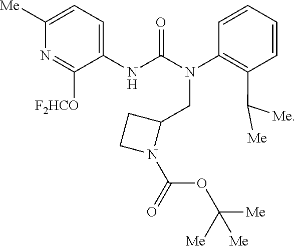 [SMILES: Cc1ccc(NC(=O)N(CC2CCN2C(=O)OC(C)(C)C)c2ccccc2C(C)C)c(OC(F)F)n1]

In embodiments, the compound is

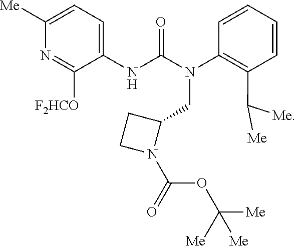 [SMILES: Cc1ccc(NC(=O)N(C[C@H]2CCN2C(=O)OC(C)(C)C)c2ccccc2C(C)C)c(OC(F)F)n1]

In embodiments, the compound is

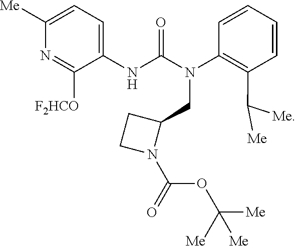 [SMILES: Cc1ccc(NC(=O)N(C[C@@H]2CCN2C(=O)OC(C)(C)C)c2ccccc2C(C)C)c(OC(F)F)n1]

In embodiments, the compound is

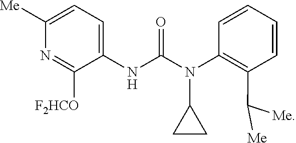 [SMILES: Cc1ccc(NC(=O)N(c2ccccc2C(C)C)C2CC2)c(OC(F)F)n1]

In embodiments, the compound is

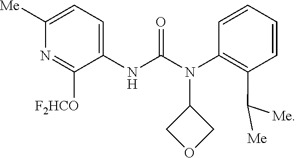 [SMILES: Cc1ccc(NC(=O)N(c2ccccc2C(C)C)C2COC2)c(OC(F)F)n1]

In embodiments, the compound is

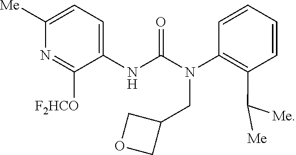 [SMILES: Cc1ccc(NC(=O)N(CC2COC2)c2ccccc2C(C)C)c(OC(F)F)n1]

In embodiments, the compound is

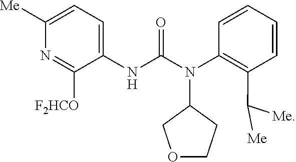 [SMILES: Cc1ccc(NC(=O)N(c2ccccc2C(C)C)C2CCOC2)c(OC(F)F)n1]

In embodiments, the compound is

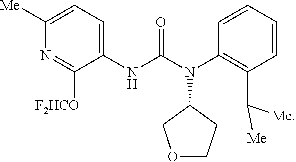 [SMILES: Cc1ccc(NC(=O)N(c2ccccc2C(C)C)[C@@H]2CCOC2)c(OC(F)F)n1]

In embodiments, the compound is

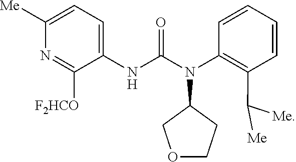 [SMILES: Cc1ccc(NC(=O)N(c2ccccc2C(C)C)[C@H]2CCOC2)c(OC(F)F)n1]

In embodiments, the compound is

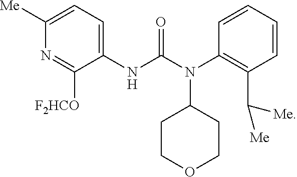 [SMILES: Cc1ccc(NC(=O)N(c2ccccc2C(C)C)C2CCOCC2)c(OC(F)F)n1]

In embodiments, the compound is

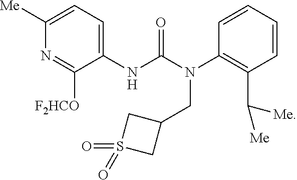 [SMILES: Cc1ccc(NC(=O)N(CC2CS(=O)(=O)C2)c2ccccc2C(C)C)c(OC(F)F)n1]

In embodiments, the compound is

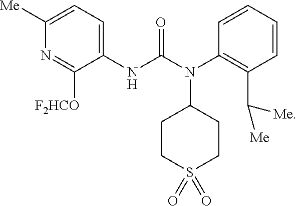 [SMILES: Cc1ccc(NC(=O)N(c2ccccc2C(C)C)C2CCS(=O)(=O)CC2)c(OC(F)F)n1]

In embodiments, the compound is

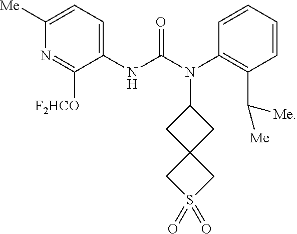 [SMILES: Cc1ccc(NC(=O)N(c2ccccc2C(C)C)C2CC3(C2)CS(=O)(=O)C3)c(OC(F)F)n1]

In embodiments, the compound is

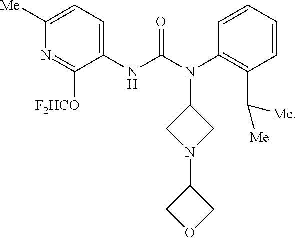 [SMILES: Cc1ccc(NC(=O)N(c2ccccc2C(C)C)C2CN(C3COC3)C2)c(OC(F)F)n1]

In embodiments, the compound is

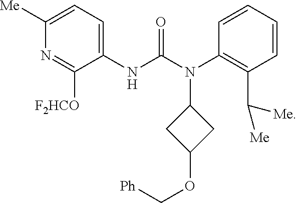 [SMILES: Cc1ccc(NC(=O)N(c2ccccc2C(C)C)C2CC(OCc3ccccc3)C2)c(OC(F)F)n1]

In embodiments, the compound is

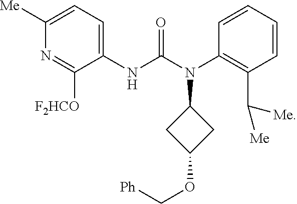 [SMILES: Cc1ccc(NC(=O)N(c2ccccc2C(C)C)[C@H]2C[C@H](OCc3ccccc3)C2)c(OC(F)F)n1]

In embodiments, the compound is

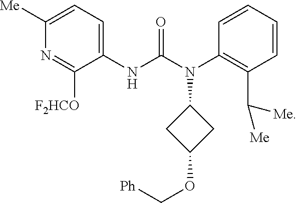 [SMILES: Cc1ccc(NC(=O)N(c2ccccc2C(C)C)[C@H]2C[C@@H](OCc3ccccc3)C2)c(OC(F)F)n1]

In embodiments, the compound is

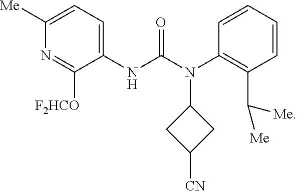 [SMILES: Cc1ccc(NC(=O)N(c2ccccc2C(C)C)C2CC(C#N)C2)c(OC(F)F)n1]

In embodiments, the compound is

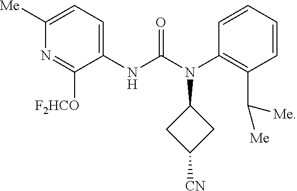 [SMILES: Cc1ccc(NC(=O)N(c2ccccc2C(C)C)[C@H]2C[C@H](C#N)C2)c(OC(F)F)n1]

In embodiments, the compound is

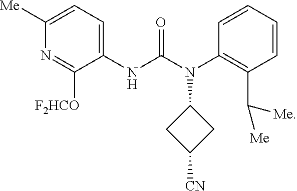 [SMILES: Cc1ccc(NC(=O)N(c2ccccc2C(C)C)[C@H]2C[C@@H](C#N)C2)c(OC(F)F)n1]

In embodiments, the compound is

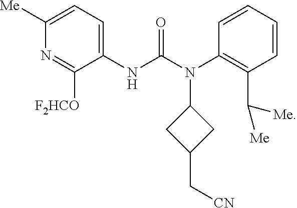 [SMILES: Cc1ccc(NC(=O)N(c2ccccc2C(C)C)C2CC(CC#N)C2)c(OC(F)F)n1]

In embodiments, the compound is

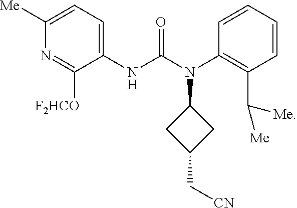 [SMILES: Cc1ccc(NC(=O)N(c2ccccc2C(C)C)[C@H]2C[C@H](CC#N)C2)c(OC(F)F)n1]

In embodiments, the compound is

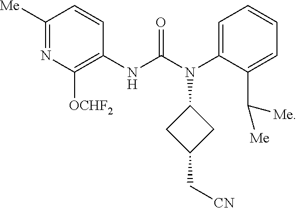 [SMILES: Cc1ccc(NC(=O)N(c2ccccc2C(C)C)[C@H]2C[C@@H](CC#N)C2)c(OC(F)F)n1]

In embodiments, the compound is

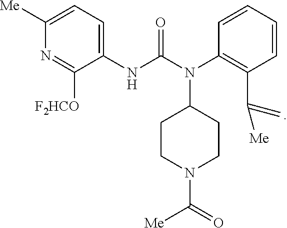 [SMILES: C=C(C)c1ccccc1N(C(=O)Nc1ccc(C)nc1OC(F)F)C1CCN(C(C)=O)CC1]

In embodiments, the compound is

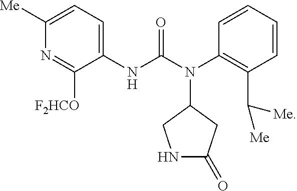 [SMILES: Cc1ccc(NC(=O)N(c2ccccc2C(C)C)C2CNC(=O)C2)c(OC(F)F)n1]

In embodiments, the compound is

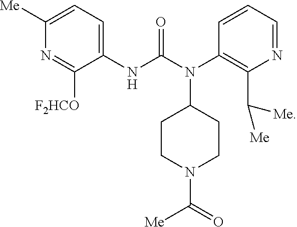 [SMILES: CC(=O)N1CCC(N(C(=O)Nc2ccc(C)nc2OC(F)F)c2cccnc2C(C)C)CC1]

In embodiments, the compound is

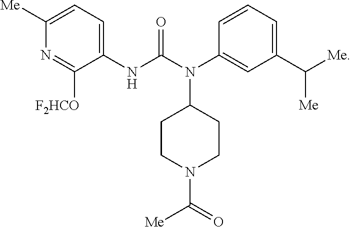 [SMILES: CC(=O)N1CCC(N(C(=O)Nc2ccc(C)nc2OC(F)F)c2cccc(C(C)C)c2)CC1]

In embodiments, the compound is

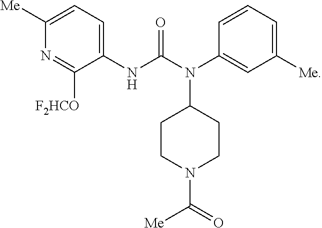 [SMILES: CC(=O)N1CCC(N(C(=O)Nc2ccc(C)nc2OC(F)F)c2cccc(C)c2)CC1]

In embodiments, the compound is

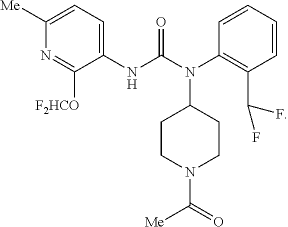 [SMILES: CC(=O)N1CCC(N(C(=O)Nc2ccc(C)nc2OC(F)F)c2ccccc2C(F)F)CC1]

In embodiments, the compound is

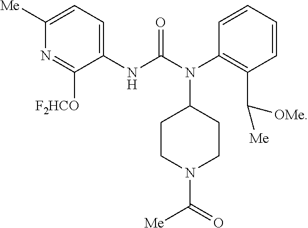 [SMILES: COC(C)c1ccccc1N(C(=O)Nc1ccc(C)nc1OC(F)F)C1CCN(C(C)=O)CC1]

In embodiments, the compound is

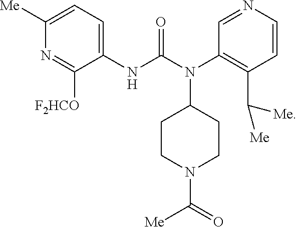 [SMILES: CC(=O)N1CCC(N(C(=O)Nc2ccc(C)nc2OC(F)F)c2cnccc2C(C)C)CC1]

In embodiments, the compound is

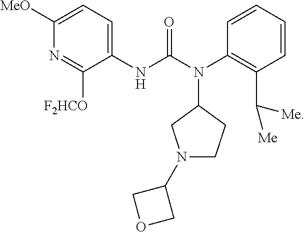 [SMILES: COc1ccc(NC(=O)N(c2ccccc2C(C)C)C2CCN(C3COC3)C2)c(OC(F)F)n1]

In embodiments, the compound is

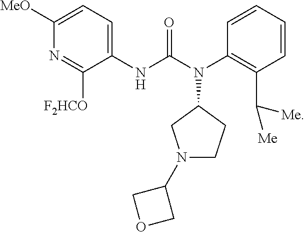 [SMILES: COc1ccc(NC(=O)N(c2ccccc2C(C)C)[C@@H]2CCN(C3COC3)C2)c(OC(F)F)n1]

In embodiments, the compound is

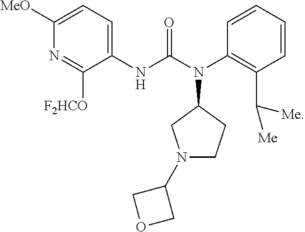 [SMILES: COc1ccc(NC(=O)N(c2ccccc2C(C)C)[C@H]2CCN(C3COC3)C2)c(OC(F)F)n1]

In embodiments, the compound is

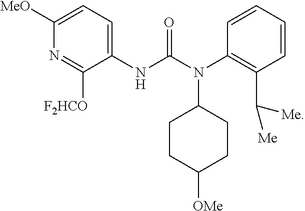 [SMILES: COc1ccc(NC(=O)N(c2ccccc2C(C)C)C2CCC(OC)CC2)c(OC(F)F)n1]

In embodiments, the compound is

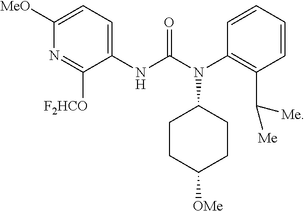 [SMILES: COc1ccc(NC(=O)N(c2ccccc2C(C)C)[C@H]2CC[C@@H](OC)CC2)c(OC(F)F)n1]

In embodiments, the compound is

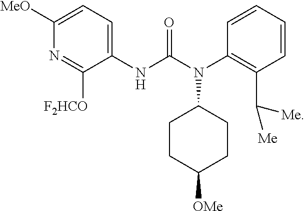 [SMILES: COc1ccc(NC(=O)N(c2ccccc2C(C)C)[C@H]2CC[C@H](OC)CC2)c(OC(F)F)n1]

In embodiments, the compound is

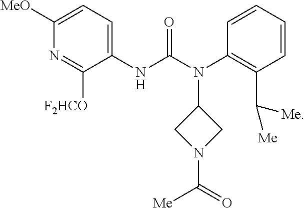 [SMILES: COc1ccc(NC(=O)N(c2ccccc2C(C)C)C2CN(C(C)=O)C2)c(OC(F)F)n1]

In embodiments, the compound is

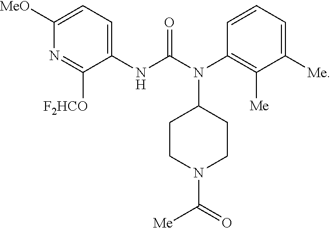 [SMILES: COc1ccc(NC(=O)N(c2cccc(C)c2C)C2CCN(C(C)=O)CC2)c(OC(F)F)n1]

In embodiments, the compound is

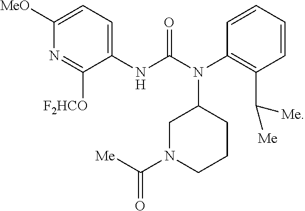 [SMILES: COc1ccc(NC(=O)N(c2ccccc2C(C)C)C2CCCN(C(C)=O)C2)c(OC(F)F)n1]

In embodiments, the compound is

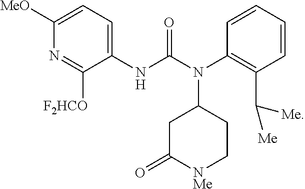 [SMILES: COc1ccc(NC(=O)N(c2ccccc2C(C)C)C2CCN(C)C(=O)C2)c(OC(F)F)n1]

In embodiments, the compound is

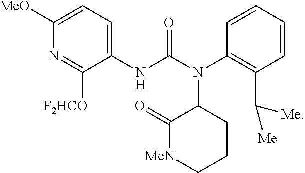 [SMILES: COc1ccc(NC(=O)N(c2ccccc2C(C)C)C2CCCN(C)C2=O)c(OC(F)F)n1]

In embodiments, the compound is

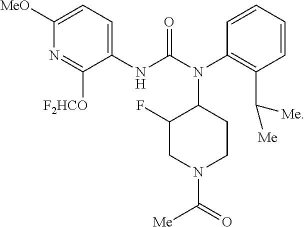 [SMILES: COc1ccc(NC(=O)N(c2ccccc2C(C)C)C2CCN(C(C)=O)CC2F)c(OC(F)F)n1]

In embodiments, the compound is

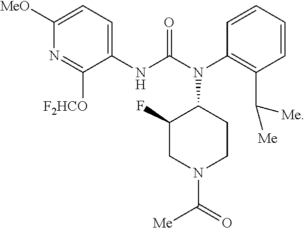 [SMILES: COc1ccc(NC(=O)N(c2ccccc2C(C)C)[C@@H]2CCN(C(C)=O)C[C@H]2F)c(OC(F)F)n1]

In embodiments, the compound is

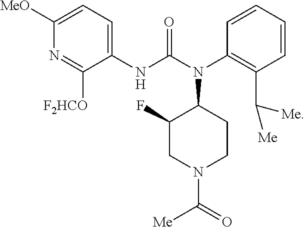 [SMILES: COc1ccc(NC(=O)N(c2ccccc2C(C)C)[C@H]2CCN(C(C)=O)C[C@H]2F)c(OC(F)F)n1]

In embodiments, the compound is

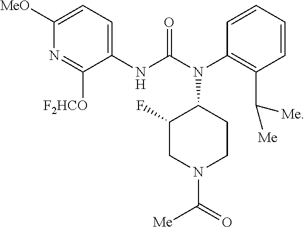 [SMILES: COc1ccc(NC(=O)N(c2ccccc2C(C)C)[C@@H]2CCN(C(C)=O)C[C@@H]2F)c(OC(F)F)n1]

In embodiments, the the compound is

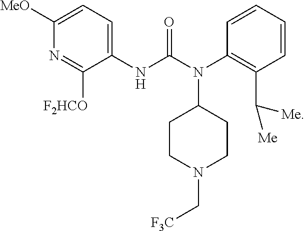 [SMILES: COc1ccc(NC(=O)N(c2ccccc2C(C)C)C2CCN(CC(F)(F)F)CC2)c(OC(F)F)n1]

In embodiments, the compound is

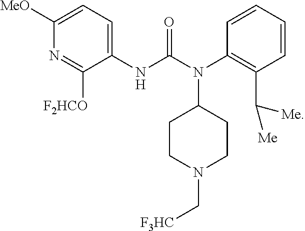

In embodiments, the compound is

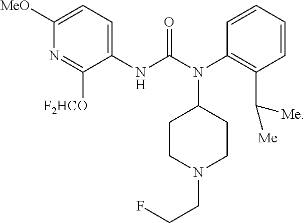 [SMILES: COc1ccc(NC(=O)N(c2ccccc2C(C)C)C2CCN(CCF)CC2)c(OC(F)F)n1]

In embodiments, the compound is

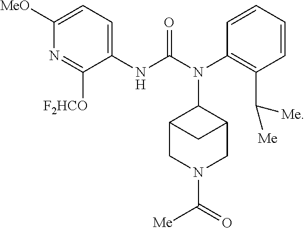 [SMILES: COc1ccc(NC(=O)N(c2ccccc2C(C)C)C2C3CC2CN(C(C)=O)C3)c(OC(F)F)n1]

In embodiments, the compound is

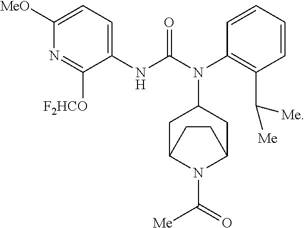 [SMILES: COc1ccc(NC(=O)N(c2ccccc2C(C)C)C2CC3CCC(C2)N3C(C)=O)c(OC(F)F)n1]

In embodiments, the compound is

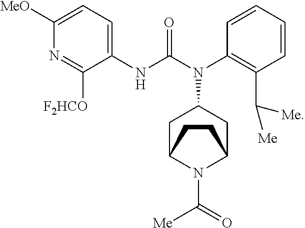

In embodiments, the compound is

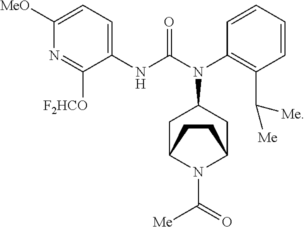

In embodiments, the compound is

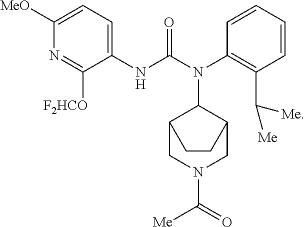 [SMILES: COc1ccc(NC(=O)N(c2ccccc2C(C)C)C2C3CCC2CN(C(C)=O)C3)c(OC(F)F)n1]

In embodiments, the compound is

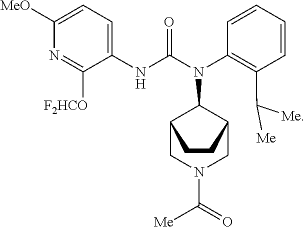

In embodiments, the compound is

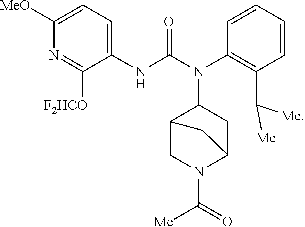 [SMILES: COc1ccc(NC(=O)N(c2ccccc2C(C)C)C2CC3CC2CN3C(C)=O)c(OC(F)F)n1]

In embodiments, the compound is

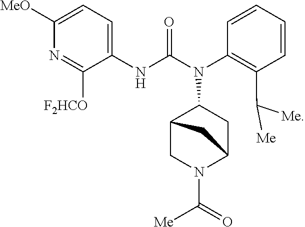 [SMILES: COc1ccc(NC(=O)N(c2ccccc2C(C)C)[C@@H]2C[C@H]3C[C@@H]2CN3C(C)=O)c(OC(F)F)n1]

In embodiments, the compound is

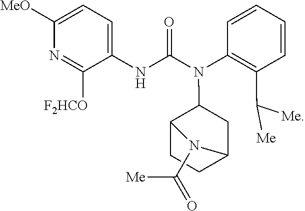 [SMILES: COc1ccc(NC(=O)N(c2ccccc2C(C)C)C2CC3CCC2N3C(C)=O)c(OC(F)F)n1]

In embodiments, the compound is

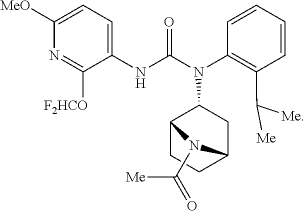 [SMILES: COc1ccc(NC(=O)N(c2ccccc2C(C)C)[C@@H]2C[C@@H]3CC[C@H]2N3C(C)=O)c(OC(F)F)n1]

In embodiments, the compound is

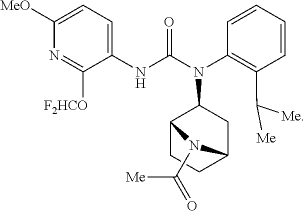 [SMILES: COc1ccc(NC(=O)N(c2ccccc2C(C)C)[C@H]2C[C@@H]3CC[C@H]2N3C(C)=O)c(OC(F)F)n1]

In embodiments, the compound is

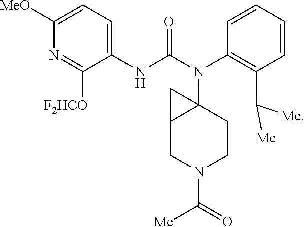 [SMILES: COc1ccc(NC(=O)N(c2ccccc2C(C)C)C23CCN(C(C)=O)CC2C3)c(OC(F)F)n1]

In embodiments, the compound is

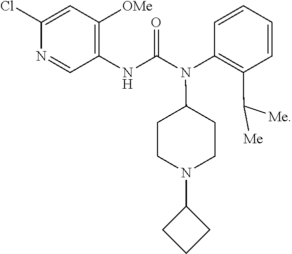 [SMILES: COc1cc(Cl)ncc1NC(=O)N(c1ccccc1C(C)C)C1CCN(C2CCC2)CC1]

In embodiments, the compound is

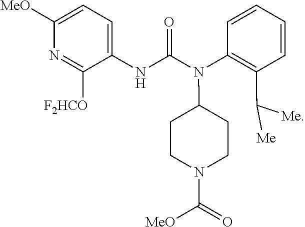 [SMILES: COC(=O)N1CCC(N(C(=O)Nc2ccc(OC)nc2OC(F)F)c2ccccc2C(C)C)CC1]

In embodiments, the compound is

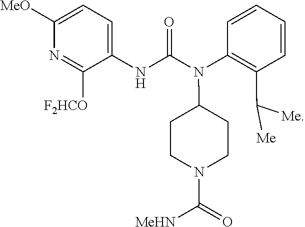 [SMILES: CNC(=O)N1CCC(N(C(=O)Nc2ccc(OC)nc2OC(F)F)c2ccccc2C(C)C)CC1]

In embodiments, the compound is

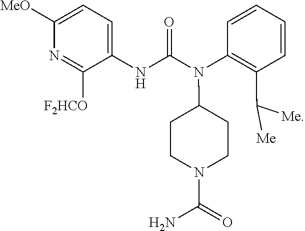 [SMILES: COc1ccc(NC(=O)N(c2ccccc2C(C)C)C2CCN(C(N)=O)CC2)c(OC(F)F)n1]

In embodiments, the compound is

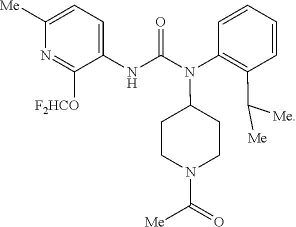 [SMILES: CC(=O)N1CCC(N(C(=O)Nc2ccc(C)nc2OC(F)F)c2ccccc2C(C)C)CC1]

In embodiments, the compound is

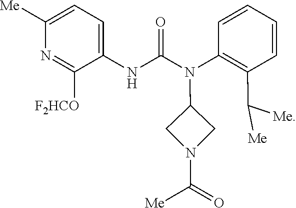 [SMILES: CC(=O)N1CC(N(C(=O)Nc2ccc(C)nc2OC(F)F)c2ccccc2C(C)C)C1]

In embodiments, the compound is

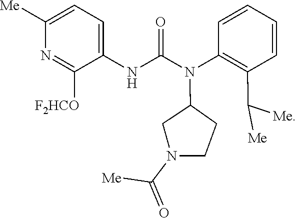 [SMILES: CC(=O)N1CCC(N(C(=O)Nc2ccc(C)nc2OC(F)F)c2ccccc2C(C)C)C1]

In embodiments the compound is

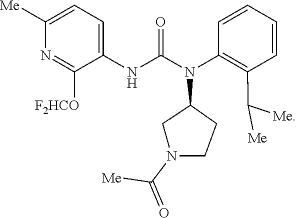 [SMILES: CC(=O)N1CC[C@H](N(C(=O)Nc2ccc(C)nc2OC(F)F)c2ccccc2C(C)C)C1]

In embodiments, the compound is

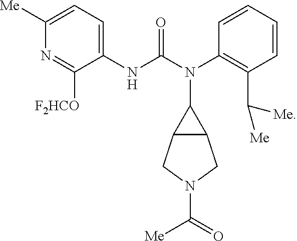 [SMILES: CC(=O)N1CC2C(C1)C2N(C(=O)Nc1ccc(C)nc1OC(F)F)c1ccccc1C(C)C]

In embodiments, the compound is

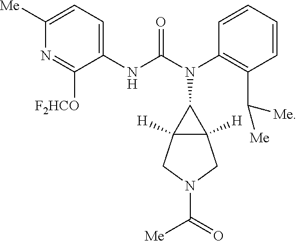 [SMILES: CC(=O)N1C[C@@H]2[C@H](C1)[C@H]2N(C(=O)Nc1ccc(C)nc1OC(F)F)c1ccccc1C(C)C]

In embodiments, the compound is

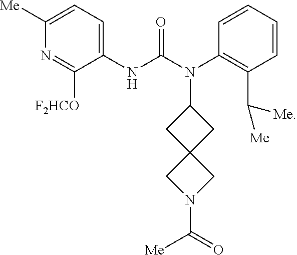 [SMILES: CC(=O)N1CC2(CC(N(C(=O)Nc3ccc(C)nc3OC(F)F)c3ccccc3C(C)C)C2)C1]

In embodiments, the compound is

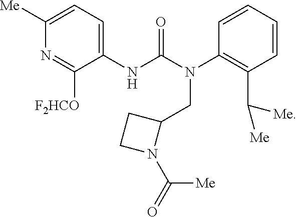 [SMILES: CC(=O)N1CCC1CN(C(=O)Nc1ccc(C)nc1OC(F)F)c1ccccc1C(C)C]

In embodiments, the compound is

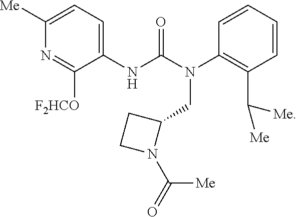 [SMILES: CC(=O)N1CC[C@@H]1CN(C(=O)Nc1ccc(C)nc1OC(F)F)c1ccccc1C(C)C]

In embodiments, the compound is

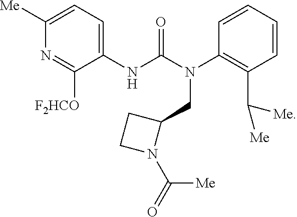 [SMILES: CC(=O)N1CC[C@H]1CN(C(=O)Nc1ccc(C)nc1OC(F)F)c1ccccc1C(C)C]

In embodiments, the compound is

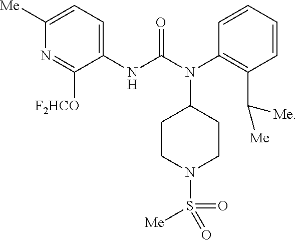 [SMILES: Cc1ccc(NC(=O)N(c2ccccc2C(C)C)C2CCN(S(C)(=O)=O)CC2)c(OC(F)F)n1]

In embodiments, the compound is

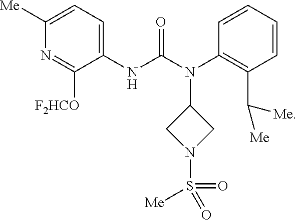 [SMILES: Cc1ccc(NC(=O)N(c2ccccc2C(C)C)C2CN(S(C)(=O)=O)C2)c(OC(F)F)n1]

In embodiments, the compound is

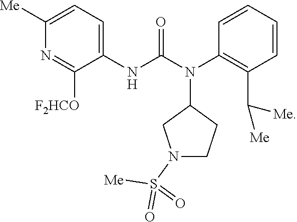 [SMILES: Cc1ccc(NC(=O)N(c2ccccc2C(C)C)C2CCN(S(C)(=O)=O)C2)c(OC(F)F)n1]

In embodiments, the compound is

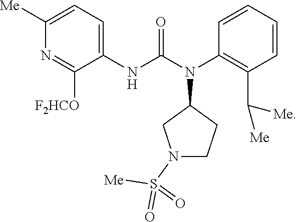 [SMILES: Cc1ccc(NC(=O)N(c2ccccc2C(C)C)[C@H]2CCN(S(C)(=O)=O)C2)c(OC(F)F)n1]

In embodiments, the compound is

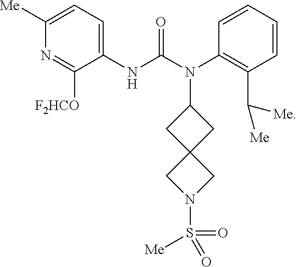 [SMILES: Cc1ccc(NC(=O)N(c2ccccc2C(C)C)C2CC3(C2)CN(S(C)(=O)=O)C3)c(OC(F)F)n1]

In embodiments, the compound is

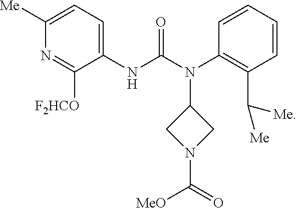 [SMILES: COC(=O)N1CC(N(C(=O)Nc2ccc(C)nc2OC(F)F)c2ccccc2C(C)C)C1]

In embodiments, the compound is

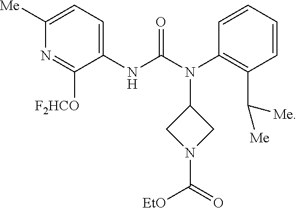 [SMILES: CCOC(=O)N1CC(N(C(=O)Nc2ccc(C)nc2OC(F)F)c2ccccc2C(C)C)C1]

In embodiments, the compound is

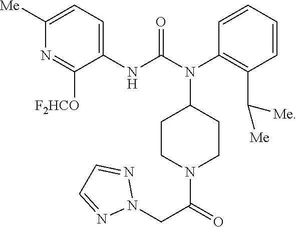 [SMILES: Cc1ccc(NC(=O)N(c2ccccc2C(C)C)C2CCN(C(=O)Cn3nccn3)CC2)c(OC(F)F)n1]

In embodiments, the compound is

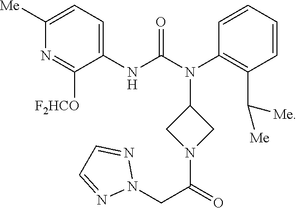 [SMILES: Cc1ccc(NC(=O)N(c2ccccc2C(C)C)C2CN(C(=O)Cn3nccn3)C2)c(OC(F)F)n1]

In embodiments, the compound is

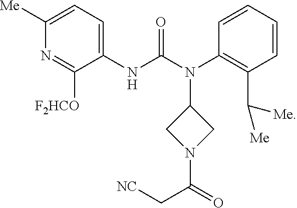 [SMILES: Cc1ccc(NC(=O)N(c2ccccc2C(C)C)C2CN(C(=O)CC#N)C2)c(OC(F)F)n1]

In embodiments, the compound is

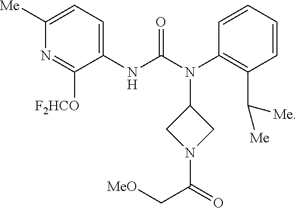 [SMILES: COCC(=O)N1CC(N(C(=O)Nc2ccc(C)nc2OC(F)F)c2ccccc2C(C)C)C1]

In embodiments, the compound is

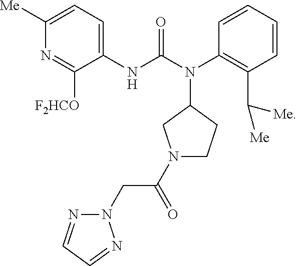 [SMILES: Cc1ccc(NC(=O)N(c2ccccc2C(C)C)C2CCN(C(=O)Cn3nccn3)C2)c(OC(F)F)n1]

In embodiments, the compound is

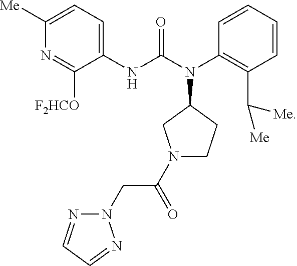 [SMILES: Cc1ccc(NC(=O)N(c2ccccc2C(C)C)[C@H]2CCN(C(=O)Cn3nccn3)C2)c(OC(F)F)n1]

In embodiments, the compound is

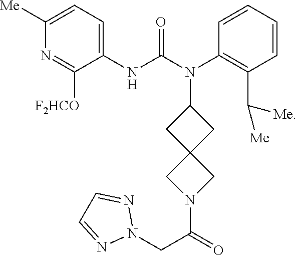 [SMILES: Cc1ccc(NC(=O)N(c2ccccc2C(C)C)C2CC3(C2)CN(C(=O)Cn2nccn2)C3)c(OC(F)F)n1]

In embodiments, the compound is

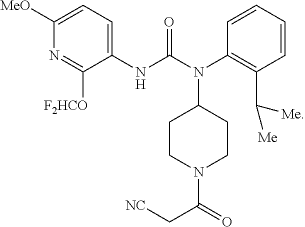 [SMILES: COc1ccc(NC(=O)N(c2ccccc2C(C)C)C2CCN(C(=O)CC#N)CC2)c(OC(F)F)n1]

In embodiments, the compound is

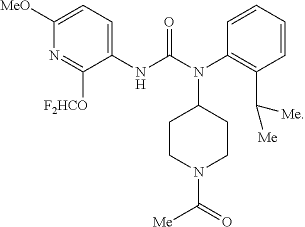 [SMILES: COc1ccc(NC(=O)N(c2ccccc2C(C)C)C2CCN(C(C)=O)CC2)c(OC(F)F)n1]

In embodiments, the compound is

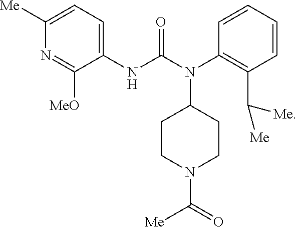 [SMILES: COc1nc(C)ccc1NC(=O)N(c1ccccc1C(C)C)C1CCN(C(C)=O)CC1]

In embodiments, the compound is

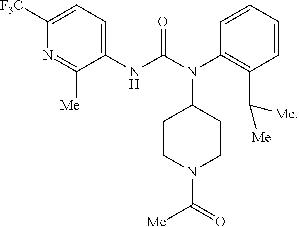 [SMILES: CC(=O)N1CCC(N(C(=O)Nc2ccc(C(F)(F)F)nc2C)c2ccccc2C(C)C)CC1]

In embodiments, the compound is

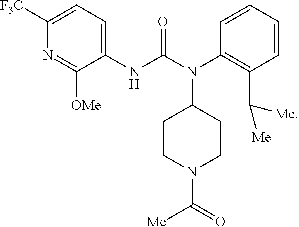 [SMILES: COc1nc(C(F)(F)F)ccc1NC(=O)N(c1ccccc1C(C)C)C1CCN(C(C)=O)CC1]

In embodiments, the compound is

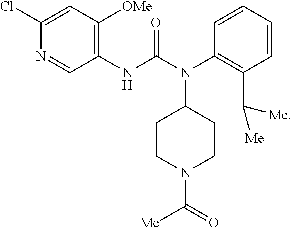 [SMILES: COc1cc(Cl)ncc1NC(=O)N(c1ccccc1C(C)C)C1CCN(C(C)=O)CC1]

In embodiments, the compound is

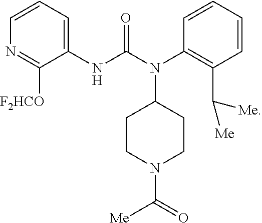 [SMILES: CC(=O)N1CCC(N(C(=O)Nc2cccnc2OC(F)F)c2ccccc2C(C)C)CC1]

In embodiments, the compound is

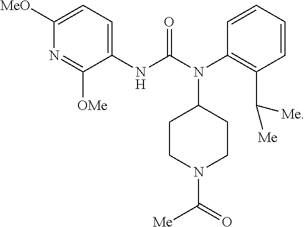 [SMILES: COc1ccc(NC(=O)N(c2ccccc2C(C)C)C2CCN(C(C)=O)CC2)c(OC)n1]

In embodiments, the compound is

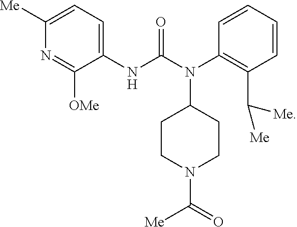 [SMILES: COc1nc(C)ccc1NC(=O)N(c1ccccc1C(C)C)C1CCN(C(C)=O)CC1]

In embodiments, the compound is

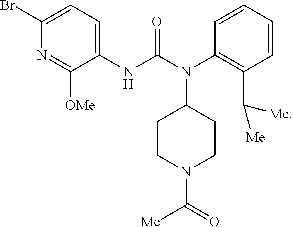 [SMILES: COc1nc(Br)ccc1NC(=O)N(c1ccccc1C(C)C)C1CCN(C(C)=O)CC1]

In embodiments, the compound is

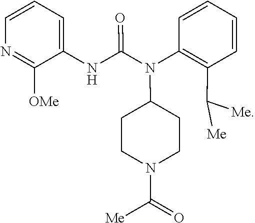 [SMILES: COc1ncccc1NC(=O)N(c1ccccc1C(C)C)C1CCN(C(C)=O)CC1]

In embodiments, the compound is

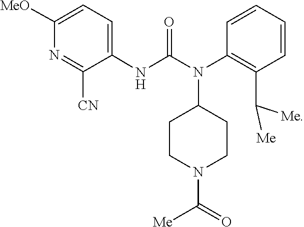 [SMILES: COc1ccc(NC(=O)N(c2ccccc2C(C)C)C2CCN(C(C)=O)CC2)c(C#N)n1]

In embodiments, the compound is

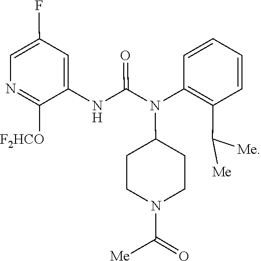 [SMILES: CC(=O)N1CCC(N(C(=O)Nc2cc(F)cnc2OC(F)F)c2ccccc2C(C)C)CC1]

In embodiments, the compound is

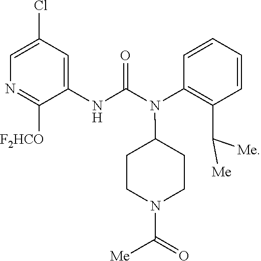 [SMILES: CC(=O)N1CCC(N(C(=O)Nc2cc(Cl)cnc2OC(F)F)c2ccccc2C(C)C)CC1]

In embodiments, the compound is

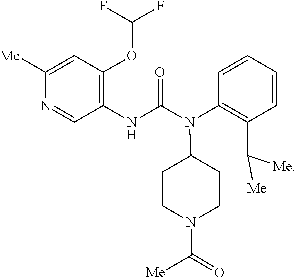 [SMILES: CC(=O)N1CCC(N(C(=O)Nc2cnc(C)cc2OC(F)F)c2ccccc2C(C)C)CC1]

In embodiments, the compound is

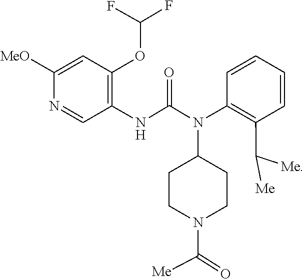 [SMILES: COc1cc(OC(F)F)c(NC(=O)N(c2ccccc2C(C)C)C2CCN(C(C)=O)CC2)cn1]

In embodiments, the compound is

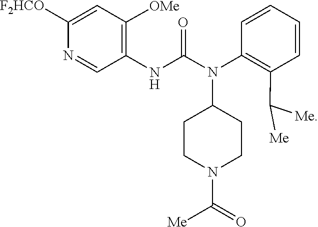 [SMILES: COc1cc(OC(F)F)ncc1NC(=O)N(c1ccccc1C(C)C)C1CCN(C(C)=O)CC1]

In embodiments, the compound is

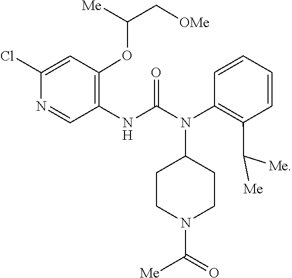 [SMILES: COCC(C)Oc1cc(Cl)ncc1NC(=O)N(c1ccccc1C(C)C)C1CCN(C(C)=O)CC1]

In embodiments, the compound is

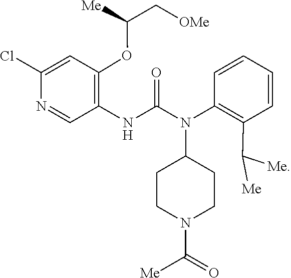 [SMILES: COC[C@H](C)Oc1cc(Cl)ncc1NC(=O)N(c1ccccc1C(C)C)C1CCN(C(C)=O)CC1]

In embodiments, the compound is

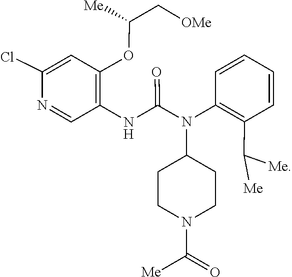 [SMILES: COC[C@@H](C)Oc1cc(Cl)ncc1NC(=O)N(c1ccccc1C(C)C)C1CCN(C(C)=O)CC1]

In embodiments, the compound is

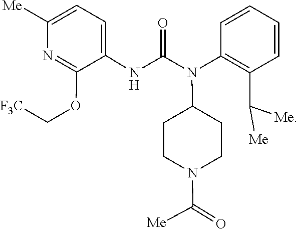 [SMILES: CC(=O)N1CCC(N(C(=O)Nc2ccc(C)nc2OCC(F)(F)F)c2ccccc2C(C)C)CC1]

In embodiments, the compound is

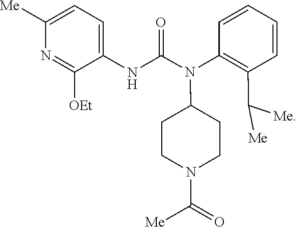 [SMILES: CCOc1nc(C)ccc1NC(=O)N(c1ccccc1C(C)C)C1CCN(C(C)=O)CC1]

In embodiments, the compound is

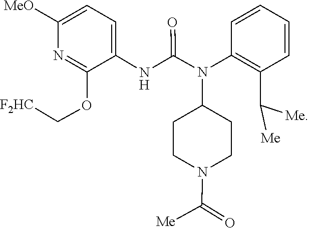 [SMILES: COc1ccc(NC(=O)N(c2ccccc2C(C)C)C2CCN(C(C)=O)CC2)c(OCC(F)F)n1]

In embodiments, the compound is

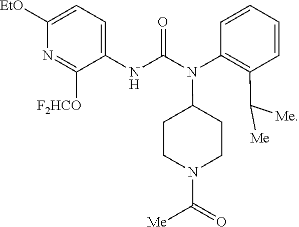 [SMILES: CCOc1ccc(NC(=O)N(c2ccccc2C(C)C)C2CCN(C(C)=O)CC2)c(OC(F)F)n1]

In embodiments, the compound is

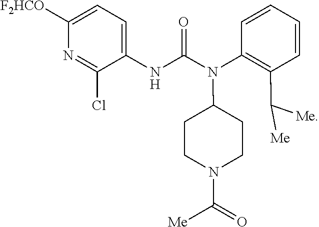 [SMILES: CC(=O)N1CCC(N(C(=O)Nc2ccc(OC(F)F)nc2Cl)c2ccccc2C(C)C)CC1]

In embodiments, the compound is

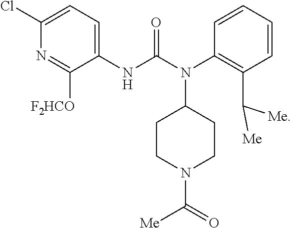 [SMILES: CC(=O)N1CCC(N(C(=O)Nc2ccc(Cl)nc2OC(F)F)c2ccccc2C(C)C)CC1]

In embodiments, the compound is

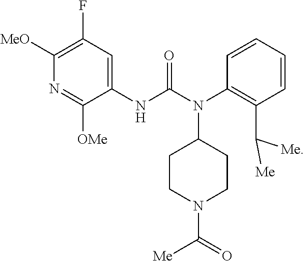 [SMILES: COc1nc(OC)c(NC(=O)N(c2ccccc2C(C)C)C2CCN(C(C)=O)CC2)cc1F]

In embodiments, the compound is

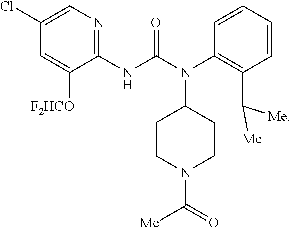 [SMILES: CC(=O)N1CCC(N(C(=O)Nc2ncc(Cl)cc2OC(F)F)c2ccccc2C(C)C)CC1]

In embodiments, the compound is

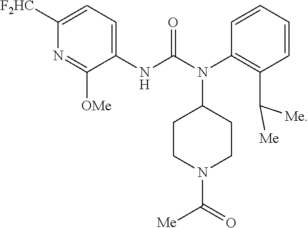 [SMILES: COc1nc(C(F)F)ccc1NC(=O)N(c1ccccc1C(C)C)C1CCN(C(C)=O)CC1]

In embodiments, the compound is

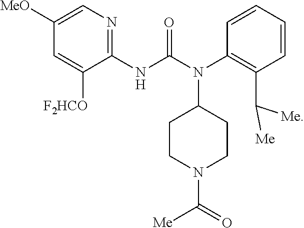 [SMILES: COc1cnc(NC(=O)N(c2ccccc2C(C)C)C2CCN(C(C)=O)CC2)c(OC(F)F)c1]

In embodiments, the compound is

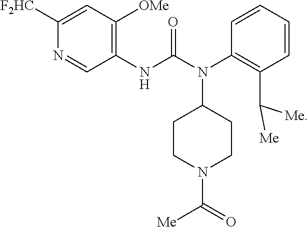 [SMILES: COc1cc(C(F)F)ncc1NC(=O)N(c1ccccc1C(C)C)C1CCN(C(C)=O)CC1]

In embodiments, the compound is

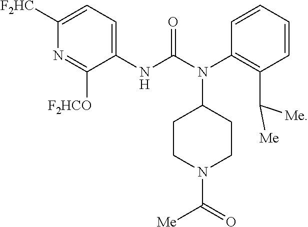 [SMILES: CC(=O)N1CCC(N(C(=O)Nc2ccc(C(F)F)nc2OC(F)F)c2ccccc2C(C)C)CC1]

In embodiments, the compound is

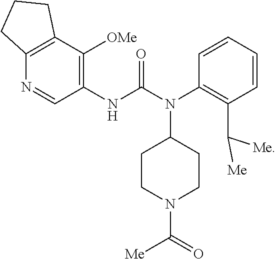 [SMILES: COc1c(NC(=O)N(c2ccccc2C(C)C)C2CCN(C(C)=O)CC2)cnc2c1CCC2]

In embodiments, the compound is

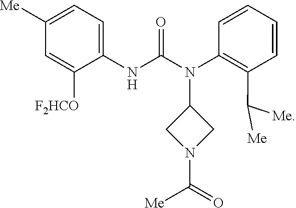 [SMILES: CC(=O)N1CC(N(C(=O)Nc2ccc(C)cc2OC(F)F)c2ccccc2C(C)C)C1]

In embodiments, the compound is

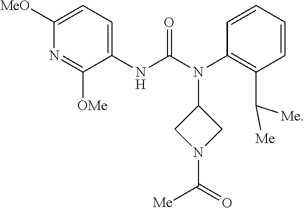 [SMILES: COc1ccc(NC(=O)N(c2ccccc2C(C)C)C2CN(C(C)=O)C2)c(OC)n1]

In embodiments, the compound is

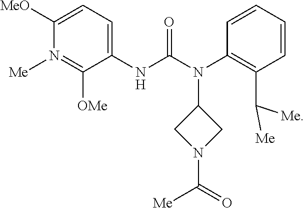

In embodiments, the compound is

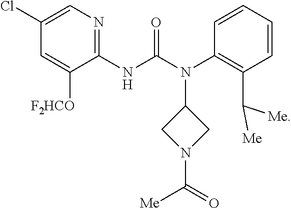 [SMILES: CC(=O)N1CC(N(C(=O)Nc2ncc(Cl)cc2OC(F)F)c2ccccc2C(C)C)C1]

In embodiments, the compound is

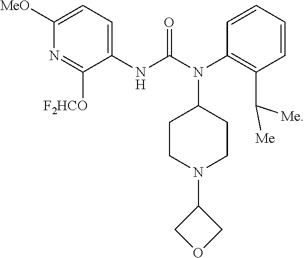 [SMILES: COc1ccc(NC(=O)N(c2ccccc2C(C)C)C2CCN(C3COC3)CC2)c(OC(F)F)n1]

In embodiments, the compound is

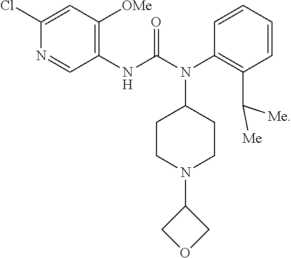 [SMILES: COc1cc(Cl)ncc1NC(=O)N(c1ccccc1C(C)C)C1CCN(C2COC2)CC1]

In embodiments, the compound is

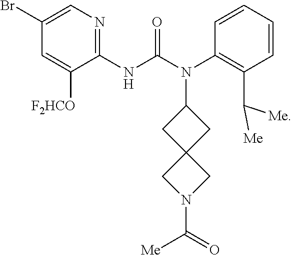 [SMILES: CC(=O)N1CC2(CC(N(C(=O)Nc3ncc(Br)cc3OC(F)F)c3ccccc3C(C)C)C2)C1]

In embodiments, the compound is

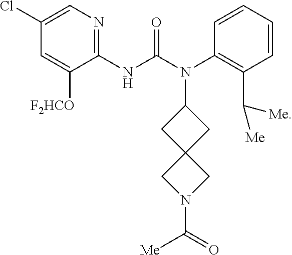 [SMILES: CC(=O)N1CC2(CC(N(C(=O)Nc3ncc(Cl)cc3OC(F)F)c3ccccc3C(C)C)C2)C1]

In embodiments, the compound is

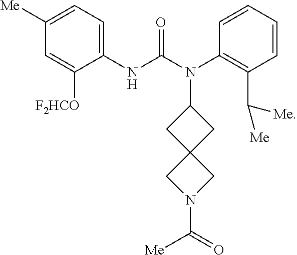 [SMILES: CC(=O)N1CC2(CC(N(C(=O)Nc3ccc(C)cc3OC(F)F)c3ccccc3C(C)C)C2)C1]

In embodiments, the compound is

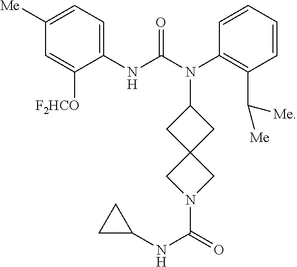 [SMILES: Cc1ccc(NC(=O)N(c2ccccc2C(C)C)C2CC3(C2)CN(C(=O)NC2CC2)C3)c(OC(F)F)c1]

In embodiments, the compound is

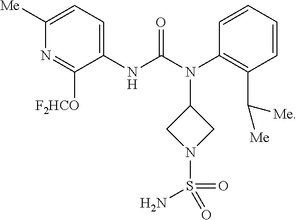 [SMILES: Cc1ccc(NC(=O)N(c2ccccc2C(C)C)C2CN(S(N)(=O)=O)C2)c(OC(F)F)n1]

In embodiments, the compound is

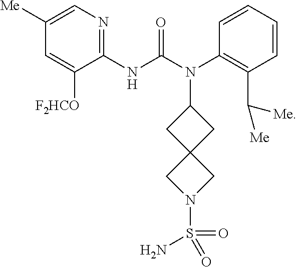 [SMILES: Cc1cnc(NC(=O)N(c2ccccc2C(C)C)C2CC3(C2)CN(S(N)(=O)=O)C3)c(OC(F)F)c1]

In embodiments, the compound is

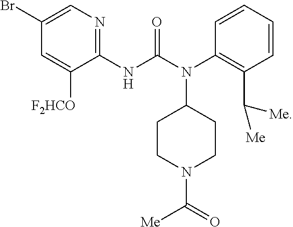 [SMILES: CC(=O)N1CCC(N(C(=O)Nc2ncc(Br)cc2OC(F)F)c2ccccc2C(C)C)CC1]

In embodiments, the compound is

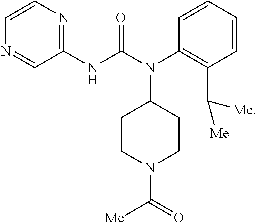 [SMILES: CC(=O)N1CCC(N(C(=O)Nc2cnccn2)c2ccccc2C(C)C)CC1]

In embodiments, the compound is

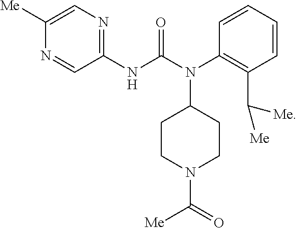 [SMILES: CC(=O)N1CCC(N(C(=O)Nc2cnc(C)cn2)c2ccccc2C(C)C)CC1]

In embodiments, the compound is

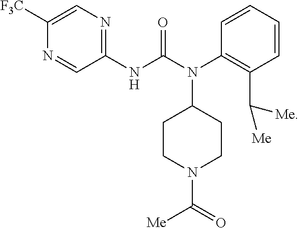 [SMILES: CC(=O)N1CCC(N(C(=O)Nc2cnc(C(F)(F)F)cn2)c2ccccc2C(C)C)CC1]

In embodiments, the compound is

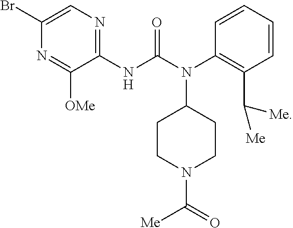 [SMILES: COc1nc(Br)cnc1NC(=O)N(c1ccccc1C(C)C)C1CCN(C(C)=O)CC1]

In embodiments, the compound is

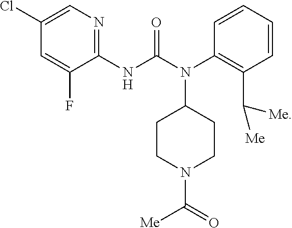 [SMILES: CC(=O)N1CCC(N(C(=O)Nc2ncc(Cl)cc2F)c2ccccc2C(C)C)CC1]

In embodiments, the compound is

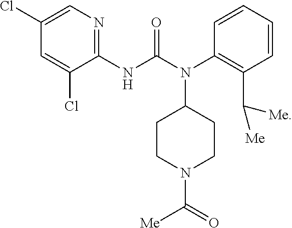 [SMILES: CC(=O)N1CCC(N(C(=O)Nc2ncc(Cl)cc2Cl)c2ccccc2C(C)C)CC1]

In embodiments, the compound is

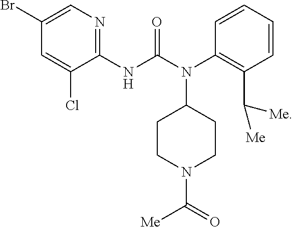 [SMILES: CC(=O)N1CCC(N(C(=O)Nc2ncc(Br)cc2Cl)c2ccccc2C(C)C)CC1]

In embodiments, the compound is

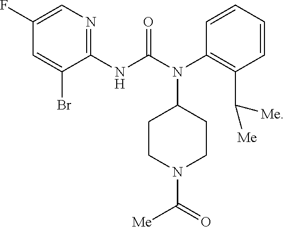 [SMILES: CC(=O)N1CCC(N(C(=O)Nc2ncc(F)cc2Br)c2ccccc2C(C)C)CC1]

In embodiments, the compound is

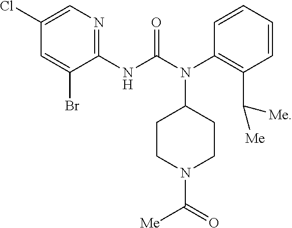 [SMILES: CC(=O)N1CCC(N(C(=O)Nc2ncc(Cl)cc2Br)c2ccccc2C(C)C)CC1]

In embodiments, the compound is

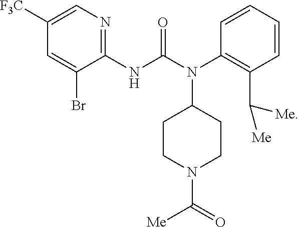 [SMILES: CC(=O)N1CCC(N(C(=O)Nc2ncc(C(F)(F)F)cc2Br)c2ccccc2C(C)C)CC1]

In embodiments, the compound is

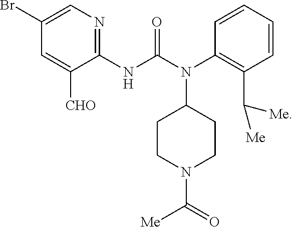 [SMILES: CC(=O)N1CCC(N(C(=O)Nc2ncc(Br)cc2C=O)c2ccccc2C(C)C)CC1]

In embodiments, the compound is

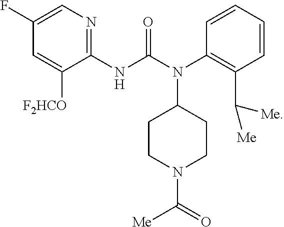 [SMILES: CC(=O)N1CCC(N(C(=O)Nc2ncc(F)cc2OC(F)F)c2ccccc2C(C)C)CC1]

In embodiments, the compound is

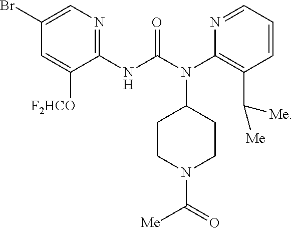 [SMILES: CC(=O)N1CCC(N(C(=O)Nc2ncc(Br)cc2OC(F)F)c2ncccc2C(C)C)CC1]

In embodiments, the compound is

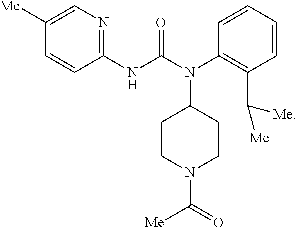 [SMILES: CC(=O)N1CCC(N(C(=O)Nc2ccc(C)cn2)c2ccccc2C(C)C)CC1]

In embodiments, the compound is

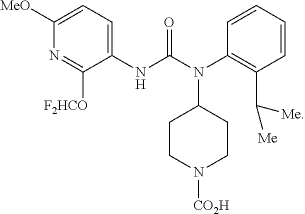 [SMILES: COc1ccc(NC(=O)N(c2ccccc2C(C)C)C2CCN(C(=O)O)CC2)c(OC(F)F)n1]

In embodiments, the compound is

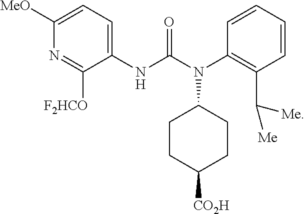 [SMILES: COc1ccc(NC(=O)N(c2ccccc2C(C)C)[C@H]2CC[C@H](C(=O)O)CC2)c(OC(F)F)n1]

In embodiments, the compound is

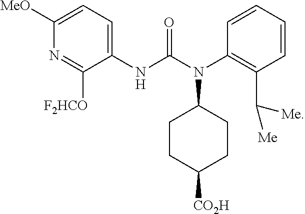 [SMILES: COc1ccc(NC(=O)N(c2ccccc2C(C)C)[C@H]2CC[C@@H](C(=O)O)CC2)c(OC(F)F)n1]

In embodiments, the compound is

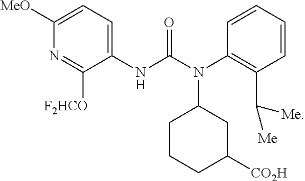 [SMILES: COc1ccc(NC(=O)N(c2ccccc2C(C)C)C2CCCC(C(=O)O)C2)c(OC(F)F)n1]

In embodiments, the compound is

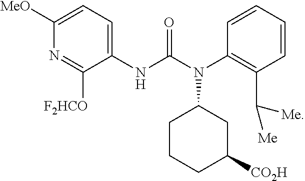 [SMILES: COc1ccc(NC(=O)N(c2ccccc2C(C)C)[C@H]2CCC[C@H](C(=O)O)C2)c(OC(F)F)n1]

In embodiments, the compound is

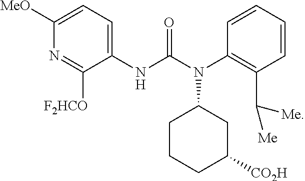 [SMILES: COc1ccc(NC(=O)N(c2ccccc2C(C)C)[C@H]2CCC[C@@H](C(=O)O)C2)c(OC(F)F)n1]

In embodiments, the compound is

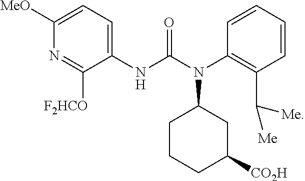 [SMILES: COc1ccc(NC(=O)N(c2ccccc2C(C)C)[C@@H]2CCC[C@H](C(=O)O)C2)c(OC(F)F)n1]

In embodiments, the compound is

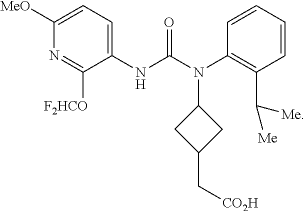 [SMILES: COc1ccc(NC(=O)N(c2ccccc2C(C)C)C2CC(CC(=O)O)C2)c(OC(F)F)n1]

In embodiments, the compound is

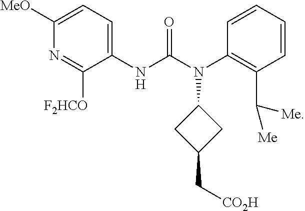 [SMILES: COc1ccc(NC(=O)N(c2ccccc2C(C)C)[C@H]2C[C@H](CC(=O)O)C2)c(OC(F)F)n1]

In embodiments, the compound is

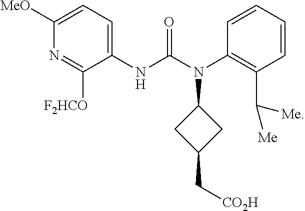 [SMILES: COc1ccc(NC(=O)N(c2ccccc2C(C)C)[C@H]2C[C@@H](CC(=O)O)C2)c(OC(F)F)n1]

In embodiments, the compound is

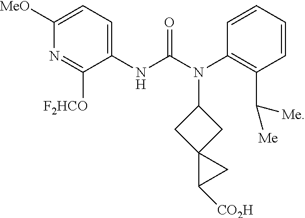 [SMILES: COc1ccc(NC(=O)N(c2ccccc2C(C)C)C2CC3(C2)CC3C(=O)O)c(OC(F)F)n1]

In embodiments, the compound is

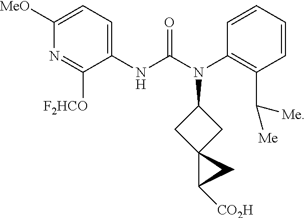 [SMILES: COc1ccc(NC(=O)N(c2ccccc2C(C)C)[C@H]2C[C@@]3(C[C@@H]3C(=O)O)C2)c(OC(F)F)n1]

In embodiments, the compound is

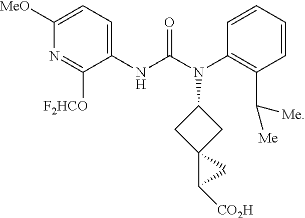 [SMILES: COc1ccc(NC(=O)N(c2ccccc2C(C)C)[C@H]2C[C@@]3(C[C@H]3C(=O)O)C2)c(OC(F)F)n1]

In embodiments, the compound is

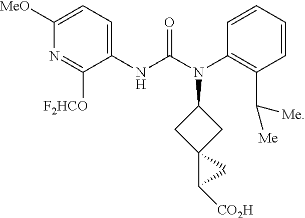 [SMILES: COc1ccc(NC(=O)N(c2ccccc2C(C)C)[C@H]2C[C@]3(C[C@H]3C(=O)O)C2)c(OC(F)F)n1]

In embodiments, the compound is

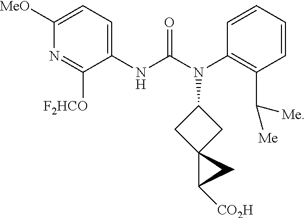 [SMILES: COc1ccc(NC(=O)N(c2ccccc2C(C)C)[C@H]2C[C@]3(C[C@@H]3C(=O)O)C2)c(OC(F)F)n1]

In embodiments, the compound is

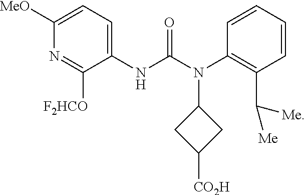 [SMILES: COc1ccc(NC(=O)N(c2ccccc2C(C)C)C2CC(C(=O)O)C2)c(OC(F)F)n1]

In embodiments, the compound is

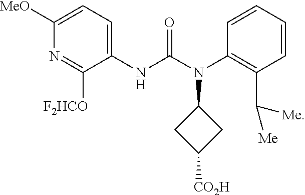 [SMILES: COc1ccc(NC(=O)N(c2ccccc2C(C)C)[C@H]2C[C@H](C(=O)O)C2)c(OC(F)F)n1]

In embodiments, the compound is

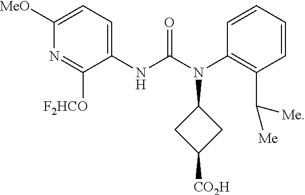 [SMILES: COc1ccc(NC(=O)N(c2ccccc2C(C)C)[C@H]2C[C@@H](C(=O)O)C2)c(OC(F)F)n1]

In embodiments, the compound is

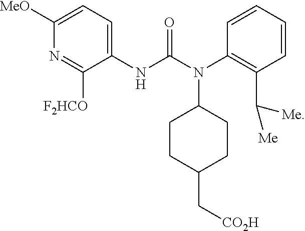 [SMILES: COc1ccc(NC(=O)N(c2ccccc2C(C)C)C2CCC(CC(=O)O)CC2)c(OC(F)F)n1]

In embodiments, the compound is

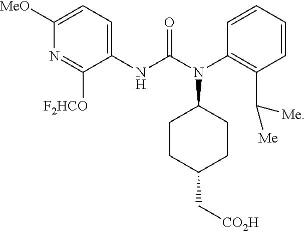 [SMILES: COc1ccc(NC(=O)N(c2ccccc2C(C)C)[C@H]2CC[C@H](CC(=O)O)CC2)c(OC(F)F)n1]

In embodiments, the compound is

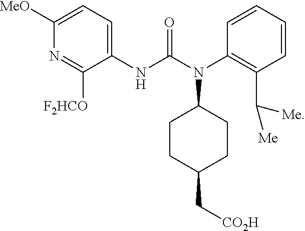 [SMILES: COc1ccc(NC(=O)N(c2ccccc2C(C)C)[C@H]2CC[C@@H](CC(=O)O)CC2)c(OC(F)F)n1]

In embodiments, the compound is

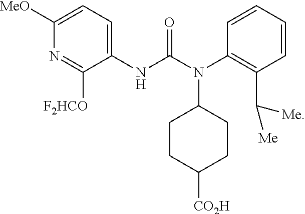 [SMILES: COc1ccc(NC(=O)N(c2ccccc2C(C)C)C2CCC(C(=O)O)CC2)c(OC(F)F)n1]

In embodiments, the compound is

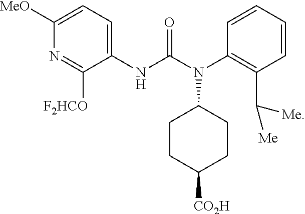 [SMILES: COc1ccc(NC(=O)N(c2ccccc2C(C)C)[C@H]2CC[C@H](C(=O)O)CC2)c(OC(F)F)n1]

In embodiments, the compound is

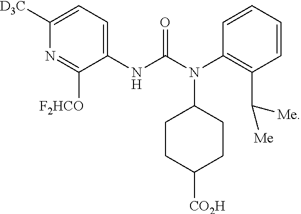 [SMILES: [2H]C([2H])([2H])c1ccc(NC(=O)N(c2ccccc2C(C)C)C2CCC(C(=O)O)CC2)c(OC(F)F)n1]

In embodiments, the compound is

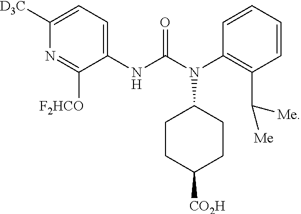 [SMILES: [2H]C([2H])([2H])c1ccc(NC(=O)N(c2ccccc2C(C)C)[C@H]2CC[C@H](C(=O)O)CC2)c(OC(F)F)n1]

In embodiments, the compound is

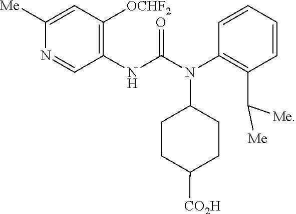 [SMILES: Cc1cc(OC(F)F)c(NC(=O)N(c2ccccc2C(C)C)C2CCC(C(=O)O)CC2)cn1]

In embodiments, the compound is

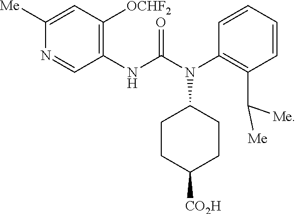 [SMILES: Cc1cc(OC(F)F)c(NC(=O)N(c2ccccc2C(C)C)[C@H]2CC[C@H](C(=O)O)CC2)cn1]

In embodiments, the compound is

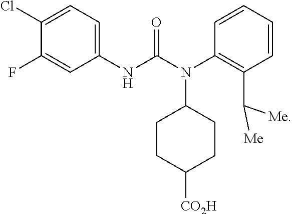 [SMILES: CC(C)c1ccccc1N(C(=O)Nc1ccc(Cl)c(F)c1)C1CCC(C(=O)O)CC1]

In embodiments, the compound is

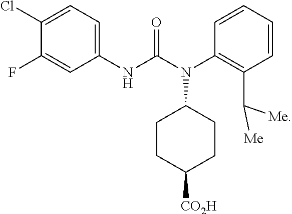 [SMILES: CC(C)c1ccccc1N(C(=O)Nc1ccc(Cl)c(F)c1)[C@H]1CC[C@H](C(=O)O)CC1]

In embodiments, the compound is

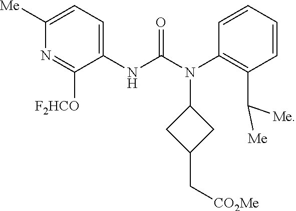 [SMILES: COC(=O)CC1CC(N(C(=O)Nc2ccc(C)nc2OC(F)F)c2ccccc2C(C)C)C1]

In embodiments, the compound is

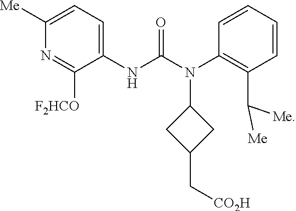 [SMILES: Cc1ccc(NC(=O)N(c2ccccc2C(C)C)C2CC(CC(=O)O)C2)c(OC(F)F)n1]

In embodiments, the compound is

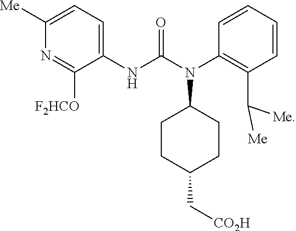 [SMILES: Cc1ccc(NC(=O)N(c2ccccc2C(C)C)[C@H]2CC[C@H](CC(=O)O)CC2)c(OC(F)F)n1]

In embodiments, the compound is

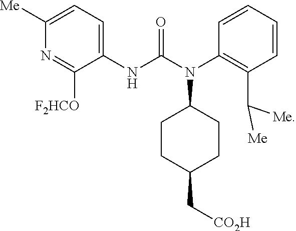 [SMILES: Cc1ccc(NC(=O)N(c2ccccc2C(C)C)[C@H]2CC[C@@H](CC(=O)O)CC2)c(OC(F)F)n1]

In embodiments, the compound is

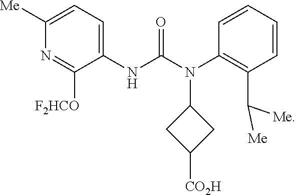 [SMILES: Cc1ccc(NC(=O)N(c2ccccc2C(C)C)C2CC(C(=O)O)C2)c(OC(F)F)n1]

In embodiments, the compound is

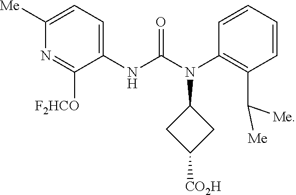 [SMILES: Cc1ccc(NC(=O)N(c2ccccc2C(C)C)[C@H]2C[C@H](C(=O)O)C2)c(OC(F)F)n1]

In embodiments, the compound is

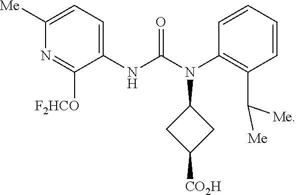 [SMILES: Cc1ccc(NC(=O)N(c2ccccc2C(C)C)[C@H]2C[C@@H](C(=O)O)C2)c(OC(F)F)n1]

In embodiments, the compound is

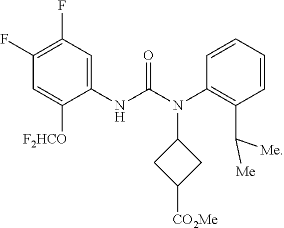 [SMILES: COC(=O)C1CC(N(C(=O)Nc2cc(F)c(F)cc2OC(F)F)c2ccccc2C(C)C)C1]

In embodiments, the compound is

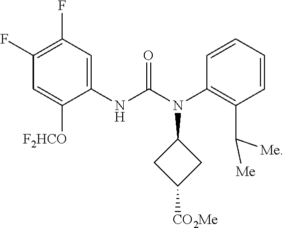 [SMILES: COC(=O)[C@H]1C[C@H](N(C(=O)Nc2cc(F)c(F)cc2OC(F)F)c2ccccc2C(C)C)C1]

In embodiments, the compound is

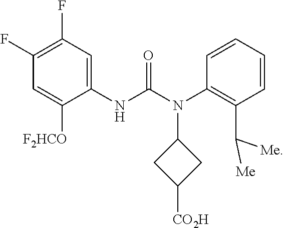 [SMILES: CC(C)c1ccccc1N(C(=O)Nc1cc(F)c(F)cc1OC(F)F)C1CC(C(=O)O)C1]

In embodiments, the compound is

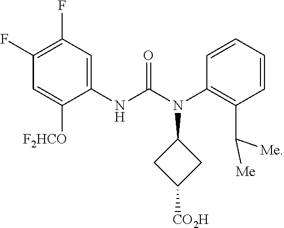 [SMILES: CC(C)c1ccccc1N(C(=O)Nc1cc(F)c(F)cc1OC(F)F)[C@H]1C[C@H](C(=O)O)C1]

In embodiments, the compound is

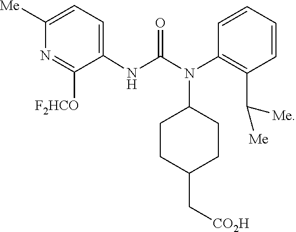 [SMILES: Cc1ccc(NC(=O)N(c2ccccc2C(C)C)C2CCC(CC(=O)O)CC2)c(OC(F)F)n1]

In embodiments, the compound is

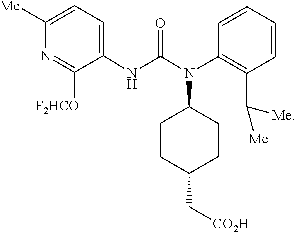 [SMILES: Cc1ccc(NC(=O)N(c2ccccc2C(C)C)[C@H]2CC[C@H](CC(=O)O)CC2)c(OC(F)F)n1]

In embodiments, the compound is

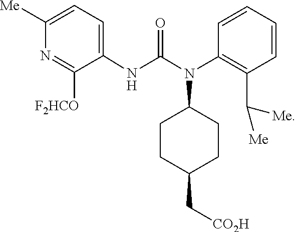 [SMILES: Cc1ccc(NC(=O)N(c2ccccc2C(C)C)[C@H]2CC[C@@H](CC(=O)O)CC2)c(OC(F)F)n1]

In embodiments, the compound is

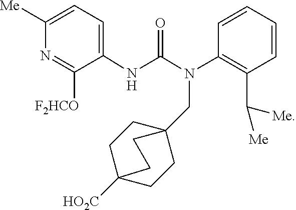 [SMILES: Cc1ccc(NC(=O)N(CC23CCC(C(=O)O)(CC2)CC3)c2ccccc2C(C)C)c(OC(F)F)n1]

In embodiments, the compound is

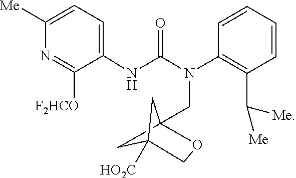 [SMILES: Cc1ccc(NC(=O)N(CC23CC(C(=O)O)(CO2)C3)c2ccccc2C(C)C)c(OC(F)F)n1]

In embodiments, the compound is

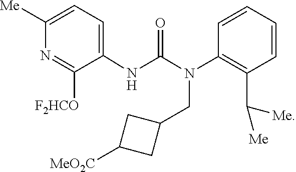 [SMILES: COC(=O)C1CC(CN(C(=O)Nc2ccc(C)nc2OC(F)F)c2ccccc2C(C)C)C1]

In embodiments, the compound is

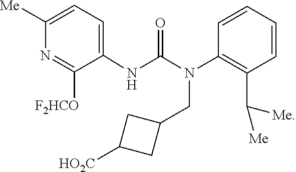 [SMILES: Cc1ccc(NC(=O)N(CC2CC(C(=O)O)C2)c2ccccc2C(C)C)c(OC(F)F)n1]

In embodiments, the compound is

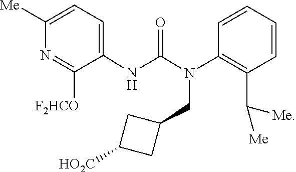 [SMILES: Cc1ccc(NC(=O)N(C[C@H]2C[C@H](C(=O)O)C2)c2ccccc2C(C)C)c(OC(F)F)n1]

In embodiments, the compound is

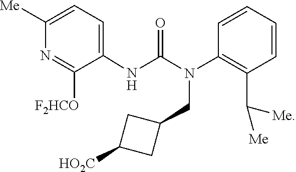 [SMILES: Cc1ccc(NC(=O)N(C[C@H]2C[C@@H](C(=O)O)C2)c2ccccc2C(C)C)c(OC(F)F)n1]

In embodiments, the compound is

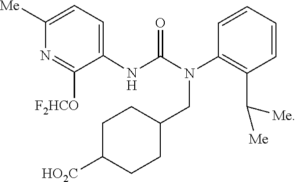 [SMILES: Cc1ccc(NC(=O)N(CC2CCC(C(=O)O)CC2)c2ccccc2C(C)C)c(OC(F)F)n1]

In embodiments, the compound is

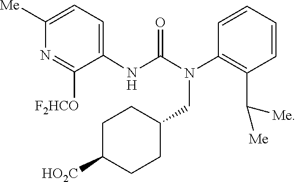 [SMILES: Cc1ccc(NC(=O)N(C[C@H]2CC[C@H](C(=O)O)CC2)c2ccccc2C(C)C)c(OC(F)F)n1]

In embodiments, the compound is

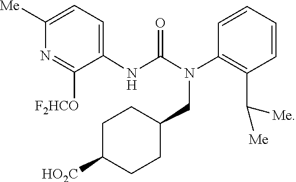 [SMILES: Cc1ccc(NC(=O)N(C[C@H]2CC[C@@H](C(=O)O)CC2)c2ccccc2C(C)C)c(OC(F)F)n1]

In embodiments, the compound is

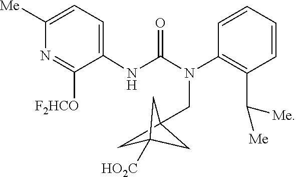 [SMILES: Cc1ccc(NC(=O)N(CC23CC(C(=O)O)(C2)C3)c2ccccc2C(C)C)c(OC(F)F)n1]

In embodiments, the compound is

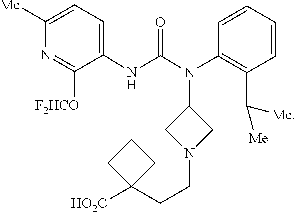 [SMILES: Cc1ccc(NC(=O)N(c2ccccc2C(C)C)C2CN(CCC3(C(=O)O)CCC3)C2)c(OC(F)F)n1]

In embodiments, the compound is

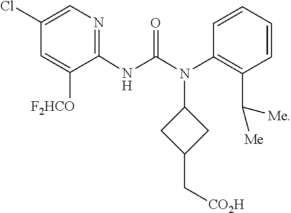 [SMILES: CC(C)c1ccccc1N(C(=O)Nc1ncc(Cl)cc1OC(F)F)C1CC(CC(=O)O)C1]

In embodiments, the compound is

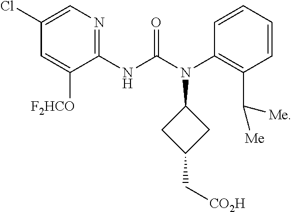 [SMILES: CC(C)c1ccccc1N(C(=O)Nc1ncc(Cl)cc1OC(F)F)[C@H]1C[C@H](CC(=O)O)C1]

In embodiments, the compound is

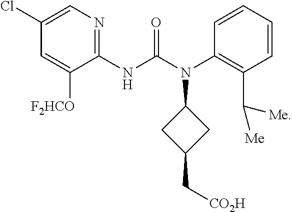 [SMILES: CC(C)c1ccccc1N(C(=O)Nc1ncc(Cl)cc1OC(F)F)[C@H]1C[C@@H](CC(=O)O)C1]

In embodiments, the compound is

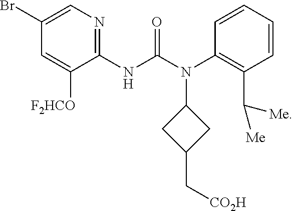 [SMILES: CC(C)c1ccccc1N(C(=O)Nc1ncc(Br)cc1OC(F)F)C1CC(CC(=O)O)C1]

In embodiments, the compound is

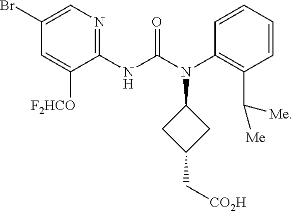 [SMILES: CC(C)c1ccccc1N(C(=O)Nc1ncc(Br)cc1OC(F)F)[C@H]1C[C@H](CC(=O)O)C1]

In embodiments, the compound is

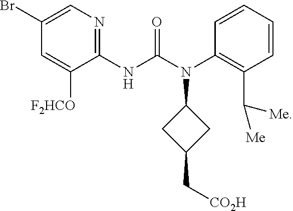 [SMILES: CC(C)c1ccccc1N(C(=O)Nc1ncc(Br)cc1OC(F)F)[C@H]1C[C@@H](CC(=O)O)C1]

In embodiments, the compound is

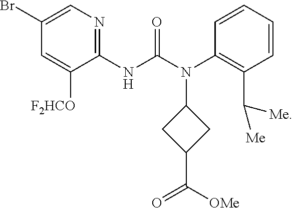 [SMILES: COC(=O)C1CC(N(C(=O)Nc2ncc(Br)cc2OC(F)F)c2ccccc2C(C)C)C1]

In embodiments, the compound is

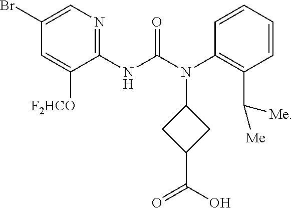 [SMILES: CC(C)c1ccccc1N(C(=O)Nc1ncc(Br)cc1OC(F)F)C1CC(C(=O)O)C1]

In embodiments, the compound is

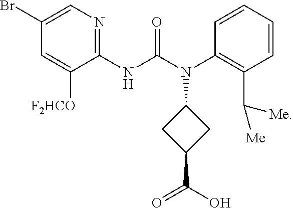 [SMILES: CC(C)c1ccccc1N(C(=O)Nc1ncc(Br)cc1OC(F)F)[C@H]1C[C@H](C(=O)O)C1]

In embodiments, the compound is

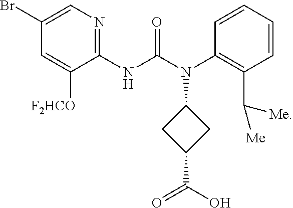 [SMILES: CC(C)c1ccccc1N(C(=O)Nc1ncc(Br)cc1OC(F)F)[C@H]1C[C@@H](C(=O)O)C1]

In embodiments, the compound is

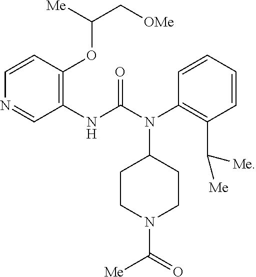 [SMILES: COCC(C)Oc1ccncc1NC(=O)N(c1ccccc1C(C)C)C1CCN(C(C)=O)CC1]

In embodiments, the compound is

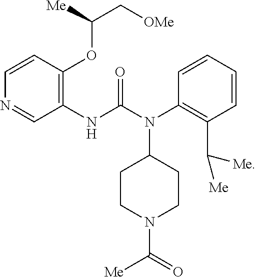 [SMILES: COC[C@H](C)Oc1ccncc1NC(=O)N(c1ccccc1C(C)C)C1CCN(C(C)=O)CC1]

In embodiments, the compound is

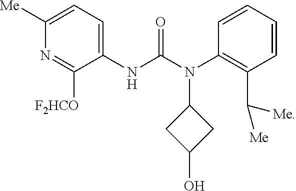 [SMILES: Cc1ccc(NC(=O)N(c2ccccc2C(C)C)C2CC(O)C2)c(OC(F)F)n1]

In embodiments, the compound is

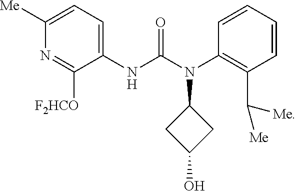 [SMILES: Cc1ccc(NC(=O)N(c2ccccc2C(C)C)[C@H]2C[C@H](O)C2)c(OC(F)F)n1]

In embodiments, the compound is

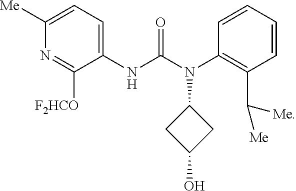 [SMILES: Cc1ccc(NC(=O)N(c2ccccc2C(C)C)[C@H]2C[C@@H](O)C2)c(OC(F)F)n1]

In embodiments, the compound is

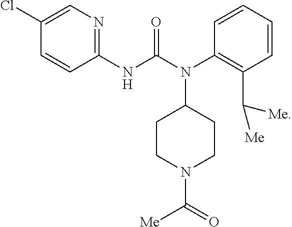 [SMILES: CC(=O)N1CCC(N(C(=O)Nc2ccc(Cl)cn2)c2ccccc2C(C)C)CC1]

In embodiments, the compound is

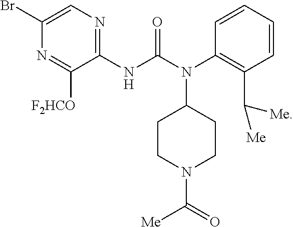 [SMILES: CC(=O)N1CCC(N(C(=O)Nc2ncc(Br)nc2OC(F)F)c2ccccc2C(C)C)CC1]

In embodiments, the compound is

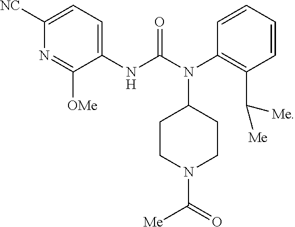 [SMILES: COc1nc(C#N)ccc1NC(=O)N(c1ccccc1C(C)C)C1CCN(C(C)=O)CC1]

In embodiments, the compound is

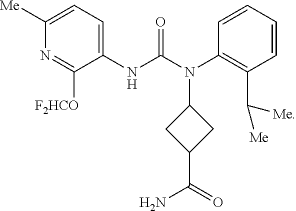 [SMILES: Cc1ccc(NC(=O)N(c2ccccc2C(C)C)C2CC(C(N)=O)C2)c(OC(F)F)n1]

In embodiments, the compound is

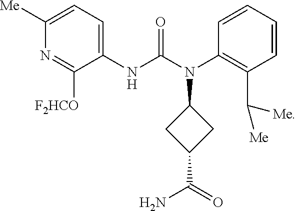 [SMILES: Cc1ccc(NC(=O)N(c2ccccc2C(C)C)[C@H]2C[C@H](C(N)=O)C2)c(OC(F)F)n1]

In embodiments, the compound is

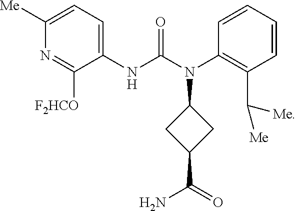 [SMILES: Cc1ccc(NC(=O)N(c2ccccc2C(C)C)[C@H]2C[C@@H](C(N)=O)C2)c(OC(F)F)n1]

In embodiments, the compound is

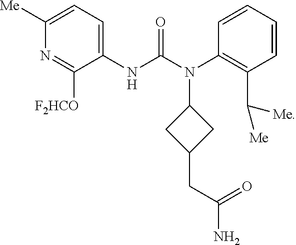 [SMILES: Cc1ccc(NC(=O)N(c2ccccc2C(C)C)C2CC(CC(N)=O)C2)c(OC(F)F)n1]

In embodiments, the compound is

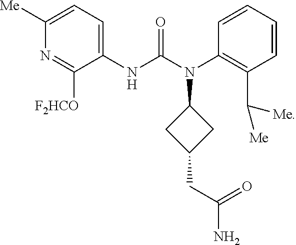 [SMILES: Cc1ccc(NC(=O)N(c2ccccc2C(C)C)[C@H]2C[C@H](CC(N)=O)C2)c(OC(F)F)n1]

In embodiments, the compound is

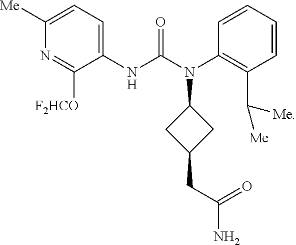 [SMILES: Cc1ccc(NC(=O)N(c2ccccc2C(C)C)[C@H]2C[C@@H](CC(N)=O)C2)c(OC(F)F)n1]

In embodiments, the compound is

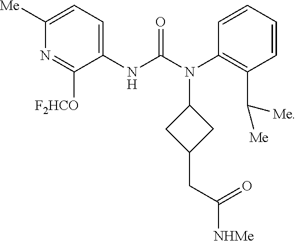 [SMILES: CNC(=O)CC1CC(N(C(=O)Nc2ccc(C)nc2OC(F)F)c2ccccc2C(C)C)C1]

In embodiments, the compound is

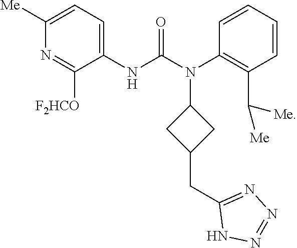 [SMILES: Cc1ccc(NC(=O)N(c2ccccc2C(C)C)C2CC(Cc3nnn[nH]3)C2)c(OC(F)F)n1]

In embodiments, the compound is

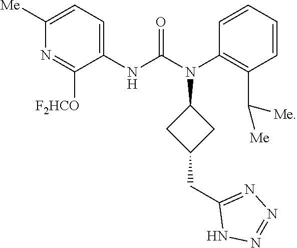 [SMILES: Cc1ccc(NC(=O)N(c2ccccc2C(C)C)[C@H]2C[C@H](Cc3nnn[nH]3)C2)c(OC(F)F)n1]

In embodiments, the compound is

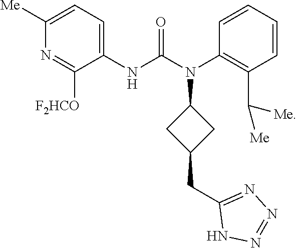 [SMILES: Cc1ccc(NC(=O)N(c2ccccc2C(C)C)[C@H]2C[C@@H](Cc3nnn[nH]3)C2)c(OC(F)F)n1]

In embodiments, the compound is

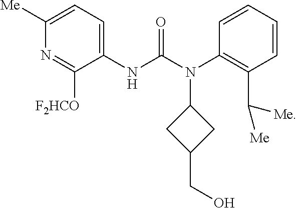 [SMILES: Cc1ccc(NC(=O)N(c2ccccc2C(C)C)C2CC(CO)C2)c(OC(F)F)n1]

In embodiments, the compound is

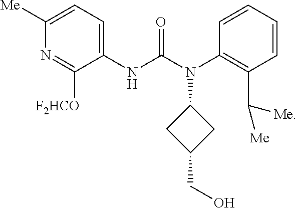 [SMILES: Cc1ccc(NC(=O)N(c2ccccc2C(C)C)[C@H]2C[C@@H](CO)C2)c(OC(F)F)n1]

In embodiments, the compound is

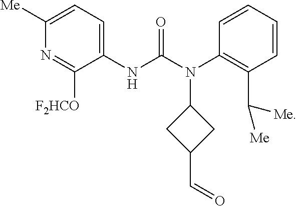 [SMILES: Cc1ccc(NC(=O)N(c2ccccc2C(C)C)C2CC(C=O)C2)c(OC(F)F)n1]

In embodiments, the compound is

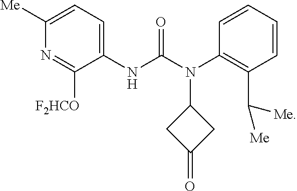 [SMILES: Cc1ccc(NC(=O)N(c2ccccc2C(C)C)C2CC(=O)C2)c(OC(F)F)n1]

In embodiments, the compound is

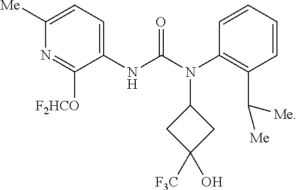 [SMILES: Cc1ccc(NC(=O)N(c2ccccc2C(C)C)C2CC(O)(C(F)(F)F)C2)c(OC(F)F)n1]

In embodiments, the compound is

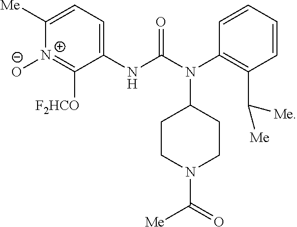 [SMILES: CC(=O)N1CCC(N(C(=O)Nc2ccc(C)[n+]([O-])c2OC(F)F)c2ccccc2C(C)C)CC1]

In embodiments, the compound is

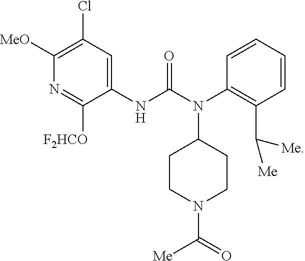 [SMILES: COc1nc(OC(F)F)c(NC(=O)N(c2ccccc2C(C)C)C2CCN(C(C)=O)CC2)cc1Cl]

In embodiments, the compound is

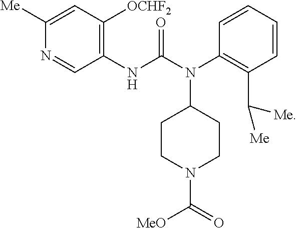 [SMILES: COC(=O)N1CCC(N(C(=O)Nc2cnc(C)cc2OC(F)F)c2ccccc2C(C)C)CC1]

In embodiments, the compound is

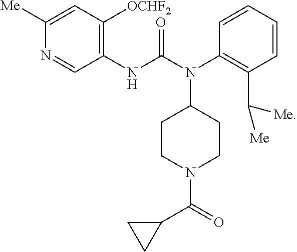 [SMILES: Cc1cc(OC(F)F)c(NC(=O)N(c2ccccc2C(C)C)C2CCN(C(=O)C3CC3)CC2)cn1]

In embodiments, the compound is

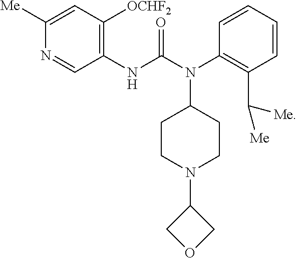 [SMILES: Cc1cc(OC(F)F)c(NC(=O)N(c2ccccc2C(C)C)C2CCN(C3COC3)CC2)cn1]

In embodiments, the compound is

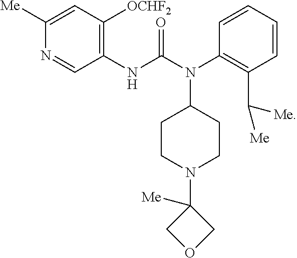 [SMILES: Cc1cc(OC(F)F)c(NC(=O)N(c2ccccc2C(C)C)C2CCN(C3(C)COC3)CC2)cn1]

In embodiments, the compound is

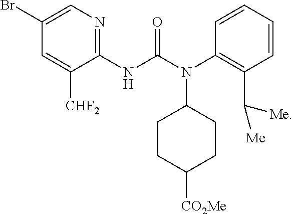 [SMILES: COC(=O)C1CCC(N(C(=O)Nc2ncc(Br)cc2C(F)F)c2ccccc2C(C)C)CC1]

In embodiments, the compound is

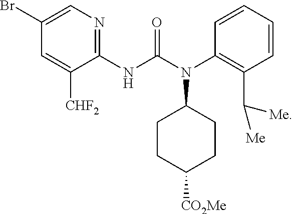 [SMILES: COC(=O)[C@H]1CC[C@H](N(C(=O)Nc2ncc(Br)cc2C(F)F)c2ccccc2C(C)C)CC1]

In embodiments, the compound is

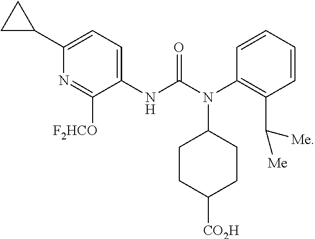 [SMILES: CC(C)c1ccccc1N(C(=O)Nc1ccc(C2CC2)nc1OC(F)F)C1CCC(C(=O)O)CC1]

In embodiments, the compound is

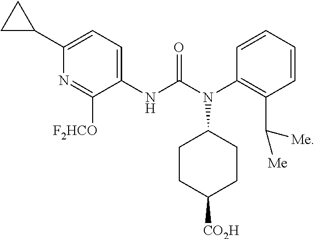 [SMILES: CC(C)c1ccccc1N(C(=O)Nc1ccc(C2CC2)nc1OC(F)F)[C@H]1CC[C@H](C(=O)O)CC1]

In embodiments, the compound is

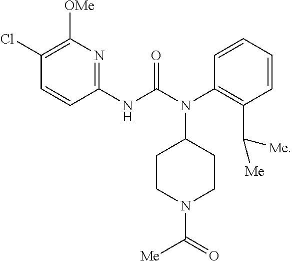 [SMILES: COc1nc(NC(=O)N(c2ccccc2C(C)C)C2CCN(C(C)=O)CC2)ccc1Cl]

In embodiments, the compound is

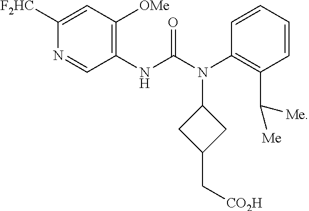 [SMILES: COc1cc(C(F)F)ncc1NC(=O)N(c1ccccc1C(C)C)C1CC(CC(=O)O)C1]

In embodiments, the compound is

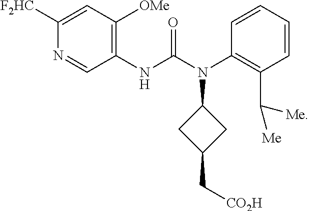 [SMILES: COc1cc(C(F)F)ncc1NC(=O)N(c1ccccc1C(C)C)[C@H]1C[C@@H](CC(=O)O)C1]

In embodiments, the compound is

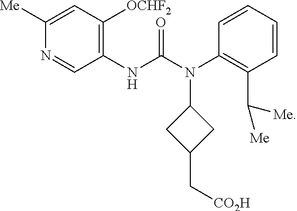 [SMILES: Cc1cc(OC(F)F)c(NC(=O)N(c2ccccc2C(C)C)C2CC(CC(=O)O)C2)cn1]

In embodiments, the compound is

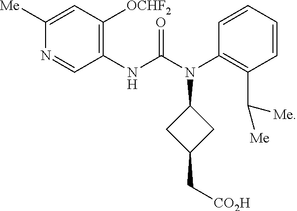 [SMILES: Cc1cc(OC(F)F)c(NC(=O)N(c2ccccc2C(C)C)[C@H]2C[C@@H](CC(=O)O)C2)cn1]

In embodiments, the compound is

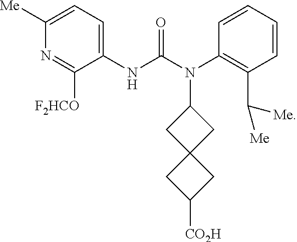 [SMILES: Cc1ccc(NC(=O)N(c2ccccc2C(C)C)C2CC3(CC(C(=O)O)C3)C2)c(OC(F)F)n1]

In embodiments, the compound is

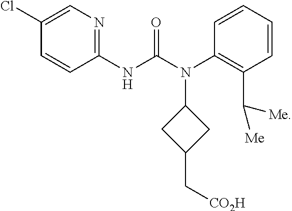 [SMILES: CC(C)c1ccccc1N(C(=O)Nc1ccc(Cl)cn1)C1CC(CC(=O)O)C1]

In embodiments, the compound is

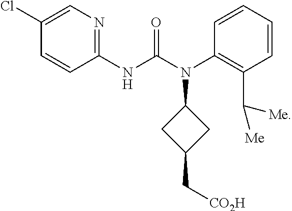 [SMILES: CC(C)c1ccccc1N(C(=O)Nc1ccc(Cl)cn1)[C@H]1C[C@@H](CC(=O)O)C1]

In embodiments, the compound is

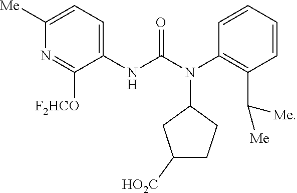 [SMILES: Cc1ccc(NC(=O)N(c2ccccc2C(C)C)C2CCC(C(=O)O)C2)c(OC(F)F)n1]

In embodiments, the compound is

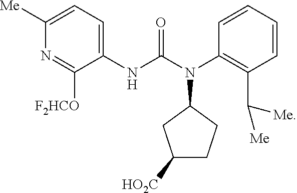 [SMILES: Cc1ccc(NC(=O)N(c2ccccc2C(C)C)[C@H]2CC[C@@H](C(=O)O)C2)c(OC(F)F)n1]

In embodiments, the compound is

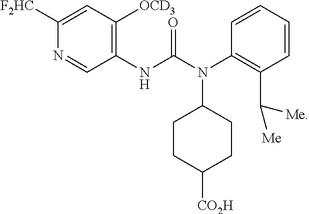

In embodiments, the compound is

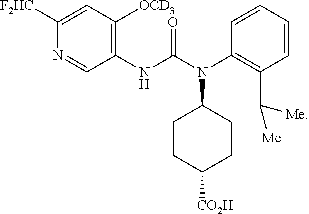

In embodiments, the compound is

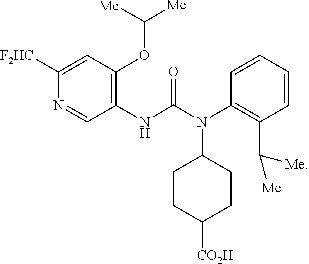 [SMILES: CC(C)Oc1cc(C(F)F)ncc1NC(=O)N(c1ccccc1C(C)C)C1CCC(C(=O)O)CC1]

In embodiments, the compound is

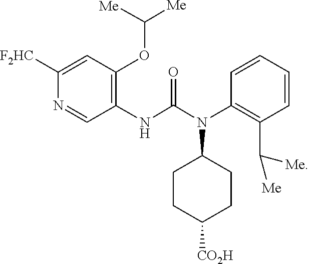 [SMILES: CC(C)Oc1cc(C(F)F)ncc1NC(=O)N(c1ccccc1C(C)C)[C@H]1CC[C@H](C(=O)O)CC1]

In embodiments, the compound is

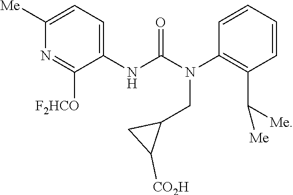 [SMILES: Cc1ccc(NC(=O)N(CC2CC2C(=O)O)c2ccccc2C(C)C)c(OC(F)F)n1]

In embodiments, the compound is

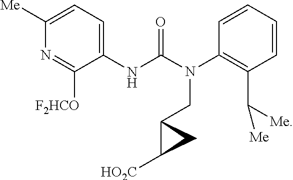 [SMILES: Cc1ccc(NC(=O)N(C[C@@H]2C[C@H]2C(=O)O)c2ccccc2C(C)C)c(OC(F)F)n1]

In embodiments, the compound is

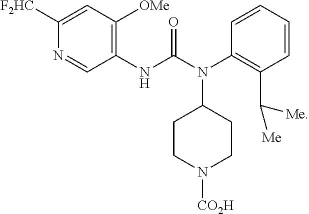 [SMILES: COc1cc(C(F)F)ncc1NC(=O)N(c1ccccc1C(C)C)C1CCN(C(=O)O)CC1]

In embodiments, the compound is

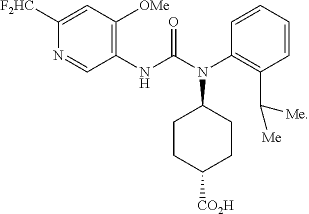

In embodiments, the compound is

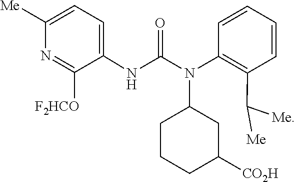 [SMILES: Cc1ccc(NC(=O)N(c2ccccc2C(C)C)C2CCCC(C(=O)O)C2)c(OC(F)F)n1]

In embodiments, the compound is

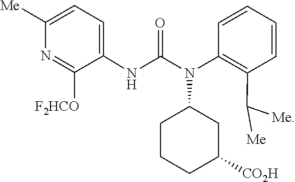 [SMILES: Cc1ccc(NC(=O)N(c2ccccc2C(C)C)[C@H]2CCC[C@@H](C(=O)O)C2)c(OC(F)F)n1]

In embodiments, the compound is

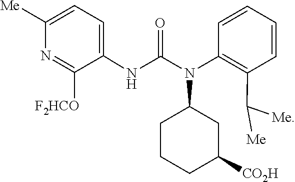 [SMILES: Cc1ccc(NC(=O)N(c2ccccc2C(C)C)[C@@H]2CCC[C@H](C(=O)O)C2)c(OC(F)F)n1]

In embodiments, the compound is

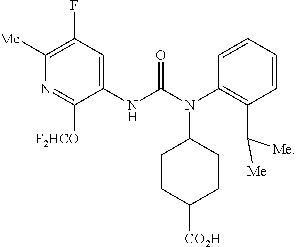 [SMILES: Cc1nc(OC(F)F)c(NC(=O)N(c2ccccc2C(C)C)C2CCC(C(=O)O)CC2)cc1F]

In embodiments, the compound is

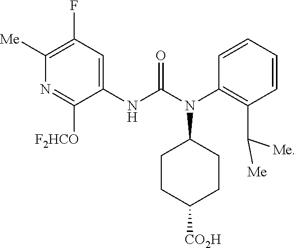

In embodiments, the compound is

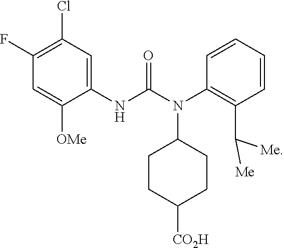 [SMILES: COc1cc(F)c(Cl)cc1NC(=O)N(c1ccccc1C(C)C)C1CCC(C(=O)O)CC1]

In embodiments, the compound is

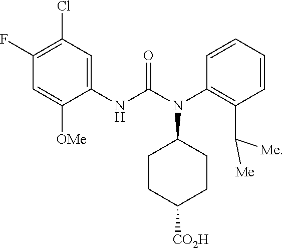

In embodiments, the compound is

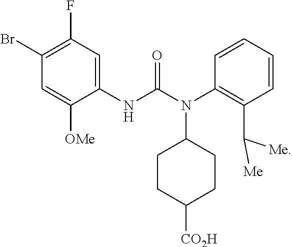 [SMILES: COc1cc(Br)c(F)cc1NC(=O)N(c1ccccc1C(C)C)C1CCC(C(=O)O)CC1]

In embodiments, the compound is

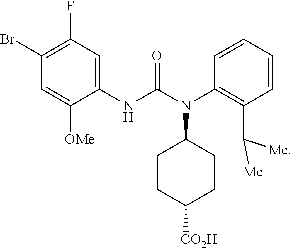

In embodiments, the compound is

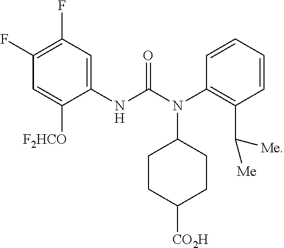 [SMILES: CC(C)c1ccccc1N(C(=O)Nc1cc(F)c(F)cc1OC(F)F)C1CCC(C(=O)O)CC1]

In embodiments, the compound is

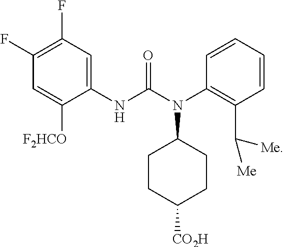 [SMILES: CC(C)c1ccccc1N(C(=O)Nc1cc(F)c(F)cc1OC(F)F)[C@H]1CC[C@H](C(=O)O)CC1]

In embodiments, the compound is

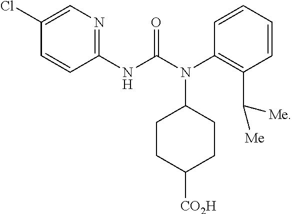 [SMILES: CC(C)c1ccccc1N(C(=O)Nc1ccc(Cl)cn1)C1CCC(C(=O)O)CC1]

In embodiments, the compound is

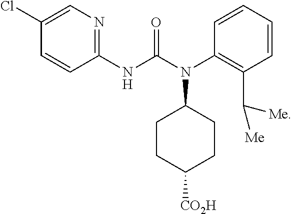 [SMILES: CC(C)c1ccccc1N(C(=O)Nc1ccc(Cl)cn1)[C@H]1CC[C@H](C(=O)O)CC1]

In embodiments, the compound is

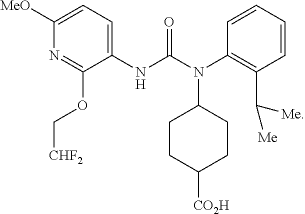 [SMILES: COc1ccc(NC(=O)N(c2ccccc2C(C)C)C2CCC(C(=O)O)CC2)c(OCC(F)F)n1]

In embodiments, the compound is

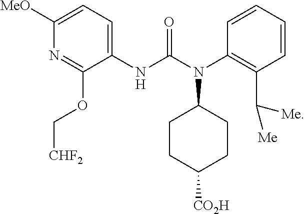 [SMILES: COc1ccc(NC(=O)N(c2ccccc2C(C)C)[C@H]2CC[C@H](C(=O)O)CC2)c(OCC(F)F)n1]

In embodiments, the compound is

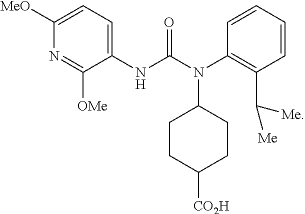 [SMILES: COc1ccc(NC(=O)N(c2ccccc2C(C)C)C2CCC(C(=O)O)CC2)c(OC)n1]

In embodiments, the compound is

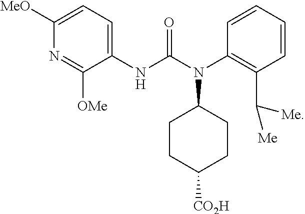 [SMILES: COc1ccc(NC(=O)N(c2ccccc2C(C)C)[C@H]2CC[C@H](C(=O)O)CC2)c(OC)n1]

In embodiments, the compound is

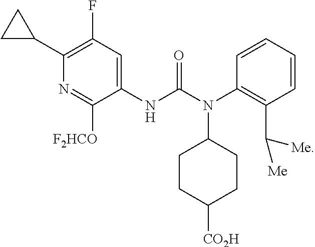 [SMILES: CC(C)c1ccccc1N(C(=O)Nc1cc(F)c(C2CC2)nc1OC(F)F)C1CCC(C(=O)O)CC1]

In embodiments, the compound is

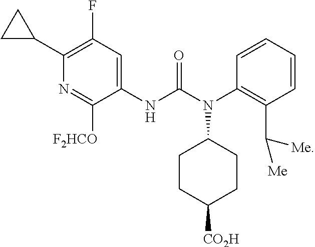 [SMILES: CC(C)c1ccccc1N(C(=O)Nc1cc(F)c(C2CC2)nc1OC(F)F)[C@H]1CC[C@H](C(=O)O)CC1]

In embodiments, the compound is

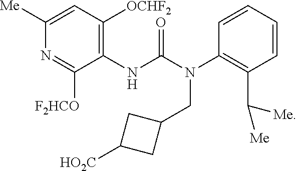 [SMILES: Cc1cc(OC(F)F)c(NC(=O)N(CC2CC(C(=O)O)C2)c2ccccc2C(C)C)c(OC(F)F)n1]

In embodiments, the compound is

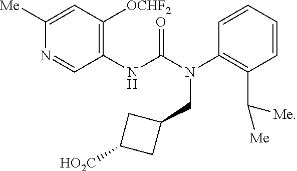 [SMILES: Cc1cc(OC(F)F)c(NC(=O)N(C[C@H]2C[C@H](C(=O)O)C2)c2ccccc2C(C)C)cn1]

In embodiments, the compound is

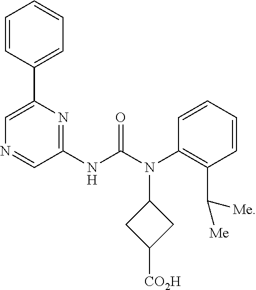 [SMILES: CC(C)c1ccccc1N(C(=O)Nc1cncc(-c2ccccc2)n1)C1CC(C(=O)O)C1]

In embodiments, the compound is

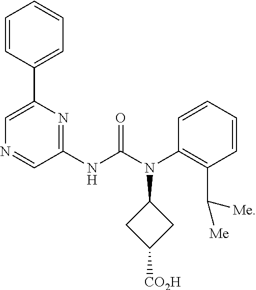 [SMILES: CC(C)c1ccccc1N(C(=O)Nc1cncc(-c2ccccc2)n1)[C@H]1C[C@H](C(=O)O)C1]

In embodiments, the compound is

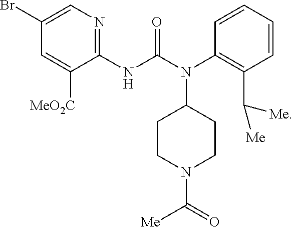 [SMILES: COC(=O)c1cc(Br)cnc1NC(=O)N(c1ccccc1C(C)C)C1CCN(C(C)=O)CC1]

In embodiments, the compound is

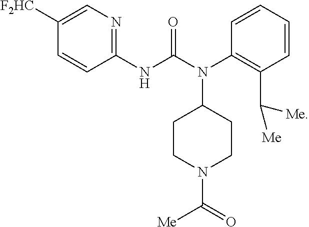 [SMILES: CC(=O)N1CCC(N(C(=O)Nc2ccc(C(F)F)cn2)c2ccccc2C(C)C)CC1]

In embodiments, the compound is

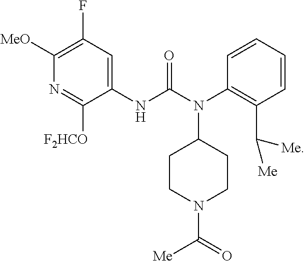 [SMILES: COc1nc(OC(F)F)c(NC(=O)N(c2ccccc2C(C)C)C2CCN(C(C)=O)CC2)cc1F]

In embodiments, the compound is

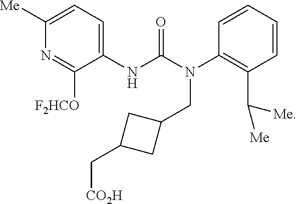 [SMILES: Cc1ccc(NC(=O)N(CC2CC(CC(=O)O)C2)c2ccccc2C(C)C)c(OC(F)F)n1]

In embodiments, the compound is

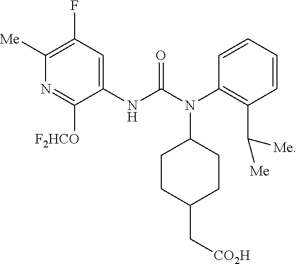 [SMILES: Cc1nc(OC(F)F)c(NC(=O)N(c2ccccc2C(C)C)C2CCC(CC(=O)O)CC2)cc1F]

In embodiments, the compound is

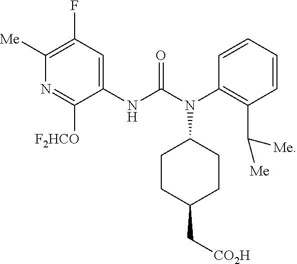 [SMILES: Cc1nc(OC(F)F)c(NC(=O)N(c2ccccc2C(C)C)[C@H]2CC[C@H](CC(=O)O)CC2)cc1F]

In embodiments, the compound is

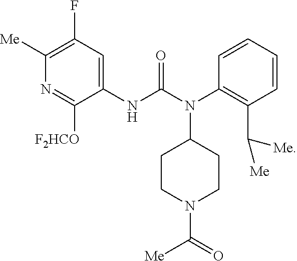 [SMILES: CC(=O)N1CCC(N(C(=O)Nc2cc(F)c(C)nc2OC(F)F)c2ccccc2C(C)C)CC1]

In embodiments, the compound is

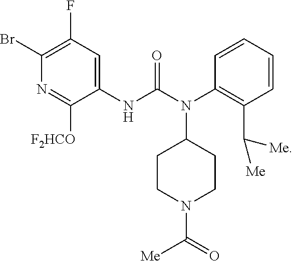 [SMILES: CC(=O)N1CCC(N(C(=O)Nc2cc(F)c(Br)nc2OC(F)F)c2ccccc2C(C)C)CC1]

In embodiments, the compound is

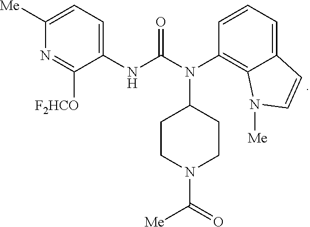 [SMILES: CC(=O)N1CCC(N(C(=O)Nc2ccc(C)nc2OC(F)F)c2cccc3ccn(C)c23)CC1]

In embodiments, the compound is

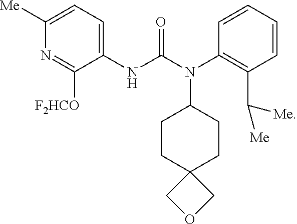 [SMILES: Cc1ccc(NC(=O)N(c2ccccc2C(C)C)C2CCC3(CC2)COC3)c(OC(F)F)n1]

In embodiments, the compound is

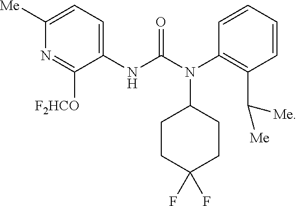 [SMILES: Cc1ccc(NC(=O)N(c2ccccc2C(C)C)C2CCC(F)(F)CC2)c(OC(F)F)n1]

In embodiments, the compound is

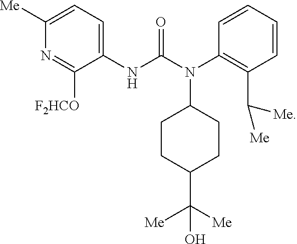 [SMILES: Cc1ccc(NC(=O)N(c2ccccc2C(C)C)C2CCC(C(C)(C)O)CC2)c(OC(F)F)n1]

In embodiments, the compound is

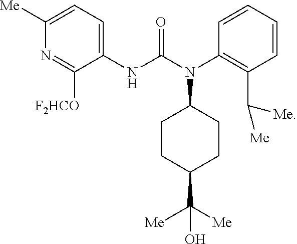 [SMILES: Cc1ccc(NC(=O)N(c2ccccc2C(C)C)[C@H]2CC[C@@H](C(C)(C)O)CC2)c(OC(F)F)n1]

In embodiments, the compound is

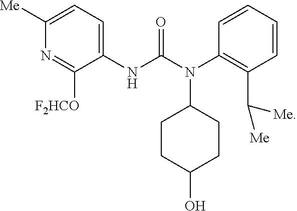 [SMILES: Cc1ccc(NC(=O)N(c2ccccc2C(C)C)C2CCC(O)CC2)c(OC(F)F)n1]

In embodiments, the compound is

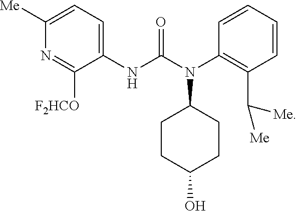 [SMILES: Cc1ccc(NC(=O)N(c2ccccc2C(C)C)[C@H]2CC[C@H](O)CC2)c(OC(F)F)n1]

In embodiments, the compound is

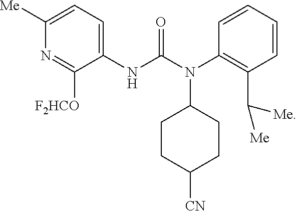 [SMILES: Cc1ccc(NC(=O)N(c2ccccc2C(C)C)C2CCC(C#N)CC2)c(OC(F)F)n1]

In embodiments, the compound is

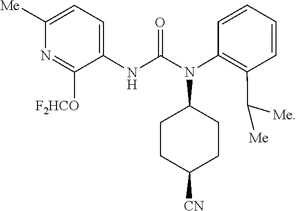 [SMILES: Cc1ccc(NC(=O)N(c2ccccc2C(C)C)[C@H]2CC[C@@H](C#N)CC2)c(OC(F)F)n1]

In embodiments, the compound is

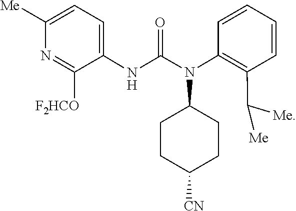 [SMILES: Cc1ccc(NC(=O)N(c2ccccc2C(C)C)[C@H]2CC[C@H](C#N)CC2)c(OC(F)F)n1]

In embodiments, the compound is

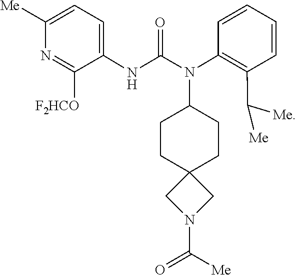 [SMILES: CC(=O)N1CC2(CCC(N(C(=O)Nc3ccc(C)nc3OC(F)F)c3ccccc3C(C)C)CC2)C1]

In embodiments, the compound is

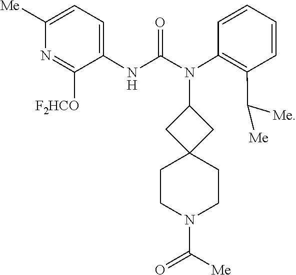 [SMILES: CC(=O)N1CCC2(CC1)CC(N(C(=O)Nc1ccc(C)nc1OC(F)F)c1ccccc1C(C)C)C2]

In embodiments, the compound is

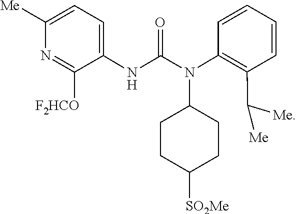 [SMILES: Cc1ccc(NC(=O)N(c2ccccc2C(C)C)C2CCC(S(C)(=O)=O)CC2)c(OC(F)F)n1]

In embodiments, the compound is

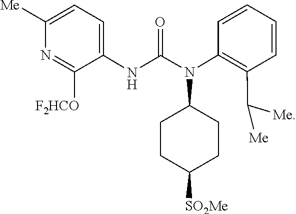 [SMILES: Cc1ccc(NC(=O)N(c2ccccc2C(C)C)[C@H]2CC[C@@H](S(C)(=O)=O)CC2)c(OC(F)F)n1]

In embodiments, the compound is

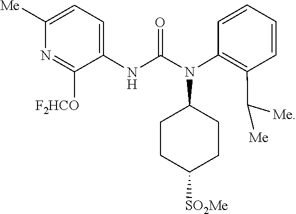 [SMILES: Cc1ccc(NC(=O)N(c2ccccc2C(C)C)[C@H]2CC[C@H](S(C)(=O)=O)CC2)c(OC(F)F)n1]

In embodiments, the compound is

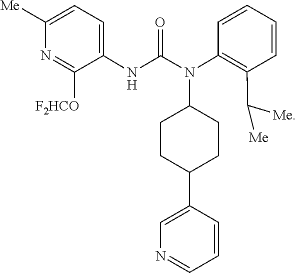 [SMILES: Cc1ccc(NC(=O)N(c2ccccc2C(C)C)C2CCC(c3cccnc3)CC2)c(OC(F)F)n1]

In embodiments, the compound is

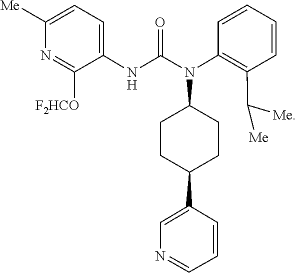 [SMILES: Cc1ccc(NC(=O)N(c2ccccc2C(C)C)[C@H]2CC[C@@H](c3cccnc3)CC2)c(OC(F)F)n1]

In embodiments, the compound is

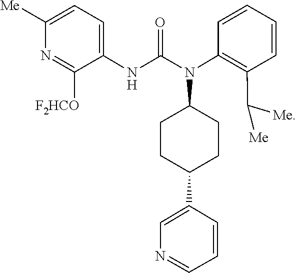 [SMILES: Cc1ccc(NC(=O)N(c2ccccc2C(C)C)[C@H]2CC[C@H](c3cccnc3)CC2)c(OC(F)F)n1]

In embodiments, the compound is

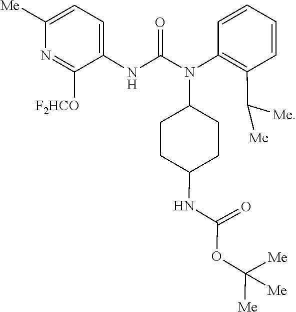 [SMILES: Cc1ccc(NC(=O)N(c2ccccc2C(C)C)C2CCC(NC(=O)OC(C)(C)C)CC2)c(OC(F)F)n1]

In embodiments, the compound is

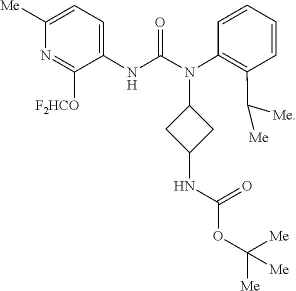 [SMILES: Cc1ccc(NC(=O)N(c2ccccc2C(C)C)C2CC(NC(=O)OC(C)(C)C)C2)c(OC(F)F)n1]

In embodiments, the compound is

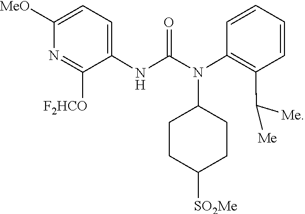 [SMILES: COc1ccc(NC(=O)N(c2ccccc2C(C)C)C2CCC(S(C)(=O)=O)CC2)c(OC(F)F)n1]

In embodiments, the compound is

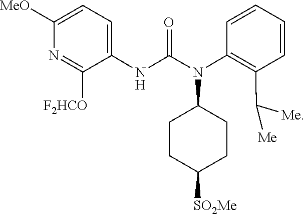 [SMILES: COc1ccc(NC(=O)N(c2ccccc2C(C)C)[C@H]2CC[C@@H](S(C)(=O)=O)CC2)c(OC(F)F)n1]

In embodiments, the compound is

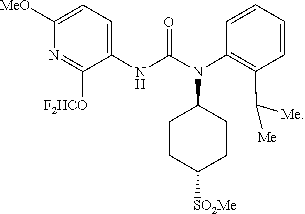 [SMILES: COc1ccc(NC(=O)N(c2ccccc2C(C)C)[C@H]2CC[C@H](S(C)(=O)=O)CC2)c(OC(F)F)n1]

In embodiments, the compound is

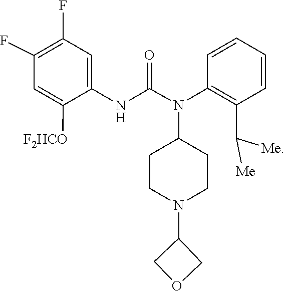 [SMILES: CC(C)c1ccccc1N(C(=O)Nc1cc(F)c(F)cc1OC(F)F)C1CCN(C2COC2)CC1]

In embodiments, the compound is

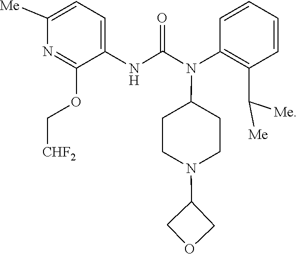 [SMILES: Cc1ccc(NC(=O)N(c2ccccc2C(C)C)C2CCN(C3COC3)CC2)c(OCC(F)F)n1]

In embodiments, the compound is

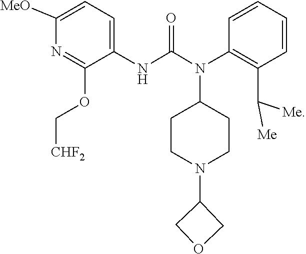 [SMILES: COc1ccc(NC(=O)N(c2ccccc2C(C)C)C2CCN(C3COC3)CC2)c(OCC(F)F)n1]

In embodiments, the compound is

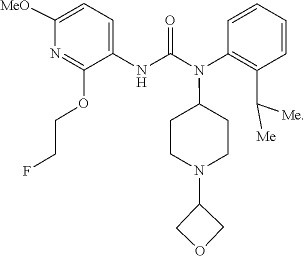 [SMILES: COc1ccc(NC(=O)N(c2ccccc2C(C)C)C2CCN(C3COC3)CC2)c(OCCF)n1]

In embodiments, the compound is

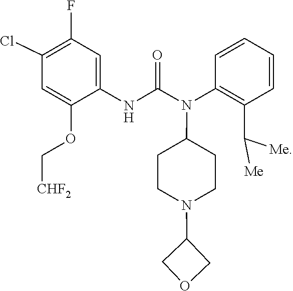 [SMILES: CC(C)c1ccccc1N(C(=O)Nc1cc(F)c(Cl)cc1OCC(F)F)C1CCN(C2COC2)CC1]

In embodiments, the compound is

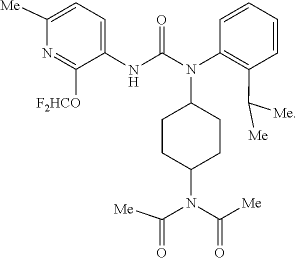 [SMILES: CC(=O)N(C(C)=O)C1CCC(N(C(=O)Nc2ccc(C)nc2OC(F)F)c2ccccc2C(C)C)CC1]

In embodiments, the compound is

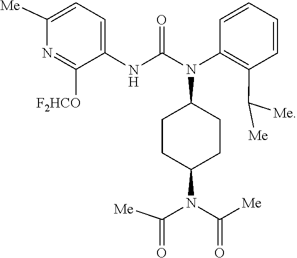 [SMILES: CC(=O)N(C(C)=O)[C@H]1CC[C@@H](N(C(=O)Nc2ccc(C)nc2OC(F)F)c2ccccc2C(C)C)CC1]

In embodiments, the compound is

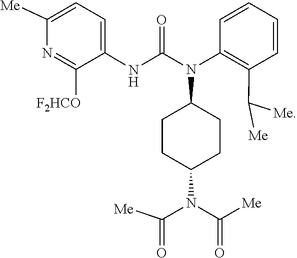 [SMILES: CC(=O)N(C(C)=O)[C@H]1CC[C@H](N(C(=O)Nc2ccc(C)nc2OC(F)F)c2ccccc2C(C)C)CC1]

In embodiments, the compound is

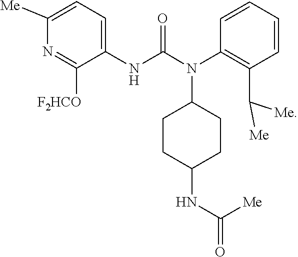 [SMILES: CC(=O)NC1CCC(N(C(=O)Nc2ccc(C)nc2OC(F)F)c2ccccc2C(C)C)CC1]

In embodiments, the compound is

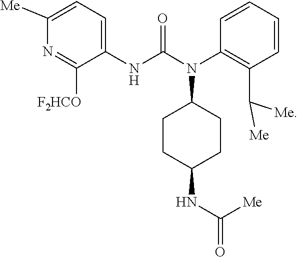 [SMILES: CC(=O)N[C@H]1CC[C@@H](N(C(=O)Nc2ccc(C)nc2OC(F)F)c2ccccc2C(C)C)CC1]

In embodiments, the compound is

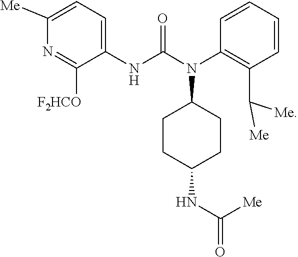 [SMILES: CC(=O)N[C@H]1CC[C@H](N(C(=O)Nc2ccc(C)nc2OC(F)F)c2ccccc2C(C)C)CC1]

In embodiments, the compound is

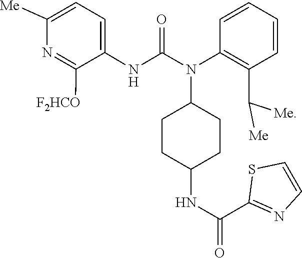 [SMILES: Cc1ccc(NC(=O)N(c2ccccc2C(C)C)C2CCC(NC(=O)c3nccs3)CC2)c(OC(F)F)n1]

In embodiments, the compound is

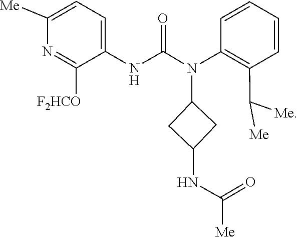 [SMILES: CC(=O)NC1CC(N(C(=O)Nc2ccc(C)nc2OC(F)F)c2ccccc2C(C)C)C1]

In embodiments, the compound is

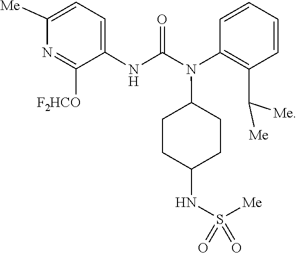 [SMILES: Cc1ccc(NC(=O)N(c2ccccc2C(C)C)C2CCC(NS(C)(=O)=O)CC2)c(OC(F)F)n1]

In embodiments, the compound is

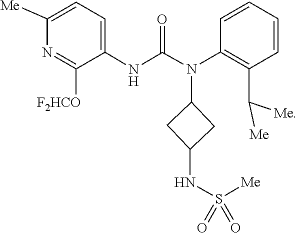 [SMILES: Cc1ccc(NC(=O)N(c2ccccc2C(C)C)C2CC(NS(C)(=O)=O)C2)c(OC(F)F)n1]

In embodiments, the compound is

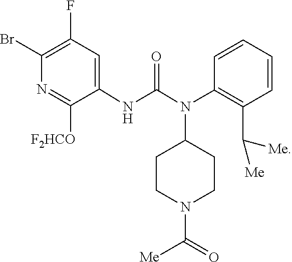 [SMILES: CC(=O)N1CCC(N(C(=O)Nc2cc(F)c(Br)nc2OC(F)F)c2ccccc2C(C)C)CC1]

In embodiments, the compound is

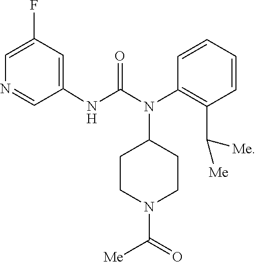 [SMILES: CC(=O)N1CCC(N(C(=O)Nc2cncc(F)c2)c2ccccc2C(C)C)CC1]

In embodiments, the compound is

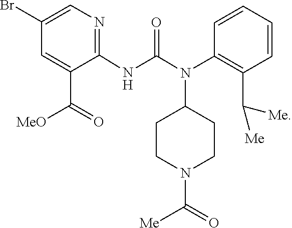 [SMILES: COC(=O)c1cc(Br)cnc1NC(=O)N(c1ccccc1C(C)C)C1CCN(C(C)=O)CC1]

In embodiments, the compound is

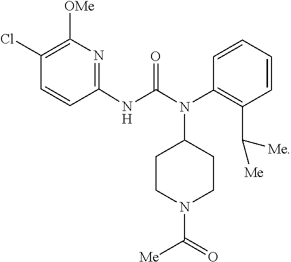 [SMILES: COc1nc(NC(=O)N(c2ccccc2C(C)C)C2CCN(C(C)=O)CC2)ccc1Cl]

In embodiments, the compound is

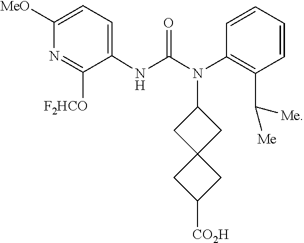 [SMILES: COc1ccc(NC(=O)N(c2ccccc2C(C)C)C2CC3(CC(C(=O)O)C3)C2)c(OC(F)F)n1]

In embodiments, the compound is

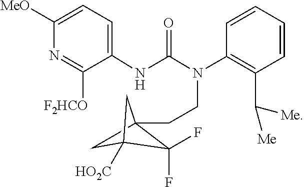 [SMILES: COc1ccc(NC(=O)N(CCC23CC(C(=O)O)(C2)C3(F)F)c2ccccc2C(C)C)c(OC(F)F)n1]

In embodiments, the compound is

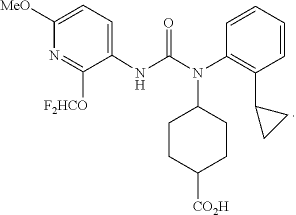 [SMILES: COc1ccc(NC(=O)N(c2ccccc2C2CC2)C2CCC(C(=O)O)CC2)c(OC(F)F)n1]

In embodiments, the compound is

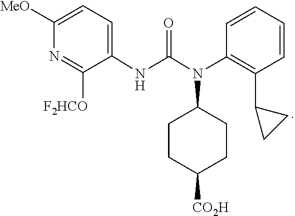 [SMILES: COc1ccc(NC(=O)N(c2ccccc2C2CC2)[C@H]2CC[C@@H](C(=O)O)CC2)c(OC(F)F)n1]

In embodiments, the compound is

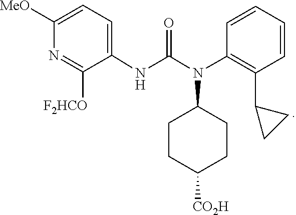 [SMILES: COc1ccc(NC(=O)N(c2ccccc2C2CC2)[C@H]2CC[C@H](C(=O)O)CC2)c(OC(F)F)n1]

In embodiments, the compound is

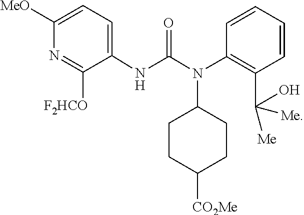 [SMILES: COC(=O)C1CCC(N(C(=O)Nc2ccc(OC)nc2OC(F)F)c2ccccc2C(C)(C)O)CC1]

In embodiments, the compound is

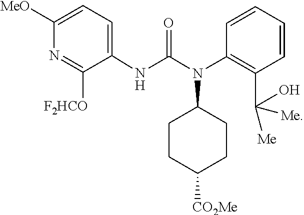 [SMILES: COc1ccc(NC(=O)N(c2ccccc2C(C)(C)O)[C@H]2CC[C@H](C(=O)OC)CC2)c(OC(F)F)n1]

In embodiments, the compound is

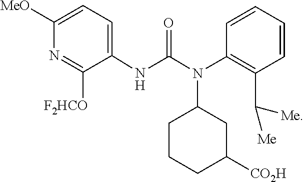 [SMILES: COc1ccc(NC(=O)N(c2ccccc2C(C)C)C2CCCC(C(=O)O)C2)c(OC(F)F)n1]

In embodiments, the compound is

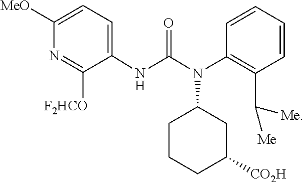 [SMILES: COc1ccc(NC(=O)N(c2ccccc2C(C)C)[C@H]2CCC[C@@H](C(=O)O)C2)c(OC(F)F)n1]

In embodiments, the compound is

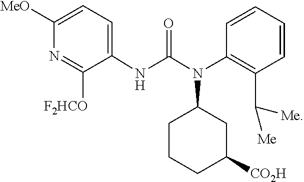 [SMILES: COc1ccc(NC(=O)N(c2ccccc2C(C)C)[C@@H]2CCC[C@H](C(=O)O)C2)c(OC(F)F)n1]

In embodiments, the compound is

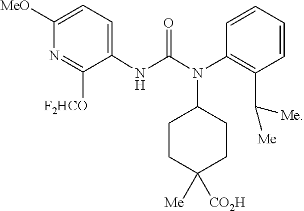 [SMILES: COc1ccc(NC(=O)N(c2ccccc2C(C)C)C2CCC(C)(C(=O)O)CC2)c(OC(F)F)n1]

In embodiments, the compound is

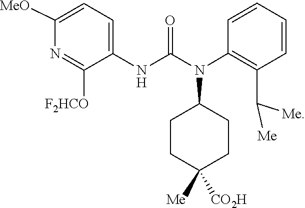 [SMILES: COc1ccc(NC(=O)N(c2ccccc2C(C)C)[C@H]2CC[C@](C)(C(=O)O)CC2)c(OC(F)F)n1]

In embodiments, the compound is

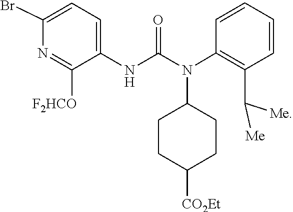 [SMILES: CCOC(=O)C1CCC(N(C(=O)Nc2ccc(Br)nc2OC(F)F)c2ccccc2C(C)C)CC1]

In embodiments, the compound is

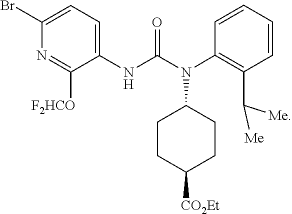 [SMILES: CCOC(=O)[C@H]1CC[C@H](N(C(=O)Nc2ccc(Br)nc2OC(F)F)c2ccccc2C(C)C)CC1]

In embodiments, the compound is

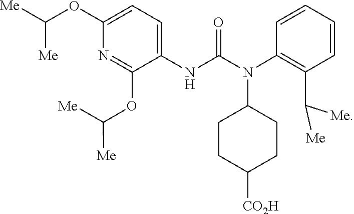 [SMILES: CC(C)Oc1ccc(NC(=O)N(c2ccccc2C(C)C)C2CCC(C(=O)O)CC2)c(OC(C)C)n1]

In embodiments, the compound is

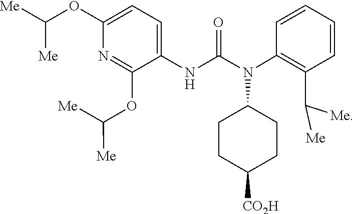 [SMILES: CC(C)Oc1ccc(NC(=O)N(c2ccccc2C(C)C)[C@H]2CC[C@H](C(=O)O)CC2)c(OC(C)C)n1]

In embodiments, the compound is

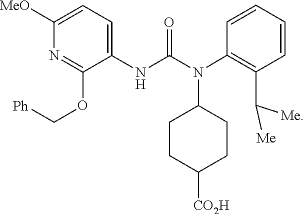 [SMILES: COc1ccc(NC(=O)N(c2ccccc2C(C)C)C2CCC(C(=O)O)CC2)c(OCc2ccccc2)n1]

In embodiments, the compound is

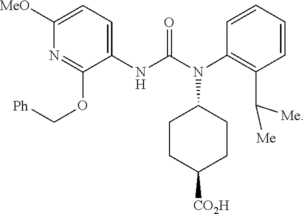 [SMILES: COc1ccc(NC(=O)N(c2ccccc2C(C)C)[C@H]2CC[C@H](C(=O)O)CC2)c(OCc2ccccc2)n1]

In embodiments, the compound is

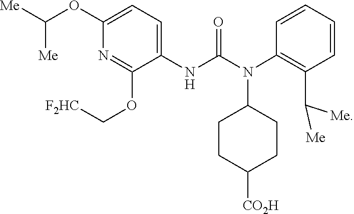 [SMILES: CC(C)Oc1ccc(NC(=O)N(c2ccccc2C(C)C)C2CCC(C(=O)O)CC2)c(OCC(F)F)n1]

In embodiments, the compound is

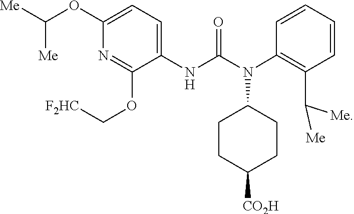 [SMILES: CC(C)Oc1ccc(NC(=O)N(c2ccccc2C(C)C)[C@H]2CC[C@H](C(=O)O)CC2)c(OCC(F)F)n1]

In embodiments, the compound is

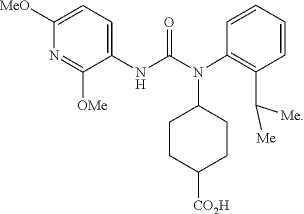 [SMILES: COc1ccc(NC(=O)N(c2ccccc2C(C)C)C2CCC(C(=O)O)CC2)c(OC)n1]

In embodiments, the compound is

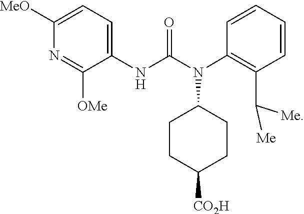 [SMILES: COc1ccc(NC(=O)N(c2ccccc2C(C)C)[C@H]2CC[C@H](C(=O)O)CC2)c(OC)n1]

In embodiments, the compound is

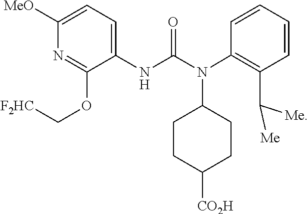 [SMILES: COc1ccc(NC(=O)N(c2ccccc2C(C)C)C2CCC(C(=O)O)CC2)c(OCC(F)F)n1]

In embodiments, the compound is

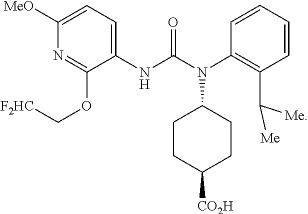 [SMILES: COc1ccc(NC(=O)N(c2ccccc2C(C)C)[C@H]2CC[C@H](C(=O)O)CC2)c(OCC(F)F)n1]

In embodiments, the compound is

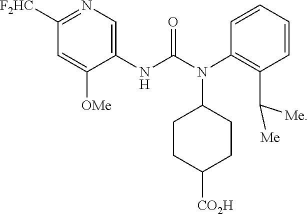 [SMILES: COc1cc(C(F)F)ncc1NC(=O)N(c1ccccc1C(C)C)C1CCC(C(=O)O)CC1]

In embodiments, the compound is

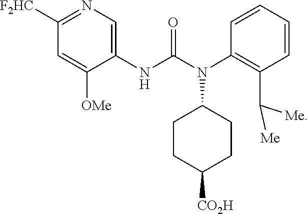 [SMILES: COc1cc(C(F)F)ncc1NC(=O)N(c1ccccc1C(C)C)[C@H]1CC[C@H](C(=O)O)CC1]

In embodiments, the compound is

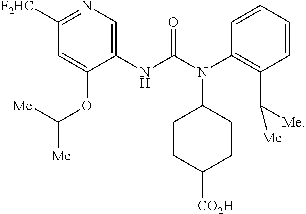 [SMILES: CC(C)Oc1cc(C(F)F)ncc1NC(=O)N(c1ccccc1C(C)C)C1CCC(C(=O)O)CC1]

In embodiments, the compound is

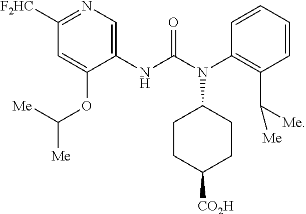 [SMILES: CC(C)Oc1cc(C(F)F)ncc1NC(=O)N(c1ccccc1C(C)C)[C@H]1CC[C@H](C(=O)O)CC1]

In embodiments, the compound is

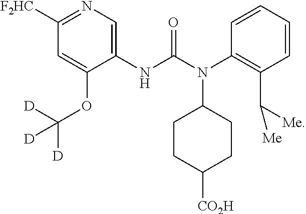 [SMILES: [2H]C([2H])([2H])Oc1cc(C(F)F)ncc1NC(=O)N(c1ccccc1C(C)C)C1CCC(C(=O)O)CC1]

In embodiments, the compound is

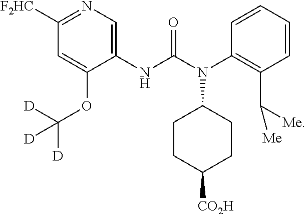 [SMILES: [2H]C([2H])([2H])Oc1cc(C(F)F)ncc1NC(=O)N(c1ccccc1C(C)C)[C@H]1CC[C@H](C(=O)O)CC1]

In embodiments, the compound is

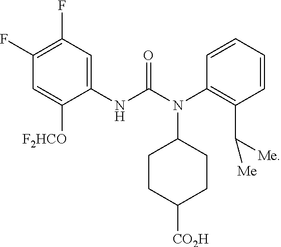 [SMILES: CC(C)c1ccccc1N(C(=O)Nc1cc(F)c(F)cc1OC(F)F)C1CCC(C(=O)O)CC1]

In embodiments, the compound is

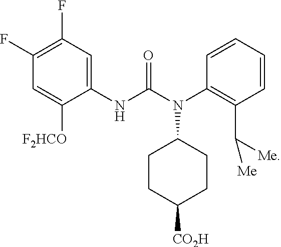 [SMILES: CC(C)c1ccccc1N(C(=O)Nc1cc(F)c(F)cc1OC(F)F)[C@H]1CC[C@H](C(=O)O)CC1]

In embodiments, the compound is

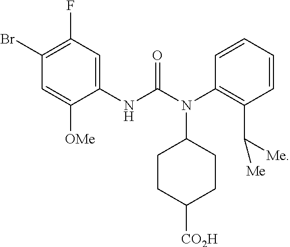 [SMILES: COc1cc(Br)c(F)cc1NC(=O)N(c1ccccc1C(C)C)C1CCC(C(=O)O)CC1]

In embodiments, the compound is

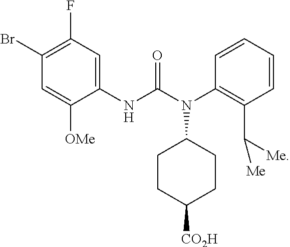 [SMILES: COc1cc(Br)c(F)cc1NC(=O)N(c1ccccc1C(C)C)[C@H]1CC[C@H](C(=O)O)CC1]

In embodiments, the compound is

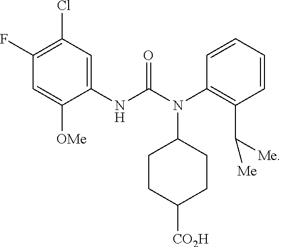 [SMILES: COc1cc(F)c(Cl)cc1NC(=O)N(c1ccccc1C(C)C)C1CCC(C(=O)O)CC1]

In embodiments, the compound is

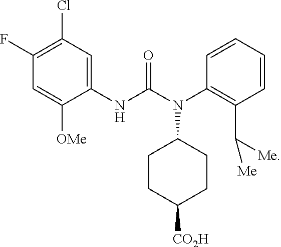 [SMILES: COc1cc(F)c(Cl)cc1NC(=O)N(c1ccccc1C(C)C)[C@H]1CC[C@H](C(=O)O)CC1]

In embodiments, the compound is

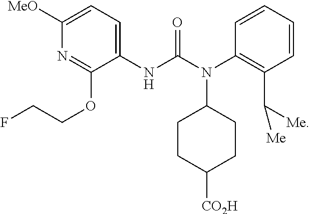 [SMILES: COc1ccc(NC(=O)N(c2ccccc2C(C)C)C2CCC(C(=O)O)CC2)c(OCCF)n1]

In embodiments, the compound is

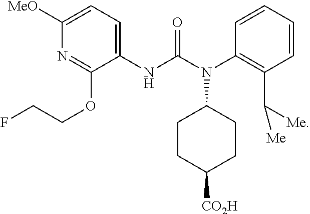 [SMILES: COc1ccc(NC(=O)N(c2ccccc2C(C)C)[C@H]2CC[C@H](C(=O)O)CC2)c(OCCF)n1]

In embodiments, the compound is

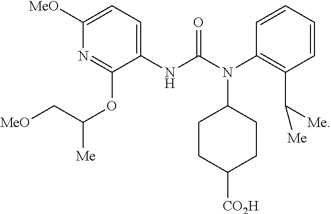 [SMILES: COCC(C)Oc1nc(OC)ccc1NC(=O)N(c1ccccc1C(C)C)C1CCC(C(=O)O)CC1]

In embodiments, the compound is

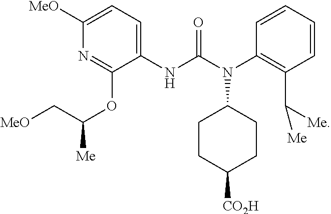 [SMILES: COC[C@H](C)Oc1nc(OC)ccc1NC(=O)N(c1ccccc1C(C)C)[C@H]1CC[C@H](C(=O)O)CC1]

In embodiments, the compound is

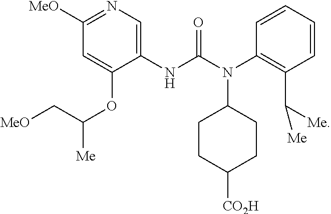 [SMILES: COCC(C)Oc1cc(OC)ncc1NC(=O)N(c1ccccc1C(C)C)C1CCC(C(=O)O)CC1]

In embodiments, the compound is

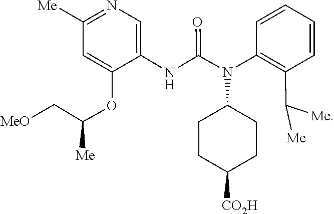 [SMILES: COC[C@H](C)Oc1cc(C)ncc1NC(=O)N(c1ccccc1C(C)C)[C@H]1CC[C@H](C(=O)O)CC1]

In embodiments, the compound is

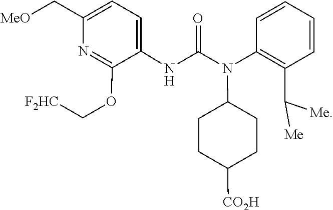 [SMILES: COCc1ccc(NC(=O)N(c2ccccc2C(C)C)C2CCC(C(=O)O)CC2)c(OCC(F)F)n1]

In embodiments, the compound is

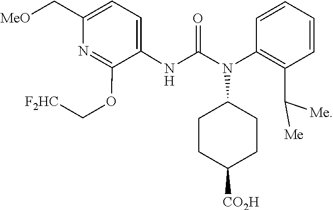

In embodiments, the compound is

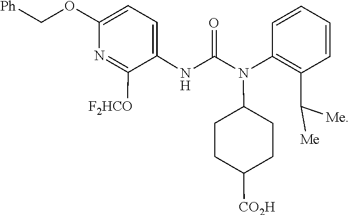 [SMILES: CC(C)c1ccccc1N(C(=O)Nc1ccc(OCc2ccccc2)nc1OC(F)F)C1CCC(C(=O)O)CC1]

In embodiments, the compound is

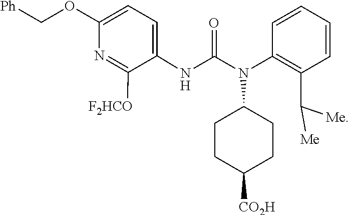

In embodiments, the compound is

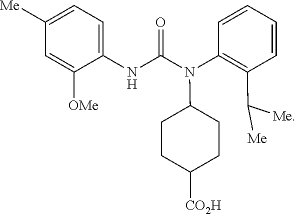 [SMILES: COc1cc(C)ccc1NC(=O)N(c1ccccc1C(C)C)C1CCC(C(=O)O)CC1]

In embodiments, the compound is

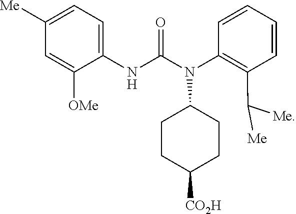

In embodiments, the compound is

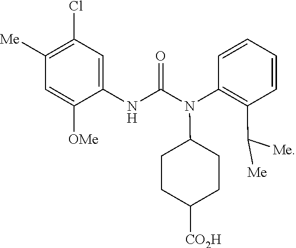 [SMILES: COc1cc(C)c(Cl)cc1NC(=O)N(c1ccccc1C(C)C)C1CCC(C(=O)O)CC1]

In embodiments, the compound is

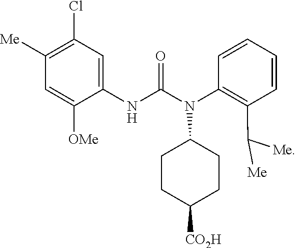

In embodiments, the compound is

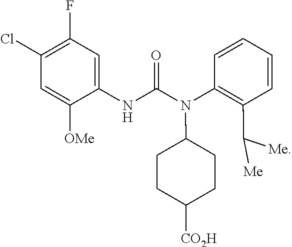 [SMILES: COc1cc(Cl)c(F)cc1NC(=O)N(c1ccccc1C(C)C)C1CCC(C(=O)O)CC1]

In embodiments, the compound is

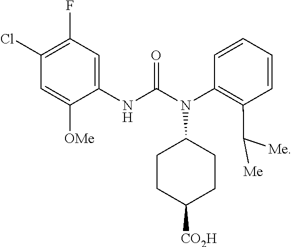

In embodiments, the compound is

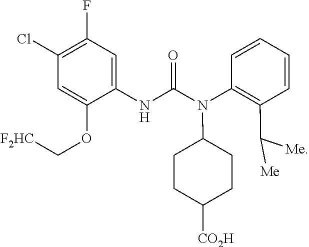 [SMILES: CC(C)c1ccccc1N(C(=O)Nc1cc(F)c(Cl)cc1OCC(F)F)C1CCC(C(=O)O)CC1]

In embodiments, the compound is

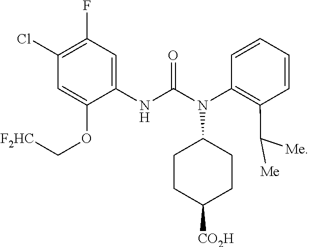

In embodiments, the compound is

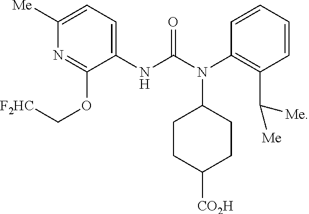 [SMILES: Cc1ccc(NC(=O)N(c2ccccc2C(C)C)C2CCC(C(=O)O)CC2)c(OCC(F)F)n1]

In embodiments, the compound is

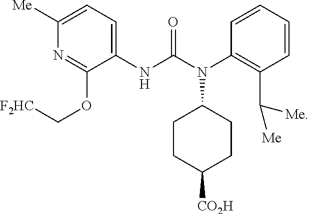

In embodiments, the compound is

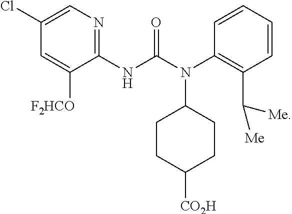 [SMILES: CC(C)c1ccccc1N(C(=O)Nc1ncc(Cl)cc1OC(F)F)C1CCC(C(=O)O)CC1]

In embodiments, the compound is

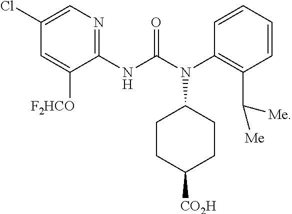 [SMILES: CC(C)c1ccccc1N(C(=O)Nc1ncc(Cl)cc1OC(F)F)[C@H]1CC[C@H](C(=O)O)CC1]

In embodiments, the compound is

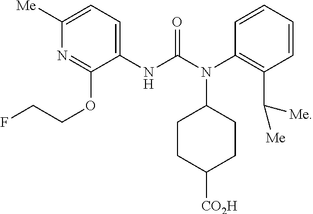 [SMILES: Cc1ccc(NC(=O)N(c2ccccc2C(C)C)C2CCC(C(=O)O)CC2)c(OCCF)n1]

In embodiments, the compound is

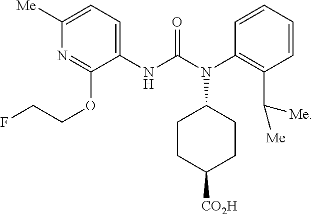 [SMILES: Cc1ccc(NC(=O)N(c2ccccc2C(C)C)[C@H]2CC[C@H](C(=O)O)CC2)c(OCCF)n1]

In embodiments, the compound is

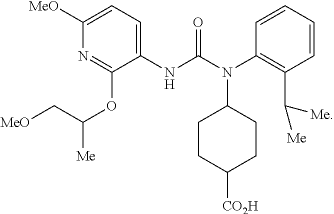 [SMILES: COCC(C)Oc1nc(OC)ccc1NC(=O)N(c1ccccc1C(C)C)C1CCC(C(=O)O)CC1]

In embodiments, the compound is

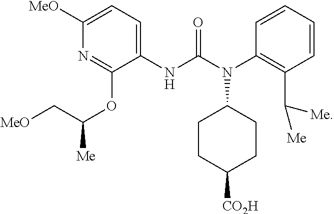 [SMILES: COC[C@H](C)Oc1nc(OC)ccc1NC(=O)N(c1ccccc1C(C)C)[C@H]1CC[C@H](C(=O)O)CC1]

In embodiments, the compound is

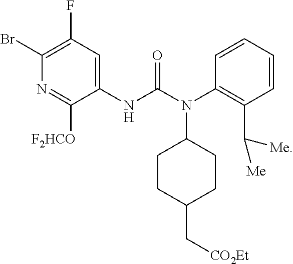 [SMILES: CCOC(=O)CC1CCC(N(C(=O)Nc2cc(F)c(Br)nc2OC(F)F)c2ccccc2C(C)C)CC1]

In embodiments, the compound is

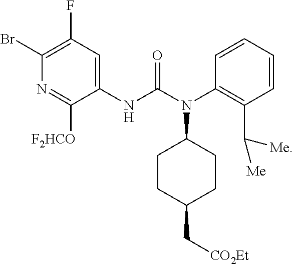 [SMILES: CCOC(=O)C[C@H]1CC[C@@H](N(C(=O)Nc2cc(F)c(Br)nc2OC(F)F)c2ccccc2C(C)C)CC1]

In embodiments, the compound is

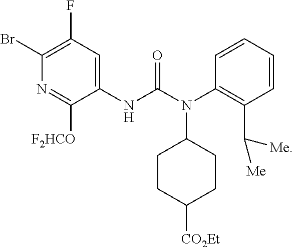 [SMILES: CCOC(=O)C1CCC(N(C(=O)Nc2cc(F)c(Br)nc2OC(F)F)c2ccccc2C(C)C)CC1]

In embodiments, the compound is

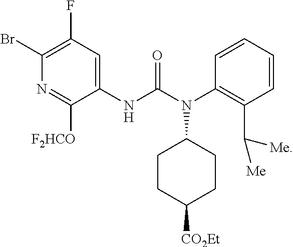 [SMILES: CCOC(=O)[C@H]1CC[C@H](N(C(=O)Nc2cc(F)c(Br)nc2OC(F)F)c2ccccc2C(C)C)CC1]

In embodiments, the compound is

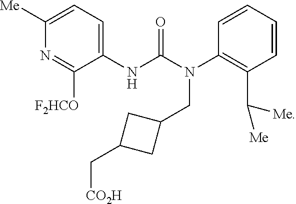 [SMILES: Cc1ccc(NC(=O)N(CC2CC(CC(=O)O)C2)c2ccccc2C(C)C)c(OC(F)F)n1]

In embodiments, the compound is

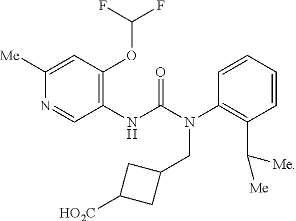 [SMILES: Cc1cc(OC(F)F)c(NC(=O)N(CC2CC(C(=O)O)C2)c2ccccc2C(C)C)cn1]

In embodiments, the compound is

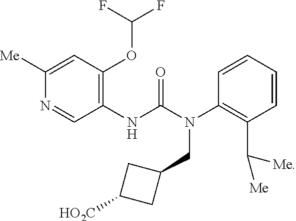 [SMILES: Cc1cc(OC(F)F)c(NC(=O)N(C[C@H]2C[C@H](C(=O)O)C2)c2ccccc2C(C)C)cn1]

In embodiments, the compound is

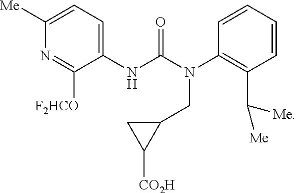 [SMILES: Cc1ccc(NC(=O)N(CC2CC2C(=O)O)c2ccccc2C(C)C)c(OC(F)F)n1]

In embodiments, the compound is

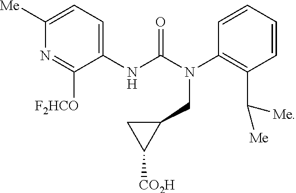 [SMILES: Cc1ccc(NC(=O)N(C[C@@H]2C[C@H]2C(=O)O)c2ccccc2C(C)C)c(OC(F)F)n1]

In embodiments, the compound is

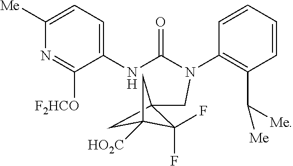 [SMILES: Cc1ccc(NC(=O)N(CC23CC(C(=O)O)(C2)C3(F)F)c2ccccc2C(C)C)c(OC(F)F)n1]

In embodiments, the compound is

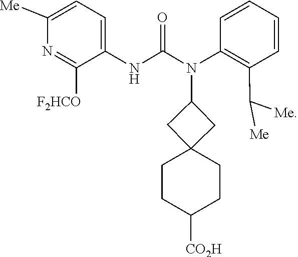 [SMILES: Cc1ccc(NC(=O)N(c2ccccc2C(C)C)C2CC3(CCC(C(=O)O)CC3)C2)c(OC(F)F)n1]

In embodiments, the compound is

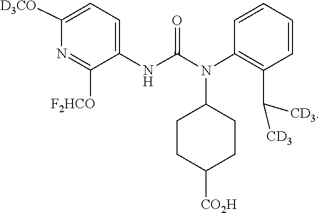

In embodiments, the compound is

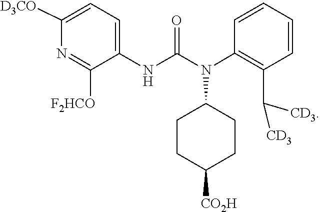

In embodiments, the compound is

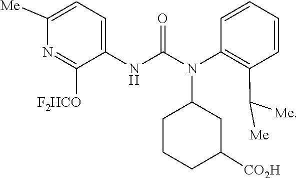 [SMILES: Cc1ccc(NC(=O)N(c2ccccc2C(C)C)C2CCCC(C(=O)O)C2)c(OC(F)F)n1]

In embodiments, the compound is

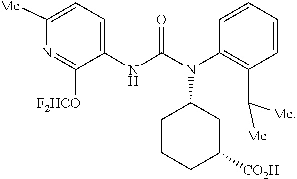 [SMILES: Cc1ccc(NC(=O)N(c2ccccc2C(C)C)[C@H]2CCC[C@@H](C(=O)O)C2)c(OC(F)F)n1]

In embodiments, the compound is

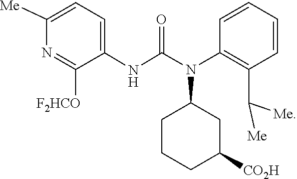 [SMILES: Cc1ccc(NC(=O)N(c2ccccc2C(C)C)[C@@H]2CCC[C@H](C(=O)O)C2)c(OC(F)F)n1]

In embodiments, the compound is

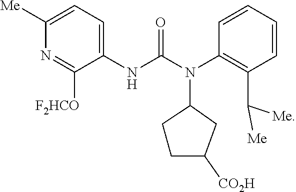 [SMILES: Cc1ccc(NC(=O)N(c2ccccc2C(C)C)C2CCC(C(=O)O)C2)c(OC(F)F)n1]

In embodiments, the compound is

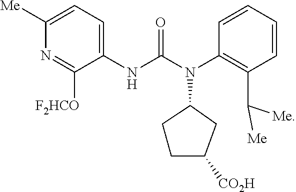 [SMILES: Cc1ccc(NC(=O)N(c2ccccc2C(C)C)[C@H]2CC[C@@H](C(=O)O)C2)c(OC(F)F)n1]

In embodiments, the compound is

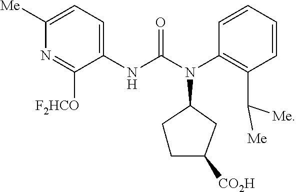 [SMILES: Cc1ccc(NC(=O)N(c2ccccc2C(C)C)[C@@H]2CC[C@H](C(=O)O)C2)c(OC(F)F)n1]

In embodiments, the compound is

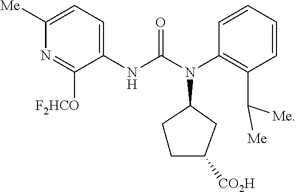 [SMILES: Cc1ccc(NC(=O)N(c2ccccc2C(C)C)[C@@H]2CC[C@@H](C(=O)O)C2)c(OC(F)F)n1]

In embodiments, the compound is

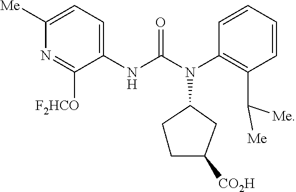 [SMILES: Cc1ccc(NC(=O)N(c2ccccc2C(C)C)[C@H]2CC[C@H](C(=O)O)C2)c(OC(F)F)n1]

In embodiments, the compound is

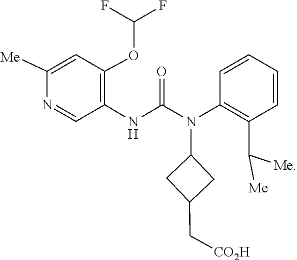 [SMILES: Cc1cc(OC(F)F)c(NC(=O)N(c2ccccc2C(C)C)C2CC(CC(=O)O)C2)cn1]

In embodiments, the compound is

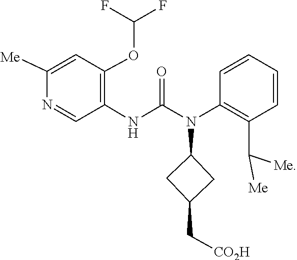 [SMILES: Cc1cc(OC(F)F)c(NC(=O)N(c2ccccc2C(C)C)[C@H]2C[C@@H](CC(=O)O)C2)cn1]

In embodiments, the compound is

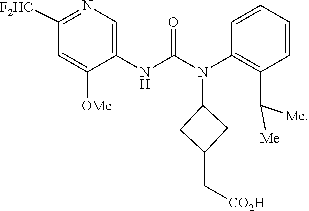 [SMILES: COc1cc(C(F)F)ncc1NC(=O)N(c1ccccc1C(C)C)C1CC(CC(=O)O)C1]

In embodiments, the compound is

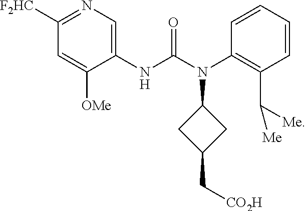 [SMILES: COc1cc(C(F)F)ncc1NC(=O)N(c1ccccc1C(C)C)[C@H]1C[C@@H](CC(=O)O)C1]

In embodiments, the compound is

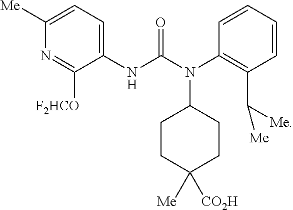 [SMILES: Cc1ccc(NC(=O)N(c2ccccc2C(C)C)C2CCC(C)(C(=O)O)CC2)c(OC(F)F)n1]

In embodiments, the compound is

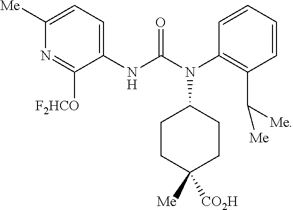 [SMILES: Cc1ccc(NC(=O)N(c2ccccc2C(C)C)[C@H]2CC[C@@](C)(C(=O)O)CC2)c(OC(F)F)n1]

In embodiments, the compound is

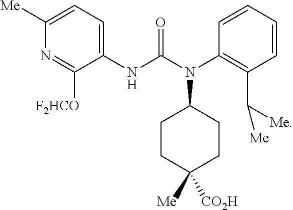 [SMILES: Cc1ccc(NC(=O)N(c2ccccc2C(C)C)[C@H]2CC[C@](C)(C(=O)O)CC2)c(OC(F)F)n1]

In embodiments, the compound is

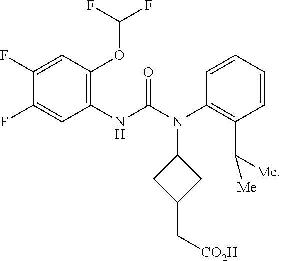 [SMILES: CC(C)c1ccccc1N(C(=O)Nc1cc(F)c(F)cc1OC(F)F)C1CC(CC(=O)O)C1]

In embodiments, the compound is

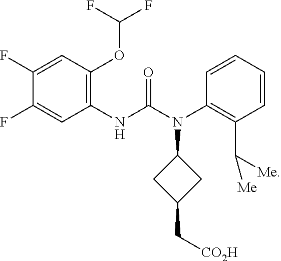 [SMILES: CC(C)c1ccccc1N(C(=O)Nc1cc(F)c(F)cc1OC(F)F)[C@H]1C[C@@H](CC(=O)O)C1]

In embodiments, the compound is

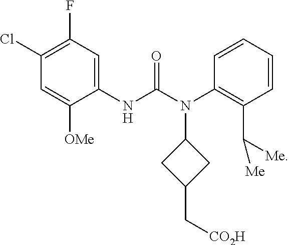 [SMILES: COc1cc(Cl)c(F)cc1NC(=O)N(c1ccccc1C(C)C)C1CC(CC(=O)O)C1]

In embodiments, the compound is

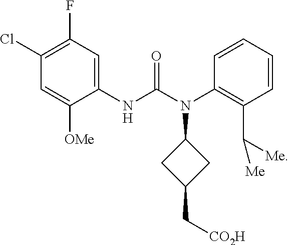 [SMILES: COc1cc(Cl)c(F)cc1NC(=O)N(c1ccccc1C(C)C)[C@H]1C[C@@H](CC(=O)O)C1]

In embodiments, the compound is

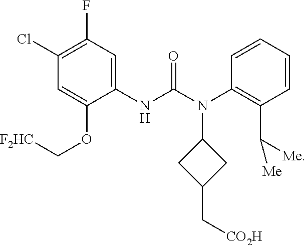 [SMILES: CC(C)c1ccccc1N(C(=O)Nc1cc(F)c(Cl)cc1OCC(F)F)C1CC(CC(=O)O)C1]

In embodiments, the compound is

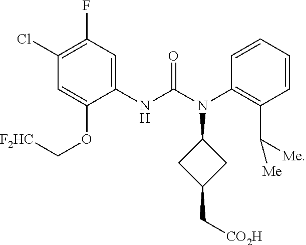 [SMILES: CC(C)c1ccccc1N(C(=O)Nc1cc(F)c(Cl)cc1OCC(F)F)[C@H]1C[C@@H](CC(=O)O)C1]

In embodiments, the compound is

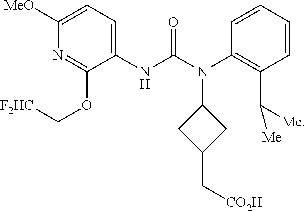 [SMILES: COc1ccc(NC(=O)N(c2ccccc2C(C)C)C2CC(CC(=O)O)C2)c(OCC(F)F)n1]

In embodiments, the compound is

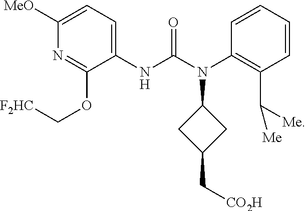 [SMILES: COc1ccc(NC(=O)N(c2ccccc2C(C)C)[C@H]2C[C@@H](CC(=O)O)C2)c(OCC(F)F)n1]

In embodiments, the compound is

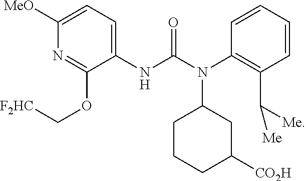 [SMILES: COc1ccc(NC(=O)N(c2ccccc2C(C)C)C2CCCC(C(=O)O)C2)c(OCC(F)F)n1]

In embodiments, the compound is

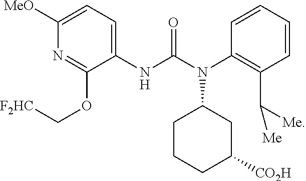 [SMILES: COc1ccc(NC(=O)N(c2ccccc2C(C)C)[C@H]2CCC[C@@H](C(=O)O)C2)c(OCC(F)F)n1]

In embodiments, the compound is

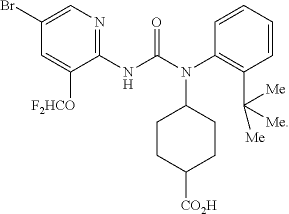 [SMILES: CC(C)(C)c1ccccc1N(C(=O)Nc1ncc(Br)cc1OC(F)F)C1CCC(C(=O)O)CC1]

In embodiments, the compound is

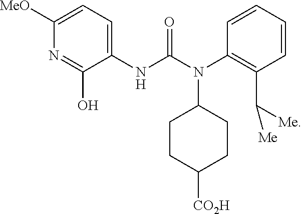 [SMILES: COc1ccc(NC(=O)N(c2ccccc2C(C)C)C2CCC(C(=O)O)CC2)c(O)n1]

In embodiments, the compound is

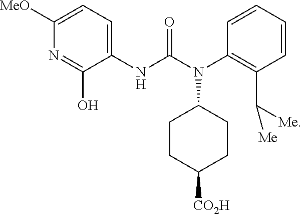 [SMILES: COc1ccc(NC(=O)N(c2ccccc2C(C)C)[C@H]2CC[C@H](C(=O)O)CC2)c(O)n1]

In embodiments, the compound is

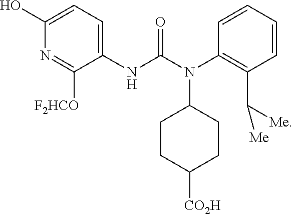 [SMILES: CC(C)c1ccccc1N(C(=O)Nc1ccc(O)nc1OC(F)F)C1CCC(C(=O)O)CC1]

In embodiments, the compound is

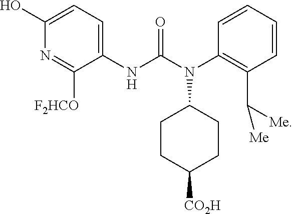 [SMILES: CC(C)c1ccccc1N(C(=O)Nc1ccc(O)nc1OC(F)F)[C@H]1CC[C@H](C(=O)O)CC1]

In embodiments, the compound is

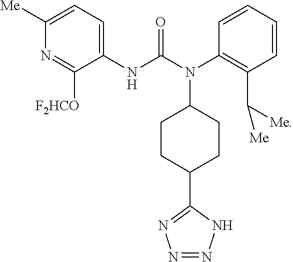 [SMILES: Cc1ccc(NC(=O)N(c2ccccc2C(C)C)C2CCC(c3nnn[nH]3)CC2)c(OC(F)F)n1]

In embodiments, the compound is

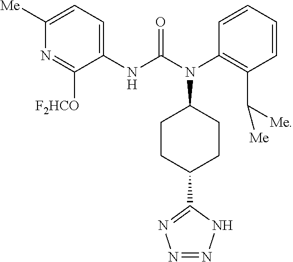 [SMILES: Cc1ccc(NC(=O)N(c2ccccc2C(C)C)[C@H]2CC[C@H](c3nnn[nH]3)CC2)c(OC(F)F)n1]

In embodiments, the compound is

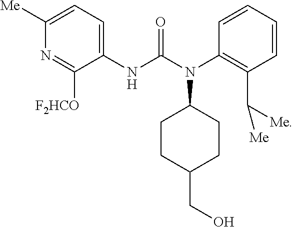

In embodiments, the compound is

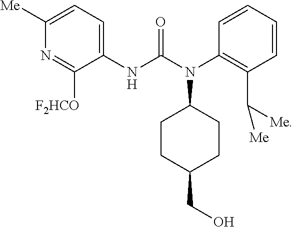 [SMILES: Cc1ccc(NC(=O)N(c2ccccc2C(C)C)[C@H]2CC[C@@H](CO)CC2)c(OC(F)F)n1]

In embodiments, the compound is

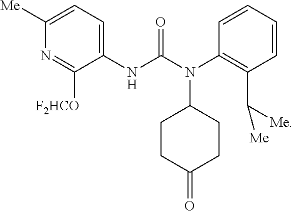 [SMILES: Cc1ccc(NC(=O)N(c2ccccc2C(C)C)C2CCC(=O)CC2)c(OC(F)F)n1]

In embodiments, the compound is

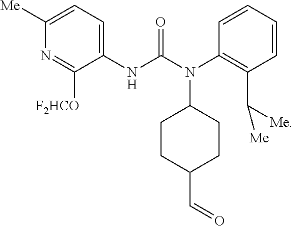 [SMILES: Cc1ccc(NC(=O)N(c2ccccc2C(C)C)C2CCC(C=O)CC2)c(OC(F)F)n1]

In embodiments, the compound is

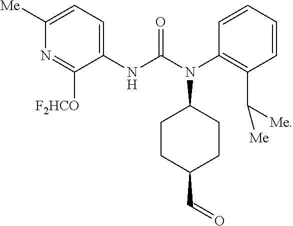 [SMILES: Cc1ccc(NC(=O)N(c2ccccc2C(C)C)[C@H]2CC[C@@H](C=O)CC2)c(OC(F)F)n1]

In embodiments, the compound is

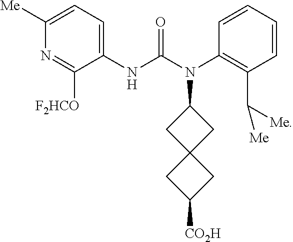 [SMILES: Cc1ccc(NC(=O)N(c2ccccc2C(C)C)[C@H]2CC3(C[C@H](C(=O)O)C3)C2)c(OC(F)F)n1]

In embodiments, the compound is

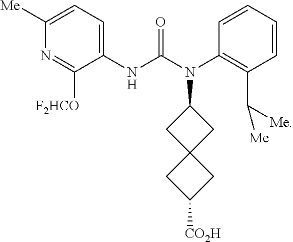 [SMILES: Cc1ccc(NC(=O)N(c2ccccc2C(C)C)[C@H]2CC3(C[C@H](C(=O)O)C3)C2)c(OC(F)F)n1]

In embodiments, the compound is

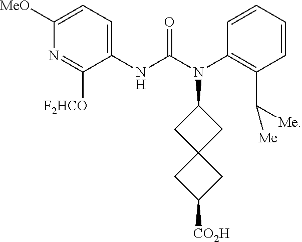 [SMILES: COc1ccc(NC(=O)N(c2ccccc2C(C)C)[C@H]2CC3(C[C@H](C(=O)O)C3)C2)c(OC(F)F)n1]

In embodiments, the compound is

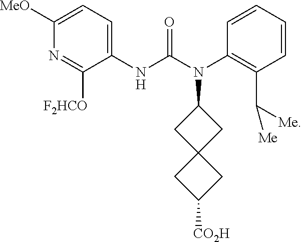 [SMILES: COc1ccc(NC(=O)N(c2ccccc2C(C)C)[C@H]2CC3(C[C@H](C(=O)O)C3)C2)c(OC(F)F)n1]

In embodiments, the compound is

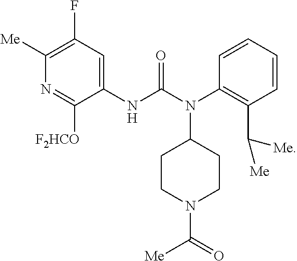 [SMILES: CC(=O)N1CCC(N(C(=O)Nc2cc(F)c(C)nc2OC(F)F)c2ccccc2C(C)C)CC1]

In embodiments, the compound is

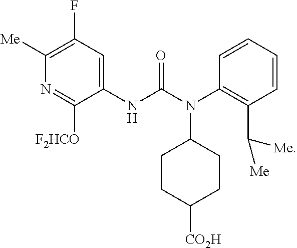 [SMILES: Cc1nc(OC(F)F)c(NC(=O)N(c2ccccc2C(C)C)C2CCC(C(=O)O)CC2)cc1F]

In embodiments, the compound is

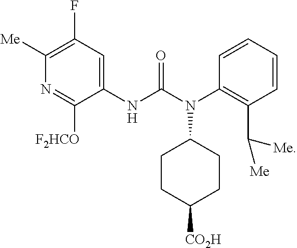 [SMILES: Cc1nc(OC(F)F)c(NC(=O)N(c2ccccc2C(C)C)[C@H]2CC[C@H](C(=O)O)CC2)cc1F]

In embodiments, the compound is

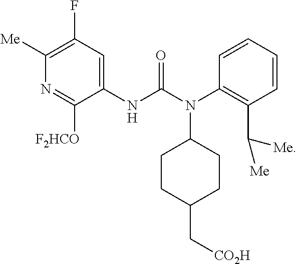 [SMILES: Cc1nc(OC(F)F)c(NC(=O)N(c2ccccc2C(C)C)C2CCC(CC(=O)O)CC2)cc1F]

In embodiments, the compound is

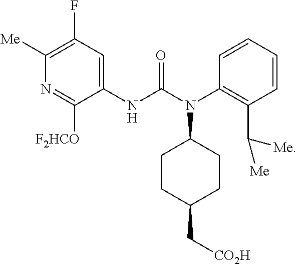 [SMILES: Cc1nc(OC(F)F)c(NC(=O)N(c2ccccc2C(C)C)[C@H]2CC[C@@H](CC(=O)O)CC2)cc1F]

In embodiments, the compound is

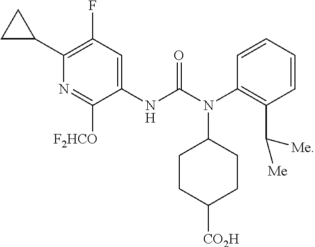 [SMILES: CC(C)c1ccccc1N(C(=O)Nc1cc(F)c(C2CC2)nc1OC(F)F)C1CCC(C(=O)O)CC1]

In embodiments, the compound is

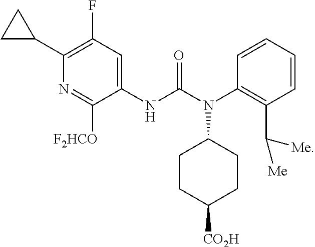 [SMILES: CC(C)c1ccccc1N(C(=O)Nc1cc(F)c(C2CC2)nc1OC(F)F)[C@H]1CC[C@H](C(=O)O)CC1]

In embodiments, the compound is

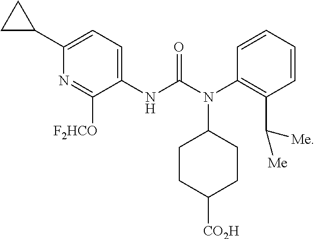 [SMILES: CC(C)c1ccccc1N(C(=O)Nc1ccc(C2CC2)nc1OC(F)F)C1CCC(C(=O)O)CC1]

In embodiments, the compound is

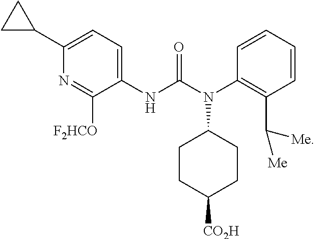 [SMILES: CC(C)c1ccccc1N(C(=O)Nc1ccc(C2CC2)nc1OC(F)F)[C@H]1CC[C@H](C(=O)O)CC1]

In embodiments, the compound is

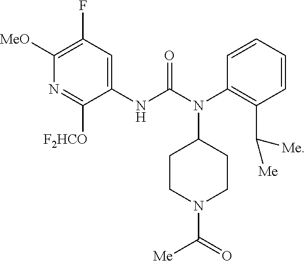 [SMILES: COc1nc(OC(F)F)c(NC(=O)N(c2ccccc2C(C)C)C2CCN(C(C)=O)CC2)cc1F]

In embodiments, the compound is

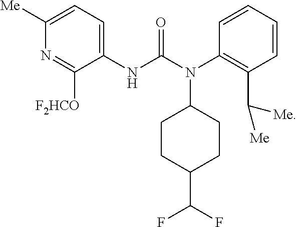 [SMILES: Cc1ccc(NC(=O)N(c2ccccc2C(C)C)C2CCC(C(F)F)CC2)c(OC(F)F)n1]

In embodiments, the compound is

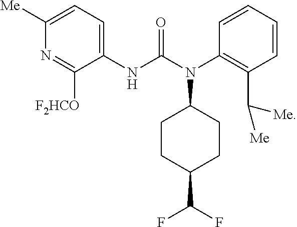 [SMILES: Cc1ccc(NC(=O)N(c2ccccc2C(C)C)[C@H]2CC[C@@H](C(F)F)CC2)c(OC(F)F)n1]

In embodiments, the compound is

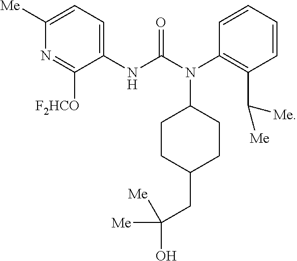 [SMILES: Cc1ccc(NC(=O)N(c2ccccc2C(C)C)C2CCC(CC(C)(C)O)CC2)c(OC(F)F)n1]

In embodiments, the compound is

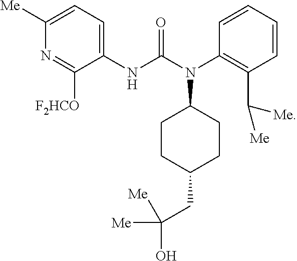 [SMILES: Cc1ccc(NC(=O)N(c2ccccc2C(C)C)[C@H]2CC[C@H](CC(C)(C)O)CC2)c(OC(F)F)n1]

In embodiments, the compound is

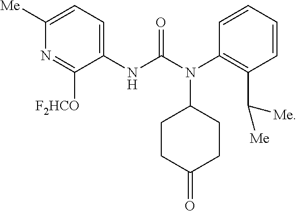 [SMILES: Cc1ccc(NC(=O)N(c2ccccc2C(C)C)C2CCC(=O)CC2)c(OC(F)F)n1]

In embodiments, the compound is

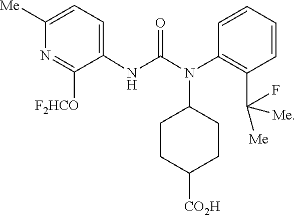 [SMILES: Cc1ccc(NC(=O)N(c2ccccc2C(C)(C)F)C2CCC(C(=O)O)CC2)c(OC(F)F)n1]

In embodiments, the compound is

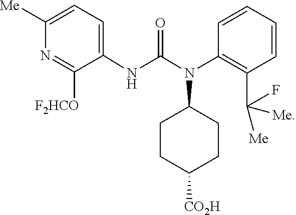 [SMILES: Cc1ccc(NC(=O)N(c2ccccc2C(C)(C)F)[C@H]2CC[C@H](C(=O)O)CC2)c(OC(F)F)n1]

In embodiments, the compound is

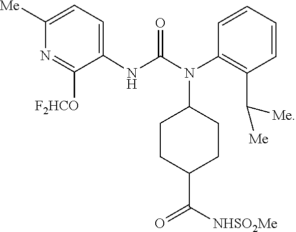

In embodiments, the compound is

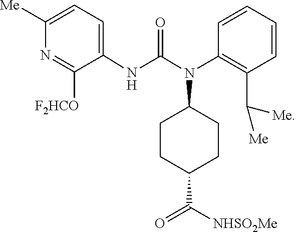

In embodiments, the compound is

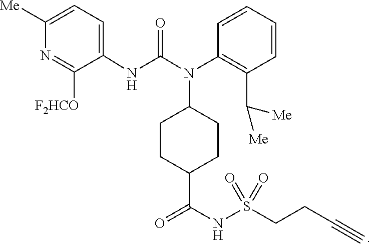 [SMILES: C#CCCS(=O)(=O)NC(=O)C1CCC(N(C(=O)Nc2ccc(C)nc2OC(F)F)c2ccccc2C(C)C)CC1]

In embodiments, the compound is

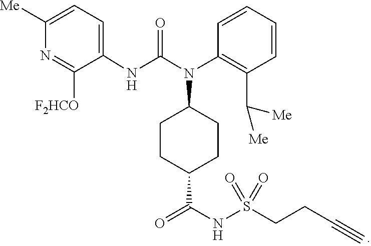 [SMILES: C#CCCS(=O)(=O)NC(=O)[C@H]1CC[C@H](N(C(=O)Nc2ccc(C)nc2OC(F)F)c2ccccc2C(C)C)CC1]

In embodiments, the compound is

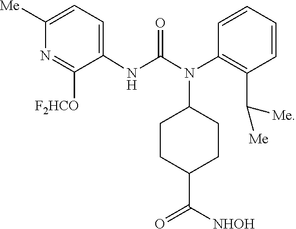

In embodiments, the compound is

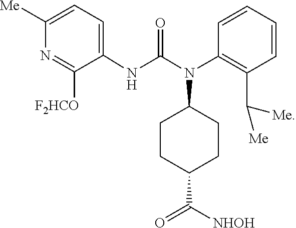

In embodiments, the compound is

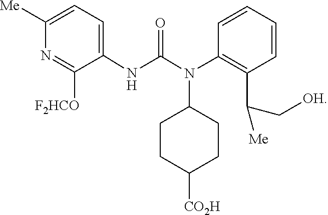 [SMILES: Cc1ccc(NC(=O)N(c2ccccc2C(C)CO)C2CCC(C(=O)O)CC2)c(OC(F)F)n1]

In embodiments, the compound is

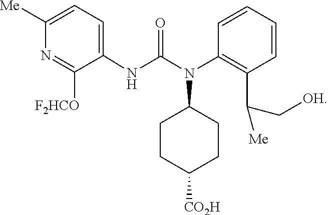 [SMILES: Cc1ccc(NC(=O)N(c2ccccc2C(C)CO)[C@H]2CC[C@H](C(=O)O)CC2)c(OC(F)F)n1]

In embodiments, the compound is

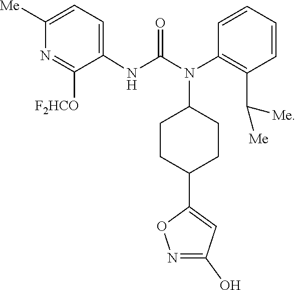 [SMILES: Cc1ccc(NC(=O)N(c2ccccc2C(C)C)C2CCC(c3cc(O)no3)CC2)c(OC(F)F)n1]

In embodiments, the compound is

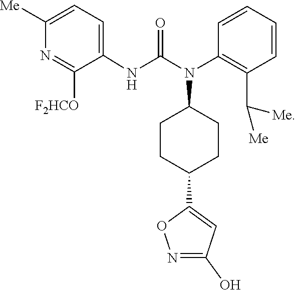 [SMILES: Cc1ccc(NC(=O)N(c2ccccc2C(C)C)[C@H]2CC[C@H](c3cc(O)no3)CC2)c(OC(F)F)n1]

In embodiments, the compound is

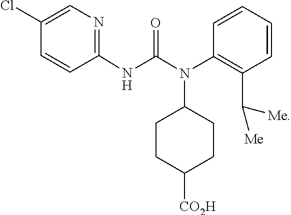 [SMILES: CC(C)c1ccccc1N(C(=O)Nc1ccc(Cl)cn1)C1CCC(C(=O)O)CC1]

In embodiments, the compound is

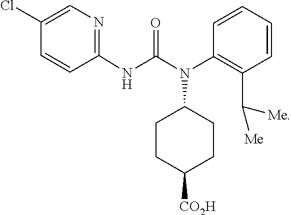 [SMILES: CC(C)c1ccccc1N(C(=O)Nc1ccc(Cl)cn1)[C@H]1CC[C@H](C(=O)O)CC1]

In embodiments, the compound is

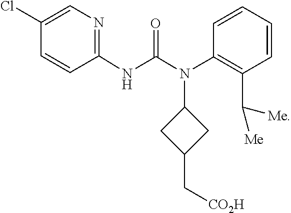 [SMILES: CC(C)c1ccccc1N(C(=O)Nc1ccc(Cl)cn1)C1CC(CC(=O)O)C1]

In embodiments, the compound

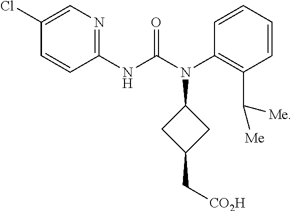 [SMILES: CC(C)c1ccccc1N(C(=O)Nc1ccc(Cl)cn1)[C@H]1C[C@@H](CC(=O)O)C1]

In embodiments, the compound is

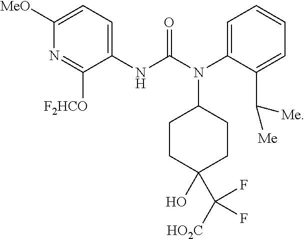 [SMILES: COc1ccc(NC(=O)N(c2ccccc2C(C)C)C2CCC(O)(C(F)(F)C(=O)O)CC2)c(OC(F)F)n1]

In embodiments, the compound is

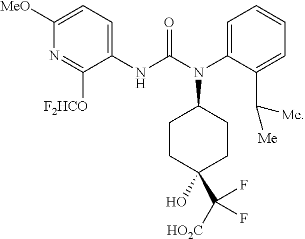 [SMILES: COc1ccc(NC(=O)N(c2ccccc2C(C)C)[C@H]2CC[C@@](O)(C(F)(F)C(=O)O)CC2)c(OC(F)F)n1]

In embodiments, the compound is

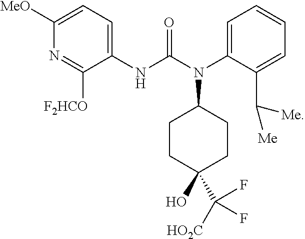 [SMILES: COc1ccc(NC(=O)N(c2ccccc2C(C)C)[C@H]2CC[C@](O)(C(F)(F)C(=O)O)CC2)c(OC(F)F)n1]

In embodiments, the compound is

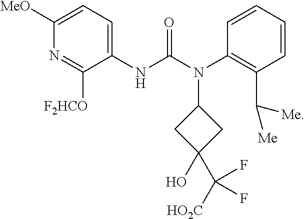 [SMILES: COc1ccc(NC(=O)N(c2ccccc2C(C)C)C2CC(O)(C(F)(F)C(=O)O)C2)c(OC(F)F)n1]

In embodiments, the compound is

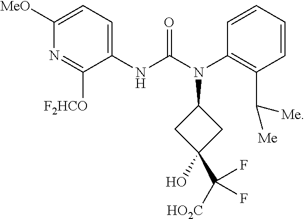 [SMILES: COc1ccc(NC(=O)N(c2ccccc2C(C)C)[C@H]2C[C@@](O)(C(F)(F)C(=O)O)C2)c(OC(F)F)n1]

In embodiments, the compound is

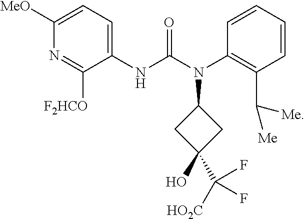 [SMILES: COc1ccc(NC(=O)N(c2ccccc2C(C)C)[C@H]2C[C@](O)(C(F)(F)C(=O)O)C2)c(OC(F)F)n1]

In embodiments, the compound is

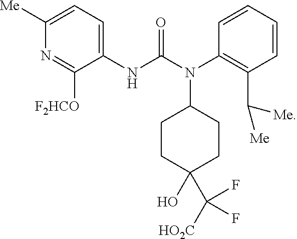 [SMILES: Cc1ccc(NC(=O)N(c2ccccc2C(C)C)C2CCC(O)(C(F)(F)C(=O)O)CC2)c(OC(F)F)n1]

In embodiments, the compound is

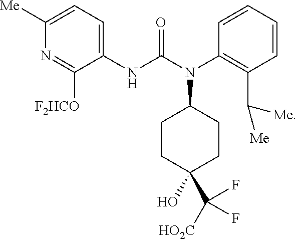 [SMILES: Cc1ccc(NC(=O)N(c2ccccc2C(C)C)[C@H]2CC[C@@](O)(C(F)(F)C(=O)O)CC2)c(OC(F)F)n1]

In embodiments, the compound is

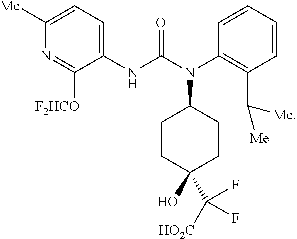 [SMILES: Cc1ccc(NC(=O)N(c2ccccc2C(C)C)[C@H]2CC[C@](O)(C(F)(F)C(=O)O)CC2)c(OC(F)F)n1]

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

In embodiments, R¹⁰ is not hydrogen, halogen, unsubstituted methyl, or unsubstituted alkoxy. In embodiments, R¹⁰ is not hydrogen. In embodiments, R¹⁰ is not halogen. In embodiments, R¹⁰ is not —F. In embodiments, R¹⁰ is not —Cl. In embodiments, R¹⁰ is not —CH₃. In embodiments, R¹⁰ is not substituted or unsubstituted heteroalkyl. In embodiments, R¹⁰ is not substituted or unsubstituted 2 to 6 membered heteroalkyl. In embodiments, R¹⁰ is not unsubstituted alkoxy. In embodiments, R¹⁰ is not unsubstituted methoxy. In embodiments, R¹⁰ is not unsubstituted ethoxy. In embodiments, R¹⁰ is not unsubstituted propoxy. In embodiments, R¹⁰ is not unsubstituted butoxy.

In embodiments, R¹⁰ and R² substituents are not joined to form a substituted or unsubstituted cycloalkyl, substituted or unsubstituted heterocycloalkyl, substituted or unsubstituted aryl, or substituted or unsubstituted heteroaryl. In embodiments, R¹⁰ and R² substituents are not joined to form a substituted or unsubstituted heteroaryl. In embodiments, R¹⁰ and R² substituents are not joined to form a substituted or unsubstituted 5 to 6 membered heteroaryl. In embodiments, R¹⁰ and R² substituents are not joined to form a methyl-substituted 5 to 6 membered heteroaryl. In embodiments, R¹⁰ and R² substituents are not joined to form

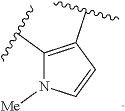 [SMILES: *c1ccn(C)c1*]

In embodiments, R³ is not halogen. In embodiments, R³ is not —F. In embodiments, R³ is not —Cl. In embodiments, R³ is not —OH. In embodiments, R³ is not —S(O)₂CH₃. In embodiments, R³ is not —CH₃.

In embodiments, R⁹ is not unsubstituted C₃-C₈ cycloalkyl. In embodiments, R⁹ is not unsubstituted cyclopropyl. In embodiments, R⁹ is not unsubstituted cyclobutyl. In embodiments, R⁹ is not unsubstituted cyclopentyl. In embodiments, R⁹ is not unsubstituted cyclobutyl. In embodiments, R⁹ is not unsubstituted cyclopentyl. In embodiments, R⁹ is not unsubstituted cyclohexyl. In embodiments, R⁹ is not unsubstituted cycloheptyl. In embodiments, R⁹ is not unsubstituted cyclooctyl.

In embodiments, -L¹-R⁹ is not

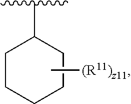

wherein R¹¹ and z11 are as described herein, including in embodiments. In embodiments, -L¹-R⁹ is not

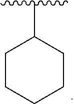 [SMILES: *C1CCCCC1]

In embodiments, -L¹-R⁹ is not

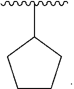 [SMILES: *C1CCCC1]

In embodiments, -L¹-R⁹ is not

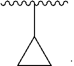 [SMILES: *C1CC1]

In embodiments, -L¹-R⁹ is not

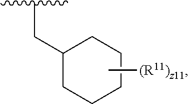

wherein R¹¹ and z11 are as described herein, including in embodiments. In embodiments, -L¹-R⁹ is not

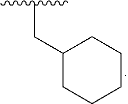 [SMILES: *CC1CCCCC1]

In embodiments, -L¹-R⁹ is not

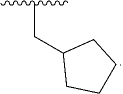 [SMILES: *CC1CCCC1]

In embodiments, -L¹-R⁹ is not

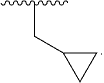 [SMILES: *CC1CC1]

In embodiments, -L¹-R⁹ is not

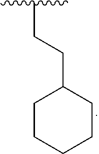 [SMILES: *CCC1CCCCC1]

In embodiments, -L¹-R⁹ is not

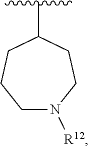

wherein R¹² is as described herein, including in embodiments. In embodiments, -L¹-R⁹ is not

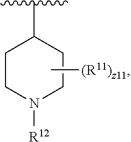

wherein R¹¹, z11, and R¹² are as described herein, including in embodiments.

In embodiments, -L¹-R⁹ is not

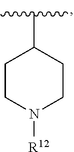

wherein R¹² is as described herein, including in embodiments. In embodiments, -L¹-R⁹ is not

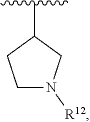

wherein R¹² is as described herein, including in embodiments. In embodiments, -L¹-R⁹ is not

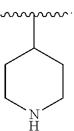 [SMILES: *C1CCNCC1]

In embodiments, -L¹-R⁹ is not

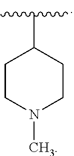 [SMILES: *C1CCN(C)CC1]

In embodiments, -L¹-R⁹ is not

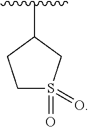 [SMILES: *C1CCS(=O)(=O)C1]

### III. Pharmaceutical Compositions

In an aspect is provided a pharmaceutical composition including a compound described herein, or a pharmaceutically acceptable salt or solvate thereof, and a pharmaceutically acceptable excipient.

In embodiments, the compound is a compound of formula (I), (I-1a), (I-1b), (I-1c), (I-1d), (I-2a), (I-2b), (I-2c), (I-2d), (I-3a), (I-3b), (I-3c), (I-3d), (I-4a), (I-4b), (I-4c), (I-4d), (II), (II-1a), (II-1b), (II-2a), (II-2b), (II-3a), or (II-3b). In embodiments, the compound is a compound of formula (I). In embodiments, the compound is a compound of formula (I-1a).

In embodiments, the compound is a compound of formula (I-1b). In embodiments, the compound is a compound of formula (I-1c). In embodiments, the compound is a compound of formula (I-1d). In embodiments, the compound is a compound of formula (I-2a). In embodiments, the compound is a compound of formula (I-2b). In embodiments, the compound is a compound of formula (I-2c). In embodiments, the compound is a compound of formula (I-2d). In embodiments, the compound is a compound of formula (I-3a). In embodiments, the compound is a compound of formula (I-3b). In embodiments, the compound is a compound of formula (I-3c). In embodiments, the compound is a compound of formula (I-3d). In embodiments, the compound is a compound of formula (I-4a). In embodiments, the compound is a compound of formula (I-4b). In embodiments, the compound is a compound of formula (I-4c). In embodiments, the compound is a compound of formula (I-4d). In embodiments, the compound is a compound of formula (II). In embodiments, the compound is a compound of formula (II-1a). In embodiments, the compound is a compound of formula (II-1b). In embodiments, the compound is a compound of formula (II-2a). In embodiments, the compound is a compound of formula (II-2b). In embodiments, the compound is a compound of formula (II-3a). In embodiments, the compound is a compound of formula (II-3b).

In embodiments, the pharmaceutical composition includes an effective amount of the compound. In embodiments, the pharmaceutical composition includes a therapeutically effective amount of the compound.

### IV. Methods of Use

In an aspect is provided a method of treating a neurodegenerative disorder in a subject in need thereof, the method including administering to the subject in need thereof a therapeutically effective amount of a compound described herein, or a pharmaceutically acceptable salt of solvate thereof.

In an aspect is provided a method of treating an inflammatory disease in a subject in need thereof, the method including administering to the subject in need thereof a therapeutically effective amount of a compound described herein, or a pharmaceutically acceptable salt of solvate thereof.

In embodiments, the inflammatory disease is encephalitis. In embodiments, the inflammatory disease is post-hemorrhagic encephalitis. In embodiments, the inflammatory disease is ocular inflammation. In embodiments, the inflammatory disease is conjunctivitis.

In embodiments, the inflammatory disease is allergic conjunctivitis. In embodiments, the inflammatory disease is vernal keratoconjunctivitis. In embodiments, the inflammatory disease is papillary conjunctivitis. In embodiments, the inflammatory disease is Sjogren's syndrome. In embodiments, the inflammatory disease is inflammatory disease with dry eyes.

In an aspect is provided a method of treating a demyelinating disease in a subject in need thereof, the method including administering to the subject in need thereof a therapeutically effective amount of a compound described herein, or a pharmaceutically acceptable salt of solvate thereof.

In embodiments, the demyelinating disease is a demyelinating disease of the central nervous system. In embodiments, the demyelinating disease is multiple sclerosis. In embodiments, the demyelinating disease is a demyelinating disease of the peripheral nervous system.

In an aspect is provided a method of treating fibrotic disease in a subject in need thereof, the method including administering to the subject in need thereof a therapeutically effective amount of a compound described herein, or a pharmaceutically acceptable salt of solvate thereof.

In embodiments, the fibrotic disease is pulmonary fibrosis. In embodiments, the fibrotic disease is skin fibrosis. In embodiments, the fibrotic disease is liver fibrosis. In embodiments, the fibrotic disease is ocular fibrosis. In embodiments, the fibrotic disease is idiopathic pulmonary fibrosis. In embodiments, the fibrotic disease is scleroderma. In embodiments, the fibrotic disease is nonalcoholic steatohepatitis. In embodiments, the fibrotic disease is ocular fibrosis. In embodiments, the fibrotic disease is hypertrophic scarring or keloids (e.g., burn induced or surgical, sarcoidosis, scleroderma, spinal cord injury/fibrosis, myelofibrosis, vascular restenosis, atherosclerosis, arteriosclerosis, Wegener's granulomatosis, mixed connective tissue disease, or Peyronie's disease). In embodiments, the fibrotic disease is iatrogenic pulmonary fibrosis. In embodiments, the fibrotic disease is radiation-induced fibrosis. In embodiments, the fibrotic disease is silicosis-induced pulmonary fibrosis. In embodiments, the fibrotic disease is asbestos-induced pulmonary fibrosis. In embodiments, the fibrotic disease is pleural fibrosis. In embodiments, the fibrotic disease is pulmonary fibrosis associated with SARS-CoV-2 infection and/or COVID-19. In embodiments, the fibrotic disease is pulmonary fibrosis secondary to systemic inflammatory disease. In embodiments, the fibrotic disease is pulmonary fibrosis secondary to sarcoidosis.

In embodiments, the fibrotic disease is gut fibrosis. In embodiments, the fibrotic disease is head and neck fibrosis. In embodiments, the fibrotic disease is cirrhosis. In embodiments, the fibrotic disease is alcohol-induced liver fibrosis. In embodiments, the fibrotic disease is endometriosis. In embodiments, the fibrotic disease is spinal cord fibrosis. In embodiments, the fibrotic disease is myelofibrosis. In embodiments, the fibrotic disease is cardiac fibrosis.
In embodiments, the fibrotic disease is perivascular fibrosis. In embodiments, the fibrotic disease is Peyronie's disease. In embodiments, the fibrotic disease is abdominal or bowel adhesions. In embodiments, the fibrotic disease is bladder fibrosis. In embodiments, the fibrotic disease is fibrosis of the nasal passages. In embodiments, the fibrotic disease is fibrosis mediated by fibroblasts. In embodiments, the fibrotic disease is renal fibrosis associated with chronic kidney disease (CKD).

In an aspect is provided a method of treating cancer in a subject in need thereof, the method including administering to the subject in need thereof a therapeutically effective amount of a compound described herein, or a pharmaceutically acceptable salt of solvate thereof.

In embodiments, the cancer is brain cancer. In embodiments, the cancer is glioblastoma. In embodiments, the cancer is a solid tumor (e.g., of the bladder, bowel, brain, breast, endometrium, heart, kidney, lung, lymphatic tissue (e.g., lymphoma), ovary, pancreas or other endocrine organ (e.g., thyroid), prostate, skin (e.g., melanoma or basal cell cancer)) or hematological tumors (e.g., leukemia) at any stage of the disease with or without metastases. In embodiments, the cancer is acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (e.g., osteosarcoma or malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-cell lymphoma, embryonal tumors, endometrial cancer, ependymoblastoma, ependymoma, esophageal cancer, Ewing sarcoma family of tumors, eye cancer, retinoblastoma, gallbladder cancer, gastrointestinal carcinoid tumor, gastrointestinal stromal tumor (GIST), germ cell tumor, glioma, hairy cell leukemia, head and neck cancer, hepatocellular cancer, Hodgkin lymphoma, hypopharyngeal cancer, intraocular melanoma, islet cell tumors (e.g., endocrine pancreas), Kaposi's sarcoma, kidney cancer, Langerhans cell histiocytosis, laryngeal cancer, leukemia, liver cancer, lymphoma, medulloblastoma, medulloepithelioma, melanoma, mesothelioma, mouth cancer, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, stomach (e.g., gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, or Wilms' tumor.

In an aspect is provided a method of treating an LPAR1-associated disease in a subject in need thereof, the method including administering to the subject in need thereof a therapeutically effective amount of a compound described herein, or a pharmaceutically acceptable salt of solvate thereof.

In embodiments, the LPAR1-associated disease is a neurodegenerative disease. In embodiments, the LPAR1-associated disease is an inflammatory disease. In embodiments, the LPAR1-associated disease is post-hemorrhagic encephalitis. In embodiments, the LPAR1-associated disease is a demyelinating disease. In embodiments, the LPAR1-associated disease is multiple sclerosis. In embodiments, the LPAR1-associated disease is a fibrotic disease. In embodiments, the LPAR1-associated disease is pulmonary fibrosis. In embodiments, the LPAR1-associated disease is idiopathic pulmonary fibrosis. In embodiments, the LPAR1-associated disease is cancer (e.g., brain cancer, ovarian cancer, colon cancer, prostate cancer, breast cancer, melanoma, head and neck cancer, bowel cancer, colorectal cancer, or thyroid cancer). In embodiments, the LPAR1-associated disease is pain (e.g., neuropathic pain, acute pain, or chronic pain).

In embodiments, the LPAR1-associated disease is a respiratory or allergic disorder.

In embodiments, the respiratory or allergic disorder is asthma, peribronchiolar fibrosis, obliterative bronchiolitis, or chronic obstructive pulmonary disease (COPD). In embodiments, the COPD is chronic bronchitis or emphysema, pulmonary hypertension, interstitial lung fibrosis and/or airway inflammation, or cystic fibrosis. In embodiments, the respiratory disease is adult respiratory distress syndrome or allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, and hypoxia.

In embodiments, the LPAR1-associated disease is a nervous system disorder. In embodiments, the nervous system disorder is Alzheimer's Disease, cerebral edema, cerebral ischemia, stroke, multiple sclerosis, neuropathies, Parkinson's Disease, a nervous condition found after blunt or surgical trauma (including post-surgical cognitive dysfunction and spinal cord or brain stem injury), degenerative disk disease, or sciatica.

In embodiments, the LPAR1-associated disease is a cardiovascular disorder. In embodiments, the cardiovascular disorder is arrhythmia (e.g., atrial or ventricular); atherosclerosis and its sequelae; angina; cardiac rhythm disturbances; myocardial ischemia; myocardial infarction; cardiac or vascular aneurysm; vasculitis; stroke; peripheral obstructive arteriopathy of a limb, an organ, or a tissue; reperfusion injury following ischemia of the brain, heart or other organ or tissue; endotoxic, surgical, or traumatic shock; hypertension; valvular heart disease; heart failure; abnormal blood pressure; shock; vasoconstriction (including that associated with migraines); vascular abnormality, or a cardiovascular insufficiency limited to a single organ or tissue.

In embodiments, the LPAR1-associated disease is lung fibrosis, kidney fibrosis, liver fibrosis, scarring, asthma, rhinitis, chronic obstructive pulmonary disease (COPD), pulmonary hypertension, interstitial lung fibrosis, arthritis, allergy, psoriasis, inflammatory bowel disease, adult respiratory distress syndrome, myocardial infarction, aneurysm, stroke, cancer, pain, proliferative disorders, or inflammatory conditions.

In embodiments, the LPAR1-associated disease is a liver disease. In embodiments, the liver disease is hepatitis C, liver cancer, familial combined hyperlipidemia, non-alcoholic fatty liver disease (NAFLD), progressive familial intrahepatic cholestasis, primary biliary cirrhosis (PBC), or primary sclerosing cholangitis (PSC). In embodiments, the liver disease is primary sclerosing cholangitis (PSC). In embodiments, the liver disease includes portal hypertension. In embodiments, liver cancer includes hepatocellular carcinoma (HCC), cholangiocarcinoma, angiosarcoma, or hemangiosarcoma. In embodiments, NAFLD includes steatosis. In embodiments, NAFLD includes NASH. In embodiments, NAFLD or NASH includes liver fibrosis. In embodiments, NAFLD or NASH includes liver cirrhosis.

In embodiments, NAFLD or NASH includes compensated liver cirrhosis. In embodiments, NAFLD or NASH includes decompensated liver fibrosis. In embodiments, NAFLD includes hepatocellular carcinoma (HCC). In embodiments, the liver disease is NASH.

In an aspect is provided a method of modulating LPAR1 activity in a subject, the method including administering to the subject a compound described herein, or a pharmaceutically acceptable salt or solvate thereof.

### V. Embodiments

Embodiment P1. A compound, or a pharmaceutically acceptable salt or solvate thereof, having the formula:

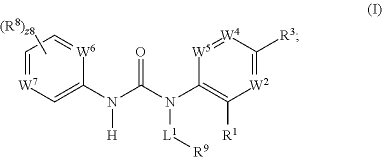

wherein
L¹ is a bond or substituted or unsubstituted C₁-C₅ alkylene;
R¹ is unsubstituted C₂-C₅ alkyl;
W² is N or C(R²);
R² is hydrogen, halogen, —CX²³, —CHX²², —CH₂X², —OCX²³, —OCH₂X², —OCHX²², —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —N^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶);
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —N^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —N^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R⁹ is substituted or unsubstituted cycloalkyl or substituted or unsubstituted heterocycloalkyl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, and R^{8D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, and X⁸ are independently —F, —Cl, —Br, or —I;
n2, n3, n4, n5, n6, n7, and n8 are independently an integer from 0 to 4;
m2, m3, m4, m5, m6, m7, m8, v2, v3, v4, v5, v6, v7, and v8 are independently 1 or 2; and
z8 is an integer from 0 to 3.

Embodiment P2. The compound of embodiment P1, having the formula:

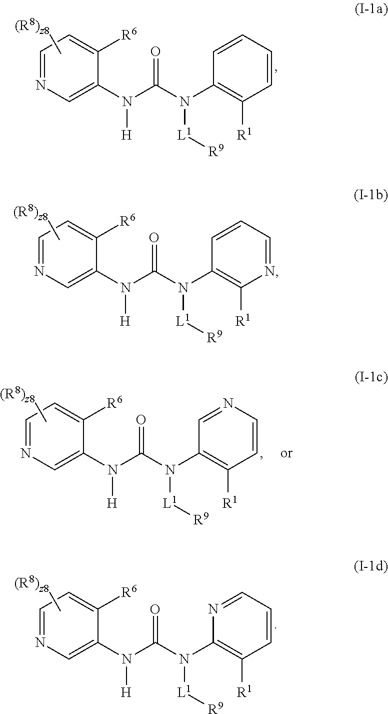

Embodiment P3. The compound of embodiment P1, having the formula:

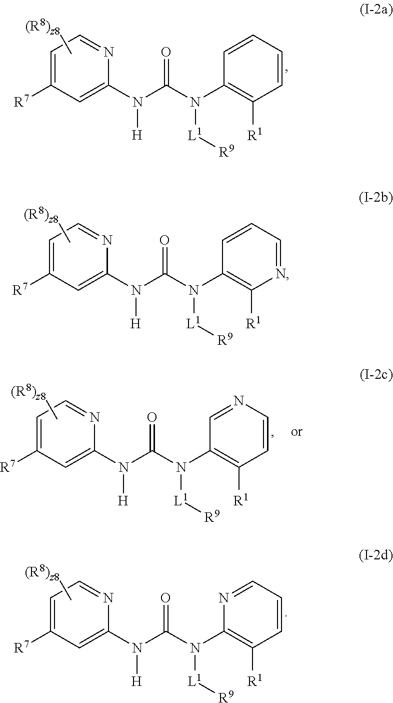

Embodiment P4. The compound of embodiment P1, having the formula:

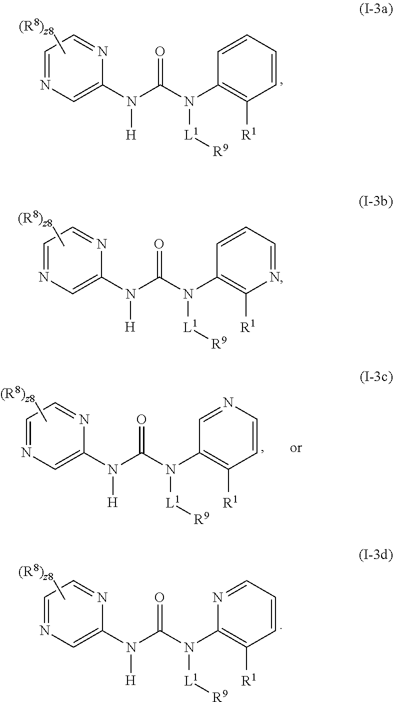

Embodiment P5. The compound of embodiment P1, having the formula:

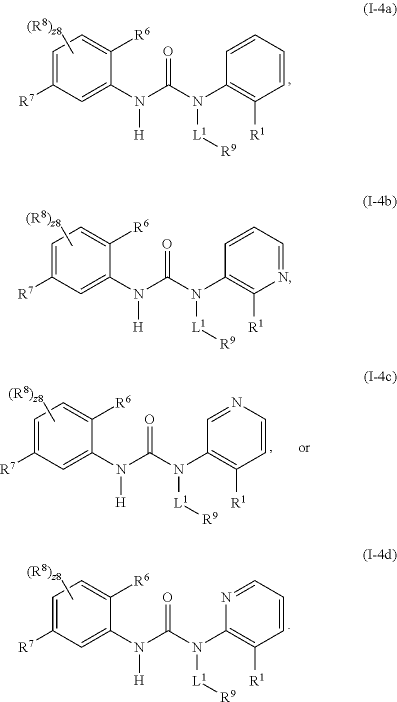

Embodiment P6. The compound of one of embodiments P1 to P5, wherein R¹ is unsubstituted C₃ alkyl.

Embodiment P7. The compound of one of embodiments P1 to P5, wherein R¹ is isopropyl.

Embodiment P8. A compound, or a pharmaceutically acceptable salt or solvate thereof, having the formula:

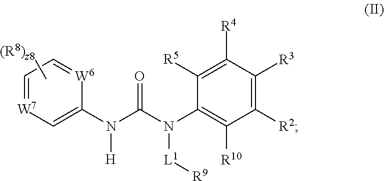

wherein
L¹ is a bond or substituted or unsubstituted C₁-C₅ alkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶);
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —N^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R⁹ is substituted or unsubstituted cycloalkyl or substituted or unsubstituted heterocycloalkyl;
R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)^{m10}, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A} OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, and X¹⁰ are independently —F, —Cl, —Br, or —I;
n2, n3, n4, n5, n6, n7, n8, and n10 are independently an integer from 0 to 4;
m2, m3, m4, m5, m6, m7, m8, m10, v2, v3, v4, v5, v6, v7, v8, and v10 are independently 1 or 2; and
z8 is an integer from 0 to 3;
wherein at least one of W⁶ or W⁷ is N;
wherein if W⁶ is C(R⁶) or W⁷ is C(R⁷), then R¹⁰ is not hydrogen;
wherein if W⁶ and W⁷ are both N, then R³ is not —S(O)₂CH₃; and
wherein if W⁶ is CH and W⁷ is N, then -L¹-R⁹ is not

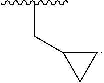 [SMILES: *CC1CC1]

Embodiment P9. The compound of embodiment P8, having the formula:

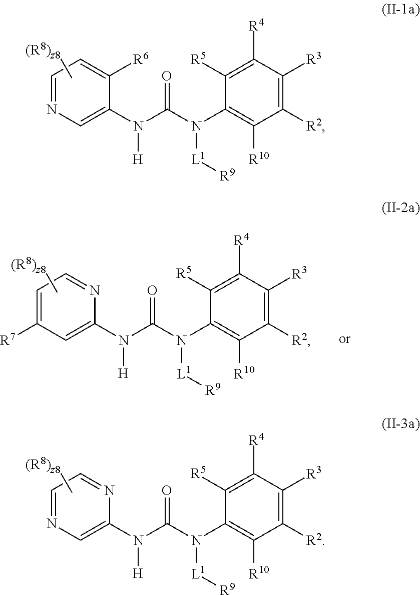

Embodiment P10. The compound of embodiment P8, having the formula:

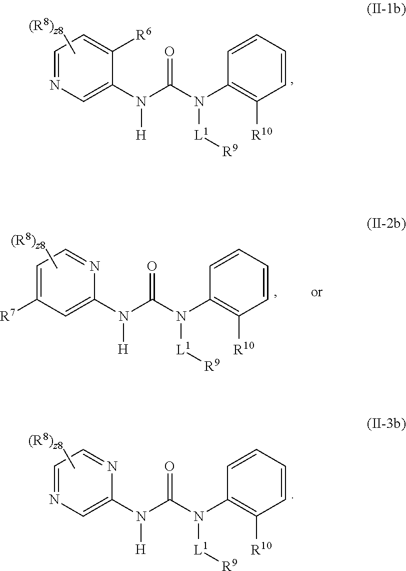

Embodiment P11. The compound of one of embodiments P8 to P10, wherein R¹⁰ is hydrogen or unsubstituted C₁-C₆ alkyl.

Embodiment P12. The compound of one of embodiments P8 to P10, wherein R¹⁰ is isopropyl.

Embodiment P13. The compound of one of embodiments P1 to P12, wherein R⁶ is hydrogen, —OCHF₂, unsubstituted C₁-C₆ alkyl, or unsubstituted 2 to 6 membered heteroalkyl.

Embodiment P14. The compound of one of embodiments P1 to P12, wherein R⁶ is hydrogen, —OCHF₂, unsubstituted methoxy, or unsubstituted isopropoxy.

Embodiment P15. The compound of one of embodiments P1 to P14, wherein R⁷ is hydrogen, —F, —Cl, —Br, or —OCHF₂.

Embodiment P16. The compound of one of embodiments P1 to P15, wherein R⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P17. The compound of one of embodiments P1 to P15, wherein R⁸ is independently halogen, —CF₃, —CHF₂, —CN, —OCHF₂, —C(O)R^{8C}, —C(O)OR^{8C}, —OR^{8D}, unsubstituted C₁-C₆ alkyl, unsubstituted 2 to 8 membered heteroalkyl, unsubstituted C₃-C₈ cycloalkyl, or unsubstituted phenyl.

Embodiment P18. The compound of embodiment P17, wherein R^{8C} is independently hydrogen or unsubstituted C₁-C₆ alkyl.

Embodiment P19. The compound of embodiment P17, wherein R^{8D} is independently unsubstituted C₁-C₆ alkyl.

Embodiment P20. The compound of one of embodiments P1 to P15, wherein R⁸ is independently —F, —Cl, —Br, —CF₃, —CHF₂, —CN, —C(O)H, —C(O)OCH₃, —OCHF₂, —OCH₃, —OCH₂CH₃, —OCH₂CF₃, —OCH(CH₃)CH₂OCH₃, —OCH₂CHF₂, unsubstituted methyl, unsubstituted cyclopropyl, or unsubstituted phenyl.

Embodiment P21. The compound of one of embodiments P1 to P15, wherein two R⁸ substituents are joined to form an unsubstituted C₅ cycloalkyl.

Embodiment P22. The compound of one of embodiments P1 to P21, wherein L¹ is a bond or unsubstituted C₁-C₅ alkylene.

Embodiment P23. The compound of one of embodiments P1 to P21, wherein L¹ is a bond.

Embodiment P24. The compound of one of embodiments P1 to P21, wherein L¹ is unsubstituted methylene.

Embodiment P25. The compound of one of embodiments P1 to P24, wherein R⁹ is an R¹¹-substituted or unsubstituted cycloalkyl or R¹¹-substituted or unsubstituted heterocycloalkyl;
R¹¹ is independently oxo, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR^{11C}N^{11A}R^{11B}, —ONR^{11A}R^{11B}, —NHC(O)NR^{11C}NR^{11A}R^{11B}, —NHC(O)NR^{11A}R^{11B}, —N(O)ₘ₁₁, —NR^{11A}R^{11B}, —C(O)R^{11C}, —C(O)OR^{11C}, —C(O)NR^{11A}R^{11B}, —OR^{11D}, —SR^{11D}, —NR^{11A}SO₂R^{11D}, —NR^{11A}C(O)R^{11C}, —NR^{11A}C(O)OR^{11C}, —NR^{11A}OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{11A}, R^{11B}, R^{11C}, and R^{11D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl;
X¹¹ is independently —F, —Cl, —Br, or —I;
n11 is independently an integer from 0 to 4; and
m11 and v11 are independently 1 or 2.

Embodiment P26. The compound of embodiment P25, wherein R⁹ is an R¹¹-substituted or unsubstituted C₃-C₈ cycloalkyl or R¹¹-substituted or unsubstituted 3 to 8 membered heterocycloalkyl.

Embodiment P27. The compound of embodiment P25, wherein R⁹ is an R¹¹-substituted or unsubstituted spirocyclic cycloalkyl or R¹¹-substituted or unsubstituted spirocyclic heterocycloalkyl.

Embodiment P28. The compound of embodiment P25, wherein R⁹ is

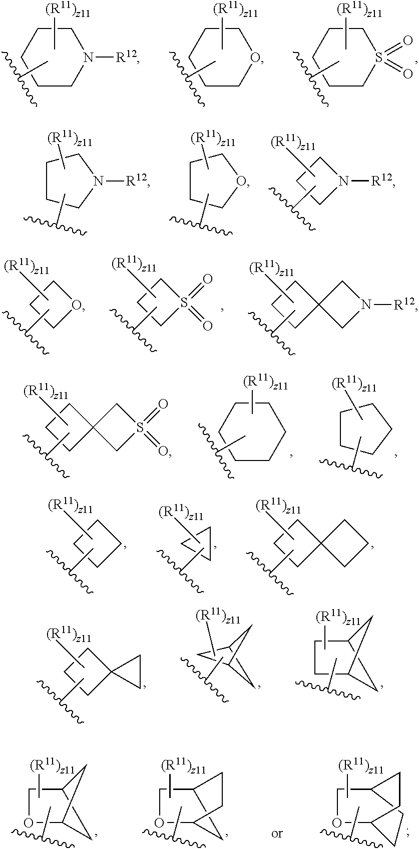

R¹² is hydrogen, halogen, —CX¹²₃, —CHX¹²₂, —CH₂X¹², —OCX¹²₃, —OCH₂X¹², —OCHX¹²₂, —SOₙ₁₂R^{12D}, —SOᵥ₁₂NR^{12A}R^{12B}, —C(O)R^{12C}, —C(O)OR^{12C}, —C(O)NR^{12A}R^{12B}, —OR^{12D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{12A}, R^{12B}, R^{12C}, and R^{12D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl;
X¹² is independently —F, —Cl, —Br, or —I;
n12 is independently an integer from 0 to 4;
v12 is independently 1 or 2; and
z11 is an integer from 0 to 13.

Embodiment P29. The compound of embodiment P28, wherein R⁹ is

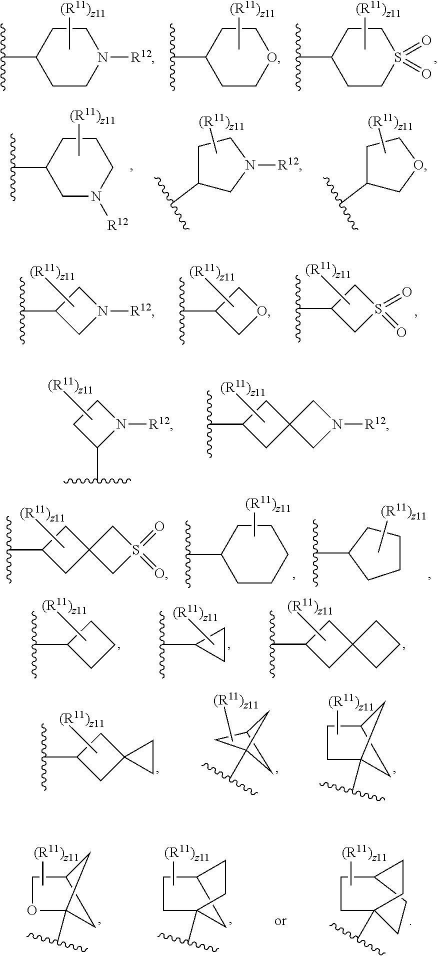

Embodiment P30. The compound of one of embodiments P25 to P29, wherein R¹¹ is independently oxo, halogen, —CX¹¹₃, —CN, —C(O)OR^{11C}, —C(O)NR^{11A}R^{11B}, —C(O)R^{11C}, —OR^{11D}, substituted or unsubstituted C₁-C₆ alkyl, or substituted or unsubstituted 2 to 5 membered heteroalkyl.

Embodiment P31. The compound of embodiment P30, wherein R^{11A} is independently hydrogen or unsubstituted C₁-C₆ alkyl; and R^{11B} is independently hydrogen.

Embodiment P32. The compound of embodiment P30, wherein R^{11C} is independently hydrogen or unsubstituted C₁-C₆ alkyl.

Embodiment P33. The compound of embodiment P30, wherein R^{11D} is independently hydrogen or unsubstituted C₁-C₆ alkyl.

Embodiment P34. The compound of one of embodiments P25 to P29, wherein two R¹¹ substituents are joined to form a substituted or unsubstituted cycloalkyl.

Embodiment P35. The compound of one of embodiments P28 to P29, wherein R¹² is hydrogen, —C(O)R^{12C}, —SOₙ₁₂R^{12D}, —SOᵥ₁₂NR^{12A}R^{12B}, —C(O)OR^{12C}, —C(O)NR^{12A}R^{12B}, unsubstituted C₁-C₆ alkyl, unsubstituted C₃-C₈ cycloalkyl, or unsubstituted 3 to 8 membered heterocycloalkyl.

Embodiment P36. The compound of embodiment P35, wherein R^{12A} is hydrogen, unsubstituted C₁-C₆ alkyl, or unsubstituted C₃-C₈ cycloalkyl; and R^{12B} is hydrogen.

Embodiment P37. The compound of embodiment P35, wherein R^{12C} is substituted or unsubstituted C₁-C₆ alkyl, substituted or unsubstituted 2 to 6 membered heteroalkyl, or substituted or unsubstituted C₃-C₈ cycloalkyl.

Embodiment P38. The compound of embodiment P35, wherein R^{12D} is unsubstituted C₁-C₆ alkyl.

Embodiment P39. The compound of one of embodiments P1 to P24, wherein R⁹ is

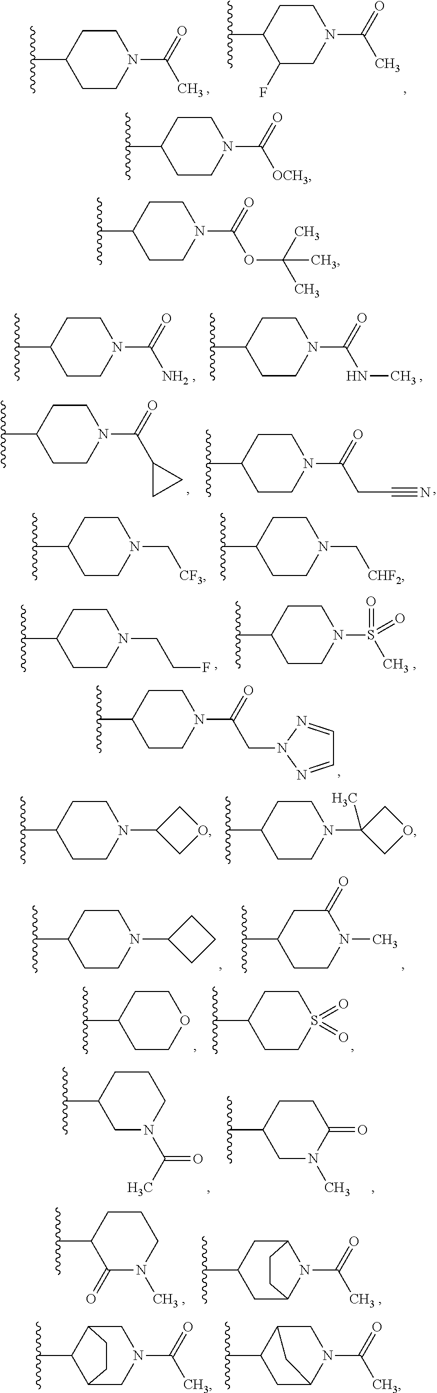

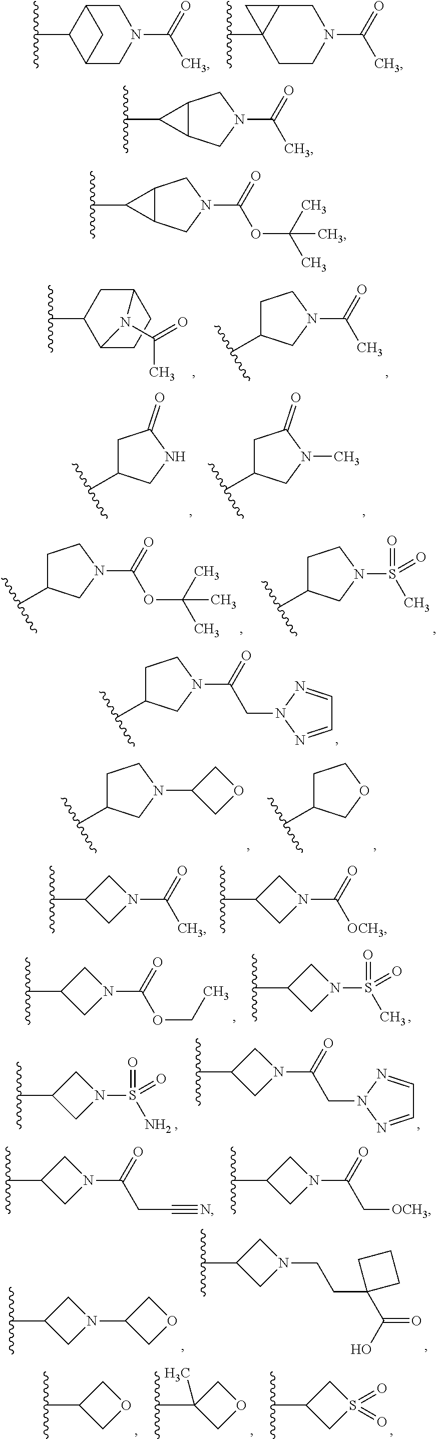

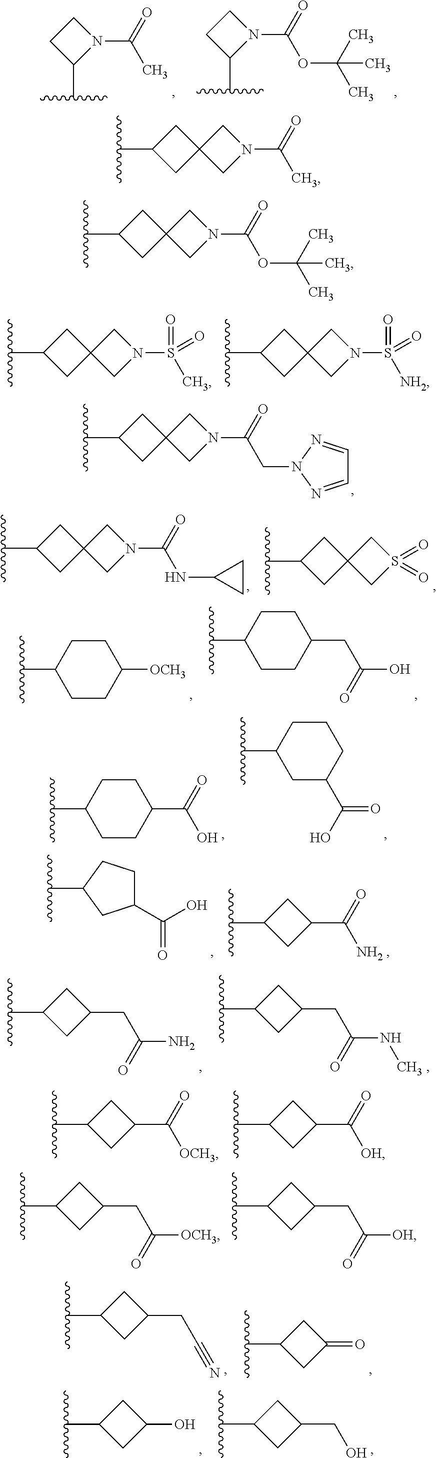

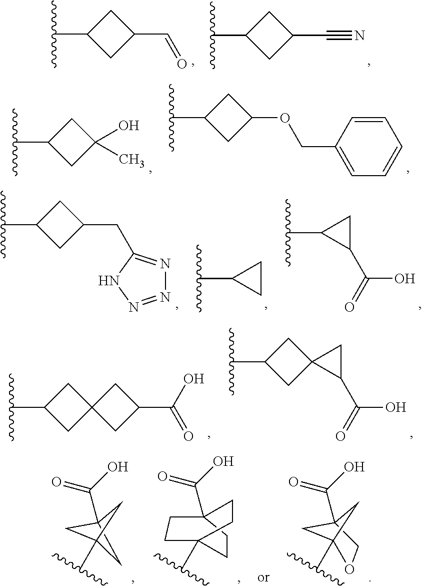

Embodiment P40. A compound, or a pharmaceutically acceptable salt or solvate thereof, having the formula:

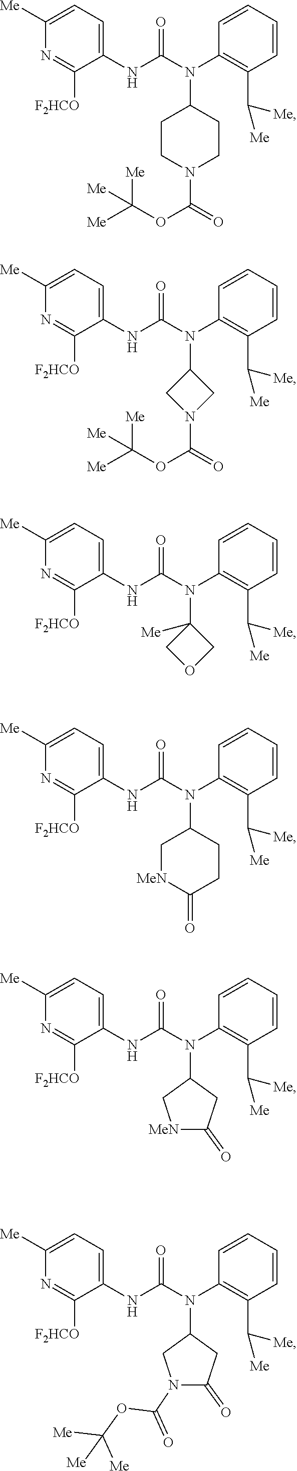 [SMILES: Cc1ccc(NC(=O)N(c2ccccc2C(C)C)C2CCN(C(=O)OC(C)(C)C)CC2)c(OC(F)F)n1 ; Cc1ccc(NC(=O)N(c2ccccc2C(C)C)C2CN(C(=O)OC(C)(C)C)C2)c(OC(F)F)n1 ; Cc1ccc(NC(=O)N(c2ccccc2C(C)C)C2(C)COC2)c(OC(F)F)n1 ; Cc1ccc(NC(=O)N(c2ccccc2C(C)C)C2CCC(=O)N(C)C2)c(OC(F)F)n1 ; Cc1ccc(NC(=O)N(c2ccccc2C(C)C)C2CC(=O)N(C)C2)c(OC(F)F)n1 ; Cc1ccc(NC(=O)N(c2ccccc2C(C)C)C2CC(=O)N(C(=O)OC(C)(C)C)C2)c(OC(F)F)n1]

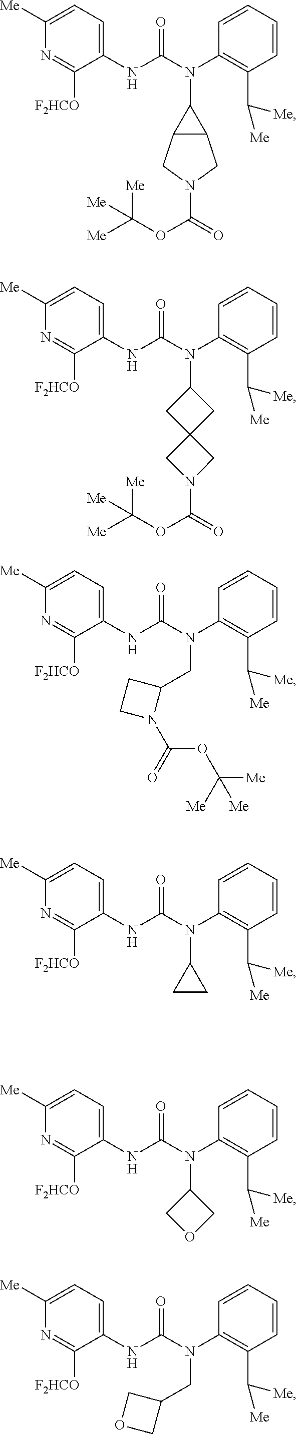 [SMILES: Cc1ccc(NC(=O)N(c2ccccc2C(C)C)C2C3CN(C(=O)OC(C)(C)C)CC32)c(OC(F)F)n1 ; Cc1ccc(NC(=O)N(c2ccccc2C(C)C)C2CC3(C2)CN(C(=O)OC(C)(C)C)C3)c(OC(F)F)n1 ; Cc1ccc(NC(=O)N(CC2CCN2C(=O)OC(C)(C)C)c2ccccc2C(C)C)c(OC(F)F)n1 ; Cc1ccc(NC(=O)N(c2ccccc2C(C)C)C2CC2)c(OC(F)F)n1 ; Cc1ccc(NC(=O)N(c2ccccc2C(C)C)C2COC2)c(OC(F)F)n1 ; Cc1ccc(NC(=O)N(CC2COC2)c2ccccc2C(C)C)c(OC(F)F)n1]

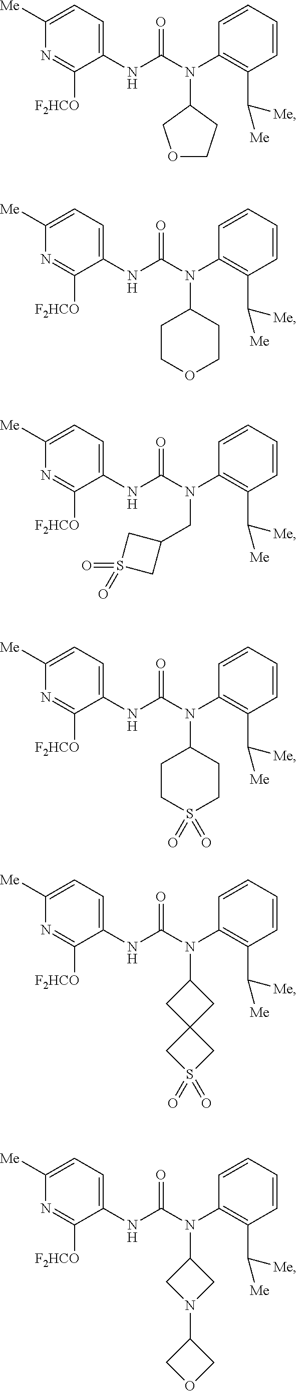 [SMILES: Cc1ccc(NC(=O)N(c2ccccc2C(C)C)C2CCOC2)c(OC(F)F)n1 ; Cc1ccc(NC(=O)N(c2ccccc2C(C)C)C2CCOCC2)c(OC(F)F)n1 ; Cc1ccc(NC(=O)N(CC2CS(=O)(=O)C2)c2ccccc2C(C)C)c(OC(F)F)n1 ; Cc1ccc(NC(=O)N(c2ccccc2C(C)C)C2CCS(=O)(=O)CC2)c(OC(F)F)n1 ; Cc1ccc(NC(=O)N(c2ccccc2C(C)C)C2CC3(C2)CS(=O)(=O)C3)c(OC(F)F)n1 ; Cc1ccc(NC(=O)N(c2ccccc2C(C)C)C2CN(C3COC3)C2)c(OC(F)F)n1]

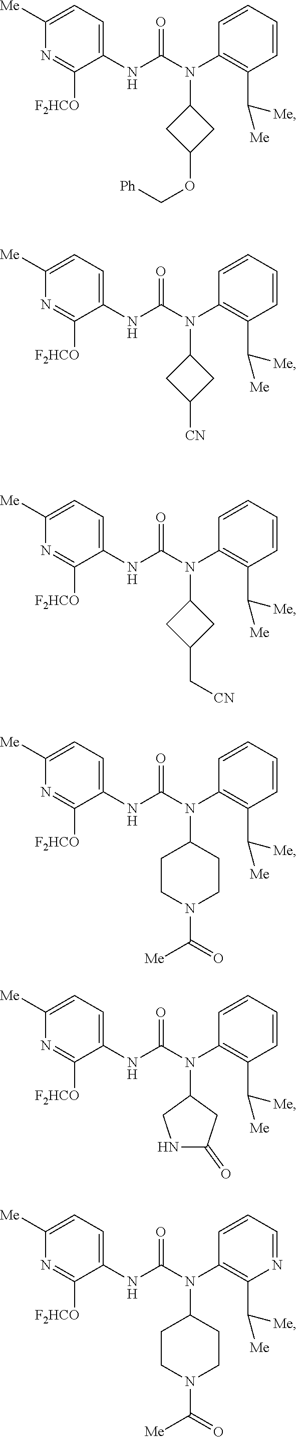

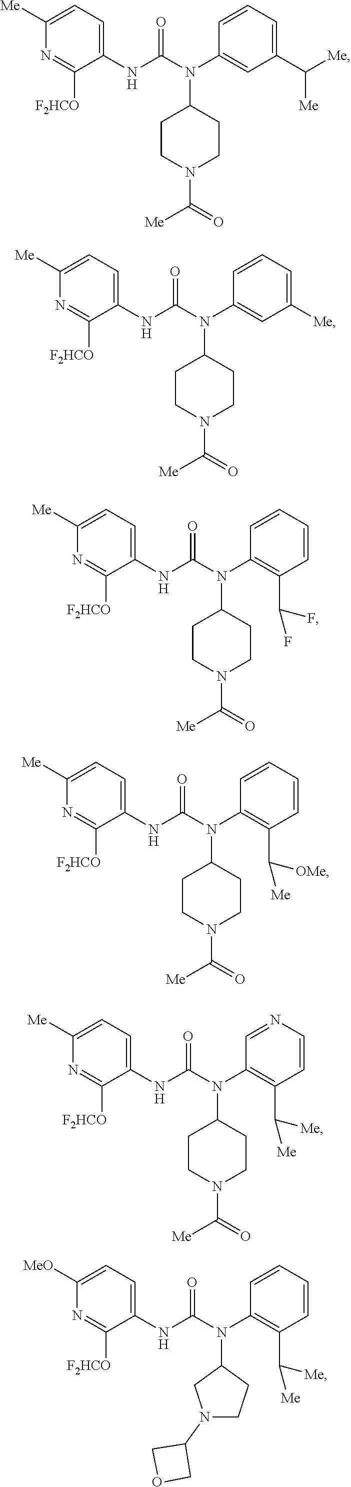

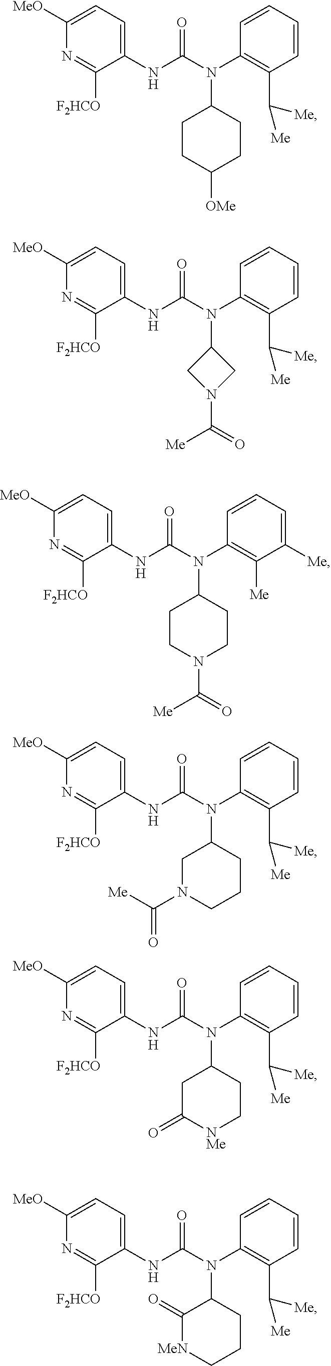 [SMILES: COc1ccc(NC(=O)N(c2ccccc2C(C)C)C2CCC(OC)CC2)c(OC(F)F)n1 ; COc1ccc(NC(=O)N(c2ccccc2C(C)C)C2CN(C(C)=O)C2)c(OC(F)F)n1 ; COc1ccc(NC(=O)N(c2cccc(C)c2C)C2CCN(C(C)=O)CC2)c(OC(F)F)n1 ; COc1ccc(NC(=O)N(c2ccccc2C(C)C)C2CCCN(C(C)=O)C2)c(OC(F)F)n1 ; COc1ccc(NC(=O)N(c2ccccc2C(C)C)C2CCN(C)C(=O)C2)c(OC(F)F)n1 ; COc1ccc(NC(=O)N(c2ccccc2C(C)C)C2CCCN(C)C2=O)c(OC(F)F)n1]

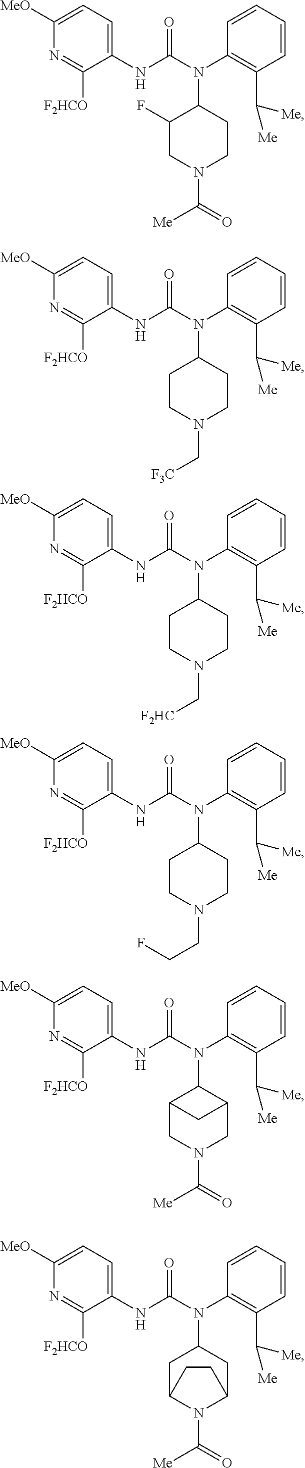

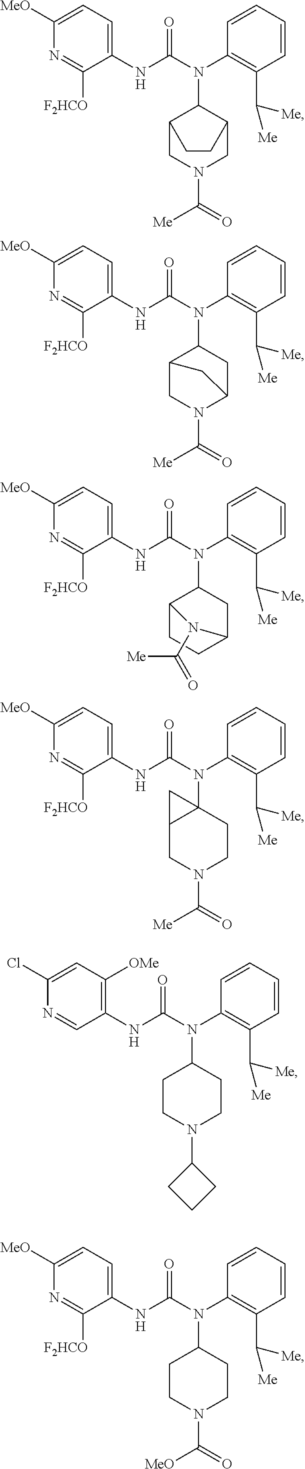

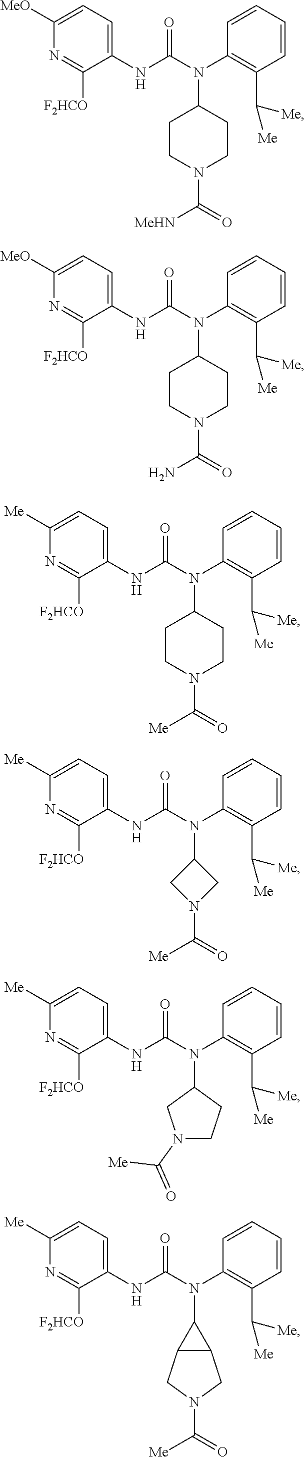

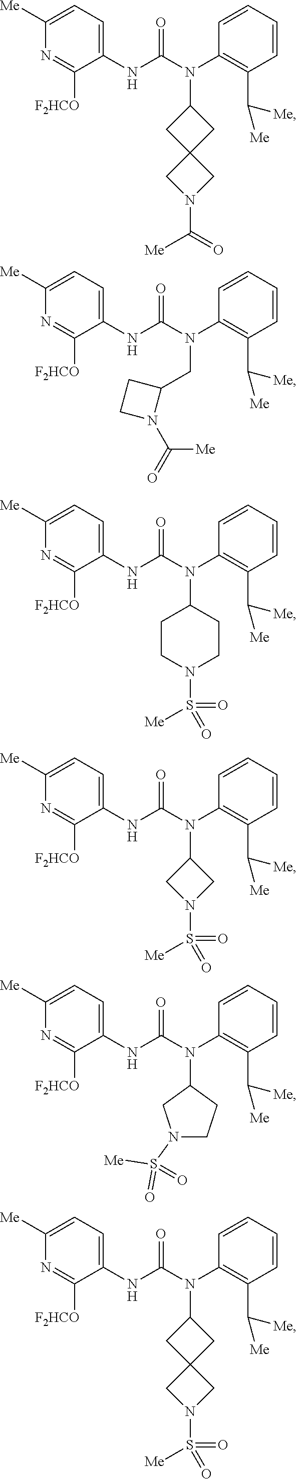

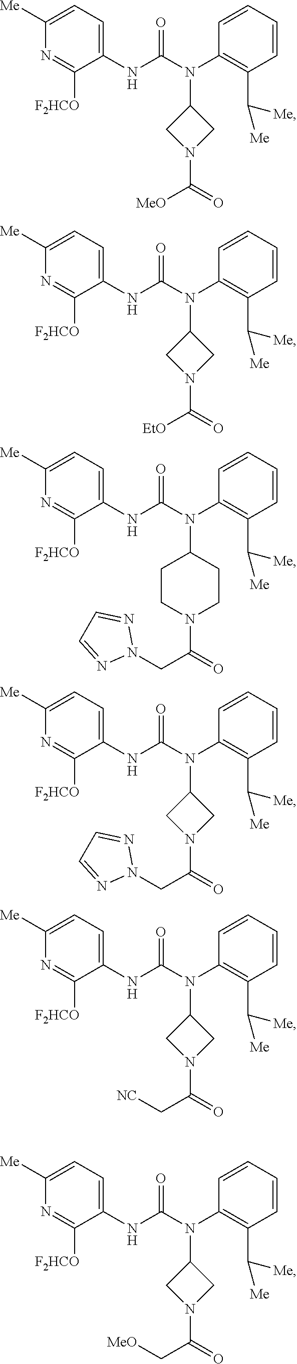

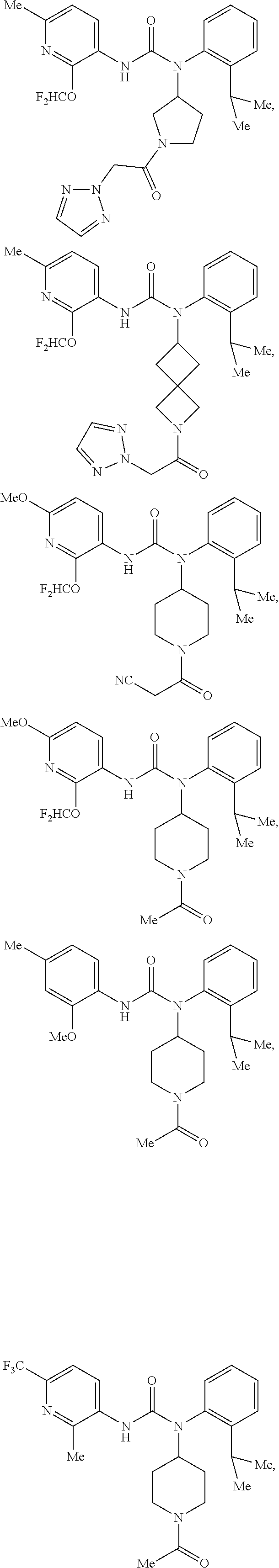

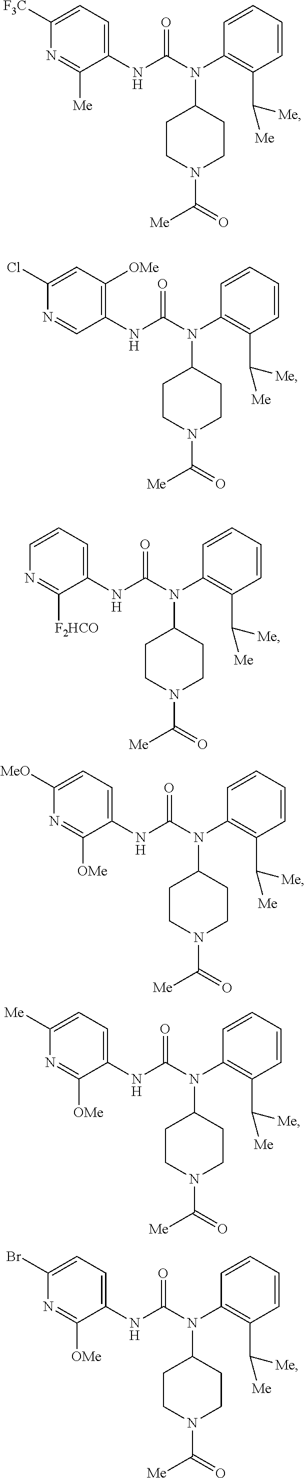

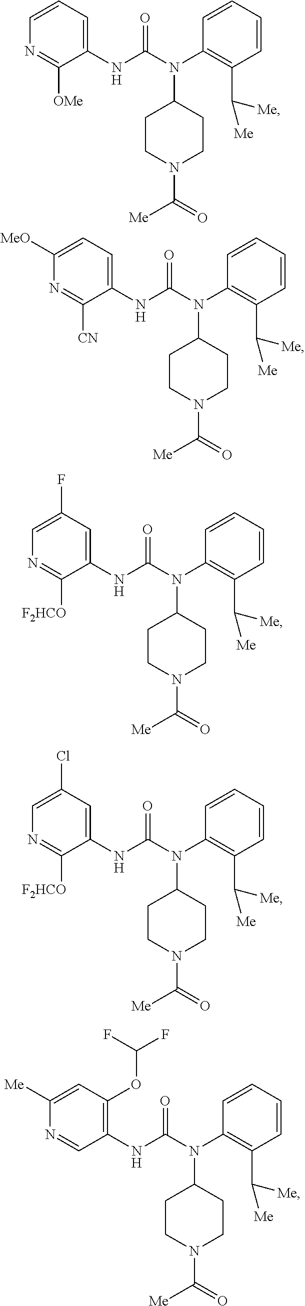

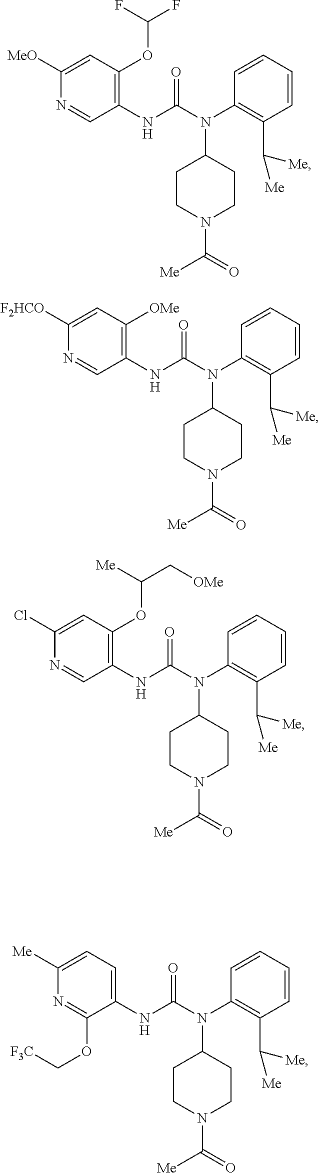

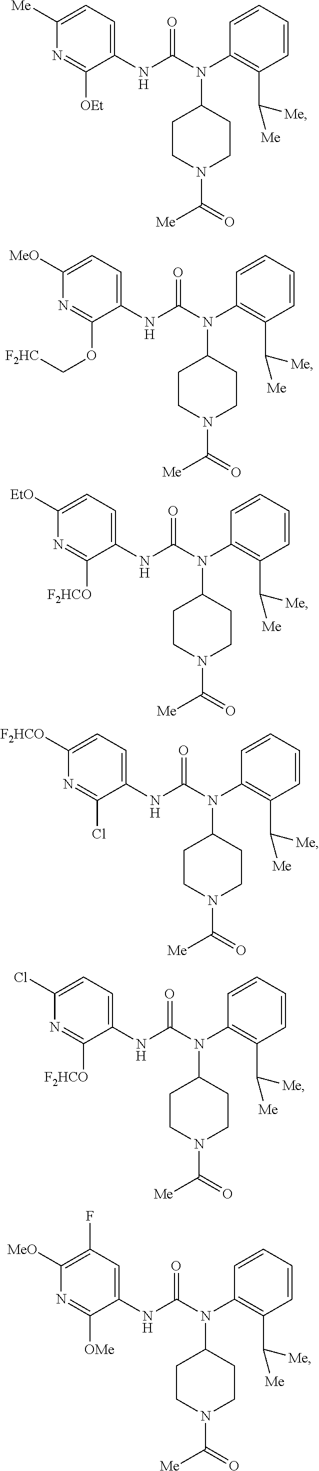

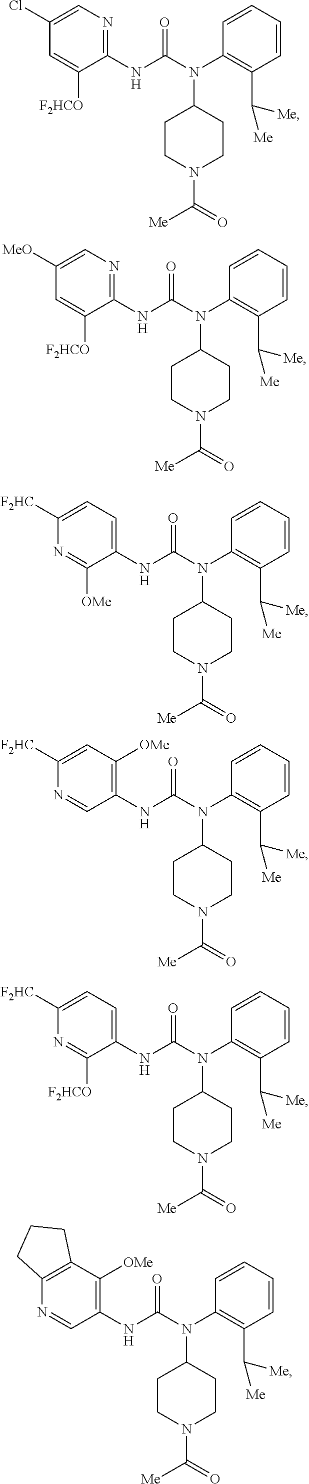

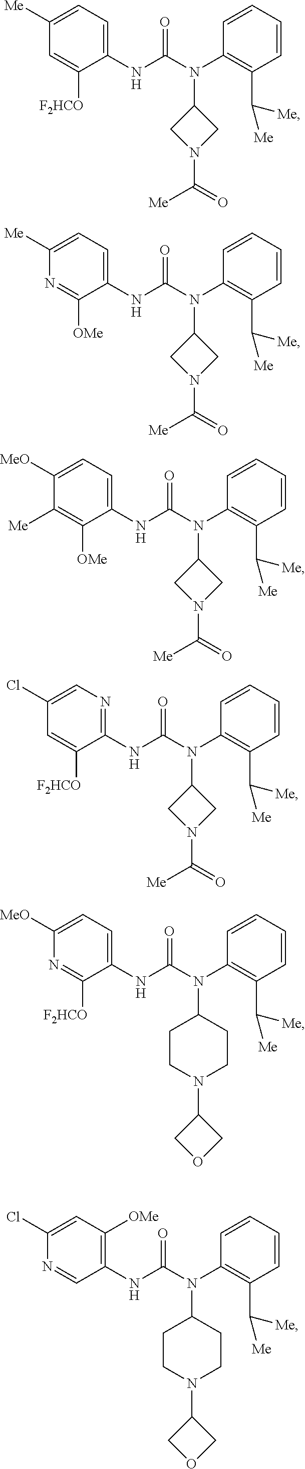

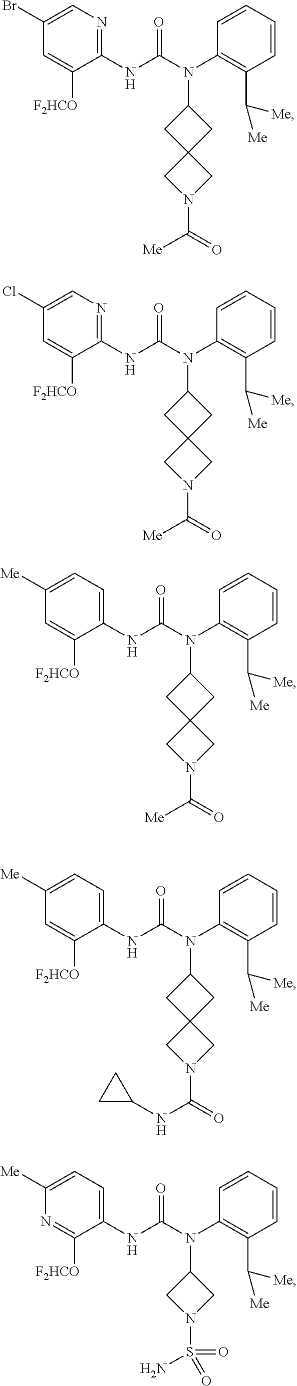 [SMILES: CC(=O)N1CC2(CC(N(C(=O)Nc3ncc(Br)cc3OC(F)F)c3ccccc3C(C)C)C2)C1 ; CC(=O)N1CC2(CC(N(C(=O)Nc3ncc(Cl)cc3OC(F)F)c3ccccc3C(C)C)C2)C1 ; CC(=O)N1CC2(CC(N(C(=O)Nc3ccc(C)cc3OC(F)F)c3ccccc3C(C)C)C2)C1 ; Cc1ccc(NC(=O)N(c2ccccc2C(C)C)C2CC3(C2)CN(C(=O)NC2CC2)C3)c(OC(F)F)c1 ; Cc1ccc(NC(=O)N(c2ccccc2C(C)C)C2CN(S(N)(=O)=O)C2)c(OC(F)F)n1]

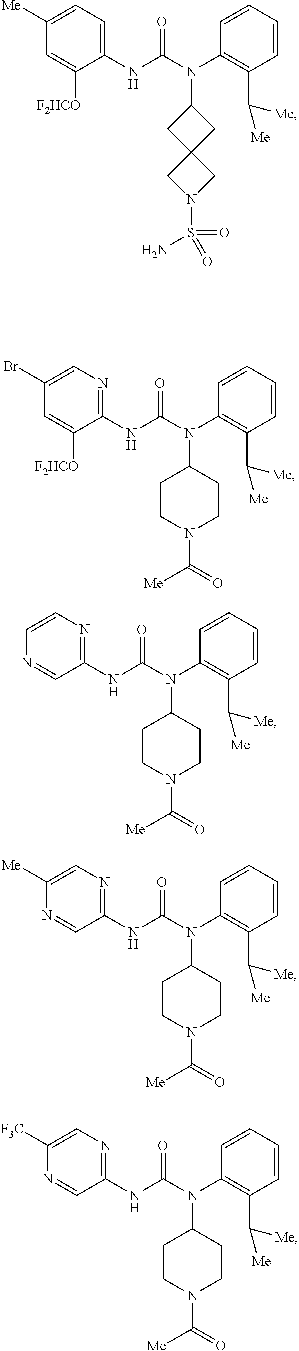

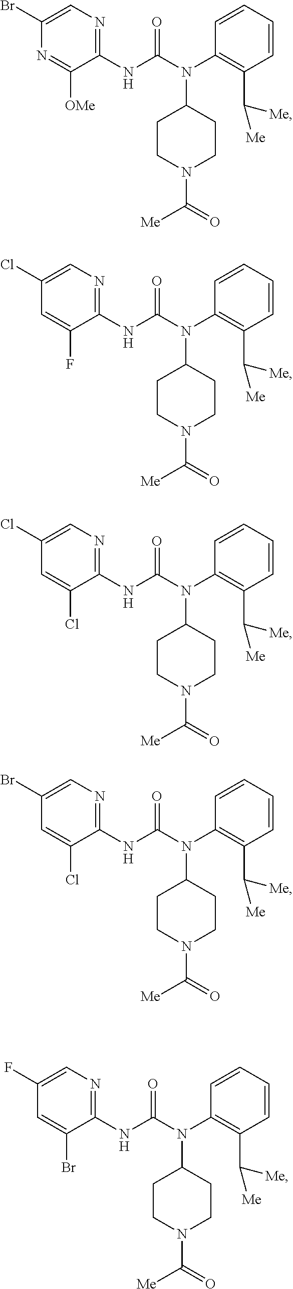 [SMILES: COc1nc(Br)cnc1NC(=O)N(c1ccccc1C(C)C)C1CCN(C(C)=O)CC1 ; CC(=O)N1CCC(N(C(=O)Nc2ncc(Cl)cc2F)c2ccccc2C(C)C)CC1 ; CC(=O)N1CCC(N(C(=O)Nc2ncc(Cl)cc2Cl)c2ccccc2C(C)C)CC1 ; CC(=O)N1CCC(N(C(=O)Nc2ncc(Br)cc2Cl)c2ccccc2C(C)C)CC1 ; CC(=O)N1CCC(N(C(=O)Nc2ncc(F)cc2Br)c2ccccc2C(C)C)CC1]

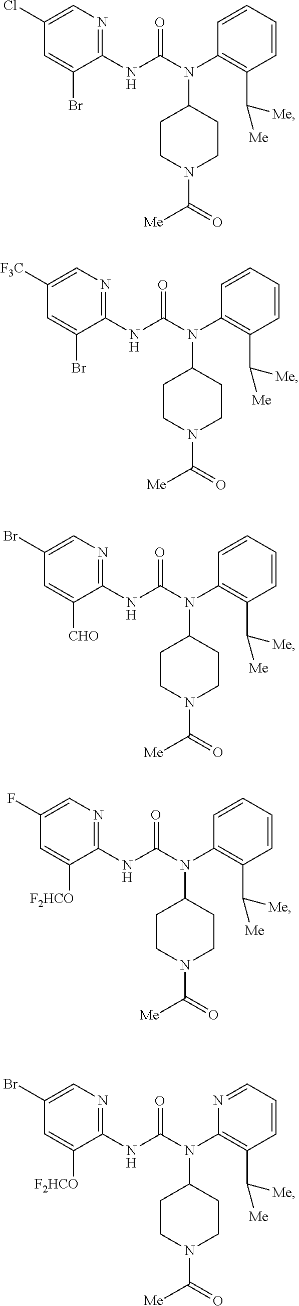 [SMILES: CC(=O)N1CCC(N(C(=O)Nc2ncc(Cl)cc2Br)c2ccccc2C(C)C)CC1 ; CC(=O)N1CCC(N(C(=O)Nc2ncc(C(F)(F)F)cc2Br)c2ccccc2C(C)C)CC1 ; CC(=O)N1CCC(N(C(=O)Nc2ncc(Br)cc2C=O)c2ccccc2C(C)C)CC1 ; CC(=O)N1CCC(N(C(=O)Nc2ncc(F)cc2OC(F)F)c2ccccc2C(C)C)CC1 ; CC(=O)N1CCC(N(C(=O)Nc2ncc(Br)cc2OC(F)F)c2ncccc2C(C)C)CC1]

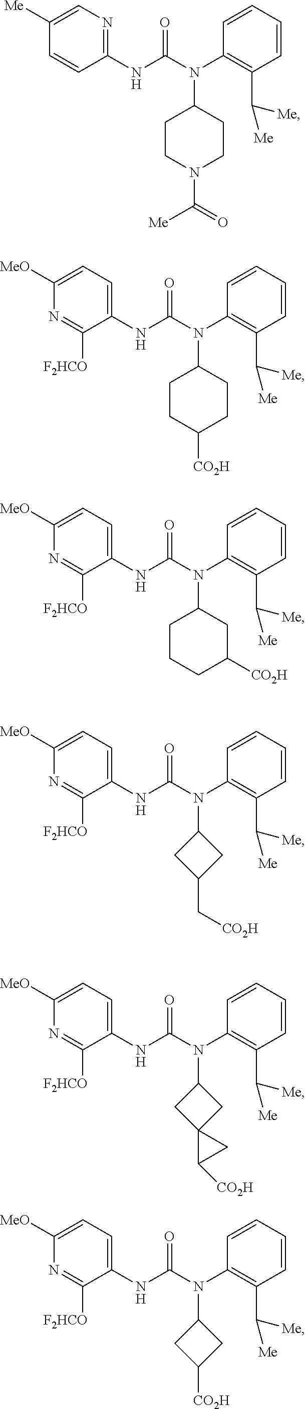 [SMILES: CC(=O)N1CCC(N(C(=O)Nc2ccc(C)cn2)c2ccccc2C(C)C)CC1 ; COc1ccc(NC(=O)N(c2ccccc2C(C)C)C2CCC(C(=O)O)CC2)c(OC(F)F)n1 ; COc1ccc(NC(=O)N(c2ccccc2C(C)C)C2CCCC(C(=O)O)C2)c(OC(F)F)n1 ; COc1ccc(NC(=O)N(c2ccccc2C(C)C)C2CC(CC(=O)O)C2)c(OC(F)F)n1 ; COc1ccc(NC(=O)N(c2ccccc2C(C)C)C2CC3(C2)CC3C(=O)O)c(OC(F)F)n1 ; COc1ccc(NC(=O)N(c2ccccc2C(C)C)C2CC(C(=O)O)C2)c(OC(F)F)n1]

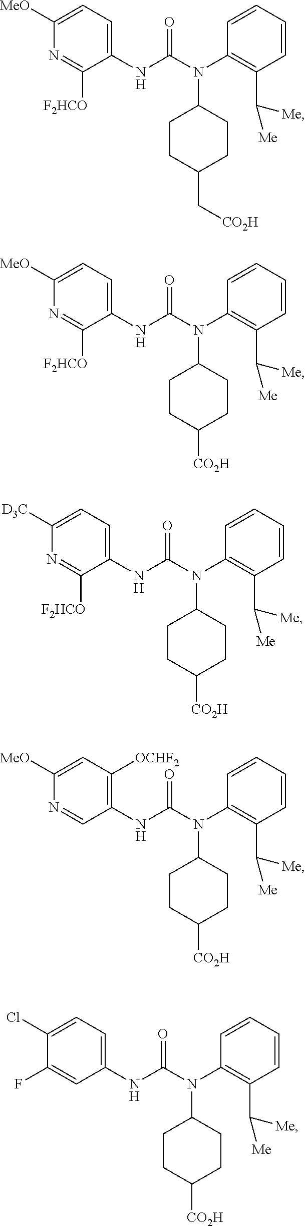 [SMILES: COc1ccc(NC(=O)N(c2ccccc2C(C)C)C2CCC(CC(=O)O)CC2)c(OC(F)F)n1 ; COc1ccc(NC(=O)N(c2ccccc2C(C)C)C2CCC(C(=O)O)CC2)c(OC(F)F)n1 ; [2H]C([2H])([2H])c1ccc(NC(=O)N(c2ccccc2C(C)C)C2CCC(C(=O)O)CC2)c(OC(F)F)n1 ; COc1cc(OC(F)F)c(NC(=O)N(c2ccccc2C(C)C)C2CCC(C(=O)O)CC2)cn1 ; CC(C)c1ccccc1N(C(=O)Nc1ccc(Cl)c(F)c1)C1CCC(C(=O)O)CC1]

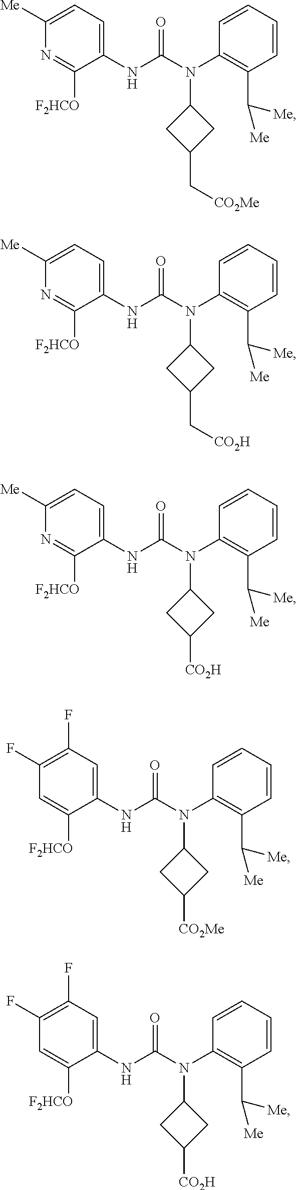 [SMILES: COC(=O)CC1CC(N(C(=O)Nc2ccc(C)nc2OC(F)F)c2ccccc2C(C)C)C1 ; Cc1ccc(NC(=O)N(c2ccccc2C(C)C)C2CC(CC(=O)O)C2)c(OC(F)F)n1 ; Cc1ccc(NC(=O)N(c2ccccc2C(C)C)C2CC(C(=O)O)C2)c(OC(F)F)n1 ; COC(=O)C1CC(N(C(=O)Nc2cc(F)c(F)cc2OC(F)F)c2ccccc2C(C)C)C1 ; CC(C)c1ccccc1N(C(=O)Nc1cc(F)c(F)cc1OC(F)F)C1CC(C(=O)O)C1]

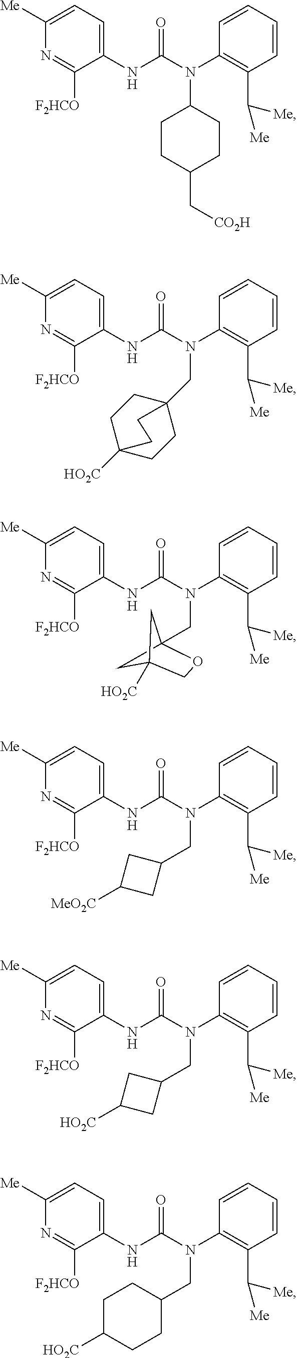 [SMILES: Cc1ccc(NC(=O)N(c2ccccc2C(C)C)C2CCC(CC(=O)O)CC2)c(OC(F)F)n1 ; Cc1ccc(NC(=O)N(CC23CCC(C(=O)O)(CC2)CC3)c2ccccc2C(C)C)c(OC(F)F)n1 ; Cc1ccc(NC(=O)N(CC23CC(C(=O)O)(CO2)C3)c2ccccc2C(C)C)c(OC(F)F)n1 ; COC(=O)C1CC(CN(C(=O)Nc2ccc(C)nc2OC(F)F)c2ccccc2C(C)C)C1 ; Cc1ccc(NC(=O)N(CC2CC(C(=O)O)C2)c2ccccc2C(C)C)c(OC(F)F)n1 ; Cc1ccc(NC(=O)N(CC2CCC(C(=O)O)CC2)c2ccccc2C(C)C)c(OC(F)F)n1]

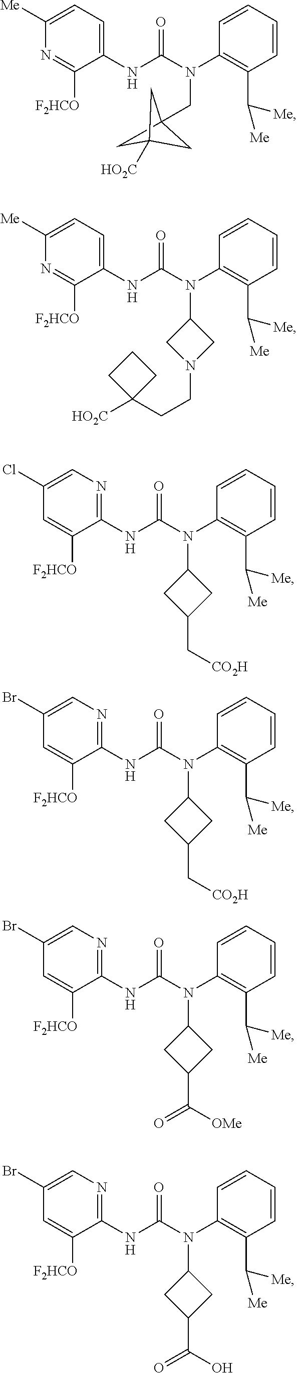

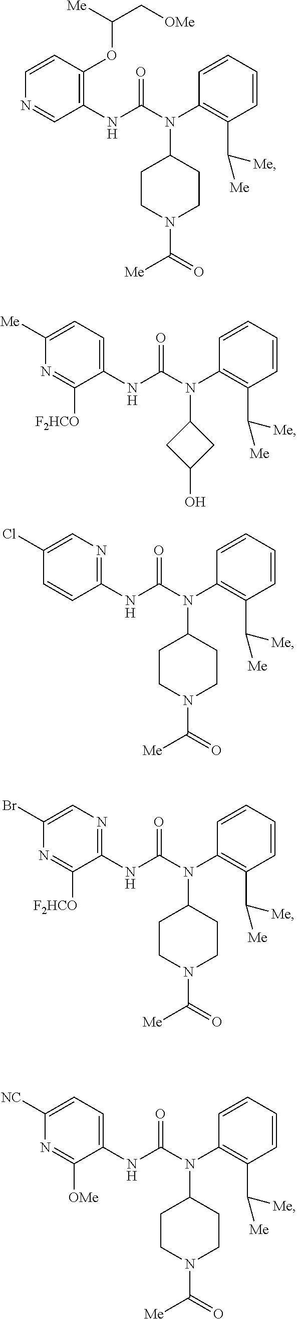 [SMILES: COCC(C)Oc1ccncc1NC(=O)N(c1ccccc1C(C)C)C1CCN(C(C)=O)CC1 ; Cc1ccc(NC(=O)N(c2ccccc2C(C)C)C2CC(O)C2)c(OC(F)F)n1 ; CC(=O)N1CCC(N(C(=O)Nc2ccc(Cl)cn2)c2ccccc2C(C)C)CC1 ; CC(=O)N1CCC(N(C(=O)Nc2ncc(Br)nc2OC(F)F)c2ccccc2C(C)C)CC1 ; COc1nc(C#N)ccc1NC(=O)N(c1ccccc1C(C)C)C1CCN(C(C)=O)CC1]

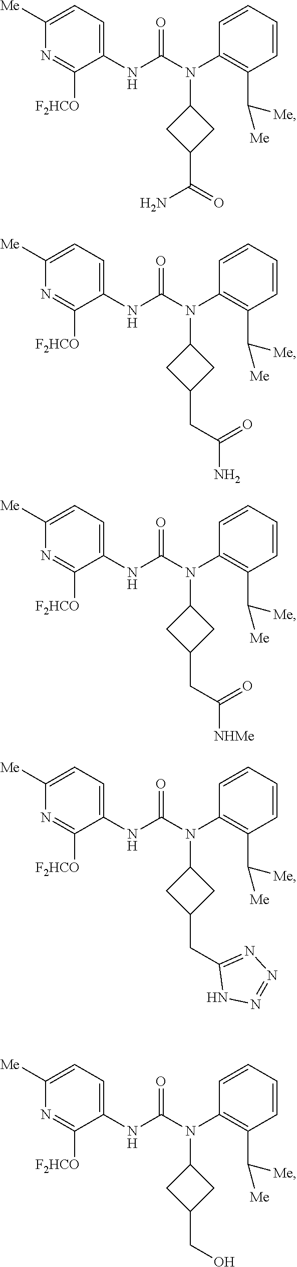

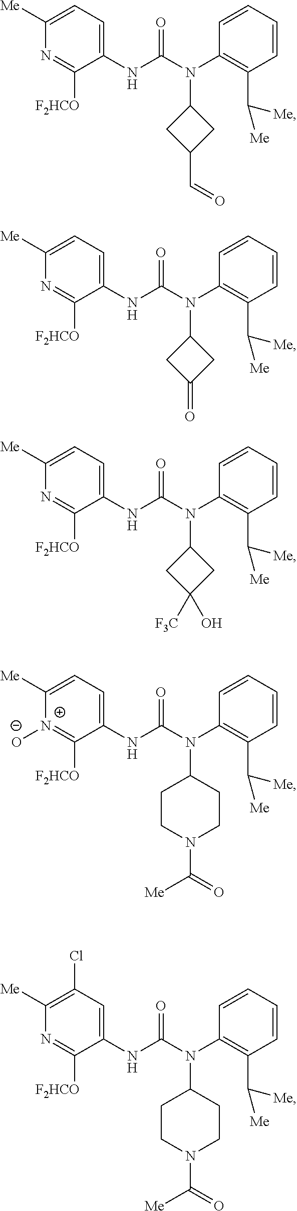

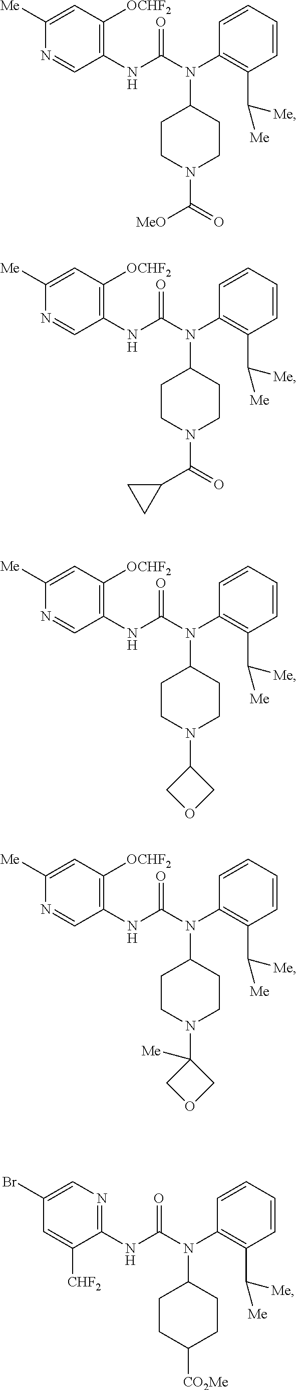 [SMILES: COC(=O)N1CCC(N(C(=O)Nc2cnc(C)cc2OC(F)F)c2ccccc2C(C)C)CC1 ; Cc1cc(OC(F)F)c(NC(=O)N(c2ccccc2C(C)C)C2CCN(C(=O)C3CC3)CC2)cn1 ; Cc1cc(OC(F)F)c(NC(=O)N(c2ccccc2C(C)C)C2CCN(C3COC3)CC2)cn1 ; Cc1cc(OC(F)F)c(NC(=O)N(c2ccccc2C(C)C)C2CCN(C3(C)COC3)CC2)cn1 ; COC(=O)C1CCC(N(C(=O)Nc2ncc(Br)cc2C(F)F)c2ccccc2C(C)C)CC1]

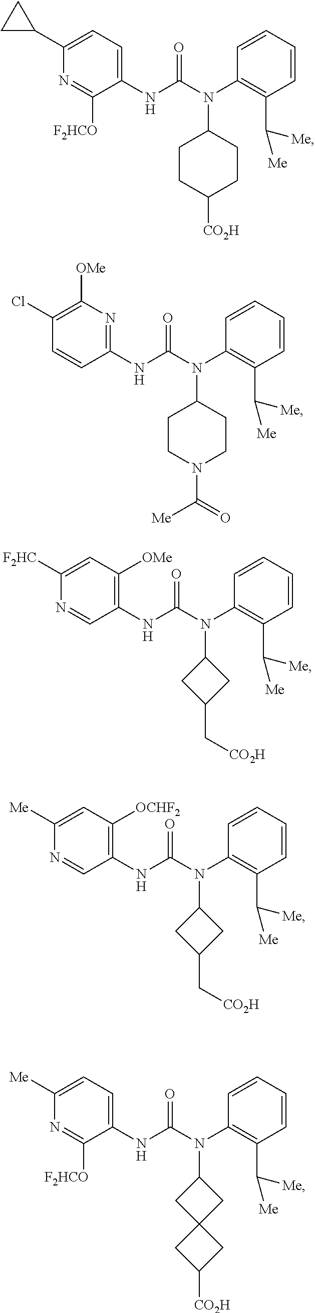

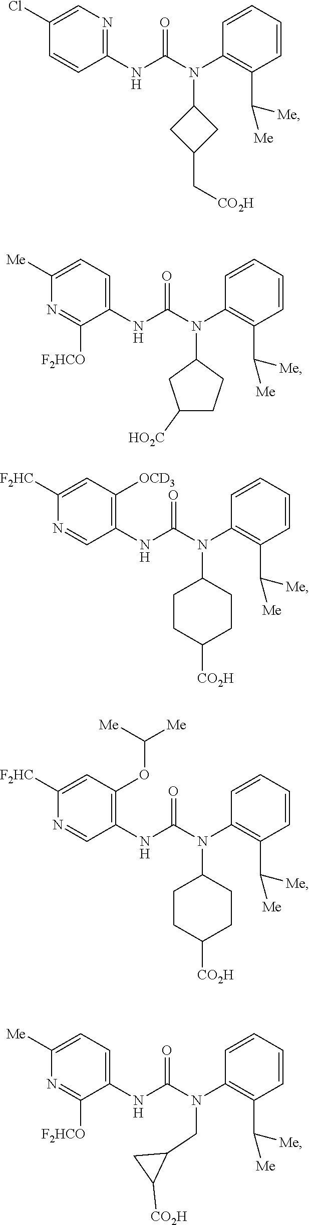

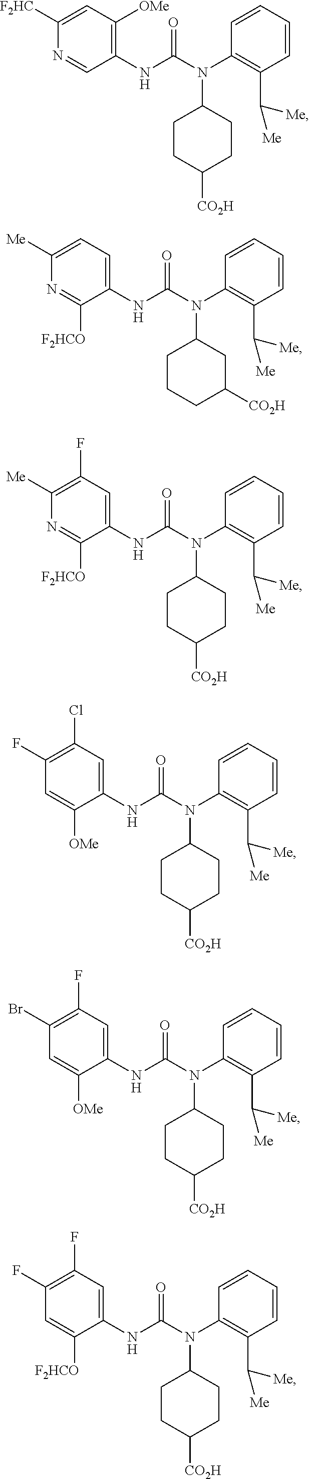 [SMILES: COc1cc(C(F)F)ncc1NC(=O)N(c1ccccc1C(C)C)C1CCC(C(=O)O)CC1 ; Cc1ccc(NC(=O)N(c2ccccc2C(C)C)C2CCCC(C(=O)O)C2)c(OC(F)F)n1 ; Cc1nc(OC(F)F)c(NC(=O)N(c2ccccc2C(C)C)C2CCC(C(=O)O)CC2)cc1F ; COc1cc(F)c(Cl)cc1NC(=O)N(c1ccccc1C(C)C)C1CCC(C(=O)O)CC1 ; COc1cc(Br)c(F)cc1NC(=O)N(c1ccccc1C(C)C)C1CCC(C(=O)O)CC1 ; CC(C)c1ccccc1N(C(=O)Nc1cc(F)c(F)cc1OC(F)F)C1CCC(C(=O)O)CC1]

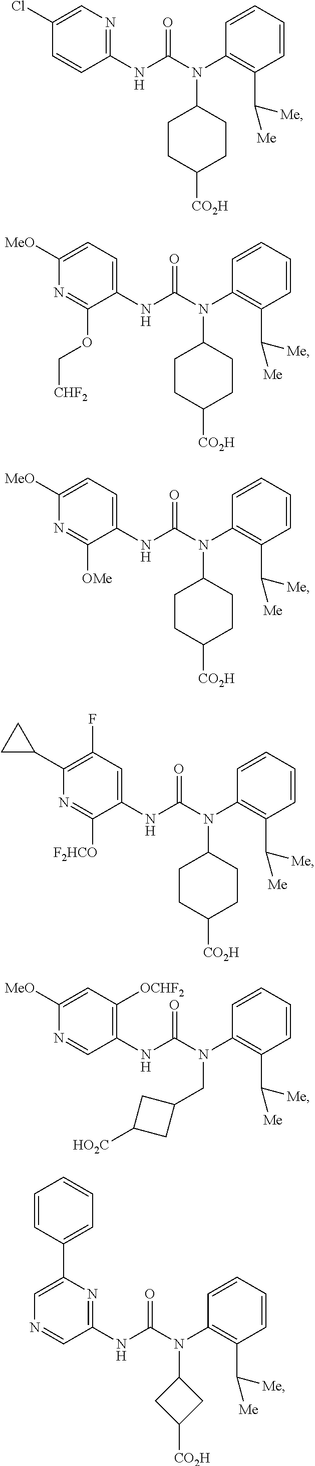 [SMILES: CC(C)c1ccccc1N(C(=O)Nc1ccc(Cl)cn1)C1CCC(C(=O)O)CC1 ; COc1ccc(NC(=O)N(c2ccccc2C(C)C)C2CCC(C(=O)O)CC2)c(OCC(F)F)n1 ; COc1ccc(NC(=O)N(c2ccccc2C(C)C)C2CCC(C(=O)O)CC2)c(OC)n1 ; CC(C)c1ccccc1N(C(=O)Nc1cc(F)c(C2CC2)nc1OC(F)F)C1CCC(C(=O)O)CC1 ; COc1cc(OC(F)F)c(NC(=O)N(CC2CC(C(=O)O)C2)c2ccccc2C(C)C)cn1 ; CC(C)c1ccccc1N(C(=O)Nc1cncc(-c2ccccc2)n1)C1CC(C(=O)O)C1]

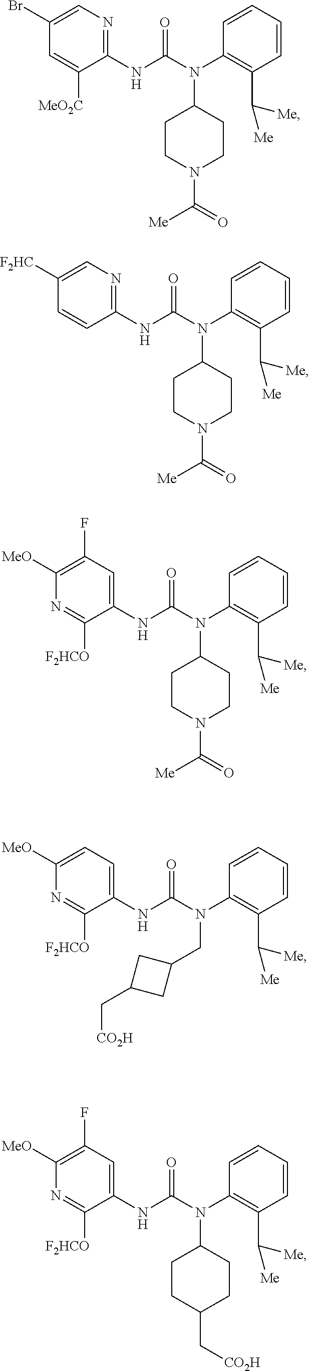 [SMILES: COC(=O)c1cc(Br)cnc1NC(=O)N(c1ccccc1C(C)C)C1CCN(C(C)=O)CC1 ; CC(=O)N1CCC(N(C(=O)Nc2ccc(C(F)F)cn2)c2ccccc2C(C)C)CC1 ; COc1nc(OC(F)F)c(NC(=O)N(c2ccccc2C(C)C)C2CCN(C(C)=O)CC2)cc1F ; COc1ccc(NC(=O)N(CC2CC(CC(=O)O)C2)c2ccccc2C(C)C)c(OC(F)F)n1 ; COc1nc(OC(F)F)c(NC(=O)N(c2ccccc2C(C)C)C2CCC(CC(=O)O)CC2)cc1F]

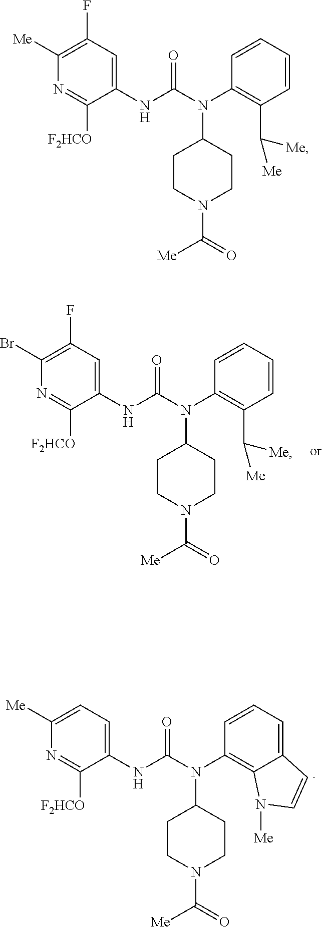 [SMILES: CC(=O)N1CCC(N(C(=O)Nc2cc(F)c(C)nc2OC(F)F)c2ccccc2C(C)C)CC1 ; CC(=O)N1CCC(N(C(=O)Nc2cc(F)c(Br)nc2OC(F)F)c2ccccc2C(C)C)CC1 ; "or" ; CC(=O)N1CCC(N(C(=O)Nc2ccc(C)nc2OC(F)F)c2cccc3ccn(C)c23)CC1]

Embodiment P41. A pharmaceutical composition comprising the compound of one of embodiments P1 to P40, or a pharmaceutically acceptable salt or solvate thereof, and a pharmaceutically acceptable excipient.

Embodiment P42. A method of treating a neurodegenerative disorder in a subject in need thereof, said method comprising administering to the subject in need thereof a therapeutically effective amount of a compound of one of embodiments P1 to P40, or a pharmaceutically acceptable salt or solvate thereof.

Embodiment P43. A method of treating an inflammatory disease in a subject in need thereof, said method comprising administering to the subject in need thereof a therapeutically effective amount of a compound of one of embodiments P1 to P40, or a pharmaceutically acceptable salt or solvate thereof.

Embodiment P44. The method of embodiment P43, wherein the inflammatory disease is encephalitis.

Embodiment P45. The method of embodiment P44, wherein the encephalitis is post-hemorrhagic encephalitis.

Embodiment P46. A method of treating a demyelinating disease in a subject in need thereof, said method comprising administering to the subject in need thereof a therapeutically effective amount of a compound of one of embodiments P1 to P40, or a pharmaceutically acceptable salt or solvate thereof.

Embodiment P47. The method of embodiment P46, wherein the demyelinating disease is a demyelinating disease of the central nervous system.

Embodiment P48. The method of embodiment P47, wherein the demyelinating disease is multiple sclerosis.

Embodiment P49. The method of embodiment P46, wherein the demyelinating disease is a demyelinating disease of the peripheral nervous system.

Embodiment P50. A method of treating a fibrotic disease in a subject in need thereof, said method comprising administering to the subject in need thereof a therapeutically effective amount of a compound of one of embodiments P1 to P40, or a pharmaceutically acceptable salt or solvate thereof.

Embodiment P51. The method of embodiment P50, wherein the fibrotic disease is pulmonary fibrosis, skin fibrosis, liver fibrosis, or ocular fibrosis.

Embodiment P52. The method of embodiment P50, wherein the fibrotic disease is idiopathic pulmonary fibrosis, scleroderma, nonalcoholic steatohepatitis, or ocular fibrosis.

Embodiment P53. A method of treating cancer in a subject in need thereof, said method comprising administering to the subject in need thereof a therapeutically effective amount of a compound of one of embodiments P1 to P40, or a pharmaceutically acceptable salt or solvate thereof.

Embodiment P54. The method of embodiment P53, wherein the cancer is brain cancer.

Embodiment P55. The method of embodiment P54, wherein the cancer is glioblastoma.

Embodiment P56. A method of modulating LPAR1 activity in a subject, said method comprising administering to the subject a compound of one of embodiments P1 to P40, or a pharmaceutically acceptable salt or solvate thereof.

### VI. Additional Embodiments

Embodiment 1. A compound, or a pharmaceutically acceptable salt or solvate thereof, having the formula:

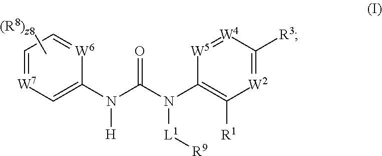

wherein
L¹ is a bond or substituted or unsubstituted C₁-C₅ alkylene;
R¹ is unsubstituted C₂-C₅ alkyl;
W² is N or C(R²);
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —N^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶);
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R⁹ is substituted or unsubstituted cycloalkyl or substituted or unsubstituted heterocycloalkyl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, and R^{8D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, and X⁸ are independently —F, —Cl, —Br, or —I;
n2, n3, n4, n5, n6, n7, and n8 are independently an integer from 0 to 4;
m2, m3, m4, m5, m6, m7, m8, v2, v3, v4, v5, v6, v7, and v8 are independently 1 or 2; and
z8 is an integer from 0 to 3.

Embodiment 2. The compound of embodiment 1, having the formula:

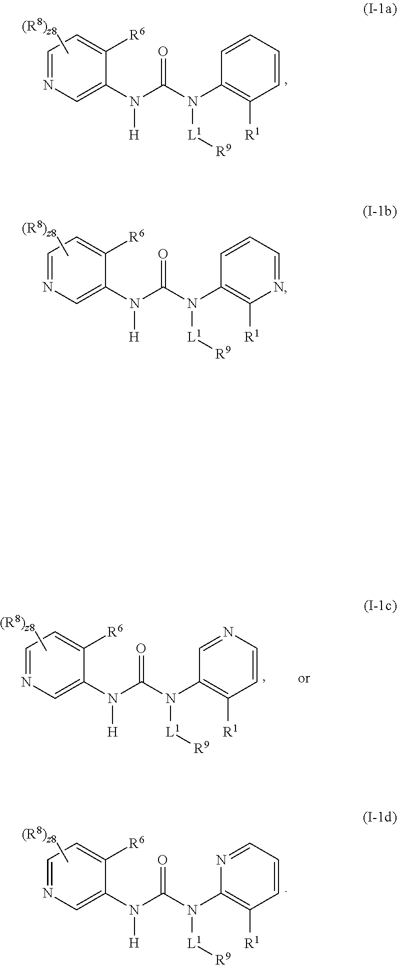

Embodiment 3. The compound of embodiment 1, having the formula:

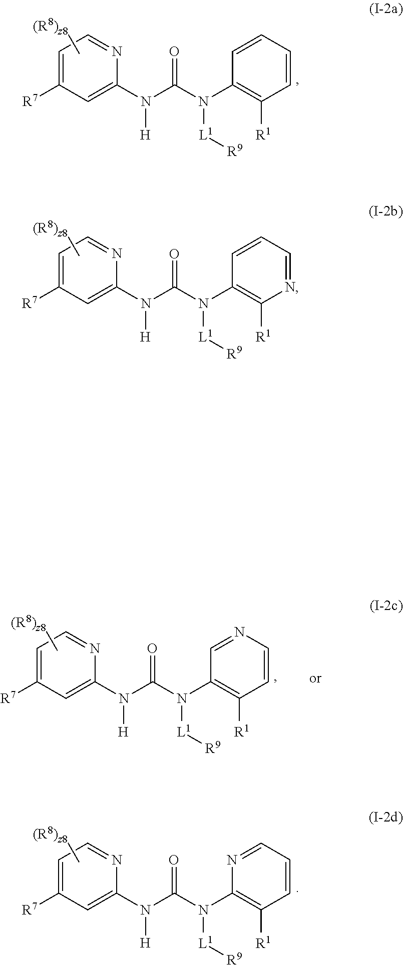

Embodiment 4. The compound of embodiment 1, having the formula:

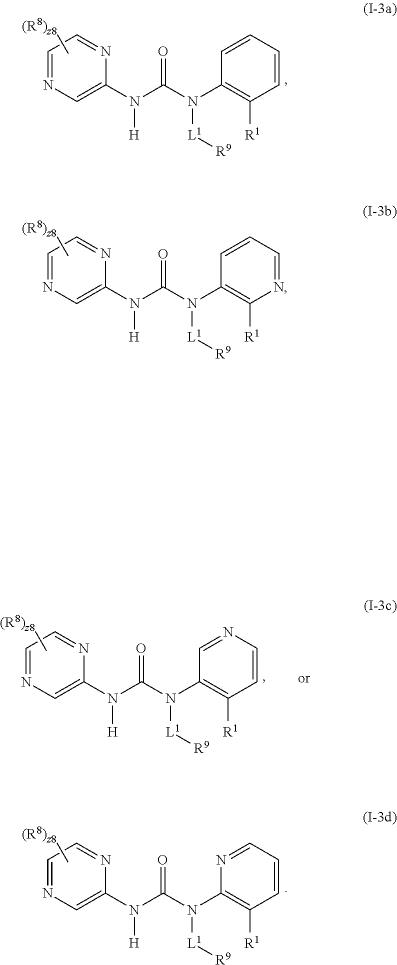

Embodiment 5. The compound of embodiment 1, having the formula:

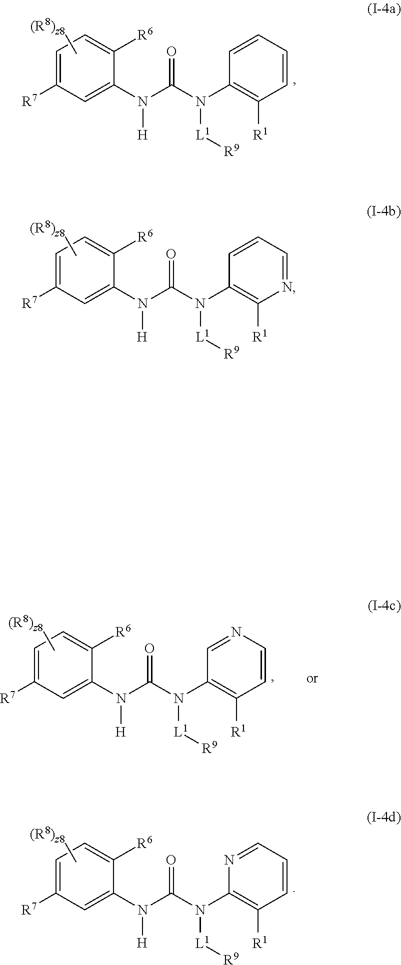

Embodiment 6. The compound of one of embodiments 1 to 5, wherein R¹ is unsubstituted C₃ alkyl.

Embodiment 7. The compound of one of embodiments 1 to 5, wherein R¹ is isopropyl.

Embodiment 8. A compound, or a pharmaceutically acceptable salt or solvate thereof, having the formula:

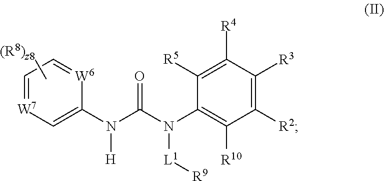

wherein
L¹ is a bond or substituted or unsubstituted C₁-C₅ alkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶);
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —N^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —N^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R⁹ is substituted or unsubstituted cycloalkyl or substituted or unsubstituted heterocycloalkyl;
R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, and X¹⁰ are independently —F, —Cl, —Br, or —I;
n2, n3, n4, n5, n6, n7, n8, and n10 are independently an integer from 0 to 4;
m2, m3, m4, m5, m6, m7, m8, m10, v2, v3, v4, v5, v6, v7, v8, and v10 are independently 1 or 2; and
z8 is an integer from 0 to 3;
wherein at least one of W⁶ or W⁷ is N;
wherein if W⁶ is C(R⁶) or W⁷ is C(R⁷), then R¹⁰ is not hydrogen;
wherein if W⁶ and W⁷ are both N, then R³ is not —S(O)₂CH₃; and
wherein if W⁶ is CH and W⁷ is N, then -L¹-R⁹ is not

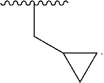 [SMILES: *CC1CC1]

Embodiment 9. The compound of embodiment 8, having the formula:

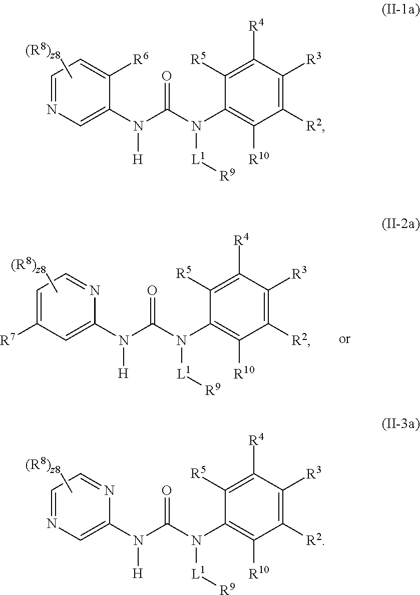

Embodiment 10. The compound of embodiment 8, having the formula:

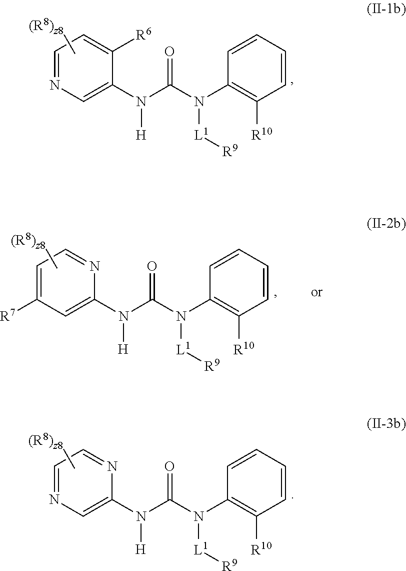

Embodiment 11. The compound of one of embodiments 8 to 10, wherein R¹⁰ is hydrogen or unsubstituted C₁-C₆ alkyl.

Embodiment 12. The compound of one of embodiments 8 to 10, wherein R¹⁰ is isopropyl.

Embodiment 13. The compound of one of embodiments 1 to 12, wherein R⁶ is hydrogen, —OCHF₂, unsubstituted C₁-C₆ alkyl, or unsubstituted 2 to 6 membered heteroalkyl.

Embodiment 14. The compound of one of embodiments 1 to 12, wherein R⁶ is hydrogen, —OCHF₂, unsubstituted methoxy, or unsubstituted isopropoxy.

Embodiment 15. The compound of one of embodiments 1 to 14, wherein R⁷ is hydrogen, —F, —Cl, —Br, or —OCHF₂.

Embodiment 16. The compound of one of embodiments 1 to 15, wherein R⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 17. The compound of one of embodiments 1 to 15, wherein R⁸ is independently halogen, —CF₃, —CHF₂, —CN, —OCHF₂, —C(O)R^{8C}, —C(O)OR^{8C}, —OR^{8D}, unsubstituted C₁-C₆ alkyl, unsubstituted 2 to 8 membered heteroalkyl, unsubstituted C₃-C₈ cycloalkyl, or unsubstituted phenyl.

Embodiment 18. The compound of embodiment 17, wherein R^{8C} is independently hydrogen or unsubstituted C₁-C₆ alkyl.

Embodiment 19. The compound of embodiment 17, wherein R^{8D} is independently unsubstituted C₁-C₆ alkyl.

Embodiment 20. The compound of one of embodiments 1 to 15, wherein R⁸ is independently —F, —Cl, —Br, —CF₃, —CHF₂, —CN, —C(O)H, —C(O)OCH₃, —OCHF₂, —OCH₃, —OCH₂CH₃, —OCH₂CF₃, —OCH(CH₃)CH₂OCH₃, —OCH₂CHF₂, unsubstituted methyl, unsubstituted cyclopropyl, or unsubstituted phenyl.

Embodiment 21. The compound of one of embodiments 1 to 15, wherein two R⁸ substituents are joined to form an unsubstituted C₅ cycloalkyl.

Embodiment 22. The compound of one of embodiments 1 to 21, wherein L¹ is a bond or unsubstituted C₁-C₅ alkylene.

Embodiment 23. The compound of one of embodiments 1 to 21, wherein L¹ is a bond.

Embodiment 24. The compound of one of embodiments 1 to 21, wherein L¹ is unsubstituted methylene.

Embodiment 25. The compound of one of embodiments 1 to 24, wherein R⁹ is an R¹¹-substituted or unsubstituted cycloalkyl or R¹¹-substituted or unsubstituted heterocycloalkyl; R¹¹ is independently oxo, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR^{11C}NR^{11A}R^{11B}, —ONR^{11A}R^{11B}, —NHC(O)NR^{11C}NR^{11A}R^{11B}, —NHC(O)NR^{11A}R^{11B}, —N(O)ₘ₁₁, —NR^{11A}R^{11B}, —C(O)R^{11C}, —C(O)OR^{11C}, —C(O)NR^{11A}R^{11B}, —OR^{11D}, —SR^{11D}, —NR^{11A}SO₂R^{11D}, —NR^{11A}C(O)R^{11C}, —NR^{11A}C(O)OR^{11C}, —NR^{11A}OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{11A}, R^{11B}, R^{11C}, and R^{11D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl;
X¹¹ is independently —F, —Cl, —Br, or —I;
n11 is independently an integer from 0 to 4; and
m11 and v11 are independently 1 or 2.

Embodiment 26. The compound of embodiment 25, wherein R⁹ is an R¹¹-substituted or unsubstituted C₃-C₈ cycloalkyl or R¹¹-substituted or unsubstituted 3 to 8 membered heterocycloalkyl.

Embodiment 27. The compound of embodiment 25, wherein R⁹ is an R¹¹-substituted or unsubstituted spirocyclic cycloalkyl or R¹¹-substituted or unsubstituted spirocyclic heterocycloalkyl.

Embodiment 28. The compound of embodiment 25, wherein R⁹ is

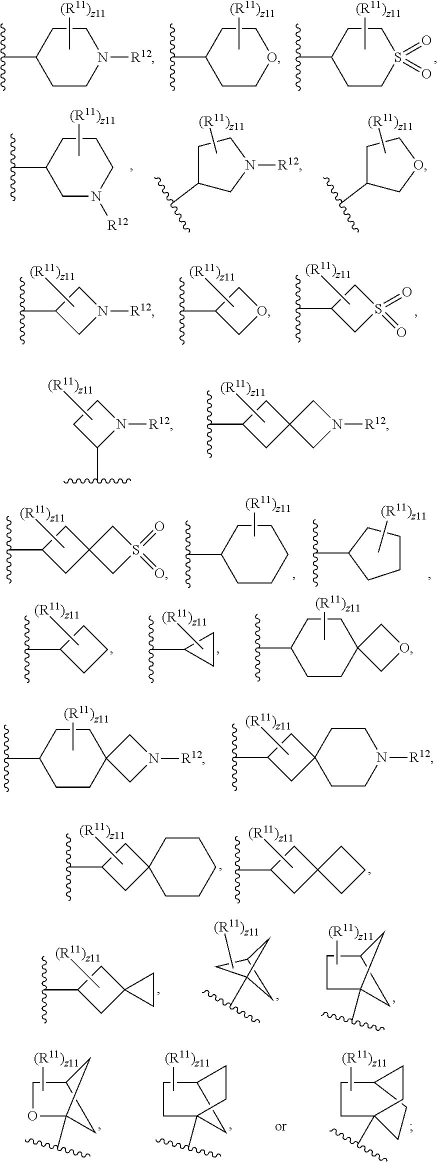

R¹² is hydrogen, halogen, —CX¹²₃, —CHX¹²₂, —CH₂X¹², —OCX¹²₃, —OCH₂X¹², —OCHX¹²₂, —SOₙ₁₂R^{12D}, —SOᵥ₁₂NR^{12A}R^{12B}, —C(O)R^{12C}, —C(O)OR^{12C}, —C(O)NR^{12A}R^{12B}, —OR^{12D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{12A}, R^{12B}, R^{12C}, and R^{12D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl;
X¹² is independently —F, —Cl, —Br, or —I;
n12 is independently an integer from 0 to 4;
v12 is independently 1 or 2; and
z11 is an integer from 0 to 13.

Embodiment 29. The compound of embodiment 28, wherein R⁹ is

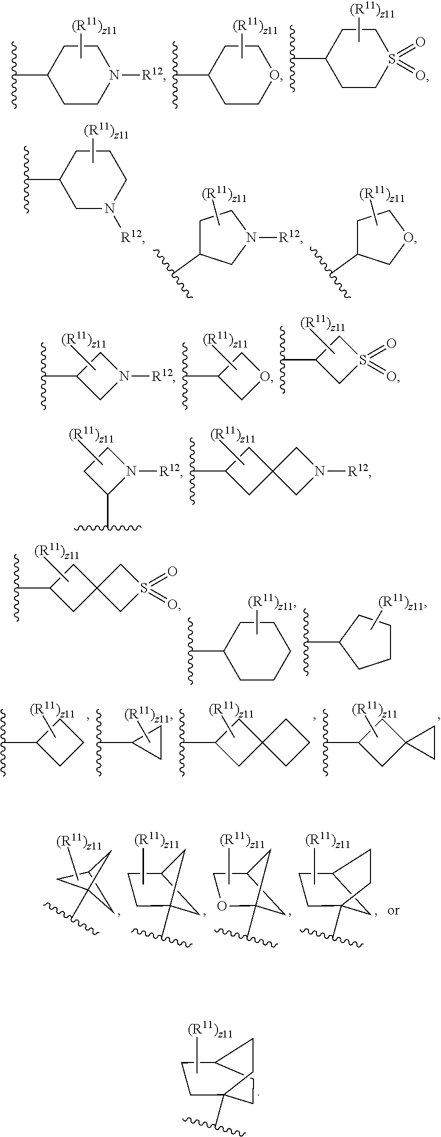

Embodiment 30. The compound of one of embodiments 25 to 29, wherein R¹¹ is independently oxo, halogen, —CX¹¹₃, —CHX¹¹₂, —CN, —SOₙ₁₁R^{11D}, —C(O)R^{11C}, —C(O)OR^{11C}, —C(O)NR^{11A}R^{11B}, —C(O)R^{11C}, —OR^{11D}, —NR^{11A}SO₂R^{11D}, —NR^{11A}C(O)R^{11C}, —NR^{11A}C(O)OR^{11C}, substituted or unsubstituted C₁-C₆ alkyl, substituted or unsubstituted 2 to 5 membered heteroalkyl, or substituted or unsubstituted 5 to 10 membered heteroaryl.

Embodiment 31. The compound of embodiment 30, wherein R^{11A} is independently hydrogen or unsubstituted C₁-C₆ alkyl; and R^{11B} is independently hydrogen.

Embodiment 32. The compound of embodiment 30, wherein R^{11C} is independently hydrogen or unsubstituted C₁-C₆ alkyl.

Embodiment 33. The compound of embodiment 30, wherein R^{11D} is independently hydrogen or unsubstituted C₁-C₆ alkyl.

Embodiment 34. The compound of one of embodiments 25 to 29, wherein two R¹¹ substituents are joined to form a substituted or unsubstituted cycloalkyl.

Embodiment 35. The compound of one of embodiments 28 to 29, wherein R¹² is hydrogen, —C(O)R^{12C}, —SOₙ₁₂R^{12D}, —SOᵥ₁₂NR^{12A}R^{12B}, —C(O)OR^{12C}, —C(O)NR^{12A}R^{12B}, unsubstituted C₁-C₆ alkyl, unsubstituted C₃-C₈ cycloalkyl, or unsubstituted 3 to 8 membered heterocycloalkyl.

Embodiment 36. The compound of embodiment 35, wherein R^{12A} is hydrogen, unsubstituted C₁-C₆ alkyl, or unsubstituted C₃-C₈ cycloalkyl; and R^{12B} is hydrogen.

Embodiment 37. The compound of embodiment 35, wherein R^{12C} is substituted or unsubstituted C₁-C₆ alkyl, substituted or unsubstituted 2 to 6 membered heteroalkyl, or substituted or unsubstituted C₃-C₈ cycloalkyl.

Embodiment 38. The compound of embodiment 35, wherein R^{12D} is unsubstituted C₁-C₆ alkyl.

Embodiment 39. The compound of one of embodiments 1 to 24, wherein R⁹ is

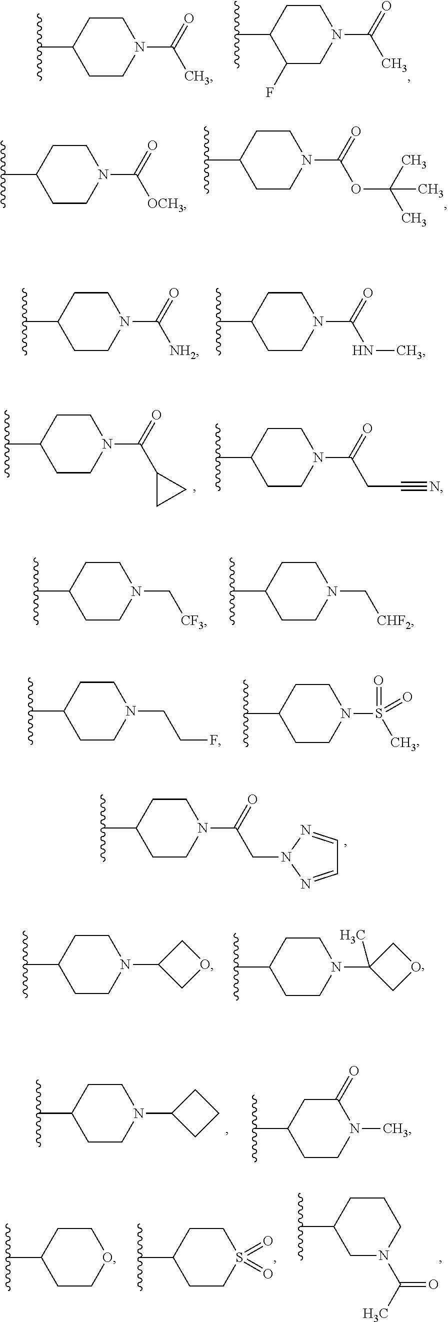

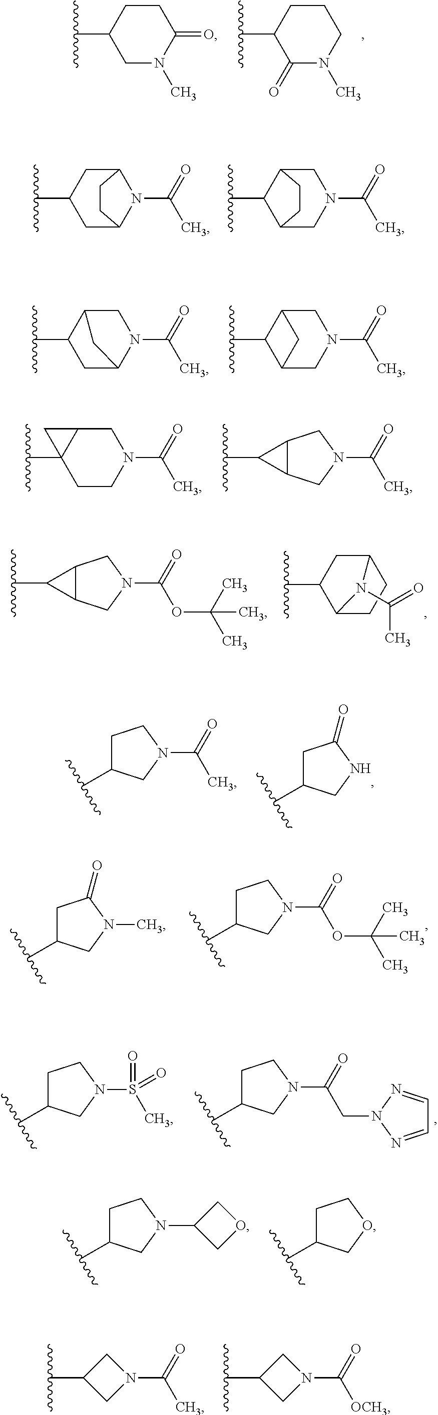

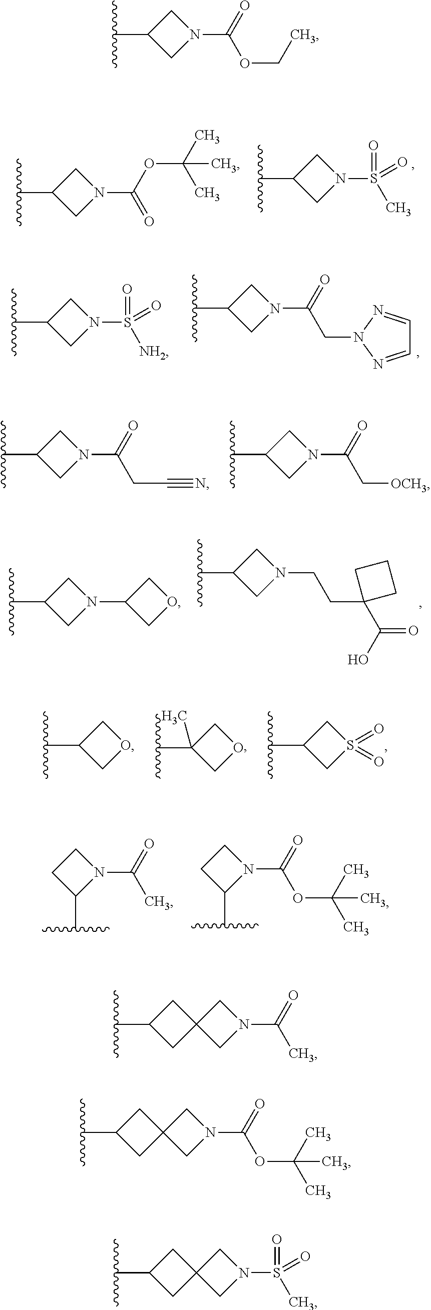

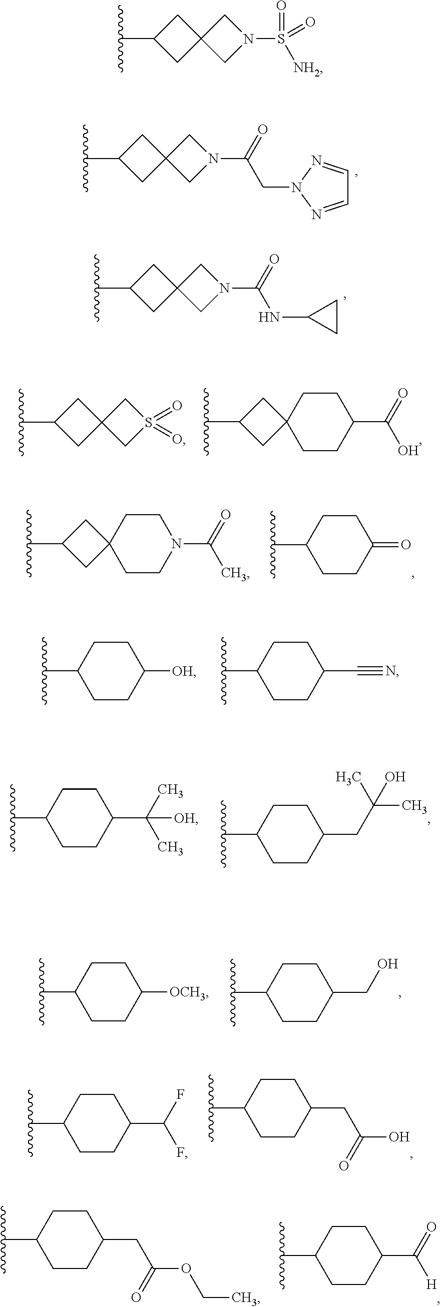

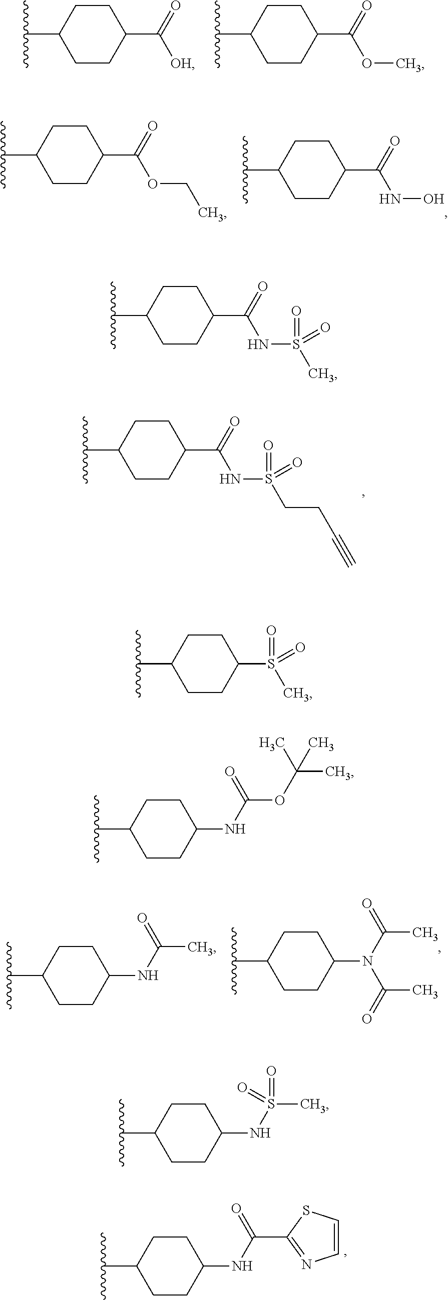

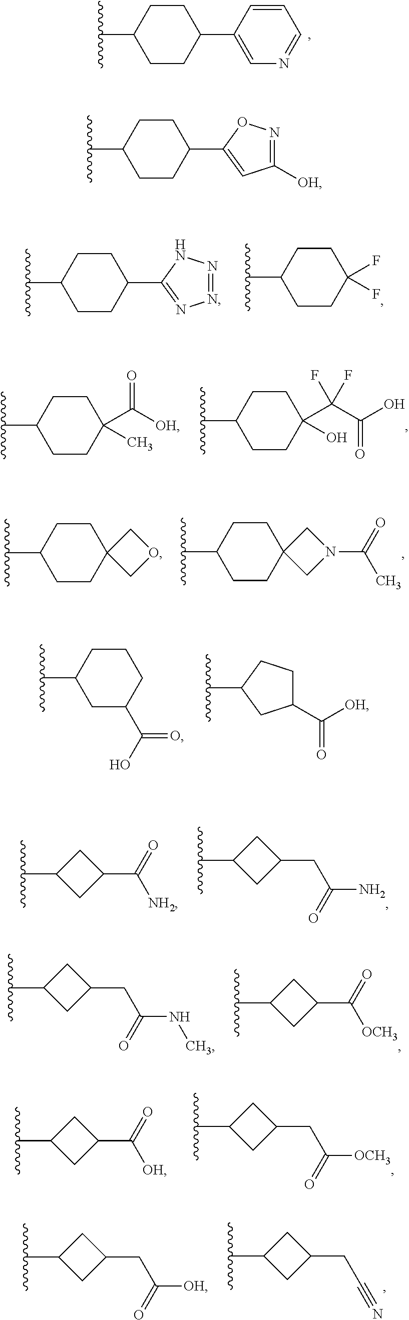

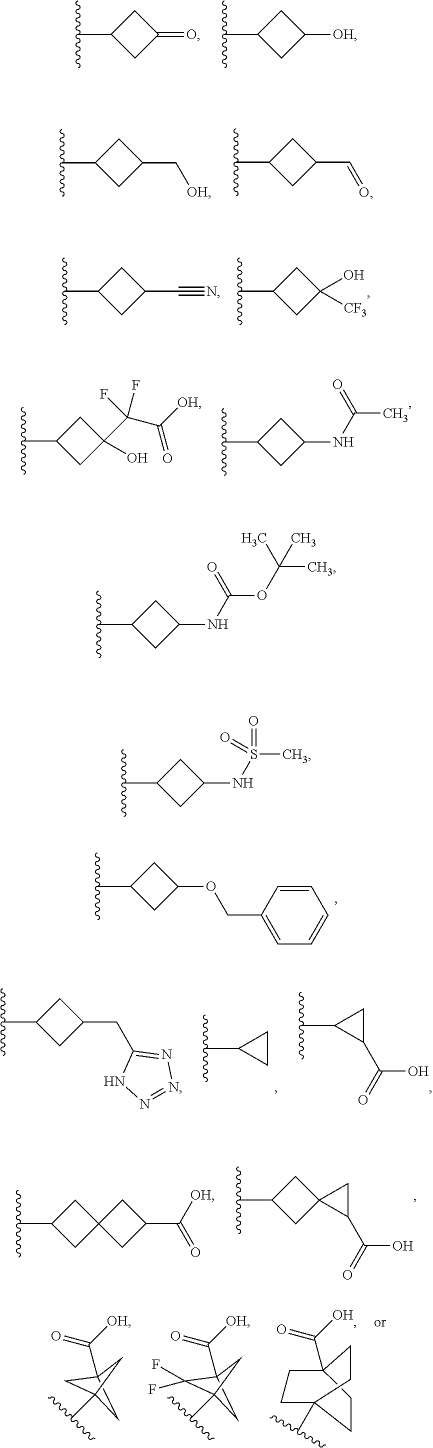

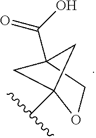 [SMILES: *C12CC(C(=O)O)(CO1)C2]

Embodiment 40. A compound, or a pharmaceutically acceptable salt or solvate thereof, having the formula:

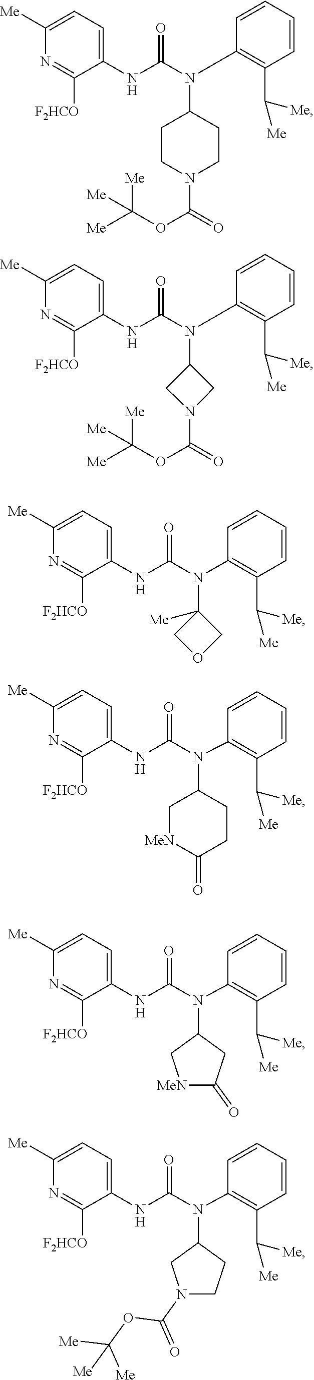

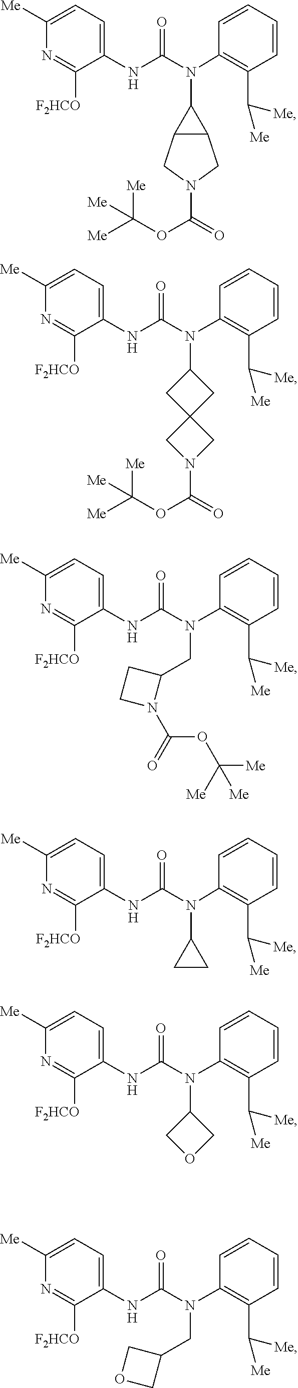

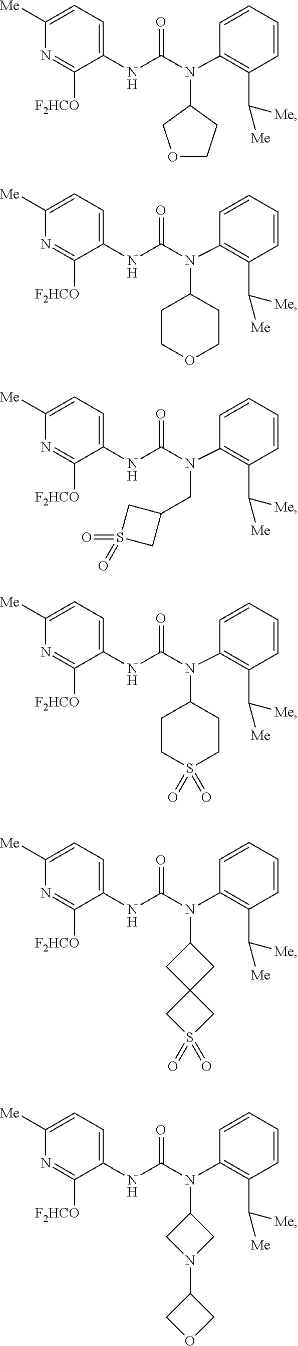

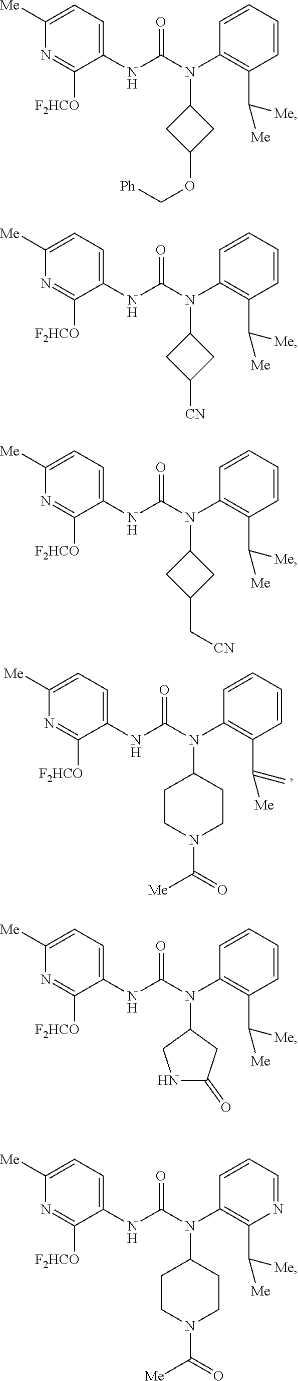

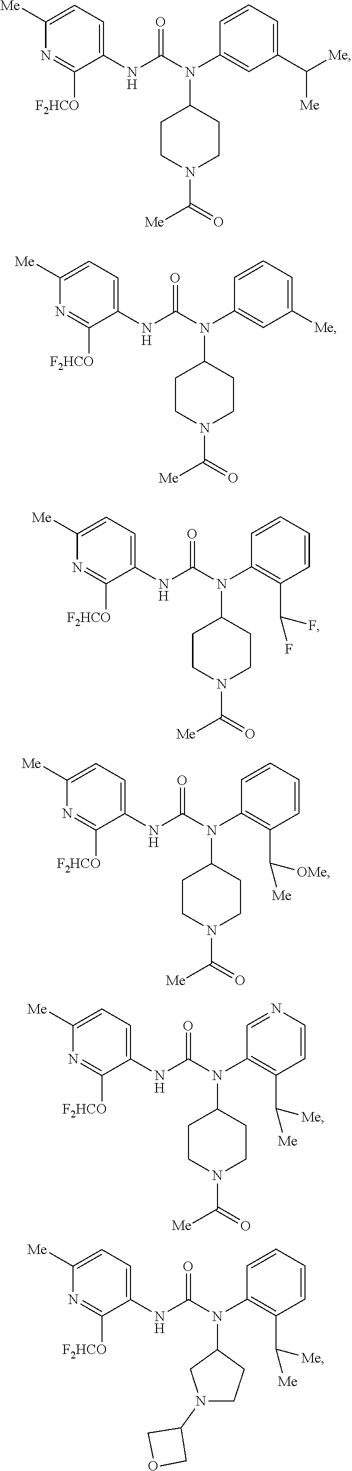

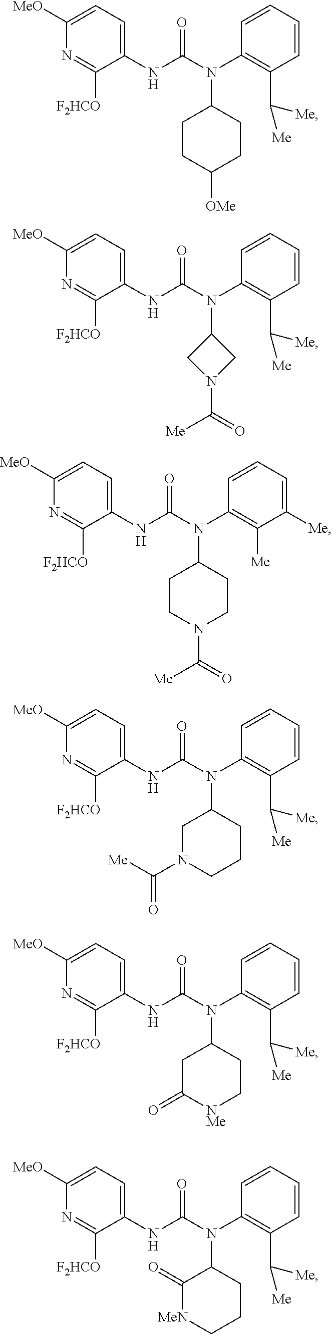

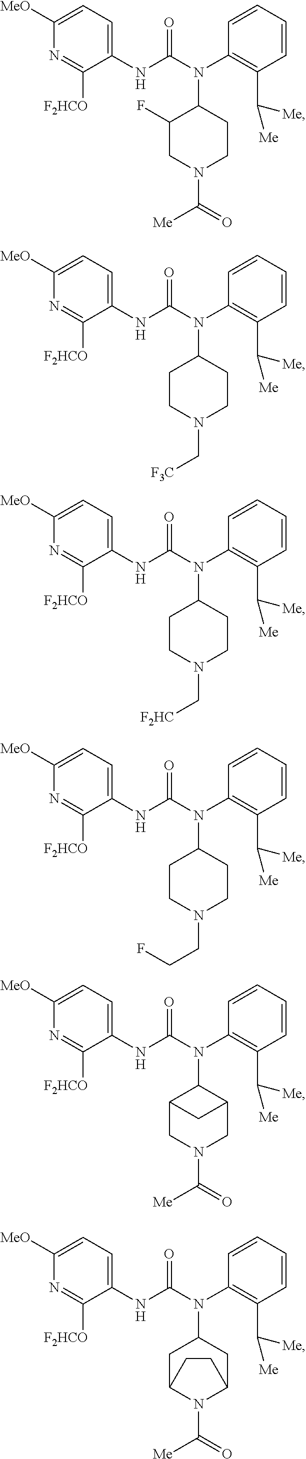

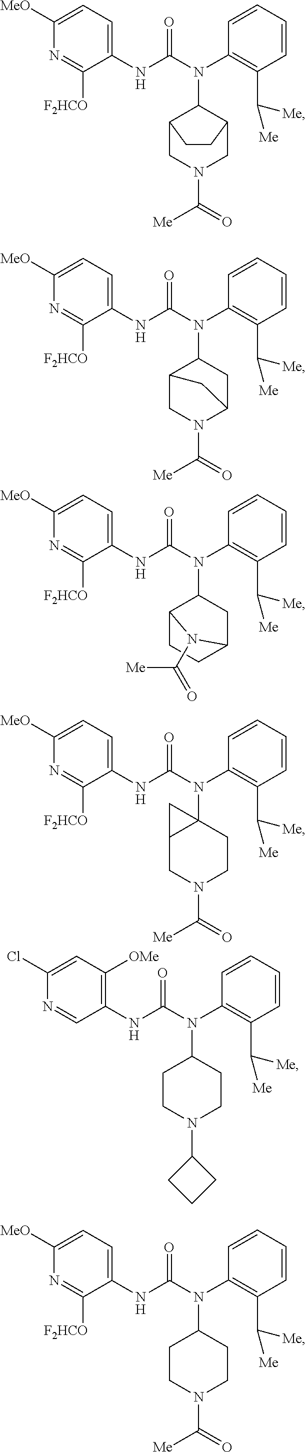

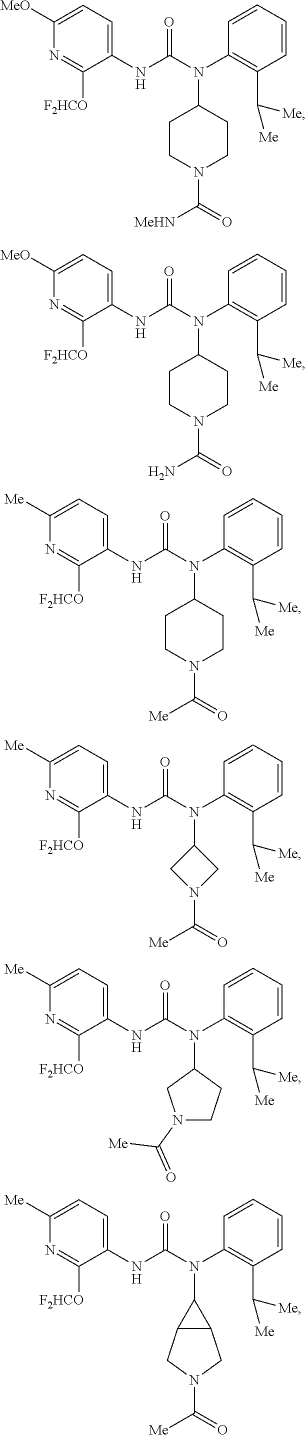

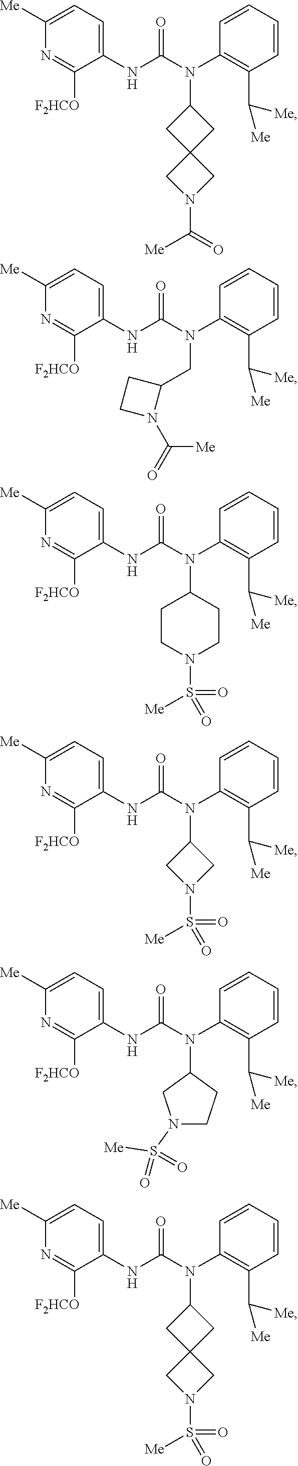

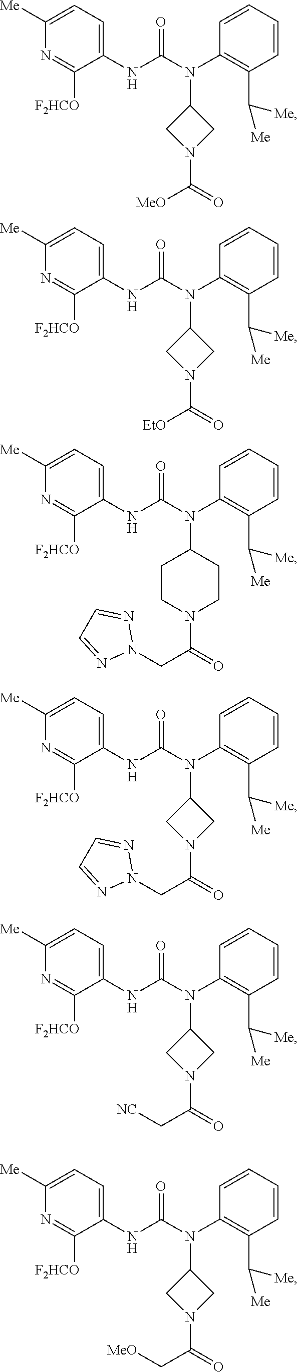

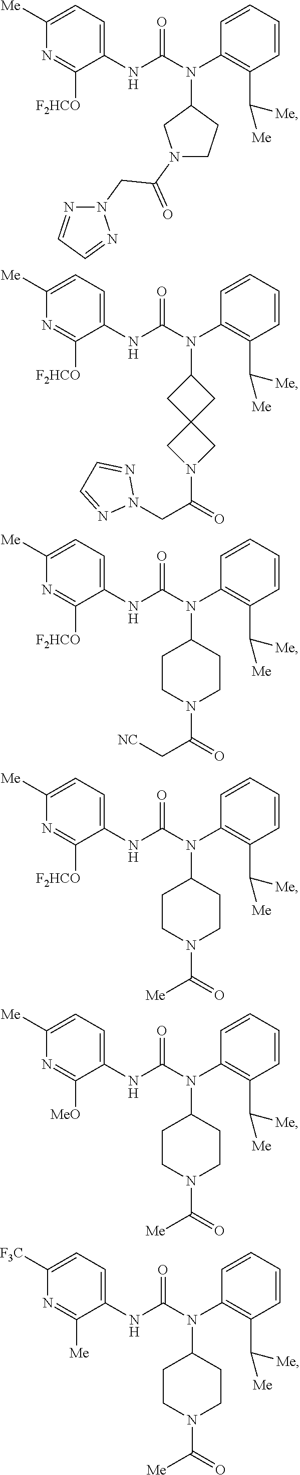

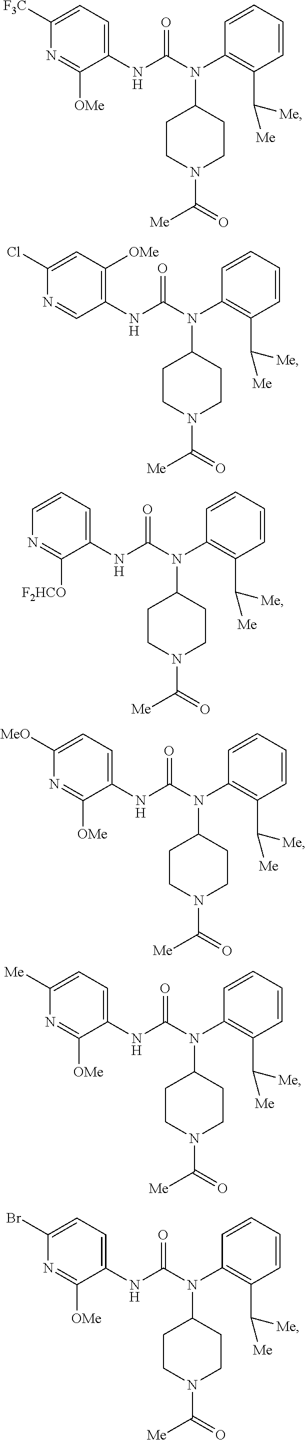

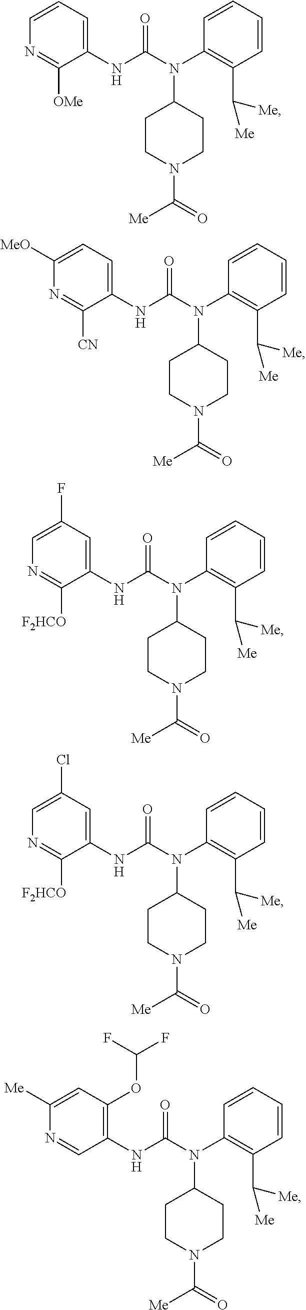

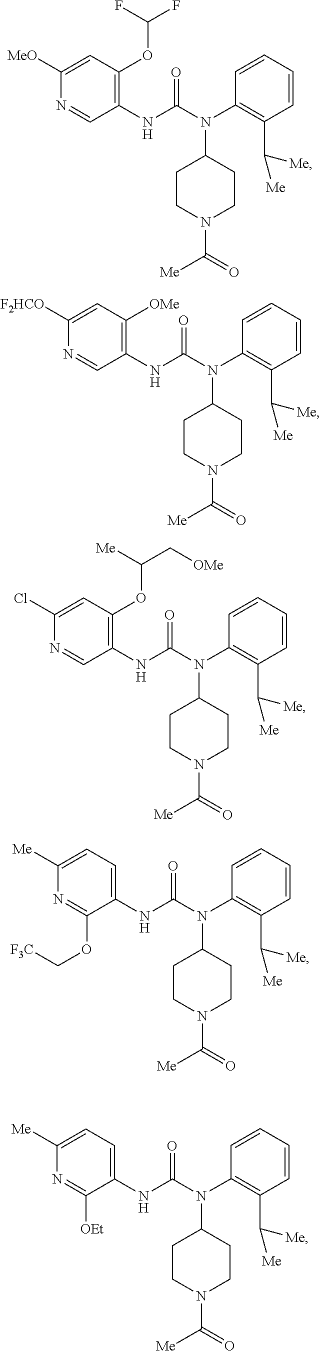

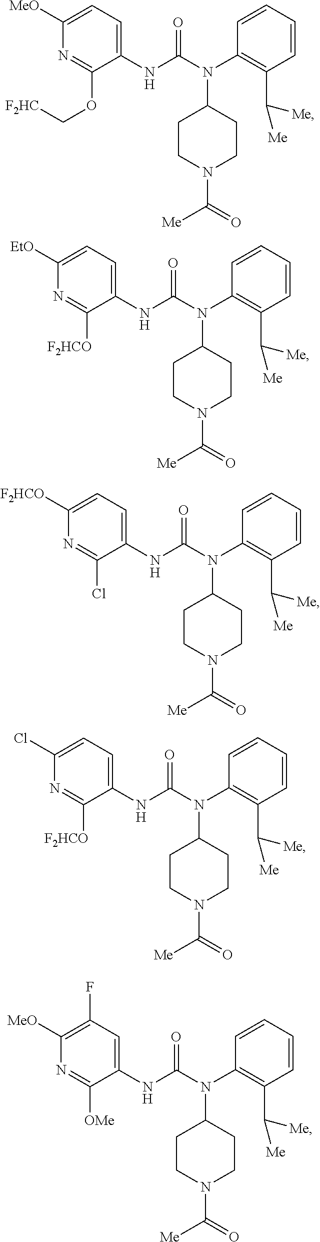

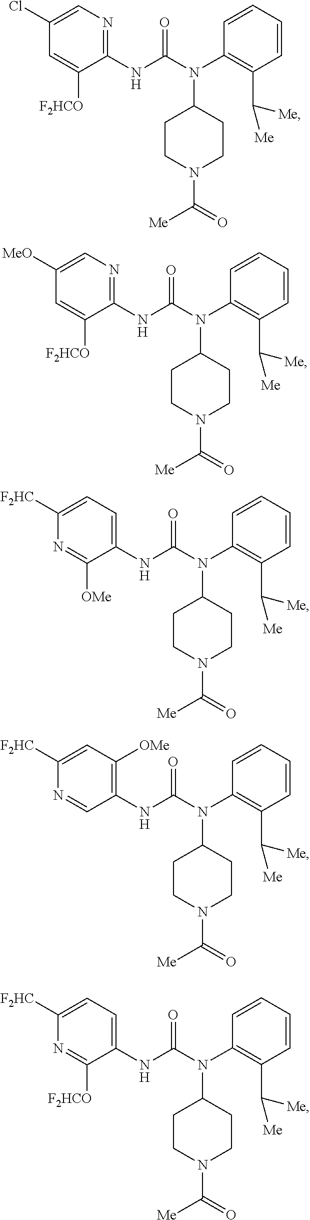

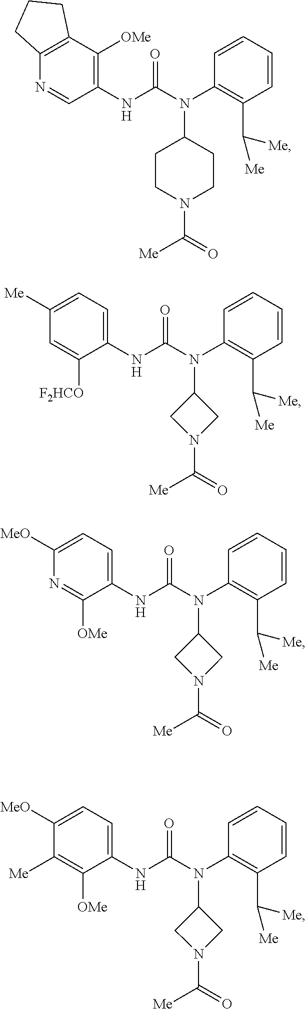 [SMILES: COc1c(NC(=O)N(c2ccccc2C(C)C)C2CCN(C(C)=O)CC2)cnc2c1CCC2 ; CC(=O)N1CC(N(C(=O)Nc2ccc(C)cc2OC(F)F)c2ccccc2C(C)C)C1 ; COc1ccc(NC(=O)N(c2ccccc2C(C)C)C2CN(C(C)=O)C2)c(OC)n1 ; COc1ccc(NC(=O)N(c2ccccc2C(C)C)C2CN(C(C)=O)C2)c(OC)c1C]

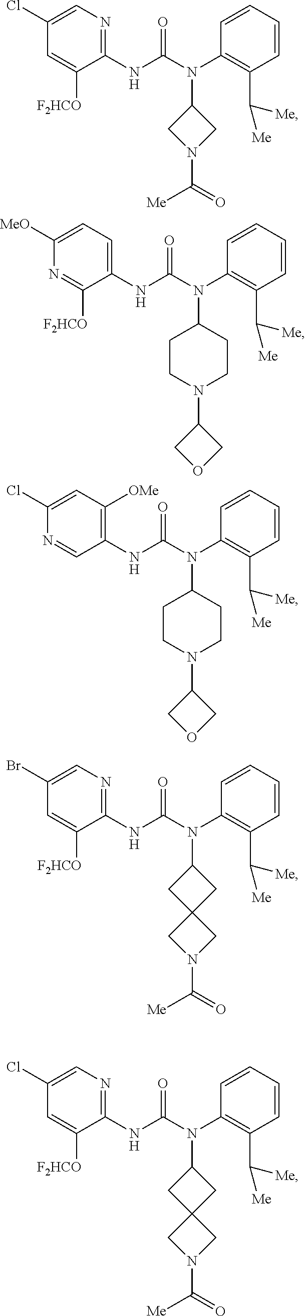

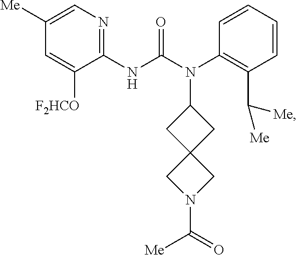 [SMILES: CC(=O)N1CC2(CC(N(C(=O)Nc3ncc(C)cc3OC(F)F)c3ccccc3C(C)C)C2)C1]

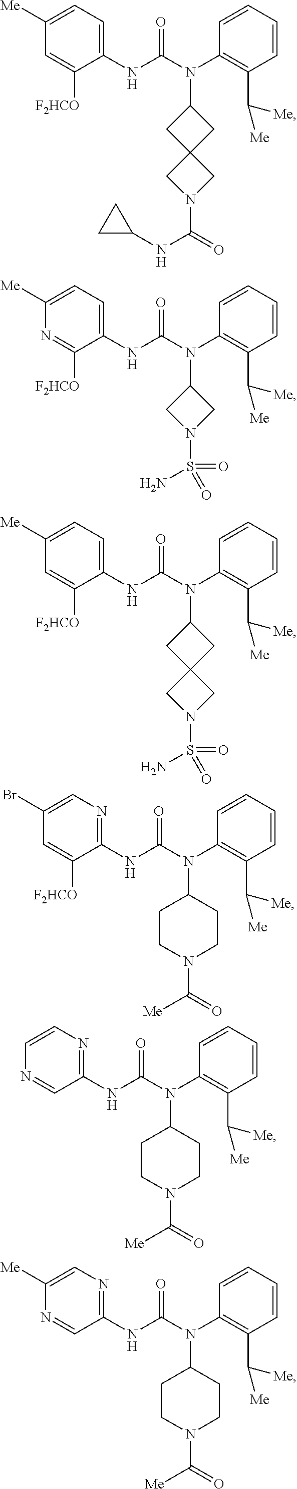

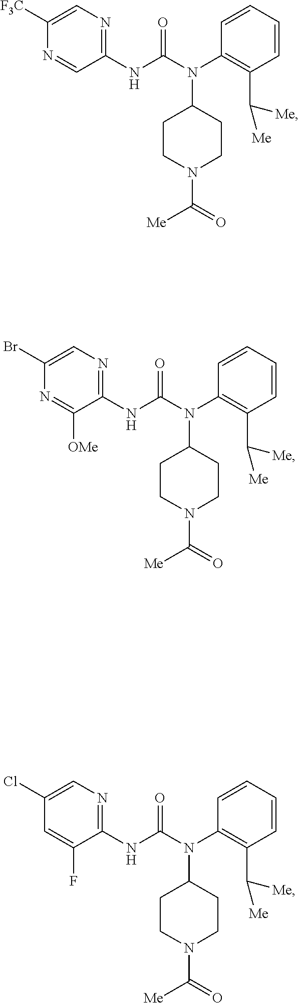 [SMILES: CC(=O)N1CCC(N(C(=O)Nc2cnc(C(F)(F)F)cn2)c2ccccc2C(C)C)CC1 ; COc1nc(Br)cnc1NC(=O)N(c1ccccc1C(C)C)C1CCN(C(C)=O)CC1 ; CC(=O)N1CCC(N(C(=O)Nc2ncc(Cl)cc2F)c2ccccc2C(C)C)CC1]

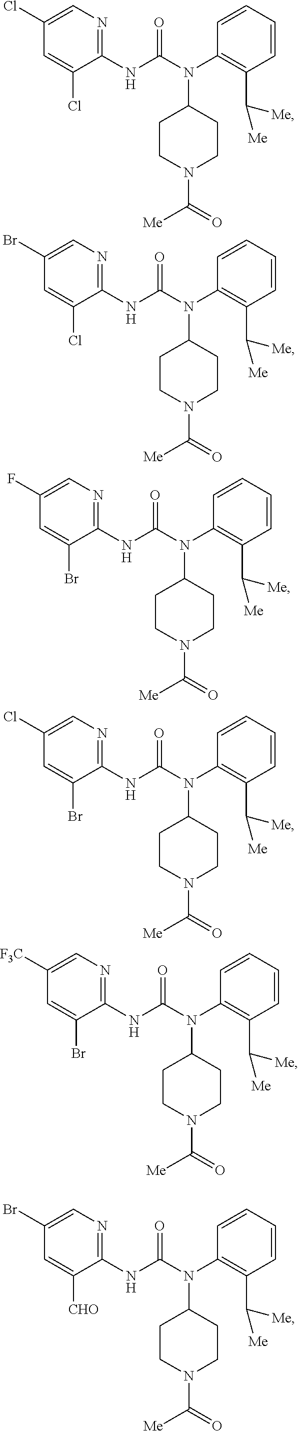

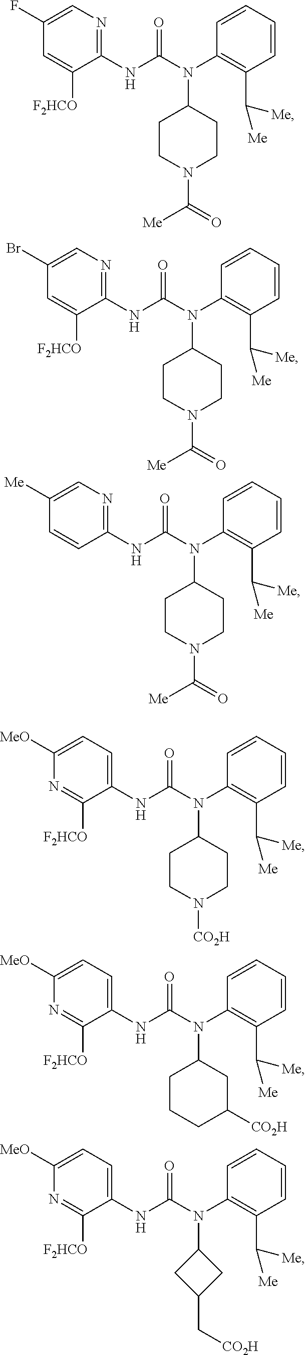

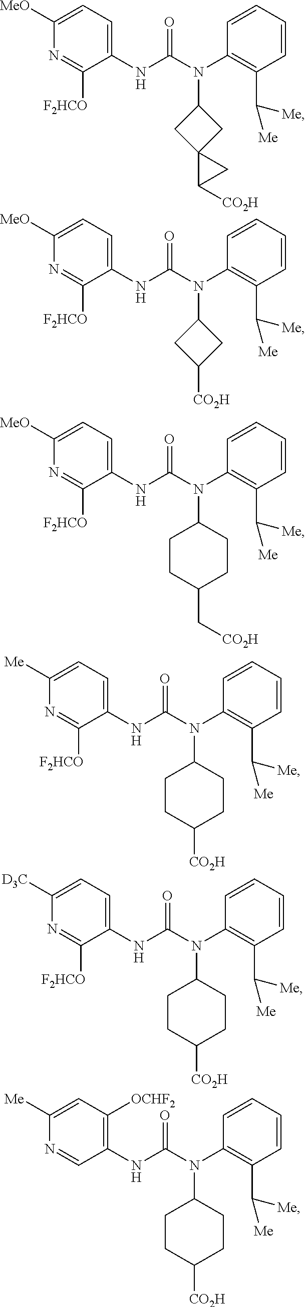

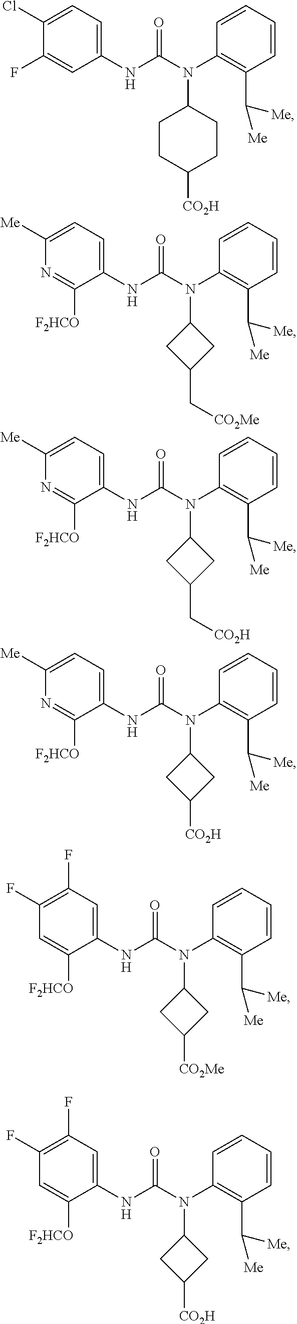

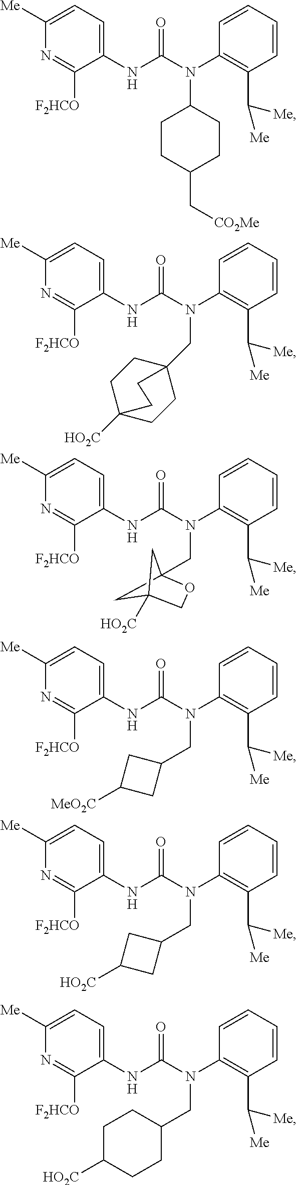

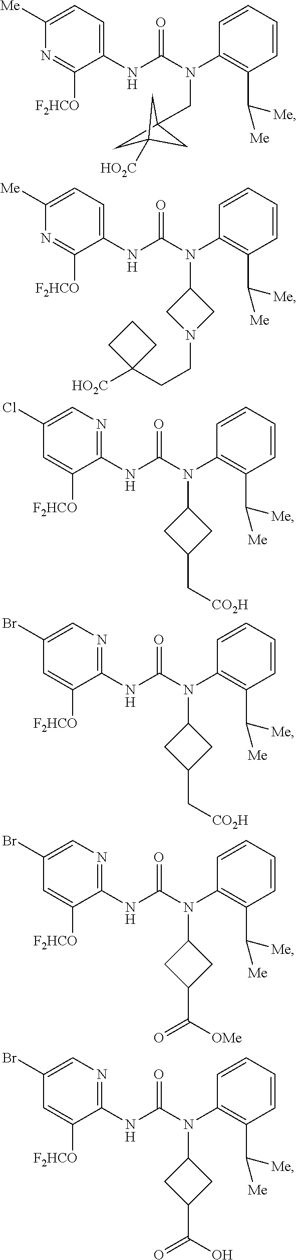

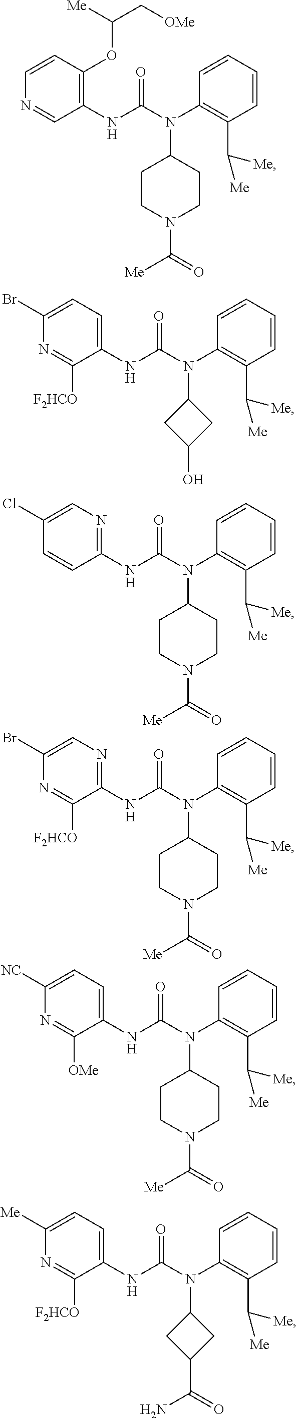

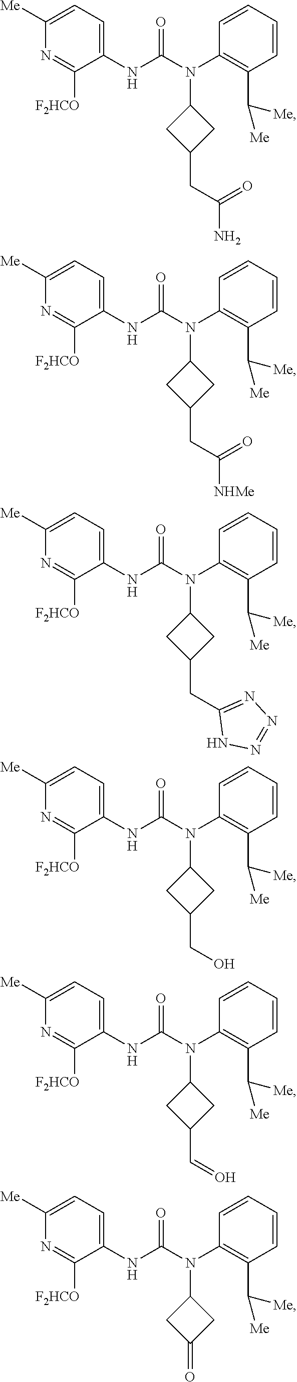

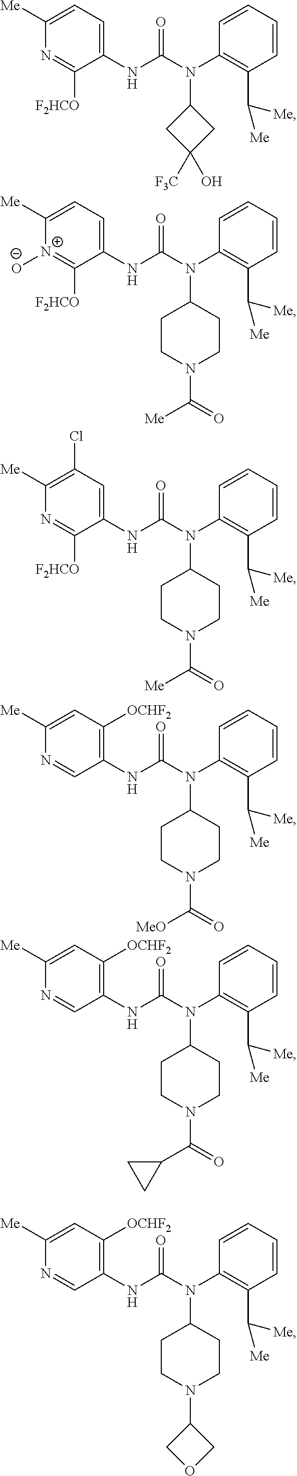

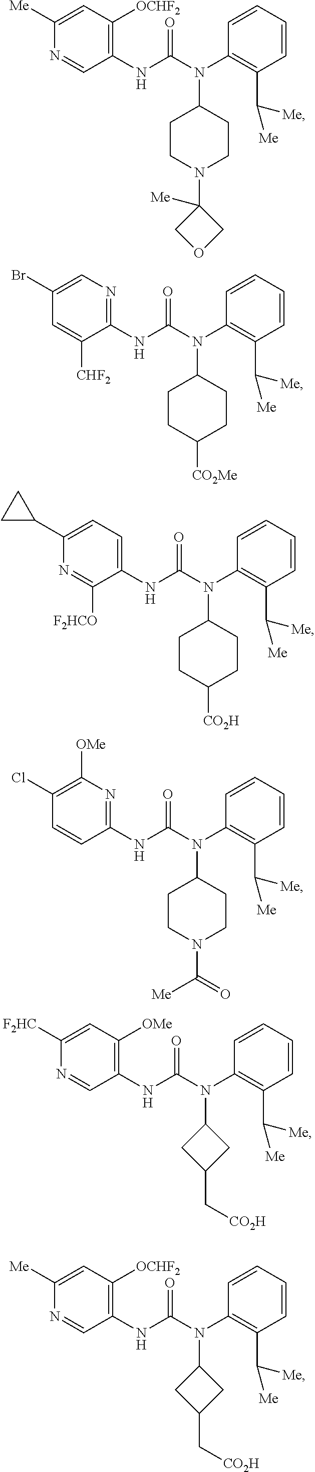

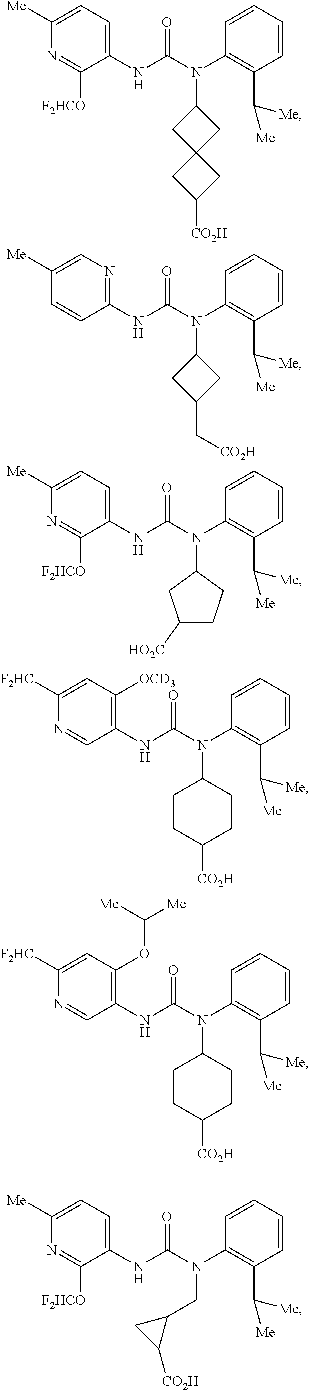

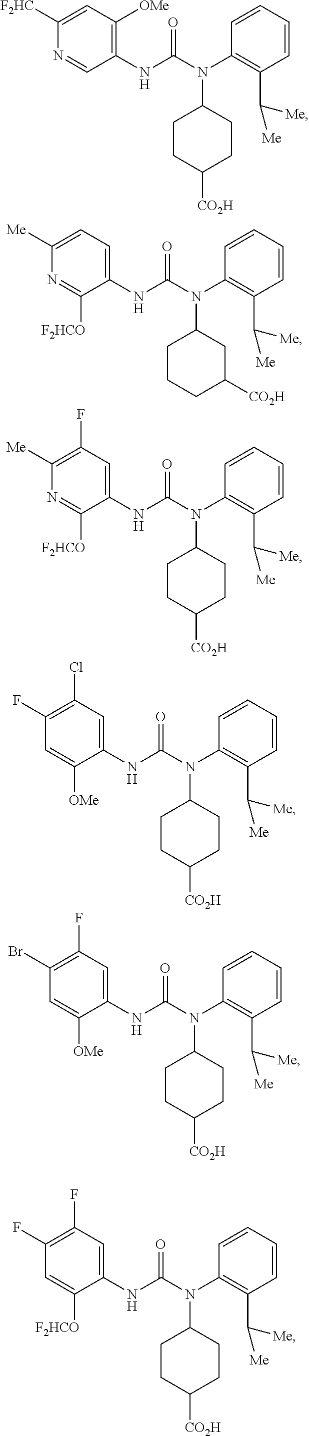 [SMILES: COc1cc(C(F)F)ncc1NC(=O)N(c1ccccc1C(C)C)C1CCC(C(=O)O)CC1 ; Cc1ccc(NC(=O)N(c2ccccc2C(C)C)C2CCCC(C(=O)O)C2)c(OC(F)F)n1 ; Cc1nc(OC(F)F)c(NC(=O)N(c2ccccc2C(C)C)C2CCC(C(=O)O)CC2)cc1F ; COc1cc(F)c(Cl)cc1NC(=O)N(c1ccccc1C(C)C)C1CCC(C(=O)O)CC1 ; COc1cc(Br)c(F)cc1NC(=O)N(c1ccccc1C(C)C)C1CCC(C(=O)O)CC1 ; CC(C)c1ccccc1N(C(=O)Nc1cc(F)c(F)cc1OC(F)F)C1CCC(C(=O)O)CC1]

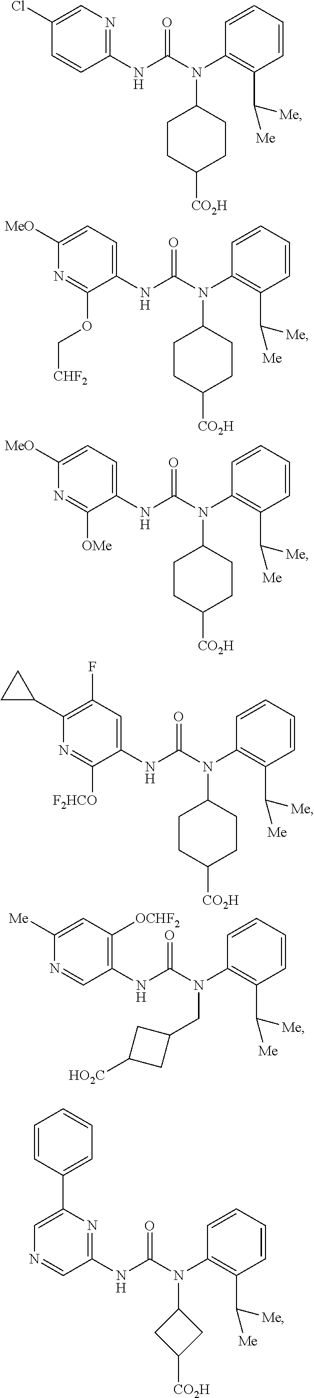

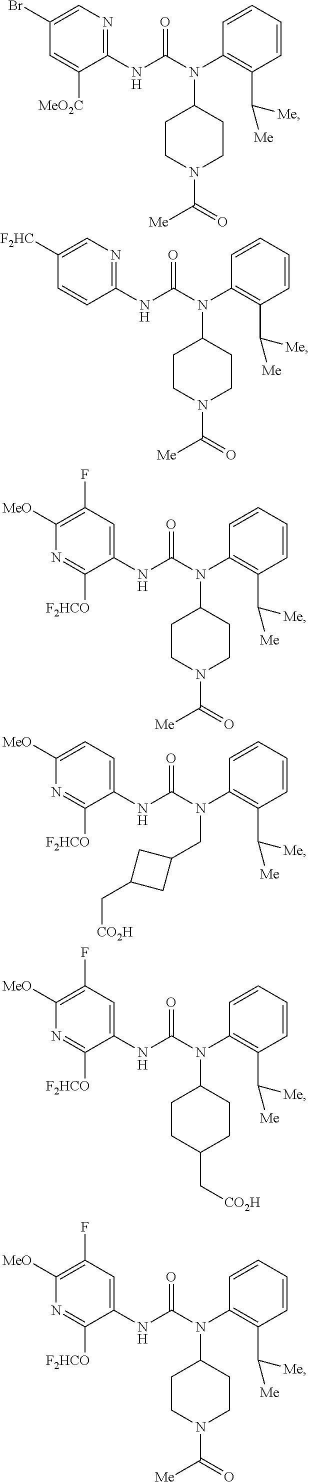

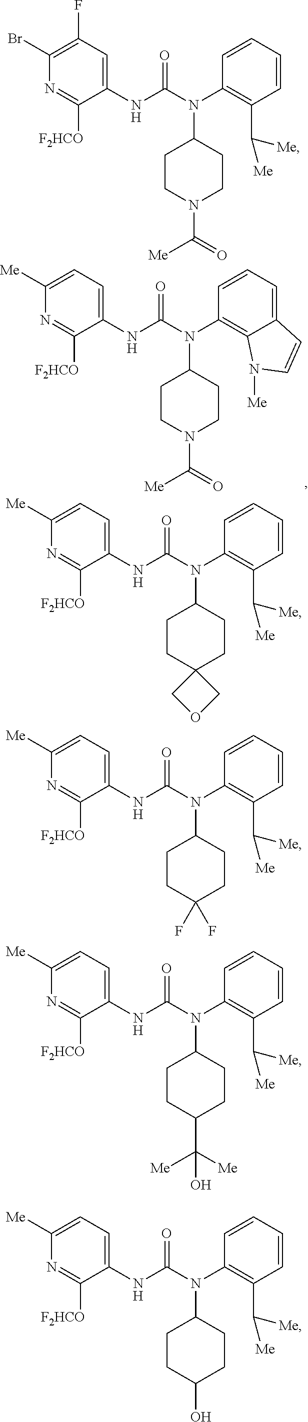

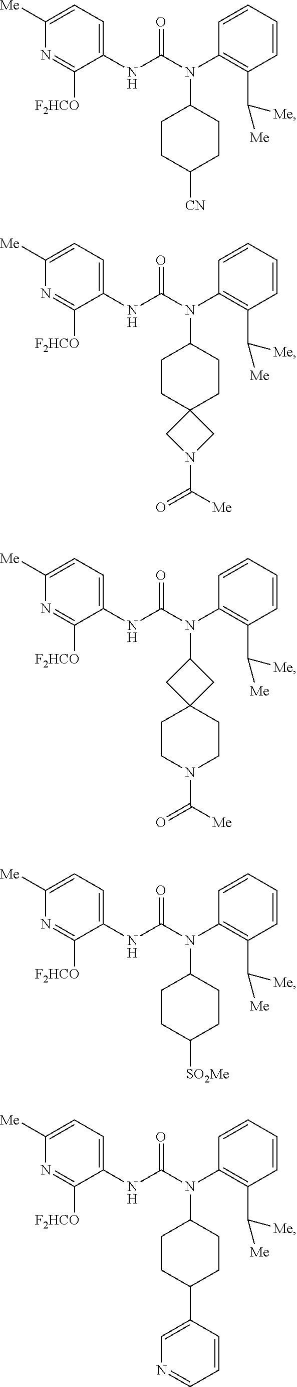 [SMILES: Cc1ccc(NC(=O)N(c2ccccc2C(C)C)C2CCC(C#N)CC2)c(OC(F)F)n1 ; CC(=O)N1CC2(CCC(N(C(=O)Nc3ccc(C)nc3OC(F)F)c3ccccc3C(C)C)CC2)C1 ; CC(=O)N1CCC2(CC1)CC(N(C(=O)Nc1ccc(C)nc1OC(F)F)c1ccccc1C(C)C)C2 ; Cc1ccc(NC(=O)N(c2ccccc2C(C)C)C2CCC(S(C)(=O)=O)CC2)c(OC(F)F)n1 ; Cc1ccc(NC(=O)N(c2ccccc2C(C)C)C2CCC(c3cccnc3)CC2)c(OC(F)F)n1]

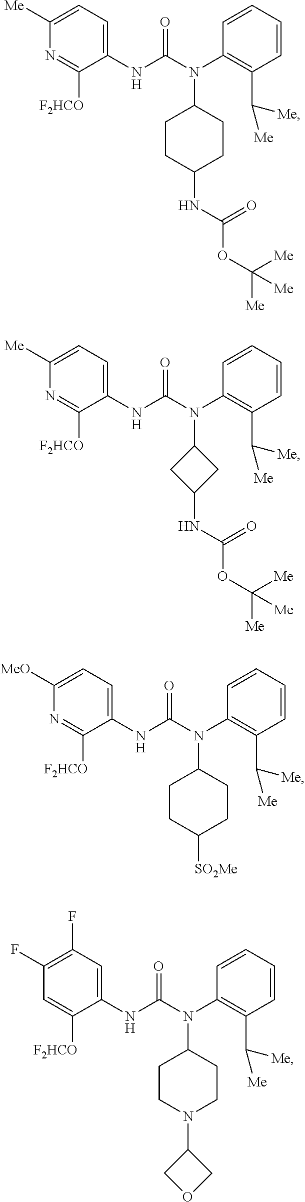

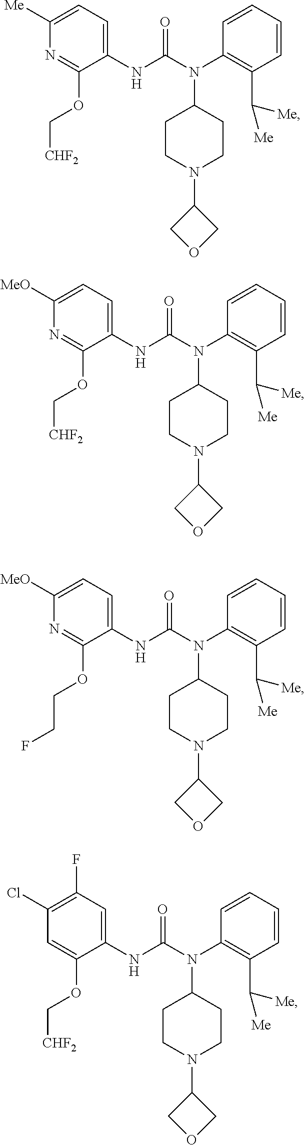 [SMILES: Cc1ccc(NC(=O)N(c2ccccc2C(C)C)C2CCN(C3COC3)CC2)c(OCC(F)F)n1 ; COc1ccc(NC(=O)N(c2ccccc2C(C)C)C2CCN(C3COC3)CC2)c(OCC(F)F)n1 ; COc1ccc(NC(=O)N(c2ccccc2C(C)C)C2CCN(C3COC3)CC2)c(OCCF)n1 ; CC(C)c1ccccc1N(C(=O)Nc1cc(F)c(Cl)cc1OCC(F)F)C1CCN(C2COC2)CC1]

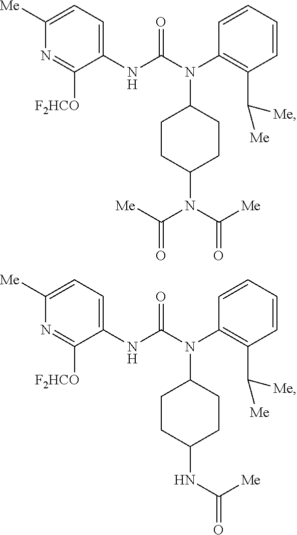

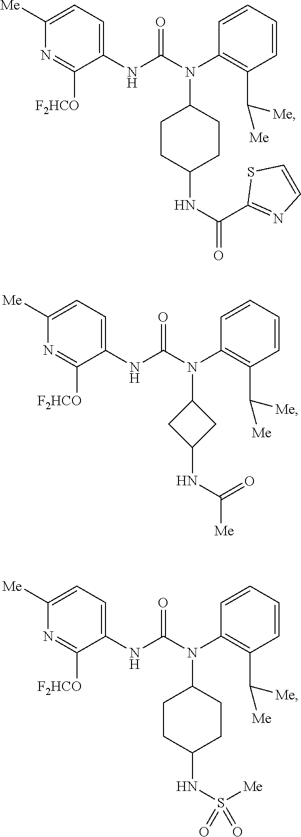 [SMILES: Cc1ccc(NC(=O)N(c2ccccc2C(C)C)C2CCC(NC(=O)c3nccs3)CC2)c(OC(F)F)n1 ; CC(=O)NC1CC(N(C(=O)Nc2ccc(C)nc2OC(F)F)c2ccccc2C(C)C)C1 ; Cc1ccc(NC(=O)N(c2ccccc2C(C)C)C2CCC(NS(C)(=O)=O)CC2)c(OC(F)F)n1]

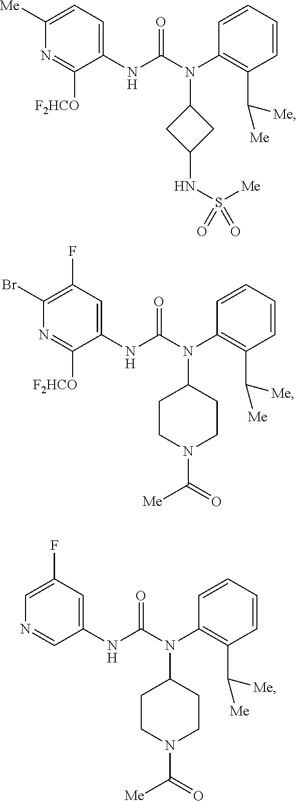 [SMILES: Cc1ccc(NC(=O)N(c2ccccc2C(C)C)C2CC(NS(C)(=O)=O)C2)c(OC(F)F)n1 ; CC(=O)N1CCC(N(C(=O)Nc2cc(F)c(Br)nc2OC(F)F)c2ccccc2C(C)C)CC1 ; CC(=O)N1CCC(N(C(=O)Nc2cncc(F)c2)c2ccccc2C(C)C)CC1]

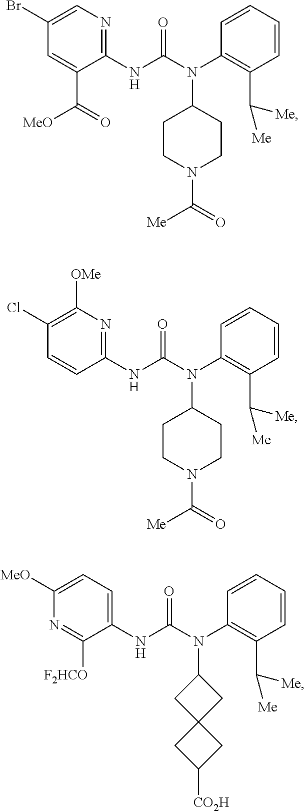 [SMILES: COC(=O)c1cc(Br)cnc1NC(=O)N(c1ccccc1C(C)C)C1CCN(C(C)=O)CC1 ; COc1nc(NC(=O)N(c2ccccc2C(C)C)C2CCN(C(C)=O)CC2)ccc1Cl ; COc1ccc(NC(=O)N(c2ccccc2C(C)C)C2CC3(CC(C(=O)O)C3)C2)c(OC(F)F)n1]

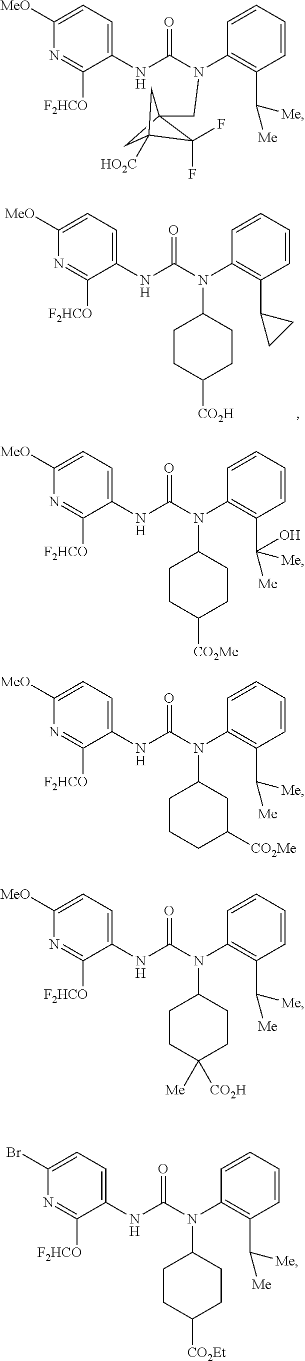

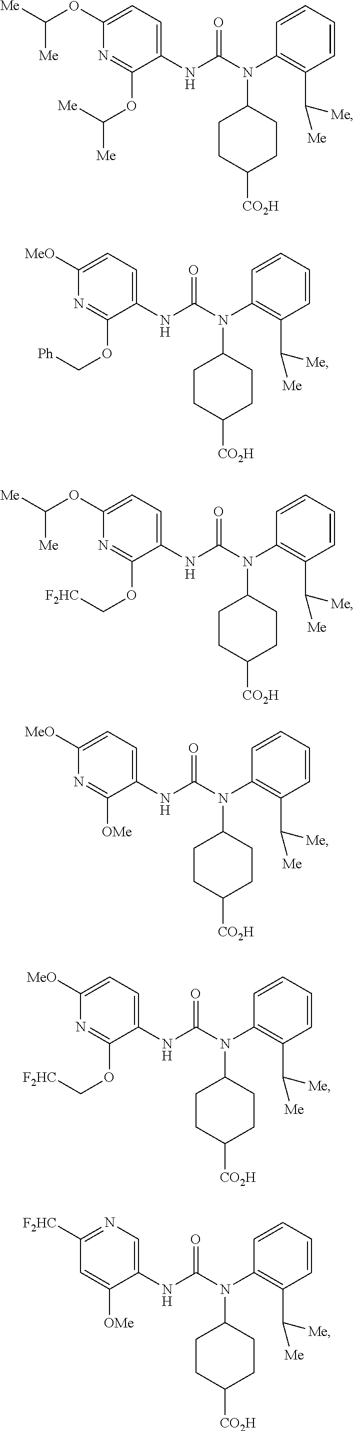 [SMILES: CC(C)Oc1ccc(NC(=O)N(c2ccccc2C(C)C)C2CCC(C(=O)O)CC2)c(OC(C)C)n1 ; COc1ccc(NC(=O)N(c2ccccc2C(C)C)C2CCC(C(=O)O)CC2)c(OCc2ccccc2)n1 ; CC(C)Oc1ccc(NC(=O)N(c2ccccc2C(C)C)C2CCC(C(=O)O)CC2)c(OCC(F)F)n1 ; COc1ccc(NC(=O)N(c2ccccc2C(C)C)C2CCC(C(=O)O)CC2)c(OC)n1 ; COc1ccc(NC(=O)N(c2ccccc2C(C)C)C2CCC(C(=O)O)CC2)c(OCC(F)F)n1 ; COc1cc(C(F)F)ncc1NC(=O)N(c1ccccc1C(C)C)C1CCC(C(=O)O)CC1]

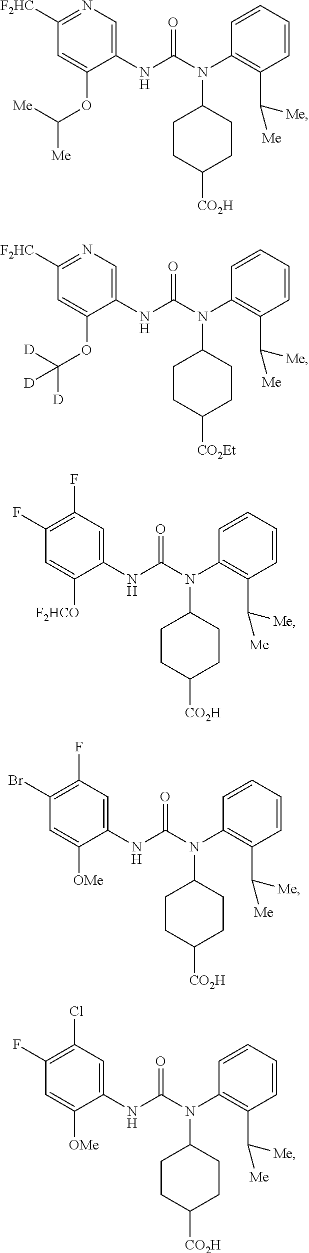 [SMILES: CC(C)Oc1cc(C(F)F)ncc1NC(=O)N(c1ccccc1C(C)C)C1CCC(C(=O)O)CC1 ; [2H]C([2H])([2H])Oc1cc(C(F)F)ncc1NC(=O)N(c1ccccc1C(C)C)C1CCC(C(=O)OCC)CC1 ; CC(C)c1ccccc1N(C(=O)Nc1cc(F)c(F)cc1OC(F)F)C1CCC(C(=O)O)CC1 ; COc1cc(Br)c(F)cc1NC(=O)N(c1ccccc1C(C)C)C1CCC(C(=O)O)CC1 ; COc1cc(F)c(Cl)cc1NC(=O)N(c1ccccc1C(C)C)C1CCC(C(=O)O)CC1]

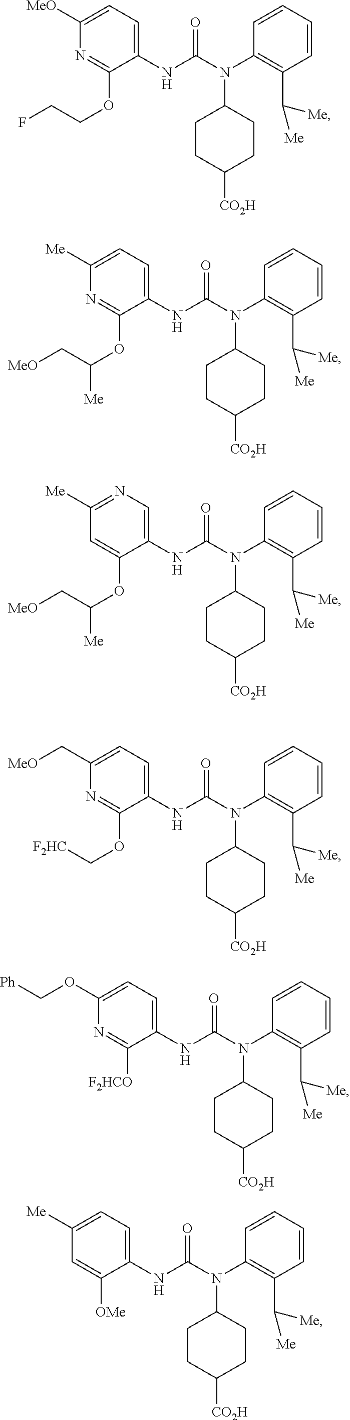

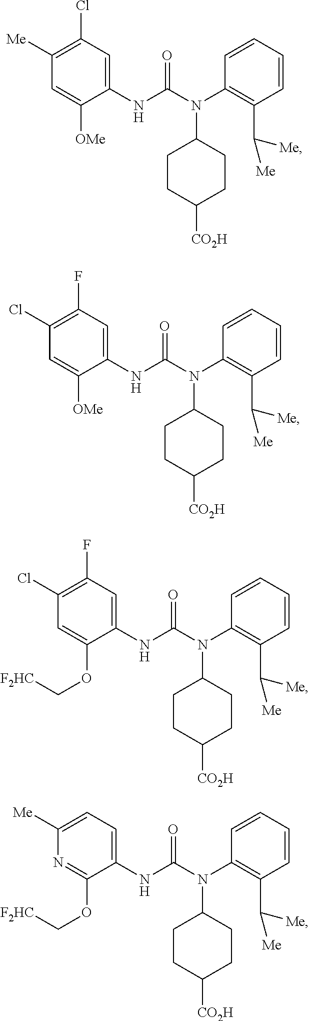 [SMILES: COc1cc(C)c(Cl)cc1NC(=O)N(c1ccccc1C(C)C)C1CCC(C(=O)O)CC1 ; COc1cc(Cl)c(F)cc1NC(=O)N(c1ccccc1C(C)C)C1CCC(C(=O)O)CC1 ; CC(C)c1ccccc1N(C(=O)Nc1cc(F)c(Cl)cc1OCC(F)F)C1CCC(C(=O)O)CC1 ; Cc1ccc(NC(=O)N(c2ccccc2C(C)C)C2CCC(C(=O)O)CC2)c(OCC(F)F)n1]

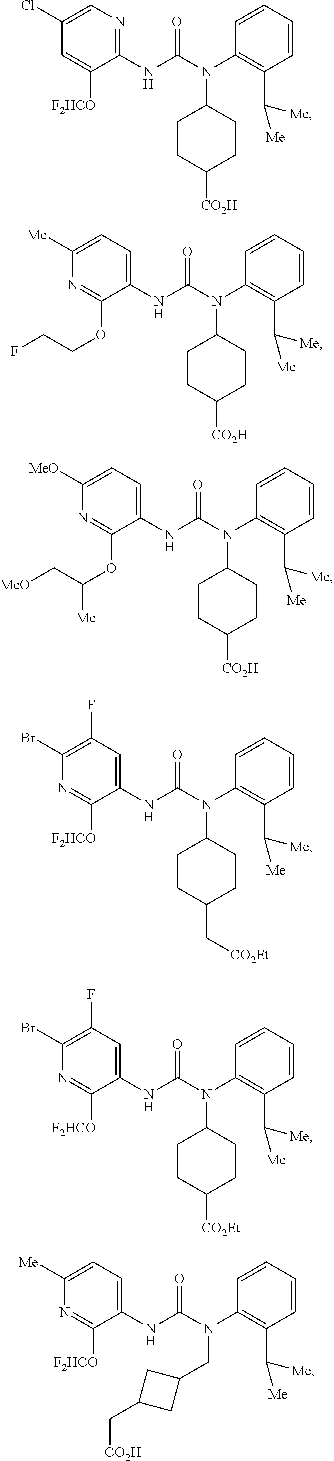 [SMILES: CC(C)c1ccccc1N(C(=O)Nc1ncc(Cl)cc1OC(F)F)C1CCC(C(=O)O)CC1 ; Cc1ccc(NC(=O)N(c2ccccc2C(C)C)C2CCC(C(=O)O)CC2)c(OCCF)n1 ; COCC(C)Oc1nc(OC)ccc1NC(=O)N(c1ccccc1C(C)C)C1CCC(C(=O)O)CC1 ; CCOC(=O)CC1CCC(N(C(=O)Nc2cc(F)c(Br)nc2OC(F)F)c2ccccc2C(C)C)CC1 ; CCOC(=O)C1CCC(N(C(=O)Nc2cc(F)c(Br)nc2OC(F)F)c2ccccc2C(C)C)CC1 ; Cc1ccc(NC(=O)N(CC2CC(CC(=O)O)C2)c2ccccc2C(C)C)c(OC(F)F)n1]

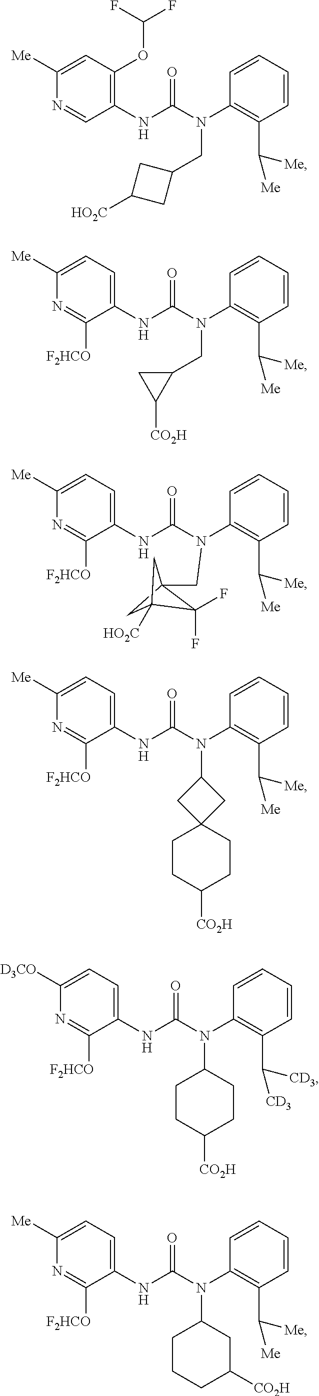

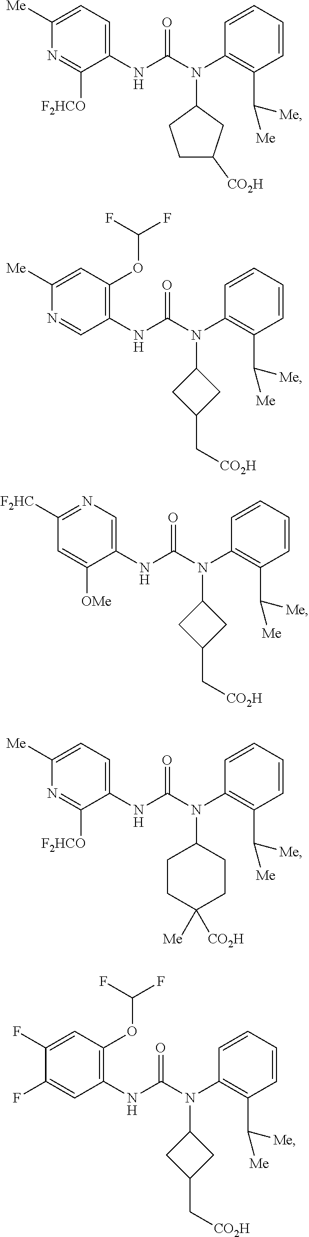

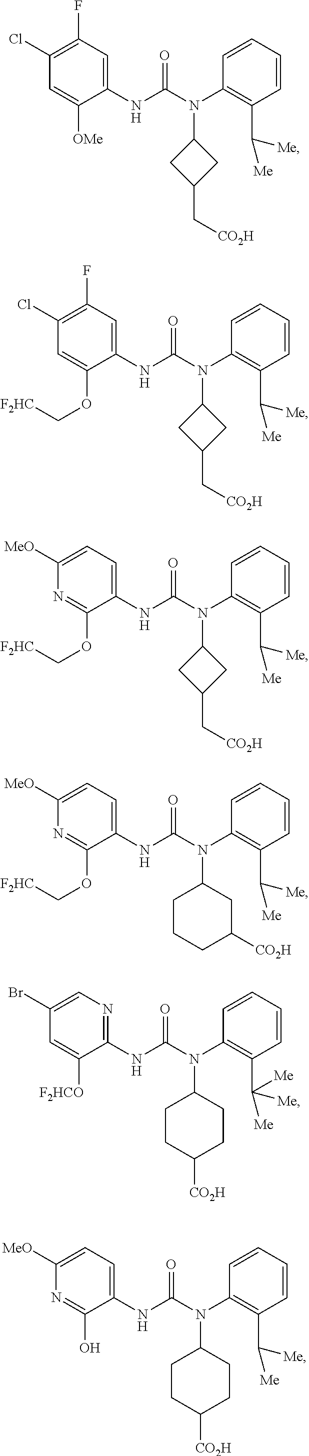

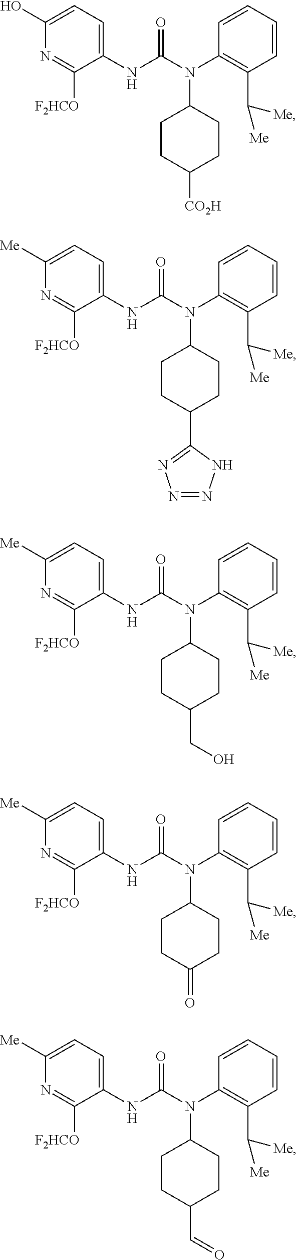 [SMILES: CC(C)c1ccccc1N(C(=O)Nc1ccc(O)nc1OC(F)F)C1CCC(C(=O)O)CC1 ; Cc1ccc(NC(=O)N(c2ccccc2C(C)C)C2CCC(c3nnn[nH]3)CC2)c(OC(F)F)n1 ; Cc1ccc(NC(=O)N(c2ccccc2C(C)C)C2CCC(CO)CC2)c(OC(F)F)n1 ; Cc1ccc(NC(=O)N(c2ccccc2C(C)C)C2CCC(=O)CC2)c(OC(F)F)n1 ; Cc1ccc(NC(=O)N(c2ccccc2C(C)C)C2CCC(C=O)CC2)c(OC(F)F)n1]

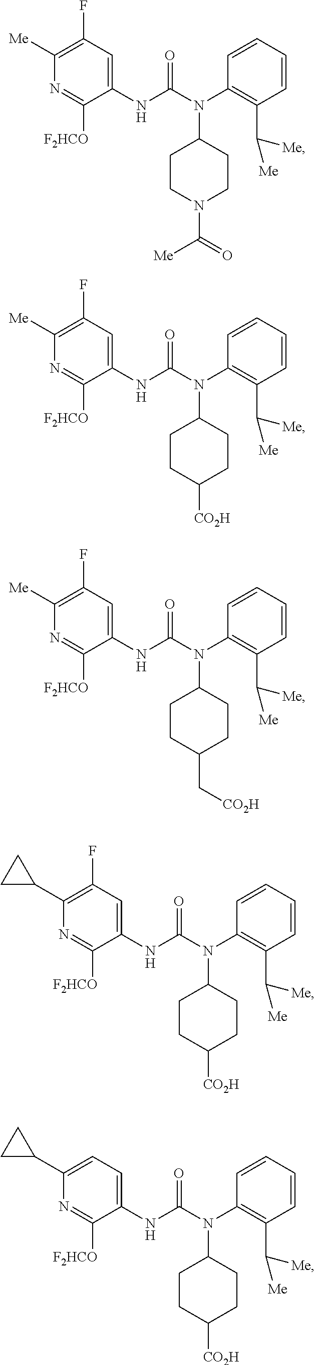 [SMILES: CC(=O)N1CCC(N(C(=O)Nc2cc(F)c(C)nc2OC(F)F)c2ccccc2C(C)C)CC1 ; Cc1nc(OC(F)F)c(NC(=O)N(c2ccccc2C(C)C)C2CCC(C(=O)O)CC2)cc1F ; Cc1nc(OC(F)F)c(NC(=O)N(c2ccccc2C(C)C)C2CCC(CC(=O)O)CC2)cc1F ; CC(C)c1ccccc1N(C(=O)Nc1cc(F)c(C2CC2)nc1OC(F)F)C1CCC(C(=O)O)CC1 ; CC(C)c1ccccc1N(C(=O)Nc1ccc(C2CC2)nc1OC(F)F)C1CCC(C(=O)O)CC1]

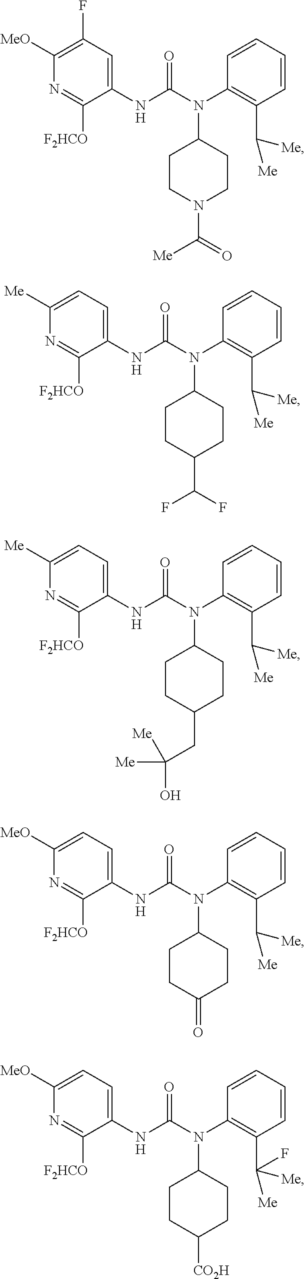

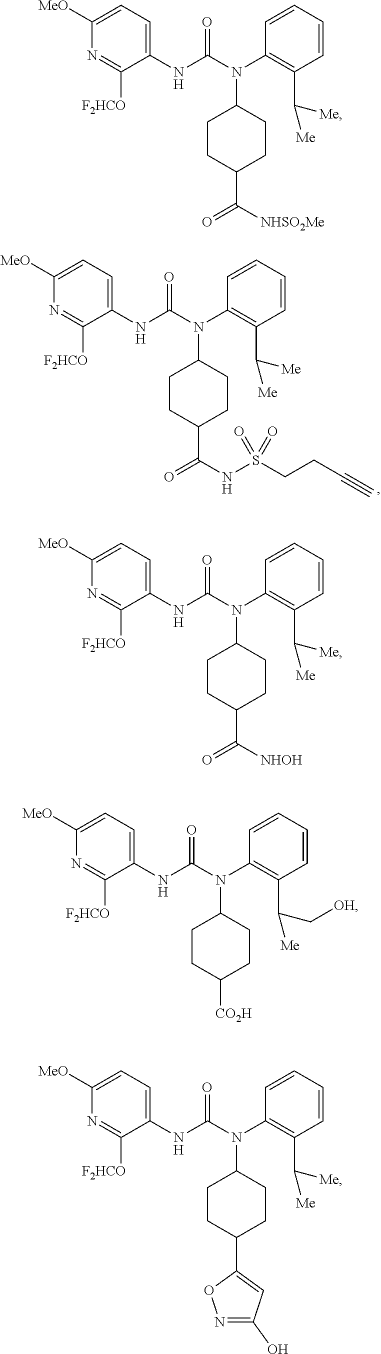

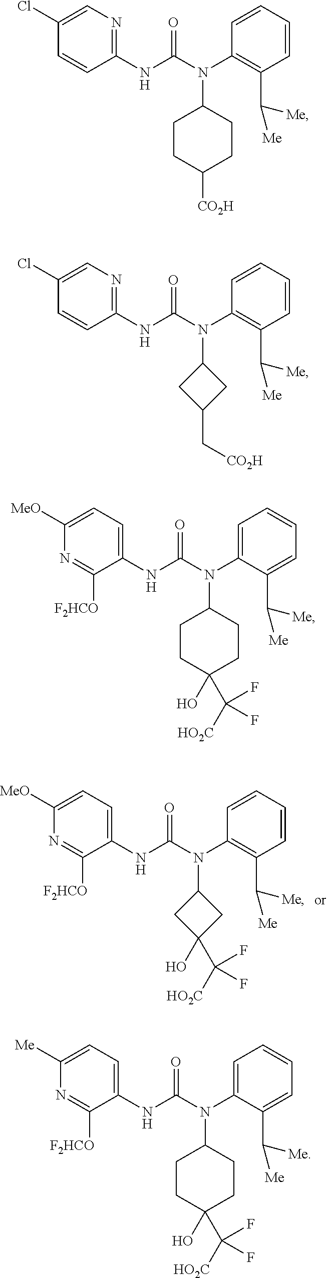 [SMILES: CC(C)c1ccccc1N(C(=O)Nc1ccc(Cl)cn1)C1CCC(C(=O)O)CC1 ; CC(C)c1ccccc1N(C(=O)Nc1ccc(Cl)cn1)C1CC(CC(=O)O)C1 ; COc1ccc(NC(=O)N(c2ccccc2C(C)C)C2CCC(O)(C(F)(F)C(=O)O)CC2)c(OC(F)F)n1 ; COc1ccc(NC(=O)N(c2ccccc2C(C)C)C2CC(O)(C(F)(F)C(=O)O)C2)c(OC(F)F)n1 ; "or" ; Cc1ccc(NC(=O)N(c2ccccc2C(C)C)C2CCC(O)(C(F)(F)C(=O)O)CC2)c(OC(F)F)n1]

Embodiment 41. A pharmaceutical composition comprising the compound of one of embodiments 1 to 40, or a pharmaceutically acceptable salt or solvate thereof, and a pharmaceutically acceptable excipient.

Embodiment 42. A method of treating a neurodegenerative disorder in a subject in need thereof, said method comprising administering to the subject in need thereof a therapeutically effective amount of a compound of one of embodiments 1 to 40, or a pharmaceutically acceptable salt or solvate thereof.

Embodiment 43. A method of treating an inflammatory disease in a subject in need thereof, said method comprising administering to the subject in need thereof a therapeutically effective amount of a compound of one of embodiments 1 to 40, or a pharmaceutically acceptable salt or solvate thereof.

Embodiment 44. The method of embodiment 43, wherein the inflammatory disease is encephalitis.

Embodiment 45. The method of embodiment 44, wherein the encephalitis is post-hemorrhagic encephalitis.

Embodiment 46. A method of treating a demyelinating disease in a subject in need thereof, said method comprising administering to the subject in need thereof a therapeutically effective amount of a compound of one of embodiments 1 to 40, or a pharmaceutically acceptable salt or solvate thereof.

Embodiment 47. The method of embodiment 46, wherein the demyelinating disease is a demyelinating disease of the central nervous system.

Embodiment 48. The method of embodiment 47, wherein the demyelinating disease is multiple sclerosis.

Embodiment 49. The method of embodiment 46, wherein the demyelinating disease is a demyelinating disease of the peripheral nervous system.

Embodiment 50. A method of treating a fibrotic disease in a subject in need thereof, said method comprising administering to the subject in need thereof a therapeutically effective amount of a compound of one of embodiments 1 to 40, or a pharmaceutically acceptable salt or solvate thereof.

Embodiment 51. The method of embodiment 50, wherein the fibrotic disease is pulmonary fibrosis, skin fibrosis, liver fibrosis, or ocular fibrosis.

Embodiment 52. The method of embodiment 50, wherein the fibrotic disease is idiopathic pulmonary fibrosis, scleroderma, nonalcoholic steatohepatitis, or ocular fibrosis.

Embodiment 53. A method of treating cancer in a subject in need thereof, said method comprising administering to the subject in need thereof a therapeutically effective amount of a compound of one of embodiments 1 to 40, or a pharmaceutically acceptable salt or solvate thereof.

Embodiment 54. The method of embodiment 53, wherein the cancer is brain cancer.

Embodiment 55. The method of embodiment 54, wherein the cancer is glioblastoma.

Embodiment 56. A method of modulating LPAR1 activity in a subject, said method comprising administering to the subject a compound of one of embodiments 1 to 40, or a pharmaceutically acceptable salt or solvate thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Multiple sclerosis (MS), a chronic progressive disorder, is an inflammatory autoimmune disease whereby the dysregulated immune system attacks the patient's own protective myelin sheath. If left unchecked, the signal transduction along denuded axons can become increasingly deteriorated, eventually leading to the permanent damage of the affected nerve fibers. Recent studies have shown that, when compared to patients with non-inflammatory, non-vascular neurological diseases, MS patients have increased levels of LPAs in the serum (Balood et al., Hum. Immunol., 2014, 75, 411-413). Furthermore, in MS patients, the LPA levels in serum and cerebral spinal fluid (CSF) are even further elevated during disease flares when compared to periods of remission (Jiang et al., Neurol Res., 2018, 40, 335-339). This is consistent with the increased autotaxin activity, an enzyme known to be important for the generation of LPA from lysophosphatidylcholine, observed in MS patients (Zahednasab et al., J. Neuroimmunol., 2014, 273, 120-123). Indeed, it has been established in cellular assays that LPA can induce a pro-inflammatory response through the activation of LPAR1 found in MS patient monocyte-derived macrophages by stimulating the production of pro-inflammatory cytokines such as interleukin-1β (IL-1β) and tumor necrosis factor alpha (TNFα), as well as suppressing the production of anti-inflammatory cytokines such as interleukin-2 (IL-2). Furthermore, in the mouse experimental autoimmune encephalomyelitis (EAE) model of MS, blockade of LPA signaling through genetic deletion of LPA1 was found to decrease the severity of the disease (Fransson et al., Mol. Neurobiol., 2021, 58, 470-482). While dampening the inflammatory immune attack on the myelin sheath can constitute an effective approach to the treatment of MS, promoting the remyelination of damaged, denuded axons would represent another attractive approach (Deshmukh et al., Nature, 2013, 502, 327-332). In this regard, neuronal remyelination can be achieved by driving the differentiation of oligodendrocyte precursor cells (OPCs), an endogenous stem cell, to myelin-producing oligodendrocytes (Najm et al., Nature, 2015, 52, 216-220). It has been demonstrated that LPAR1 is highly expressed in OPCs. Furthermore, a direct role of LPAR1 in OPC differentiation in mice has been established whereby the genetic knockout of LPAR1 led to an enrichment of oligodendrocytes and overexpression of myelin-protein positive (MBP) cells in the mouse cortex (Lorrain et al., Society for Neuroscience Conference Proceedings, 2017, “LPA receptors modulate oligodendrocyte differentiation and maturation”). Since LPA-LPAR1 signaling axis has been shown to play an important role in both demyelination (i.e., promotion of an inflammatory environment) and remyelination (i.e., prevention of OPC differentiation into oligodendrocyte) of axons, the identification of a potent and selective LPAR1 antagonists would thus be of significant relevance in the treatment of MS and other inflammatory demyelination disorders.

### 1. Experimental Procedures and Characterization Data

The compounds used in the reactions described herein are made according to known organic synthesis techniques, starting from commercially available chemicals and/or from compounds described in the chemical literature. “Commercially available chemicals” are obtained from standard commercial sources including Acros Organics (Geel, Belgium), Aldrich Chemical (Milwaukee, WI, including Sigma Chemical and Fluka), Apin Chemicals Ltd. (Milton Park, UK), Ark Pharm, Inc. (Libertyville, IL), Avocado Research (Lancashire, U.K.), BDH Inc. (Toronto, Canada), Bionet (Cornwall, U.K.), Chemservice Inc. (West Chester, PA), Combi-blocks (San Diego, CA), Crescent Chemical Co. (Hauppauge, NY), eMolecules (San Diego, CA), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Matrix Scientific, (Columbia, SC), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Ryan Scientific, Inc. (Mount Pleasant, SC), Spectrum Chemicals (Gardena, CA), Sundia Meditech, (Shanghai, China), TCI America (Portland, OR), Trans World Chemicals, Inc. (Rockville, MD), and WuXi (Shanghai, China).

Suitable reference books and treatises that detail the synthesis of reactants useful in the preparation of compounds described herein, or provide references to articles that describe the preparation, include for example, “Synthetic Organic Chemistry,” New York: John Wiley & Sons, Inc., 1982; Sandler S. R. et al., “Organic Functional Group Preparations,” 2^{nd}ed., New York: Academic Press, 1983; House, H. O., “Modern Synthetic Reactions,” 2^{nd} ed., Menlo Park: W. A. Benjamin, Inc., 1972; Gilchrist, T. L., “Heterocyclic Chemistry,” 2^{nd} ed., New York: Wiley, 1992; March, J., “Advanced Organic Chemistry: Reactions, Mechanisms and Structure,” 4^{th} ed., New York: Wiley, 1992. Additional suitable reference books and treatises that detail the synthesis of reactants useful in the preparation of compounds described herein, or provide references to articles that describe the preparation, include for example, Fuhrhop, J., Penzlin, G., “Organic Synthesis: Concepts, Methods, Starting Materials,” 2^{nd} ed., New York: Wiley, 1994; Hoffman, R. V., “Organic Chemistry, An Intermediate Text,” Oxford: Oxford University Press, 1996; Larock, R. C., “Comprehensive Organic Transformations: A Guide to Functional Group Preparations,” 2^{nd} ed., New York: Wiley, 1999; Otera, J., “Modern Carbonyl Chemistry,” New York: Wiley, 2000; Solomons, T. W. G., “Organic Chemistry,” 7^{th} ed., New York: Wiley, 2000; Stowell, J. C., “Intermediate Organic Chemistry,” 2^{nd} ed., New York: Wiley, 1993; “Industrial Organic Chemicals: Starting Materials and Intermediates: An Ullmann's Encyclopedia,” New York: Wiley, in 8 volumes; “Organic Reactions,” New York: Wiley, in over 55 volumes; and “Chemistry of Functional Groups,” New York: Wiley, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is Stahl, P. H., Wermuth, C. G., “Handbook of Pharmaceutical Salts,” Zurich: Verlag Helvetica Chimica Acta, 2002.

### List of Abbreviations

As used above, and throughout the description of the invention, the following abbreviations, unless otherwise indicated, shall be understood to have the following meanings:
ACN or MeCN acetonitrile
aq aqueous
BAST bis(2-methoxyethyl)aminosulfur trifluoride
Bu butyl
BOC or Boc tert-butyl carbamate
BrettPhos Pd G3 [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate
BSA bovine serum albumin
CDI 1,1′-carbonyldiimidazole
CHO Chinese hamster ovary
Cy cyclohexyl
dba dibenzylideneacetone
DAST diethylaminosulfur trifluoride
DCC N,N′-dicyclohexylcarbodiimide
DCE dichloroethane (ClCH₂CH₂Cl)
DCM dichloromethane (CH₂Cl₂)
DIPEA or DIEA N,N-diisopropylethylamine
DMAP 4-(N,N-dimethylamino)pyridine
DME 1,2-dimethoxyethane
DMEM Dulbecco's modified eagle medium
DMF N,N-dimethylformamide
DMA N,N-dimethylacetamide
DMSO dimethylsulfoxide
EDC N-(3-dimethylaminopropyl)-N′-ethylcarbodiimide
equiv equivalent(s)
Et ethyl
EtOH ethanol
EtOAc ethyl acetate
FBS fetal bovine serum
h hour(s)
HATU 1-[bis(dimethylamino)methylene]-1H-1,2,3-triazolo[4,5-b]pyridinium 3-oxide hexafluorophosphate
HEPES 4-(2-hydroxyethyl)-1-piperazineethanesulfonic acid
Hex hexanes
HPLC high performance liquid chromatography
LCMS or LC-MS liquid chromatography-mass spectrometry
LG leaving group
M molar
mCPBA meta-chloroperoxybenzoic acid
Me methyl
MeOH methanol
min minute(s)
MS mass spectroscopy
NMI N-methylimidazole
NMP N-methyl-2-pyrrolidone
NMR nuclear magnetic resonance
Pd/C palladium on carbon
PG protecting group
RT room temperature
T3P propylphosphonic anhydride
TBAF tetrabutylammonium fluoride
TCFH chloro-N,N,N′,N′-tetramethylformamidinium hexafluorophosphate
TEA triethylamine
TFA trifluoroacetic acid
TFAA trifluoroacetic anhydride
THF tetrahydrofuran
v/v volume per volume
w/w weight per weight
XantPhos 4,5-bis(diphenylphosphino)-9,9-dimethylxanthene
XPhos 2-dicyclohexylphosphino-2′4′6′-triisopropylbiphenyl
XtalFluor-E (diethylamino)difluorosulfonium tetrafluoroborate

### General Synthetic Schemes

Compounds of Formula (I) of the present disclosure may be prepared, for example, from the union of a primary (hetero)aryl amine (1), or its corresponding (hetero)aryl ammonium salt, with a secondary (hetero)aryl amine (2), or its corresponding (hetero)aryl ammonium salt, in the presence of an appropriate “C═O” source such as CDI, phosgene, triphosgene, or the like, and an appropriate base such as pyridine, TEA, DIEA, sodium hydride, or the like (Scheme 1). Depending on the reactivity of the two amine coupling partners, it may be advantageous to first react either amine (1) or amine (2) with the aforementioned “C═O” source and base, to deliver the requisite carbamoyl chloride, carbamoyl imidazole, or isocyanate intermediate, prior to the addition of the other amine coupling partner. In some cases, the formation of the final urea bond can be promoted with the addition of activators such as DMAP, freshly activated molecular sieves, heat, or the like.

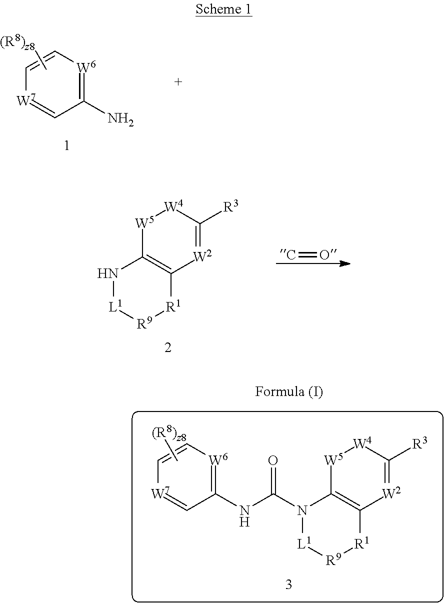

The product urea (3), which itself may be a compound of Formula (I), can be further functionalized using synthetic methodologies known to those skilled in the art to deliver another compound of Formula (I). Examples of such transformations include, but are not limited to:
(a) hydrolysis of an ester present in (3), with a suitable reagent such as lithium hydroxide, sodium hydroxide, or potassium hydroxide, or the like;
(b) hydrolysis of a nitrile present in (3), with a suitable reagent such as wet sulfuric acid, wet hydrochloric acid, lithium hydroxide, sodium hydroxide, or the like;
(c) cycloaddition of a nitrile present in (3), with a suitable azide such as trimethylsilyl azide, sodium azide, tetrabutylammonium azide, or the like, in the presence of a suitable promoter such as dibutyltin(IV) oxide, protic acid, heat, or the like;
(d) capping of an unmasked amine or alcohol present in (3), revealed following the removal of the masking group using conditions known to those skilled in the art, with a suitable electrophile such as acyl chloride, chloroformate, carbamoyl chloride, sulfonyl chloride, alkyl halide, isocyanate, or the like, in the presence of a suitable base such as TEA, pyridine, sodium hydride, cesium carbonate, or the like;
(e) coupling of an unmasked amine present in (3), revealed following the removal of the masking group using conditions known to those skilled in the art, with an appropriately functionalized carboxylic acid, in the presence of a suitable coupling reagent such as HATU, CDI, T3P, or the like, and a suitable base such as TEA, DIEA, NMI, or the like;
(f) coupling of an unmasked carboxylic acid present in (3), revealed following the removal of the masking group using conditions known to those skilled in the art, with an appropriately functionalized amine, in the presence of a suitable coupling reagent such as HATU, CDI, T3P, or the like, and a suitable base such as TEA, DIEA, NMI, or the like;
(g) hydrogenation of an alkene or an alkyne present in (3) and/or hydrogenolysis of an (hetero)aryl halide present in (3), in the presence of a suitable catalyst such as Pd/C, Pd(OH)₂, or the like, and a suitable reducing agent such as hydrogen gas, deuterium gas, or the like;
(h) reaction of an unmasked ketone or aldehyde present in (3), revealed following the removal of the masking group using conditions known to those skilled in the art, with a suitable organometallic reagent such as organolithiums, Grignard reagents, organozincates, organosilicon reagents, or the like;
(i) deoxyfluorination of an unmasked ketone, aldehyde, or alcohol present in (3), revealed following the removal of the masking group using conditions known to those skilled in the art, with a suitable reagent such as DAST, BAST, XtalFluor-E, or the like;
(j) reduction of an unmasked carboxylic acid, ketone, or aldehyde present in (3), revealed following the removal of the masking group using conditions known to those skilled in the art, with a suitable a reducing agent such as borane, lithium aluminum hydride, sodium borohydride, or the like;
(k) reductive amination of an unmasked ketone or aldehyde present in (3), revealed following the removal of the masking group using conditions known to those skilled in the art, with an appropriately functionalized amine, in the presence of a suitable a reducing agent such as sodium cyanoborohydride, sodium triacetoxyborohydride, or the like, and a suitable additive such as acetic acid, titanium(IV) isopropoxide, or the like;
(l) halogenation of (hetero)arene present in (3) with a suitable halogen source such as N-chlorosuccinimide, N-bromosuccinimide, iodine, or the like, in the presence of a suitable activator such as acetic acid, heat, or the like;
(m) coupling of an (hetero)aryl halide present in (3), with a suitable organotin reagent, organoboron reagent, organosilicon reagent, organozinc reagent, or the like, in the presence of a suitable promoter such as a palladium catalyst, an iron catalyst, a nickel catalyst, or the like, and a suitable base such as triethylamine, cesium carbonate, potassium phosphate, sodium bicarbonate, tetrabutylammonium fluoride, or the like;
(n) oxidation of a heteroarene present in (3), with a suitable oxidant such as oxone, mCPBA, or the like; and
(o) separation of a mixture of stereoisomers into its stereochemically-enriched constituents utilizing an appropriate chiral column such as ChiralPAK IF, CHIRAL ART Amylose SA, CHIRAL ART Cellulose SB, or the like.

For certain embodiments, a person skilled in the art can access the secondary (hetero)aryl amine (2) used for the coupling depicted in Scheme 1 by a Buchwald-Hartwig coupling of an appropriately functionalized amine (4) with an appropriately functionalized (hetero)aryl halide (5), in the presence of a promoter such as a palladium catalyst, a copper catalyst, or the like, and a base such as potassium phosphate, TEA, potassium bis(trimethylsilyl)amide, or the like (Scheme 2).

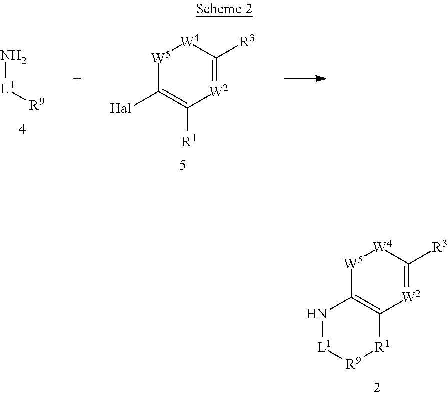

For certain embodiments, the secondary (hetero)aryl amine (2) used for the coupling depicted in Scheme 1 can instead be accessed from the reductive amination of an appropriately functionalized aldehyde or ketone (6) with an appropriately functionalized (hetero)aryl amine (7), in the presence of a suitable a reducing agent such as sodium cyanoborohydride, sodium triacetoxyborohydride, or the like, and a suitable additive such as acetic acid, titanium(IV) isopropoxide, or the like (Scheme 3).

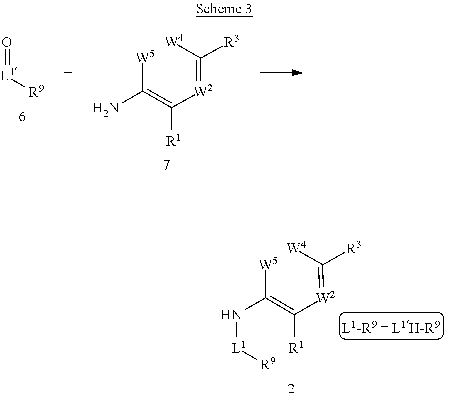

For certain embodiments, due to, for example, greater accessibility of the requisite starting materials or greater facility with which the requisite bond formation proceeds, it may be advantageous to delay the introduction of the R¹ group. In these instances, the secondary halo(hetero)aryl amine (9), itself synthesized from halo(hetero)arene (8) using the strategies disclosed previously, can be converted to the secondary (hetero)aryl amine (2) by its metal-catalyzed cross coupling with an appropriately functionalized organometallic reagent (10), using conditions known to those skilled in the art (Scheme 4). In certain cases, the conversion of (9) to (2) can require a two-step sequence involving an initial metal-catalyzed cross coupling with (10), followed by a hydrogenation of the intermediate alkene or alkyne.

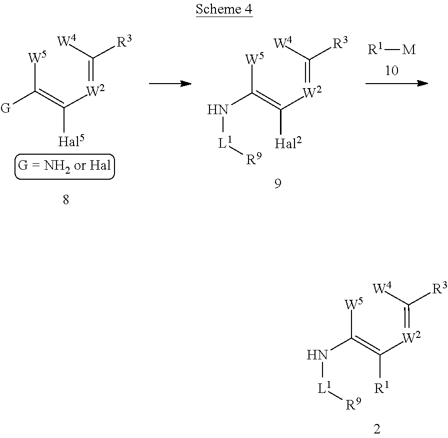

For certain embodiments where the primary (hetero)aryl amine (1) used for the coupling depicted in Scheme 1 is not commercially available, it may be prepared from the more readily available (hetero)aryl halide (11) by its Buchwald-Hartwig coupling with an appropriately “NH₂” source such as benzophenone imine, tert-butyl carbamate, lithium bis(trimethylsilyl)amide, ammonia, or the like, followed by, when appropriate, an unmasking step (Scheme 5).

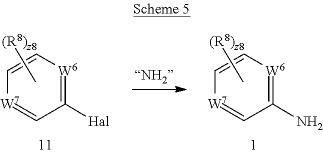

Alternatively, in instances where the preexisting electronic and/or steric determinants in (hetero)arene (12) allow for a regioselective nitration event, its conversion to primary (hetero)aryl amine (1) can be readily completed following the reduction of intermediate nitro (hetero)arene (13) using conditions known to those skilled in the art (Scheme 6).

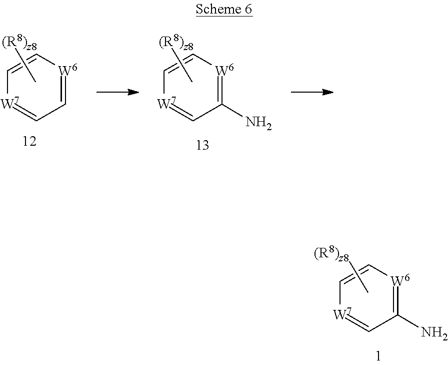

The general synthetic schemes above have been described in an illustrative manner and is intended to be in the nature of description rather than of limitation. It will also be appreciated that many of the reagents provided in the following examples may be substituted with other suitable reagents (see, e.g., is Fieser, L., et al., “Encyclopedia of Reagents for Organic Synthesis,” 2^{nd} ed., New York: Wiley, 2009). In addition, it will be appreciated that conditions such as choice of solvent, temperature of reaction, volumes and reaction time may vary while still producing the desired compounds. Such changes and modifications, including without limitation, those relating to the chemical structures, substituents, derivatives, intermediates and/or syntheses provided herein, may be made without departing from the spirit and scope thereof.

### EXAMPLES

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography was performed on silica gel unless otherwise noted.

### Intermediate amine 1: Preparation of tert-butyl 3-((2-isopropylphenyl)amino)azetidine-i-carboxylate

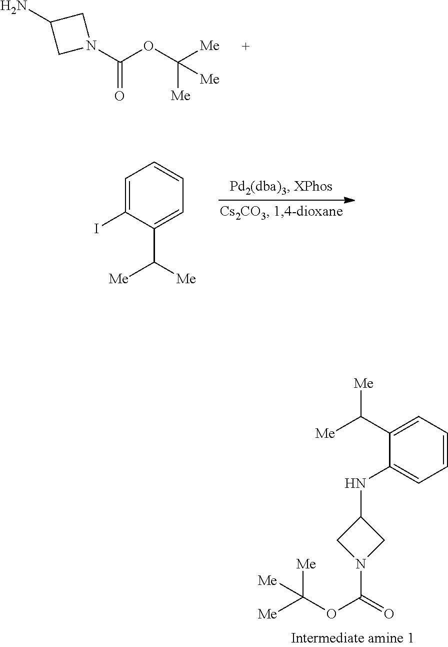 [SMILES: CC(C)(C)OC(=O)N1CC(N)C1 ; CC(C)c1ccccc1I ; CC(C)c1ccccc1NC1CN(C(=O)OC(C)(C)C)C1]

In a thick-walled glass reaction vessel equipped with a magnetic stirrer and a Teflon screwcap was combined tert-butyl 3-aminoazetidine-1-carboxylate (1 equiv, Combi-Blocks), 1-iodo-2-isopropylbenzene (1 equiv, Combi-Blocks), tris(dibenzylidineacetone)dipalladium(0) (0.04 equiv, Sigma-Aldrich), 2-dicyclohexylphosphino-2′4′6′-triisopropylbiphenyl (0.12 equiv, Combi-Blocks), and cesium carbonate (8 equiv, Sigma-Aldrich) in 1,4-dioxane (0.2 M). The resulting purple suspension was then deoxygenated via subsurface purging with nitrogen for 10 min before the reaction vessel was tightly sealed and heated at 100° C. for 24 h. The resulting dark brown suspension was cooled to RT, diluted with EtOAc, and washed sequentially with water and brine. The organic extract thus obtained was then dried over MgSO₄, filtered, and the filtrate concentrated in vacuo. Purification of the crude product thus obtained by way of column chromatography (SiO₂, gradient elution: Hex→7:3 (v/v) Hex: EtOAc) afforded the title compound as a yellow oil (44% yield).

The following amines were prepared in an analogous fashion to Intermediate amine 1, but substituting tert-butyl 3-aminoazetidine-1-carboxylate with the requisite, commercially available amine.

| Starting Material | Product |
|---|---|
| 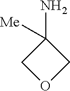 Combi-Blocks | 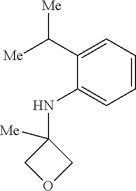 [SMILES: CC(C)c1ccccc1NC1(C)COC1] |
| | Intermediate amine 2 |
| 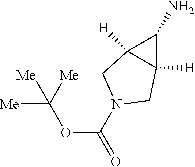 [SMILES: CC(C)(C)OC(=O)N1C[C@@H]2[C@@H](N)[C@@H]2C1] Combi-Blocks | 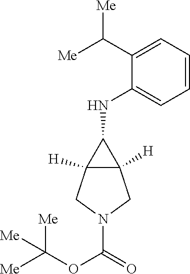 [SMILES: CC(C)c1ccccc1N[C@H]1[C@@H]2CN(C(=O)OC(C)(C)C)C[C@@H]21] |
| | Intermediate amine 3 |

### Intermediate amine 4: Preparation of tert-butyl 3-((2-isopropylphenyl)amino)pyrrolidine-1-carboxylate

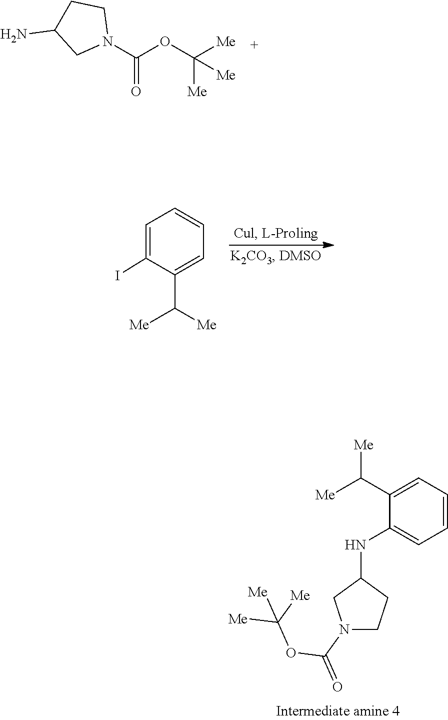 [SMILES: CC(C)(C)OC(=O)N1CCC(N)C1 ; CC(C)c1ccccc1I ; CC(C)c1ccccc1NC1CCN(C(=O)OC(C)(C)C)C1]

In a thick-walled glass reaction vessel equipped with a magnetic stirrer and a Teflon screwcap was combined tert-butyl 3-aminopyrrolidine-1-carboxylate (1 equiv, Combi-Blocks), 1-iodo-2-isopropylbenzene (1.2 equiv, Combi-Blocks), copper(I) iodide (0.15 equiv, Sigma-Aldrich), L-proline (0.3 equiv, Combi-Blocks), and potassium carbonate (3 equiv, Sigma-Aldrich) in DMSO (0.25 M). The resulting grey suspension was then deoxygenated via subsurface purging with nitrogen for 10 min before the reaction vessel was tightly sealed and heated at 100° C. for 48 h. The resulting suspension was cooled to RT, diluted with EtOAc, and washed sequentially with water and brine. The organic extract thus obtained was then dried over MgSO₄, filtered, and the filtrate concentrated in vacuo. Purification of the crude product thus obtained by way of column chromatography (SiO₂, gradient elution: Hex→7:3 (v/v) Hex: EtOAc) afforded the title compound as a yellow oil (33% yield).

### Intermediate amine 5: Preparation of tert-butyl 4-((2-isopropylphenyl)amino)piperidine-1-carboxylate

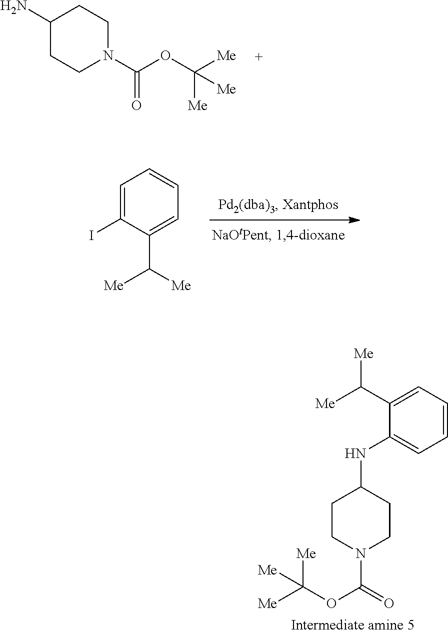 [SMILES: CC(C)(C)OC(=O)N1CCC(N)CC1 ; CC(C)c1ccccc1I ; CC(C)c1ccccc1NC1CCN(C(=O)OC(C)(C)C)CC1]

In a thick-walled glass reaction vessel equipped with a magnetic stirrer and a Teflon screwcap was combined tert-butyl 4-aminopiperidine-1-carboxylate hydrochloride (1.1 equiv, Combi-Blocks), 1-iodo-2-isopropylbenzene (1 equiv, Combi-Blocks), 4,5-bis(diphenylphosphino)-9,9-dimethylxanthene (0.1 equiv, Combi-Blocks), and sodium tert-pentoxide (4 equiv, Sigma-Aldrich) in 1,4-dioxane (0.15 M). The resulting yellow solution was deoxygenated via subsurface purging with nitrogen for 10 min and tris(dibenzylidineacetone)dipalladium(0) (0.05 equiv, Sigma-Aldrich) was then added. The reaction vessel was tightly sealed and heated at 100° C. for 48 h. The resulting dark brown suspension was cooled to RT, diluted with EtOAc, and washed sequentially with water and brine. The organic extract thus obtained was then dried over MgSO₄, filtered, and the filtrate concentrated in vacuo. Purification of the crude product thus obtained by way of column chromatography (SiO₂, gradient elution: Hex→4:1 (v/v) Hex: EtOAc) afforded the title compound as a yellow oil (84% yield).

The following amines were prepared in an analogous fashion to Intermediate amine 5, but substituting tert-butyl 4-aminopiperidine-1-carboxylate hydrochloride with the requisite, commercially available amine. For the synthesis of Intermediate amine 27, Intermediate amine 28, Intermediate amine 31, Intermediate amine 32, Intermediate amine 33, Intermediate amine 34, Intermediate amine 35, Intermediate amine 36, Intermediate amine 37, Intermediate amine 38, Intermediate amine 39, Intermediate amine 40, Intermediate amine 41, Intermediate amine 42, Intermediate amine 43, Intermediate amine 44, Intermediate amine 45, Intermediate amine 46, Intermediate amine 47, Intermediate amine 48, Intermediate amine 49, and Intermediate amine 50, sodium tert-pentoxide and 1,4-dioxane were also replaced with potassium tert-butoxide (Sigma-Aldrich) and toluene, respectively. For the synthesis of Intermediate amine 51, Intermediate amine 52, Intermediate amine 121, and Intermediate amine 122, sodium tert-pentoxide was also replaced with cesium carbonate (Sigma-Aldrich).

| Starting Material | Product |
|---|---|
| 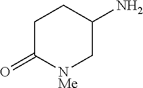 Combi-Blocks | 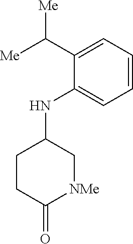 [SMILES: CC(C)c1ccccc1NC1CCC(=O)N(C)C1] |
| | Intermediate amine |
| | 6 |
| 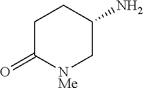 BioFine International | 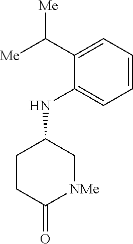 [SMILES: CC(C)c1ccccc1N[C@H]1CCC(=O)N(C)C1] |
| | Intermediate amine |
| | 7 |
| 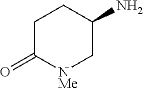 BioFine International | 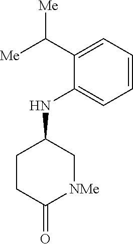 [SMILES: CC(C)c1ccccc1N[C@@H]1CCC(=O)N(C)C1] |
| | Intermediate amine |
| | 8 |
| 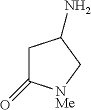 Combi-Blocks | 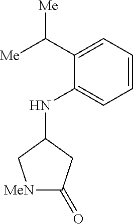 [SMILES: CC(C)c1ccccc1NC1CC(=O)N(C)C1] |
| | Intermediate amine |
| | 9 |
| 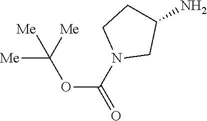 [SMILES: CC(C)(C)OC(=O)N1CC[C@H](N)C1] Combi-Blocks | 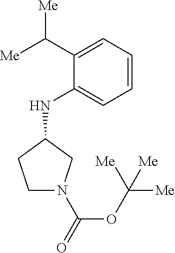 [SMILES: CC(C)c1ccccc1N[C@H]1CCN(C(=O)OC(C)(C)C)C1] |
| | Intermediate amine |
| | 10 |
| 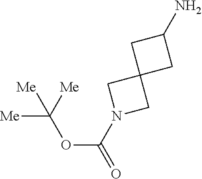 [SMILES: CC(C)(C)OC(=O)N1CC2(CC(N)C2)C1] Combi-Blocks | 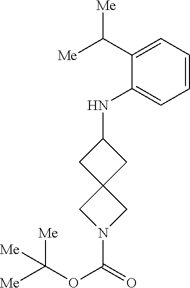 [SMILES: CC(C)c1ccccc1NC1CC2(C1)CN(C(=O)OC(C)(C)C)C2] |
| | Intermediate amine |
| | 11 |
| 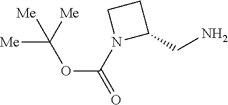 [SMILES: CC(C)(C)OC(=O)N1CC[C@@H]1CN] Combi-Blocks | 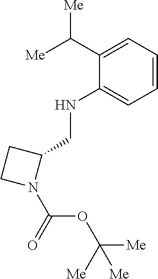 [SMILES: CC(C)c1ccccc1NC[C@H]1CCN1C(=O)OC(C)(C)C] |
| | Intermediate amine |
| | 12 |
| 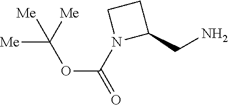 [SMILES: CC(C)(C)OC(=O)N1CC[C@H]1CN] Combi-Blocks | 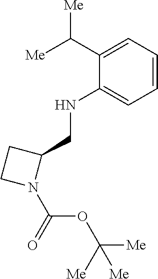 [SMILES: CC(C)c1ccccc1NC[C@@H]1CCN1C(=O)OC(C)(C)C] |
| | Intermediate amine |
| | 13 |
| 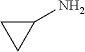 Combi-Blocks | 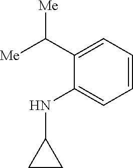 [SMILES: CC(C)c1ccccc1NC1CC1] |
| | Intermediate amine |
| | 14 |
| 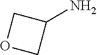 Combi-Blocks | 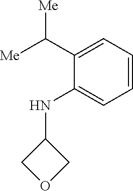 [SMILES: CC(C)c1ccccc1NC1COC1] |
| | Intermediate amine |
| | 15 |
| 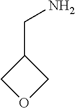 Combi-Blocks | 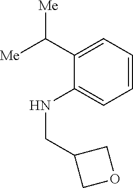 [SMILES: CC(C)c1ccccc1NCC1COC1] |
| | Intermediate amine |
| | 16 |
| 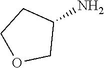 Combi-Blocks | 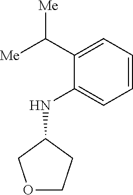 [SMILES: CC(C)c1ccccc1N[C@@H]1CCOC1] |
| | Intermediate amine |
| | 17 |
| 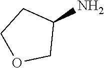 Combi-Blocks | 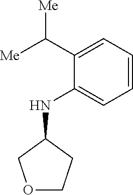 [SMILES: CC(C)c1ccccc1N[C@H]1CCOC1] |
| | Intermediate amine |
| | 18 |
| 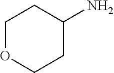 Combi-Blocks | 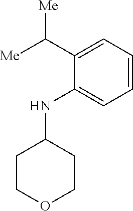 [SMILES: CC(C)c1ccccc1NC1CCOCC1] |
| | Intermediate amine |
| | 19 |
| 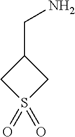 Enamine | 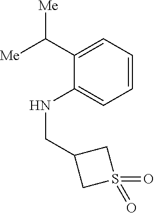 [SMILES: CC(C)c1ccccc1NCC1CS(=O)(=O)C1] |
| | Intermediate amine |
| | 20 |
| 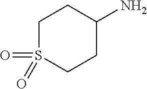 Combi-Blocks | 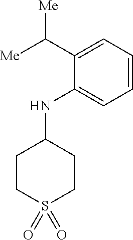 [SMILES: CC(C)c1ccccc1NC1CCS(=O)(=O)CC1] |
| | Intermediate amine |
| | 21 |
| 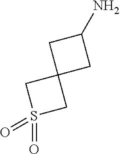 Combi-Blocks | 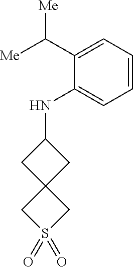 [SMILES: CC(C)c1ccccc1NC1CC2(C1)CS(=O)(=O)C2] |
| | Intermediate amine |
| | 22 |
| 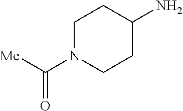 Combi-Blocks | 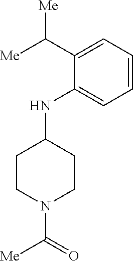 [SMILES: CC(=O)N1CCC(Nc2ccccc2C(C)C)CC1] |
| | Intermediate amine |
| | 23 |
| 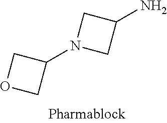 | 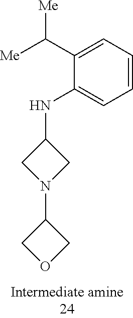 [SMILES: CC(C)c1ccccc1NC1CN(C2COC2)C1] |
| 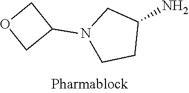 | 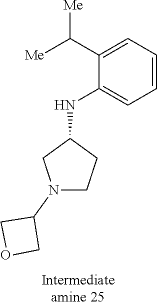 [SMILES: CC(C)c1ccccc1N[C@@H]1CCN(C2COC2)C1] |
| 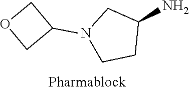 | 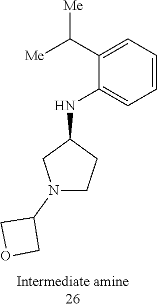 [SMILES: CC(C)c1ccccc1N[C@H]1CCN(C2COC2)C1] |
| 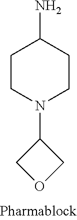 [SMILES: NC1CCN(C2COC2)CC1] | 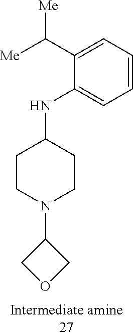 [SMILES: CC(C)c1ccccc1NC1CCN(C2COC2)CC1] |
| 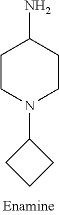 [SMILES: NC1CCN(C2CCC2)CC1] | 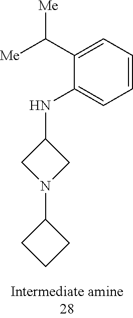 [SMILES: CC(C)c1ccccc1NC1CN(C2CCC2)C1] |
| 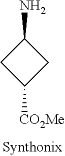 | 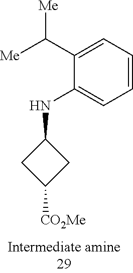 [SMILES: COC(=O)[C@H]1C[C@H](Nc2ccccc2C(C)C)C1] |
| 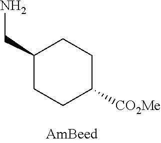 [SMILES: COC(=O)[C@H]1CC[C@H](CN)CC1] | 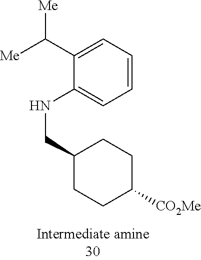 [SMILES: COC(=O)[C@H]1CC[C@H](CNc2ccccc2C(C)C)CC1] |
| 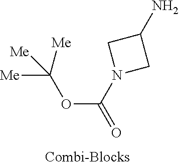 [SMILES: CC(C)(C)OC(=O)N1CC(N)C1] | 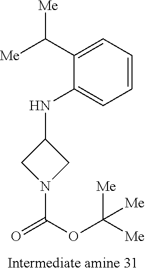 [SMILES: CC(C)c1ccccc1NC1CN(C(=O)OC(C)(C)C)C1] |
| 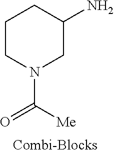 | 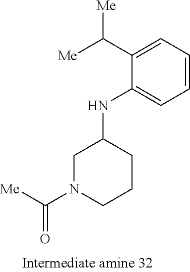 [SMILES: CC(=O)N1CCCC(Nc2ccccc2C(C)C)C1] |
| 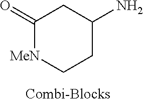 | 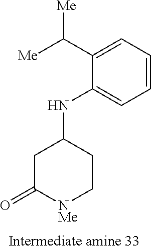 [SMILES: CC(C)c1ccccc1NC1CCN(C)C(=O)C1] |
| 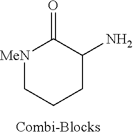 | 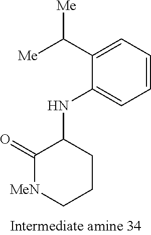 [SMILES: CC(C)c1ccccc1NC1CCCN(C)C1=O] |
| 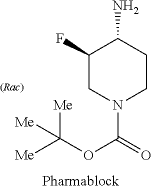 [SMILES: CC(C)(C)OC(=O)N1CC[C@@H](N)[C@H](F)C1] | 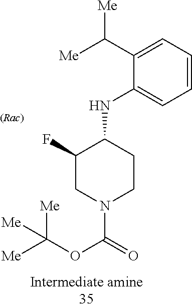 [SMILES: CC(C)c1ccccc1N[C@@H]1CCN(C(=O)OC(C)(C)C)C[C@H]1F] |
| 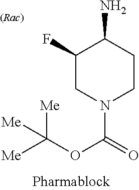 [SMILES: CC(C)(C)OC(=O)N1CC[C@H](N)[C@H](F)C1] | 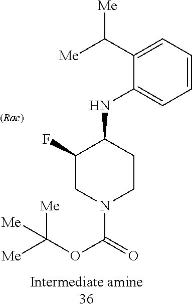 [SMILES: CC(C)c1ccccc1N[C@H]1CCN(C(=O)OC(C)(C)C)C[C@H]1F] |
| 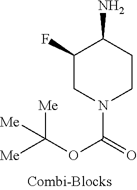 [SMILES: CC(C)(C)OC(=O)N1CC[C@H](N)[C@H](F)C1] | 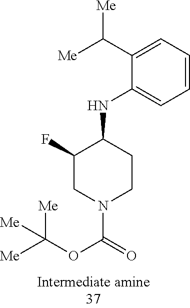 [SMILES: CC(C)c1ccccc1N[C@H]1CCN(C(=O)OC(C)(C)C)C[C@H]1F] |
| 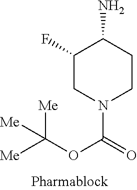 [SMILES: CC(C)(C)OC(=O)N1CC[C@@H](N)[C@@H](F)C1] | 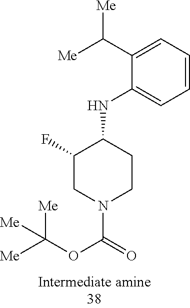 [SMILES: CC(C)c1ccccc1N[C@@H]1CCN(C(=O)OC(C)(C)C)C[C@@H]1F] |
| 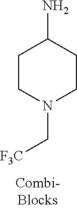 [SMILES: NC1CCN(CC(F)(F)F)CC1] | 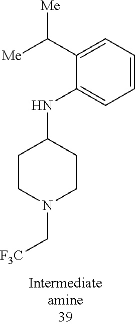 [SMILES: CC(C)c1ccccc1NC1CCN(CC(F)(F)F)CC1] |
| 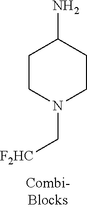 [SMILES: NC1CCN(CC(F)F)CC1] | 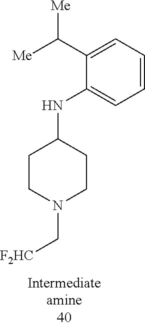 [SMILES: CC(C)c1ccccc1NC1CCN(CC(F)F)CC1] |
| 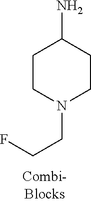 | 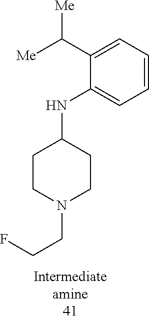 [SMILES: CC(C)c1ccccc1NC1CCN(CCF)CC1] |
| 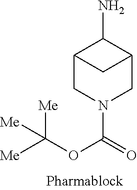 [SMILES: CC(C)(C)OC(=O)N1CC2CC(C1)C2N] | 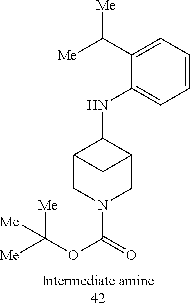 [SMILES: CC(C)c1ccccc1NC1C2CC1CN(C(=O)OC(C)(C)C)C2] |
| 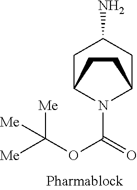 | 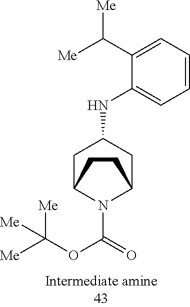 |
| 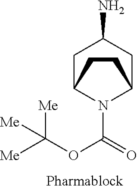 | 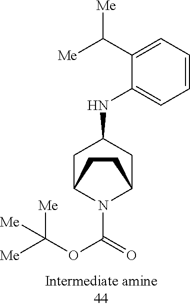 |
| 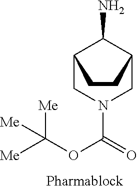 | 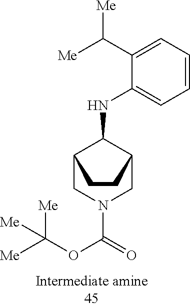 |
| 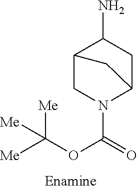 [SMILES: CC(C)(C)OC(=O)N1CC2CC1CC2N] | 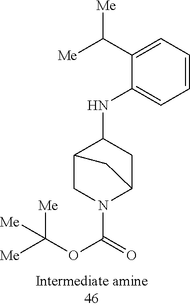 [SMILES: CC(C)c1ccccc1NC1CC2CC1CN2C(=O)OC(C)(C)C] |
| 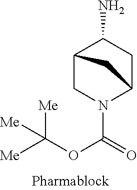 [SMILES: CC(C)(C)OC(=O)N1C[C@H]2C[C@@H]1C[C@H]2N] | 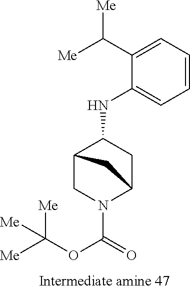 [SMILES: CC(C)c1ccccc1N[C@@H]1C[C@H]2C[C@@H]1CN2C(=O)OC(C)(C)C] |
| 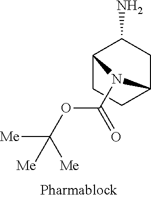 [SMILES: CC(C)(C)OC(=O)N1[C@H]2CC[C@@H]1[C@H](N)C2] | 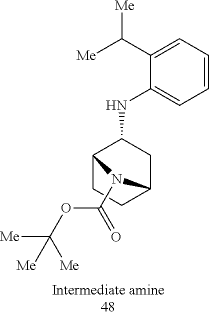 [SMILES: CC(C)c1ccccc1N[C@@H]1C[C@@H]2CC[C@H]1N2C(=O)OC(C)(C)C] |
| 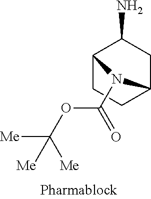 [SMILES: CC(C)(C)OC(=O)N1[C@H]2CC[C@@H]1[C@@H](N)C2] | 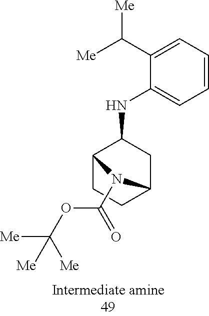 [SMILES: CC(C)c1ccccc1N[C@H]1C[C@@H]2CC[C@H]1N2C(=O)OC(C)(C)C] |
| 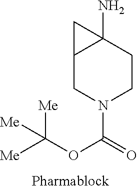 [SMILES: CC(C)(C)OC(=O)N1CCC2(N)CC2C1] | 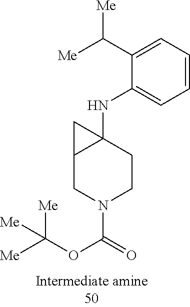 [SMILES: CC(C)c1ccccc1NC12CCN(C(=O)OC(C)(C)C)CC1C2] |
| 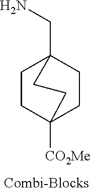 [SMILES: COC(=O)C12CCC(CN)(CC1)CC2] | 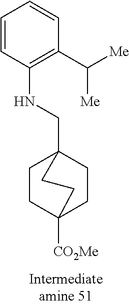 [SMILES: COC(=O)C12CCC(CNc3ccccc3C(C)C)(CC1)CC2] |
| 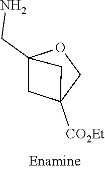 [SMILES: CCOC(=O)C12COC(CN)(C1)C2] | 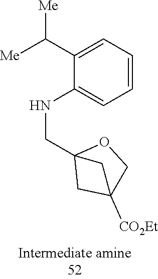 [SMILES: CCOC(=O)C12COC(CNc3ccccc3C(C)C)(C1)C2] |
| 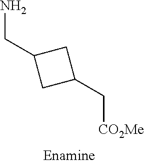 [SMILES: COC(=O)CC1CC(CN)C1] | 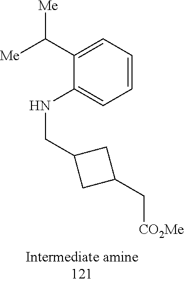 [SMILES: COC(=O)CC1CC(CNc2ccccc2C(C)C)C1] |
| 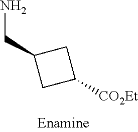 [SMILES: CCOC(=O)[C@H]1C[C@H](CN)C1] | 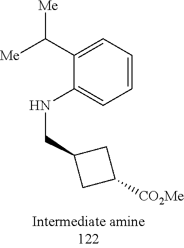 [SMILES: COC(=O)[C@H]1C[C@H](CNc2ccccc2C(C)C)C1] |

### Intermediate amine 53: Preparation of 1-(4-((3-isopropylphenyl)amino)piperidin-1-yl)ethan-1-one

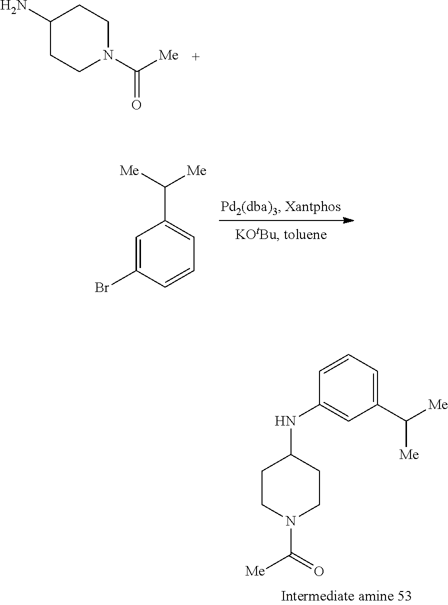 [SMILES: CC(=O)N1CCC(N)CC1 ; CC(C)c1cccc(Br)c1 ; CC(=O)N1CCC(Nc2cccc(C(C)C)c2)CC1]

In a thick-walled glass reaction vessel equipped with a magnetic stirrer and a Teflon screwcap was combined 1-(4-aminopiperidin-1-yl)ethan-1-one (1.2 equiv, Combi-Blocks), 1-bromo-3-isopropylbenzene (1 equiv, Combi-Blocks), 4,5-bis(diphenylphosphino)-9,9-dimethylxanthene (0.2 equiv, Combi-Blocks), and potassium tert-butoxide (4 equiv, Sigma-Aldrich) in toluene (0.19 M). The resulting suspension was deoxygenated via subsurface purging with nitrogen for 10 min and tris(dibenzylidineacetone)dipalladium(0) (0.1 equiv, Sigma-Aldrich) was then added. The reaction vessel was tightly sealed and heated at 100° C. for 2 h. The resulting suspension was cooled to RT, diluted with EtOAc, and washed sequentially with water and brine. The organic extract thus obtained was then dried over Na₂SO₄, filtered, and the filtrate concentrated in vacuo. Purification of the crude product thus obtained by way of column chromatography (SiO₂, gradient elution: DCM→10:1 (v/v) DCM: MeOH) afforded the title compound as a yellow solid (31% yield).

The following amines were prepared in an analogous fashion to Intermediate amine 53, but substituting 1-bromo-3-isopropylbenzene with the requisite, commercially available aryl halide.

| Starting | |
|---|---|
| Material | Product |
| 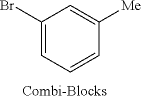 | 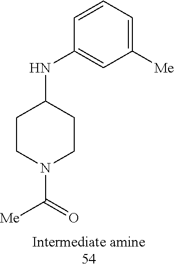 [SMILES: CC(=O)N1CCC(Nc2cccc(C)c2)CC1] |
| 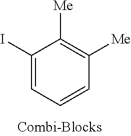 | 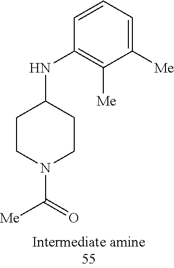 [SMILES: CC(=O)N1CCC(Nc2cccc(C)c2C)CC1] |

### Intermediate amine 56: Preparation of 1-(4-((2-(difluoromethyl)phenyl)amino)piperidin-1-yl)ethan-1-one

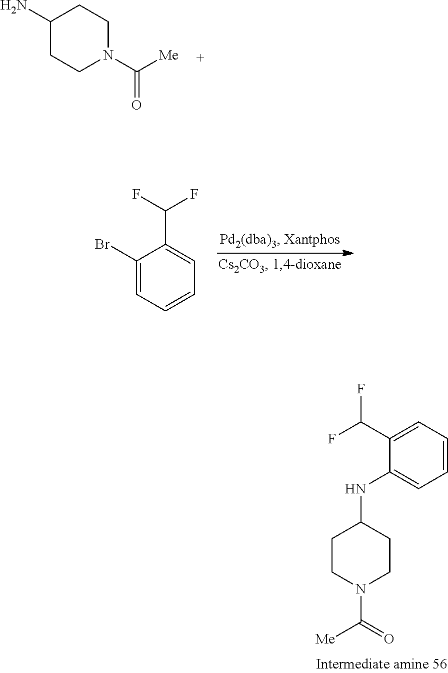 [SMILES: CC(=O)N1CCC(N)CC1 ; FC(F)c1ccccc1Br ; CC(=O)N1CCC(Nc2ccccc2C(F)F)CC1]

In a thick-walled glass reaction vessel equipped with a magnetic stirrer and a Teflon screwcap was combined 1-(4-aminopiperidin-1-yl)ethan-1-one (1.2 equiv, Combi-Blocks), 1-bromo-2-(difluoromethyl)benzene (1 equiv, Combi-Blocks), 4,5-bis(diphenylphosphino)-9,9-dimethylxanthene (0.2 equiv, Combi-Blocks), and cesium carbonate (3 equiv, Sigma-Aldrich) in 1,4-dioxane (0.14 M). The resulting suspension was deoxygenated via subsurface purging with nitrogen for 10 min and tris(dibenzylidineacetone)dipalladium(0) (0.1 equiv, Sigma-Aldrich) was then added. The reaction vessel was tightly sealed and heated at 80° C. for 16 h. The resulting suspension was cooled to RT, diluted with EtOAc, and washed sequentially with water and brine. The organic extract thus obtained was then dried over Na₂SO₄, filtered, and the filtrate concentrated in vacuo. Purification of the crude product thus obtained by way of column chromatography (SiO₂, 3:1 (v/v) EtOAc: Hex) afforded the title compound as a yellow oil (39% yield).

The following amine was prepared in an analogous fashion to Intermediate amine 56, but substituting 1-bromo-2-(difluoromethyl)benzene with the requisite, commercially available aryl halide.

| Starting Material | Product |
|---|---|
| 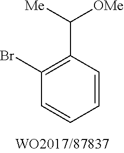 [SMILES: COC(C)c1ccccc1Br] | 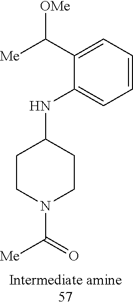 [SMILES: COC(C)c1ccccc1NC1CCN(C(C)=O)CC1] |

### Intermediate amine 58: Preparation of trans-ethyl 4-((2-isopropylphenyl)amino)cyclohexane-1-carboxylate

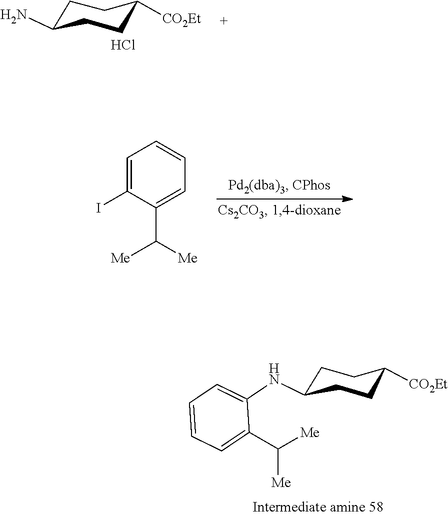

In a thick-walled glass reaction vessel equipped with a magnetic stirrer and a Teflon screwcap was combined trans-ethyl 4-aminocyclohexanecarboxylate hydrochloride (1 equiv, ChemScene), 1-iodo-2-isopropylbenzene (1.4 equiv, Combi-Blocks), tris(dibenzylidineacetone)dipalladium(0) (0.1 equiv, Sigma-Aldrich), 2-cyclohexylphosphino-2′,6′-bis(N,N-dimethylamino)biphenyl (0.2 equiv, Combi-Blocks), and cesium carbonate (5 equiv, Sigma-Aldrich) in 1,4-dioxane (0.12 M). The resulting purple suspension was then deoxygenated via subsurface purging with nitrogen for 10 min before the reaction vessel was tightly sealed and heated at 90° C. for 48 h. The resulting orange, brown suspension was cooled to RT, diluted with tert-butyl methyl ether, and washed sequentially with water and brine. The organic extract thus obtained was then dried over MgSO₄, treated with charcoal, filtered through a bed of celite, and the filtrate concentrated in vacuo. Purification of the crude product thus obtained by way of column chromatography (SiO₂, gradient elution: Hex→4:1 (v/v) Hex: EtOAc) afforded the title compound as a golden yellow oil (67% yield).

The following amines were prepared in an analogous fashion to Intermediate amine 58, but substituting trans-ethyl 4-aminocyclohexanecarboxylate hydrochloride with the requisite, commercially available amine.

| Starting | Product |
|---|---|
| 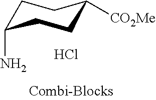 | 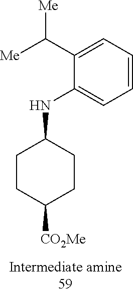 [SMILES: COC(=O)[C@H]1CC[C@@H](Nc2ccccc2C(C)C)CC1] |
| 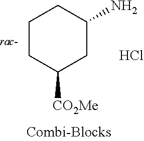 [SMILES: COC(=O)[C@H]1CCC[C@H](N)C1 ; Cl] | 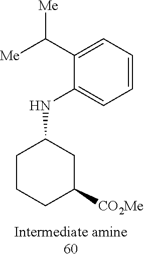 [SMILES: COC(=O)[C@H]1CCC[C@H](Nc2ccccc2C(C)C)C1] |
| 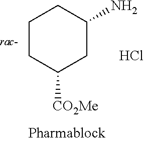 [SMILES: COC(=O)[C@@H]1CCC[C@H](N)C1 ; Cl] | 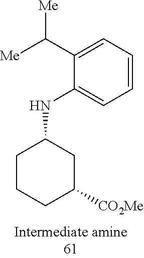 [SMILES: COC(=O)[C@@H]1CCC[C@H](Nc2ccccc2C(C)C)C1] |
| 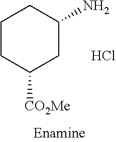 [SMILES: COC(=O)[C@@H]1CCC[C@H](N)C1 ; Cl] | 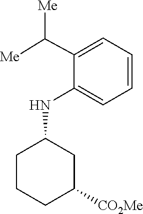 [SMILES: COC(=O)[C@@H]1CCC[C@H](Nc2ccccc2C(C)C)C1] |
| | Intermediate amine 152 |
| 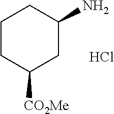 [SMILES: COC(=O)[C@H]1CCC[C@@H](N)C1 ; Cl] Enamine | 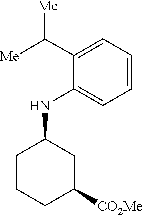 [SMILES: COC(=O)[C@H]1CCC[C@@H](Nc2ccccc2C(C)C)C1] |
| | Intermediate amine 153 |
| 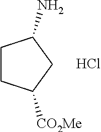 Ambeed | 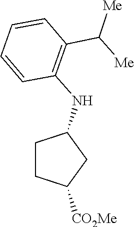 [SMILES: COC(=O)[C@@H]1CC[C@H](Nc2ccccc2C(C)C)C1] |
| | Intermediate amine 156 |
| 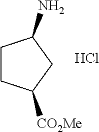 Ambeed | 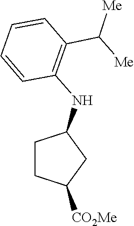 [SMILES: COC(=O)[C@H]1CC[C@@H](Nc2ccccc2C(C)C)C1] |
| | Intermediate amine 157 |
| 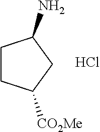 Ambeed | 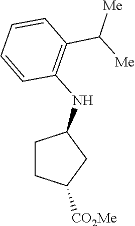 [SMILES: COC(=O)[C@@H]1CC[C@@H](Nc2ccccc2C(C)C)C1] |
| | Intermediate amine 158 |
| 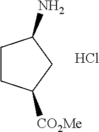 Ambeed | 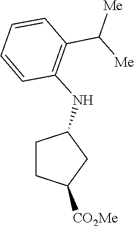 [SMILES: COC(=O)[C@H]1CC[C@H](Nc2ccccc2C(C)C)C1] |
| | Intermediate amine 159 |
| 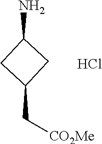 Enamine | 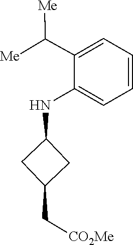 [SMILES: COC(=O)C[C@H]1C[C@@H](Nc2ccccc2C(C)C)C1] |
| | Intermediate amine 160 |
| 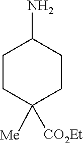 [SMILES: CCOC(=O)C1(C)CCC(N)CC1] Pharmablock | 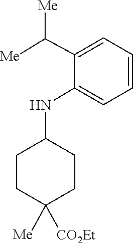 [SMILES: CCOC(=O)C1(C)CCC(Nc2ccccc2C(C)C)CC1] |
| | Intermediate amine 161 |
| 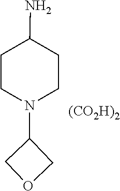 Pharmablock | 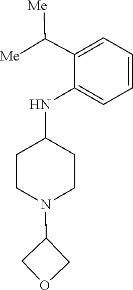 [SMILES: CC(C)c1ccccc1NC1CCN(C2COC2)CC1] |
| | Intermediate amine 162 |

### Intermediate amine 62: Preparation of methyl 2-(3-((2-isopropylphenyl)amino)cyclobutyl)acetate

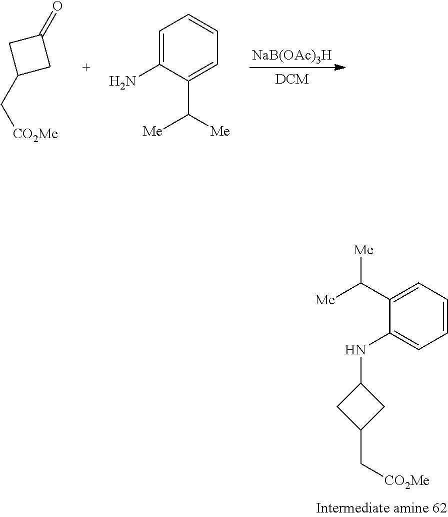 [SMILES: COC(=O)CC1CC(=O)C1 ; CC(C)c1ccccc1N ; COC(=O)CC1CC(Nc2ccccc2C(C)C)C1 ; "NaB(OAc)3H"]

In a dried, round-bottom flask equipped with a magnetic stirrer was combined 2-isopropylaniline (1 equiv, Combi-Blocks) and methyl 2-(3-oxocyclobutyl)acetate (1.1 equiv, Enamine) in dichloromethane (0.86 M). To this mixture was then added sodium triacetoxyborohydride (1.2 equiv, Sigma-Aldrich) in one rapid portion, and the resulting suspension was allowed to stir at RT for 30 min. The volatiles were then removed in vacuo and the crude product thus obtained was purified further by way of column chromatography (SiO₂, gradient elution: Hex→1:1 (v/v) Hex: EtOAc) to afford the title compound as an inseparable mixture of cis- and trans-isomers (55% yield).

The following amines were prepared in an analogous fashion to Intermediate amine 62, but substituting methyl 2-(3-oxocyclobutyl)acetate with the requisite, commercially available ketone/aldehyde. For the synthesis of Intermediate amine 65, Intermediate amine 70, Intermediate amine 71, and Intermediate amine 73, acetic acid (0.1 equiv, Sigma-Aldrich) and freshly activated 4 Å molecular sieves were also included as additives. For the preparation of Intermediate amine 67 and Intermediate amine 68, these two diastereomers were separable by column chromatography. Similarly, for the preparation of Intermediate amine 140 and Intermediate amine 141, these two diastereomers were separable by column chromatography. For the synthesis of Intermediate amine 146, 2-isopropylaniline (1 equiv) was also substituted with 2-tert-butylaniline (1 equiv, TCI). For the synthesis of Intermediate amine 147, 2-isopropylaniline (1 equiv) was also substituted with 2-cyclopropylaniline (1 equiv, Sigma-Aldrich).

| Starting | |
|---|---|
| Material | Product |
| 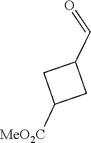 Combi-Blocks | 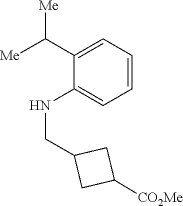 [SMILES: COC(=O)C1CC(CNc2ccccc2C(C)C)C1] |
| | Intermediate amine 63 |
| 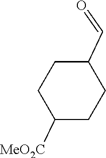 [SMILES: COC(=O)C1CCC(C=O)CC1] AmBeed | 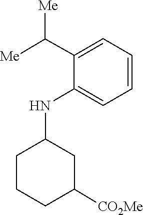 [SMILES: COC(=O)C1CCCC(Nc2ccccc2C(C)C)C1] |
| | Intermediate amine 64 |
| 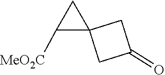 [SMILES: COC(=O)C1CC12CC(=O)C2] Enamine | 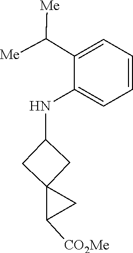 [SMILES: COC(=O)C1CC12CC(Nc1ccccc1C(C)C)C2] |
| | Intermediate amine 65 |
| 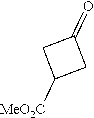 Pharmablock | 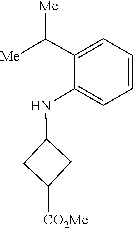 [SMILES: COC(=O)C1CC(Nc2ccccc2C(C)C)C1] |
| | Intermediate amine 66 |
| 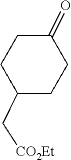 [SMILES: CCOC(=O)CC1CCC(=O)CC1] Combi-Blocks | 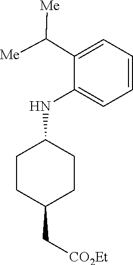 [SMILES: CCOC(=O)C[C@H]1CC[C@H](Nc2ccccc2C(C)C)CC1] |
| | Intermediate amine 67 |
| 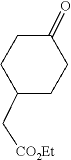 [SMILES: CCOC(=O)CC1CCC(=O)CC1] Combi-Blocks | 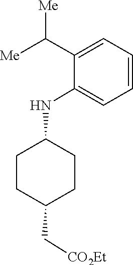 [SMILES: CCOC(=O)C[C@H]1CC[C@@H](Nc2ccccc2C(C)C)CC1] |
| | Intermediate amine 68 |
| 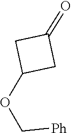 [SMILES: O=C1CC(OCc2ccccc2)C1] Pharmablock | 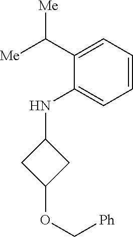 [SMILES: CC(C)c1ccccc1NC1CC(OCc2ccccc2)C1] |
| | Intermediate amine 69 |
| 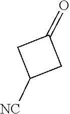 Synthonix | 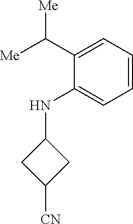 [SMILES: CC(C)c1ccccc1NC1CC(C#N)C1] |
| | Intermediate amine 70 |
| 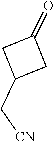 Combi-Blocks | 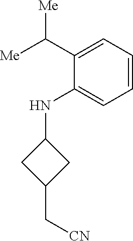 [SMILES: CC(C)c1ccccc1NC1CC(CC#N)C1] |
| | Intermediate amine 71 |
| 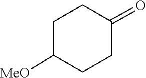 Combi-Blocks | 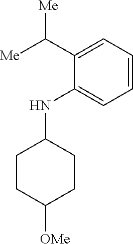 [SMILES: COC1CCC(Nc2ccccc2C(C)C)CC1] |
| | Intermediate amine 72 |
| 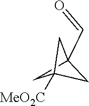 [SMILES: COC(=O)C12CC(C=O)(C1)C2] AChemBlock | 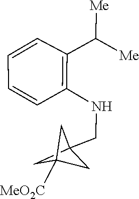 [SMILES: COC(=O)C12CC(CNc3ccccc3C(C)C)(C1)C2] |
| | Intermediate amine 73 |
| 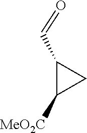 AstaTech | 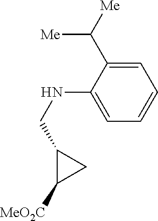 [SMILES: COC(=O)[C@@H]1C[C@H]1CNc1ccccc1C(C)C] |
| | Intermediate amine 123 |
| 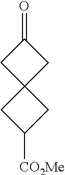 [SMILES: COC(=O)C1CC2(CC(=O)C2)C1] Combi-Blocks | 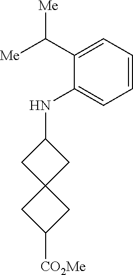 [SMILES: COC(=O)C1CC2(CC(Nc3ccccc3C(C)C)C2)C1] |
| | Intermediate amine 124 |
| 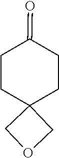 Combi-Blocks | 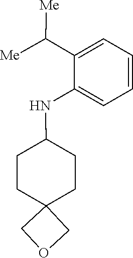 [SMILES: CC(C)c1ccccc1NC1CCC2(CC1)COC2] |
| | Intermediate amine 126 |
| 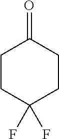 Combi-Blocks | 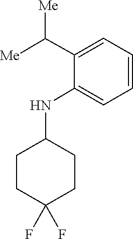 [SMILES: CC(C)c1ccccc1NC1CCC(F)(F)CC1] |
| | Intermediate amine |
| | 127 |
| 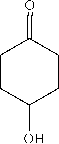 Combi-Blocks | 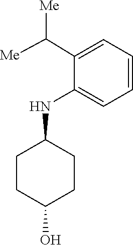 [SMILES: CC(C)c1ccccc1N[C@H]1CC[C@H](O)CC1] |
| | Intermediate amine |
| | 129 |
| 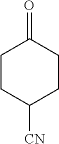 Combi-Blocks | 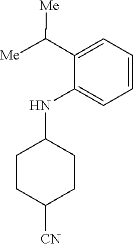 [SMILES: CC(C)c1ccccc1NC1CCC(C#N)CC1] |
| | Intermediate amine |
| | 130 |
| 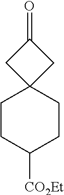 [SMILES: CCOC(=O)C1CCC2(CC1)CC(=O)C2] Pharmablock | 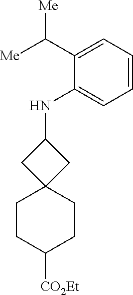 [SMILES: CCOC(=O)C1CCC2(CC1)CC(Nc1ccccc1C(C)C)C2] |
| | Intermediate amine |
| | 131 |
| 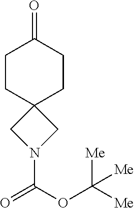 [SMILES: CC(C)(C)OC(=O)N1CC2(CCC(=O)CC2)C1] Combi-Blocks | 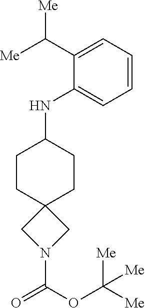 [SMILES: CC(C)c1ccccc1NC1CCC2(CC1)CN(C(=O)OC(C)(C)C)C2] |
| | Intermediate amine |
| | 132 |
| 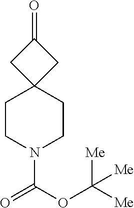 [SMILES: CC(C)(C)OC(=O)N1CCC2(CC1)CC(=O)C2] Combi-Blocks | 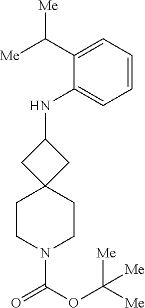 [SMILES: CC(C)c1ccccc1NC1CC2(CCN(C(=O)OC(C)(C)C)CC2)C1] |
| | Intermediate amine 134 |
| 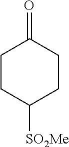 [SMILES: CS(=O)(=O)C1CCC(=O)CC1] Combi-Blocks | 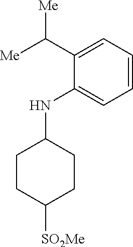 [SMILES: CC(C)c1ccccc1NC1CCC(S(C)(=O)=O)CC1] |
| | Intermediate amine 138 |
| 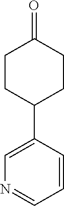 [SMILES: O=C1CCC(c2cccnc2)CC1] Asta Tech | 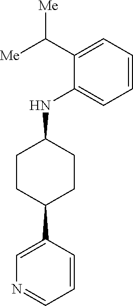 [SMILES: CC(C)c1ccccc1N[C@H]1CC[C@@H](c2cccnc2)CC1] |
| | Intermediate amine 140 |
| 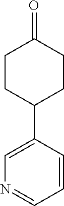 [SMILES: O=C1CCC(c2cccnc2)CC1] AstaTech | 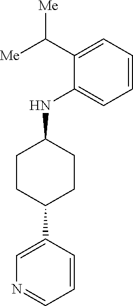 [SMILES: CC(C)c1ccccc1N[C@H]1CC[C@H](c2cccnc2)CC1] |
| | Intermediate amine 141 |
| 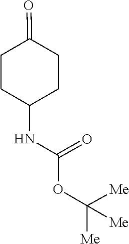 [SMILES: CC(C)(C)OC(=O)NC1CCC(=O)CC1] Combi-Blocks | 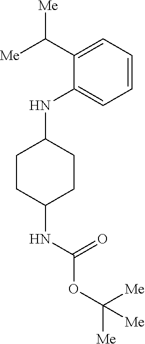 [SMILES: CC(C)c1ccccc1NC1CCC(NC(=O)OC(C)(C)C)CC1] |
| | Intermediate amine 144 |
| 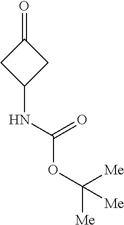 [SMILES: CC(C)(C)OC(=O)NC1CC(=O)C1] Combi-Blocks | 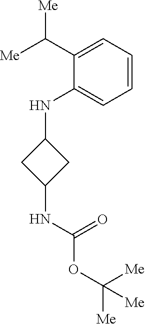 [SMILES: CC(C)c1ccccc1NC1CC(NC(=O)OC(C)(C)C)C1] |
| | Intermediate amine 145 |
| 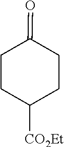 [SMILES: CCOC(=O)C1CCC(=O)CC1] Combi-Blocks | 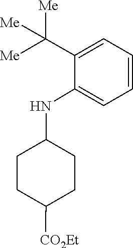 [SMILES: CCOC(=O)C1CCC(Nc2ccccc2C(C)(C)C)CC1] |
| | Intermediate amine 146 |
| 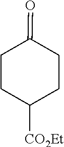 [SMILES: CCOC(=O)C1CCC(=O)CC1] Combi-Blocks | 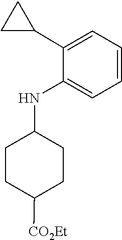 [SMILES: CCOC(=O)C1CCC(Nc2ccccc2C2CC2)CC1] |
| | Intermediate amine |
| | 147 |
| 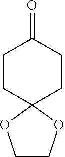 [SMILES: O=C1CCC2(CC1)OCCO2] Combi-Blocks | 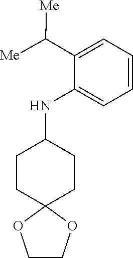 [SMILES: CC(C)c1ccccc1NC1CCC2(CC1)OCCO2] |
| | Intermediate amine |
| | 148 |

### Intermediate amine 149: Preparation of methyl (1r,4r)-4-((2-(2-hydroxypropan-2-yl)phenyl)amino)cyclohexane-1-carboxylate

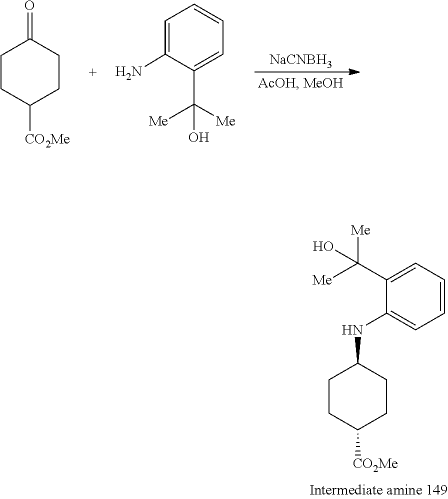

In a dried, round-bottom flask equipped with a magnetic stirrer was combined 2-(2-aminophenyl)propan-2-ol (1 equiv, Combi-Blocks) and methyl 4-oxocyclohexane-1-carboxylate (1.4 equiv, Combi-Blocks) in a 5:1 (v/v) solution of methanol and glacial acetic acid (0.14 M). To this mixture was then added sodium cyanoborohydride (1.6 equiv, Sigma-Aldrich) in one rapid portion, and the resulting suspension was allowed to stir at RT for 1 h. The volatiles were then removed in vacuo and the resulting residue was partitioned between water and EtOAc. The organic layer was separated and washed sequentially with saturated aq. NaHCO₃ and brine, dried over MgSO₄, filtered, and the filtrate concentrated in vacuo. The crude product thus obtained was purified further by way of column chromatography (SiO₂, gradient elution: Hex→4:1 (v/v) Hex: EtOAc) to afford the title compound as the slower eluting isomer (22% yield).

### Intermediate amine 74: Preparation of 1-(4-((3-isopropylpyridin-2-yl)amino)piperidin-1-yl)ethan-1-one

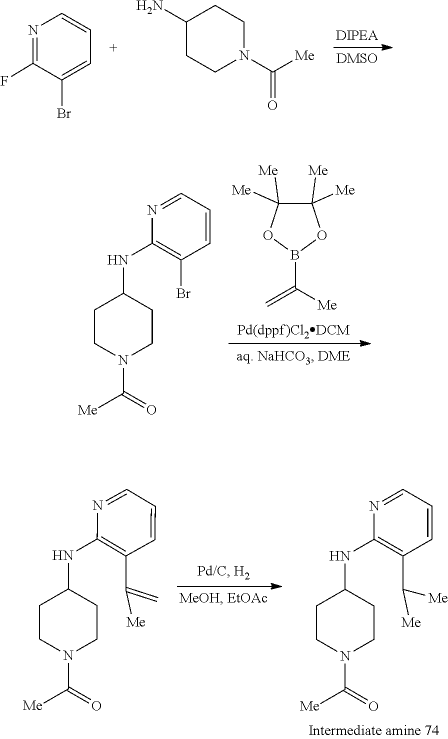

Step 1: In a dried, round-bottom flask equipped with a magnetic stirrer was combined 3-bromo-2-fluoropyridine (1 equiv, Combi-Blocks), 1-(4-aminopiperidin-1-yl)ethan-1-one (1.3 equiv, Combi-Blocks), and N,N-diisopropylethylamine (2.5 equiv, Sigma-Aldrich) in dimethylsulfoxide (0.33 M). The resulting solution was then heated at 120° C. for 10 h. After cooling to RT, the reaction was quenched with water and extracted with EtOAc. The combined organic extracts were then washed further with water and brine, dried over MgSO₄, filtered, and the filtrate concentrated in vacuo. Purification of the crude product thus obtained by way of column chromatography (SiO₂, gradient elution: 9:1 (v/v) Hex: EtOAc+10% MeOH→EtOAc+10% MeOH) afforded 1-(4-((3-bromopyridin-2-yl)amino)piperidin-1-yl)ethan-1-one as a tan solid (41% yield).

Step 2: In a thick-walled glass reaction vessel equipped with a magnetic stirrer and a Teflon screwcap was combined 1-(4-((3-bromopyridin-2-yl)amino)piperidin-1-yl)ethan-1-one (1 equiv) from the previous step, 4,4,5,5-tetramethyl-2-(prop-1-en-2-yl)-1,3,2-dioxaborolane (3 equiv, Frontier Scientific), [1,1′-bis(diphenylphosphino)ferrocene]dichloropalladium(II) dichloromethane complex (0.1 equiv, Sigma-Aldrich), and sodium bicarbonate (3 equiv, Alfa Aesar) in a 2:1 (v/v) solution of 1,2-dimethoxyethane and water (0.1 M). The resulting mixture was deoxygenated via subsurface purging with nitrogen for 10 min before the reaction vessel was tightly sealed and heated at 85° C. for 10 h. The now dark brown suspension was cooled to RT, quenched with water, and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: 9:1 (v/v) Hex: EtOAc+10% MeOH→EtOAc+10% MeOH) afforded 1-(4-((3-(prop-1-en-2-yl)pyridin-2-yl)amino)piperidin-1-yl)ethan-1-one as a tan solid (83% yield).

Step 3: In a dried, round-bottom flask equipped with a magnetic stirrer was dissolved 1-(4-((3-(prop-i-en-2-yl)pyridin-2-yl)amino)piperidin-1-yl)ethan-1-one (1 equiv) from the previous step in a 1:1 (v/v) solution of ethyl acetate and methanol (0.06 M). The resulting solution was then deoxygenated via subsurface purging with nitrogen for 10 min before palladium (10% w/w over activated carbon, dry, 0.1 equiv, Sigma-Aldrich) was added in one rapid portion. The resulting black suspension was then subsurface purged with hydrogen for 10 min before it was stirred under a static hydrogen atmosphere (maintained with a balloon) at RT for 30 min. The reaction was subsequently quenched with dichloromethane and filtered through a bed of dichloromethane-wetted celite. Concentration of the filtrate in vacuo afforded the title compound as a foam (96% yield).

### Intermediate amine 75: Preparation of 1-(4-((2-isopropylpyridin-3-yl)amino)piperidin-1-yl)ethan-1-one

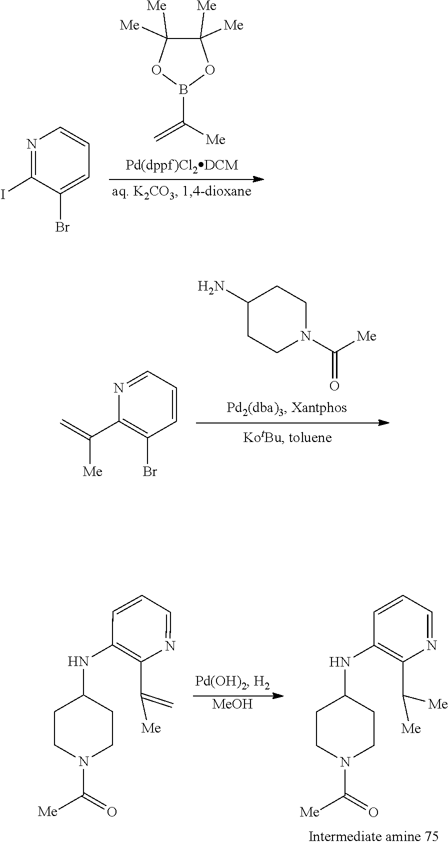

Step 1: In a thick-walled glass reaction vessel equipped with a magnetic stirrer and a Teflon screwcap was combined 3-bromo-2-iodopyridine (1 equiv, Combi-Blocks), 4,4,5,5-tetramethyl-2-(prop-1-en-2-yl)-1,3,2-dioxaborolane (2.5 equiv, Frontier Scientific), potassium carbonate (3 equiv, Sigma-Aldrich), and [1,1′-bis(diphenylphosphino)ferrocene]dichloropalladium(II) dichloromethane complex (0.1 equiv, Sigma-Aldrich) in a 5:1 (v/v) solution of 1,4-dioxane and water (0.12 M). The resulting mixture was deoxygenated via subsurface purging with nitrogen for 10 min before the reaction vessel was tightly sealed and heated at 60° C. for 2 h. The now dark brown suspension was cooled to RT, diluted further with water, and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: DCM→9:1 (v/v) DCM: MeOH) afforded 3-bromo-2-(prop-1-en-2-yl)pyridine as a red solid (67% yield).

Step 2: In a thick-walled glass reaction vessel equipped with a magnetic stirrer and a Teflon screwcap was combined 3-bromo-2-(prop-1-en-2-yl)pyridine (1 equiv) from the previous step, 1-(4-aminopiperidin-1-yl)ethan-1-one (1.2 equiv, Combi-Blocks), 4,5-bis(diphenylphosphino)-9,9-dimethylxanthene (0.2 equiv, Combi-Blocks), and potassium tert-butoxide (2.5 equiv, Sigma-Aldrich) in toluene (0.15 M). The resulting solution was deoxygenated via subsurface purging with nitrogen for 10 min and tris(dibenzylidineacetone)dipalladium(0) (0.10 equiv, Sigma-Aldrich) was then added. The reaction vessel was tightly sealed and heated at 100° C. for 2 h. The resulting dark brown suspension was cooled to RT, diluted with EtOAc, and washed sequentially with water and brine. The organic extract thus obtained was then dried over Na₂SO₄, filtered, and the filtrate concentrated in vacuo. Purification of the crude product thus obtained by way of column chromatography (SiO₂, gradient elution: DCM→9:1 (v/v) DCM: MeOH) afforded 1-(4-((2-(prop-1-en-2-yl)pyridin-3-yl)amino)piperidin-1-yl)ethan-1-one as a red solid (61% yield).

Step 3: In a dried, round-bottom flask equipped with a magnetic stirrer was dissolved 1-(4-((2-(prop-1-en-2-yl)pyridin-3-yl)amino)piperidin-1-yl)ethan-1-one (1 equiv) from the previous step in methanol (0.041 M). The resulting solution was then deoxygenated via subsurface purging with nitrogen for 10 min before palladium(II) hydroxide (10% w/w over activated carbon, dry, 0.1 equiv, Sigma-Aldrich) was added in one rapid portion. The resulting black suspension was then subsurface purged with hydrogen for 10 min before it was stirred under a static hydrogen atmosphere (maintained with a balloon) at RT for 1 h. The reaction was subsequently quenched with dichloromethane and filtered through a bed of dichloromethane-wetted celite. Concentration of the filtrate in vacuo and purification of the crude product thus obtained by way of column chromatography (SiO₂, gradient elution: 9:1 (v/v) Hex: EtOAc+10% MeOH→EtOAc+10% MeOH) afforded the title compound as a red oil (74% yield).

The following amine was prepared in an analogous fashion to Intermediate amine 75, but substituting 3-bromo-2-iodopyridine with the requisite, commercially available (hetero)aryl halide.

| Starting Material | Product |
|---|---|
| 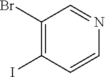 Combi-Blocks | 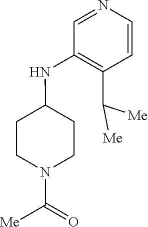 [SMILES: CC(=O)N1CCC(Nc2cnccc2C(C)C)CC1] |
| | Intermediate amine 76 |

### Intermediate amine 77: Preparation of 1-(4-((2-(prop-1-en-2-yl)phenyl)amino)piperidin-1-yl)ethan-1-one

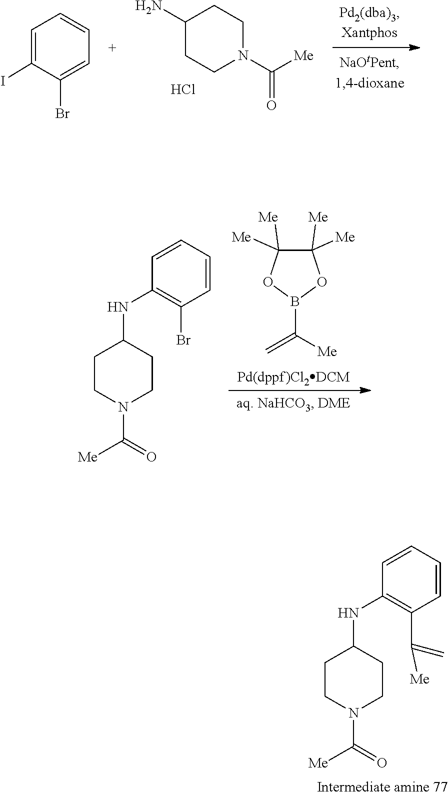

Step 1: In a thick-walled glass reaction vessel equipped with a magnetic stirrer and a Teflon screwcap was combined 1-(4-aminopiperidin-1-yl)ethan-1-one hydrochloride (1.1 equiv, Combi-Blocks), 1-bromo-2-iodobenzene (1 equiv, Combi-Blocks), 4,5-bis(diphenylphosphino)-9,9-dimethylxanthene (0.1 equiv, Combi-Blocks), and sodium tert-pentoxide (3.5 equiv, Sigma-Aldrich) in 1,4-dioxane (0.17 M). The resulting yellow solution was deoxygenated via subsurface purging with nitrogen for 10 min and tris(dibenzylidineacetone)dipalladium(0) (0.05 equiv, Sigma-Aldrich) was then added. The reaction vessel was tightly sealed and heated at 100° C. for 5 h. The resulting dark brown suspension was cooled to RT, diluted with EtOAc, and washed sequentially with water and brine. The organic extract thus obtained was then dried over MgSO₄, filtered, and the filtrate concentrated in vacuo. Purification of the crude product thus obtained by way of column chromatography (SiO₂, gradient elution: Hex→4:1 (v/v) Hex: EtOAc) afforded 1-(4-((2-bromophenyl)amino)piperidin-1-yl)ethan-1-one as a yellow oil (77% yield).

Step 2: In a thick-walled glass reaction vessel equipped with a magnetic stirrer and a Teflon screwcap was combined 1-(4-((2-bromophenyl)amino)piperidin-1-yl)ethan-1-one (1 equiv) from the previous step, 4,4,5,5-tetramethyl-2-(prop-1-en-2-yl)-1,3,2-dioxaborolane (3.7 equiv, Frontier Scientific), [1,1′-bis(diphenylphosphino)ferrocene]dichloropalladium(II) dichloromethane complex (0.09 equiv, Sigma-Aldrich), and sodium bicarbonate (2.8 equiv, Alfa Aesar) in a 2:1 (v/v) solution of 1,2-dimethoxyethane and water (0.09 M). The resulting mixture was deoxygenated via subsurface purging with nitrogen for 10 min before the reaction vessel was tightly sealed and heated at 85° C. for 18 h. The now dark brown suspension was cooled to RT, quenched with water, and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: 9:1 (v/v) Hex: EtOAc+10% MeOH→EtOAc+10% MeOH) afforded the title compound as a tan solid (67% yield).

### Intermediate amine 78: Preparation of 4-((2-isopropylphenyl)amino)pyrrolidin-2-one

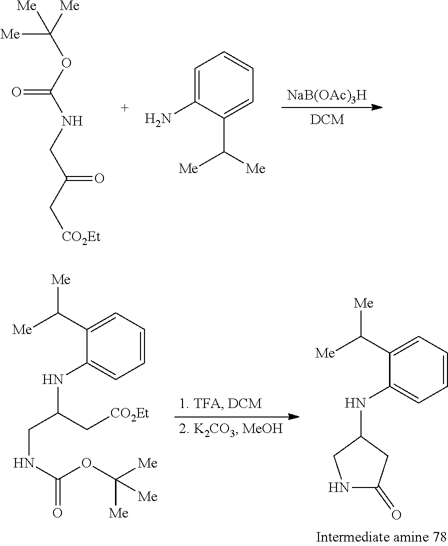

Step 1: In a dried, round-bottom flask equipped with a magnetic stirrer was combined 2-isopropylaniline (1 equiv, Combi-Blocks) and ethyl 4-((tert-butoxycarbonyl)amino)-3-oxobutanoate (1.1 equiv, Ambeed) in dichloromethane (0.49 M). To this mixture was then added sodium triacetoxyborohydride (1.2 equiv, Sigma-Aldrich) in one rapid portion, and the resulting suspension was stirred at RT for 10 min. The volatiles were then removed in vacuo and the crude product thus obtained was purified further by way of column chromatography (SiO₂, gradient elution: Hex→4:1 (v/v) Hex: EtOAc) to afford ethyl 4-((tert-butoxycarbonyl)amino)-3-((2-isopropylphenyl)amino)butanoate as an orange oil (8.4% yield).

Step 2: In a dried, round-bottom flask equipped with a magnetic stirrer was dissolved ethyl 4-((tert-butoxycarbonyl)amino)-3-((2-isopropylphenyl)amino)butanoate (1 equiv) from the previous step in dichloromethane (0.062 M). To this yellow solution was then added trifluoroacetic acid (10 equiv, Sigma-Aldrich), and the resulting mixture was stirred at RT for 30 min. The volatiles were then removed in vacuo and further azeotroped with toluene. The resulting residue and potassium carbonate (4 equiv, Sigma-Aldrich) were then combined in a 5:1 (v/v) solution of acetonitrile and methanol (0.21 M), and heated at 68° C. for 20 min. The volatiles were then removed in vacuo and the resulting residue was partitioned between water and EtOAc. The aqueous layer was separated and back extracted with EtOAc. The combined organic extracts were washed further with water and brine, dried over Na₂SO₄, and filtered. Concentration of the filtrate thus obtained in vacuo afforded the title compound as a tan solid (>99% yield).

### Intermediate amine 79: Preparation of 1-(3-((2-isopropylphenyl)amino)azetidin-1-yl)ethan-1-one

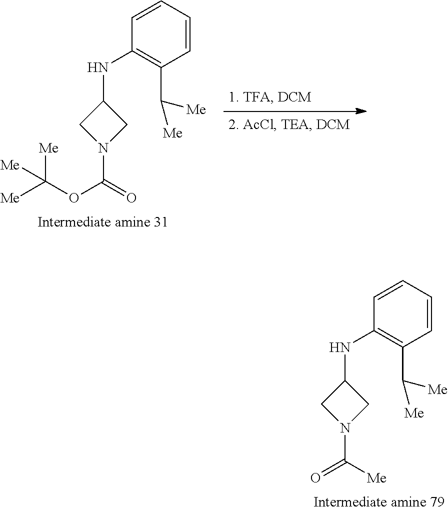 [SMILES: CC(C)c1ccccc1NC1CN(C(=O)OC(C)(C)C)C1 ; CC(=O)N1CC(Nc2ccccc2C(C)C)C1]

In a round-bottom flask equipped with a magnetic stirrer was dissolved Intermediate amine 31 (1 equiv) in dichloromethane (0.22 M). To this solution was then added trifluoroacetic acid (150 equiv, Sigma-Aldrich) and the resulting reaction mixture was stirred at RT for 2 h. The volatiles were then removed in vacuo via sequential azeotropic distillation with toluene and heptane. The residue thus obtained was then partitioned between EtOAc and saturated aq. NaHCO₃. The aqueous layer was separated and back extracted with EtOAc. The combined organic extracts were washed further with water and brine, dried over Na₂SO₄, filtered, and the filtrate concentrated in vacuo. The crude N-(2-isopropylphenyl)azetidin-3-amine thus obtained was then re-taken up in dichloromethane (0.44 M), and added sequentially triethylamine (2.5 equiv, Sigma-Aldrich) and acetyl chloride (1.5 equiv, Sigma-Aldrich). After 2 h of stirring at RT, the reaction was quenched with water and extracted with DCM. The combined organic extracts were washed further with brine, dried over Na₂SO₄, filtered, and the filtrate concentrated in vacuo. Purification of the crude product thus obtained by way of column chromatography (SiO₂, 1:1 (v/v) Hex: EtOAc) afforded the title compound as a yellow solid (4300 yield).

The following amines were prepared in an analogous fashion to Intermediate amine 79, but substituting Intermediate amine 31, with the requisite starting amine.

| Starting Amine | Product |
|---|---|
| 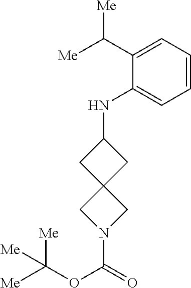 [SMILES: CC(C)c1ccccc1NC1CC2(C1)CN(C(=O)OC(C)(C)C)C2] | 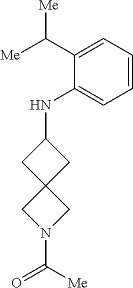 [SMILES: CC(=O)N1CC2(CC(Nc3ccccc3C(C)C)C2)C1] |
| Intermediate | Intermediate |
| amine 11 | amine 80 |
| 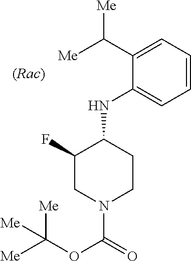 [SMILES: CC(C)c1ccccc1N[C@@H]1CCN(C(=O)OC(C)(C)C)C[C@H]1F] | 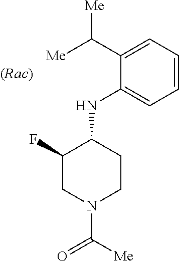 [SMILES: CC(=O)N1CC[C@@H](Nc2ccccc2C(C)C)[C@H](F)C1] |
| Intermediate amine | Intermediate amine |
| 35 | 81 |
| 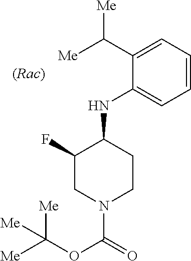 [SMILES: CC(C)c1ccccc1N[C@H]1CCN(C(=O)OC(C)(C)C)C[C@H]1F] | 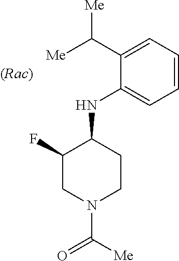 [SMILES: CC(=O)N1CC[C@H](Nc2ccccc2C(C)C)[C@H](F)C1] |
| Intermediate | Intermediate |
| amine 36 | amine 82 |
| 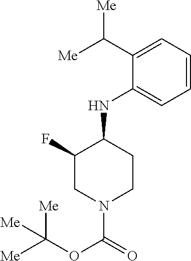 [SMILES: CC(C)c1ccccc1N[C@H]1CCN(C(=O)OC(C)(C)C)C[C@H]1F] | 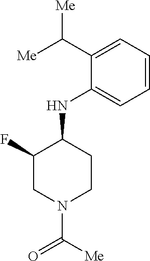 [SMILES: CC(=O)N1CC[C@H](Nc2ccccc2C(C)C)[C@H](F)C1] |
| Intermediate amine | Intermediate amine |
| 37 | 83 |
| 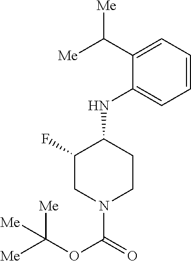 [SMILES: CC(C)c1ccccc1N[C@@H]1CCN(C(=O)OC(C)(C)C)C[C@@H]1F] | 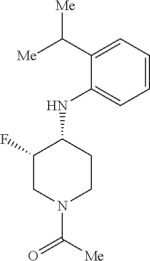 [SMILES: CC(=O)N1CC[C@@H](Nc2ccccc2C(C)C)[C@@H](F)C1] |
| Intermediate | Intermediate |
| amine 38 | amine 84 |
| 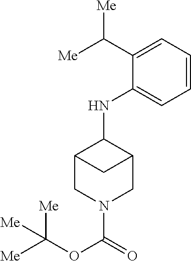 [SMILES: CC(C)c1ccccc1NC1C2CC1CN(C(=O)OC(C)(C)C)C2] | 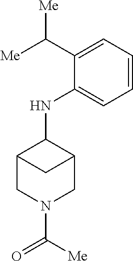 [SMILES: CC(=O)N1CC2CC(C1)C2Nc1ccccc1C(C)C] |
| Intermediate amine | Intermediate amine |
| 42 | 85 |
| 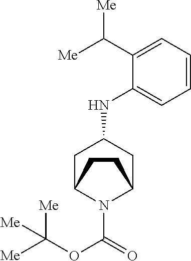 | 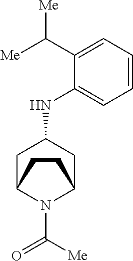 |
| Intermediate | Intermediate |
| amine 43 | amine 86 |
| 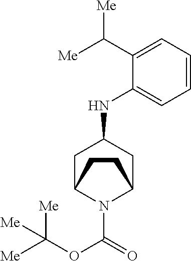 | 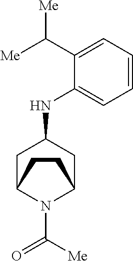 |
| Intermediate amine | Intermediate amine |
| 44 | 87 |
| 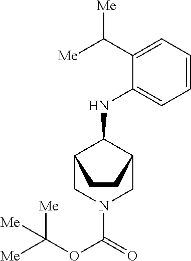 | 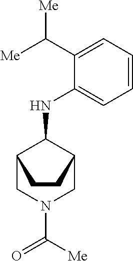 |
| Intermediate | Intermediate |
| amine 45 | amine 88 |
| 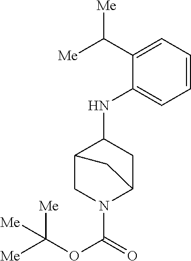 [SMILES: CC(C)c1ccccc1NC1CC2CC1CN2C(=O)OC(C)(C)C] | 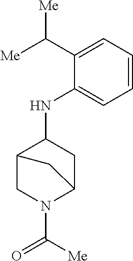 [SMILES: CC(=O)N1CC2CC1CC2Nc1ccccc1C(C)C] |
| Intermediate amine | Intermediate amine |
| 46 | 89 |
| 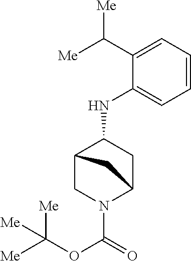 [SMILES: CC(C)c1ccccc1N[C@@H]1C[C@H]2C[C@@H]1CN2C(=O)OC(C)(C)C] | 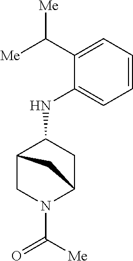 [SMILES: CC(=O)N1C[C@H]2C[C@@H]1C[C@H]2Nc1ccccc1C(C)C] |
| Intermediate | Intermediate |
| amine 47 | amine 90 |
| 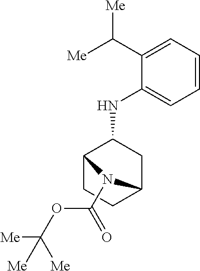 [SMILES: CC(C)c1ccccc1N[C@@H]1C[C@@H]2CC[C@H]1N2C(=O)OC(C)(C)C] | 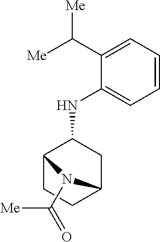 [SMILES: CC(=O)N1[C@H]2CC[C@@H]1[C@H](Nc1ccccc1C(C)C)C2] Intermediate amine 91 |
| Intermediate amine 48 | |
| 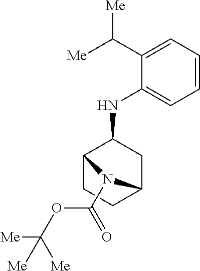 [SMILES: CC(C)c1ccccc1N[C@H]1C[C@@H]2CC[C@H]1N2C(=O)OC(C)(C)C] | 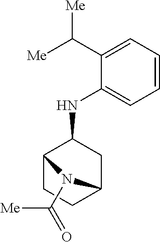 [SMILES: CC(=O)N1[C@H]2CC[C@@H]1[C@@H](Nc1ccccc1C(C)C)C2] Intermediate amine 92 |
| Intermediate | |
| amine 49 | |
| 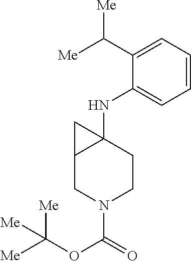 [SMILES: CC(C)c1ccccc1NC12CCN(C(=O)OC(C)(C)C)CC1C2] | 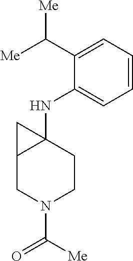 [SMILES: CC(=O)N1CCC2(Nc3ccccc3C(C)C)CC2C1] |
| Intermediate amine | Intermediate amine |
| 50 | 93 |
| 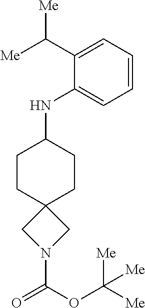 [SMILES: CC(C)c1ccccc1NC1CCC2(CC1)CN(C(=O)OC(C)(C)C)C2] | 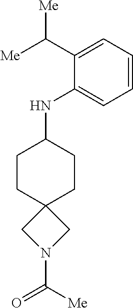 [SMILES: CC(=O)N1CC2(CCC(Nc3ccccc3C(C)C)CC2)C1] |
| Intermediate | Intermediate |
| amine 132 | amine 133 |
| 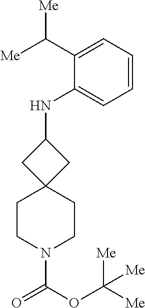 [SMILES: CC(C)c1ccccc1NC1CC2(CCN(C(=O)OC(C)(C)C)CC2)C1] | 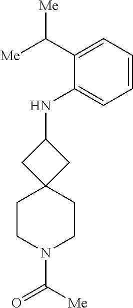 [SMILES: CC(=O)N1CCC2(CC1)CC(Nc1ccccc1C(C)C)C2] |
| Intermediate amine | Intermediate amine |
| 134 | 135 |

### Intermediate amine 94: Preparation of ethyl 1-(2-(3-((2-isopropylphenyl)amino)azetidin-1-yl)ethyl)cyclobutane-1-carboxylate

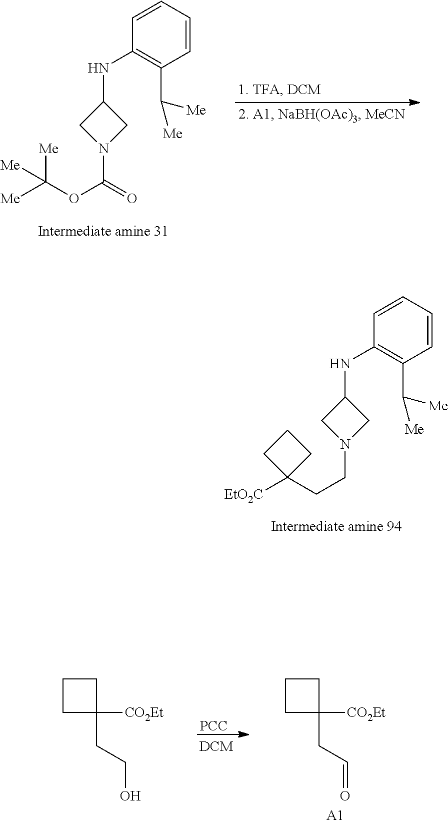

Step 1, preparation of A1: In a dried, round-bottom flask equipped with a magnetic stirrer was added pyridinium chlorochromate (1.5 equiv, Sigma-Aldrich) portionwise to a dichloromethane solution (0.29 M) of ethyl 1-(2-hydroxyethyl)cyclobutane-1-carboxylate (1 equiv, Pharmablock). The resulting mixture was then stirred at RT for 16 h before it was quenched with the careful addition of water. The aqueous layer was separated and back extracted with DCM. The combined organic extracts were washed further with water and brine, dried over Na₂SO₄, filtered, and the filtrate concentrated in vacuo. Purification of the crude product thus obtained by way of column chromatography (SiO₂, 1:1 (v/v) Hex: EtOAc) afforded ethyl 1-(2-oxoethyl)cyclobutane-1-carboxylate as a yellow oil (64% yield).

Step 2: In a round-bottom flask equipped with a magnetic stirrer was dissolved Intermediate amine 31 (1 equiv) in dichloromethane (0.22 M). To this solution was then added trifluoroacetic acid (150 equiv, Sigma-Aldrich) and the resulting reaction mixture was stirred at RT for 2 h. The volatiles were then removed in vacuo via sequential azeotropic distillation with toluene and heptane. The residue thus obtained was then partitioned between EtOAc and saturated aq. NaHCO₃. The aqueous layer was separated and back extracted with EtOAc. The combined organic extracts were washed further with water and brine, dried over Na₂SO₄, filtered, and the filtrate concentrated in vacuo. The crude N-(2-isopropylphenyl)azetidin-3-amine thus obtained was then combined with ethyl 1-(2-oxoethyl)cyclobutane-1-carboxylate (1.1 equiv) from the previous step in acetonitrile (0.053 M). To this mixture was then added sodium triacetoxyborohydride (3 equiv, Sigma-Aldrich) in one rapid portion, and the resulting suspension stirred at RT for 16 h. The volatiles were then removed in vacuo and the crude product thus obtained was purified further by way of column chromatography (SiO₂, gradient elution: Hex→1:1 (v/v) Hex: EtOAc) to afford the title compound as a yellow oil (48% yield).

### Intermediate amine 125: Preparation of methyl 2,2-difluoro-3-(((2-isopropylphenyl)amino)methyl)bicyclo[1.1.1]pentane-1-carboxylate

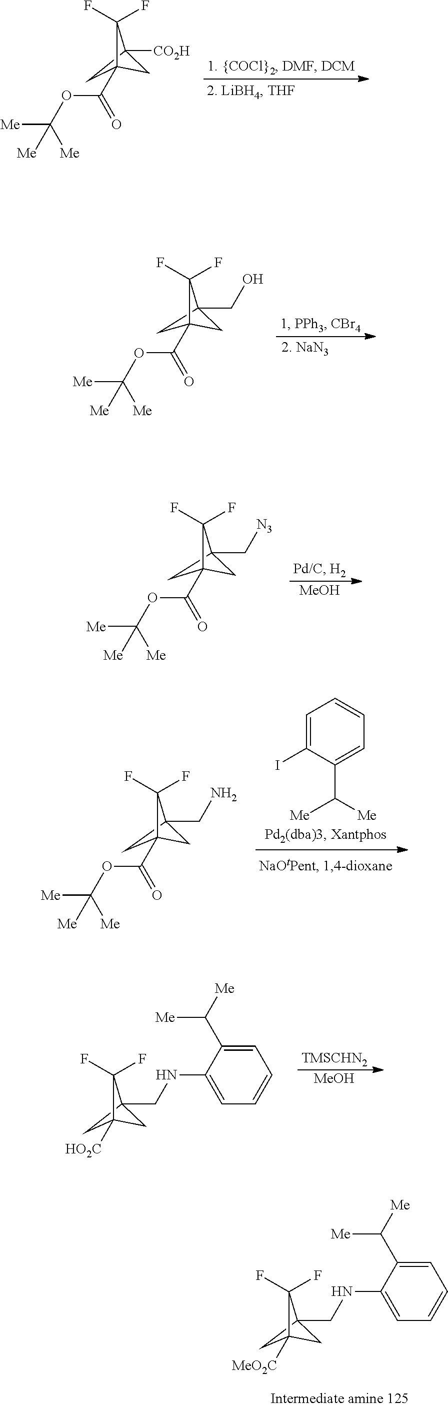

Step 1: In a dried, round-bottom flask equipped with a magnetic stirrer was combined 3-(tert-butoxycarbonyl)-2,2-difluorobicyclo[1.1.1]pentane-1-carboxylic acid (1 equiv, Enamine) and anhydrous DMF (a few drops) in anhydrous dichloromethane (0.1 M). The resulting solution was then cooled to 0° C. before oxalyl chloride (2.5 equiv, Sigma-Aldrich) was added neat and dropwise over a period of 5 min. The resulting mixture was stirred at 0° C. for 5 min and then at RT for an additional 45 min. The volatiles were removed in vacuo and the resulting residue was then taken up in anhydrous THE (0.17 M). The reaction mixture was cooled again to 0° C. before lithium borohydride (1 M solution in THF, 1 equiv, Sigma-Aldrich) was added dropwise over a period of 10 min. The resulting mixture was stirred at 0° C. for 5 min and then at RT for an additional 45 min. Finally, the reaction was carefully quenched at 0° C. with the dropwise addition of 1 M aq. HCl and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→2:3 (v/v) Hex: EtOAc) afforded tert-butyl 2,2-difluoro-3-(hydroxymethyl)bicyclo[1.1.1]pentane-1-carboxylate as a colorless oil (87% yield).

Step 2: In a dried, round-bottom flask equipped with a magnetic stirrer was combined tert-butyl 2,2-difluoro-3-(hydroxymethyl)bicyclo[1.1.1]pentane-1-carboxylate (1 equiv) from the previous step and carbon tetrabromide (3 equiv, Sigma-Aldrich) in anhydrous DMF (0.17 M). To this solution was then added triphenylphosphine (3 equiv, Combi-Blocks) in one rapid portion and the resulting mixture was stirred at RT for 70 min. Finally, sodium azide (7 equiv, Sigma-Aldrich) was added in one rapid portion and the resulting mixture was stirred at RT for 16 h. The reaction mixture was then diluted with water and extracted with tert-butyl methyl ether. The combined organic extracts were washed further with water, saturated aq. NaHCO₃ and brine, dried over MgSO₄, filtered, and the filtrate concentrated in vacuo. Purification of the crude product thus obtained by way of column chromatography (SiO₂, gradient elution: Hex→6:1 (v/v) Hex: EtOAc) afforded tert-butyl 3-(azidomethyl)-2,2-difluorobicyclo[1.1.1]pentane-1-carboxylate as a colorless oil (77% yield).

Step 3: In a dried, round-bottom flask equipped with a magnetic stirrer was dissolved tert-butyl 3-(azidomethyl)-2,2-difluorobicyclo[1.1.1]pentane-1-carboxylate (1 equiv) from the previous step in methanol (0.11 M). The resulting solution was then deoxygenated via subsurface purging with nitrogen for 10 min before palladium (10% w/w over activated carbon, dry, 0.1 equiv, Sigma-Aldrich) was added in one rapid portion. The resulting black suspension was then subsurface purged with hydrogen for 10 min before it was stirred under a static hydrogen atmosphere at RT for 30 min. The reaction was subsequently quenched with dichloromethane and filtered through a bed of dichloromethane-wetted celite. Concentration of the filtrate thus obtained in vacuo afforded tert-butyl 3-(aminomethyl)-2,2-difluorobicyclo[1.1.1]pentane-1-carboxylate as a colorless oil (79% yield).

Step 4: In a thick-walled glass reaction vessel equipped with a magnetic stirrer and a Teflon screwcap was combined tert-butyl 3-(aminomethyl)-2,2-difluorobicyclo[1.1.1]pentane-1-carboxylate (1 equiv) from the previous step, 1-iodo-2-isopropylbenzene (1 equiv, Combi-Blocks), 4,5-bis(diphenylphosphino)-9,9-dimethylxanthene (0.2 equiv, Combi-Blocks), and sodium tert-pentoxide (3 equiv, Sigma-Aldrich) in 1,4-dioxane (0.11 M). The resulting yellow solution was deoxygenated via subsurface purging with nitrogen for 10 min and tris(dibenzylidineacetone)dipalladium(0) (0.1 equiv, Sigma-Aldrich) was then added. The reaction vessel was tightly sealed and heated at 100° C. for 40 min. The resulting dark brown suspension was cooled to RT, diluted with EtOAc, and washed sequentially with 1 M aq. HCl, water, and brine. The organic extract thus obtained was then dried over MgSO₄, filtered, and the filtrate concentrated in vacuo. Purification of the crude product thus obtained by way of reverse-phase column chromatography (C₁₈, gradient elution: 9:1 (v/v) H₂O: MeCN+0.1% formic acid→MeCN+0.1% formic acid) afforded 2,2-difluoro-3-(((2-isopropylphenyl)amino)methyl)bicyclo[1.1.1]pentane-1-carboxylic acid as a white solid (74% yield).

Step 5: In a dried, round-bottom flask equipped with a magnetic stirrer was dissolved 2,2-difluoro-3-(((2-isopropylphenyl)amino)methyl)bicyclo[1.1.1]pentane-1-carboxylic acid (1 equiv) from the previous step in a 10:1 (v/v) solution of toluene and methanol (0.065 M). To this solution was then added (trimethylsilyl)diazomethane (2 M solution in diethyl ether, 1.5 equiv, Sigma-Aldrich) dropwise over a period of 5 min. The resulting yellow solution was stirred at RT for an additional 10 min before the reaction was quenched with the addition of glacial acetic acid. The reaction mixture was then diluted with water and extracted with EtOAc. The combined organic extracts were washed further with water, saturated aq. NaHCO₃ and brine, dried over MgSO₄, filtered, and the filtrate concentrated in vacuo. Purification of the crude product thus obtained by way of column chromatography (SiO₂, gradient elution: Hex→4:1 (v/v) Hex: EtOAc) afforded the title compound as a white foam (66% yield).

### Intermediate amine 128: Preparation of 2-((1s,4s)-4-((2-isopropylphenyl)amino)cyclohexyl)propan-2-ol

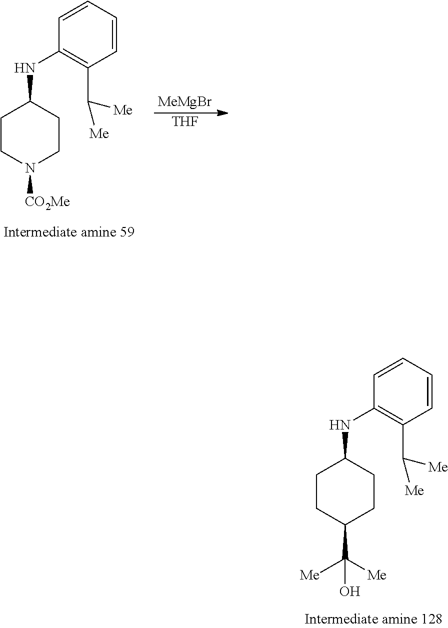

In a dried, round-bottom flask equipped with a magnetic stirrer was dissolved Intermediate amine 59 (1 equiv) in anhydrous THF (0.11 M). To this solution was then added, at 0° C., methylmagnesium bromide (3 M solution in diethyl ether, 3.3 equiv, Sigma-Aldrich) dropwise over 5 min. The resulting suspension was stirred first at 0° C. for 30 min and then at RT for 3 h. The reaction was then carefully quenched with the sequential addition of water and saturated aq. NH₄Cl. The resulting suspension was vigorously stirred at RT for 30 min and then extracted with EtOAc. The combined organic extracts were dried over MgSO₄, filtered, and the filtrate concentrated in vacuo. Purification of the crude product thus obtained by way of column chromatography (SiO₂, gradient elution: Hex→4:1 (v/v) Hex: EtOAc) afforded the title compound as a colorless oil (34% yield).

### Intermediate amine 143: Preparation of methyl (1r,4r)-4-((2-(propan-2-yl-1,1,1,3,3,3-d₆)phenyl)amino)cyclohexane-1-carboxylate

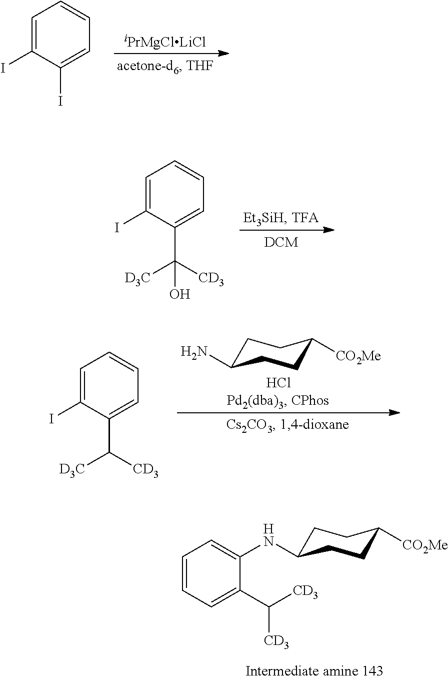

Step 1: In a dried, round-bottom flask equipped with a magnetic stirrer was dissolved 1,2-diiodobenzene (1 equiv, Combi-Blocks) in anhydrous THE (0.11 M). To this solution was then added, at −20° C., Turbo Grignard (1.3 M solution in THF, 1.2 equiv, Sigma-Aldrich) dropwise over 5 min, and the resulting solution was stirred at −20° C. for 20 min. Finally, acetone-d₆ (3.4 equiv, Sigma-Aldrich) was added neat and dropwise over 5 min and the resulting mixture was allowed to warm slowly to RT over 3 h. After cooling to 0° C., the reaction was carefully quenched with the sequential addition of water and saturated aq. NH₄Cl. The resulting suspension was vigorously stirred at RT for 30 min, and then extracted with EtOAc. The combined organic extracts were dried over MgSO₄, filtered, and the filtrate concentrated in vacuo. Purification of the crude product thus obtained first by way of regular phase column chromatography (SiO₂, gradient elution: Hex→3:7 (v/v) Hex: EtOAc), and then by reverse-phase column chromatography (C₁₈, gradient elution: 9:1 (v/v) H₂O: MeCN+0.1% formic acid→MeCN+0.1% formic acid) afforded 2-(2-iodophenyl)propan-1,1,1,3,3,3-d-2-ol as a colorless oil (14% yield).

Step 2: In a dried, round-bottom flask equipped with a magnetic stirrer was combined 2-(2-iodophenyl)propan-1,1,1,3,3,3-d-2-ol (1 equiv) from the previous step and trifluoroacetic acid (10 equiv, Sigma-Aldrich) in dichloromethane (0.11 M). To this solution was then added triethylsilane (1.5 equiv, Sigma-Aldrich) neat and dropwise over 5 min, and the resulting solution was stirred at RT for 40 min. The volatiles were then removed in vacuo and the crude product thus obtained was purified by way of reverse-phase column chromatography (C₁₈, gradient elution: 9:1 (v/v) H₂O: MeCN+0.1% formic acid→MeCN+0.1% formic acid) to afford 1-iodo-2-(propan-2-yl-1,1,1,3,3,3-d₆)benzene as a colorless oil (45% yield).

Step 3: In a thick-walled glass reaction vessel equipped with a magnetic stirrer and a Teflon screwcap was combined 1-iodo-2-(propan-2-yl-1,1,1,3,3,3-d₆)benzene (1 equiv) from the previous step, trans-methyl 4-aminocyclohexanecarboxylate hydrochloride (1 equiv, Combi-Blocks), tris(dibenzylidineacetone)dipalladium(0) (0.1 equiv, Sigma-Aldrich), 2-cyclohexylphosphino-2′,6′-bis(N,N-dimethylamino)biphenyl (0.2 equiv, Combi-Blocks), and cesium carbonate (4 equiv, Sigma-Aldrich) in 1,4-dioxane (0.10 M). The resulting purple suspension was then deoxygenated via subsurface purging with nitrogen for 10 min before the reaction vessel was tightly sealed and heated at 90° C. for 48 h. The resulting orange, brown suspension was cooled to RT, diluted with tert-butyl methyl ether, and washed sequentially with water and brine. The organic extract thus obtained was dried over MgSO₄, treated with charcoal, filtered through a bed of celite, and the filtrate concentrated in vacuo. Purification of the crude product thus obtained by way of column chromatography (SiO₂, gradient elution: Hex→1:1 (v/v) Hex: EtOAc) afforded the title compound as a golden yellow oil (41% yield).

### Intermediate amine 150: Preparation of methyl (1r,4r)-4-((2-(1-((tert-butyldimethylsilyl)oxy)propan-2-yl)phenyl)amino)cyclohexane-1-carboxylate

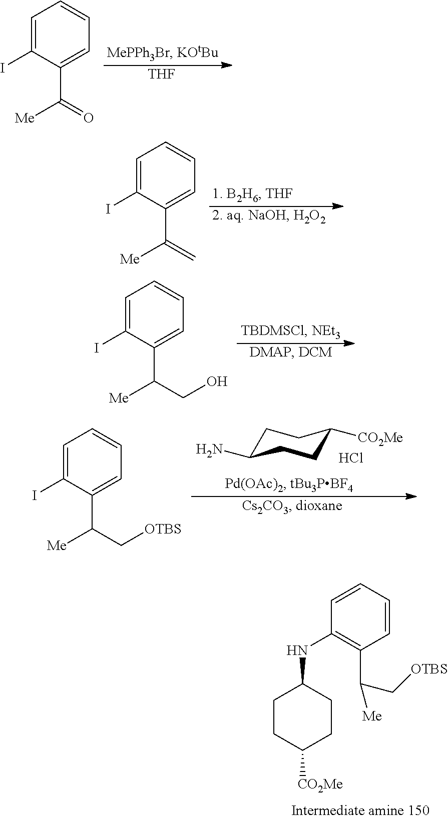

Step 1: In a dried, round-bottom flask equipped with a magnetic stirrer was combined, at 0° C., methyltriphenylphosphonium bromide (1.2 equiv, Sigma-Aldrich) and potassium tert-butoxide (1.2 equiv, Sigma-Aldrich) in anhydrous THF (0.76 M). The resulting bright yellow suspension was stirred at 0° C. for 30 min after which time 2′-iodoacetophenone (1 equiv, TCI) was added as a solution in THE (1.3 M) dropwise over a period of 5 min. The resulting suspension was then allowed to warm slowly to RT over 16 h. The insolubles were removed via vacuum filtration and washed further with diethyl ether. The filtrate thus obtained was concentrated in vacuo. The resulting residue was then purified by way of column chromatography (SiO₂, Hex) to afford 1-iodo-2-(prop-1-en-2-yl)benzene as a colorless oil (42% yield).

Step 2: In a dried, round-bottom flask equipped with a magnetic stirrer was dissolved 1-iodo-2-(prop-1-en-2-yl)benzene (1 equiv) from the previous step in anhydrous THF (0.20 M). To this solution was then added, at 0° C., borane (1 M solution in THF, 1.1 equiv, Sigma-Aldrich) dropwise over a period of 5 min. The resulting solution was stirred at 0° C. for 90 min before the reaction was quenched with the sequential and dropwise addition of NaOH (2.5 M solution in water, 3.5 equiv) and hydrogen peroxide (30% w/w solution in water, Sigma-Aldrich). After the completion of addition, the cooling bath was removed, and the biphasic solution was stirred vigorously at RT for 1 h. The reaction mixture was then diluted with water and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→2:3 (v/v) Hex: EtOAc) afforded 2-(2-iodophenyl)propan-1-ol as a colorless oil (77% yield).

Step 3: In a dried, round-bottom flask equipped with a magnetic stirrer was combined 2-(2-iodophenyl)propan-1-ol (1 equiv) from the previous step, tert-butyldimethylsilyl chloride (1.2 equiv, Sigma-Aldrich), triethylamine (1.6 equiv, Sigma-Aldrich), and 4-dimethylaminopyridine (0.1 equiv, Sigma-Aldrich) in dichloromethane (0.22 M). The resulting mixture was stirred at RT for 16 h and then quenched with the addition of water. The aqueous layer was separated and back extracted with dichloromethane. The combined organic extracts were washed further with water and brine, dried over MgSO₄, and filtered. Concentration of the filtrate in vacuo furnished tert-butyl(2-(2-iodophenyl)propoxy)dimethylsilane as a colorless oil.

Step 4: In a thick-walled glass reaction vessel equipped with a magnetic stirrer and a Teflon screwcap was combined tert-butyl(2-(2-iodophenyl)propoxy)dimethylsilane (1 equiv) from the previous step, trans-methyl 4-aminocyclohexanecarboxylate hydrochloride (1.1 equiv, Combi-Blocks), palladium(II) acetate (0.06 equiv, Sigma-Aldrich), tri-tert-butylphosphonium tetrafluoroborate (0.12 equiv, Combi-Blocks), and cesium carbonate (4 equiv, Sigma-Aldrich) in 1,4-dioxane (0.10 M). The resulting yellow suspension was then deoxygenated via subsurface purging with nitrogen for 10 min before the reaction vessel was tightly sealed and heated at 95° C. for 18 h. The resulting brown suspension was cooled to RT, diluted with tert-butyl methyl ether, and washed sequentially with water and brine. The organic extract thus obtained was then dried over MgSO₄, treated with charcoal, filtered through a bed of celite, and the filtrate concentrated in vacuo. Purification of the crude product thus obtained by way of column chromatography (SiO₂, gradient elution: Hex→1:1 (v/v) Hex: EtOAc) afforded the title compound as a golden yellow oil (50% yield over two steps).

### Intermediate amine 95: Preparation of 2-(difluoromethoxy)-6-methoxypyridin-3-amine

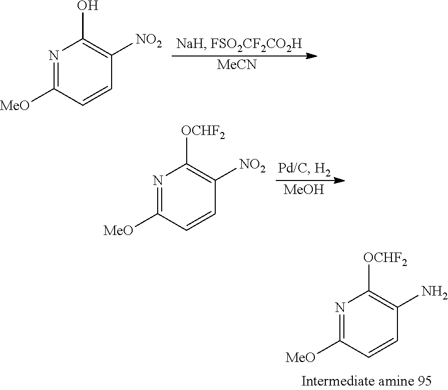 [SMILES: "MeCN" ; "MeOH" ; COc1ccc([N+](=O)[O-])c(O)n1 ; COc1ccc([N+](=O)[O-])c(OC(F)F)n1 ; COc1ccc(N)c(OC(F)F)n1]

Step 1: In a dried, round-bottom flask equipped with a magnetic stirrer was suspended 6-methoxy-3-nitropyridin-2-ol (1 equiv, Combi-Blocks) in acetonitrile (0.10 M). To this was then added sodium hydride (60% w/w dispersion in paraffin oil, 2.8 equiv, Sigma-Aldrich) in one rapid portion and the resulting mixture was stirred at RT for 10 min to afford a brownish, yellow suspension. Then, 2,2-difluoro-2-(fluorosulfonyl)acetic acid (1.8 equiv, Sigma-Aldrich) was added neat and dropwise over a period of 5 min, during which time a mild exotherm was observed. After 16 h of stirring, another aliquot of 2,2-difluoro-2-(fluorosulfonyl)acetic acid (1.8 equiv, Sigma-Aldrich) was added neat and dropwise over a period of 5 min. After another 48 h of stirring at RT, the crude reaction mixture was carefully quenched with water, and then diluted with a 1:1 (v/v) solution of ethyl acetate and hexanes. The organic layer was then separated and washed sequentially with saturated aq. NaHCO₃, water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 2-(difluoromethoxy)-6-methoxy-3-nitropyridine as a yellow solid (75% yield).

Step 2: In a dried, round-bottom flask equipped with a magnetic stirrer was dissolved 2-(difluoromethoxy)-6-methoxy-3-nitropyridine (1 equiv) from the previous step in methanol (0.17 M). The resulting yellow solution was then deoxygenated via subsurface purging with nitrogen for 10 min before palladium (10% w/w over activated carbon, dry, 0.08 equiv, Sigma-Aldrich) was added in one rapid portion. The resulting black suspension was then subsurface purged with hydrogen for 10 min before it was stirred under a static hydrogen atmosphere (maintained with a balloon) at RT for 90 min. The reaction was subsequently diluted with EtOAc and filtered through a bed of dichloromethane-wetted celite. The insolubles were washed further with EtOAc. Concentration of the filtrate thus obtained in vacuo afforded the title compound as a reddish, brown solid (>99% yield).

The following amines were prepared in an analogous fashion to Intermediate amine 95, but substituting 6-methoxy-3-nitropyridin-2-ol in step 1 with the requisite, commercially available (hetero)aryl alcohol.

| Starting | |
|---|---|
| Material | Product |
| 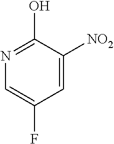 [SMILES: O=[N+]([O-])c1cc(F)cnc1O] | 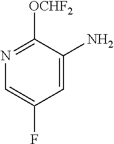 [SMILES: Nc1cc(F)cnc1OC(F)F] |
| Combi-Blocks | Intermediate amine 96 |
| 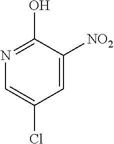 [SMILES: O=[N+]([O-])c1cc(Cl)cnc1O] | 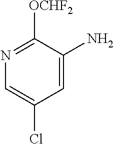 [SMILES: Nc1cc(Cl)cnc1OC(F)F] |
| Combi-Blocks | Intermediate amine 97 |

### Intermediate amine 98: Preparation of 4-(difluoromethoxy)-6-methylpyridin-3-amine

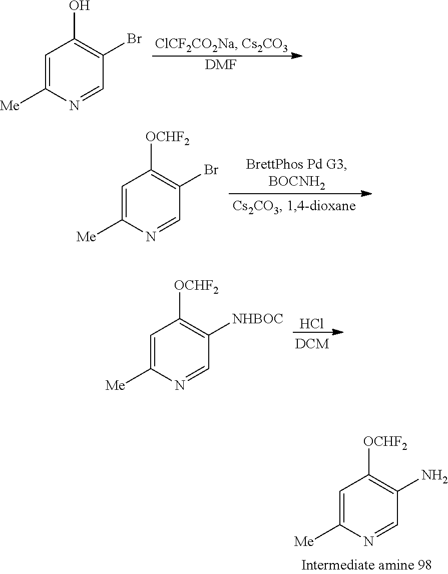

Step 1: In a dried, round-bottom flask equipped with a magnetic stirrer was suspended cesium carbonate (3 equiv, Sigma-Aldrich) in DMF (0.30 M). The reaction suspension was heated to 100° C. before a DMF solution (0.30 M) of 5-bromo-2-methylpyridin-4-ol (1 equiv, Combi-Blocks) and sodium chlorodifluoroacetate (1.5 equiv, TCI) was added dropwise over a period of 10 min. Following the completion of addition, the now orange reaction solution was heated at 100° C. for an extra 60 min before it was cooled to RT, diluted with EtOAc, and washed sequentially with water and brine. The organic layer was then dried over Na₂SO₄, filtered, and the filtrate concentrated in vacuo. Purification of the crude product thus obtained by way of column chromatography (SiO₂, gradient elution: Hex→EtOAc) afforded 5-bromo-4-(difluoromethoxy)-2-methylpyridine as a colorless oil (47% yield).

Step 2: In a thick-walled glass reaction vessel equipped with a magnetic stirrer and a Teflon screwcap was combined 5-bromo-4-(difluoromethoxy)-2-methylpyridine (1 equiv) from the previous step, cesium carbonate (3 equiv, Sigma-Aldrich), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (0.1 equiv, Sigma-Aldrich), and tert-butyl carbamate (50 equiv, Combi-Blocks) in 1,4-dioxane (0.01 M). The resulting yellow suspension was deoxygenated via subsurface purging with nitrogen for 10 min before the reaction vessel was tightly sealed and heated at 90° C. for 2 h. The reaction mixture was then cooled to RT, diluted with EtOAc, and washed sequentially with water and brine. The organic layer was then dried over Na₂SO₄, filtered, and the filtrate concentrated in vacuo. Purification of the crude product thus obtained by way of column chromatography (SiO₂, gradient elution: Hex→EtOAc) afforded tert-butyl (4-(difluoromethoxy)-6-methylpyridin-3-yl)carbamate as a white solid (87% yield).

Step 3: In a dried, round-bottom flask equipped with a magnetic stirrer was dissolved tert-butyl (4-(difluoromethoxy)-6-methylpyridin-3-yl)carbamate (1 equiv) from the previous step in dichloromethane (0.11 M). To this solution was then added HCl (4 M solution in 1,4-dioxane, 10 equiv, Sigma-Aldrich) and the resulting reaction mixture was stirred at RT for 2 h. The now white suspension was diluted with water, rendered slightly basic (pH˜8) with the addition of saturated aq. NaHCO₃, and extracted with EtOAc. The combined organic extracts were washed further with brine, dried over Na₂SO₄, filtered and the filtrate concentrated in vacuo. Purification of the crude product thus obtained by way of column chromatography (SiO₂, gradient elution: Hex→EtOAc) afforded the title compound as a dark solid (62% yield).

The following amine was prepared in an analogous fashion to Intermediate amine 98, but substituting 5-bromo-2-methylpyridin-4-ol in step 1 with the requisite, commercially available (hetero)aryl alcohol.

| Starting Material | Product |
|---|---|
| 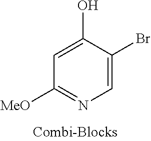 | 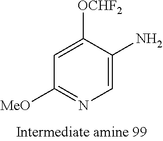 [SMILES: COc1cc(OC(F)F)c(N)cn1] |

### Intermediate amine 100: Preparation of 3-(difluoromethoxy)-5-fluoropyridin-2-amine

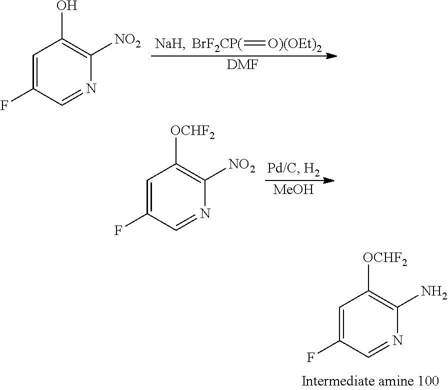

Step 1: In a dried, round-bottom flask equipped with a magnetic stirrer was suspended 5-fluoro-2-nitropyridin-3-ol (1 equiv, Combi-Blocks) in DMF (0.48 M). To this was then added at 0° C. sodium hydride (60% w/w dispersion in paraffin oil, 2.8 equiv, Sigma-Aldrich) in one rapid portion and the resulting mixture was stirred at 0° C. for 30 min to afford a brown solution. Then, diethyl (bromodifluoromethyl)phosphonate (2 equiv, Sigma-Aldrich) was added neat and dropwise over a period of 5 min, during which time a mild exotherm was observed. After completion of addition, the reaction mixture was warmed to RT and stirred at RT for another 16 h. The crude reaction mixture was then carefully quenched with water and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→7:3 (v/v) Hex: EtOAc) afforded 3-(difluoromethoxy)-5-fluoro-2-nitropyridine as a colorless oil (36% yield).

Step 2: In a dried, round-bottom flask equipped with a magnetic stirrer was dissolved 3-(difluoromethoxy)-5-fluoro-2-nitropyridine (1 equiv) from the previous step in methanol (0.35 M). The resulting solution was then deoxygenated via subsurface purging with nitrogen for 10 min before palladium (10% w/w over activated carbon, dry, 0.04 equiv, Sigma-Aldrich) was added in one rapid portion. The resulting black suspension was then subsurface purged with hydrogen for 10 min before it was stirred under a static hydrogen atmosphere (maintained with a balloon) at RT for 2 h. The reaction was subsequently diluted with EtOAc and filtered through a bed of dichloromethane-wetted celite. The insolubles were washed further with EtOAc. Concentration of the filtrate thus obtained in vacuo afforded the title compound as a yellow solid (78% yield).

### Intermediate amine 101: Preparation of 2-(difluoromethoxy)-4,5-difluoroaniline

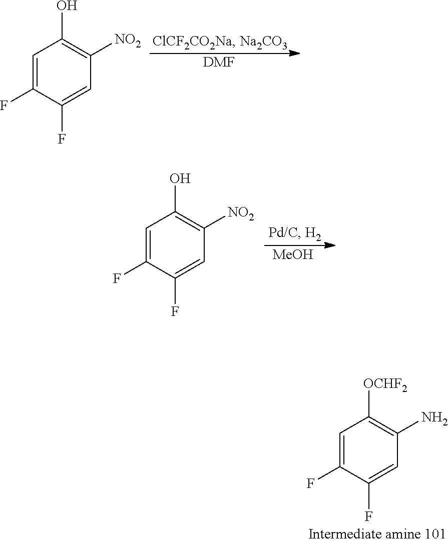

Step 1: In a dried, round-bottom flask equipped with a magnetic stirrer was combined 4,5-difluoro-2-nitrophenol (1 equiv, Combi-Blocks) and sodium carbonate (1 equiv, Sigma-Aldrich) in DMF (0.29 M). The resulting suspension was heated to 90° C. before sodium chlorodifluoroacetate (1.2 equiv, TCI) was added as a DMF solution (0.2 M), dropwise, over a period of 10 min. Following the completion of addition, the now dark reaction solution was heated at 90° C. for an extra 2 h. The reaction suspension was then cooled to RT, quenched with water, and extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: Hex→4:1 (v/v) Hex: EtOAc) afforded 1-(difluoromethoxy)-4,5-difluoro-2-nitrobenzene as a yellow oil (37% yield).

Step 2: In a dried, round-bottom flask equipped with a magnetic stirrer was dissolved 1-(difluoromethoxy)-4,5-difluoro-2-nitrobenzene (1 equiv) from the previous step in methanol (0.1 M). The resulting solution was then deoxygenated via subsurface purging with nitrogen for 10 min before palladium (10% w/w over activated carbon, dry, 0.05 equiv, Sigma-Aldrich) was added in one rapid portion. The resulting black suspension was then subsurface purged with hydrogen for 10 min before it was stirred under a static hydrogen atmosphere (maintained with a balloon) at RT for 16 h. The reaction was subsequently diluted with EtOAc and filtered through a bed of dichloromethane-wetted celite. The insolubles were washed further with EtOAc. Concentration of the filtrate thus obtained in vacuo afforded the title compound as a yellow solid (96% yield).

The following amine was prepared in an analogous fashion to Intermediate amine 101, but substituting 4,5-difluoro-2-nitrophenol in step 1 with the requisite, commercially available (hetero)aryl alcohol.

| Starting Material | Product |
|---|---|
| 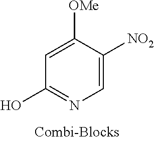 [SMILES: COc1cc(O)ncc1[N+](=O)[O-]] | 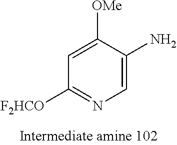 [SMILES: COc1cc(OC(F)F)ncc1N] |

### Intermediate amine 103: Preparation of (S)-6-chloro-4-((1-methoxypropan-2-yl)oxy)pyridin-3-amine

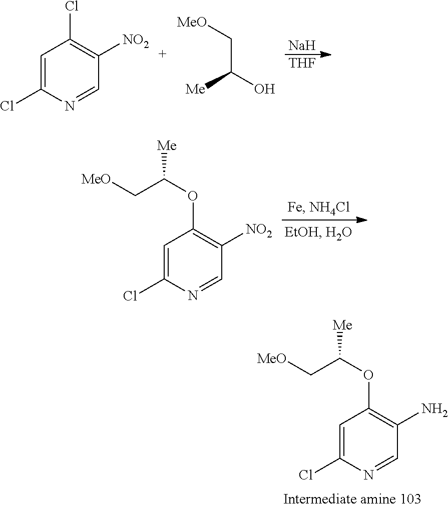

Step 1: In a dried, round-bottom flask equipped with a magnetic stirrer was diluted (S)-1-methoxypropan-2-ol (1.1 equiv, Enamine) with anhydrous THE (0.35 M). To this was then added sodium hydride (60% w/w dispersion in paraffin oil, 2 equiv, Sigma-Aldrich) in one rapid portion at 0° C. and the resulting mixture was stirred at 0° C. for 15 min. Then, 2,4-dichloro-5-nitropyridine (1 equiv, Ambeed) was added at 0° C. portionwise over a period of 5 min and the resulting mixture was allowed to warm to RT. After 2 h of stirring at RT, the crude reaction mixture was carefully quenched with ice water and then extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution, 7:1 (v/v) Hex: EtOAc→1:4 (v/v) Hex: EtOAc) afforded (S)-2-chloro-4-((1-methoxypropan-2-yl)oxy)-5-nitropyridine as a yellow oil (70% yield).

Step 2: In a dried, round-bottom flask equipped with a magnetic stirrer was combined (S)-2-chloro-4-((1-methoxypropan-2-yl)oxy)-5-nitropyridine (1 equiv) from the previous step and ammonium chloride (5 equiv, Fisher Scientific) in a 2:1 (v/v) solution of ethanol and water (0.24 M). To this was then added iron powder (5 equiv, Sigma-Aldrich) in one rapid portion and the resulting grey suspension was heated at 90° C. under a nitrogen atmosphere for 2 h. The reaction suspension was then cooled to RT, filtered through a bed of celite, and the insolubles washed with EtOAc. The filtrate was then diluted with water and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex→1:3 (v/v) Hex: EtOAc) afforded the title compound as a yellow oil (75% yield).

The following amine was prepared in an analogous fashion to Intermediate amine 103, but substituting (S)-1-methoxypropan-2-ol in step 1 with the requisite, commercially available alcohol.

| Starting Material | Product |
|---|---|
| 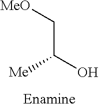 | 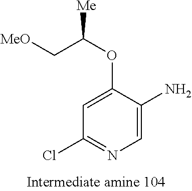 [SMILES: COC[C@@H](C)Oc1cc(Cl)ncc1N] |

### Intermediate amine 105: Preparation of 6-methyl-2-(2,2,2-trifluoroethoxy)pyridin-3-amine

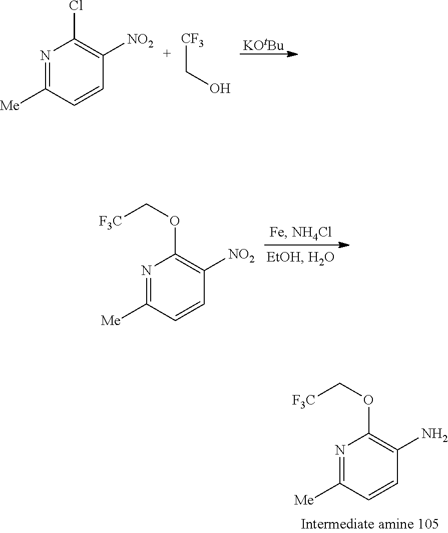 [SMILES: Cc1ccc([N+](=O)[O-])c(Cl)n1 ; OCC(F)(F)F ; Cc1ccc([N+](=O)[O-])c(OCC(F)(F)F)n1 ; Cc1ccc(N)c(OCC(F)(F)F)n1 ; "KOtBu"]

Step 1: In a dried, round-bottom flask equipped with a magnetic stirrer was dissolved 2-chloro-6-methyl-3-nitropyridine (1.1 equiv, Combi-Blocks) in 2,2,2-trifluoroethanol (0.14 M). To this was then added potassium tert-butoxide (2 equiv, Sigma-Aldrich) in one rapid portion and the resulting mixture was heated at 70° C. for 16 h. After cooling to RT, the reaction was quenched with water and extracted with EtOAc. The combined organic extracts were washed further with brine, dried over Na₂SO₄, filtered, and the filtrate concentrated in vacuo. Purification of the crude product thus obtained by way of column chromatography (SiO₂, 5:1 (v/v) Hex: EtOAc) afforded 6-methyl-3-nitro-2-(2,2,2-trifluoroethoxy)pyridine as a white solid (76% yield).

Step 2: In a dried, round-bottom flask equipped with a magnetic stirrer was combined 6-methyl-3-nitro-2-(2,2,2-trifluoroethoxy)pyridine (1 equiv) from the previous step and ammonium chloride (10 equiv, Fisher Scientific) in a 1:1 (v/v) solution of ethanol and water (0.05 M). To this was then added iron powder (10 equiv, Sigma-Aldrich) in one rapid portion and the resulting grey suspension was heated at 80° C. under a nitrogen atmosphere for 2 h. The reaction suspension was then cooled to RT, filtered through a bed of celite, and the insolubles washed with EtOAc. The filtrate was then diluted with water and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex→1:3 (v/v) Hex: EtOAc) afforded the title compound as a yellow oil (80% yield).

The following amines were prepared in an analogous fashion to Intermediate amine 105, but substituting 2,2,2-trifluoroethanol in step 1 with the requisite, commercially available alcohol. For the preparation of Intermediate amine 106, potassium tert-butoxide was also substituted with sodium ethoxide (Sigma Aldrich) in step 1. For the preparation of Intermediate amine 163 and Intermediate amine 172, potassium tert-butoxide was also substituted with cesium carbonate (Sigma Aldrich) in step 1.

| Starting Material | Product |
|---|---|
| EtOH Sigma-Aldrich | 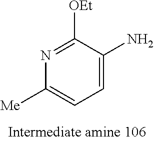 [SMILES: CCOc1nc(C)ccc1N] |
| 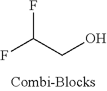 | 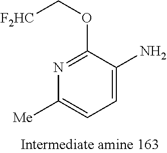 [SMILES: Cc1ccc(N)c(OCC(F)F)n1] |
| 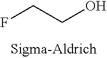 | 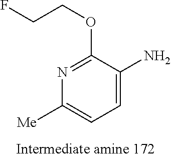 [SMILES: Cc1ccc(N)c(OCCF)n1] |

### Intermediate amine 107: Preparation of 2-(2,2-difluoroethoxy)-6-methoxypyridin-3-amine

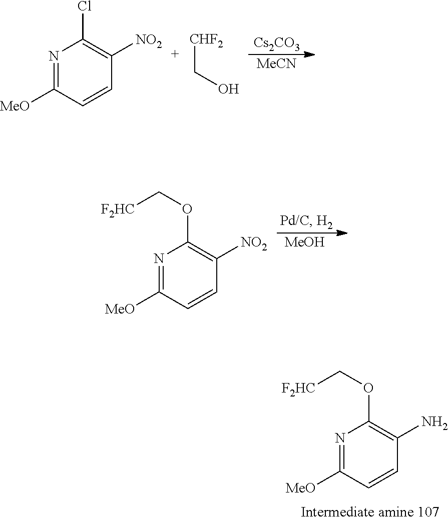

Step 1: In a dried, round-bottom flask equipped with a magnetic stirrer was combined 2-chloro-6-methoxy-3-nitropyridine (1 equiv, Combi-Blocks) and 2,2-difluoroethan-1-ol (3 equiv, Combi-Blocks) in acetonitrile (0.18 M). To this was then added cesium carbonate (3 equiv, Sigma-Aldrich) in one rapid portion and the resulting mixture was heated at 80° C. for 2 h. After cooling to RT, the reaction was quenched with water and extracted with EtOAc. The combined organic extracts were washed further with brine, dried over Na₂SO₄, filtered, and the filtrate concentrated in vacuo. Purification of the crude product thus obtained by way of column chromatography (SiO₂, 5:1 (v/v) Hex: EtOAc) afforded 2-(2,2-difluoroethoxy)-6-methoxy-3-nitropyridine as a yellow oil (97% yield).

Step 2: In a dried, round-bottom flask equipped with a magnetic stirrer was dissolved 2-(2,2-difluoroethoxy)-6-methoxy-3-nitropyridine (1 equiv) from the previous step in methanol (0.13 M). The resulting solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 1 h. The reaction was subsequently quenched with dichloromethane and filtered through a bed of dichloromethane-wetted celite. Concentration of the filtrate concentrated in vacuo afforded the title compound as a brown oil (92% yield).

The following amines were prepared in an analogous fashion to Intermediate amine 107, but substituting 2,2-difluoroethan-1-ol in step 1 with the requisite, commercially available alcohol. For the preparation of Intermediate amine 164 and Intermediate amine 173, cesium carbonate and acetonitrile were also substituted with sodium hydride (60% w/w dispersion in paraffin oil, Sigma Aldrich) and THF, respectively, in step 1.

| Starting Material | Product |
|---|---|
| 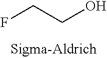 | 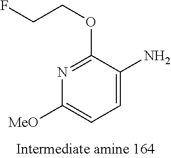 [SMILES: COc1ccc(N)c(OCCF)n1] |
| 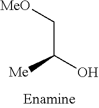 | 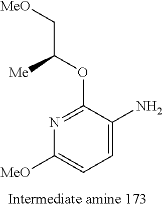 [SMILES: COC[C@H](C)Oc1nc(OC)ccc1N] |

### Intermediate amine 108: Preparation of 2-(difluoromethoxy)-6-ethoxypyridin-3-amine

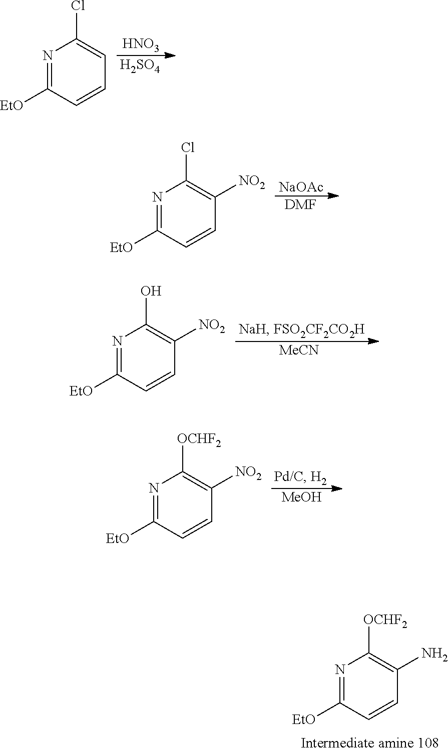

Step 1: In a dried, round-bottom flask equipped with a magnetic stirrer was added 2-chloro-6-ethoxypyridine (1 equiv, Combi-Blocks), neat and dropwise, to a 2:1 (v/v) solution of concentrated sulfuric acid and fuming nitric acid (0.53 M). The resulting yellow solution was first stirred at RT for 10 min, and then heated at 50° C. for 50 min. The reaction was then carefully quenched with ice water and extracted with EtOAc. The combined organic extracts were washed further with water and brine, dried over MgSO₄, and filtered. Concentration of the filtrate thus obtained in vacuo afforded crude 2-chloro-6-ethoxy-3-nitropyridine as a yellow solid (84% yield).

Step 2: In a dried, round-bottom flask equipped with a magnetic stirrer was combined 2-chloro-6-ethoxy-3-nitropyridine (1 equiv) from the previous step and sodium acetate (3 equiv, Sigma-Aldrich) in DMF (0.53 M). The resulting solution was then heated at 100° C. for 6 h. After cooling to RT, the reaction was quenched with water and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→2:3 (v/v) Hex: EtOAc) afforded 6-ethoxy-3-nitropyridin-2-ol as a yellow solid (38% yield).

Step 3: In a dried, round-bottom flask equipped with a magnetic stirrer was dissolved 6-ethoxy-3-nitropyridin-2-ol (1 equiv) from the previous step in acetonitrile (0.073 M). To this was then added sodium hydride (60% w/w dispersion in paraffin oil, 3.2 equiv, Sigma-Aldrich) in one rapid portion and the resulting mixture was stirred at RT for 20 min to afford a brown suspension. Then, 2,2-difluoro-2-(fluorosulfonyl)acetic acid (2 equiv, Sigma-Aldrich) was added neat and dropwise over a period of 2 min, during which time a mild exotherm was observed. After 1 h of stirring at RT, the crude reaction mixture was carefully quenched with water and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 2-(difluoromethoxy)-6-ethoxy-3-nitropyridine as a yellow solid (33% yield).

Step 4: In a dried, round-bottom flask equipped with a magnetic stirrer was dissolved 2-(difluoromethoxy)-6-ethoxy-3-nitropyridine (1 equiv) from the previous step in a 1:1 (v/v) solution of EtOAc and methanol (0.12 M). The resulting yellow solution was then deoxygenated via subsurface purging with nitrogen for 10 min before palladium (10% w/w over activated carbon, dry, 0.1 equiv, Sigma-Aldrich) was added in one rapid portion. The resulting black suspension was then subsurface purged with hydrogen for 10 min before it was stirred under a static hydrogen atmosphere at RT for 45 min. The reaction was subsequently diluted with EtOAc, filtered through a bed of dichloromethane-wetted celite, and the filtrate concentrated in vacuo. Purification of the crude product thus obtained by way of column chromatography (SiO₂, gradient elution: Hex→1:1 (v/v) Hex: EtOAc) afforded the title compound as a white, crystalline solid (54% yield).

### Intermediate amine 109 & Intermediate amine 110: Preparation of 2-chloro-6-(difluoromethoxy)pyridin-3-amine and 6-chloro-2-(difluoromethoxy)pyridin-3-amine

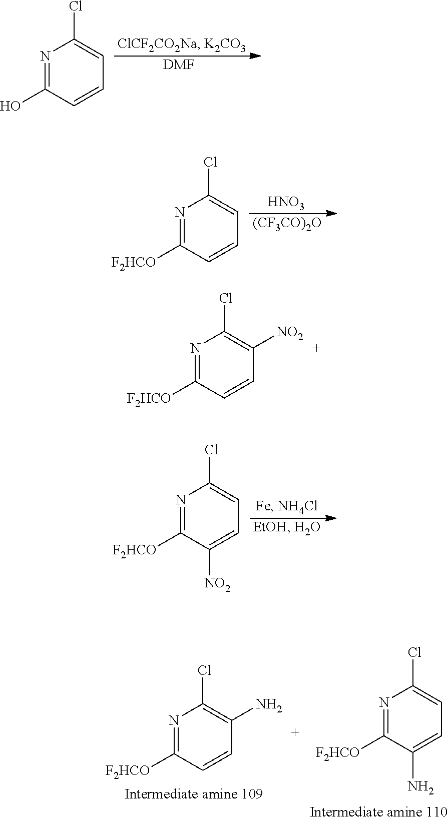

Step 1: In a dried, round-bottom flask equipped with a magnetic stirrer was suspended 6-chloropyridin-2-ol (1 equiv, Combi-Blocks) and potassium carbonate (1 equiv, Sigma-Aldrich) in DMF (0.10 M). The resulting suspension was heated to 85° C. before sodium chlorodifluoroacetate (1.2 equiv, TCI) was added as a DMF solution (0.2 M), dropwise, over a period of 10 min. Following the completion of addition, the now dark reaction mixture was heated at 85° C. for an extra 50 min and then at 100° C. for 2 h. The reaction suspension was then cooled RT, diluted with EtOAc, and washed sequentially with water and brine. The organic layer was then dried over MgSO₄ and filtered. Concentration of the filtrate thus obtained in vacuo afforded crude 2-chloro-6-(difluoromethoxy)pyridine as a white solid (14% yield).

Step 2: In a dried, round-bottom flask equipped with a magnetic stirrer was dissolved 2-chloro-6-(difluoromethoxy)pyridine (1 equiv) from the previous step in trifluoroacetic anhydride (1.8 M). To this mixture was then added, at 0° C., fuming nitric acid (1.1 equiv, Fisher Scientific) over a period of 5 min. The resulting yellow reaction solution was stirred at 0° C. for 2 h, before it was carefully quenched with ice water and extracted with tert-butyl methyl ether. The combined organic extracts were washed further with water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 2-chloro-6-(difluoromethoxy)-3-nitropyridine and 6-chloro-2-(difluoromethoxy)-3-nitropyridine as an inseparable mixture (90% combined yield).

Step 3: In a dried, round-bottom flask equipped with a magnetic stirrer was combined the inseparable mixture of 2-chloro-6-(difluoromethoxy)-3-nitropyridine and 6-chloro-2-(difluoromethoxy)-3-nitropyridine (1 equiv) from the previous step, and ammonium chloride (5 equiv, Fisher Scientific) in a 2:1 (v/v) solution of ethanol and water (0.67 M). To this solution was then added iron powder (5 equiv, Sigma-Aldrich) in one rapid portion and the resulting grey suspension was heated at 80° C. under a nitrogen atmosphere for 2 h. The reaction suspension was then cooled to RT, filtered through a bed of celite, and the insolubles washed with EtOAc. The filtrate thus obtained was then diluted with water and extracted further with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded the two title compounds (91% combined yield).

### Intermediate amine 111: Preparation of 5-fluoro-2,6-dimethoxypyridin-3-amine

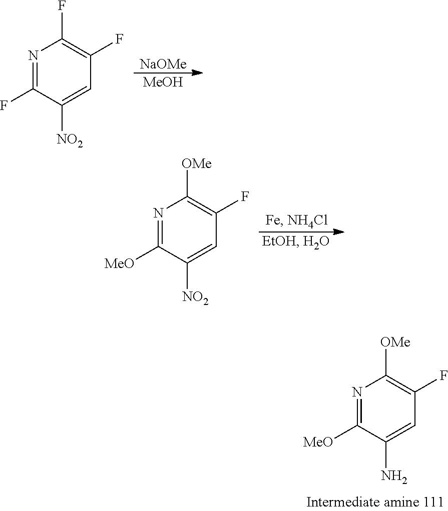

Step 1: In a dried, round-bottom flask equipped with a magnetic stirrer was dissolved 2,3,6-trifluoro-5-nitropyridine (1 equiv, Ambeed) in anhydrous methanol (0.28 M). To this mixture was then added at −40° C. sodium methoxide (4 equiv, Sigma-Aldrich) in one rapid portion and the resulting mixture was stirred at −40° C. for 2 h. The reaction was then carefully quenched with HCl (1 M solution in water, 4 equiv) and extracted with EtOAc. The combined organic extracts were washed further with brine, dried over Na₂SO₄, and filtered. Concentration of the filtrate in vacuo afforded crude 3-fluoro-2,6-dimethoxy-5-nitropyridine that was used immediately without further purification.

Step 2: In a dried, round-bottom flask equipped with a magnetic stirrer was combined 3-fluoro-2,6-dimethoxy-5-nitropyridine (1 equiv) from the previous step and ammonium chloride (10 equiv, Fisher Scientific) in a 1:1 (v/v) solution of ethanol and water (0.15 M). To this solution was then added iron powder (10 equiv, Sigma-Aldrich) in one rapid portion and the resulting grey suspension was heated at 100° C. under a nitrogen atmosphere for 1 h. The reaction suspension was then cooled to RT, filtered through a bed of celite, and the insolubles washed with DCM. The filtrate thus obtained was then diluted with water and extracted further with DCM. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded the title compound as a brown solid (62% yield over two steps).

### Intermediate amine 112: Preparation of 2,4-dimethoxy-3-methylaniline

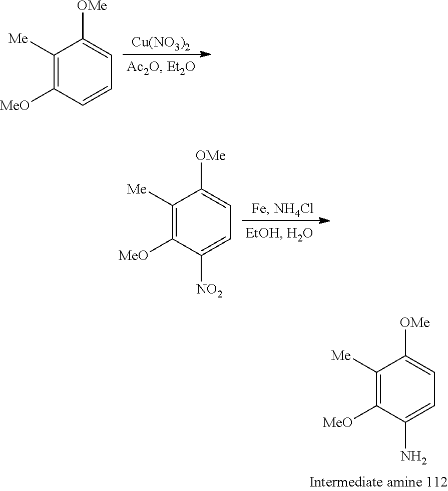

Step 1: In a dried, round-bottom flask equipped with a magnetic stirrer was combined 1,3-dimethoxy-2-methylbenzene (1 equiv, Combi-Blocks) and acetic anhydride (10 equiv, Sigma-Aldrich) in diethyl ether (0.21 M). To this mixture was then added cupric nitrate (2.5 equiv, Sigma-Aldrich) in one rapid portion and the resulting blue reaction solution was stirred at RT for 30 min. The reaction was quenched with water and extracted with EtOAc. The combined organic extracts were washed further with brine, dried over Na₂SO₄, and filtered. Concentration of the filtrate in vacuo afforded crude 1,3-dimethoxy-2-methyl-4-nitrobenzene that was used immediately without further purification.

Step 2: In a dried, round-bottom flask equipped with a magnetic stirrer was combined 1,3-dimethoxy-2-methyl-4-nitrobenzene (1 equiv) from the previous step, and ammonium chloride (10 equiv, Fisher Scientific) in a 1:1 (v/v) solution of ethanol and water (0.15 M). To this solution was then added iron powder (10 equiv, Sigma-Aldrich) in one rapid portion and the resulting grey suspension was heated at 80° C. under a nitrogen atmosphere for 2 h. The reaction suspension was then cooled to RT, filtered through a bed of celite, and the insolubles washed with DCM. The filtrate thus obtained was then diluted with water and extracted further with DCM. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded the title compound as a black solid (23% yield over two steps).

### Intermediate amine 113: Preparation of 5-chloro-3-(difluoromethoxy)pyridin-2-amine

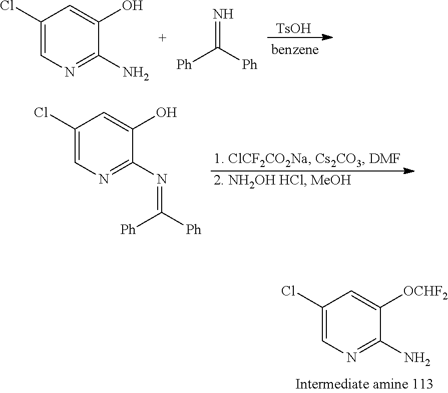 [SMILES: Nc1ncc(Cl)cc1O ; N=C(c1ccccc1)c1ccccc1 ; "TsOH" ; Oc1cc(Cl)cnc1N=C(c1ccccc1)c1ccccc1 ; "2. NH2OH HCl, MeOH" ; Nc1ncc(Cl)cc1OC(F)F]

Step 1: In a dried, round-bottom flask equipped with a magnetic stirrer was suspended 2-amino-5-chloropyridin-3-ol (1 equiv, Combi-Blocks), benzophenone imine (1 equiv, Combi-Blocks), and p-toluenesulfonic acid (a few crystals, Combi-Blocks) in benzene (0.1 M). A Dean-Stark trap was attached, and the resulting mixture was heated to reflux for 16 h. The now yellow suspension was cooled to RT, diluted with dichloromethane, and washed with water. The organic extract thus obtained was then dried over MgSO₄, filtered, and the filtrate concentrated in vacuo. The crude product thus obtained was then triturated in hexanes to afford 5-chloro-2-((diphenylmethylene)amino)pyridin-3-ol as a yellow solid (40% yield).

Step 2: In a dried, round-bottom flask equipped with a magnetic stirrer was suspended 5-chloro-2-((diphenylmethylene)amino)pyridin-3-ol (1 equiv) from the previous step and cesium carbonate (1.1 equiv, Sigma-Aldrich) in DMF (0.19 M). The resulting suspension was heated to 90° C. before sodium chlorodifluoroacetate (1.1 equiv, TCI) was added as a DMF solution (0.2 M), dropwise over a period of 30 min. Following the completion of addition, the now dark reaction solution was heated at 90° C. for an extra 30 min before it was cooled to RT, diluted with tert-butyl methyl ether, and washed sequentially with 1 M aq. NaOH, water and brine. The organic layer was then dried over MgSO₄, filtered, and the filtrate concentrated in vacuo. The resulting residue was then taken up in methanol (0.06 M) and added hydroxylamine hydrochloride (2 equiv, Sigma-Aldrich). After 3 h of stirring at RT, the volatiles were then removed in vacuo and the resulting residue partitioned between EtOAc and water. The organic layer was separated, washed further with water and brine, dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded the title compound as an off-white solid (32% yield).

### Intermediate amine 114: Preparation of 2-(difluoromethoxy)-6-(methyl-d₃)pyridin-3-amine

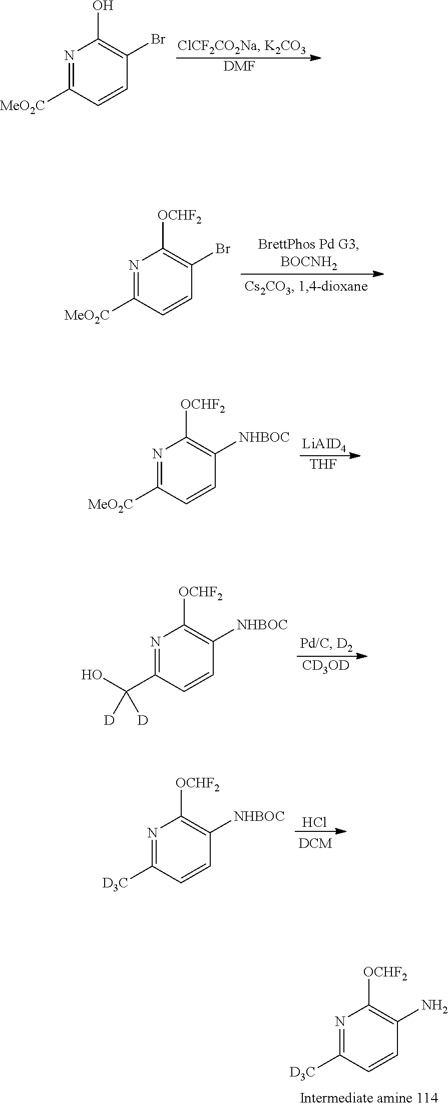

Step 1: In a dried, round-bottom flask equipped with a magnetic stirrer was suspended potassium carbonate (4 equiv, Sigma-Aldrich) in DMF (0.69 M). The reaction suspension was heated to 100° C. before a DMF solution (0.43 M) of methyl 5-bromo-6-hydroxypicolinate (1 equiv, Combi-Blocks) and sodium chlorodifluoroacetate (2 equiv, TCI) was added dropwise over a period of 10 min. Following the completion of addition, the now orange reaction solution was heated at 100° C. for an extra 45 min before it was cooled to RT, diluted with tert-butyl methyl ether, and washed sequentially with water and brine. The organic layer was then dried over MgSO₄, filtered, and the filtrate concentrated in vacuo. Purification of the crude product thus obtained by way of column chromatography (SiO₂, gradient elution: 9:1 (v/v) Hex: EtOAc→1:1 (v/v) Hex: EtOAc) afforded methyl 5-bromo-6-(difluoromethoxy)picolinate as a white solid (83% yield).

Step 2: In a thick-walled glass reaction vessel equipped with a magnetic stirrer and a Teflon screwcap was combined methyl 5-bromo-6-(difluoromethoxy)picolinate (1 equiv) from the previous step, cesium carbonate (3 equiv, Sigma-Aldrich), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (0.1 equiv, Sigma-Aldrich), and tert-butyl carbamate (3 equiv, Combi-Blocks) in 1,4-dioxane (0.06 M). The resulting yellow suspension was deoxygenated via subsurface purging with nitrogen for 10 min before the reaction vessel was tightly sealed and heated at 100° C. for 48 h. The reaction mixture was then cooled to RT, diluted with EtOAc, and washed sequentially with water and brine. The organic layer was then dried over MgSO₄, filtered, and the filtrate concentrated in vacuo. Purification of the crude product thus obtained by way of column chromatography (SiO₂, gradient elution: 9:1 (v/v) Hex: EtOAc→1:1 (v/v) Hex: EtOAc) afforded methyl 5-((tert-butoxycarbonyl)amino)-6-(difluoromethoxy)picolinate as a white solid (35% yield).

Step 3: In a dried, round-bottom flask equipped with a magnetic stirrer was dissolved methyl 5-((tert-butoxycarbonyl)amino)-6-(difluoromethoxy)picolinate (1 equiv) from the previous step in THF (0.084 M). To this solution was then added at 0° C. lithium aluminum deuteride (3 equiv, Sigma-Aldrich) in one rapid portion, and the resulting suspension was stirred first at 0° C. for 30 min and then at RT for 18 h. The reaction was then carefully quenched with the sequential addition of water and NaOH (1 M solution in water, 10 equiv). The resulting suspension was vigorously stirred at RT for 30 min, and then extracted with EtOAc. The combined organic extracts were then dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded tert-butyl (2-(difluoromethoxy)-6-(hydroxymethyl-d₂)pyridin-3-yl)carbamate as a colorless oil (71% yield).

Step 4: In a dried, round-bottom flask equipped with a magnetic stirrer was dissolved tert-butyl (2-(difluoromethoxy)-6-(hydroxymethyl-d₂)pyridin-3-yl)carbamate (1 equiv) from the previous step in methanol-d₄ (0.15 M). The resulting colorless solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with deuterium gas for 10 min before it was stirred under a static deuterium atmosphere (maintained with a balloon) at RT for 16 h. The reaction was subsequently quenched with dichloromethane and filtered through a bed of dichloromethane-wetted celite. Concentration of the filtrate thus obtained in vacuo furnished crude tert-butyl (2-(difluoromethoxy)-6-(methyl-d₃)pyridin-3-yl)carbamate as a colorless oil (90% yield).

Step 5: In a dried, round-bottom flask equipped with a magnetic stirrer was dissolved tert-butyl (2-(difluoromethoxy)-6-(methyl-d₃)pyridin-3-yl)carbamate (1 equiv) from the previous step in dichloromethane (0.068 M). To this solution was then added HCl (4 M solution in 1,4-dioxane, 4 equiv, Sigma-Aldrich) and the resulting reaction mixture was stirred at RT for 16 h. The now white suspension was then diluted with a 1:1 (v/v) solution of hexanes and tert-butyl methyl ether, sonicated for 10 min, and filtered. The insolubles were then re-taken up in DCM and washed further with NaOH (1 M solution in water, 10 equiv). The organic layer was then separated, dried over MgSO₄, and filtered. Concentration of the filtrate thus obtained in vacuo furnished the title compound as a colorless oil (71% yield).

### Intermediate amine 115: Preparation of 3-(difluoromethoxy)-5-methoxypyridin-2-amine

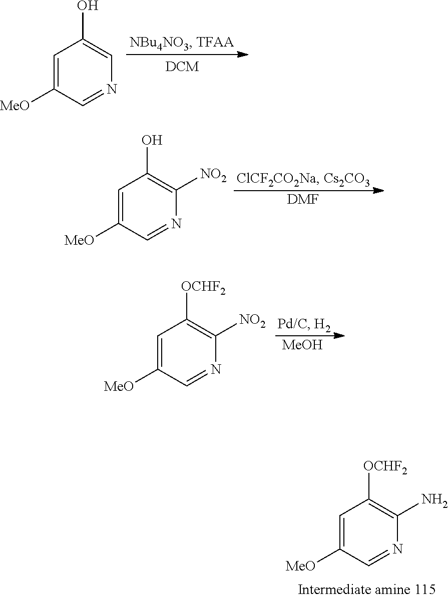

Step 1: In a dried, round-bottom flask equipped with a magnetic stirrer was combined 5-methoxypyridin-3-ol (1 equiv, Combi-Blocks) and tetrabutylammonium nitrate (1.6 equiv, Sigma-Aldrich) in dichloromethane (0.16 M). The resulting solution was then cooled to 0° C. before trifluoroacetic anhydride (1.6 equiv, Sigma-Aldrich) was added neat and dropwise. The resulting mixture was stirred at 0° C. for 2 h and then at RT for an additional 30 min. The reaction was then quenched with water and extracted further with dichloromethane. The combined organic extracts were washed further with water and brine, dried over Na₂SO₄, filtered, and the filtrate concentrated in vacuo. Purification of the crude product thus obtained by way of column chromatography (SiO₂, 3:1 (v/v) Hex: EtOAc) afforded 5-methoxy-2-nitropyridin-3-ol as a yellow oil (31% yield).

Step 2: In a dried, round-bottom flask equipped with a magnetic stirrer was suspended cesium carbonate (3 equiv, Sigma-Aldrich) in DMF (0.42 M). The reaction suspension was heated to 80° C. before a DMF solution (0.28 M) of 5-methoxy-2-nitropyridin-3-ol (1 equiv) from the previous step and sodium chlorodifluoroacetate (1.5 equiv, TCI) was added dropwise over a period of 10 min. Following the completion of addition, the now orange reaction solution was heated at 80° C. for an extra 30 min before it was cooled to RT, diluted with EtOAc, and washed sequentially with water and brine. The organic layer was then dried over Na₂SO₄, filtered, and the filtrate concentrated in vacuo. Purification of the crude product thus obtained by way of column chromatography (SiO₂, 7:1 (v/v) Hex: EtOAc) afforded 3-(difluoromethoxy)-5-methoxy-2-nitropyridine as a yellow oil (32% yield).

Step 3: In a dried, round-bottom flask equipped with a magnetic stirrer was dissolved 3-(difluoromethoxy)-5-methoxy-2-nitropyridine (1 equiv) from the previous step in methanol (0.023 M). The resulting yellow solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 1 h. The reaction was subsequently quenched with dichloromethane and filtered through a bed of dichloromethane-wetted celite. Concentration of the filtrate in vacuo afforded the title compound as a yellow solid (97% yield).

### Intermediate amine 116: Preparation of 6-(difluoromethyl)-2-methoxypyridin-3-amine

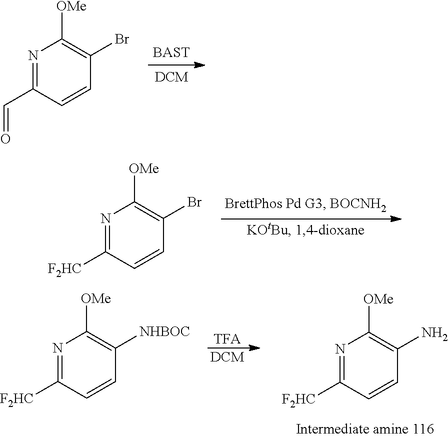

Step 1: In a dried, round-bottom flask equipped with a magnetic stirrer was dissolved 5-bromo-6-methoxypicolinaldehyde (1 equiv, Ambeed) in dichloromethane (0.12 M). To this solution was then added at 0° C. bis(2-methoxyethyl)aminosulfur trifluoride (2.5 equiv, Sigma-Aldrich), neat and dropwise, over a period of 10 min. Following the completion of addition, the now orange reaction solution was warmed to and stirred at RT for 16 h. The reaction was then carefully quenched with ice water and extracted with DCM. The combined organic extracts were then washed sequentially with saturated aq. NaHCO₃, water and brine. The organic layer was then dried over Na₂SO₄, filtered, and the filtrate concentrated in vacuo. Purification of the crude product thus obtained by way of column chromatography (SiO₂, gradient elution: Hex→EtOAc) afforded 3-bromo-6-(difluoromethyl)-2-methoxypyridine as a colorless oil (85% yield).

Step 2: In a thick-walled glass reaction vessel equipped with a magnetic stirrer and a Teflon screwcap was combined 3-bromo-6-(difluoromethyl)-2-methoxypyridine (1 equiv) from the previous step, potassium tert-butoxide (3 equiv, Sigma-Aldrich), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (0.2 equiv, Sigma-Aldrich), and tert-butyl carbamate (50 equiv, Combi-Blocks) in 1,4-dioxane (0.042 M). The resulting mixture was deoxygenated via subsurface purging with nitrogen for 10 min before the reaction vessel was tightly sealed and heated at 90° C. for 2 h. The reaction mixture was then cooled to RT, diluted with EtOAc, and washed sequentially with water and brine. The organic layer was then dried over Na₂SO₄, filtered, and the filtrate concentrated in vacuo. Purification of the crude product thus obtained by way of column chromatography (SiO₂, gradient elution: Hex→EtOAc) afforded tert-butyl (6-(difluoromethyl)-2-methoxypyridin-3-yl)carbamate as a yellow oil (87% yield).

Step 3: In a dried, round-bottom flask equipped with a magnetic stirrer was dissolved tert-butyl (6-(difluoromethyl)-2-methoxypyridin-3-yl)carbamate (1 equiv) from the previous step in dichloromethane (0.12 M). To this solution was then added trifluoroacetic acid (150 equiv, Sigma-Aldrich) and the resulting reaction mixture was stirred at RT for 1 h. The mixture was then rendered slightly basic (pH˜8) with the addition of ammonia (7 M solution in MeOH, Sigma-Aldrich) and the volatiles were removed in vacuo. Purification of the crude product thus obtained by way of reverse-phase column chromatography (C₁₈, gradient elution: 9:1 (v/v) H₂O: MeCN→MeCN) afforded the title compound as a yellow oil (91% yield).

The following amine was prepared in an analogous fashion to Intermediate amine 116, but substituting 5-bromo-6-methoxypicolinaldehyde in step 1 with the requisite, commercially available aldehyde.

| Starting Material | Product |
|---|---|
| 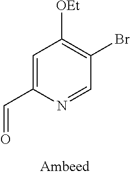 [SMILES: CCOc1cc(C=O)ncc1Br] | 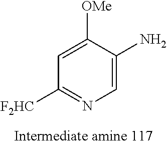 [SMILES: COc1cc(C(F)F)ncc1N] |

### Intermediate amine 118: Preparation of 2-(difluoromethoxy)-6-(difluoromethyl)pyridin-3-amine

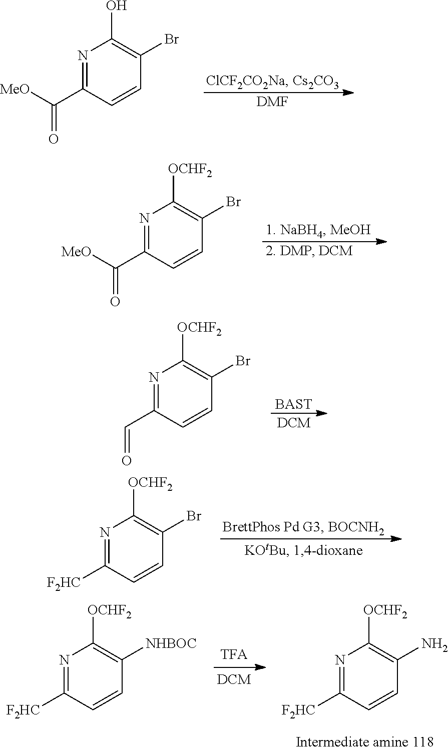

Step 1: In a dried, round-bottom flask equipped with a magnetic stirrer was suspended cesium carbonate (3 equiv, Sigma-Aldrich) in DMF (0.43 M). The reaction suspension was heated to 80° C. before a DMF solution (0.44 M) of methyl 5-bromo-6-hydroxypicolinate (1 equiv, Toronto Research Chemicals) and sodium chlorodifluoroacetate (1.2 equiv, TCI) was added dropwise over a period of 10 min. Following the completion of addition, the now orange reaction solution was heated at 80° C. for an extra 50 min before it was cooled to RT, diluted with EtOAc, and washed sequentially with water and brine. The organic layer was then dried over Na₂SO₄, filtered, and the filtrate concentrated in vacuo. Purification of the crude product thus obtained by way of column chromatography (SiO₂, 5:1 (v/v) Hex: EtOAc) afforded methyl 5-bromo-6-(difluoromethoxy)picolinate as a yellow oil (45% yield).

Step 2: In a dried, round-bottom flask equipped with a magnetic stirrer was dissolved methyl 5-bromo-6-(difluoromethoxy)picolinate (1 equiv) from the previous step in methanol (0.095 M). To this solution was then added sodium borohydride (25 equiv, Sigma-Aldrich) portionwise over 10 min, and following a period of vigorous gas evolution, the resulting mixture was stirred at RT for 2 h. The reaction was then carefully quenched with the addition of ice water and extracted with EtOAc. The combined organic extracts were then washed further with brine, dried over MgSO₄, filtered, and the filtrate concentrated in vacuo. The crude (5-bromo-6-(difluoromethoxy)pyridin-2-yl)methanol thus obtained was immediately taken up in dichloromethane (0.2 M) and added Dess-Martin periodinane (1.5 equiv, Sigma-Aldrich) in one rapid portion at RT. After 1 h of stirring at RT, the reaction was then carefully quenched with the addition of 1 M aq. NaOH and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded 5-bromo-6-(difluoromethoxy)picolinaldehyde as an off-white solid (42% yield).

Step 3: In a dried, round-bottom flask equipped with a magnetic stirrer was dissolved 5-bromo-6-(difluoromethoxy)picolinaldehyde (1 equiv) from the previous step in dichloromethane (0.12 M). To this solution was then added at 0° C. bis(2-methoxyethyl)aminosulfur trifluoride (1.5 equiv, Sigma-Aldrich), neat and dropwise, over a period of 10 min. Following the completion of addition, the now orange reaction solution was warmed to and stirred at RT for 1 h. The reaction was then carefully quenched with saturated aq. NaHCO₃ and extracted with EtOAc. The combined organic extracts were then washed further with water and brine, dried over Na₂SO₄, filtered, and the filtrate concentrated in vacuo. Purification of the crude product thus obtained by way of column chromatography (SiO₂, gradient elution: Hex→EtOAc) afforded 3-bromo-2-(difluoromethoxy)-6-(difluoromethyl)pyridine as a white solid (61% yield).

Step 4: In a thick-walled glass reaction vessel equipped with a magnetic stirrer and a Teflon screwcap was combined 3-bromo-2-(difluoromethoxy)-6-(difluoromethyl)pyridine (1 equiv) from the previous step, potassium tert-butoxide (3 equiv, Sigma-Aldrich), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (0.2 equiv, Sigma-Aldrich), and tert-butyl carbamate (10 equiv, Combi-Blocks) in 1,4-dioxane (0.029 M). The resulting mixture was deoxygenated via subsurface purging with nitrogen for 10 min before the reaction vessel was tightly sealed and heated at 90° C. for 1 h. The reaction mixture was then cooled to RT, diluted with EtOAc, and washed sequentially with water and brine. The organic layer was then dried over Na₂SO₄, filtered, and the filtrate concentrated in vacuo. Purification of the crude product thus obtained by way of column chromatography (SiO₂, gradient elution: Hex→EtOAc) afforded tert-butyl (2-(difluoromethoxy)-6-(difluoromethyl)pyridin-3-yl)carbamate as a white solid (55% yield).

Step 5: In a dried, round-bottom flask equipped with a magnetic stirrer was dissolved tert-butyl (2-(difluoromethoxy)-6-(difluoromethyl)pyridin-3-yl)carbamate (1 equiv) from the previous step in dichloromethane (0.16 M). To this solution was then added trifluoroacetic acid (150 equiv, Sigma-Aldrich) and the resulting reaction mixture was stirred at RT for 1 h. The mixture was then rendered slightly basic (pH˜8) with the addition of ammonia (7 M solution in MeOH, Sigma-Aldrich) and the volatiles were removed in vacuo. Purification of the crude product thus obtained by way of reverse-phase column chromatography (C₁₈, gradient elution: 9:1 (v/v) H₂O: MeCN→MeCN) afforded the title compound as a light brown oil (88% yield).

### Intermediate amine 119: Preparation of 4-methoxy-6,7-dihydro-5H-cyclopenta[b]pyridin-3-amine

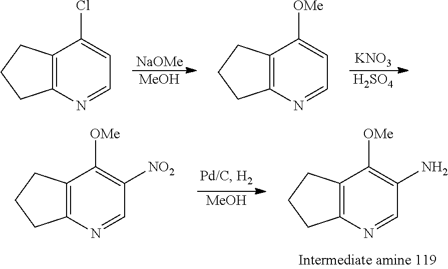

Step 1: In a thick-walled glass reaction vessel equipped with a magnetic stirrer and a Teflon screwcap was combined 4-chloro-6,7-dihydro-5H-cyclopenta[b]pyridine (1 equiv, Combi-Blocks) and sodium methoxide (5 equiv, Sigma-Aldrich) in anhydrous methanol (0.32 M). The reaction vessel was tightly sealed and heated at 110° C. for 2 days. The reaction was then cooled to RT and the volatiles were removed in vacuo. The resulting residue was then partitioned between water and EtOAc. The aqueous layer was separated and back extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: Hex→EtOAc) afforded 4-methoxy-6,7-dihydro-5H-cyclopenta[b]pyridine as a colorless oil (85% yield).

Step 2: In a dried, round-bottom flask equipped with a magnetic stirrer was added at 0° C. potassium nitrate (3 equiv, Sigma-Aldrich) portionwise to a concentrated sulfuric acid solution (0.2 M) of 4-methoxy-6,7-dihydro-5H-cyclopenta[b]pyridine (1 equiv) from the previous step. After completion of addition, the resulting solution was first stirred at RT for 10 min, and then heated at 80° C. for 16 h. The reaction was then carefully neutralized at 0° C. with the dropwise addition of 2 N aq. NaOH until a pH of ˜8 was reached, and the resulting suspension was extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded 4-methoxy-3-nitro-6,7-dihydro-5H-cyclopenta[b]pyridine as a yellow oil (51% yield).

Step 3: In a dried, round-bottom flask equipped with a magnetic stirrer was dissolved 4-methoxy-3-nitro-6,7-dihydro-5H-cyclopenta[b]pyridine (1 equiv) from the previous step in methanol (0.10 M). The resulting solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 30 min. The reaction was subsequently quenched with dichloromethane and filtered through a bed of dichloromethane-wetted celite. Concentration of the filtrate thus obtained in vacuo afforded the title compound as a yellow oil (89% yield).

### Intermediate amine 120: Preparation of 6-bromo-2-(difluoromethoxy)-5-fluoropyridin-3-amine

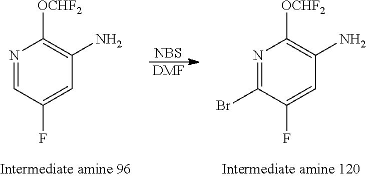

In a dried, round-bottom flask equipped with a magnetic stirrer was dissolved Intermediate amine 96 (1 equiv) in DMF (0.84 M). The resulting solution was then cooled to 0° C. before freshly recrystallized N-bromosuccinimide (1 equiv, Sigma-Aldrich) was added in one rapid portion. The resulting mixture was stirred at 0° C. for 10 min and then at RT for an additional 30 min. The reaction was then quenched with water and extracted further with tert-butyl methyl ether. The combined organic extracts were washed further with water, saturated aq. NaHCO₃ and brine, dried over Na₂SO₄, filtered, and the filtrate concentrated in vacuo. Purification of the crude product thus obtained by way of column chromatography (SiO₂, gradient elution: Hex→4:1 (v/v) Hex: EtOAc) afforded the title compound as an orange solid (74% yield).

The following amine was prepared in an analogous fashion to Intermediate amine 120, but substituting Intermediate amine 96 with the requisite, commercially available aniline.

| Starting Material | Product |
|---|---|
| 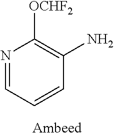 [SMILES: Nc1cccnc1OC(F)F] | 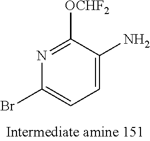 [SMILES: Nc1ccc(Br)nc1OC(F)F] |

### Intermediate amine 136: Preparation of 2,6-diisopropoxypyridin-3-amine

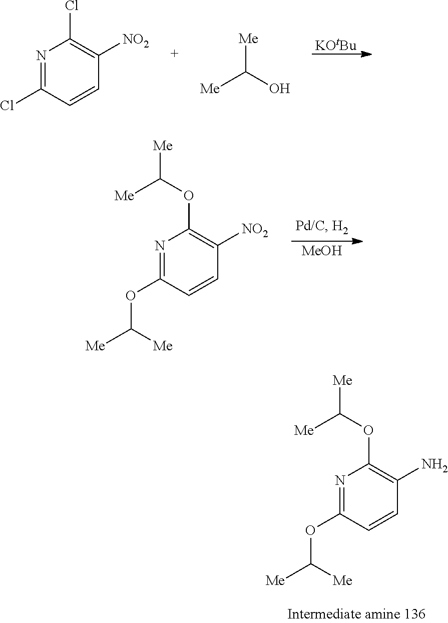

Step 1: In a dried, round-bottom flask equipped with a magnetic stirrer was dissolved 2,6-dichloro-3-nitropyridine (1 equiv, Combi-Blocks) in isopropanol (0.20 M). To this was then added potassium tert-butoxide (4 equiv, Sigma-Aldrich) in one rapid portion and the resulting mixture was stirred at RT for 1 h. The reaction was then quenched with water and extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: Hex→3:1 (v/v) Hex: EtOAc) afforded 2,6-diisopropoxy-3-nitropyridine as a yellow solid (46% yield).

Step 2: In a dried, round-bottom flask equipped with a magnetic stirrer was dissolved 2,6-diisopropoxy-3-nitropyridine (1 equiv) from the previous step in methanol (0.11 M). The resulting yellow solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 16 h. The reaction was subsequently quenched with dichloromethane and filtered through a bed of dichloromethane-wetted celite. Concentration of the filtrate in vacuo afforded the title compound as a brown solid (97% yield).

### Intermediate amine 137: Preparation of 2-(benzyloxy)-6-methoxypyridin-3-amine

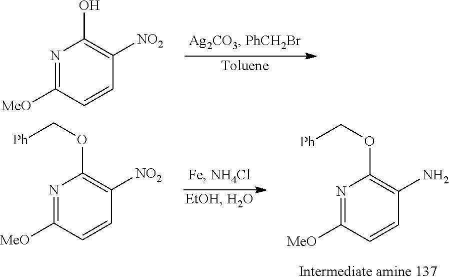

Step 1: In a microwavable glass reaction vessel equipped with a magnetic stirrer was suspended 6-methoxy-3-nitropyridin-2-ol (1 equiv, Combi-Blocks), silver carbonate (1.2 equiv, Sigma-Aldrich), and benzyl bromide (1.2 equiv, Sigma-Aldrich) in toluene (0.10 M). The vessel was then tightly sealed before it was heated at 130° C. in a microwave reactor for 1 h. After cooling to RT, the reaction suspension was filtered through a bed of celite and the insolubles were washed further with EtOAc. The filtrate thus obtained was washed sequentially with water and brine, dried over MgSO₄, and filtered. Concentration of the filtrate in vacuo afforded crude 2-(benzyloxy)-6-methoxy-3-nitropyridine as a brown oil.

Step 2: In a dried, round-bottom flask equipped with a magnetic stirrer was combined 2-(benzyloxy)-6-methoxy-3-nitropyridine (1 equiv) from the previous step and ammonium chloride (5 equiv, Fisher Scientific) in a 2:1 (v/v) solution of ethanol and water (0.07 M). To this solution was then added iron powder (5 equiv, Sigma-Aldrich) in one rapid portion and the resulting grey suspension was heated at 80° C. under a nitrogen atmosphere for 12 h. The reaction suspension was then cooled to RT, filtered through a bed of celite, and the insolubles washed with DCM. The filtrate thus obtained was then diluted with water and extracted further with DCM. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded the title compound as a thick, brown oil (43% yield over two steps).

### Intermediate amine 139: Preparation of 2-(2,2-difluoroethoxy)-6-isopropoxypyridin-3-amine

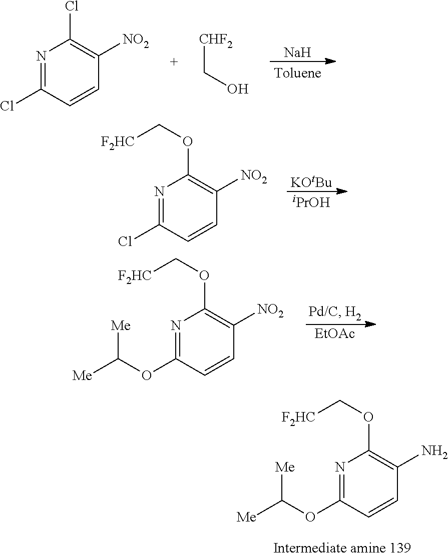

Step 1: In a dried, round-bottom flask equipped with a magnetic stirrer was combined 2,6-dichloro-3-nitropyridine (1 equiv, Combi-Blocks) and 2,2-difluoroethan-1-ol (1.2 equiv, Combi-Blocks) in toluene (0.18 M). To this was then added sodium hydride (60% w/w dispersion in paraffin oil, 1.3 equiv, Sigma-Aldrich) in one rapid portion and the resulting mixture was stirred at RT for 16 h. The reaction was then quenched with water and extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: Hex→5:1 (v/v) Hex: EtOAc) afforded 6-chloro-2-(2,2-difluoroethoxy)-3-nitropyridine as a white solid (16% yield).

Step 2: In a dried, round-bottom flask equipped with a magnetic stirrer was dissolved 6-chloro-2-(2,2-difluoroethoxy)-3-nitropyridine (1 equiv) from the previous step in isopropanol (0.11 M). To this was then added potassium tert-butoxide (1.3 equiv, Sigma-Aldrich) in one rapid portion and the resulting mixture was stirred at RT for 30 min. The reaction was then quenched with water and extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: Hex→5:1 (v/v) Hex: EtOAc) afforded 2-(2,2-difluoroethoxy)-6-isopropoxy-3-nitropyridine as a yellow solid (34% yield).

Step 3: In a dried, round-bottom flask equipped with a magnetic stirrer was dissolved 2-(2,2-difluoroethoxy)-6-isopropoxy-3-nitropyridine (1 equiv) from the previous step in EtOAc (0.22 M). The resulting solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 16 h. The reaction was subsequently quenched with dichloromethane and filtered through a bed of dichloromethane-wetted celite. Concentration of the filtrate thus obtained in vacuo afforded the title compound as a yellow solid (83% yield).

### Intermediate amine 142: Preparation of 2-(difluoromethoxy)-6-(methoxy-d₃)pyridin-3-amine

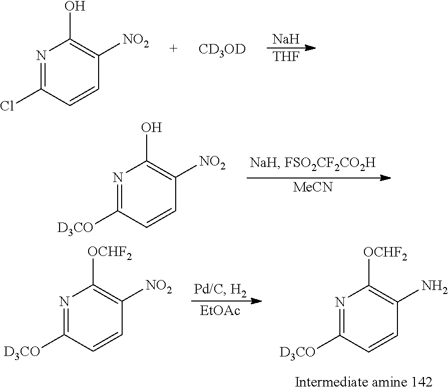

Step 1: In a dried, round-bottom flask equipped with a magnetic stirrer was combined 6-chloro-3-nitropyridin-2-ol (1.1 equiv, Combi-Blocks) and methanol-d₄ (18.5 equiv, Sigma-Aldrich) in anhydrous THE (0.1 M). To this was added sodium hydride (60% w/w dispersion in paraffin oil, 4 equiv, Sigma-Aldrich) in one rapid portion at 0° C., and the resulting mixture was stirred at 0° C. for 5 min and then warmed to RT over 2 h. The crude reaction mixture was then diluted with water and carefully neutralized with the addition of 1 M aq. HCl. The aqueous layer was separated and back extracted with EtOAc. The combined organic extracts were washed further with water and brine, dried over MgSO₄, and filtered. Concentration of the filtrate thus obtained in vacuo afforded crude 6-(methoxy-d₃)-3-nitropyridin-2-ol as a colorless oil.

Step 2: In a dried, round-bottom flask equipped with a magnetic stirrer was suspended 6-(methoxy-d₃)-3-nitropyridin-2-ol (1 equiv) from the previous step in acetonitrile (0.10 M). To this was added sodium hydride (60% w/w dispersion in paraffin oil, 2.8 equiv, Sigma-Aldrich) in one rapid portion and the resulting mixture was stirred at RT for 10 min to afford a brownish, yellow suspension. Then, 2,2-difluoro-2-(fluorosulfonyl)acetic acid (1.8 equiv, Sigma-Aldrich) was added neat and dropwise over a period of 5 min, during which time a mild exotherm was observed. After 16 h of stirring, another aliquot of 2,2-difluoro-2-(fluorosulfonyl)acetic acid (1.8 equiv, Sigma-Aldrich) was added neat and dropwise over a period of 5 min. After another 3 h of stirring at RT, the crude reaction mixture was carefully quenched with water, and then diluted with a 1:1 (v/v) solution of ethyl acetate and hexanes. The organic layer was then separated and washed sequentially with saturated aq. NaHCO₃, water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 2-(difluoromethoxy)-6-(methoxy-d₃)-3-nitropyridine as a yellow solid (62% yield over two steps).

Step 3: In a dried, round-bottom flask equipped with a magnetic stirrer was dissolved 2-(difluoromethoxy)-6-(methoxy-d₃)-3-nitropyridine (1 equiv) from the previous step in ethyl acetate (0.076 M). The resulting yellow solution was then deoxygenated via subsurface purging with nitrogen for 10 min before palladium (10% w/w over activated carbon, dry, 0.1 equiv, Sigma-Aldrich) was added in one rapid portion. The resulting black suspension was then subsurface purged with hydrogen for 10 min before it was stirred under a static hydrogen atmosphere (maintained with a balloon) at RT for 3 h. The reaction was subsequently diluted with EtOAc and filtered through a bed of dichloromethane-wetted celite. The insolubles were washed further with EtOAc. Concentration of the filtrate thus obtained in vacuo afforded the title compound as a reddish, brown solid (>99% yield).

### Intermediate amine 154: Preparation of 6-(difluoromethyl)-4-isopropoxypyridin-3-amine

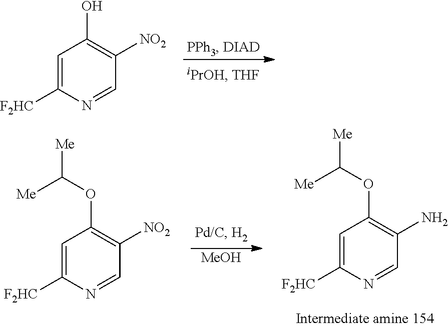

Step 1: In a dried, round-bottom flask equipped with a magnetic stirrer was combined 2-(difluoromethyl)-5-nitropyridin-4-ol (1 equiv, Matrix Scientific), triphenylphosphine (1.5 equiv, Combi-Blocks), and anhydrous isopropanol (3 equiv) in THF (0.11 M). To this was then added, at 0° C., diisopropyl azodicarboxylate (1.5 equiv, Sigma-Aldrich) neat and dropwise over a period of 5 min. After completion of addition, the resulting yellow suspension was allowed to warm to RT over 10 min and stirred at RT for an additional 1 h. The reaction was then quenched with the addition of water and extracted with dichloromethane. The combined organic extracts were washed further with brine, dried over Na₂SO₄, filtered, and the filtrate concentrated in vacuo. Purification of the crude product thus obtained by way of column chromatography (SiO₂, 5:1 (v/v) Hex: EtOAc) afforded 2-(difluoromethyl)-4-isopropoxy-5-nitropyridine as a yellow oil (74% yield).

Step 2: In a dried, round-bottom flask equipped with a magnetic stirrer was dissolved 2-(difluoromethyl)-4-isopropoxy-5-nitropyridine (1 equiv) from the previous step in methanol (0.10 M). The resulting solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 1 h. The reaction was subsequently quenched with dichloromethane and filtered through a bed of dichloromethane-wetted celite. Concentration of the filtrate concentrated in vacuo afforded the title compound as a yellow oil (74% yield).

The following amine was prepared in an analogous fashion to Intermediate amine 154, but substituting isopropanol in step 1 with the requisite, commercially available alcohol.

| Starting Material | Product |
|---|---|
| CD₃OD Sigma-Aldrich | 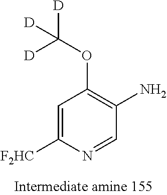 [SMILES: [2H]C([2H])([2H])Oc1cc(C(F)F)ncc1N] |

### Intermediate amine 165: Preparation of 4-chloro-2-(2,2-difluoroethoxy)-5-fluoroaniline

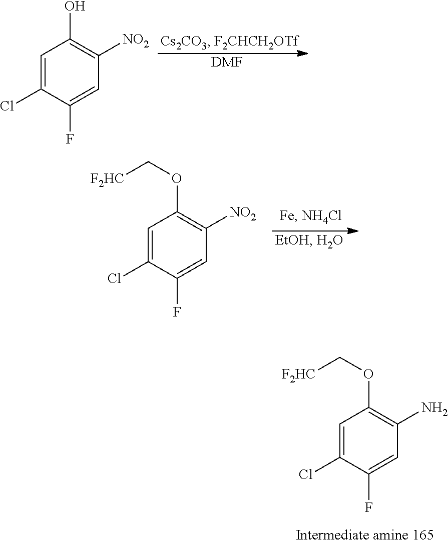

Step 1: In a dried, round-bottom flask equipped with a magnetic stirrer was suspended 5-chloro-4-fluoro-2-nitrophenol (1 equiv, Combi-Blocks), cesium carbonate (3 equiv, Sigma-Aldrich), and 2,2-difluoroethyl triflate (2 equiv, Sigma-Aldrich) in DMF (0.13 M). The reaction mixture stirred at RT for 2 h before it was diluted with water and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded 1-chloro-5-(2,2-difluoroethoxy)-2-fluoro-4-nitrobenzene as a yellow solid (97% yield).

Step 2: In a dried, round-bottom flask equipped with a magnetic stirrer was combined 1-chloro-5-(2,2-difluoroethoxy)-2-fluoro-4-nitrobenzene (1 equiv) from the previous step and ammonium chloride (10 equiv, Fisher Scientific) in a 1:1 (v/v) solution of ethanol and water (0.12 M). To this solution was then added iron powder (10 equiv, Sigma-Aldrich) in one rapid portion and the resulting grey suspension was heated at 80° C. under a nitrogen atmosphere for 1 h. The reaction suspension was then cooled to RT, filtered through a bed of celite, and the insolubles washed with DCM. The filtrate thus obtained was then diluted with water and extracted further with DCM. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded the title compound as a yellow oil (89% yield).

The following amine was prepared in an analogous fashion to Intermediate amine 165, but substituting 2,2-difluoroethyl triflate in step 1 with the requisite, commercially available alkyl halide.

| Starting Material | Product |
|---|---|
| MeI Sigma-Aldrich | 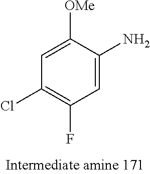 [SMILES: COc1cc(Cl)c(F)cc1N] |

### Intermediate amine 166: Preparation of (S)-2-((1-methoxypropan-2-yl)oxy)-6-methylpyridin-3-amine

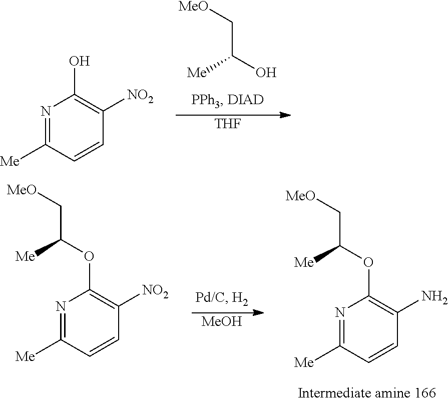

Step 1: In a dried, round-bottom flask equipped with a magnetic stirrer was combined 6-methyl-3-nitropyridin-2-ol (1 equiv, Combi-Blocks), triphenylphosphine (1.5 equiv, Combi-Blocks), and (R)-1-methoxypropan-2-ol (1 equiv, Enamine) in THF (0.42 M). To this was then added, at 0° C., diisopropyl azodicarboxylate (1.5 equiv, Sigma-Aldrich) neat and dropwise over a period of 5 min. After completion of addition, the resulting yellow suspension was allowed to warm to RT over 10 min and stirred at RT for an additional 1 h. The reaction was then quenched with the addition of water and extracted with EtOAc. The combined organic extracts were washed further with brine, dried over Na₂SO₄, filtered, and the filtrate concentrated in vacuo. Purification of the crude product thus obtained by way of column chromatography (SiO₂, 8:1 (v/v) Hex: EtOAc) afforded (S)-2-((1-methoxypropan-2-yl)oxy)-6-methyl-3-nitropyridine as a yellow oil (74% yield).

Step 2: In a dried, round-bottom flask equipped with a magnetic stirrer was dissolved (S)-2-((1-methoxypropan-2-yl)oxy)-6-methyl-3-nitropyridine (1 equiv) from the previous step in methanol (0.53 M). The resulting solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 1 h. The reaction was subsequently quenched with dichloromethane and filtered through a bed of dichloromethane-wetted celite. Concentration of the filtrate concentrated in vacuo afforded the title compound as a yellow oil (71% yield).

The following amine was prepared in an analogous fashion to Intermediate amine 166, but substituting 6-methyl-3-nitropyridin-2-ol in step 1 with the requisite, commercially available (hetero)aryl alcohol.

| Starting Material | Product |
|---|---|
| 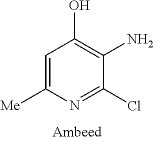 | 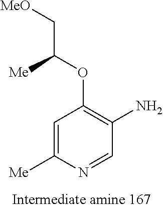 [SMILES: COC[C@H](C)Oc1cc(C)ncc1N] |

### Intermediate amine 168: Preparation of 2-(2,2-difluoroethoxy)-6-(methoxymethyl)pyridin-3-amine

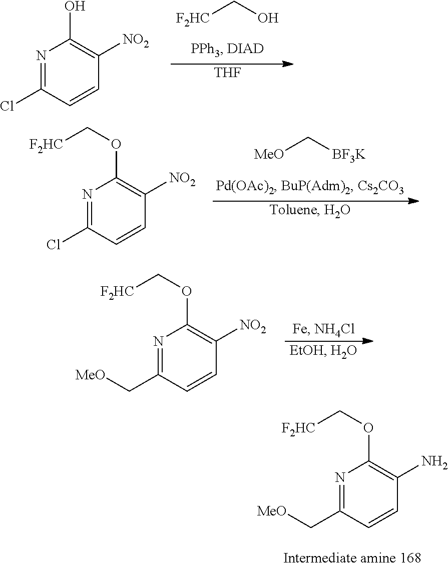

Step 1: In a dried, round-bottom flask equipped with a magnetic stirrer was combined 6-chloro-3-nitropyridin-2-ol (1 equiv, Combi-Blocks), triphenylphosphine (1.5 equiv, Combi-Blocks), and 2,2-difluoroethan-1-ol (1.1 equiv, Combi-Blocks) in THE (0.38 M). To this was then added, at 0° C., diisopropyl azodicarboxylate (1.5 equiv, Sigma-Aldrich) neat and dropwise over a period of 5 min. After completion of addition, the resulting yellow suspension was allowed to warm to RT over 10 min and stirred at RT for an additional 2 h. The reaction was then quenched with the addition of water and extracted with EtOAc. The combined organic extracts were washed further with brine, dried over Na₂SO₄, filtered, and the filtrate concentrated in vacuo. Purification of the crude product thus obtained by way of column chromatography (SiO₂, 5:1 (v/v) Hex: EtOAc) afforded 6-chloro-2-(2,2-difluoroethoxy)-3-nitropyridine as a yellow oil (73% yield).

Step 2: In a thick-walled glass reaction vessel equipped with a magnetic stirrer and a Teflon screwcap was combined 6-chloro-2-(2,2-difluoroethoxy)-3-nitropyridine (1 equiv) from the previous step, potassium methoxymethyltrifluoroborate (1.2 equiv, Combi-Blocks), palladium(II) acetate (0.2 equiv, Sigma-Aldrich), di(1-adamantyl)-n-butylphosphine (0.2 equiv, Sigma-Aldrich), and cesium carbonate (3 equiv, Sigma-Aldrich) in a 10:1 (v/v) solution of toluene and water (0.034 M). The resulting mixture was deoxygenated via subsurface purging with nitrogen for 10 min before the reaction vessel was tightly sealed and heated at 90° C. for 72 h. The now dark brown suspension was cooled to RT, diluted with water, and extracted with EtOAc. The combined organic extracts were washed further with brine, dried over Na₂SO₄, filtered, and the filtrate concentrated in vacuo. Purification of the crude product thus obtained by way of column chromatography (SiO₂, 5:1 (v/v) Hex: EtOAc) afforded 2-(2,2-difluoroethoxy)-6-(methoxymethyl)-3-nitropyridine as a yellow oil (37% yield).

Step 3: In a dried, round-bottom flask equipped with a magnetic stirrer was combined 2-(2,2-difluoroethoxy)-6-(methoxymethyl)-3-nitropyridine (1 equiv) from the previous step and ammonium chloride (10 equiv, Fisher Scientific) in a 1:1 (v/v) solution of ethanol and water (0.097 M). To this solution was then added iron powder (10 equiv, Sigma-Aldrich) in one rapid portion and the resulting grey suspension was heated at 80° C. under a nitrogen atmosphere for 2 h. The reaction suspension was then cooled to RT, filtered through a bed of celite, and the insolubles washed with EtOAc. The filtrate thus obtained was then diluted with water and extracted further with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded the title compound as a yellow oil (57% yield).

### Intermediate amine 169: Preparation of 6-(benzyloxy)-2-(difluoromethoxy)pyridin-3-amine

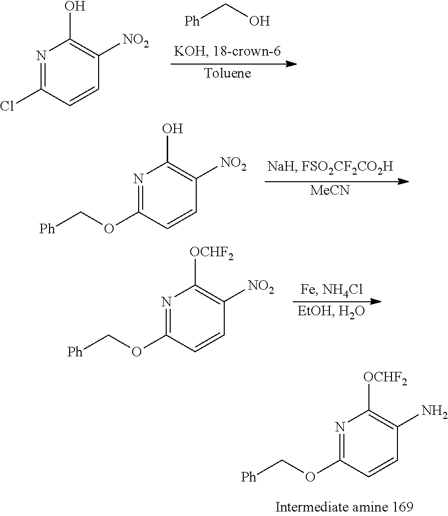

Step 1: In a dried, round-bottom flask equipped with a magnetic stirrer was combined 6-chloro-3-nitropyridin-2-ol (1 equiv, Combi-Blocks), benzyl alcohol (1.1 equiv, Sigma-Aldrich), freshly powdered potassium hydroxide (3 equiv, J. T Baker), and 18-crown-6 (0.03 equiv, Sigma-Aldrich) in toluene (0.19 M). The resulting suspension was heated at 80° C. for 2 h. The reaction was then quenched with the addition of 1 M aq. HCl until a pH of ˜4 and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 2:1 (v/v) Hex: EtOAc) afforded 6-(benzyloxy)-3-nitropyridin-2-ol as a yellow oil (73% yield).

Step 2: In a dried, round-bottom flask equipped with a magnetic stirrer was suspended 6-(benzyloxy)-3-nitropyridin-2-ol (1 equiv) from the previous step in acetonitrile (0.10 M). To this was then added sodium hydride (60% w/w dispersion in paraffin oil, 2.8 equiv, Sigma-Aldrich) in one rapid portion and the resulting mixture was stirred at RT for 10 min to afford a brownish, yellow suspension. Then, 2,2-difluoro-2-(fluorosulfonyl)acetic acid (1.8 equiv, Sigma-Aldrich) was added neat and dropwise over a period of 5 min, during which time a mild exotherm was observed. After 16 h of stirring, another aliquot of 2,2-difluoro-2-(fluorosulfonyl)acetic acid (1.8 equiv, Sigma-Aldrich) was added neat and dropwise over a period of 5 min. After another 48 h of stirring at RT, the crude reaction mixture was carefully quenched with water, and then diluted with a 1:1 (v/v) solution of ethyl acetate and hexanes. The organic layer was then separated and washed sequentially with saturated aq. NaHCO₃, water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 6-(benzyloxy)-2-(difluoromethoxy)-3-nitropyridine as a yellow oil (64% yield).

Step 3: In a dried, round-bottom flask equipped with a magnetic stirrer was combined 6-(benzyloxy)-2-(difluoromethoxy)-3-nitropyridine (1 equiv) from the previous step and ammonium chloride (10 equiv, Fisher Scientific) in a 1:1 (v/v) solution of ethanol and water (0.11 M). To this solution was then added iron powder (10 equiv, Sigma-Aldrich) in one rapid portion and the resulting grey suspension was heated at 90° C. under a nitrogen atmosphere for 1 h. The reaction suspension was then cooled to RT, filtered through a bed of celite, and the insolubles washed with EtOAc. The filtrate thus obtained was then diluted with water and extracted further with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex→EtOAc) afforded the title compound as a yellow oil (57% yield).

### Intermediate amine 170: Preparation of 5-chloro-2-methoxy-4-methylaniline

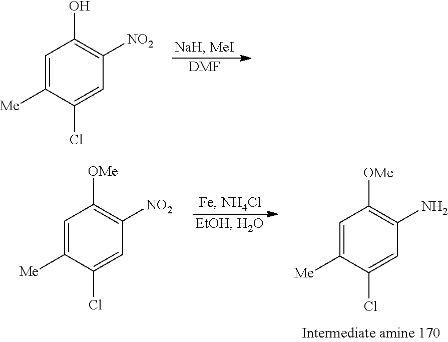

Step 1: In a dried, round-bottom flask equipped with a magnetic stirrer was dissolved 4-chloro-5-methyl-2-nitrophenol (1 equiv, Combi-Blocks) in DMF (0.76 M). To this was added sodium hydride (60% w/w dispersion in paraffin oil, 3 equiv, Sigma-Aldrich) portionwise at 0° C., and the resulting mixture was stirred at 0° C. for 30 min. Finally, to this mixture was then added, at 0° C., iodomethane (3 equiv, Sigma-Aldrich) neat and dropwise over a period of 2 min. The reaction mixture was allowed to warm to RT and stirred at RT for 2 h. The reaction was then carefully quenched at 0° C. with the dropwise addition of water and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 5:1 (v/v) Hex: EtOAc) afforded 1-chloro-4-methoxy-2-methyl-5-nitrobenzene as a yellow oil (82% yield).

Step 2: In a dried, round-bottom flask equipped with a magnetic stirrer was combined 1-chloro-4-methoxy-2-methyl-5-nitrobenzene (1 equiv) from the previous step and ammonium chloride (10 equiv, Fisher Scientific) in a 1:1 (v/v) solution of ethanol and water (0.11 M). To this solution was then added iron powder (10 equiv, Sigma-Aldrich) in one rapid portion and the resulting grey suspension was heated at 80° C. under a nitrogen atmosphere for 4 h. The reaction suspension was then cooled to RT, filtered through a bed of celite, and the insolubles washed with EtOAc. The filtrate thus obtained was then diluted with water and extracted further with EtOAc. The combined organic extracts were washed further with water and brine, dried over Na₂SO₄, filtered, and the filtrate concentrated in vacuo. Purification of the crude product thus obtained by way of column chromatography (SiO₂, 4:1 (v/v) Hex: EtOAc) afforded the title compound as a yellow solid (91% yield).

### Example 1: Preparation of tert-butyl 4-(3-(2-(difluoromethoxy)-6-methylpyridin-3-yl)-1-(2-isopropylphenyl)ureido)piperidine-1-carboxylate

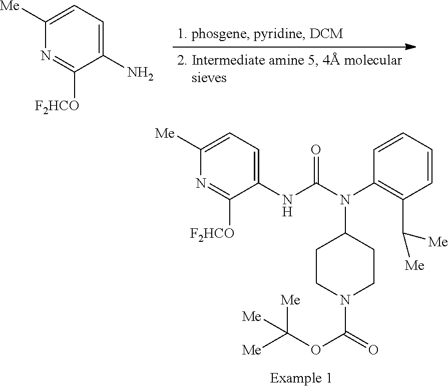 [SMILES: Cc1ccc(N)c(OC(F)F)n1 ; Cc1ccc(NC(=O)N(c2ccccc2C(C)C)C2CCN(C(=O)OC(C)(C)C)CC2)c(OC(F)F)n1]

In a dried, round-bottom flask equipped with a magnetic stirrer was combined 2-(difluoromethoxy)-6-methylpyridin-3-amine (1 equiv, synthesized as per WO2019/234115) and pyridine (3 equiv, Sigma-Aldrich) in anhydrous dichloromethane (0.084 M). To this was then added phosgene (15% w/w solution in toluene, 1.5 equiv, Sigma-Aldrich) dropwise at RT and the resulting solution was stirred at RT for 30 min. The volatiles were then removed in vacuo and the crude (2-(difluoromethoxy)-6-methylpyridin-3-yl)carbamic chloride thus obtained was re-taken up in anhydrous dichloromethane (0.084 M). This solution was then added dropwise at RT to another dichloromethane suspension (0.084 M) of Intermediate amine 5 (1 equiv), pyridine (3 equiv, Sigma-Aldrich), and freshly activated 4 Å molecular sieves. The resulting mixture was stirred at RT for 16 h before the reaction was quenched with water. The aqueous layer was separated and back extracted with DCM. The combined organic extracts were dried over MgSO₄, filtered, and the filtrate concentrated in vacuo. Purification of the crude product thus obtained by way of column chromatography (SiO₂, gradient elution: Hex→EtOAc) afforded the title compound as a white solid (6400 yield). LCMS: m/z=541.1 [M+Na]⁺; ¹H NMR (DMSO-d₆): δ=8.30 (d, J=8.0 Hz, 1H), 7.57 (dd, J=7.5, 1.0 Hz, 1H), 7.50 (t, J=7.5 Hz, 1H), 7.44 (t, J=72.5 Hz, 1H), 7.35 (td, J=7.5, 1.5 Hz, 1H), 7.26 (dd, J=7.5, 1.0 Hz, 1H), 7.04 (d, J=8.0 Hz, 1H), 6.31 (s, 1H), 4.36 (tt, J=12.0, 3.5 Hz, 1H), 4.04˜3.86 (m, 2H), 3.09 (sept, J=7.0 Hz, 1H), 2.91˜2.66 (m, 2H), 2.31 (s, 3H), 1.97 (br d, J=12.0 Hz, 1H), 1.73 (br d, J=12.0 Hz, 1H), 1.46˜1.40 (m, 1H), 1.34 (s, 9H), 1.20 (d, J=7.0 Hz, 3H), 1.08 (d, J=7.0 Hz, 3H), 1.07˜1.02 (in, 1H).

The following examples were prepared in an analogous fashion to Example 1, but substituting Intermediate amine 5 with the requisite amine. In Example 31, Example 32, Example 33, Example 34, Example 35, and Example 36, phosgene (1.5 equiv) was also substituted with triphosgene (0.5 equiv, Sigma-Aldrich).

| | | | LCMS |
|---|---|---|---|
| Example | Starting Amine | Structure | (m/z) |
| 2 | 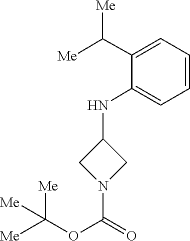 [SMILES: CC(C)c1ccccc1NC1CN(C(=O)OC(C)(C)C)C1] | 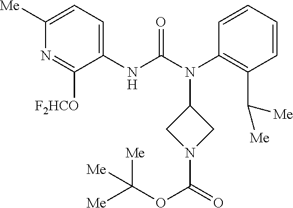 [SMILES: Cc1ccc(NC(=O)N(c2ccccc2C(C)C)C2CN(C(=O)OC(C)(C)C)C2)c(OC(F)F)n1] | 491.1 [M + H]⁺ |
| | Intermediate | tert-butyl 3-(3-(2-(difluoromethoxy)-6- | |
| | amine 1 | methylpyridin-3-yl)-1-(2- | |
| | | isopropylphenyl)ureido)azetidine-1- | |
| | | carboxylate | |
| 3 | 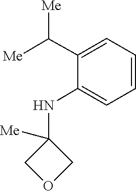 [SMILES: CC(C)c1ccccc1NC1(C)COC1] | 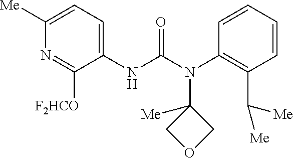 [SMILES: Cc1ccc(NC(=O)N(c2ccccc2C(C)C)C2(C)COC2)c(OC(F)F)n1] | 405.9 [M + H]⁺ |
| | Intermediate | 3-(2-(difluoromethoxy)-6-methylpyridin-3- | |
| | amine 2 | yl)-1-(2-isopropylphenyl)-1-(3-methyloxetan- | |
| | | 3-yl)urea | |
| 4 | 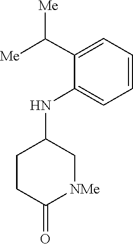 [SMILES: CC(C)c1ccccc1NC1CCC(=O)N(C)C1] | 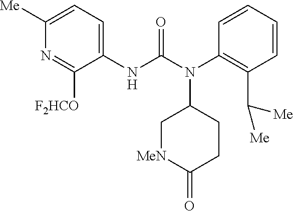 [SMILES: Cc1ccc(NC(=O)N(c2ccccc2C(C)C)C2CCC(=O)N(C)C2)c(OC(F)F)n1] | 447.1 [M + H]⁺ |
| | Intermediate | 3-(2-(difluoromethoxy)-6-methylpyridin-3- | |
| | amine 6 | yl)-1-(2-isopropylphenyl)-1-(1-methyl-6- | |
| | | oxopiperidin-3-yl)urea | |
| 5 | 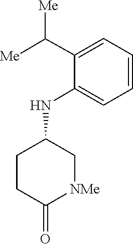 [SMILES: CC(C)c1ccccc1N[C@H]1CCC(=O)N(C)C1] | 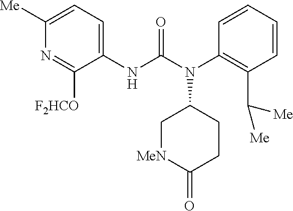 [SMILES: Cc1ccc(NC(=O)N(c2ccccc2C(C)C)[C@@H]2CCC(=O)N(C)C2)c(OC(F)F)n1] | 447.1 [M + H]⁺ |
| | Intermediate | (R)-3-(2-(difluoromethoxy)-6-methylpyridin- | |
| | amine 7 | 3-yl)-1-(2-isopropylphenyl)-1-(1-methyl-6- | |
| | | oxopiperidin-3-yl)urea | |
| 6 | 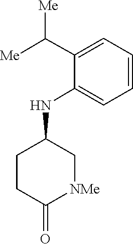 [SMILES: CC(C)c1ccccc1N[C@@H]1CCC(=O)N(C)C1] | 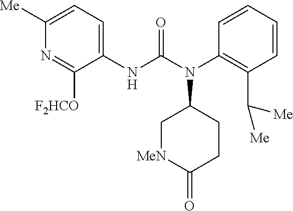 [SMILES: Cc1ccc(NC(=O)N(c2ccccc2C(C)C)[C@H]2CCC(=O)N(C)C2)c(OC(F)F)n1] | 447.1 [M + H]⁺ |
| | Intermediate | (S)-3-(2-(difluoromethoxy)-6-methylpyridin- | |
| | amine 8 | 3-yl)-1-(2-isopropylphenyl)-1-(1-methyl-6- | |
| | | oxopiperidin-3-yl)urea | |
| 7 | 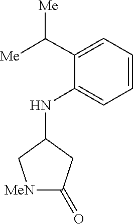 [SMILES: CC(C)c1ccccc1NC1CC(=O)N(C)C1] | 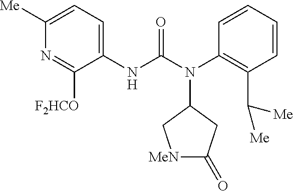 [SMILES: Cc1ccc(NC(=O)N(c2ccccc2C(C)C)C2CC(=O)N(C)C2)c(OC(F)F)n1] | 433.1 [M + H]⁺ |
| | Intermediate | 3-(2-(difluoromethoxy)-6-methylpyridin-3- | |
| | amine 9 | yl)-1-(2-isopropylphenyl)-1-(1-methyl-5- | |
| | | oxopyrrolidin-3-yl)urea | |
| 8 | 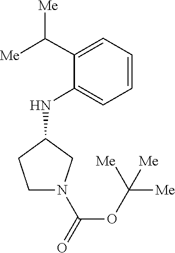 [SMILES: CC(C)c1ccccc1N[C@H]1CCN(C(=O)OC(C)(C)C)C1] | 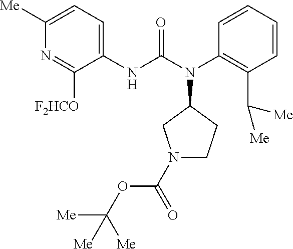 [SMILES: Cc1ccc(NC(=O)N(c2ccccc2C(C)C)[C@H]2CCN(C(=O)OC(C)(C)C)C2)c(OC(F)F)n1] | 527.1 [M + Na]⁺ |
| | Intermediate | tert-butyl (S)-3-(3-(2-(difluoromethoxy)-6- | |
| | amine 10 | methylpyridin-3-yl)-1-(2- | |
| | | isopropylphenyl)ureido)pyrrolidine-1- | |
| | | carboxylate | |
| 9 | 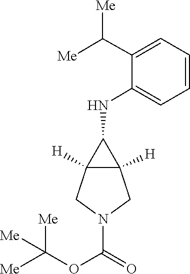 [SMILES: CC(C)c1ccccc1N[C@H]1[C@@H]2CN(C(=O)OC(C)(C)C)C[C@@H]21] | 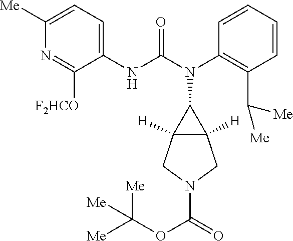 [SMILES: Cc1ccc(NC(=O)N(c2ccccc2C(C)C)[C@H]2[C@@H]3CN(C(=O)OC(C)(C)C)C[C@@H]32)c(OC(F)F)n1] | 539.1 [M + Na]⁺ |
| | Intermediate | tert-butyl (1R,5S,6s)-6-(3-(2- | |
| | amine 3 | (difluoromethoxy)-6-methylpyridin-3-yl)-1- | |
| | | (2-isopropylphenyl)ureido)-3- | |
| | | azabicyclo[3.1.0]hexane-3-carboxylate | |
| 10 | 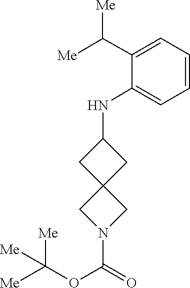 [SMILES: CC(C)c1ccccc1NC1CC2(C1)CN(C(=O)OC(C)(C)C)C2] | 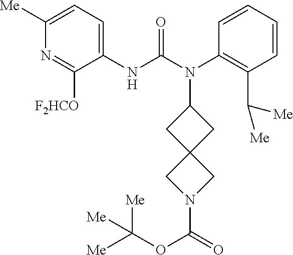 [SMILES: Cc1ccc(NC(=O)N(c2ccccc2C(C)C)C2CC3(C2)CN(C(=O)OC(C)(C)C)C3)c(OC(F)F)n1] | 531.1 [M + H]⁺ |
| | Intermediate | tert-butyl 6-(3-(2-(difluoromethoxy)-6- | |
| | amine 11 | methylpyridin-3-yl)-1-(2- | |
| | | isopropylphenyl)ureido)-2- | |
| | | azaspiro[3.3]heptane-2-carboxylate | |
| 11 | 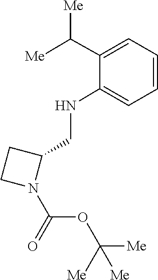 [SMILES: CC(C)c1ccccc1NC[C@H]1CCN1C(=O)OC(C)(C)C] | 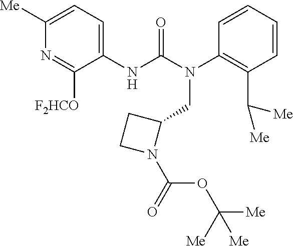 [SMILES: Cc1ccc(NC(=O)N(C[C@H]2CCN2C(=O)OC(C)(C)C)c2ccccc2C(C)C)c(OC(F)F)n1] | 505.0 [M + H]⁺ |
| | Intermediate | tert-butyl (R)-2-((3-(2-(difluoromethoxy)-6- | |
| | amine 12 | methylpyridin-3-yl)-1-(2- | |
| | | isopropylphenyl)ureido)methyl)azetidine-1- | |
| | | carboxylate | |
| 12 | 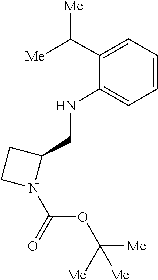 [SMILES: CC(C)c1ccccc1NC[C@@H]1CCN1C(=O)OC(C)(C)C] | 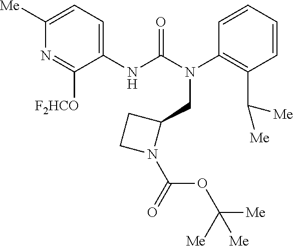 [SMILES: Cc1ccc(NC(=O)N(C[C@@H]2CCN2C(=O)OC(C)(C)C)c2ccccc2C(C)C)c(OC(F)F)n1] | 505.0 [M + H]⁺ |
| | Intermediate | tert-butyl (S)-2-((3-(2-(difluoromethoxy)-6- | |
| | amine 13 | methylpyridin-3-yl)-1-(2- | |
| | | isopropylphenyl)ureido)methyl)azetidine-1- | |
| | | carboxylate | |
| 13 | 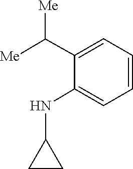 [SMILES: CC(C)c1ccccc1NC1CC1] | 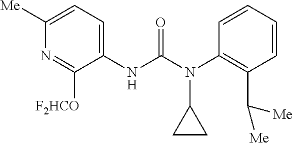 [SMILES: Cc1ccc(NC(=O)N(c2ccccc2C(C)C)C2CC2)c(OC(F)F)n1] | 375.9 [M + H]⁺ |
| | Intermediate | 1-cyclopropyl-3-(2-(difluoromethoxy)-6- | |
| | amine 14 | methylpyridin-3-yl)-1-(2- | |
| | | isopropylphenyl)urea | |
| 14 | 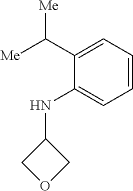 [SMILES: CC(C)c1ccccc1NC1COC1] | 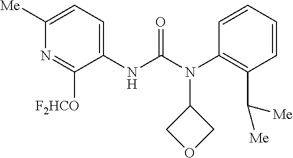 [SMILES: Cc1ccc(NC(=O)N(c2ccccc2C(C)C)C2COC2)c(OC(F)F)n1] | 391.9 [M + H]⁺ |
| | Intermediate | 3-(2-(difluoromethoxy)-6-methylpyridin-3- | |
| | amine 15 | yl)-1-(2-isopropylphenyl)-1-(oxetan-3-yl)urea | |
| 15 | 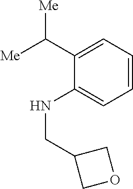 [SMILES: CC(C)c1ccccc1NCC1COC1] | 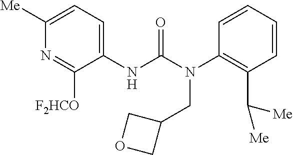 [SMILES: Cc1ccc(NC(=O)N(CC2COC2)c2ccccc2C(C)C)c(OC(F)F)n1] | 406.1 [M + H]⁺ |
| | Intermediate | 3-(2-(difluoromethoxy)-6-methylpyridin-3- | |
| | amine 16 | yl)-1-(2-isopropylphenyl)-1-(oxetan-3- | |
| | | ylmethyl)urea | |
| 16 | 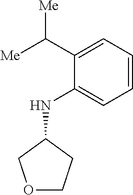 [SMILES: CC(C)c1ccccc1N[C@@H]1CCOC1] | 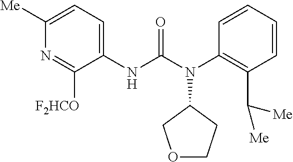 [SMILES: Cc1ccc(NC(=O)N(c2ccccc2C(C)C)[C@@H]2CCOC2)c(OC(F)F)n1] | 405.9 [M + H]⁺ |
| | Intermediate | (R)-3-(2-(difluoromethoxy)-6-methylpyridin- | |
| | amine 17 | 3-yl)-1-(2-isopropylphenyl)-1- | |
| | | (tetrahydrofuran-3-yl)urea | |
| 17 | 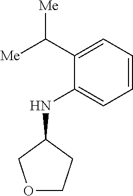 [SMILES: CC(C)c1ccccc1N[C@H]1CCOC1] | 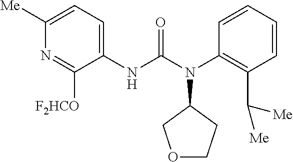 [SMILES: Cc1ccc(NC(=O)N(c2ccccc2C(C)C)[C@H]2CCOC2)c(OC(F)F)n1] | 405.9 [M + H]⁺ |
| | Intermediate | (S)-3-(2-(difluoromethoxy)-6-methylpyridin- | |
| | amine 18 | 3-yl)-1-(2-isopropylphenyl)-1- | |
| | | (tetrahydrofuran-3-yl)urea | |
| 18 | 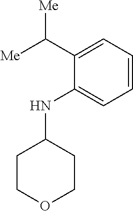 [SMILES: CC(C)c1ccccc1NC1CCOCC1] | 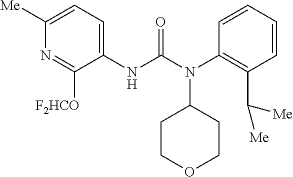 [SMILES: Cc1ccc(NC(=O)N(c2ccccc2C(C)C)C2CCOCC2)c(OC(F)F)n1] | 419.9 [M + H]⁺ |
| | Intermediate | 3-(2-(difluoromethoxy)-6-methylpyridin-3- | |
| | amine 19 | yl)-1-(2-isopropylphenyl)-1-(tetrahydro-2H- | |
| | | pyran-4-yl)urea | |
| 19 | 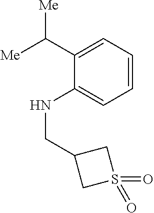 [SMILES: CC(C)c1ccccc1NCC1CS(=O)(=O)C1] | 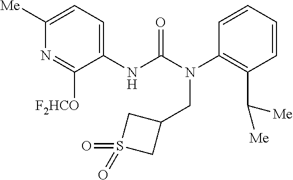 [SMILES: Cc1ccc(NC(=O)N(CC2CS(=O)(=O)C2)c2ccccc2C(C)C)c(OC(F)F)n1] | 454.2 [M + H]⁺ |
| | Intermediate | 3-(2-(difluoromethoxy)-6-methylpyridin-3- | |
| | amine 20 | yl)-1-((1,1-dioxidothietan-3-yl)methyl)-1-(2- | |
| | | isopropylphenyl)urea | |
| 20 | 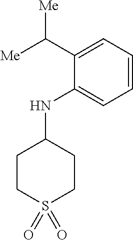 [SMILES: CC(C)c1ccccc1NC1CCS(=O)(=O)CC1] | 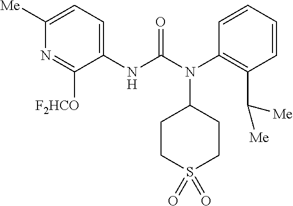 [SMILES: Cc1ccc(NC(=O)N(c2ccccc2C(C)C)C2CCS(=O)(=O)CC2)c(OC(F)F)n1] | 468.0 [M + H]⁺ |
| | Intermediate | 3-(2-(difluoromethoxy)-6-methylpyridin-3- | |
| | amine 21 | yl)-1-(1,1-dioxidotetrahydro-2H-thiopyran-4- | |
| | | yl)-1-(2-isopropylphenyl)urea | |
| 21 | 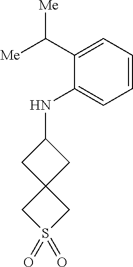 [SMILES: CC(C)c1ccccc1NC1CC2(C1)CS(=O)(=O)C2] | 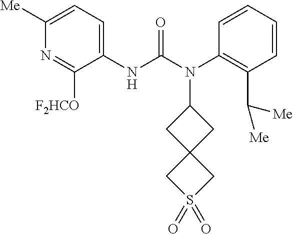 [SMILES: Cc1ccc(NC(=O)N(c2ccccc2C(C)C)C2CC3(C2)CS(=O)(=O)C3)c(OC(F)F)n1] | 480.2 [M + H]⁺ |
| | Intermediate | 3-(2-(difluoromethoxy)-6-methylpyridin-3- | |
| | amine 22 | yl)-1-(2,2-dioxido-2-thiaspiro[3.3]heptan-6- | |
| | | yl)-1-(2-isopropylphenyl)urea | |
| 22 | 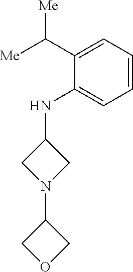 [SMILES: CC(C)c1ccccc1NC1CN(C2COC2)C1] | 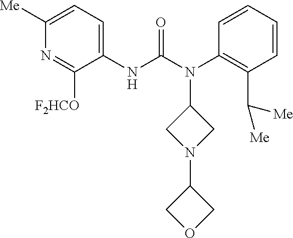 [SMILES: Cc1ccc(NC(=O)N(c2ccccc2C(C)C)C2CN(C3COC3)C2)c(OC(F)F)n1] | 447.2 [M + H]⁺ |
| | Intermediate | 3-(2-(difluoromethoxy)-6-methylpyridin-3- | |
| | amine 24 | yl)-1-(2-isopropylphenyl)-1-(1-(oxetan-3- | |
| | | yl)azetidin-3-yl)urea | |
| 23 | 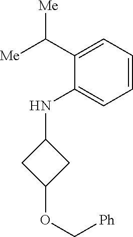 [SMILES: CC(C)c1ccccc1NC1CC(OCc2ccccc2)C1] | 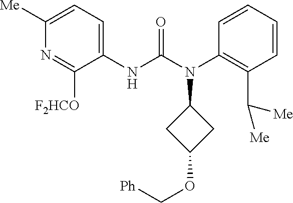 [SMILES: Cc1ccc(NC(=O)N(c2ccccc2C(C)C)[C@H]2C[C@H](OCc3ccccc3)C2)c(OC(F)F)n1] | 496.0 [M + H]⁺ |
| | Intermediate | 1-((1r,3r)-3-(benzyloxy)cyclobutyl)-3-(2- | |
| | amine 69 | (difluoromethoxy)-6-methylpyridin-3-yl)-1- | |
| | | (2-isopropylphenyl)urea | |
| 24 | 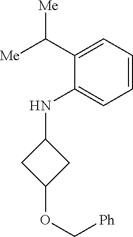 [SMILES: CC(C)c1ccccc1NC1CC(OCc2ccccc2)C1] | 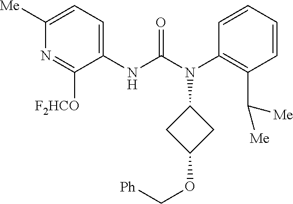 [SMILES: Cc1ccc(NC(=O)N(c2ccccc2C(C)C)[C@H]2C[C@@H](OCc3ccccc3)C2)c(OC(F)F)n1] | 496.0 [M + H]⁺ |
| | Intermediate | 1-((1s,3s)-3-(benzyloxy)cyclobutyl)-3-(2- | |
| | amine 69 | (difluoromethoxy)-6-methylpyridin-3-yl)-1- | |
| | | (2-isopropylphenyl)urea | |
| 25 | 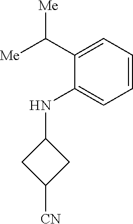 [SMILES: CC(C)c1ccccc1NC1CC(C#N)C1] | 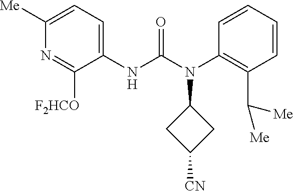 [SMILES: Cc1ccc(NC(=O)N(c2ccccc2C(C)C)[C@H]2C[C@H](C#N)C2)c(OC(F)F)n1] | 415.1 [M + H]⁺ |
| | Intermediate | 1-((1r,3r)-3-cyanocyclobutyl)-3-(2- | |
| | amine 70 | (difluoromethoxy)-6-methylpyridin-3-yl)-1- | |
| | | (2-isopropylphenyl)urea | |
| 26 | 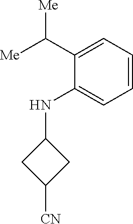 [SMILES: CC(C)c1ccccc1NC1CC(C#N)C1] | 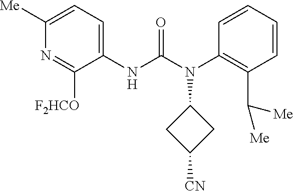 [SMILES: Cc1ccc(NC(=O)N(c2ccccc2C(C)C)[C@H]2C[C@@H](C#N)C2)c(OC(F)F)n1] | 415.1 [M + H]⁺ |
| | Intermediate | 1-((1s,3s)-3-cyanocyclobutyl)-3-(2- | |
| | amine 70 | (difluoromethoxy)-6-methylpyridin-3-yl)-1- | |
| | | (2-isopropylphenyl)urea | |
| 27 | 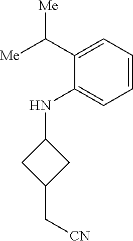 [SMILES: CC(C)c1ccccc1NC1CC(CC#N)C1] | 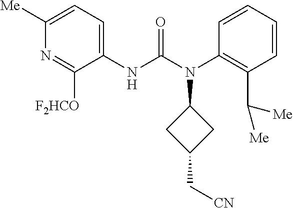 [SMILES: Cc1ccc(NC(=O)N(c2ccccc2C(C)C)[C@H]2C[C@H](CC#N)C2)c(OC(F)F)n1] | 429.1 [M + H]⁺ |
| | Intermediate | 1-((1r,3r)-3-(cyanomethyl)cyclobutyl)-3-(2- | |
| | amine 71 | (difluoromethoxy)-6-methylpyridin-3-yl)-1- | |
| | | (2-isopropylphenyl)urea | |
| 28 | 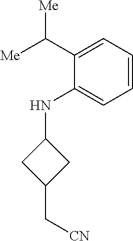 [SMILES: CC(C)c1ccccc1NC1CC(CC#N)C1] | 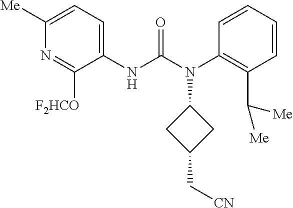 [SMILES: Cc1ccc(NC(=O)N(c2ccccc2C(C)C)[C@H]2C[C@@H](CC#N)C2)c(OC(F)F)n1] | 429.1 [M + H]⁺ |
| | Intermediate | 1-((1r,3s)-3-(cyanomethyl)cyclobutyl)-3-(2- | |
| | amine 71 | (difluoromethoxy)-6-methylpyridin-3-yl)-1- | |
| | | (2-isopropylphenyl)urea | |
| 29 | 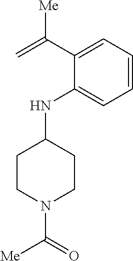 | 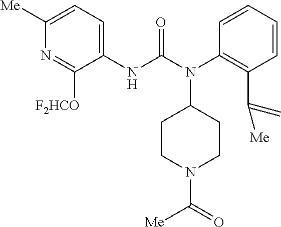 [SMILES: C=C(C)c1ccccc1N(C(=O)Nc1ccc(C)nc1OC(F)F)C1CCN(C(C)=O)CC1] | 459.0 [M + H]⁺ |
| | Intermediate | 1-(1-acetylpiperidin-4-yl)-3-(2- | |
| | amine 77 | (difluoromethoxy)-6-methylpyridin-3-yl)-1- | |
| | | (2-(prop-1-en-2-yl)phenyl)urea | |
| 30 | 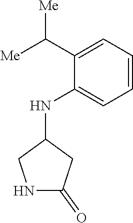 [SMILES: CC(C)c1ccccc1NC1CNC(=O)C1] | 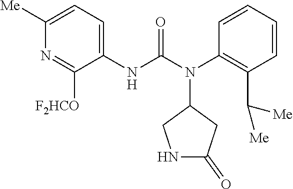 [SMILES: Cc1ccc(NC(=O)N(c2ccccc2C(C)C)C2CNC(=O)C2)c(OC(F)F)n1] | 419.1 [M + H]⁺ |
| | Intermediate | 3-(2-(difluoromethoxy)-6-methylpyridin-3- | |
| | amine 78 | yl)-1-(2-isopropylphenyl)-1-(5-oxopyrrolidin- | |
| | | 3-yl)urea | |
| 31 | 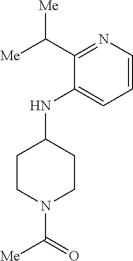 [SMILES: CC(=O)N1CCC(Nc2cccnc2C(C)C)CC1] | 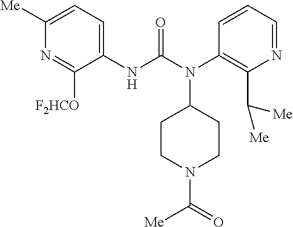 [SMILES: CC(=O)N1CCC(N(C(=O)Nc2ccc(C)nc2OC(F)F)c2cccnc2C(C)C)CC1] | 462.2 [M + H]⁺ |
| | Intermediate | 1-(1-acetylpiperidin-4-yl)-3-(2- | |
| | amine 75 | (difluoromethoxy)-6-methylpyridin-3-yl)-1- | |
| | | (2-isopropylpyridin-3-yl)urea | |
| 32 | 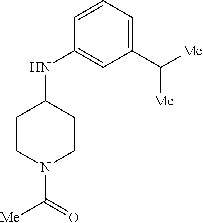 [SMILES: CC(=O)N1CCC(Nc2cccc(C(C)C)c2)CC1] | 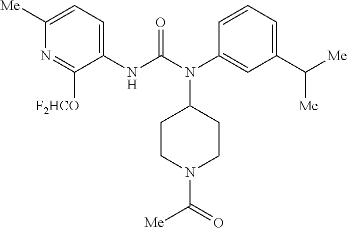 [SMILES: CC(=O)N1CCC(N(C(=O)Nc2ccc(C)nc2OC(F)F)c2cccc(C(C)C)c2)CC1] | 461.2 [M + H]⁺ |
| | Intermediate | 1-(1-acetylpiperidin-4-yl)-3-(2- | |
| | amine 53 | (difluoromethoxy)-6-methylpyridin-3-yl)-1- | |
| | | (3-isopropylphenyl)urea | |
| 33 | 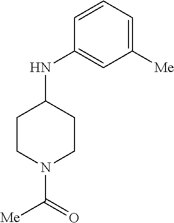 [SMILES: CC(=O)N1CCC(Nc2cccc(C)c2)CC1] | 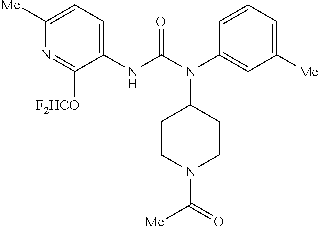 [SMILES: CC(=O)N1CCC(N(C(=O)Nc2ccc(C)nc2OC(F)F)c2cccc(C)c2)CC1] | 433.2 [M + H]⁺ |
| | Intermediate | 1-(1-acetylpiperidin-4-yl)-3-(2- | |
| | amine 54 | (difluoromethoxy)-6-methylpyridin-3-yl)-1- | |
| | | (m-tolyl)urea | |
| 34 | 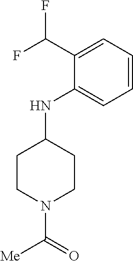 [SMILES: CC(=O)N1CCC(Nc2ccccc2C(F)F)CC1] | 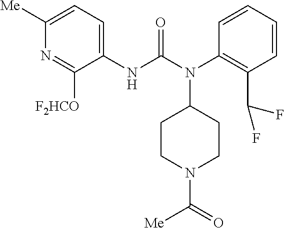 [SMILES: CC(=O)N1CCC(N(C(=O)Nc2ccc(C)nc2OC(F)F)c2ccccc2C(F)F)CC1] | 469.2 [M + H]⁺ |
| | Intermediate | 1-(1-acetylpiperidin-4-yl)-3-(2- | |
| | amine 56 | (difluoromethoxy)-6-methylpyridin-3-yl)-1- | |
| | | (2-(difluoromethyl)phenyl)urea | |
| 35 | 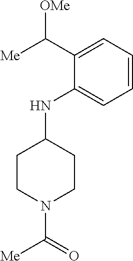 [SMILES: COC(C)c1ccccc1NC1CCN(C(C)=O)CC1] | 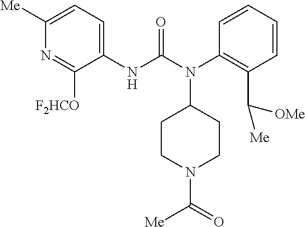 [SMILES: COC(C)c1ccccc1N(C(=O)Nc1ccc(C)nc1OC(F)F)C1CCN(C(C)=O)CC1] | 477.3 [M + H]⁺ |
| | Intermediate | 1-(1-acetylpiperidin-4-yl)-3-(2- | |
| | amine 57 | (difluoromethoxy)-6-methylpyridin-3-yl)-1- | |
| | | (2-(1-methoxyethyl)phenyl)urea | |
| 36 | 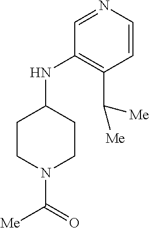 [SMILES: CC(=O)N1CCC(Nc2cnccc2C(C)C)CC1] | 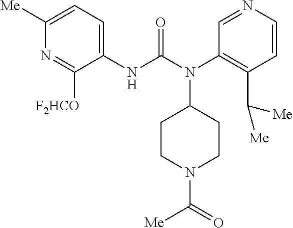 [SMILES: CC(=O)N1CCC(N(C(=O)Nc2ccc(C)nc2OC(F)F)c2cnccc2C(C)C)CC1] | 462.2 [M + H]⁺ |
| | Intermediate | 1-(1-acetylpiperidin-4-yl)-3-(2- | |
| | amine 76 | (difluoromethoxy)-6-methylpyridin-3-yl)-1- | |
| | | (4-isopropylpyridin-3-yl)urea | |
| 236 | 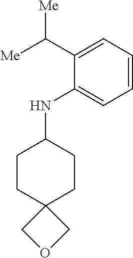 [SMILES: CC(C)c1ccccc1NC1CCC2(CC1)COC2] | 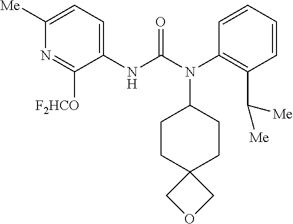 [SMILES: Cc1ccc(NC(=O)N(c2ccccc2C(C)C)C2CCC3(CC2)COC3)c(OC(F)F)n1] | 459.8 [M + H]⁺ |
| | Intermediate | 3-(2-(difluoromethoxy)-6-methylpyridin-3- | |
| | amine 126 | yl)-1-(2-isopropylphenyl)-1-(2- | |
| | | oxaspiro[3.5]nonan-7-yl)urea | |
| 237 | 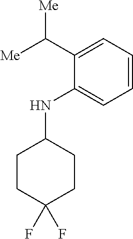 [SMILES: CC(C)c1ccccc1NC1CCC(F)(F)CC1] | 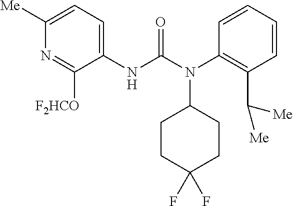 [SMILES: Cc1ccc(NC(=O)N(c2ccccc2C(C)C)C2CCC(F)(F)CC2)c(OC(F)F)n1] | 453.8 [M + H]⁺ |
| | Intermediate | 1-(4,4-difluorocyclohexyl)-3-(2- | |
| | amine 127 | (difluoromethoxy)-6-methylpyridin-3-yl)-1- | |
| | | (2-isopropylphenyl)urea | |
| 238 | 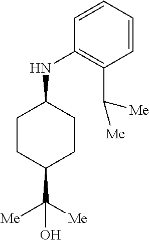 [SMILES: CC(C)c1ccccc1N[C@H]1CC[C@@H](C(C)(C)O)CC1] | 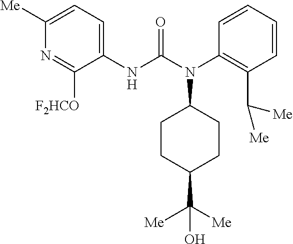 [SMILES: Cc1ccc(NC(=O)N(c2ccccc2C(C)C)[C@H]2CC[C@@H](C(C)(C)O)CC2)c(OC(F)F)n1] | 475.9 [M + H]⁺ |
| | Intermediate | 3-(2-(difluoromethoxy)-6-methylpyridin-3- | |
| | amine 128 | yl)-1-((1s,4s)-4-(2-hydroxypropan-2- | |
| | | yl)cyclohexyl)-1-(2-isopropylphenyl)urea | |
| 239 | 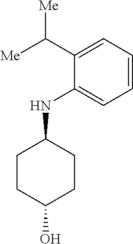 [SMILES: CC(C)c1ccccc1N[C@H]1CC[C@H](O)CC1] | 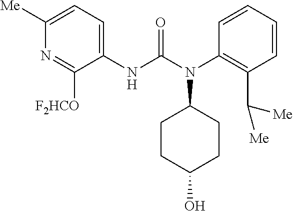 [SMILES: Cc1ccc(NC(=O)N(c2ccccc2C(C)C)[C@H]2CC[C@H](O)CC2)c(OC(F)F)n1] | 433.8 [M + H]⁺ |
| | Intermediate | 3-(2-(difluoromethoxy)-6-methylpyridin-3- | |
| | amine 129 | yl)-1-((1r,4r)-4-hydroxycyclohexyl)-1-(2- | |
| | | isopropylphenyl)urea | |
| 240 | 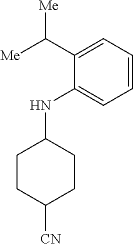 [SMILES: CC(C)c1ccccc1NC1CCC(C#N)CC1] | 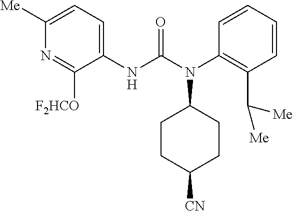 [SMILES: Cc1ccc(NC(=O)N(c2ccccc2C(C)C)[C@H]2CC[C@@H](C#N)CC2)c(OC(F)F)n1] | 442.8 [M + H]⁺ |
| | Intermediate | 1-((1s,4s)-4-cyanocyclohexyl)-3-(2- | |
| | amine 130 | (difluoromethoxy)-6-methylpyridin-3-yl)-1- | |
| | | (2-isopropylphenyl)urea | |
| 241 | 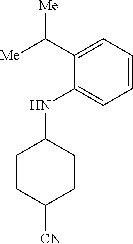 [SMILES: CC(C)c1ccccc1NC1CCC(C#N)CC1] | 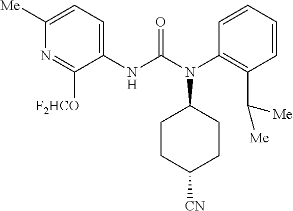 [SMILES: Cc1ccc(NC(=O)N(c2ccccc2C(C)C)[C@H]2CC[C@H](C#N)CC2)c(OC(F)F)n1] | 442.8 [M + H]⁺ |
| | Intermediate | 1-((1r,4r)-4-cyanocyclohexyl)-3-(2- | |
| | amine 130 | (difluoromethoxy)-6-methylpyridin-3-yl)-1- | |
| | | (2-isopropylphenyl)urea | |
| 250 | 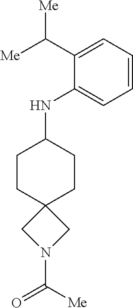 [SMILES: CC(=O)N1CC2(CCC(Nc3ccccc3C(C)C)CC2)C1] | 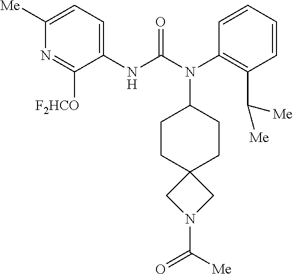 [SMILES: CC(=O)N1CC2(CCC(N(C(=O)Nc3ccc(C)nc3OC(F)F)c3ccccc3C(C)C)CC2)C1] | 500.9 [M + H]⁺ |
| | Intermediate | 1-(2-acetyl-2-azaspiro[3.5]nonan-7-yl)-3-(2- | |
| | amine 133 | (difluoromethoxy)-6-methylpyridin-3-yl)-1- | |
| | | (2-isopropylphenyl)urea | |
| 251 | 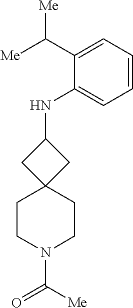 [SMILES: CC(=O)N1CCC2(CC1)CC(Nc1ccccc1C(C)C)C2] | 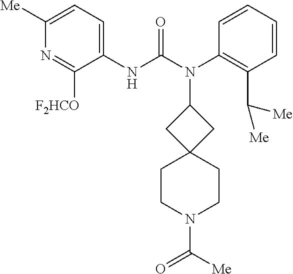 [SMILES: CC(=O)N1CCC2(CC1)CC(N(C(=O)Nc1ccc(C)nc1OC(F)F)c1ccccc1C(C)C)C2] | 500.9 [M + H]⁺ |
| | Intermediate | 1-(7-acetyl-7-azaspiro[3.5]nonan-2-yl)-3-(2- | |
| | amine 135 | (difluoromethoxy)-6-methylpyridin-3-yl)-1- | |
| | | (2-isopropylphenyl)urea | |
| 255 | 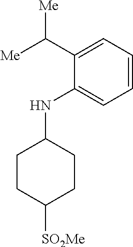 [SMILES: CC(C)c1ccccc1NC1CCC(S(C)(=O)=O)CC1] | 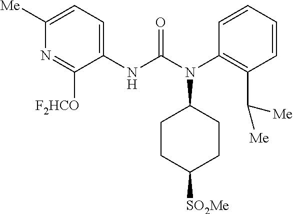 [SMILES: Cc1ccc(NC(=O)N(c2ccccc2C(C)C)[C@H]2CC[C@@H](S(C)(=O)=O)CC2)c(OC(F)F)n1] | 495.8 [M + H]⁺ |
| | Intermediate | 3-(2-(difluoromethoxy)-6-methylpyridin-3- | |
| | amine 138 | yl)-1-(2-isopropylphenyl)-1-((1s,4s)-4- | |
| | | (methylsulfonyl)cyclohexyl)urea | |
| 256 | 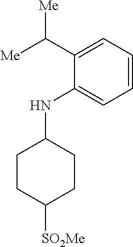 [SMILES: CC(C)c1ccccc1NC1CCC(S(C)(=O)=O)CC1] | 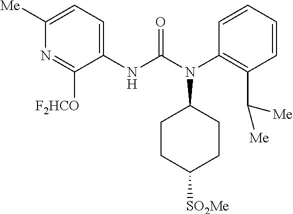 [SMILES: Cc1ccc(NC(=O)N(c2ccccc2C(C)C)[C@H]2CC[C@H](S(C)(=O)=O)CC2)c(OC(F)F)n1] | 495.8 [M + H]⁺ |
| | Intermediate | 3-(2-(difluoromethoxy)-6-methylpyridin-3- | |
| | amine 138 | yl)-1-(2-isopropylphenyl)-1-((1r,4r)-4- | |
| | | (methylsulfonyl)cyclohexyl)urea | |
| 260 | 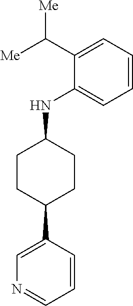 [SMILES: CC(C)c1ccccc1N[C@H]1CC[C@@H](c2cccnc2)CC1] | 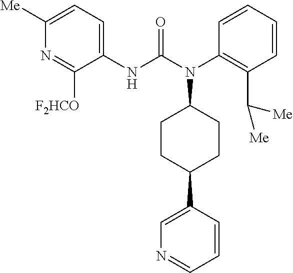 [SMILES: Cc1ccc(NC(=O)N(c2ccccc2C(C)C)[C@H]2CC[C@@H](c3cccnc3)CC2)c(OC(F)F)n1] | 494.9 [M + H]⁺ |
| | Intermediate | 3-(2-(difluoromethoxy)-6-methylpyridin-3- | |
| | amine 140 | yl)-1-(2-isopropylphenyl)-1-((1s,4s)-4- | |
| | | (pyridin-3-yl)cyclohexyl)urea | |
| 261 | 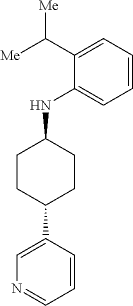 [SMILES: CC(C)c1ccccc1N[C@H]1CC[C@H](c2cccnc2)CC1] | 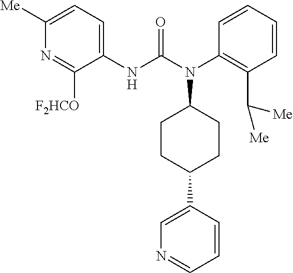 [SMILES: Cc1ccc(NC(=O)N(c2ccccc2C(C)C)[C@H]2CC[C@H](c3cccnc3)CC2)c(OC(F)F)n1] | 494.9 [M + H]⁺ |
| | Intermediate | 3-(2-(difluoromethoxy)-6-methylpyridin-3- | |
| | amine 141 | yl)-1-(2-isopropylphenyl)-1-((1r,4r)-4- | |
| | | (pyridin-3-yl)cyclohexyl)urea | |
| 263 | 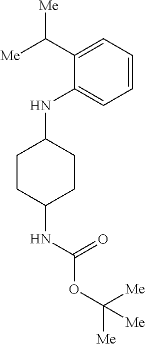 [SMILES: CC(C)c1ccccc1NC1CCC(NC(=O)OC(C)(C)C)CC1] | 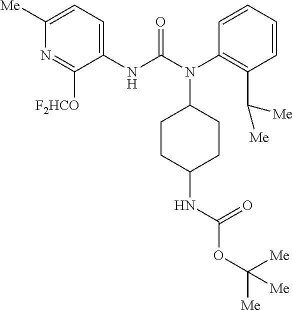 [SMILES: Cc1ccc(NC(=O)N(c2ccccc2C(C)C)C2CCC(NC(=O)OC(C)(C)C)CC2)c(OC(F)F)n1] | 554.9 [M + Na]⁺ |
| | Intermediate | tert-butyl (4-(3-(2-(difluoromethoxy)-6- | |
| | amine 144 | methylpyridin-3-yl)-1-(2- | |
| | | isopropylphenyl)ureido)cyclohexyl)carbamate | |
| 270 | 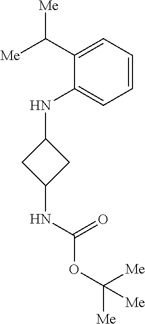 [SMILES: CC(C)c1ccccc1NC1CC(NC(=O)OC(C)(C)C)C1] | 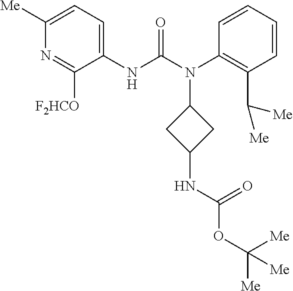 [SMILES: Cc1ccc(NC(=O)N(c2ccccc2C(C)C)C2CC(NC(=O)OC(C)(C)C)C2)c(OC(F)F)n1] | 526.8 [M + Na]⁺ |
| | Intermediate | tert-butyl (3-(3-(2-(difluoromethoxy)-6- | |
| | amine 145 | methylpyridin-3-yl)-1-(2- | |
| | | isopropylphenyl)ureido)cyclobutyl)carbamate | |

The following examples were prepared in an analogous fashion to Example 1, but substituting 2-(difluoromethoxy)-6-methylpyridin-3-amine with the requisite Starting Amine 1, and Intermediate amine 5 with the requisite Starting Amine 2. In Example 41, Example 42, Example 43, Example 44, Example 45, Example 46, Example 47, Example 48, Example 49, Example 50, Example 51, Example 52, Example 53, Example 54, Example 55, Example 56, Example 57, Example 58, Example 59, Example 60, Example 61, Example 62, Example 310, Example 311, Example 312, Example 313, and Example 314, phosgene (1.5 equiv) was also substituted with triphosgene (0.5 equiv, Sigma-Aldrich). In Example 53, Example 61, Example 310, and Example 313, the reaction was heated at 40° C. for an additional 2 days. In Example 46, Example 47, Example 48, Example 49, Example 56, Example 57, Example 58, Example 59, Example 60, and Example 312, the reaction was heated at 40° C. for an additional 7 days.

| | Starting Amine | Starting Amine | | LCMS |
|---|---|---|---|---|
| Example | 1 | 2 | Structure | (m/z) |
| 37 | 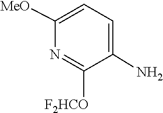 [SMILES: COc1ccc(N)c(OC(F)F)n1] | 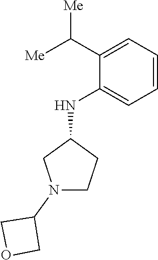 [SMILES: CC(C)c1ccccc1N[C@@H]1CCN(C2COC2)C1] | 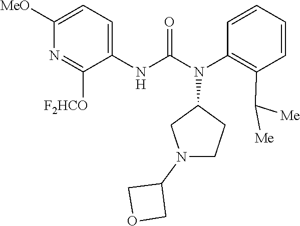 [SMILES: COc1ccc(NC(=O)N(c2ccccc2C(C)C)[C@@H]2CCN(C3COC3)C2)c(OC(F)F)n1] | 477.1 [M + H]⁺ |
| | Intermediate | Intermediate | (R)-3-(2-(difluoromethoxy)-6- | |
| | amine 95 | amine 25 | methoxypyridin-3-yl)-1-(2- | |
| | | | isopropylphenyl)-1-(1- | |
| | | | (oxetan-3-yl)pyrrolidin-3- | |
| | | | yl)urea | |
| 38 | 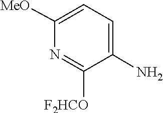 [SMILES: COc1ccc(N)c(OC(F)F)n1] | 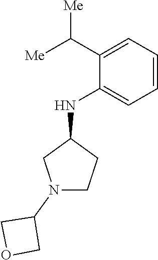 [SMILES: CC(C)c1ccccc1N[C@H]1CCN(C2COC2)C1] | 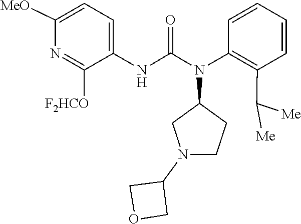 [SMILES: COc1ccc(NC(=O)N(c2ccccc2C(C)C)[C@H]2CCN(C3COC3)C2)c(OC(F)F)n1] | 477.1 [M + H]⁺ |
| | Intermediate | Intermediate | (S)-3-(2-(difluoromethoxy)-6- | |
| | amine 95 | amine 26 | methoxypyridin-3-yl)-1-(2- | |
| | | | isopropylphenyl)-1-(1- | |
| | | | (oxetan-3-yl)pyrrolidin-3- | |
| | | | yl)urea | |
| 39 | 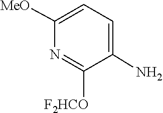 [SMILES: COc1ccc(N)c(OC(F)F)n1] | 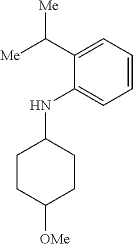 [SMILES: COC1CCC(Nc2ccccc2C(C)C)CC1] | 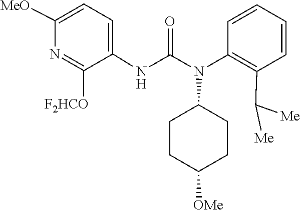 [SMILES: COc1ccc(NC(=O)N(c2ccccc2C(C)C)[C@H]2CC[C@@H](OC)CC2)c(OC(F)F)n1] | 464.1 [M + H]⁺ |
| | Intermediate | Intermediate | 3-(2-(difluoromethoxy)-6- | |
| | amine 95 | amine 72 | methoxypyridin-3-yl)-1-(2- | |
| | | | isopropylphenyl)-1-((1s,4s)-4- | |
| | | | methoxycyclohexyl)urea | |
| 40 | 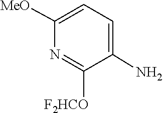 [SMILES: COc1ccc(N)c(OC(F)F)n1] | 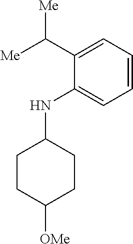 [SMILES: COC1CCC(Nc2ccccc2C(C)C)CC1] | 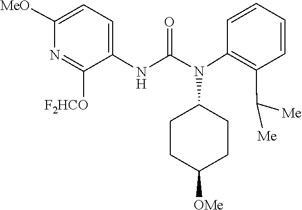 [SMILES: COc1ccc(NC(=O)N(c2ccccc2C(C)C)[C@H]2CC[C@H](OC)CC2)c(OC(F)F)n1] | 464.1 [M + H]⁺ |
| | Intermediate | Intermediate | 3-(2-(difluoromethoxy)-6- | |
| | amine 95 | amine 72 | methoxypyridin-3-yl)-1-(2- | |
| | | | isopropylphenyl)-1-((1r,4r)-4- | |
| | | | methoxycyclohexyl)urea | |
| 41 | 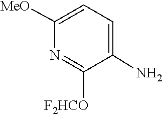 [SMILES: COc1ccc(N)c(OC(F)F)n1] | 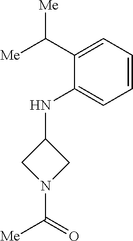 [SMILES: CC(=O)N1CC(Nc2ccccc2C(C)C)C1] | 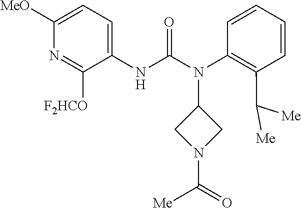 [SMILES: COc1ccc(NC(=O)N(c2ccccc2C(C)C)C2CN(C(C)=O)C2)c(OC(F)F)n1] | 449.2 [M + H]⁺ |
| | Intermediate | Intermediate | 1-(1-acetylazetidin-3-yl)-3-(2- | |
| | amine 95 | amine 79 | (difluoromethoxy)-6- | |
| | | | methoxypyridin-3-yl)-1-(2- | |
| | | | isopropylphenyl)urea | |
| 42 | 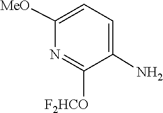 [SMILES: COc1ccc(N)c(OC(F)F)n1] | 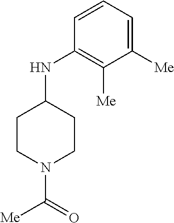 [SMILES: CC(=O)N1CCC(Nc2cccc(C)c2C)CC1] | 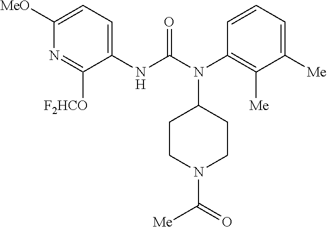 [SMILES: COc1ccc(NC(=O)N(c2cccc(C)c2C)C2CCN(C(C)=O)CC2)c(OC(F)F)n1] | 463.2 [M + H]⁺ |
| | Intermediate | Intermediate | 1-(1-acetylpiperidin-4-yl)-3- | |
| | amine 95 | amine 55 | (2-(difluoromethoxy)-6- | |
| | | | methoxypyridin-3-yl)-1-(2,3- | |
| | | | dimethylphenyl)urea | |
| 43 | 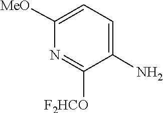 [SMILES: COc1ccc(N)c(OC(F)F)n1] | 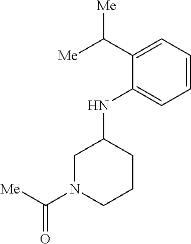 [SMILES: CC(=O)N1CCCC(Nc2ccccc2C(C)C)C1] | 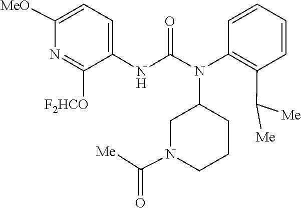 [SMILES: COc1ccc(NC(=O)N(c2ccccc2C(C)C)C2CCCN(C(C)=O)C2)c(OC(F)F)n1] | 477.3 [M + H]⁺ |
| | Intermediate | Intermediate | 1-(1-acetylpiperidin-3-yl)-3- | |
| | amine 95 | amine 32 | (2-(difluoromethoxy)-6- | |
| | | | methoxypyridin-3-yl)-1-(2- | |
| | | | isopropylphenyl)urea | |
| 44 | 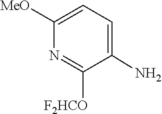 [SMILES: COc1ccc(N)c(OC(F)F)n1] | 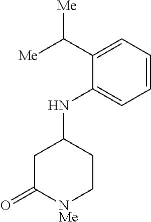 [SMILES: CC(C)c1ccccc1NC1CCN(C)C(=O)C1] | 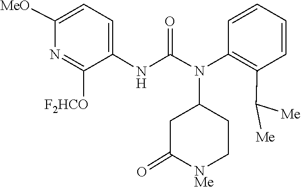 [SMILES: COc1ccc(NC(=O)N(c2ccccc2C(C)C)C2CCN(C)C(=O)C2)c(OC(F)F)n1] | 463.2 [M + H]⁺ |
| | Intermediate | Intermediate | 3-(2-(difluoromethoxy)-6- | |
| | amine 95 | amine 33 | methoxypyridin-3-yl)-1-(2- | |
| | | | isopropylphenyl)-1-(1- | |
| | | | methyl-2-oxopiperidin-4- | |
| | | | yl)urea | |
| 45 | 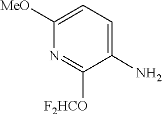 [SMILES: COc1ccc(N)c(OC(F)F)n1] | 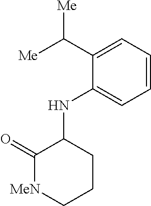 [SMILES: CC(C)c1ccccc1NC1CCCN(C)C1=O] | 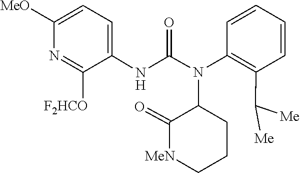 [SMILES: COc1ccc(NC(=O)N(c2ccccc2C(C)C)C2CCCN(C)C2=O)c(OC(F)F)n1] | 463.2 [M + H]⁺ |
| | Intermediate | Intermediate | 3-(2-(difluoromethoxy)-6- | |
| | amine 95 | amine 34 | methoxypyridin-3-yl)-1-(2- | |
| | | | isopropylphenyl)-1-(1- | |
| | | | methyl-2-oxopiperidin-3- | |
| | | | yl)urea | |
| 46 | 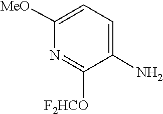 [SMILES: COc1ccc(N)c(OC(F)F)n1] | 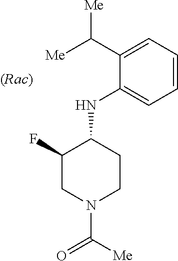 [SMILES: CC(=O)N1CC[C@@H](Nc2ccccc2C(C)C)[C@H](F)C1] | 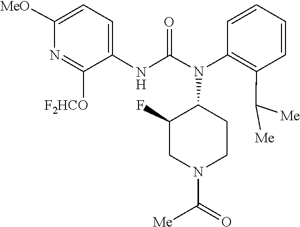 [SMILES: COc1ccc(NC(=O)N(c2ccccc2C(C)C)[C@@H]2CCN(C(C)=O)C[C@H]2F)c(OC(F)F)n1] | 495.3 [M + H]⁺ |
| | Intermediate | Intermediate | rac-1-((3R,4R)-1-acetyl-3- | |
| | amine 95 | amine 81 | fluoropiperidin-4-yl)-3-(2- | |
| | | | (difluoromethoxy)-6- | |
| | | | methoxypyridin-3-yl)-1-(2- | |
| | | | isopropylphenyl)urea | |
| 47 | 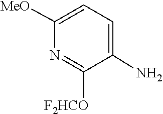 [SMILES: COc1ccc(N)c(OC(F)F)n1] | 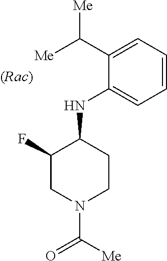 [SMILES: CC(=O)N1CC[C@H](Nc2ccccc2C(C)C)[C@H](F)C1] | 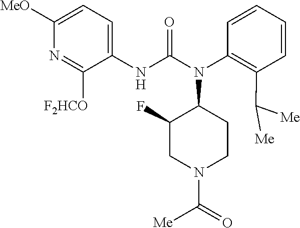 [SMILES: COc1ccc(NC(=O)N(c2ccccc2C(C)C)[C@H]2CCN(C(C)=O)C[C@H]2F)c(OC(F)F)n1] | 495.3 [M + H]⁺ |
| | Intermediate | Intermediate | rac-1-((3R,4S)-1-acetyl-3- | |
| | amine 95 | amine 82 | fluoropiperidin-4-yl)-3-(2- | |
| | | | (difluoromethoxy)-6- | |
| | | | methoxypyridin-3-yl)-1-(2- | |
| | | | isopropylphenyl)urea | |
| 48 | 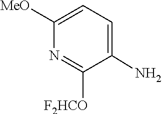 [SMILES: COc1ccc(N)c(OC(F)F)n1] | 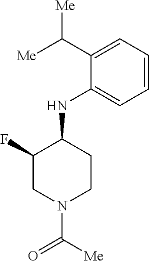 [SMILES: CC(=O)N1CC[C@H](Nc2ccccc2C(C)C)[C@H](F)C1] | 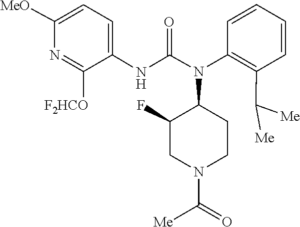 [SMILES: COc1ccc(NC(=O)N(c2ccccc2C(C)C)[C@H]2CCN(C(C)=O)C[C@H]2F)c(OC(F)F)n1] | 495.3 [M + H]⁺ |
| | Intermediate | Intermediate | 1-((3R,4S)-1-acetyl-3- | |
| | amine 95 | amine 83 | fluoropiperidin-4-yl)-3-(2- | |
| | | | (difluoromethoxy)-6- | |
| | | | methoxypyridin-3-yl)-1-(2- | |
| | | | isopropylphenyl)urea | |
| 49 | 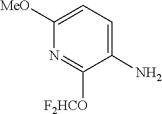 [SMILES: COc1ccc(N)c(OC(F)F)n1] | 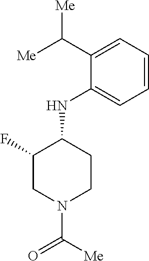 [SMILES: CC(=O)N1CC[C@@H](Nc2ccccc2C(C)C)[C@@H](F)C1] | 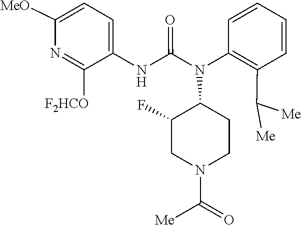 [SMILES: COc1ccc(NC(=O)N(c2ccccc2C(C)C)[C@@H]2CCN(C(C)=O)C[C@@H]2F)c(OC(F)F)n1] | 495.2 [M + H]⁺ |
| | Intermediate | Intermediate | 1-((3S,4R)-1-acetyl-3- | |
| | amine 95 | amine 84 | fluoropiperidin-4-yl)-3-(2- | |
| | | | (difluoromethoxy)-6- | |
| | | | methoxypyridin-3-yl)-1-(2- | |
| | | | isopropylphenyl)urea | |
| 50 | 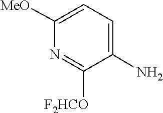 [SMILES: COc1ccc(N)c(OC(F)F)n1] | 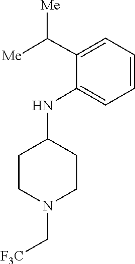 [SMILES: CC(C)c1ccccc1NC1CCN(CC(F)(F)F)CC1] | 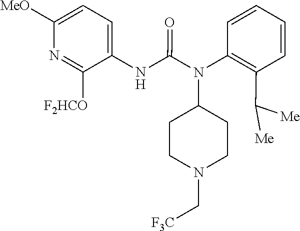 [SMILES: COc1ccc(NC(=O)N(c2ccccc2C(C)C)C2CCN(CC(F)(F)F)CC2)c(OC(F)F)n1] | 517.2 [M + H]⁺ |
| | Intermediate | Intermediate | 3-(2-(difluoromethoxy)-6- | |
| | amine 95 | amine 39 | methoxypyridin-3-yl)-1-(2- | |
| | | | isopropylphenyl)-1-(1-(2,2,2- | |
| | | | trifluoroethyl)piperidin-4- | |
| | | | yl)urea | |
| 51 | 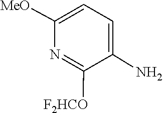 [SMILES: COc1ccc(N)c(OC(F)F)n1] | 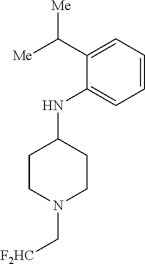 [SMILES: CC(C)c1ccccc1NC1CCN(CC(F)F)CC1] | 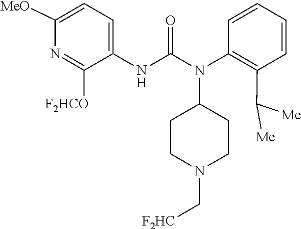 [SMILES: COc1ccc(NC(=O)N(c2ccccc2C(C)C)C2CCN(CC(F)F)CC2)c(OC(F)F)n1] | 499.3 [M + H]⁺ |
| | Intermediate | Intermediate | 1-(1-(2,2- | |
| | amine 95 | amine 40 | difluoroethyl)piperidin-4-yl)- | |
| | | | 3-(2-(difluoromethoxy)-6- | |
| | | | methoxypyridin-3-yl)-1-(2- | |
| | | | isopropylphenyl)urea | |
| 52 | 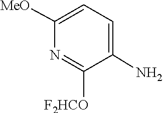 [SMILES: COc1ccc(N)c(OC(F)F)n1] | 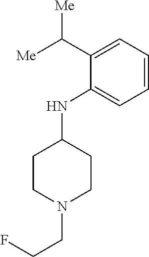 [SMILES: CC(C)c1ccccc1NC1CCN(CCF)CC1] | 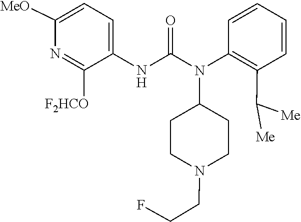 [SMILES: COc1ccc(NC(=O)N(c2ccccc2C(C)C)C2CCN(CCF)CC2)c(OC(F)F)n1] | 481.3 [M + H]⁺ |
| | Intermediate | Intermediate | 3-(2-(difluoromethoxy)-6- | |
| | amine 95 | amine 41 | methoxypyridin-3-yl)-1-(1-(2- | |
| | | | fluoroethyl)piperidin-4-yl)-1- | |
| | | | (2-isopropylphenyl)urea | |
| 53 | 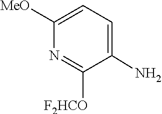 [SMILES: COc1ccc(N)c(OC(F)F)n1] | 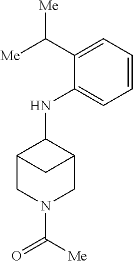 [SMILES: CC(=O)N1CC2CC(C1)C2Nc1ccccc1C(C)C] | 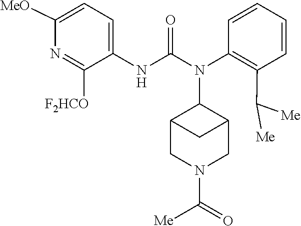 [SMILES: COc1ccc(NC(=O)N(c2ccccc2C(C)C)C2C3CC2CN(C(C)=O)C3)c(OC(F)F)n1] | 489.3 [M + H]⁺ |
| | Intermediate | Intermediate | 1-(3-acetyl-3- | |
| | amine 95 | amine 85 | azabicyclo[3.1.1]heptan-6-yl)- | |
| | | | 3-(2-(difluoromethoxy)-6- | |
| | | | methoxypyridin-3-yl)-1-(2- | |
| | | | isopropylphenyl)urea | |
| 54 | 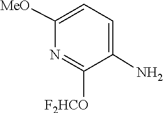 [SMILES: COc1ccc(N)c(OC(F)F)n1] | 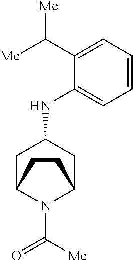 | 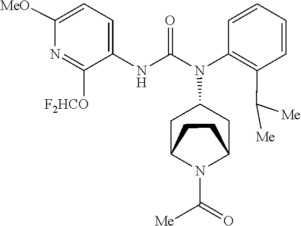 | 503.3 [M + H]⁺ |
| | Intermediate | Intermediate | 1-((1R,3s,5S)-8-acetyl-8- | |
| | amine 95 | amine 86 | azabicyclo[3.2.1]octan-3-yl)- | |
| | | | 3-(2-(difluoromethoxy)-6- | |
| | | | methoxypyridin-3-yl)-1-(2- | |
| | | | isopropylphenyl)urea | |
| 55 | 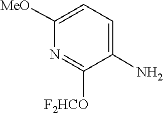 [SMILES: COc1ccc(N)c(OC(F)F)n1] | 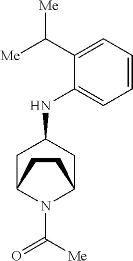 | 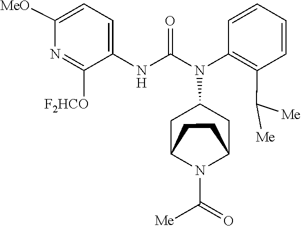 | 503.3 [M + H]⁺ |
| | Intermediate | Intermediate | 1-((1R,3r,5S)-8-acetyl-8- | |
| | amine 95 | amine 87 | azabicyclo[3.2.1]octan-3-yl)- | |
| | | | 3-(2-(difluoromethoxy)-6- | |
| | | | methoxypyridin-3-yl)-1-(2- | |
| | | | isopropylphenyl)urea | |
| 56 | 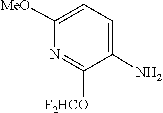 [SMILES: COc1ccc(N)c(OC(F)F)n1] | 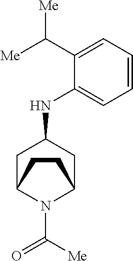 | 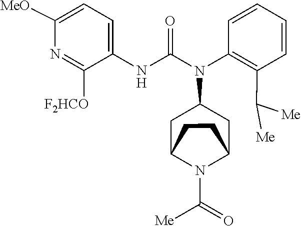 | 503.3 [M + H]⁺ |
| | Intermediate | Intermediate | 1-((1R,5S,8s)-3-acetyl-3- | |
| | amine 95 | amine 88 | azabicyclo[3.2.1]octan-8-yl)- | |
| | | | 3-(2-(difluoromethoxy)-6- | |
| | | | methoxypyridin-3-yl)-1-(2- | |
| | | | isopropylphenyl)urea | |
| 57 | 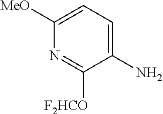 [SMILES: COc1ccc(N)c(OC(F)F)n1] | 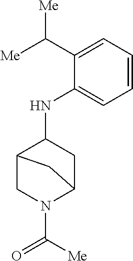 [SMILES: CC(=O)N1CC2CC1CC2Nc1ccccc1C(C)C] | 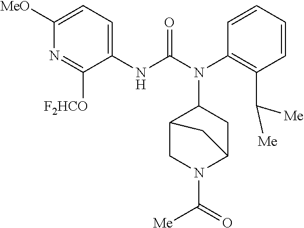 [SMILES: COc1ccc(NC(=O)N(c2ccccc2C(C)C)C2CC3CC2CN3C(C)=O)c(OC(F)F)n1] | 489.3 [M + H]⁺ |
| | Intermediate | Intermediate | 1-(2-acetyl-2- | |
| | amine 95 | amine 89 | azabicyclo[2.2.1]heptan-5-yl)- | |
| | | | 3-(2-(difluoromethoxy)-6- | |
| | | | methoxypyridin-3-yl)-1-(2- | |
| | | | isopropylphenyl)urea | |
| 58 | 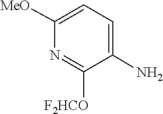 [SMILES: COc1ccc(N)c(OC(F)F)n1] | 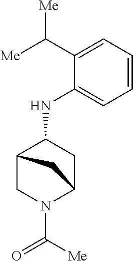 [SMILES: CC(=O)N1C[C@H]2C[C@@H]1C[C@H]2Nc1ccccc1C(C)C] | 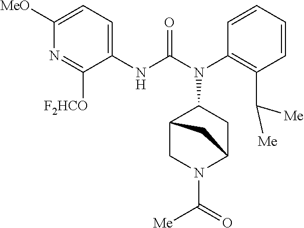 [SMILES: COc1ccc(NC(=O)N(c2ccccc2C(C)C)[C@@H]2C[C@H]3C[C@@H]2CN3C(C)=O)c(OC(F)F)n1] | 489.2 [M + H]⁺ |
| | Intermediate | Intermediate | 1-((1R,4R,5R)-2-acetyl-2- | |
| | amine 95 | amine 90 | azabicyclo[2.2.1]heptan-5-yl)- | |
| | | | 3-(2-(difluoromethoxy)-6- | |
| | | | methoxypyridin-3-yl)-1-(2- | |
| | | | isopropylphenyl)urea | |
| 59 | 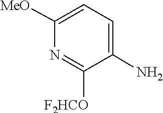 [SMILES: COc1ccc(N)c(OC(F)F)n1] | 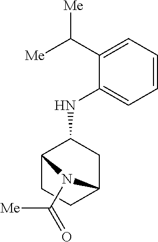 [SMILES: CC(=O)N1[C@H]2CC[C@@H]1[C@H](Nc1ccccc1C(C)C)C2] | 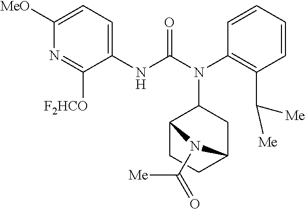 [SMILES: COc1ccc(NC(=O)N(c2ccccc2C(C)C)C2C[C@@H]3CC[C@H]2N3C(C)=O)c(OC(F)F)n1] | 489.2 [M + H]⁺ |
| | Intermediate | Intermediate | rac-1-((1R,2R,4S)-7-acetyl-7- | |
| | amine 95 | amine 91 | azabicyclo[2.2.1]heptan-2-yl)- | |
| | | | 3-(2-(difluoromethoxy)-6- | |
| | | | methoxypyridin-3-yl)-1-(2- | |
| | | | isopropylphenyl)urea | |
| 60 | 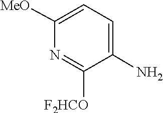 [SMILES: COc1ccc(N)c(OC(F)F)n1] | 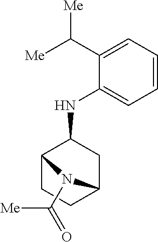 [SMILES: CC(=O)N1[C@H]2CC[C@@H]1[C@@H](Nc1ccccc1C(C)C)C2] | 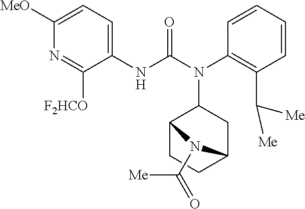 [SMILES: COc1ccc(NC(=O)N(c2ccccc2C(C)C)C2C[C@@H]3CC[C@H]2N3C(C)=O)c(OC(F)F)n1] | 489.2 [M + H]⁺ |
| | Intermediate | Intermediate | rac-1-((1R,2S,4S)-7-acetyl-7- | |
| | amine 95 | amine 92 | azabicyclo[2.2.1]heptan-2-yl)- | |
| | | | 3-(2-(difluoromethoxy)-6- | |
| | | | methoxypyridin-3-yl)-1-(2- | |
| | | | isopropylphenyl)urea | |
| 61 | 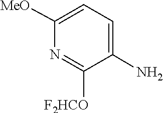 [SMILES: COc1ccc(N)c(OC(F)F)n1] | 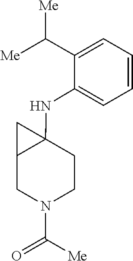 [SMILES: CC(=O)N1CCC2(Nc3ccccc3C(C)C)CC2C1] | 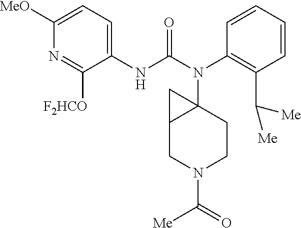 [SMILES: COc1ccc(NC(=O)N(c2ccccc2C(C)C)C23CCN(C(C)=O)CC2C3)c(OC(F)F)n1] | 489.3 [M + H]⁺ |
| | Intermediate | Intermediate | 1-(3-acetyl-3- | |
| | amine 95 | amine 93 | azabicyclo[4.1.0]heptan-6-yl)- | |
| | | | 3-(2-(difluoromethoxy)-6- | |
| | | | methoxypyridin-3-yl)-1-(2- | |
| | | | isopropylphenyl)urea | |
| 62 | 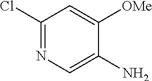 | 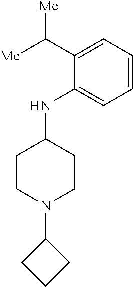 [SMILES: CC(C)c1ccccc1NC1CCN(C2CCC2)CC1] | 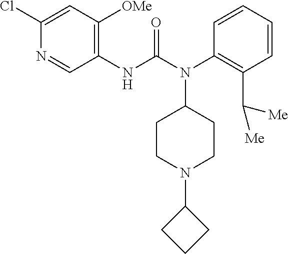 [SMILES: COc1cc(Cl)ncc1NC(=O)N(c1ccccc1C(C)C)C1CCN(C2CCC2)CC1] | 457.2, 459.2 [M + H]⁺ |
| | Combi-Blocks | Intermediate | 3-(6-chloro-4- | |
| | | amine 28 | methoxypyridin-3-yl)-1-(1- | |
| | | | cyclobutylpiperidin-4-yl)-1- | |
| | | | (2-isopropylphenyl)urea | |
| 257 | 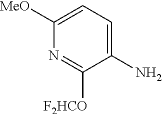 [SMILES: COc1ccc(N)c(OC(F)F)n1] | 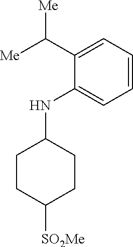 [SMILES: CC(C)c1ccccc1NC1CCC(S(C)(=O)=O)CC1] | 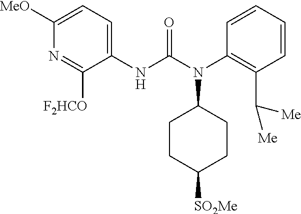 [SMILES: COc1ccc(NC(=O)N(c2ccccc2C(C)C)[C@H]2CC[C@@H](S(C)(=O)=O)CC2)c(OC(F)F)n1] | 512.1 [M + H]⁺ |
| | Intermediate | Intermediate | 3-(2-(difluoromethoxy)-6- | |
| | amine 95 | amine 138 | methoxypyridin-3-yl)-1-(2- | |
| | | | isopropylphenyl)-1-((1s,4s)-4- | |
| | | | (methylsulfonyl)cyclohexyl) | |
| | | | urea | |
| 258 | 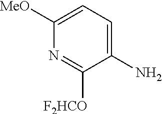 [SMILES: COc1ccc(N)c(OC(F)F)n1] | 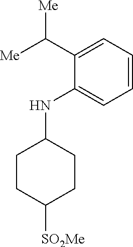 [SMILES: CC(C)c1ccccc1NC1CCC(S(C)(=O)=O)CC1] | 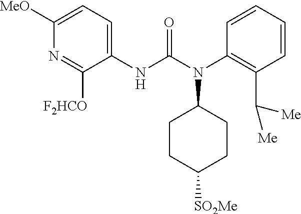 [SMILES: COc1ccc(NC(=O)N(c2ccccc2C(C)C)[C@H]2CC[C@H](S(C)(=O)=O)CC2)c(OC(F)F)n1] | 512.1 [M + H]⁺ |
| | Intermediate | Intermediate | 3-(2-(difluoromethoxy)-6- | |
| | amine 95 | amine 138 | methoxypyridin-3-yl)-1-(2- | |
| | | | isopropylphenyl)-1-((1r,4r)-4- | |
| | | | (methylsulfonyl)cyclohexyl) | |
| | | | urea | |
| 310 | 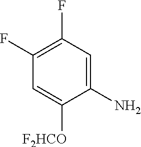 [SMILES: Nc1cc(F)c(F)cc1OC(F)F] | 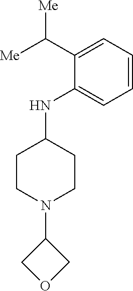 [SMILES: CC(C)c1ccccc1NC1CCN(C2COC2)CC1] | 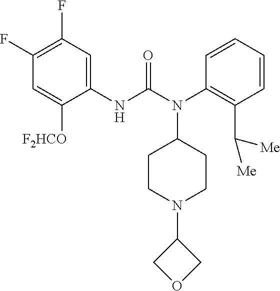 [SMILES: CC(C)c1ccccc1N(C(=O)Nc1cc(F)c(F)cc1OC(F)F)C1CCN(C2COC2)CC1] | 496.2 [M + H]⁺ |
| | Intermediate | Intermediate | 3-(2-(difluoromethoxy)-4,5- | |
| | amine 101 | amine 162 | difluorophenyl)-1-(2- | |
| | | | isopropylphenyl)-1-(1- | |
| | | | (oxetan-3-yl)piperidin-4- | |
| | | | yl)urea | |
| 311 | 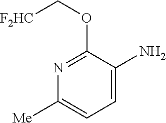 [SMILES: Cc1ccc(N)c(OCC(F)F)n1] | 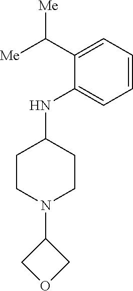 [SMILES: CC(C)c1ccccc1NC1CCN(C2COC2)CC1] | 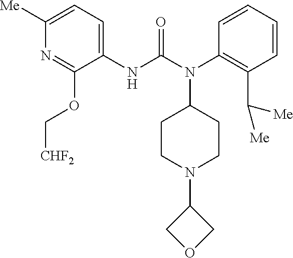 [SMILES: Cc1ccc(NC(=O)N(c2ccccc2C(C)C)C2CCN(C3COC3)CC2)c(OCC(F)F)n1] | 489.2 [M + H]⁺ |
| | Intermediate | Intermediate | 3-(2-(2,2-difluoroethoxy)-6- | |
| | amine 163 | amine 162 | methylpyridin-3-yl)-1-(2- | |
| | | | isopropylphenyl)-1-(1- | |
| | | | (oxetan-3-yl)piperidin-4- | |
| | | | yl)urea | |
| 312 | 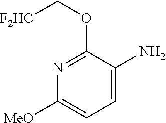 [SMILES: COc1ccc(N)c(OCC(F)F)n1] | 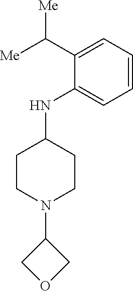 [SMILES: CC(C)c1ccccc1NC1CCN(C2COC2)CC1] | 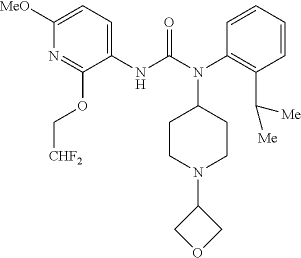 [SMILES: COc1ccc(NC(=O)N(c2ccccc2C(C)C)C2CCN(C3COC3)CC2)c(OCC(F)F)n1] | 505.3 [M + H]⁺ |
| | Intermediate | Intermediate | 3-(2-(2,2-difluoroethoxy)-6- | |
| | amine 107 | amine 162 | methoxypyridin-3-yl)-1-(2- | |
| | | | isopropylphenyl)-1-(1- | |
| | | | (oxetan-3-yl)piperidin-4- | |
| | | | yl)urea | |
| 313 | 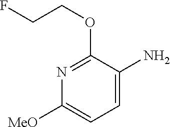 [SMILES: COc1ccc(N)c(OCCF)n1] | 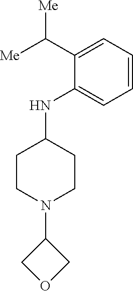 [SMILES: CC(C)c1ccccc1NC1CCN(C2COC2)CC1] | 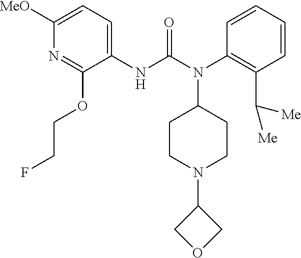 [SMILES: COc1ccc(NC(=O)N(c2ccccc2C(C)C)C2CCN(C3COC3)CC2)c(OCCF)n1] | 487.3 [M + H]⁺ |
| | Intermediate | Intermediate | 3-(2-(2-fluoroethoxy)-6- | |
| | amine 164 | amine 162 | methoxypyridin-3-yl)-1-(2- | |
| | | | isopropylphenyl)-1-(1- | |
| | | | (oxetan-3-yl)piperidin-4- | |
| | | | yl)urea | |
| 314 | 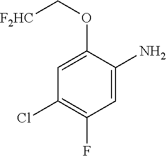 [SMILES: Nc1cc(F)c(Cl)cc1OCC(F)F] | 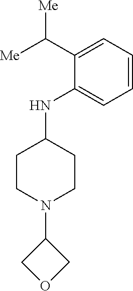 [SMILES: CC(C)c1ccccc1NC1CCN(C2COC2)CC1] | 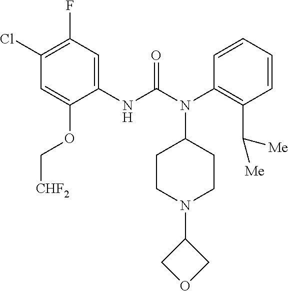 [SMILES: CC(C)c1ccccc1N(C(=O)Nc1cc(F)c(Cl)cc1OCC(F)F)C1CCN(C2COC2)CC1] | 526.2 [M + H]⁺ |
| | Intermediate | Intermediate | 3-(4-chloro-2-(2,2- | |
| | amine 165 | amine 162 | difluoroethoxy)-5- | |
| | | | fluorophenyl)-1-(2- | |
| | | | isopropylphenyl)-1-(1- | |
| | | | (oxetan-3-yl)piperidin-4- | |
| | | | yl)urea | |

### Example 63: Preparation of methyl 4-(3-(2-(difluoromethoxy)-6-methoxypyridin-3-yl)-1-(2-isopropylphenyl)ureido)piperidine-1-carboxylate

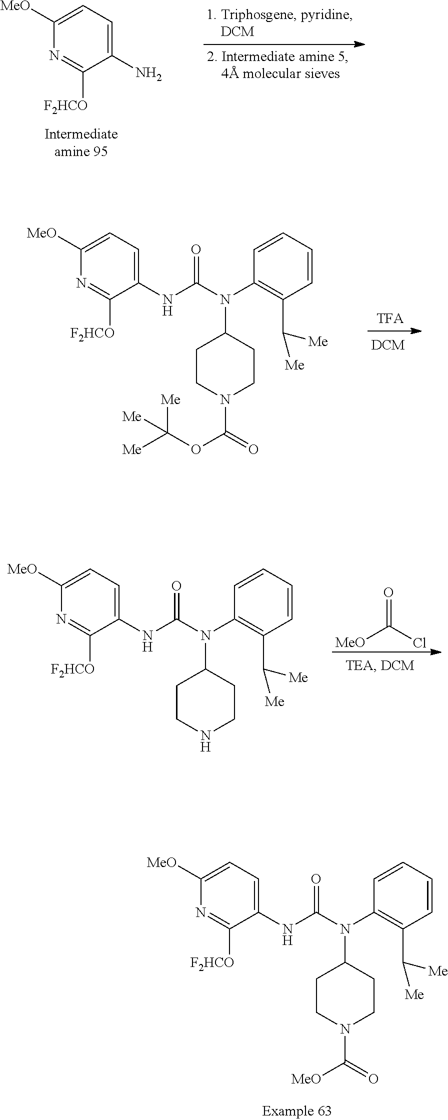 [SMILES: COc1ccc(N)c(OC(F)F)n1 ; COc1ccc(NC(=O)N(c2ccccc2C(C)C)C2CCN(C(=O)OC(C)(C)C)CC2)c(OC(F)F)n1 ; COc1ccc(NC(=O)N(c2ccccc2C(C)C)C2CCNCC2)c(OC(F)F)n1 ; COC(=O)Cl ; COC(=O)N1CCC(N(C(=O)Nc2ccc(OC)nc2OC(F)F)c2ccccc2C(C)C)CC1]

Step 1: In a dried, round-bottom flask equipped with a magnetic stirrer was combined Intermediate amine 95 (1 equiv) and pyridine (3 equiv, Sigma-Aldrich) in anhydrous dichloromethane (0.41 M). To this was then added triphosgene (0.5 equiv, Sigma-Aldrich) in one rapid portion at RT and the resulting solution was stirred at RT for 30 min. To this mixture was then added, at 0° C., Intermediate amine 5 (1 equiv) and freshly activated 4 Å molecular sieves. The resulting suspension was warmed to and stirred at RT for 16 h, before the reaction was quenched with water. The aqueous layer was separated and back extracted with DCM. The combined organic extracts were dried over Na₂SO₄, filtered, and the filtrate concentrated in vacuo. Purification of the crude product thus obtained by way of reverse-phase column chromatography (C₁₈, gradient elution: 95:5 (v/v) H₂O: MeCN→MeCN) afforded tert-butyl 4-(3-(2-(difluoromethoxy)-6-methoxypyridin-3-yl)-1-(2-isopropylphenyl)ureido)piperidine-1-carboxylate as a yellow solid (37% yield).

Step 2: In a round-bottom flask equipped with a magnetic stirrer was dissolved tert-butyl 4-(3-(2-(difluoromethoxy)-6-methoxypyridin-3-yl)-1-(2-isopropylphenyl)ureido)piperidine-1-carboxylate (1 equiv) from the previous step in dichloromethane (0.15 M). To this solution was then added trifluoroacetic acid (150 equiv, Sigma-Aldrich) and the resulting reaction mixture was stirred at RT for 20 min. The volatiles were then removed in vacuo via sequential azeotropic distillation with toluene and heptane. The residue thus obtained was then partitioned between EtOAc and saturated aq. NaHCO₃. The aqueous layer was separated and back extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→EtOAc) afforded 3-(2-(difluoromethoxy)-6-methoxypyridin-3-yl)-1-(2-isopropylphenyl)-1-(piperidin-4-yl)urea as a white solid (77% yield).

Step 3: In a round-bottom flask equipped with a magnetic stirrer was combined 3-(2-(difluoromethoxy)-6-methoxypyridin-3-yl)-1-(2-isopropylphenyl)-1-(piperidin-4-yl)urea (1 equiv) from the previous step and triethylamine (3 equiv, Sigma-Aldrich) in dichloromethane (0.063 M). To this was then added methyl chloroformate (2 equiv, Sigma-Aldrich), neat and dropwise, over a period of 5 min. After 2 h of stirring at RT, the reaction was quenched with water and extracted with DCM. The combined organic extracts were washed further with brine, dried over Na₂SO₄, filtered, and the filtrate concentrated in vacuo. Purification of the crude product thus obtained by way of reverse-phase column chromatography (C₁₈, gradient elution: 9:1 (v/v) H₂O: MeCN→MeCN) afforded the title compound as a white solid (40% yield). LCMS: m/z=493.2 [M+H]⁺; ¹H NMR (methanol-d₄): δ=8.06 (d, J=8.4 Hz, 1H), 7.56˜7.17 (m, 5H), 6.53 (d, J=8.4 Hz, 1H), 4.47˜4.39 (m, 1H), 4.19˜4.11 (m, 2H), 3.82 (s, 3H), 3.63 (s, 3H), 3.24˜3.18 (m, 1H), 2.89 (br s, 2H), 2.08 (br d, J=12.4 Hz, 1H), 1.83 (br d, J=12.4 Hz, 1H), 1.63˜1.55 (m, 1H), 1.29˜1.17 (m, 7H).

The following example was prepared in an analogous fashion to Example 63, but substituting methyl chloroformate with the requisite electrophile.

| | Starting | | LCMS |
|---|---|---|---|
| Example | Electrophile | Structure | (m/z) |
| 64 | 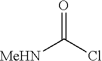 | 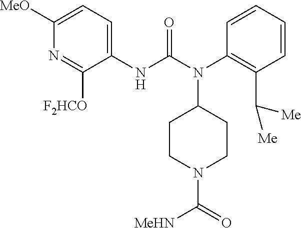 [SMILES: CNC(=O)N1CCC(N(C(=O)Nc2ccc(OC)nc2OC(F)F)c2ccccc2C(C)C)CC1] | 492.3 [M + H]⁺ |
| | Combi-Blocks | 4-(3-(2-(difluoromethoxy)-6-methoxypyridin- | |
| | | 3-yl)-1-(2-isopropylphenyl)ureido)-N- | |
| | | methylpiperidine-1-carboxamide | |

### Example 65: 4-(3-(2-(difluoromethoxy)-6-methoxypyridin-3-yl)-1-(2-isopropylphenyl)ureido)piperidine-1-carboxamide

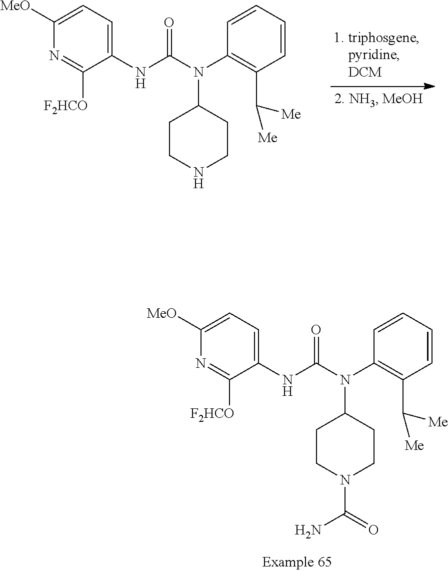 [SMILES: COc1ccc(NC(=O)N(c2ccccc2C(C)C)C2CCNCC2)c(OC(F)F)n1 ; COc1ccc(NC(=O)N(c2ccccc2C(C)C)C2CCN(C(N)=O)CC2)c(OC(F)F)n1]

In a round-bottom flask equipped with a magnetic stirrer was combined 3-(2-(difluoromethoxy)-6-methoxypyridin-3-yl)-1-(2-isopropylphenyl)-1-(piperidin-4-yl)urea (1 equiv, Example 63, step 2) and pyridine (3 equiv, Sigma-Aldrich) in dichloromethane (0.046 M). To this was then added sequentially and portionwise at 0° C., triphosgene (0.35 equiv, Sigma-Aldrich) and ammonia (7 M solution in methanol, 3 equiv, Sigma-Aldrich). The resulting mixture was first warmed to RT over 10 min, and then heated at 50° C. for 1 h. The volatiles were then removed in vacuo and the crude product thus obtained was purified by way of reverse-phase column chromatography (C₁₈, gradient elution: 9:1 (v/v) H₂O: MeCN→MeCN) to afford the title compound as a white solid (54% yield). LCMS: m/z=478.2 [M+H]⁺; ¹H NMR (methanol-d₄): δ=8.05 (d, J=8.7 Hz, 1H), 7.60˜7.12 (m, 5H), 6.54 (d, J=8.7 Hz, 1H), 4.52˜4.43 (m, 1H), 4.11˜4.01 (m, 2H), 3.82 (s, 3H), 3.22 (sept, J=6.9 Hz, 1H), 2.88 (td, J=10.8, 2.7 Hz, 2H), 2.08 (br d, J=12.3 Hz, 1H), 1.82 (br d, J=12.3 Hz, 1H), 1.59 (qd, J=12.0, 3.9 Hz, 1H), 1.30˜1.20 (m, 7H).

### Example 66: Preparation of 1-(1-acetylpiperidin-4-yl)-3-(2-(difluoromethoxy)-6-methylpyridin-3-yl)-1-(2-isopropylphenyl)urea

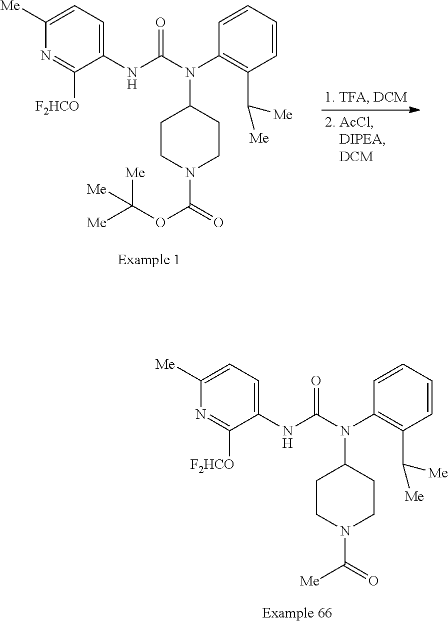 [SMILES: Cc1ccc(NC(=O)N(c2ccccc2C(C)C)C2CCN(C(=O)OC(C)(C)C)CC2)c(OC(F)F)n1 ; CC(=O)N1CCC(N(C(=O)Nc2ccc(C)nc2OC(F)F)c2ccccc2C(C)C)CC1]

In a round-bottom flask equipped with a magnetic stirrer was dissolved Example 1 (1 equiv) in dichloromethane (0.15 M). To this solution was then added trifluoroacetic acid (150 equiv, Sigma-Aldrich) and the resulting reaction mixture was stirred at RT for 20 min. The volatiles were then removed in vacuo via sequential azeotropic distillation with toluene and heptane. The residue thus obtained was then partitioned between EtOAc and saturated aq. NaHCO₃. The aqueous layer was separated and back extracted with EtOAc. The combined organic extracts were washed further with water and brine, dried over MgSO₄, filtered, and the filtrate concentrated in vacuo. The crude 3-(2-(difluoromethoxy)-6-methylpyridin-3-yl)-1-(2-isopropylphenyl)-1-(piperidin-4-yl)urea thus obtained was then re-taken up in dichloromethane (0.04 M), and added sequentially N,N-diisopropylethylamine (3 equiv, Sigma-Aldrich) and acetyl chloride (1.8 equiv, Sigma-Aldrich). After 40 min of stirring at RT, the reaction was quenched with water and extracted with DCM. The combined organic extracts were washed further with brine, dried over MgSO₄, filtered, and the filtrate concentrated in vacuo. Purification of the crude product thus obtained by way of column chromatography (SiO₂, gradient elution: Hex→EtOAc) afforded the title compound as a white solid (83% yield). LCMS: m/z=461.1 [M+H]⁺; ¹H NMR (DMSO-d₆): δ=8.30 (d, J=8.5 Hz, 1H), 7.58 (d, J=9.0 Hz, 1H), 7.50 (t, J=8.0 Hz, 1H), 7.44 (t, J=73.0 Hz, 1H), 7.35 (t, J=7.5 Hz, 1H), 7.25 (d, J=7.5 Hz, 1H), 7.04 (d, J=8.5 Hz, 1H), 6.32 (s, 1H), 4.46˜4.39 (m, 2H), 3.87˜3.81 (m, 1H), 3.12˜3.07 (m, 2H), 2.57˜2.53 (m, 1H), 2.31 (s, 3H), 2.04˜2.00 (m, 1H), 1.94 (s, 1.5H, rotamer A), 1.91 (s, 1.5H, rotamer B), 1.76˜1.74 (m, 1H), 1.56˜1.32 (m, 1H), 1.22 (d, J=7.0 Hz, 1.5H, rotamer A), 1.20 (d, J=7.0 Hz, 1.5H, rotamer B), 1.19˜0.96 (m, 4H).

The following examples were prepared in an analogous fashion to Example 66, but substituting Example 1 with the requisite carbamate. In Example 266, Example 267, and Example 271, only 0.8 equiv of acetyl chloride was used to minimize the formation of bis-acylation products. In Example 268, acetyl chloride (1.8 equiv) was also substituted with 1,3-thiazole-2-carbonyl chloride (1.1 equiv, Combi-Blocks).

| | | | LCMS |
|---|---|---|---|
| Example | Starting Carbamate | Structure | (m/z) |
| 67 | 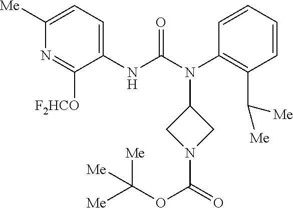 [SMILES: Cc1ccc(NC(=O)N(c2ccccc2C(C)C)C2CN(C(=O)OC(C)(C)C)C2)c(OC(F)F)n1] | 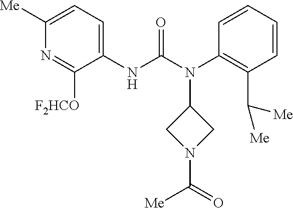 [SMILES: CC(=O)N1CC(N(C(=O)Nc2ccc(C)nc2OC(F)F)c2ccccc2C(C)C)C1] | 432.8 [M + H]⁺ |
| | Example 2 | 1-(1-acetylazetidin-3-yl)-3-(2- | |
| | | (difluoromethoxy)-6-methylpyridin-3-yl)-1- | |
| | | (2-isopropylphenyl)urea | |
| 68 | 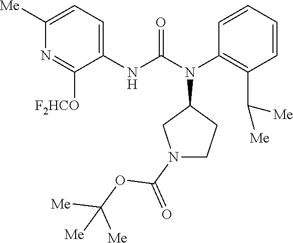 [SMILES: Cc1ccc(NC(=O)N(c2ccccc2C(C)C)[C@H]2CCN(C(=O)OC(C)(C)C)C2)c(OC(F)F)n1] | 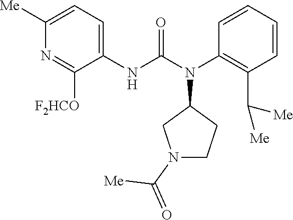 [SMILES: CC(=O)N1CC[C@H](N(C(=O)Nc2ccc(C)nc2OC(F)F)c2ccccc2C(C)C)C1] | 447.1 [M + H]⁺ |
| | Example 8 | (S)-1-(1-acetylpyrrolidin-3-yl)-3-(2- | |
| | | (difluoromethoxy)-6-methylpyridin-3-yl)-1- | |
| | | (2-isopropylphenyl)urea | |
| 69 | 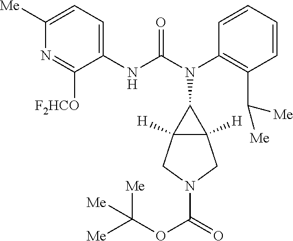 [SMILES: Cc1ccc(NC(=O)N(c2ccccc2C(C)C)[C@H]2[C@@H]3CN(C(=O)OC(C)(C)C)C[C@@H]32)c(OC(F)F)n1] | 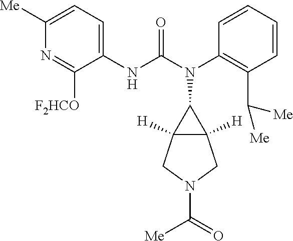 [SMILES: CC(=O)N1C[C@@H]2[C@H](C1)[C@H]2N(C(=O)Nc1ccc(C)nc1OC(F)F)c1ccccc1C(C)C] | 459.1 [M + H]⁺ |
| | Example 9 | 1-((1R,5S,6s)-3-acetyl-3- | |
| | | azabicyclo[3.1.0]hexan-6-yl)-3-(2- | |
| | | (difluoromethoxy)-6-methylpyridin-3-yl)-1- | |
| | | (2-isopropylphenyl)urea | |
| 70 | 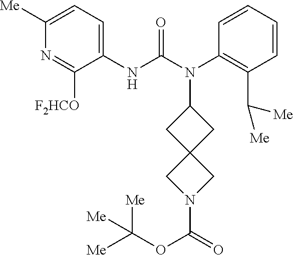 [SMILES: Cc1ccc(NC(=O)N(c2ccccc2C(C)C)C2CC3(C2)CN(C(=O)OC(C)(C)C)C3)c(OC(F)F)n1] | 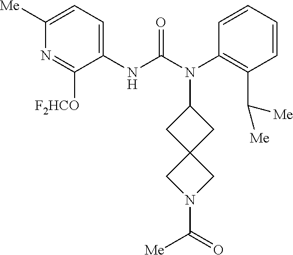 [SMILES: CC(=O)N1CC2(CC(N(C(=O)Nc3ccc(C)nc3OC(F)F)c3ccccc3C(C)C)C2)C1] | 473.1 [M + H]⁺ |
| | Example 10 | 1-(2-acetyl-2-azaspiro[3.3]heptan-6-yl)-3- | |
| | | (2-(difluoromethoxy)-6-methylpyridin-3- | |
| | | yl)-1-(2-isopropylphenyl)urea | |
| 71 | 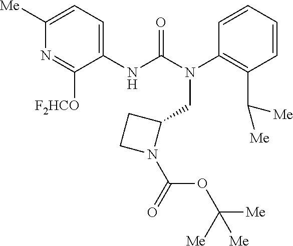 [SMILES: Cc1ccc(NC(=O)N(C[C@H]2CCN2C(=O)OC(C)(C)C)c2ccccc2C(C)C)c(OC(F)F)n1] | 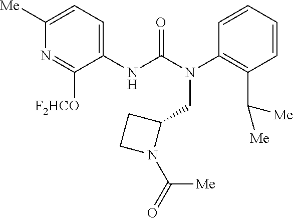 [SMILES: CC(=O)N1CC[C@@H]1CN(C(=O)Nc1ccc(C)nc1OC(F)F)c1ccccc1C(C)C] | 447.1 [M + H]⁺ |
| | Example 11 | (R)-1-((1-acetylazetidin-2-yl)methyl)-3-(2- | |
| | | (difluoromethoxy)-6-methylpyridin-3-yl)-1- | |
| | | (2-isopropylphenyl)urea | |
| 72 | 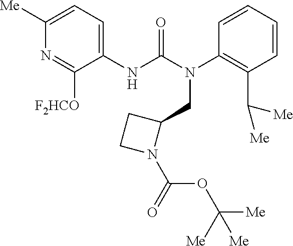 [SMILES: Cc1ccc(NC(=O)N(C[C@@H]2CCN2C(=O)OC(C)(C)C)c2ccccc2C(C)C)c(OC(F)F)n1] | 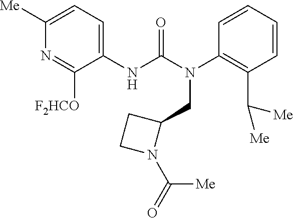 [SMILES: CC(=O)N1CC[C@H]1CN(C(=O)Nc1ccc(C)nc1OC(F)F)c1ccccc1C(C)C] | 447.1 [M + H]⁺ |
| | Example 12 | | |
| | | (S)-1-((1-acetylazetidin-2-yl)methyl)-3-(2- | |
| | | (difluoromethoxy)-6-methylpyridin-3-yl)-1- | |
| | | (2-isopropylphenyl)urea | |
| 264 | 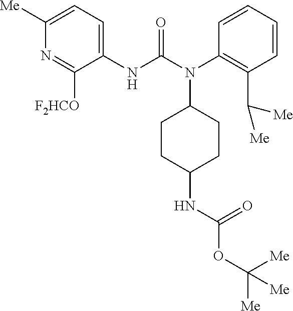 [SMILES: Cc1ccc(NC(=O)N(c2ccccc2C(C)C)C2CCC(NC(=O)OC(C)(C)C)CC2)c(OC(F)F)n1] | 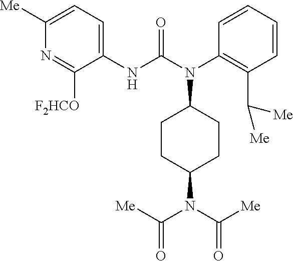 [SMILES: CC(=O)N(C(C)=O)[C@H]1CC[C@@H](N(C(=O)Nc2ccc(C)nc2OC(F)F)c2ccccc2C(C)C)CC1] | 517.2 [M + H]⁺ |
| | Example 263 | N-acetyl-N-((1s,4s)-4-(3-(2- | |
| | | (difluoromethoxy)-6-methylpyridin-3-yl)- | |
| | | 1-(2-isopropylphenyl)ureido)- | |
| | | cyclohexyl)acetamide | |
| 265 | 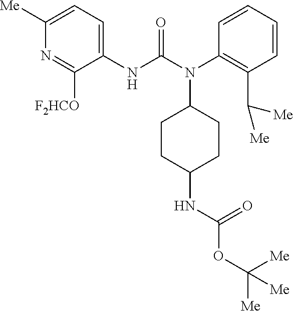 [SMILES: Cc1ccc(NC(=O)N(c2ccccc2C(C)C)C2CCC(NC(=O)OC(C)(C)C)CC2)c(OC(F)F)n1] | 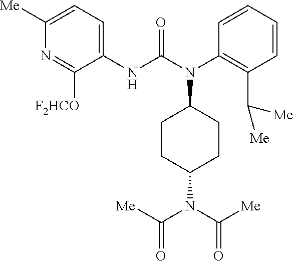 [SMILES: CC(=O)N(C(C)=O)[C@H]1CC[C@H](N(C(=O)Nc2ccc(C)nc2OC(F)F)c2ccccc2C(C)C)CC1] | 517.2 [M + H]⁺ |
| | Example 263 | N-acetyl-N-((1r,4r)-4-(3-(2- | |
| | | (difluoromethoxy)-6-methylpyridin-3-yl)- | |
| | | 1-(2-isopropylphenyl)ureido)- | |
| | | cyclohexyl)acetamide | |
| 266 | 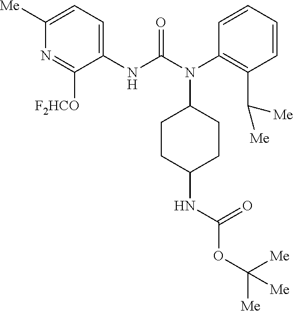 [SMILES: Cc1ccc(NC(=O)N(c2ccccc2C(C)C)C2CCC(NC(=O)OC(C)(C)C)CC2)c(OC(F)F)n1] | 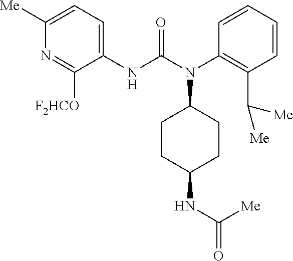 [SMILES: CC(=O)N[C@H]1CC[C@@H](N(C(=O)Nc2ccc(C)nc2OC(F)F)c2ccccc2C(C)C)CC1] | 475.1 [M + H]⁺ |
| | Example 263 | N-((1s,4s)-4-(3-(2-(difluoromethoxy)-6- | |
| | | methylpyridin-3-yl)-1-(2- | |
| | | isopropylphenyl)ureido)cyclohexyl) | |
| | | acetamide | |
| 267 | 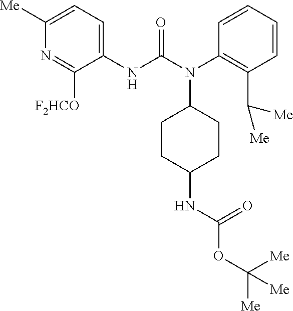 [SMILES: Cc1ccc(NC(=O)N(c2ccccc2C(C)C)C2CCC(NC(=O)OC(C)(C)C)CC2)c(OC(F)F)n1] | 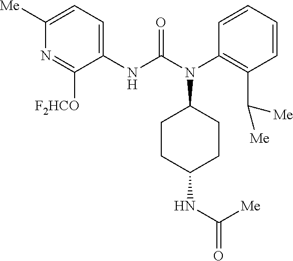 [SMILES: CC(=O)N[C@H]1CC[C@H](N(C(=O)Nc2ccc(C)nc2OC(F)F)c2ccccc2C(C)C)CC1] | 475.1 [M + H]⁺ |
| | Example 263 | N-((1r,4r)-4-(3-(2-(difluoromethoxy)-6- | |
| | | methylpyridin-3-yl)-1-(2- | |
| | | isopropylphenyl)ureido)cyclohexyl) | |
| | | acetamide | |
| 268 | 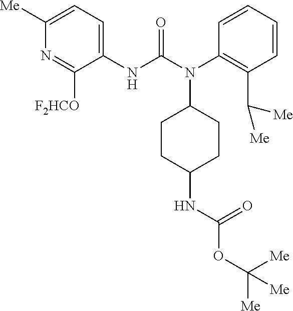 [SMILES: Cc1ccc(NC(=O)N(c2ccccc2C(C)C)C2CCC(NC(=O)OC(C)(C)C)CC2)c(OC(F)F)n1] | 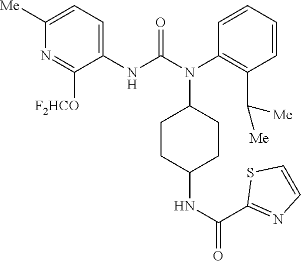 [SMILES: Cc1ccc(NC(=O)N(c2ccccc2C(C)C)C2CCC(NC(=O)c3nccs3)CC2)c(OC(F)F)n1] | 544.1 [M + H]⁺ |
| | Example 263 | N-(4-(3-(2-(difluoromethoxy)-6- | |
| | | methylpyridin-3-yl)-1-(2- | |
| | | isopropylphenyl)ureido)cyclohexyl)thiazole- | |
| | | 2-carboxamide | |
| 271 | 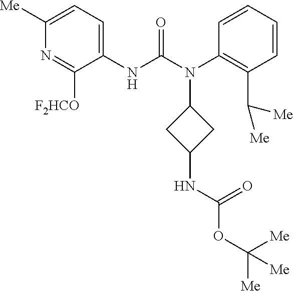 [SMILES: Cc1ccc(NC(=O)N(c2ccccc2C(C)C)C2CC(NC(=O)OC(C)(C)C)C2)c(OC(F)F)n1] | 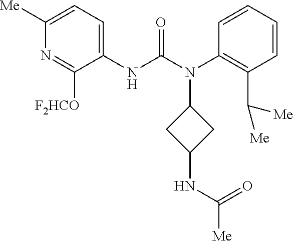 [SMILES: CC(=O)NC1CC(N(C(=O)Nc2ccc(C)nc2OC(F)F)c2ccccc2C(C)C)C1] | 446.8 [M + H]⁺ |
| | Example 270 | N-(3-(3-(2-(difluoromethoxy)-6- | |
| | | methylpyridin-3-yl)-1-(2- | |
| | | isopropylphenyl)ureido)cyclobutyl) | |
| | | acetamide | |

### Example 73: Preparation of 3-(2-(difluoromethoxy)-6-methylpyridin-3-yl)-1-(2-isopropylphenyl)-1-(1-(methylsulfonyl)piperidin-4-yl)urea

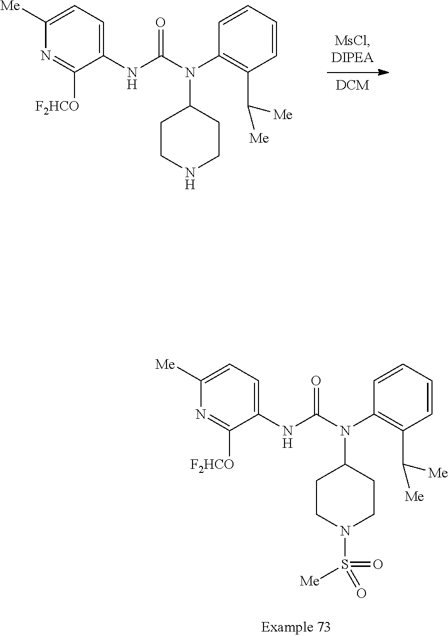 [SMILES: Cc1ccc(NC(=O)N(c2ccccc2C(C)C)C2CCNCC2)c(OC(F)F)n1 ; Cc1ccc(NC(=O)N(c2ccccc2C(C)C)C2CCN(S(C)(=O)=O)CC2)c(OC(F)F)n1]

In a round-bottom flask equipped with a magnetic stirrer was combined 3-(2-(difluoromethoxy)-6-methylpyridin-3-yl)-1-(2-isopropylphenyl)-1-(piperidin-4-yl)urea (1 equiv, Example 66, step 1) and N,N-diisopropylethylamine (3 equiv, Sigma-Aldrich) in dichloromethane (0.04 M). To this was then added methanesulfonyl chloride (1.2 equiv, Sigma-Aldrich). After 5 min of stirring at RT, the reaction was quenched with water and extracted with DCM. The combined organic extracts were washed further with brine, dried over MgSO₄, filtered, and the filtrate concentrated in vacuo. Purification of the crude product thus obtained by way of column chromatography (SiO₂, gradient elution: Hex→EtOAc) afforded the title compound as a white solid (84% yield). LCMS: m/z=497.0 [M+H]⁺; ¹H NMR (DMSO-d₆): δ=8.29 (d, J=8.5 Hz, 1H), 7.58 (dd, J=8.0, 1.5 Hz, 1H), 7.52 (t, J=8.0 Hz, 1H), 7.44 (t, J=73.0 Hz, 1H), 7.37 (td, J=7.5, 1.5 Hz, 1H), 7.28 (dd, J=8.0, 1.5 Hz, 1H), 7.05 (d, J=8.0 Hz, 1H), 6.35 (s, 1H), 4.32 (tt, J=12.0, 3.5 Hz, 1H), 3.62˜3.55 (m, 2H), 3.09 (sept, J=7.0 Hz, 1H), 2.84˜2.80 (m, 5H), 2.31 (s, 3H), 2.14˜2.09 (m, 1H), 1.87˜1.82 (m, 1H), 1.63 (qd, J=12.0, 4.0 Hz, 1H), 1.25 (qd, J=12.5, 4.5 Hz, 1H), 1.22 (d, J=7.0 Hz, 3H), 1.09 (d, J=7.0 Hz, 3H).

The following examples were prepared in an analogous fashion to Example 73, but substituting 3-(2-(difluoromethoxy)-6-methylpyridin-3-yl)-l-(2-isopropylphenyl)-1-(piperidin-4-yl)urea with the requisite amine.

| | | | LCMS |
|---|---|---|---|
| Example | Starting Amine | Structure | (m/z) |
| 74 | 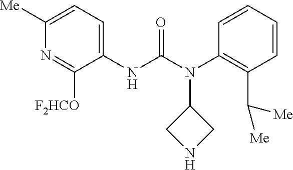 [SMILES: Cc1ccc(NC(=O)N(c2ccccc2C(C)C)C2CNC2)c(OC(F)F)n1] | 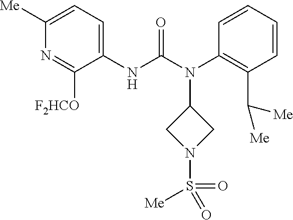 [SMILES: Cc1ccc(NC(=O)N(c2ccccc2C(C)C)C2CN(S(C)(=O)=O)C2)c(OC(F)F)n1] | 469.1 [M + H]⁺ |
| | Example 67, step 1 | 3-(2-(difluoromethoxy)-6- | |
| | | methylpyridin-3-yl)-1-(2- | |
| | | isopropylphenyl)-1-(1- | |
| | | (methylsulfonyl)azetidin-3-yl)urea | |
| 75 | 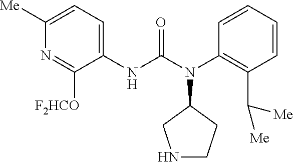 [SMILES: Cc1ccc(NC(=O)N(c2ccccc2C(C)C)[C@H]2CCNC2)c(OC(F)F)n1] | 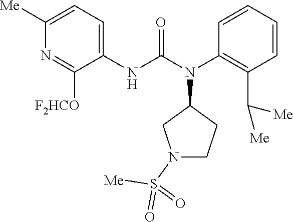 [SMILES: Cc1ccc(NC(=O)N(c2ccccc2C(C)C)[C@H]2CCN(S(C)(=O)=O)C2)c(OC(F)F)n1] | 483.0 [M + H]⁺ |
| | Example 68, step 1 | (S)-3-(2-(difluoromethoxy)-6- | |
| | | methylpyridin-3-yl)-1-(2- | |
| | | isopropylphenyl)-1-(1- | |
| | | (methylsulfonyl)pyrrolidin-3-yl)urea | |
| 76 | 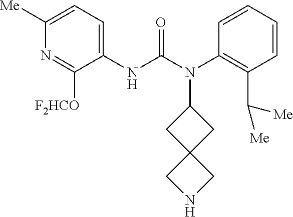 [SMILES: Cc1ccc(NC(=O)N(c2ccccc2C(C)C)C2CC3(CNC3)C2)c(OC(F)F)n1] | 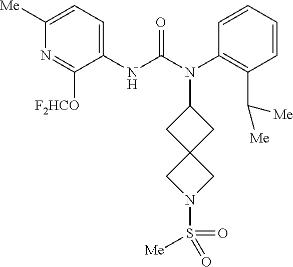 [SMILES: Cc1ccc(NC(=O)N(c2ccccc2C(C)C)C2CC3(C2)CN(S(C)(=O)=O)C3)c(OC(F)F)n1] | 509.1 [M + H]⁺ |
| | Example 70, step 1 | 3-(2-(difluoromethoxy)-6- | |
| | | methylpyridin-3-yl)-1-(2- | |
| | | isopropylphenyl)-1-(2- | |
| | | (methylsulfonyl)-2- | |
| | | azaspiro[3.3]heptan-6-yl)urea | |
| 269 | 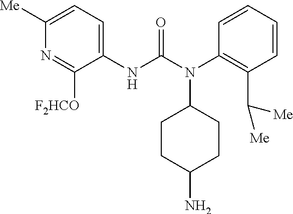 [SMILES: Cc1ccc(NC(=O)N(c2ccccc2C(C)C)C2CCC(N)CC2)c(OC(F)F)n1] | 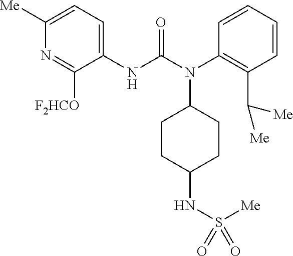 [SMILES: Cc1ccc(NC(=O)N(c2ccccc2C(C)C)C2CCC(NS(C)(=O)=O)CC2)c(OC(F)F)n1] | 510.8 [M + H]⁺ |
| | Example 268, step 1 | N-(4-(3-(2-(difluoromethoxy)-6- | |
| | | methylpyridin-3-yl)-1-(2- | |
| | | isopropylphenyl)ureido)cyclohexyl) | |
| | | methanesulfonamide | |
| 272 | 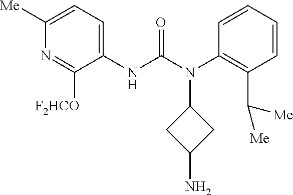 [SMILES: Cc1ccc(NC(=O)N(c2ccccc2C(C)C)C2CC(N)C2)c(OC(F)F)n1] | 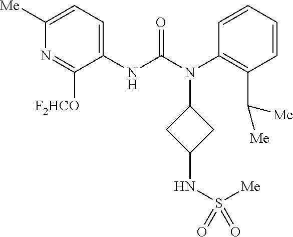 [SMILES: Cc1ccc(NC(=O)N(c2ccccc2C(C)C)C2CC(NS(C)(=O)=O)C2)c(OC(F)F)n1] | 482.8 [M + H]⁺ |
| | Example 271, step 1 | N-(3-(3-(2-(difluoromethoxy)-6- | |
| | | methylpyridin-3-yl)-1-(2- | |
| | | isopropylphenyl)ureido)cyclobutyl) | |
| | | methanesulfonamide | |

### Example 77: methyl 3-(3-(2-(difluoromethoxy)-6-methylpyridin-3-yl)-l-(2-isopropylphenyl)ureido)azetidine-1-carboxylate

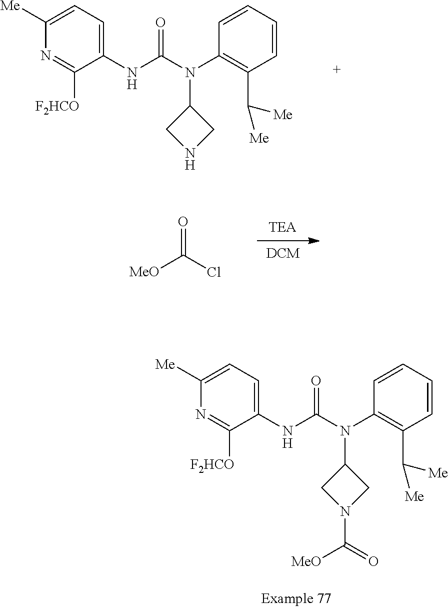 [SMILES: Cc1ccc(NC(=O)N(c2ccccc2C(C)C)C2CNC2)c(OC(F)F)n1 ; COC(=O)Cl ; COC(=O)N1CC(N(C(=O)Nc2ccc(C)nc2OC(F)F)c2ccccc2C(C)C)C1]

In a round-bottom flask equipped with a magnetic stirrer was combined 1-(azetidin-3-yl)-3-(2-(difluoromethoxy)-6-methylpyridin-3-yl)-l-(2-isopropylphenyl)urea (1 equiv, Example 67, step 1) and triethylamine (3 equiv, Sigma-Aldrich) in dichloromethane (0.03 M). To this was then added at 0° C. methyl chloroformate (1.1 equiv, Sigma-Aldrich), neat and dropwise, over a period of 5 min. After 2 h of stirring at 0° C., the reaction was quenched with water and extracted with DCM. The combined organic extracts were washed further with brine, dried over MgSO₄, filtered, and the filtrate concentrated in vacuo. Purification of the crude product thus obtained by way of reverse-phase column chromatography (C₁₈, gradient elution: 9:1 (v/v) H₂O: MeCN→MeCN) afforded the title compound as a white solid (34% yield). LCMS: m/z=449.2 [M+H]⁺; ¹H NMR (DMSO-d₆): δ=8.17 (d, J=7.8 Hz, 1H), 7.61˜7.51 (m, 2H), 7.48 (t, J=72.0 Hz, 1H), 7.41˜7.37 (m, 2H), 7.06 (d, J=8.1 Hz, 1H), 6.59 (s, 1H), 4.92˜4.86 (m, 1H), 4.13˜4.08 (m, 2H), 3.91˜3.86 (m, 1H), 3.73˜3.68 (m, 1H), 3.51 (s, 3H), 2.98 (sept, J=6.9 Hz, 1H), 2.33 (s, 3H), 1.12 (d, J=6.9 Hz, 6H).

The following example was prepared in an analogous fashion to Example 77, but substituting methyl chloroformate with the requisite electrophile.

| | Starting | | LCMS |
|---|---|---|---|
| Example | Electrophile | Structure | (m/z) |
| 78 | 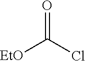 | 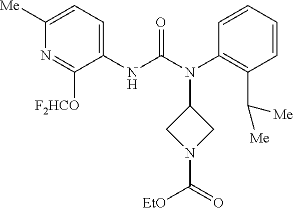 [SMILES: CCOC(=O)N1CC(N(C(=O)Nc2ccc(C)nc2OC(F)F)c2ccccc2C(C)C)C1] | 463.2 [M + H]⁺ |
| | Sigma-Aldrich | ethyl 3-(3-(2-(difluoromethoxy)-6- | |
| | | methylpyridin-3-yl)-1-(2- | |
| | | isopropylphenyl)ureido)azetidine-1-carboxylate | |

### Example 79: Preparation of 1-(1-(2-(2H-1,2,3-triazol-2-yl)acetyl)piperidin-4-yl)-3-(2-(difluoromethoxy)-6-methylpyridin-3-yl)-1-(2-isopropylphenyl)urea

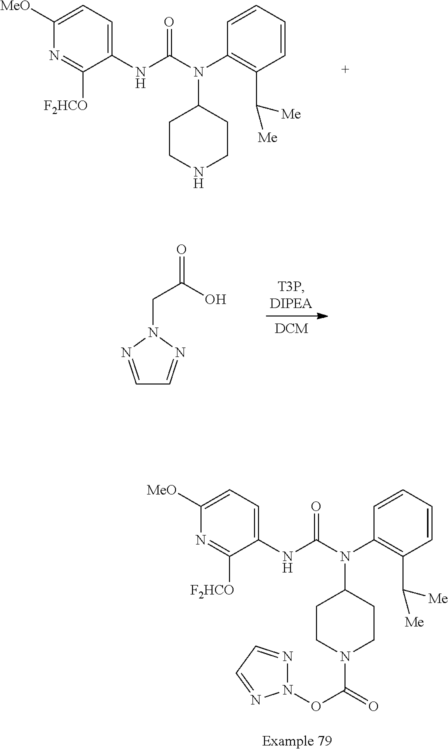 [SMILES: COc1ccc(NC(=O)N(c2ccccc2C(C)C)C2CCNCC2)c(OC(F)F)n1 ; O=C(O)Cn1nccn1 ; COc1ccc(NC(=O)N(c2ccccc2C(C)C)C2CCN(C(=O)On3nccn3)CC2)c(OC(F)F)n1]

In a dried, round-bottom flask equipped with a magnetic stirrer was combined 3-(2-(difluoromethoxy)-6-methylpyridin-3-yl)-1-(2-isopropylphenyl)-1-(piperidin-4-yl)urea (1 equiv, Example 66, step 1), 2-(triazol-2-yl)acetic acid (2 equiv, Combi-Blocks), and N,N-diisopropylethylamine (3 equiv, Sigma-Aldrich) in dichloromethane (0.04 M). To this reaction mixture was then added propylphosphonic anhydride (50% w/w solution in EtOAc, 4 equiv, Sigma-Aldrich) and the resulting solution was stirred at RT for 2 h. The reaction was then quenched with water and extracted with DCM. The combined organic extracts were washed further with 1 M aq. NaOH, water and brine, dried over MgSO₄, filtered, and the filtrate concentrated in vacuo. Purification of the crude product thus obtained by way of column chromatography (SiO₂, gradient elution: Hex→EtOAc) afforded the title compound as a white solid (71% yield). LCMS: m/z=528.1 [M+H]⁺; ¹H NMR (DMSO-d₆): δ=8.31 (dd, J=8.0, 2.0 Hz, 1H), 7.76 (s, 1H), 7.73 (s, 1H), 7.59 (d, J=8.0 Hz, 1H), 7.52 (t, J=8.0 Hz, 1H), 7.44 (t, J=73.0 Hz, 1H), 7.39˜7.35 (m, 1H), 7.26 (t, J=6.5 Hz, 1H), 7.05 (d, J=8.0 Hz, 1H), 6.33 (br s, 1H), 5.57˜5.37 (m, 2H), 4.96˜4.32 (m, 2H), 3.94˜3.87 (m, 1H), 3.16˜3.08 (m, 2H), 2.70˜2.65 (m, 2H), 2.31 (s, 3H), 2.07˜2.01 (m, 1H), 1.82˜1.73 (m, 1H), 1.61˜1.43 (m, 1H), 1.24 (d, J=7.0 Hz, 1.5H, rotamer A), 1.21 (d, J=7.0 Hz, 1.5H, rotamer B), 1.09 (d, J=7.0 Hz, 3H).

The following examples were prepared in an analogous fashion to Example 79, but substituting 3-(2-(difluoromethoxy)-6-methylpyridin-3-yl)-1-(2-isopropylphenyl)-1-(piperidin-4-yl)urea with the requisite amine. In Example 81 and Example 85, 2-(triazol-2-yl)acetic acid and propylphosphonic anhydride were also substituted with cyanoacetic acid (Sigma-Aldrich) and HATU (Chem-Impex), respectively. In Example 82, 2-(triazol-2-yl)acetic acid was also substituted with methoxyacetic acid (Sigma-Aldrich).

| | | | LCMS |
|---|---|---|---|
| Example | Starting Amine | Structure | (m/z) |
| 80 | 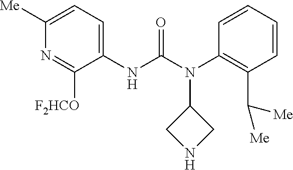 [SMILES: Cc1ccc(NC(=O)N(c2ccccc2C(C)C)C2CNC2)c(OC(F)F)n1] | 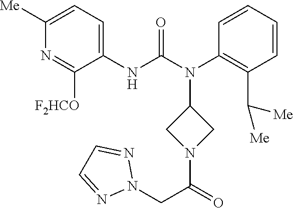 [SMILES: Cc1ccc(NC(=O)N(c2ccccc2C(C)C)C2CN(C(=O)Cn3nccn3)C2)c(OC(F)F)n1] | 500.2 [M + H]⁺ |
| | Example 67, step 1 | 1-(1-(2-(2H-1,2,3-triazol-2- | |
| | | yl)acetyl)azetidin-3-yl)-3-(2- | |
| | | (difluoromethoxy)-6-methylpyridin-3- | |
| | | yl)-1-(2-isopropylphenyl)urea | |
| 81 | 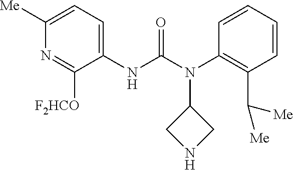 [SMILES: Cc1ccc(NC(=O)N(c2ccccc2C(C)C)C2CNC2)c(OC(F)F)n1] | 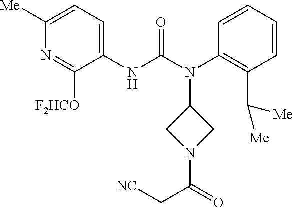 [SMILES: Cc1ccc(NC(=O)N(c2ccccc2C(C)C)C2CN(C(=O)CC#N)C2)c(OC(F)F)n1] | 458.2 [M + H]⁺ |
| | Example 67, step 1 | 1-(1-(2-cyanoacetyl)azetidin-3-yl)-3- | |
| | | (2-(difluoromethoxy)-6-methylpyridin- | |
| | | 3-yl)-1-(2-isopropylphenyl)urea | |
| 82 | 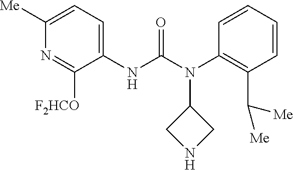 [SMILES: Cc1ccc(NC(=O)N(c2ccccc2C(C)C)C2CNC2)c(OC(F)F)n1] | 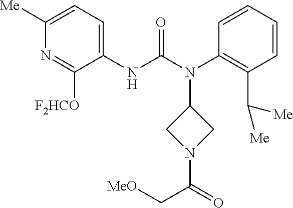 [SMILES: COCC(=O)N1CC(N(C(=O)Nc2ccc(C)nc2OC(F)F)c2ccccc2C(C)C)C1] | 463.2 [M + H]⁺ |
| | Example 67, step 1 | | |
| | | 3-(2-(difluoromethoxy)-6- | |
| | | methylpyridin-3-yl)-1-(2- | |
| | | isopropylphenyl)-1-(1-(2- | |
| | | methoxyacetyl)azetidin-3-yl)urea | |
| 83 | 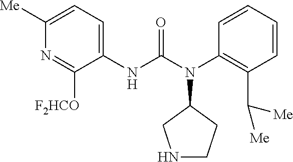 [SMILES: Cc1ccc(NC(=O)N(c2ccccc2C(C)C)[C@H]2CCNC2)c(OC(F)F)n1] | 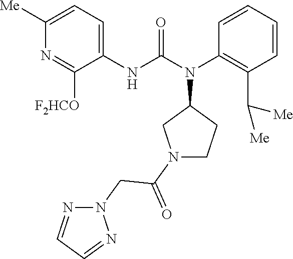 [SMILES: Cc1ccc(NC(=O)N(c2ccccc2C(C)C)[C@H]2CCN(C(=O)Cn3nccn3)C2)c(OC(F)F)n1] | 514.1 [M + H]⁺ |
| | Example 68, step 1 | (S)-1-(1-(2-(2H-1,2,3-triazol-2- | |
| | | yl)acetyl)pyrrolidin-3-yl)-3-(2- | |
| | | (difluoromethoxy)-6-methylpyridin-3- | |
| | | yl)-1-(2-isopropylphenyl)urea | |
| 84 | 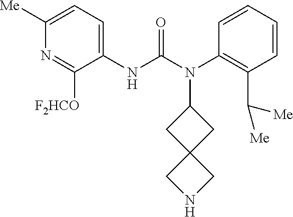 [SMILES: Cc1ccc(NC(=O)N(c2ccccc2C(C)C)C2CC3(CNC3)C2)c(OC(F)F)n1] | 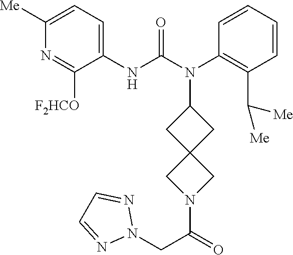 [SMILES: Cc1ccc(NC(=O)N(c2ccccc2C(C)C)C2CC3(C2)CN(C(=O)Cn2nccn2)C3)c(OC(F)F)n1] | 540.2 [M + H]⁺ |
| | Example 70, step 1 | 1-(2-(2-(2H-1,2,3-triazol-2-yl)acetyl)- | |
| | | 2-azaspiro[3.3]heptan-6-yl)-3-(2- | |
| | | (difluoromethoxy)-6-methylpyridin-3- | |
| | | yl)-1-(2-isopropylphenyl)urea | |
| 85 | 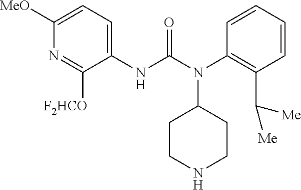 [SMILES: COc1ccc(NC(=O)N(c2ccccc2C(C)C)C2CCNCC2)c(OC(F)F)n1] | 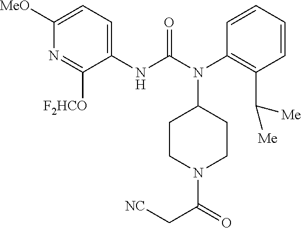 [SMILES: COc1ccc(NC(=O)N(c2ccccc2C(C)C)C2CCN(C(=O)CC#N)CC2)c(OC(F)F)n1] | 502.2 [M + H]⁺ |
| | Example 63, step 2 | 1-(1-(2-cyanoacetyl)piperidin-4-yl)-3- | |
| | | (2-(difluoromethoxy)-6- | |
| | | methoxypyridin-3-yl)-1-(2- | |
| | | isopropylphenyl)urea | |

### Example 86: Preparation of 1-(1-acetylpiperidin-4-yl)-3-(2-(difluoromethoxy)-6-methoxypyridin-3-yl)-1-(2-isopropylphenyl)urea

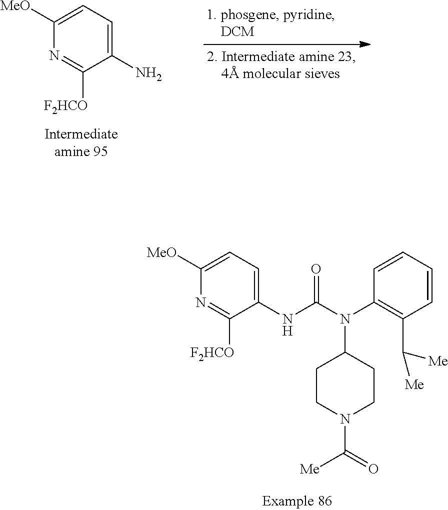 [SMILES: COc1ccc(N)c(OC(F)F)n1 ; COc1ccc(NC(=O)N(c2ccccc2C(C)C)C2CCN(C(C)=O)CC2)c(OC(F)F)n1]

In a dried, round-bottom flask equipped with a magnetic stirrer was combined Intermediate amine 95 (1 equiv) and pyridine (3 equiv, Sigma-Aldrich) in anhydrous dichloromethane (0.1 M). To this was then added phosgene (15% w/w solution in toluene, 1.5 equiv, Sigma-Aldrich) dropwise at RT and the resulting solution was stirred at RT for 30 min. The volatiles were then removed in vacuo and the crude (2-(difluoromethoxy)-6-methoxypyridin-3-yl)carbamic chloride thus obtained was re-taken up in anhydrous dichloromethane (0.1 M). This solution was then added dropwise at RT to another dichloromethane suspension (0.1 M) of Intermediate amine 23 (1 equiv), pyridine (3 equiv, Sigma-Aldrich), and freshly activated 4 Å molecular sieves. The resulting mixture was stirred at RT for 16 h before the reaction was quenched with water. The aqueous layer was separated and back extracted with DCM. The combined organic extracts were dried over MgSO₄, filtered, and the filtrate concentrated in vacuo. Purification of the crude product thus obtained by way of reverse-phase column chromatography (C₁s, gradient elution: 9:1 (v/v) H₂O: MeCN+0.1% formic acid→MeCN+0.1% formic acid) afforded the title compound as a white solid (38% yield). LCMS: m/z=477.1 [M+H]⁺; ¹H NMR (CDCl₃): δ=8.40 (dd, J=9.0, 3.0 Hz, 1H), 7.49˜7.47 (m, 2H), 7.33˜7.28 (m, 1H), 7.17 (t, J=73.0 Hz, 1H), 7.12 (t, J=7.8 Hz, 1H), 6.51 (d, J=8.7 Hz, 1H), 5.96 (s, 1H), 4.71˜4.61 (m, 2H), 3.86˜3.80 (m, 1H), 3.79 (s, 3H), 3.22˜3.10 (m, 2H), 2.61 (q, J=12.6 Hz, 1H), 2.19˜2.02 (m, 4H), 1.92˜1.75 (m, 1H), 1.66˜1.50 (m, 1H), 1.31˜1.14 (in, 7H).

The following examples were prepared in an analogous fashion to Example 86, but substituting Intermediate amine 95 with the requisite amine. In Example 93, Example 99, Example 100, Example 101, Example 104, Example 105, Example 106, Example 110, Example 111, Example 112, Example 113, Example 114, Example 115, and Example 116, phosgene (1.5 equiv) was also substituted with triphosgene (0.5 equiv, Sigma-Aldrich). In Example 106 and Example 110, 4 Å molecular sieves was also substituted with 4-(dimethylamino)pyridine (0.5 equiv, Sigma-Aldrich) and the reaction was heated at 40° C. for an additional 24 h.

| | | | LCMS |
|---|---|---|---|
| Example | Starting Amine | Structure | (m/z) |
| 87 | 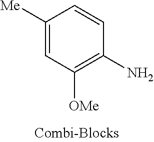 | 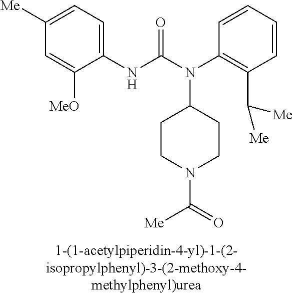 [SMILES: COc1cc(C)ccc1NC(=O)N(c1ccccc1C(C)C)C1CCN(C(C)=O)CC1] | 424.2 [M + H]⁺ |
| 88 | 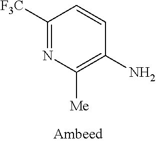 [SMILES: Cc1nc(C(F)(F)F)ccc1N] | 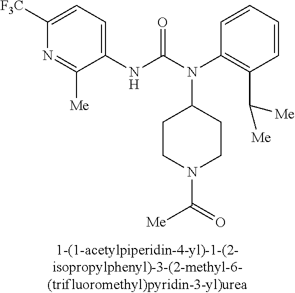 [SMILES: CC(=O)N1CCC(N(C(=O)Nc2ccc(C(F)(F)F)nc2C)c2ccccc2C(C)C)CC1] | 463.1 [M + H]⁺ |
| 89 | 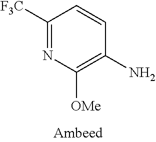 [SMILES: COc1nc(C(F)(F)F)ccc1N] | 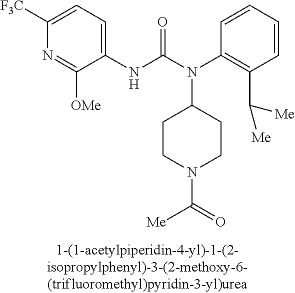 [SMILES: COc1nc(C(F)(F)F)ccc1NC(=O)N(c1ccccc1C(C)C)C1CCN(C(C)=O)CC1] | 479.1 [M + H]⁺ |
| 90 | 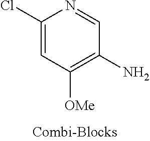 | 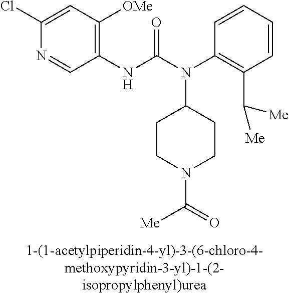 [SMILES: COc1cc(Cl)ncc1NC(=O)N(c1ccccc1C(C)C)C1CCN(C(C)=O)CC1] | 444.8, 446.8 [M + H]⁺ |
| 91 | 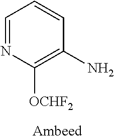 [SMILES: Nc1cccnc1OC(F)F] | 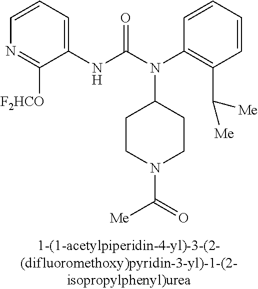 [SMILES: CC(=O)N1CCC(N(C(=O)Nc2cccnc2OC(F)F)c2ccccc2C(C)C)CC1] | 446.8 [M + H]⁺ |
| 92 | 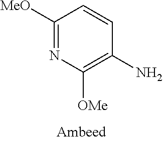 [SMILES: COc1ccc(N)c(OC)n1] | 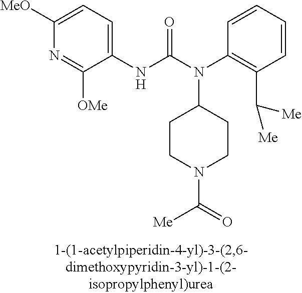 [SMILES: COc1ccc(NC(=O)N(c2ccccc2C(C)C)C2CCN(C(C)=O)CC2)c(OC)n1] | 440.9 [M + H]⁺ |
| 93 | 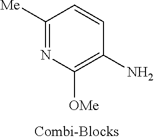 | 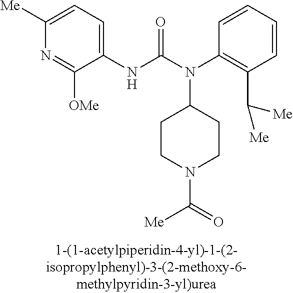 [SMILES: COc1nc(C)ccc1NC(=O)N(c1ccccc1C(C)C)C1CCN(C(C)=O)CC1] | 425.3 [M + H]⁺ |
| 94 | 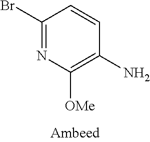 | 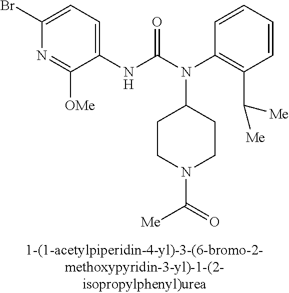 [SMILES: COc1nc(Br)ccc1NC(=O)N(c1ccccc1C(C)C)C1CCN(C(C)=O)CC1] | 488.6, 490.6 [M + H]⁺ |
| 95 | 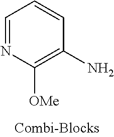 | 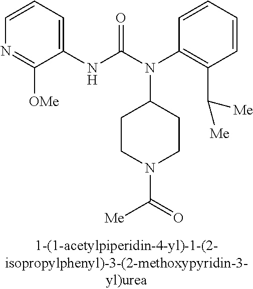 [SMILES: COc1ncccc1NC(=O)N(c1ccccc1C(C)C)C1CCN(C(C)=O)CC1] | 410.9 [M + H]⁺ |
| 96 | 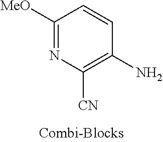 [SMILES: COc1ccc(N)c(C#N)n1] | 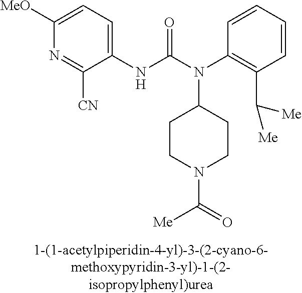 [SMILES: COc1ccc(NC(=O)N(c2ccccc2C(C)C)C2CCN(C(C)=O)CC2)c(C#N)n1] | 436.1 [M + H]⁺ |
| 97 | 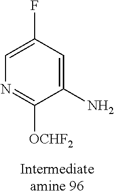 [SMILES: Nc1cc(F)cnc1OC(F)F] | 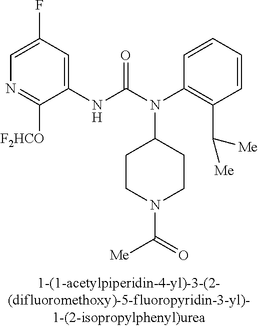 [SMILES: CC(=O)N1CCC(N(C(=O)Nc2cc(F)cnc2OC(F)F)c2ccccc2C(C)C)CC1] | 465.1 [M + H]⁺ |
| 98 | 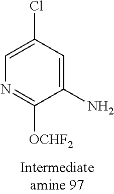 [SMILES: Nc1cc(Cl)cnc1OC(F)F] | 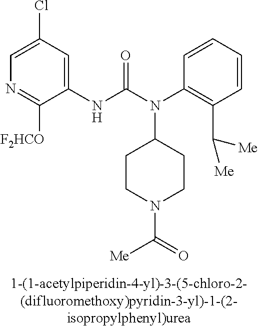 [SMILES: CC(=O)N1CCC(N(C(=O)Nc2cc(Cl)cnc2OC(F)F)c2ccccc2C(C)C)CC1] | 481.0, 483.0 [M + H]⁺ |
| 99 | 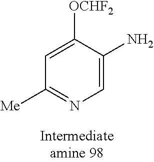 [SMILES: Cc1cc(OC(F)F)c(N)cn1] | 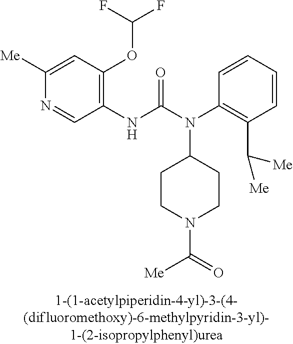 [SMILES: CC(=O)N1CCC(N(C(=O)Nc2cnc(C)cc2OC(F)F)c2ccccc2C(C)C)CC1] | 461.3 [M + H]⁺ |
| 100 | 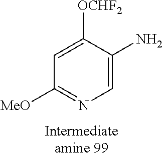 [SMILES: COc1cc(OC(F)F)c(N)cn1] | 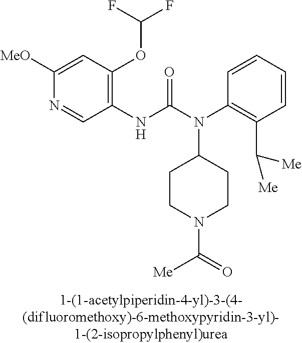 [SMILES: COc1cc(OC(F)F)c(NC(=O)N(c2ccccc2C(C)C)C2CCN(C(C)=O)CC2)cn1] | 477.2 [M + H]⁺ |
| 101 | 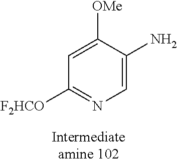 [SMILES: COc1cc(OC(F)F)ncc1N] | 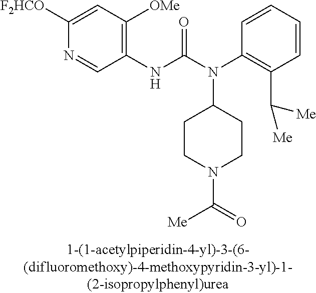 [SMILES: COc1cc(OC(F)F)ncc1NC(=O)N(c1ccccc1C(C)C)C1CCN(C(C)=O)CC1] | 477.3 [M + H]⁺ |
| 102 | 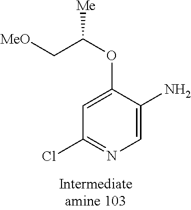 [SMILES: COC[C@H](C)Oc1cc(Cl)ncc1N] | 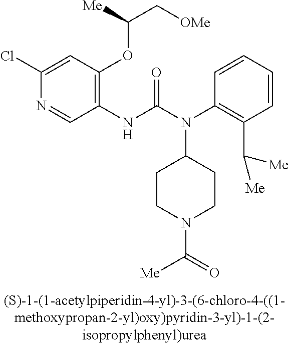 [SMILES: COC[C@H](C)Oc1cc(Cl)ncc1NC(=O)N(c1ccccc1C(C)C)C1CCN(C(C)=O)CC1] | 502.9, 504.8 [M + H]⁺ |
| 103 | 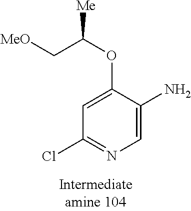 [SMILES: COC[C@@H](C)Oc1cc(Cl)ncc1N] | 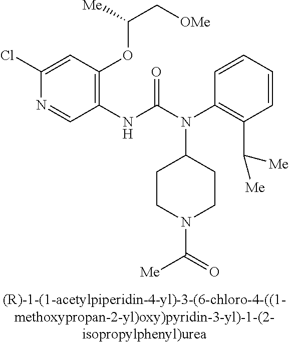 [SMILES: COC[C@@H](C)Oc1cc(Cl)ncc1NC(=O)N(c1ccccc1C(C)C)C1CCN(C(C)=O)CC1] | 502.9, 504.8 [M + H]⁺ |
| 104 | 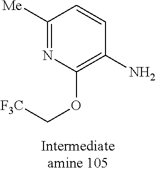 [SMILES: Cc1ccc(N)c(OCC(F)(F)F)n1] | 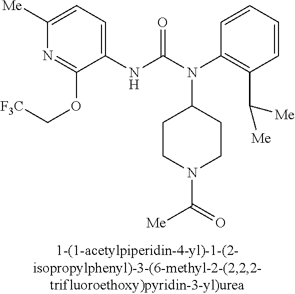 [SMILES: CC(=O)N1CCC(N(C(=O)Nc2ccc(C)nc2OCC(F)(F)F)c2ccccc2C(C)C)CC1] | 493.3 [M + H]⁺ |
| 105 | 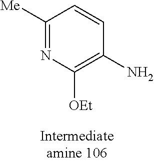 [SMILES: CCOc1nc(C)ccc1N] | 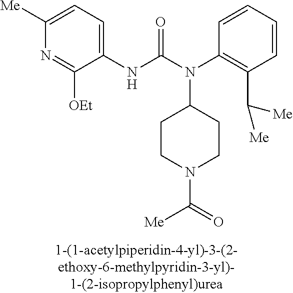 [SMILES: CCOc1nc(C)ccc1NC(=O)N(c1ccccc1C(C)C)C1CCN(C(C)=O)CC1] | 439.3 [M + H]⁺ |
| 106 | 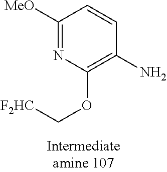 [SMILES: COc1ccc(N)c(OCC(F)F)n1] | 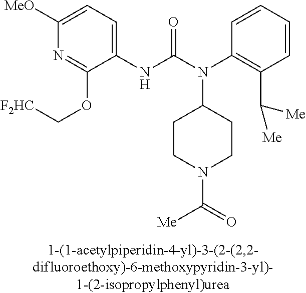 [SMILES: COc1ccc(NC(=O)N(c2ccccc2C(C)C)C2CCN(C(C)=O)CC2)c(OCC(F)F)n1] | 491.3 [M + H]⁺ |
| 107 | 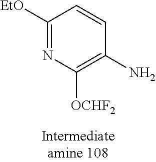 [SMILES: CCOc1ccc(N)c(OC(F)F)n1] | 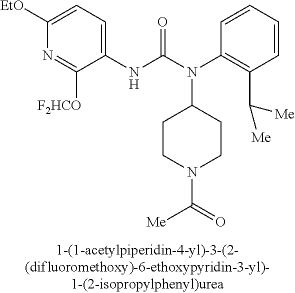 [SMILES: CCOc1ccc(NC(=O)N(c2ccccc2C(C)C)C2CCN(C(C)=O)CC2)c(OC(F)F)n1] | 491.1 [M + H]⁺ |
| 108 | 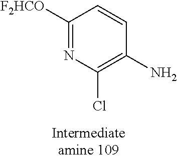 [SMILES: Nc1ccc(OC(F)F)nc1Cl] | 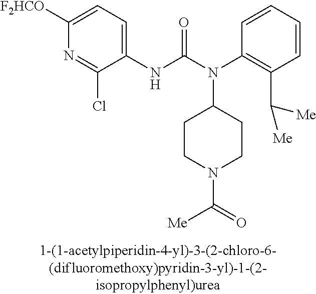 [SMILES: CC(=O)N1CCC(N(C(=O)Nc2ccc(OC(F)F)nc2Cl)c2ccccc2C(C)C)CC1] | 480.8, 482.6 [M + H]⁺ |
| 109 | 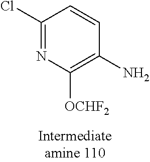 [SMILES: Nc1ccc(Cl)nc1OC(F)F] | 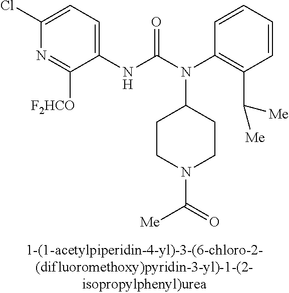 [SMILES: CC(=O)N1CCC(N(C(=O)Nc2ccc(Cl)nc2OC(F)F)c2ccccc2C(C)C)CC1] | 480.8, 482.6 [M + H]⁺ |
| 110 | 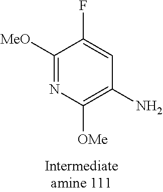 [SMILES: COc1nc(OC)c(F)cc1N] | 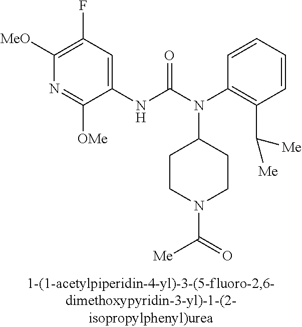 [SMILES: COc1nc(OC)c(NC(=O)N(c2ccccc2C(C)C)C2CCN(C(C)=O)CC2)cc1F] | 459.3 [M + H]⁺ |
| 111 | 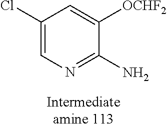 [SMILES: Nc1ncc(Cl)cc1OC(F)F] | 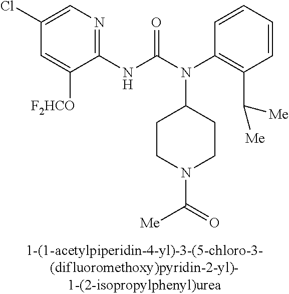 [SMILES: CC(=O)N1CCC(N(C(=O)Nc2ncc(Cl)cc2OC(F)F)c2ccccc2C(C)C)CC1] | 481.2, 483.2 [M + H]⁺ |
| 112 | 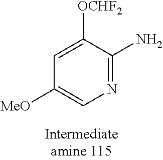 [SMILES: COc1cnc(N)c(OC(F)F)c1] | 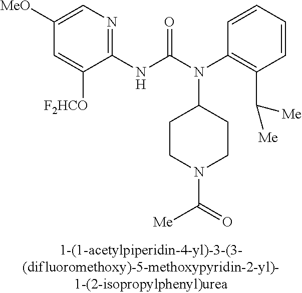 [SMILES: COc1cnc(NC(=O)N(c2ccccc2C(C)C)C2CCN(C(C)=O)CC2)c(OC(F)F)c1] | 477.3 [M + H]⁺ |
| 113 | 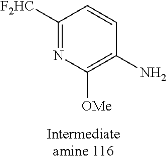 [SMILES: COc1nc(C(F)F)ccc1N] | 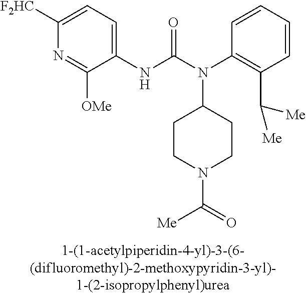 [SMILES: COc1nc(C(F)F)ccc1NC(=O)N(c1ccccc1C(C)C)C1CCN(C(C)=O)CC1] | 461.3 [M + H]⁺ |
| 114 | 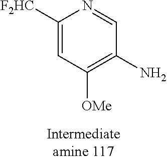 [SMILES: COc1cc(C(F)F)ncc1N] | 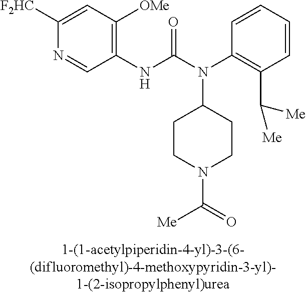 [SMILES: COc1cc(C(F)F)ncc1NC(=O)N(c1ccccc1C(C)C)C1CCN(C(C)=O)CC1] | 461.2 [M + H]⁺ |
| 115 | 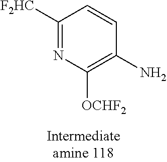 [SMILES: Nc1ccc(C(F)F)nc1OC(F)F] | 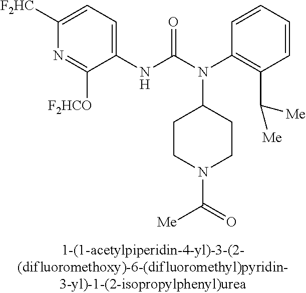 [SMILES: CC(=O)N1CCC(N(C(=O)Nc2ccc(C(F)F)nc2OC(F)F)c2ccccc2C(C)C)CC1] | 497.3 [M + H]⁺ |
| 116 | 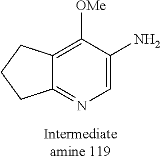 [SMILES: COc1c(N)cnc2c1CCC2] | 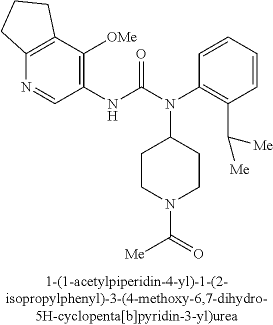 [SMILES: COc1c(NC(=O)N(c2ccccc2C(C)C)C2CCN(C(C)=O)CC2)cnc2c1CCC2] | 451.4 [M + H]⁺ |
| 217 | 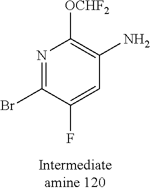 [SMILES: Nc1cc(F)c(Br)nc1OC(F)F] | 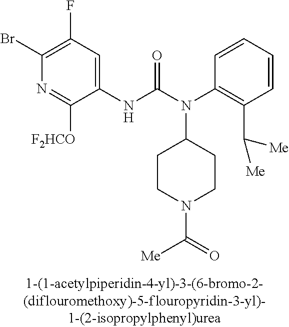 [SMILES: CC(=O)N1CCC(N(C(=O)Nc2cc(F)c(Br)nc2OC(F)F)c2ccccc2C(C)C)CC1] | 543.0, 545.0 [M + H]⁺ |
| 246 | 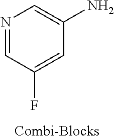 | 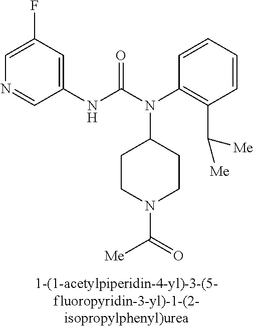 [SMILES: CC(=O)N1CCC(N(C(=O)Nc2cncc(F)c2)c2ccccc2C(C)C)CC1] | 399.9 [M + H]⁺ |

### Example 117: Preparation of 1-(1-acetylazetidin-3-yl)-3-(2-(difluoromethoxy)-4-methylphenyl)-1-(2-isopropylphenyl)urea

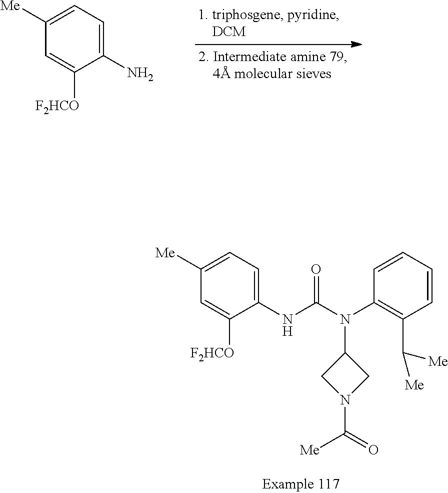 [SMILES: Cc1ccc(N)c(OC(F)F)c1 ; CC(=O)N1CC(N(C(=O)Nc2ccc(C)cc2OC(F)F)c2ccccc2C(C)C)C1]

In a dried, round-bottom flask equipped with a magnetic stirrer was combined 2-(difluoromethoxy)-4-methylaniline (1 equiv, Enamine) and pyridine (3 equiv, Sigma-Aldrich) in anhydrous dichloromethane (0.070 M). To this was then added triphosgene (0.5 equiv, Sigma-Aldrich) in one rapid portion at RT and the resulting solution was stirred at RT for 30 min. To this mixture was then added, at 0° C., Intermediate amine 79 (1 equiv) and freshly activated 4 Å molecular sieves. The resulting suspension was warmed to and stirred at RT for 16 h, before the reaction was quenched with water. The aqueous layer was separated and back extracted with DCM. The combined organic extracts were dried over Na₂SO₄, filtered, and the filtrate concentrated in vacuo. Purification of the crude product thus obtained by way of reverse-phase column chromatography (Cig, gradient elution: 95:5 (v/v) H₂O: MeCN→MeCN) afforded the title compound as a white solid (3100 yield). LCMS: m/z=432.2 [M+H]⁺; ¹H NMR (methanol-d₄): δ=7.82 (dd, J=8.4, 2.0 Hz, 1H), 7.61˜7.55 (in, 2H), 7.42˜7.31 (m, 2H), 7.01 (dd, J=8.4, 1.2 Hz, 1H), 6.92 (s, 1H), 6.55 (t, J=74.0 Hz, 1H), 4.94˜4.88 (m, 1H), 4.51˜4.47 (m, 1H), 4.27˜4.16 (m, 2H), 3.95˜3.81 (m, 1H), 3.32˜3.06 (m, 1H), 2.29 (s, 3H), 1.86 (s, 1.5H, rotamer A), 1.84 (s, 1.5H, rotamer B), 1.26˜1.23 (in, 6H).

The following examples were prepared in an analogous fashion to Example 117, but substituting 2-(difluoromethoxy)-4-methyl aniline with the requisite amine.

| | | | LCMS |
|---|---|---|---|
| Example | Starting Amine | Structure | (m/z) |
| 118 | 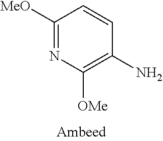 [SMILES: COc1ccc(N)c(OC)n1] | 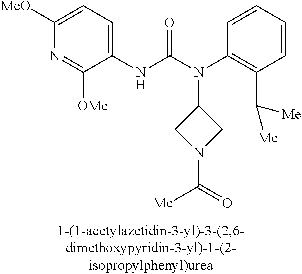 [SMILES: COc1ccc(NC(=O)N(c2ccccc2C(C)C)C2CN(C(C)=O)C2)c(OC)n1] | 413.2 [M + H]⁺ |
| 119 | 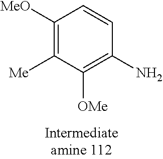 [SMILES: COc1ccc(N)c(OC)c1C] | 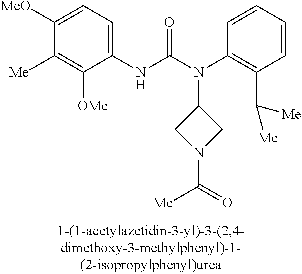 [SMILES: COc1ccc(NC(=O)N(c2ccccc2C(C)C)C2CN(C(C)=O)C2)c(OC)c1C] | 426.2 [M + H]⁺ |
| 120 | 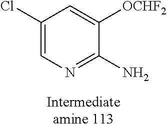 [SMILES: Nc1ncc(Cl)cc1OC(F)F] | 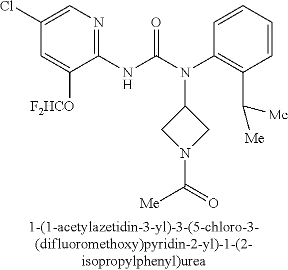 [SMILES: CC(=O)N1CC(N(C(=O)Nc2ncc(Cl)cc2OC(F)F)c2ccccc2C(C)C)C1] | 453.1, 455.1 [M + H]⁺ |

### Example 121: Preparation of 3-(2-(difluoromethoxy)-6-methoxypyridin-3-yl)-1-(2-isopropylphenyl)-1-(1-(oxetan-3-yl)piperidin-4-yl)urea

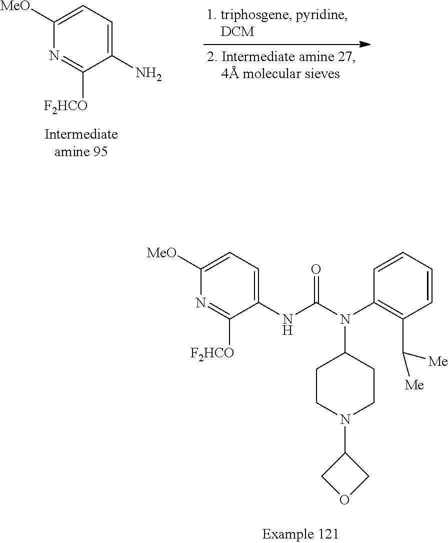 [SMILES: COc1ccc(N)c(OC(F)F)n1 ; COc1ccc(NC(=O)N(c2ccccc2C(C)C)C2CCN(C3COC3)CC2)c(OC(F)F)n1]

In a dried, round-bottom flask equipped with a magnetic stirrer was combined Intermediate amine 95 (1 equiv) and pyridine (3 equiv, Sigma-Aldrich) in anhydrous dichloromethane (0.13 M). To this was then added triphosgene (0.3 equiv, Sigma-Aldrich) in one rapid portion at RT and the resulting solution was stirred at RT for 30 min. To this mixture was then added, at 0° C., Intermediate amine 27 (1 equiv) and freshly activated 4 Å molecular sieves. The resulting suspension was warmed to and stirred at RT for 16 h, before the reaction was quenched with water. The aqueous layer was separated and back extracted with DCM. The combined organic extracts were dried over Na₂SO₄, filtered, and the filtrate concentrated in vacuo. Purification of the crude product thus obtained by way of reverse-phase column chromatography (C₁s, gradient elution: 95:5 (v/v) H₂O: MeCN→MeCN) afforded the title compound as a white solid (31% yield). LCMS: m/z=491.2 [M+H]⁺; ¹H NMR (methanol-d₄): δ=8.05 (d, J=8.4 Hz, 1H), 7.60˜7.12 (m, 5H), 6.53 (d, J=9.4 Hz, 1H), 4.65˜4.60 (m, 2H), 4.54˜4.48 (m, 2H), 4.38˜4.32 (m, 1H), 3.81 (s, 3H), 3.49˜3.42 (m, 1H), 3.23˜3.18 (m, 1H), 2.85˜2.78 (m, 2H), 2.12˜1.66 (m, 5H), 1.41˜1.28 (m, 4H), 1.21 (d, J=6.9 Hz, 3H).

The following example was prepared in an analogous fashion to Example 121, but substituting Intermediate amine 95 with the requisite amine.

| | | | LCMS |
|---|---|---|---|
| Example | Starting Amine | Structure | (m/z) |
| 122 | 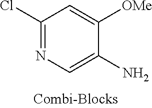 | 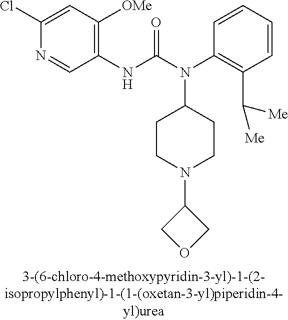 [SMILES: COc1cc(Cl)ncc1NC(=O)N(c1ccccc1C(C)C)C1CCN(C2COC2)CC1] | 459.2, 460.2 [M + H]⁺ |

### Example 123: Preparation of 1-(2-acetyl-2-azaspiro[3.3]heptan-6-yl)-3-(5-bromo-3-(difluoromethoxy)pyridin-2-yl)-1-(2-isopropylphenyl)urea

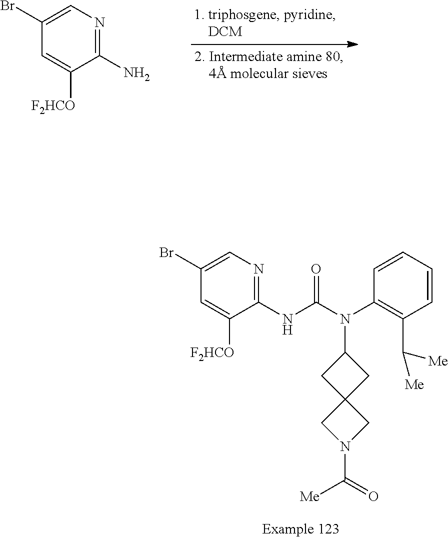 [SMILES: Nc1ncc(Br)cc1OC(F)F ; CC(=O)N1CC2(CC(N(C(=O)Nc3ncc(Br)cc3OC(F)F)c3ccccc3C(C)C)C2)C1]

In a dried, round-bottom flask equipped with a magnetic stirrer was combined 5-bromo-3-(difluoromethoxy)pyridin-2-amine (1 equiv, Ambeed) and pyridine (3 equiv, Sigma-Aldrich) in anhydrous dichloromethane (0.045 M). To this was then added triphosgene (0.5 equiv, Sigma-Aldrich) in one rapid portion at RT and the resulting solution was stirred at RT for 30 min. To this mixture was then added, at 0° C., Intermediate amine 80 (1 equiv) and freshly activated 4 Å molecular sieves. The resulting suspension was warmed to and stirred at RT for 2 h, before the reaction was quenched with water. The aqueous layer was separated and back extracted with DCM. The combined organic extracts were dried over Na₂SO₄, filtered, and the filtrate concentrated in vacuo. Purification of the crude product thus obtained by way of reverse-phase column chromatography (C₁₈, gradient elution: 95:5 (v/v) H₂O: MeCN→MeCN) afforded the title compound as a white solid (33% yield). LCMS: m/z=537.2, 539.2 [M+H]⁺; ¹H NMR (methanol-d₄): δ=8.26 (d, J=2.0 Hz, 1H), 7.77 (d, J=2.0 Hz, 1H), 7.58˜7.51 (m, 2H), 7.41˜7.37 (m, 1H), 7.26 (d, J=7.6 Hz, 1H), 6.78 (t, J=72.8 Hz, 1H), 4.86˜4.69 (m, 1H), 4.28 (s, 1H), 4.06˜3.98 (m, 2H), 3.78˜3.72 (m, 1H), 3.11˜3.03 (m, 1H), 2.61˜2.56 (m, 1H), 2.47˜2.43 (m, 1H), 2.32˜2.27 (m, 1H), 2.08˜1.95 (m, 1H), 1.84 (s, 1.5H, rotamer A), 1.80 (s, 1.5H, rotamer B), 1.29˜1.21 (m, 6H).

The following example was prepared in an analogous fashion to Example 123, but substituting 5-bromo-3-(difluoromethoxy)pyridin-2-amine with the requisite amine.

| | | | LCMS |
|---|---|---|---|
| Example | Starting Amine | Structure | (m/z) |
| 124 | 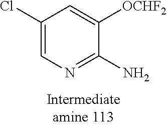 [SMILES: Nc1ncc(Cl)cc1OC(F)F] | 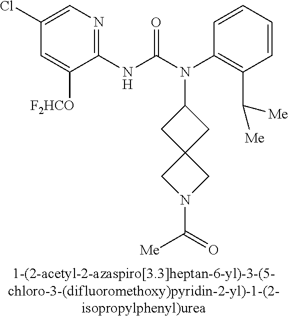 [SMILES: CC(=O)N1CC2(CC(N(C(=O)Nc3ncc(Cl)cc3OC(F)F)c3ccccc3C(C)C)C2)C1] | 493.2, 495.2 [M + H]⁺ |

### Example 125: Preparation of 1-(2-acetyl-2-azaspiro[3.3]heptan-6-yl)-3-(2-(difluoromethoxy)-4-methylphenyl)-1-(2-isopropylphenyl)urea

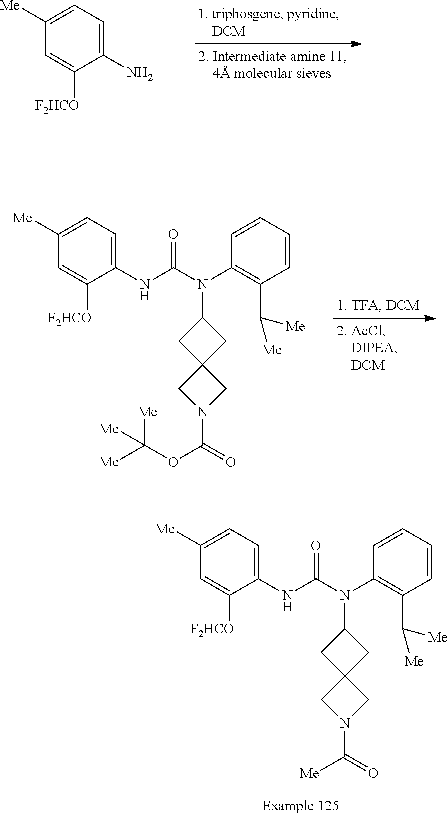 [SMILES: Cc1ccc(N)c(OC(F)F)c1 ; Cc1ccc(NC(=O)N(c2ccccc2C(C)C)C2CC3(C2)CN(C(=O)OC(C)(C)C)C3)c(OC(F)F)c1 ; CC(=O)N1CC2(CC(N(C(=O)Nc3ccc(C)cc3OC(F)F)c3ccccc3C(C)C)C2)C1]

Step 1: In a dried, round-bottom flask equipped with a magnetic stirrer was combined 2-(difluoromethoxy)-4-methylaniline (1 equiv, Enamine) and pyridine (3 equiv, Sigma-Aldrich) in anhydrous dichloromethane (0.39 M). To this was then added triphosgene (0.35 equiv, Sigma-Aldrich) in one rapid portion at RT and the resulting solution was stirred at RT for 30 min. To this mixture was then added, at 0° C., Intermediate amine 11 (1 equiv) and freshly activated 4 Å molecular sieves. The resulting suspension was warmed to and stirred at RT for 16 h, before the reaction was quenched with water. The aqueous layer was separated and back extracted with DCM. The combined organic extracts were dried over Na₂SO₄, filtered, and the filtrate concentrated in vacuo. Purification of the crude product thus obtained by way of column chromatography (SiO₂, gradient elution: Hex→EtOAc) afforded tert-butyl 6-(3-(2-(difluoromethoxy)-4-methylphenyl)-1-(2-isopropylphenyl)ureido)-2-azaspiro[3.3]heptane-2-carboxylate as an orange solid (58% yield).

Step 2: In a round-bottom flask equipped with a magnetic stirrer was dissolved tert-butyl 6-(3-(2-(difluoromethoxy)-4-methylphenyl)-1-(2-isopropylphenyl)ureido)-2-azaspiro[3.3]heptane-2-carboxylate (1 equiv) from the previous step in dichloromethane (0.075 M). To this solution was then added trifluoroacetic acid (150 equiv, Sigma-Aldrich) and the resulting reaction mixture was stirred at RT for 1 h. The volatiles were then removed in vacuo via sequential azeotropic distillation with toluene and heptane. The residue thus obtained was then partitioned between EtOAc and saturated aq. NaHCO₃. The aqueous layer was separated and back extracted with EtOAc. The combined organic extracts were washed further with water and brine, dried over Na₂SO₄, filtered, and the filtrate concentrated in vacuo. The crude 3-(2-(difluoromethoxy)-4-methylphenyl)-1-(2-isopropylphenyl)-1-(2-azaspiro[3.3]heptan-6-yl)urea thus obtained was then re-taken up in dichloromethane (0.058 M), and added sequentially N,N-diisopropylethylamine (3 equiv, Sigma-Aldrich) and acetyl chloride (1.5 equiv, Sigma-Aldrich). After 1 h of stirring at RT, the reaction was quenched with water and extracted with DCM. The combined organic extracts were washed further with brine, dried over MgSO₄, filtered, and the filtrate concentrated in vacuo. Purification of the crude product thus obtained by way of reverse-phase column chromatography (C₁₈, gradient elution: 95:5 (v/v) H₂O: MeCN→MeCN) afforded the title compound as a white solid (43% yield). LCMS: m/z=472.2 [M+H]⁺; ¹H NMR (methanol-d₄): δ=7.87 (d, J=8.1 Hz, 1H), 7.59˜7.49 (m, 2H), 7.43˜7.38 (m, 1H), 7.25 (d, J=7.8 Hz, 1H), 6.95 (dd, J=8.4, 1.2 Hz, 1H), 6.90 (s, 1H), 6.52 (t, J=73.8 Hz, 1H), 4.87˜4.68 (m, 1H), 4.29 (s, 1H), 4.05˜3.95 (m, 2H), 3.79˜3.72 (m, 1H), 3.09˜3.02 (m, 1H), 2.62˜2.38 (m, 2H), 2.28˜2.01 (m, 4H), 2.01˜1.94 (m, 1H), 1.85 (s, 1.5H, rotamer A), 1.81 (s, 1.5H, rotamer B), 1.28 (d, J=6.9 Hz, 3H), 1.18 (d, J=6.9 Hz, 3H).

The following example was prepared in an analogous fashion to Example 125, but substituting acetyl chloride in step 2 with the requisite electrophile.

| | Starting | | LCMS |
|---|---|---|---|
| Example | Electrophile | Structure | (m/z) |
| 126 | 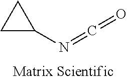 | 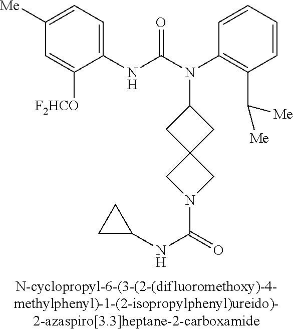 [SMILES: Cc1ccc(NC(=O)N(c2ccccc2C(C)C)C2CC3(C2)CN(C(=O)NC2CC2)C3)c(OC(F)F)c1] | 513.3 [M + H]⁺ |

### Example 127: 3-(3-(2-(difluoromethoxy)-6-methylpyridin-3-yl)-1-(2-isopropylphenyl)ureido)azetidine-1-sulfonamide

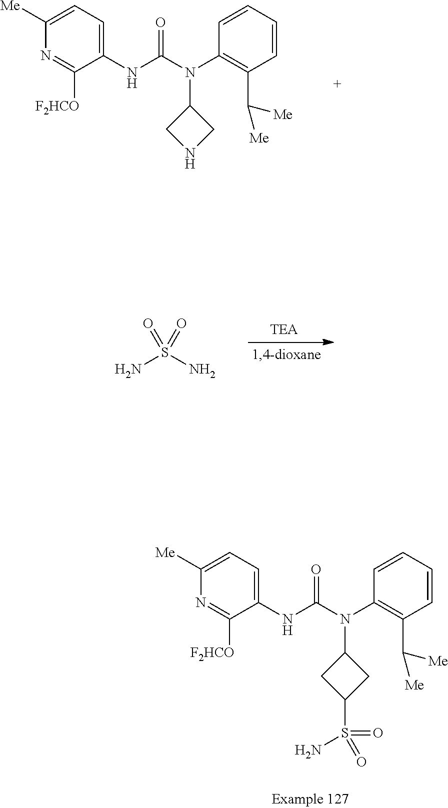 [SMILES: Cc1ccc(NC(=O)N(c2ccccc2C(C)C)C2CNC2)c(OC(F)F)n1 ; NS(N)(=O)=O ; Cc1ccc(NC(=O)N(c2ccccc2C(C)C)C2CC(S(N)(=O)=O)C2)c(OC(F)F)n1]

In a round-bottom flask equipped with a magnetic stirrer was combined 1-(azetidin-3-yl)-3-(2-(difluoromethoxy)-6-methylpyridin-3-yl)-1-(2-isopropylphenyl)urea (1 equiv, Example 67, step 1) and triethylamine (3 equiv, Sigma-Aldrich) in 1,4-dioxane (0.045 M). To this was then added sulfamide (1.1 equiv, Ambeed) and the resulting mixture was heated at 100° C. for 16 h. The volatiles were then removed in vacuo and the crude product thus obtained was purified by way of reverse-phase column chromatography (C₁₈, gradient elution: 9:1 (v/v) H₂O: MeCN→1:6 (v/v) H₂O: MeCN) to afford the title compound as a white solid (54% yield). LCMS: m/z=470.2 [M+H]⁺; ¹H NMR (methanol-d₄): δ=8.23 (d, J=8.1 Hz, 1H), 7.64˜7.13 (m, 5H), 7.00 (d, J=8.1 Hz, 1H), 4.99˜4.82 (m, 1H), 4.01˜3.64 (m, 4H), 3.10 (sept, J=6.9 Hz, 1H), 2.38 (s, 3H), 1.28 (d, J=6.9 Hz, 3H), 1.14 (d, J=6.9 Hz, 3H).

The following example was prepared in an analogous fashion to Example 127, but substituting 1-(azetidin-3-yl)-3-(2-(difluoromethoxy)-6-methylpyridin-3-yl)-1-(2-isopropylphenyl)urea with the requisite amine.

| | | | LCMS |
|---|---|---|---|
| Example | Starting Amine | Structure | (m/z) |
| 128 | 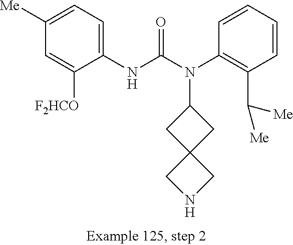 [SMILES: Cc1ccc(NC(=O)N(c2ccccc2C(C)C)C2CC3(CNC3)C2)c(OC(F)F)c1] | 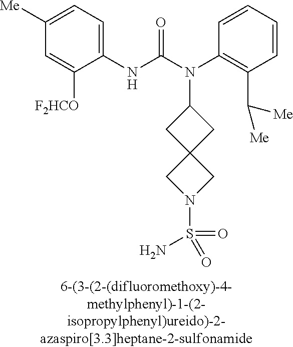 [SMILES: Cc1ccc(NC(=O)N(c2ccccc2C(C)C)C2CC3(C2)CN(S(N)(=O)=O)C3)c(OC(F)F)c1] | 509.2 [M + H]⁺ |

### Example 129: Preparation of 1-(1-acetylpiperidin-4-yl)-3-(5-bromo-3-(difluoromethoxy)pyridin-2-yl)-1-(2-isopropylphenyl)urea

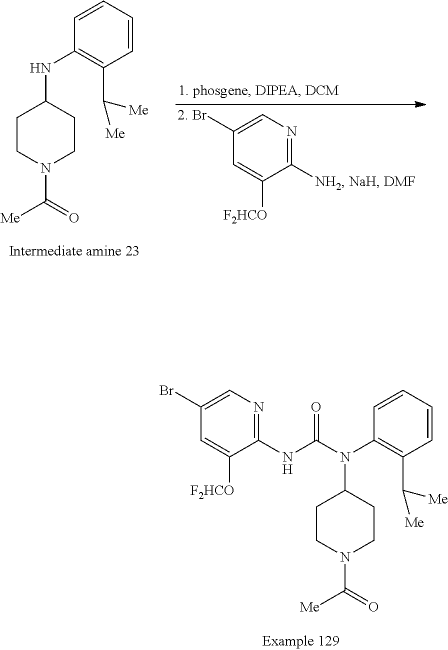

In a dried, round-bottom flask equipped with a magnetic stirrer was combined Intermediate amine 23 (1 equiv) and N,N-diisopropylethylamine (2 equiv, Sigma-Aldrich) in anhydrous dichloromethane (0.1 M). To this was then added phosgene (15% w/w solution in toluene, 1.3 equiv, Sigma-Aldrich) dropwise at RT and the resulting solution was stirred at RT for 15 min. The volatiles were then removed in vacuo and the resulting residue was partitioned between water and dichloromethane. The organic layer was separated, washed further with brine, dried over MgSO₄, and filtered. Concentration of the filtrate thus obtained in vacuo furnished crude (1-acetylpiperidin-4-yl)(2-isopropylphenyl)carbamic chloride as a tan solid. In a separate dried, round-bottom flask equipped with a magnetic stirrer was dissolved 5-bromo-3-(difluoromethoxy)pyridin-2-amine (2 equiv, AstaTech) in DMF (0.13 M). To this was then added sodium hydride (60% w/w dispersion in paraffin oil, 3 equiv, Sigma-Aldrich) in one rapid portion and the resulting mixture was stirred at RT for 10 min to afford a yellow-orange solution. Finally, a DMF solution (0.1 M) of crude (1-acetylpiperidin-4-yl)(2-isopropylphenyl)carbamic chloride prepared above was added dropwise over a period of 5 min. The reaction mixture thus obtained was stirred at RT for another 30 min before it was carefully quenched with saturated aq. NH₄Cl and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: 9:1 (v/v) Hex: EtOAc+10% MeOH→EtOAc+10% MeOH) afforded the title compound as a white foam (61% yield). LCMS: m/z=524.7, 526.7 [M+H]⁺; ¹H NMR (CDCl₃): δ=8.28˜8.27 (m, 1H), 7.56˜7.25 (m, 4H), 7.18˜7.08 (m, 1H), 6.62˜6.13 (m, 2H), 4.75˜4.51 (m, 2H), 3.75˜3.66 (m, 1H), 3.29˜3.09 (m, 2H), 2.66˜2.52 (m, 1H), 2.24˜1.48 (m, 6H), 1.32˜1.15 (m, 7H).

The following examples were prepared in an analogous fashion to Example 129, but substituting 5-bromo-3-(difluoromethoxy)pyridin-2-amine with the requisite amine.

| | | | LCMS |
|---|---|---|---|
| Example | Starting Amine | Structure | (m/z) |
| 130 | 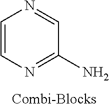 | 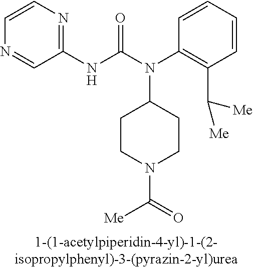 [SMILES: CC(=O)N1CCC(N(C(=O)Nc2cnccn2)c2ccccc2C(C)C)CC1] | 382.2 [M + H]⁺ |
| 131 | 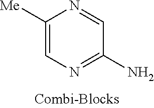 | 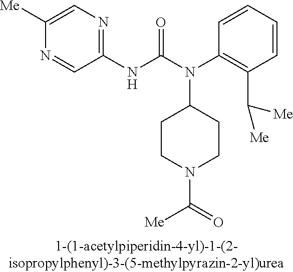 [SMILES: CC(=O)N1CCC(N(C(=O)Nc2cnc(C)cn2)c2ccccc2C(C)C)CC1] | 396.2 [M + H]⁺ |
| 132 | 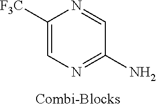 [SMILES: Nc1cnc(C(F)(F)F)cn1] | 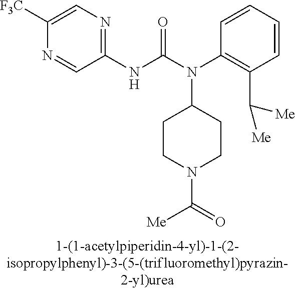 [SMILES: CC(=O)N1CCC(N(C(=O)Nc2cnc(C(F)(F)F)cn2)c2ccccc2C(C)C)CC1] | 450.1 [M + H]⁺ |
| 133 | 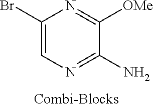 | 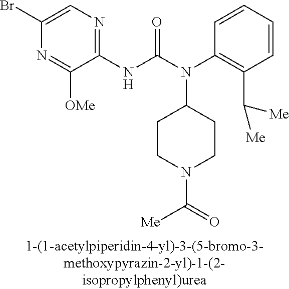 [SMILES: COc1nc(Br)cnc1NC(=O)N(c1ccccc1C(C)C)C1CCN(C(C)=O)CC1] | 490.0, 492.0 [M + H]⁺ |
| 134 | 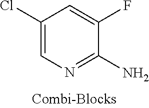 | 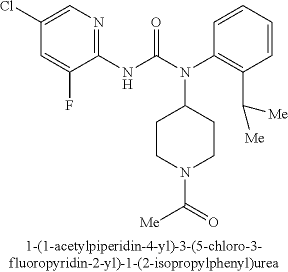 [SMILES: CC(=O)N1CCC(N(C(=O)Nc2ncc(Cl)cc2F)c2ccccc2C(C)C)CC1] | 433.1 435.1 [M + H]⁺ |
| 135 | 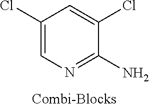 | 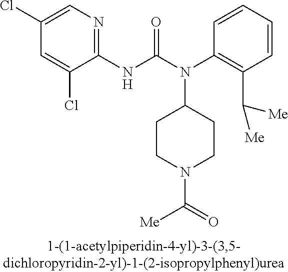 [SMILES: CC(=O)N1CCC(N(C(=O)Nc2ncc(Cl)cc2Cl)c2ccccc2C(C)C)CC1] | 449.0, 451.0 [M + H]⁺ |
| 136 | 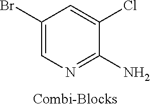 | 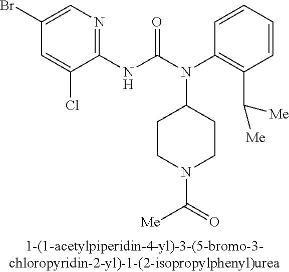 [SMILES: CC(=O)N1CCC(N(C(=O)Nc2ncc(Br)cc2Cl)c2ccccc2C(C)C)CC1] | 493.0, 495.0, 497.0 [M + H]⁺ |
| 137 | 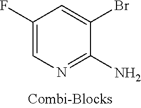 | 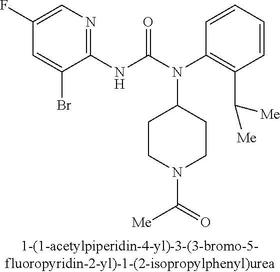 [SMILES: CC(=O)N1CCC(N(C(=O)Nc2ncc(F)cc2Br)c2ccccc2C(C)C)CC1] | 477.0, 479.0 [M + H]⁺ |
| 138 | 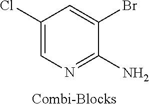 | 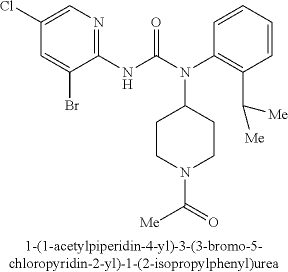 [SMILES: CC(=O)N1CCC(N(C(=O)Nc2ncc(Cl)cc2Br)c2ccccc2C(C)C)CC1] | 493.0, 495.0, 497.0 [M + H]⁺ |
| 139 | 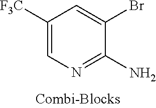 [SMILES: Nc1ncc(C(F)(F)F)cc1Br] | 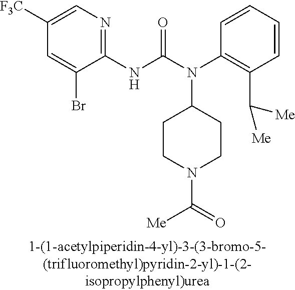 [SMILES: CC(=O)N1CCC(N(C(=O)Nc2ncc(C(F)(F)F)cc2Br)c2ccccc2C(C)C)CC1] | 527.0, 529.0 [M + H]⁺ |
| 140 | 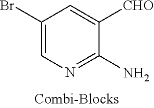 | 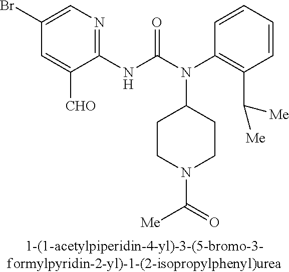 [SMILES: CC(=O)N1CCC(N(C(=O)Nc2ncc(Br)cc2C=O)c2ccccc2C(C)C)CC1] | 487.0, 489.0 [M + H]⁺ |
| 141 | 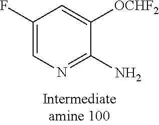 [SMILES: Nc1ncc(F)cc1OC(F)F] | 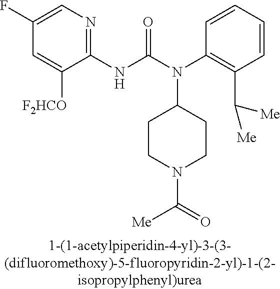 [SMILES: CC(=O)N1CCC(N(C(=O)Nc2ncc(F)cc2OC(F)F)c2ccccc2C(C)C)CC1] | 465.0 [M + H]⁺ |
| 226 | 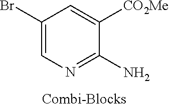 [SMILES: COC(=O)c1cc(Br)cnc1N] | 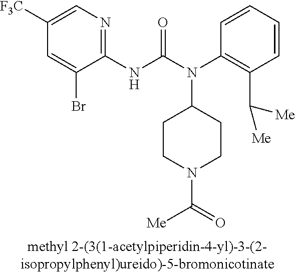 [SMILES: CC(=O)N1CCC(N(C(=O)Nc2ncc(C(F)(F)F)cc2Br)c2ccccc2C(C)C)CC1] | 517.0, 519.0 [M + H]⁺ |
| 230 | 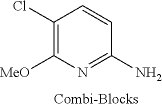 | 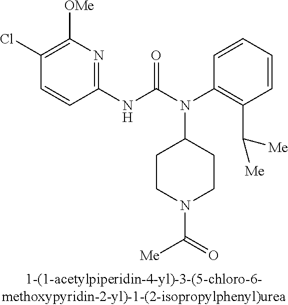 [SMILES: COc1nc(NC(=O)N(c2ccccc2C(C)C)C2CCN(C(C)=O)CC2)ccc1Cl] | 445.1, 447.1 [M + H]⁺ |

The following examples were prepared in an analogous fashion to Example 129, but substituting Intermediate amine 23 with the requisite amine.

| | | | LCMS |
|---|---|---|---|
| Example | Starting Amine | Structure | (m/z) |
| 142 | 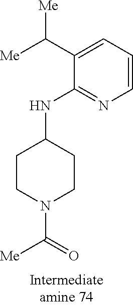 [SMILES: CC(=O)N1CCC(Nc2ncccc2C(C)C)CC1] | 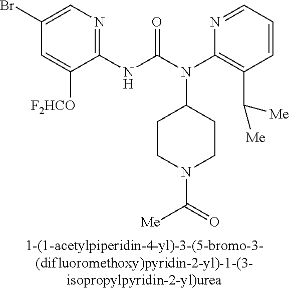 [SMILES: CC(=O)N1CCC(N(C(=O)Nc2ncc(Br)cc2OC(F)F)c2ncccc2C(C)C)CC1] | 526.0, 528.0 [M + H]⁺ |

### Example 143: Preparation of 1-(1-acetylpiperidin-4-yl)-1-(2-isopropylphenyl)-3-(5-methylpyridin-2-yl)urea

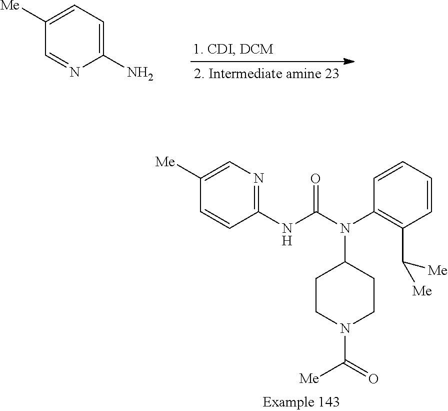 [SMILES: Cc1ccc(N)nc1 ; CC(=O)N1CCC(N(C(=O)Nc2ccc(C)cn2)c2ccccc2C(C)C)CC1]

In a dried, round-bottom flask equipped with a magnetic stirrer was dissolved 5-methylpyridin-2-amine (1 equiv, Enamine) in anhydrous dichloromethane (0.095 M). To this was then added 1,1′-carbonyldiimidazole (1.5 equiv, Sigma-Aldrich) portionwise at 0° C. over 5 min, and the resulting solution was stirred at 0° C. for another 30 min. To this mixture was then added, at 0° C., Intermediate amine 23 (1 equiv). The resulting mixture was warmed to and stirred at RT for 24 h, before the reaction was quenched with water. The aqueous layer was separated and back extracted with DCM. The combined organic extracts were dried over Na₂SO₄, filtered, and the filtrate concentrated in vacuo. Purification of the crude product thus obtained by way of reverse-phase column chromatography (C₁s, gradient elution: 95:5 (v/v) H₂O: MeCN→MeCN) afforded the title compound as a white solid (57% yield). LCMS: m/z=395.3 [M+H]⁺; ¹H NMR (methanol-d₄): δ=7.93˜7.87 (m, 2H), 7.61˜7.48 (m, 3H), 7.40˜7.34 (m, 1H), 7.26 (d, J=8.1 Hz, 1H), 4.62˜4.52 (m, 2H), 4.01˜3.96 (m, 1H), 3.27˜3.05 (m, 2H), 2.73˜2.65 (m, 1H), 2.23 (s, 3H), 2.22˜2.16 (m, 1H), 2.06 (s, 1.5H, rotamer A), 2.02 (s, 1.5H, rotamer B), 1.87˜1.39 (m, 2H), 1.32˜1.08 (m, 7H).

### Example 144: Preparation of trans-4-(3-(2-(difluoromethoxy)-6-methoxypyridin-3-yl)-1-(2-isopropylphenyl)ureido)cyclohexane-1-carboxylic acid

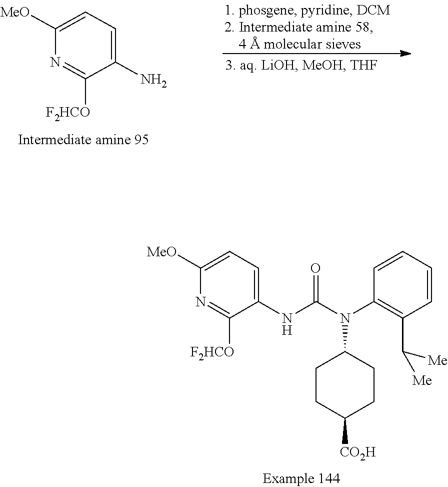 [SMILES: COc1ccc(N)c(OC(F)F)n1 ; COc1ccc(NC(=O)N(c2ccccc2C(C)C)[C@H]2CC[C@H](C(=O)O)CC2)c(OC(F)F)n1]

Step 1: In a dried, round-bottom flask equipped with a magnetic stirrer was combined Intermediate amine 95 (1 equiv) and pyridine (3 equiv, Sigma-Aldrich) in anhydrous dichloromethane (0.086 M). To this was then added phosgene (15% w/w solution in toluene, 1.5 equiv, Sigma-Aldrich) dropwise at RT and the resulting solution was stirred at RT for 15 min. The volatiles were then removed in vacuo and the crude (2-(difluoromethoxy)-6-methoxypyridin-3-yl)carbamic chloride thus obtained was re-taken up in anhydrous dichloromethane (0.12 M). This solution was then added dropwise at RT to another dichloromethane suspension (0.12 M) of Intermediate amine 58 (1 equiv), pyridine (3 equiv, Sigma-Aldrich), and freshly activated 4 Å molecular sieves. The resulting mixture was stirred at RT for 24 h before the reaction was quenched with water. The aqueous layer was separated and back extracted with EtOAc. The combined organic extracts were dried over MgSO₄, filtered, and the filtrate concentrated in vacuo. Purification of the crude product thus obtained by way of column chromatography (SiO₂, gradient elution: Hex→7:3 (v/v) Hex: EtOAc) afforded trans-ethyl 4-(3-(2-(difluoromethoxy)-6-methoxypyridin-3-yl)-1-(2-isopropylphenyl)ureido)cyclohexane-1-carboxylate as a white foam (81% yield).

Step 2: In a round-bottom flask equipped with a magnetic stirrer was dissolved trans-ethyl 4-(3-(2-(difluoromethoxy)-6-methoxypyridin-3-yl)-1-(2-isopropylphenyl)ureido)cyclohexane-1-carboxylate (1 equiv) from the previous step in a 1:1 (v/v) solution of THE and methanol (0.11 M). To this colorless solution was then added lithium hydroxide (1 M solution in water, 5 equiv) and the resulting mixture was heated at 45° C. for 3 h. Upon cooling to RT, the reaction mixture was carefully quenched with HCl (1 M solution in water, 5 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded the title compound as a white foam (92% yield). LCMS: m/z=478.1 [M+H]⁺; ¹H NMR (DMSO-d₆): δ=8.08 (d, J=8.7 Hz, 1H), 7.56˜7.49 (m, 2H), 7.36 (t, J=72.9 Hz, 1H), 7.35 (td, J=7.2, 1.8 Hz, 1H), 7.25 (dd, J=8.1, 1.2 Hz, 1H), 6.54 (d, J=8.7 Hz, 1H), 6.29 (s, 1H), 4.30˜4.21 (m, 1H), 3.82 (s, 3H), 3.22 (sept, J=6.9 Hz, 1H), 2.24˜1.85 (m, 5H), 1.62˜1.46 (m, 3H), 1.29 (d, J=6.9 Hz, 3H), 1.22˜1.05 (m, 4H).

The following examples were prepared in an analogous fashion to Example 144, but substituting Intermediate amine 58 with the requisite amine. In Example 277, step 2 was obviated. In Example 284, Example 286 and Example 340, phosgene (1.5 equiv) in step 1 was also substituted with triphosgene (0.5 equiv, Sigma-Aldrich).

| | | | LCMS |
|---|---|---|---|
| Example | Starting Amine | Structure | (m/z) |
| 145 | 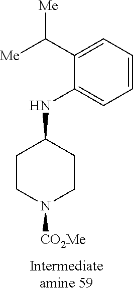 | 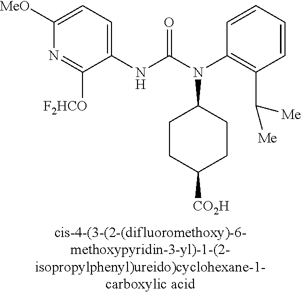 [SMILES: COc1ccc(NC(=O)N(c2ccccc2C(C)C)[C@H]2CC[C@@H](C(=O)O)CC2)c(OC(F)F)n1] | 478.2 [M + H]⁺ |
| 146 | 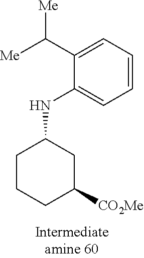 [SMILES: COC(=O)[C@H]1CCC[C@H](Nc2ccccc2C(C)C)C1] | 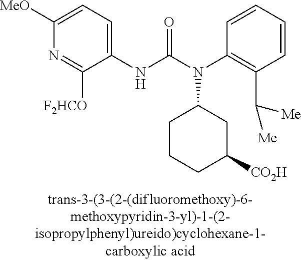 [SMILES: COc1ccc(NC(=O)N(c2ccccc2C(C)C)[C@H]2CCC[C@H](C(=O)O)C2)c(OC(F)F)n1] | 478.2 [M + H]⁺ |
| 147 | 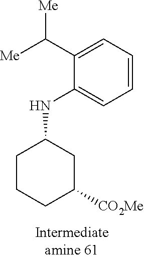 [SMILES: COC(=O)[C@@H]1CCC[C@H](Nc2ccccc2C(C)C)C1] | 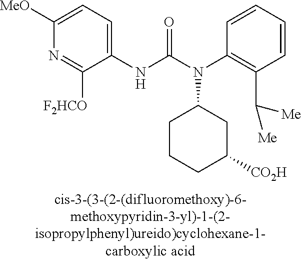 [SMILES: COc1ccc(NC(=O)N(c2ccccc2C(C)C)[C@H]2CCC[C@@H](C(=O)O)C2)c(OC(F)F)n1] | 478.3 [M + H]⁺ |
| 148 | 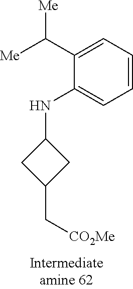 [SMILES: COC(=O)CC1CC(Nc2ccccc2C(C)C)C1] | 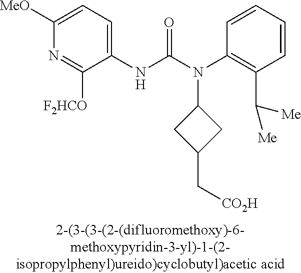 [SMILES: COc1ccc(NC(=O)N(c2ccccc2C(C)C)C2CC(CC(=O)O)C2)c(OC(F)F)n1] | 464.0 [M + H]⁺ |
| 149 | 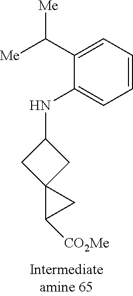 [SMILES: COC(=O)C1CC12CC(Nc1ccccc1C(C)C)C2] | 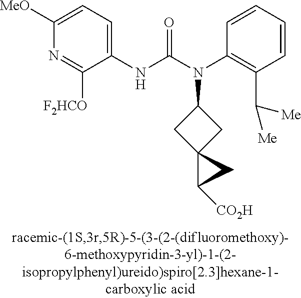 [SMILES: COc1ccc(NC(=O)N(c2ccccc2C(C)C)[C@H]2C[C@@]3(C[C@@H]3C(=O)O)C2)c(OC(F)F)n1] | 476.2 [M + H]⁺ |
| 150 | 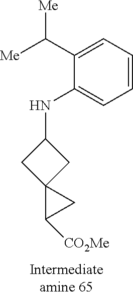 [SMILES: COC(=O)C1CC12CC(Nc1ccccc1C(C)C)C2] | 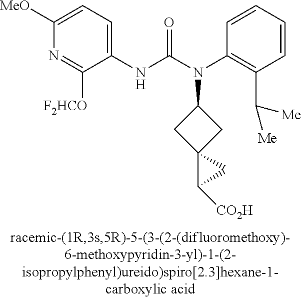 [SMILES: COc1ccc(NC(=O)N(c2ccccc2C(C)C)[C@H]2C[C@]3(C[C@H]3C(=O)O)C2)c(OC(F)F)n1] | 476.2 [M + H]⁺ |
| 151 | 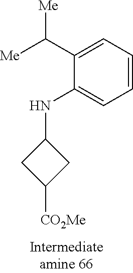 [SMILES: COC(=O)C1CC(Nc2ccccc2C(C)C)C1] | 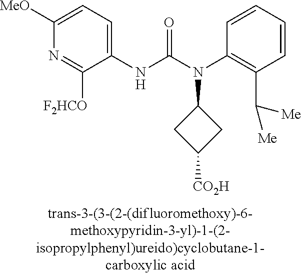 [SMILES: COc1ccc(NC(=O)N(c2ccccc2C(C)C)[C@H]2C[C@H](C(=O)O)C2)c(OC(F)F)n1] | 450.2 [M + H]⁺ |
| 152 | 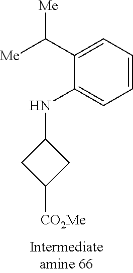 [SMILES: COC(=O)C1CC(Nc2ccccc2C(C)C)C1] | 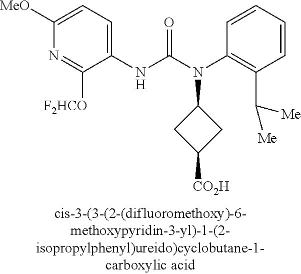 [SMILES: COc1ccc(NC(=O)N(c2ccccc2C(C)C)[C@H]2C[C@@H](C(=O)O)C2)c(OC(F)F)n1] | 450.2 [M + H]⁺ |
| 153 | 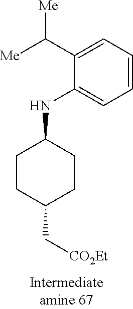 [SMILES: CCOC(=O)C[C@H]1CC[C@H](Nc2ccccc2C(C)C)CC1] | 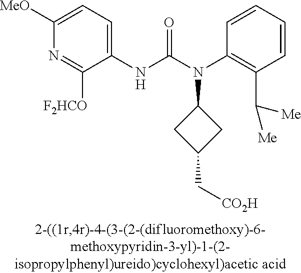 [SMILES: COc1ccc(NC(=O)N(c2ccccc2C(C)C)[C@H]2C[C@H](CC(=O)O)C2)c(OC(F)F)n1] | 492.0 [M + H]⁺ |
| 154 | 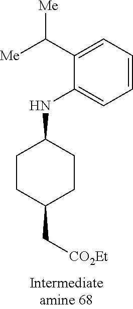 [SMILES: CCOC(=O)C[C@H]1CC[C@@H](Nc2ccccc2C(C)C)CC1] | 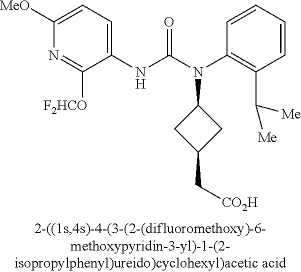 [SMILES: COc1ccc(NC(=O)N(c2ccccc2C(C)C)[C@H]2C[C@@H](CC(=O)O)C2)c(OC(F)F)n1] | 492.0 [M + H]⁺ |
| 233 | 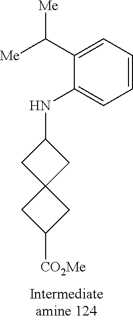 [SMILES: COC(=O)C1CC2(CC(Nc3ccccc3C(C)C)C2)C1] | 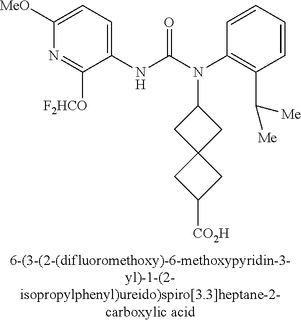 [SMILES: COc1ccc(NC(=O)N(c2ccccc2C(C)C)C2CC3(CC(C(=O)O)C3)C2)c(OC(F)F)n1] | 489.8 [M + H]⁺ |
| 234 | 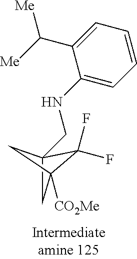 [SMILES: COC(=O)C12CC(CNc3ccccc3C(C)C)(C1)C2(F)F] | 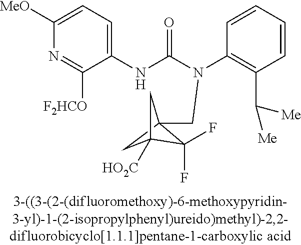 [SMILES: COc1ccc(NC(=O)N(CC23CC(C(=O)O)(C2)C3(F)F)c2ccccc2C(C)C)c(OC(F)F)n1] | 511.8 [M + H]⁺ |
| 274 | 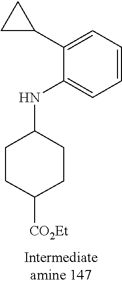 [SMILES: CCOC(=O)C1CCC(Nc2ccccc2C2CC2)CC1] | 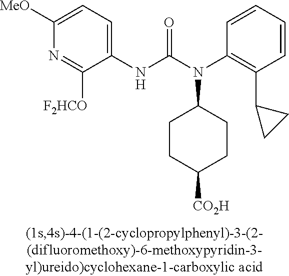 [SMILES: COc1ccc(NC(=O)N(c2ccccc2C2CC2)[C@H]2CC[C@@H](C(=O)O)CC2)c(OC(F)F)n1] | 476.1 [M + H]⁺ |
| 275 | 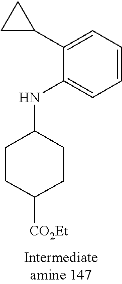 [SMILES: CCOC(=O)C1CCC(Nc2ccccc2C2CC2)CC1] | 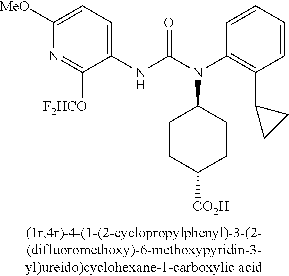 [SMILES: COc1ccc(NC(=O)N(c2ccccc2C2CC2)[C@H]2CC[C@H](C(=O)O)CC2)c(OC(F)F)n1] | 476.1 [M + H]⁺ |
| 277 | 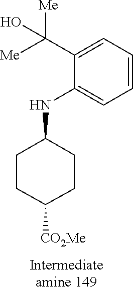 [SMILES: COC(=O)[C@H]1CC[C@H](Nc2ccccc2C(C)(C)O)CC1] | 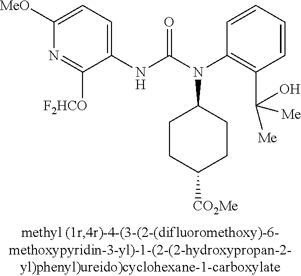 [SMILES: COc1ccc(NC(=O)N(c2ccccc2C(C)(C)O)[C@H]2CC[C@H](C(=O)OC)CC2)c(OC(F)F)n1] | 490.0 [M − H₂O + H]⁺ |
| 284 | 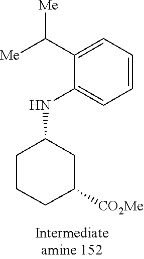 [SMILES: COC(=O)[C@@H]1CCC[C@H](Nc2ccccc2C(C)C)C1] | 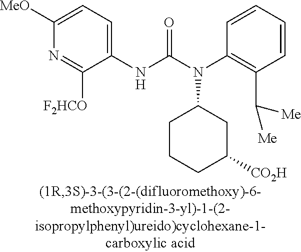 [SMILES: COc1ccc(NC(=O)N(c2ccccc2C(C)C)[C@H]2CCC[C@@H](C(=O)O)C2)c(OC(F)F)n1] | 478.2 [M + H]⁺ |
| 286 | 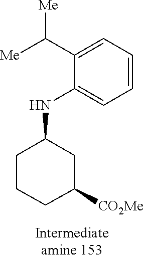 [SMILES: COC(=O)[C@H]1CCC[C@@H](Nc2ccccc2C(C)C)C1] | 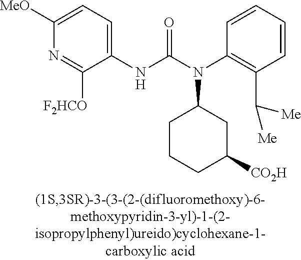 [SMILES: COc1ccc(NC(=O)N(c2ccccc2C(C)C)[C@@H]2CCC[C@H](C(=O)O)C2)c(OC(F)F)n1] | 478.2 [M + H]⁺ |
| 340 | 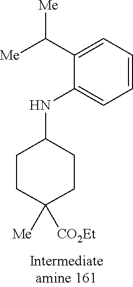 [SMILES: CCOC(=O)C1(C)CCC(Nc2ccccc2C(C)C)CC1] | 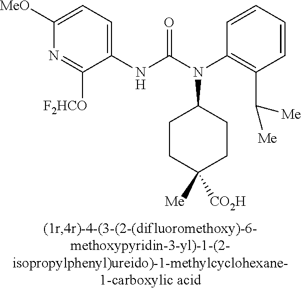 [SMILES: COc1ccc(NC(=O)N(c2ccccc2C(C)C)[C@H]2CC[C@](C)(C(=O)O)CC2)c(OC(F)F)n1] | 492.3 [M + H]⁺ |

The following examples were prepared in an analogous fashion to Example 144, but substituting Intermediate amine 95 with the requisite amine. In Example 157, Example 158, Example 288, Example 289, Example 294, Example 295, Example 296, Example 298, Example 299, Example 315, Example 316, Example 317, Example 318, Example 327, Example 328, Example 333, Example 334, Example 335, Example 336, Example 338, and Example 339, phosgene (1.5 equiv) was also substituted with triphosgene (0.4 equiv, Sigma-Aldrich), and 4-(dimethylamino)pyri dine (0.2 equiv, Sigma-Aldrich) was also included as an additive in step 1. Furthermore, in Example 298, Example 315, Example 316, Example 327, Example 328, and Example 339, the reaction was carried out at reflux for step 1. In Example 231, step 2 was obviated.

| | | | LCMS |
|---|---|---|---|
| Example | Starting Amine | Structure | (m/z) |
| 155 | 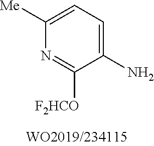 [SMILES: Cc1ccc(N)c(OC(F)F)n1] | 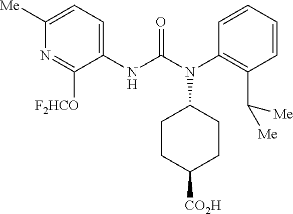 [SMILES: Cc1ccc(NC(=O)N(c2ccccc2C(C)C)[C@H]2CC[C@H](C(=O)O)CC2)c(OC(F)F)n1] | 462.2 [M + H]⁺ |
| | | trans-4-(3-(2-(difluoromethoxy)-6- | |
| | | methylpyridin-3-yl)-1-(2- | |
| | | isopropylphenyl)ureido)cyclohexane-1- | |
| | | carboxylic acid | |
| 156 | 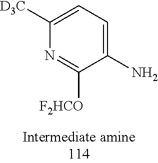 [SMILES: [2H]C([2H])([2H])c1ccc(N)c(OC(F)F)n1] | 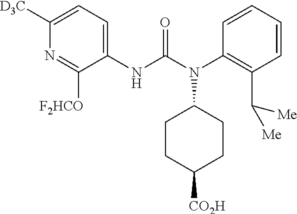 [SMILES: [2H]C([2H])([2H])c1ccc(NC(=O)N(c2ccccc2C(C)C)[C@H]2CC[C@H](C(=O)O)CC2)c(OC(F)F)n1] | 465.1 [M + H]⁺ |
| | | trans-4-(3-(2-(difluoromethoxy)-6-(methyl- | |
| | | d₃)pyridin-3-yl)-1-(2- | |
| | | isopropylphenyl)ureido)cyclohexane-1- | |
| | | carboxylic acid | |
| 157 | 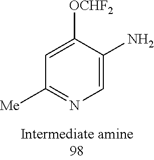 [SMILES: Cc1cc(OC(F)F)c(N)cn1] | 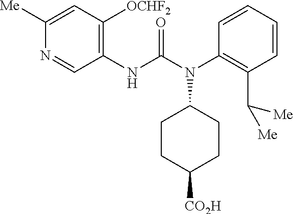 [SMILES: Cc1cc(OC(F)F)c(NC(=O)N(c2ccccc2C(C)C)[C@H]2CC[C@H](C(=O)O)CC2)cn1] | 462.2 [M + H]⁺ |
| | | trans-4-(3-(4-(difluoromethoxy)-6- | |
| | | methylpyridin-3-yl)-1-(2- | |
| | | isopropylphenyl)ureido)cyclohexane-1- | |
| | | carboxylic acid | |
| 158 | 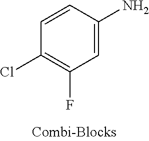 | 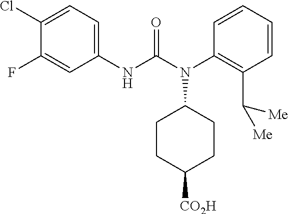 [SMILES: CC(C)c1ccccc1N(C(=O)Nc1ccc(Cl)c(F)c1)[C@H]1CC[C@H](C(=O)O)CC1] | 433.2 [M + H]⁺ |
| | | trans-4-(3-(4-chloro-3-fluorophenyl)-1-(2- | |
| | | isopropylphenyl)ureido)cyclohexane-1- | |
| | | carboxylic acid | |
| 231 | 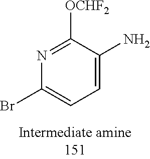 [SMILES: Nc1ccc(Br)nc1OC(F)F] | 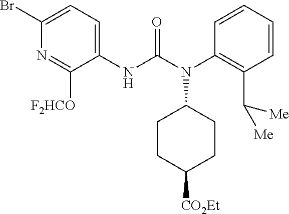 [SMILES: CCOC(=O)[C@H]1CC[C@H](N(C(=O)Nc2ccc(Br)nc2OC(F)F)c2ccccc2C(C)C)CC1] | 554.0, 556.0 [M + H]⁺ |
| | | ethyl (1r,4r)-4-(3-(6-bromo-2- | |
| | | (difluoromethoxy)pyridin-3-yl)-1-(2- | |
| | | isopropylphenyl)ureido)cyclohexane-1- | |
| | | carboxylate | |
| 252 | 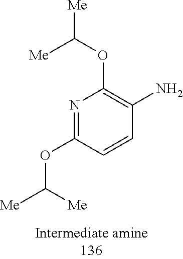 [SMILES: CC(C)Oc1ccc(N)c(OC(C)C)n1] | 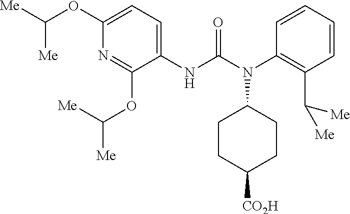 [SMILES: CC(C)Oc1ccc(NC(=O)N(c2ccccc2C(C)C)[C@H]2CC[C@H](C(=O)O)CC2)c(OC(C)C)n1] | 497.9 [M + H]⁺ |
| | | (1r,4r)-4-(3-(2,6-diisopropoxypyridin-3-yl)-1- | |
| | | (2-isopropylphenyl)ureido)cyclohexane-1- | |
| | | carboxylic acid | |
| 253 | 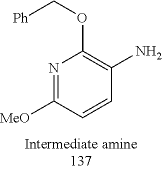 [SMILES: COc1ccc(N)c(OCc2ccccc2)n1] | 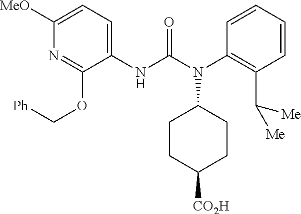 [SMILES: COc1ccc(NC(=O)N(c2ccccc2C(C)C)[C@H]2CC[C@H](C(=O)O)CC2)c(OCc2ccccc2)n1] | 517.9 [M + H]⁺ |
| | | (1r,4r)-4-(3-(2-(benzyloxy)-6-methoxypyridin- | |
| | | 3-yl)-1-(2- | |
| | | isopropylphenyl)ureido)cyclohexane-1- | |
| | | carboxylic acid | |
| 259 | 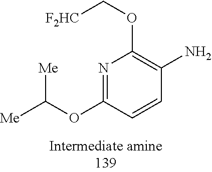 [SMILES: CC(C)Oc1ccc(N)c(OCC(F)F)n1] | 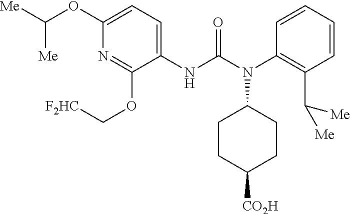 [SMILES: CC(C)Oc1ccc(NC(=O)N(c2ccccc2C(C)C)[C@H]2CC[C@H](C(=O)O)CC2)c(OCC(F)F)n1] | 519.8 [M + H]⁺ |
| | | (1r,4r)-4-(3-(2-(2,2-difluoroethoxy)-6- | |
| | | isopropoxypyridin-3-yl)-1-(2- | |
| | | isopropylphenyl)ureido)cyclohexane-1- | |
| | | carboxylic acid | |
| 288 | 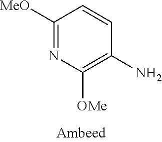 [SMILES: COc1ccc(N)c(OC)n1] | 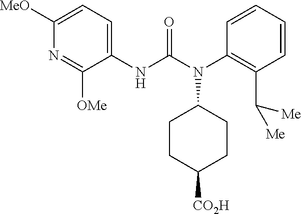 [SMILES: COc1ccc(NC(=O)N(c2ccccc2C(C)C)[C@H]2CC[C@H](C(=O)O)CC2)c(OC)n1] | 442.2 [M + H]⁺ |
| | | (1r,4r)-4-(3-(2,6-dimethoxypyridin-3-yl)-1-(2- | |
| | | isopropylphenyl)ureido)cyclohexane-1- | |
| | | carboxylic acid | |
| 289 | 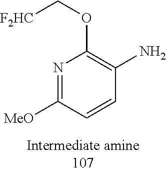 [SMILES: COc1ccc(N)c(OCC(F)F)n1] | 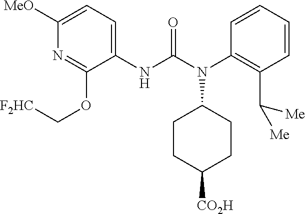 [SMILES: COc1ccc(NC(=O)N(c2ccccc2C(C)C)[C@H]2CC[C@H](C(=O)O)CC2)c(OCC(F)F)n1] | 492.2 [M + H]⁺ |
| | | (1r,4r)-4-(3-(2-(2,2-difluoroethoxy)-6- | |
| | | methoxypyridin-3-yl)-1-(2- | |
| | | isopropylphenyl)ureido)cyclohexane-1- | |
| | | carboxylic acid | |
| 294 | 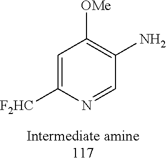 [SMILES: COc1cc(C(F)F)ncc1N] | 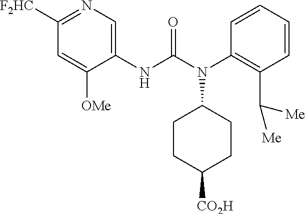 [SMILES: COc1cc(C(F)F)ncc1NC(=O)N(c1ccccc1C(C)C)[C@H]1CC[C@H](C(=O)O)CC1] | 462.2 [M + H]⁺ |
| | | (1r,4r)-4-(3-(6-(difluoromethyl)-4- | |
| | | methoxypyridin-3-yl)-1-(2- | |
| | | isopropylphenyl)ureido)cyclohexane-1- | |
| | | carboxylic acid | |
| 295 | 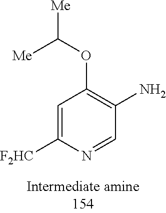 [SMILES: CC(C)Oc1cc(C(F)F)ncc1N] | 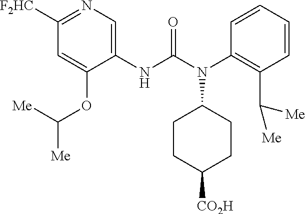 [SMILES: CC(C)Oc1cc(C(F)F)ncc1NC(=O)N(c1ccccc1C(C)C)[C@H]1CC[C@H](C(=O)O)CC1] | 490.3 [M + H]⁺ |
| | | (1r,4r)-4-(3-(6-(difluoromethyl)-4- | |
| | | isopropoxypyridin-3-yl)-1-(2- | |
| | | isopropylphenyl)ureido)cyclohexane-1- | |
| | | carboxylic acid | |
| 296 | 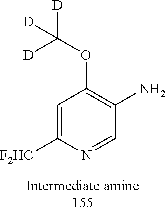 [SMILES: [2H]C([2H])([2H])Oc1cc(C(F)F)ncc1N] | 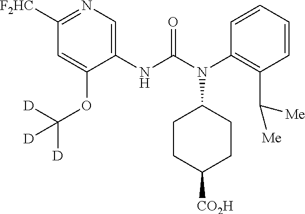 [SMILES: [2H]C([2H])([2H])Oc1cc(C(F)F)ncc1NC(=O)N(c1ccccc1C(C)C)[C@H]1CC[C@H](C(=O)O)CC1] | 465.2 [M + H]⁺ |
| | | (1r,4r)-4-(3-(6-(difluoromethyl)-4-(methoxy- | |
| | | d₃)pyridin-3-yl)-1-(2- | |
| | | isopropylphenyl)ureido)cyclohexane-1- | |
| | | carboxylic acid | |
| 298 | 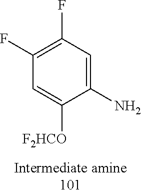 [SMILES: Nc1cc(F)c(F)cc1OC(F)F] | 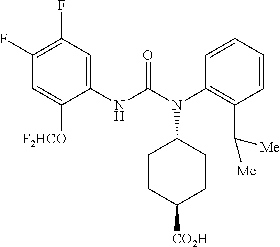 [SMILES: CC(C)c1ccccc1N(C(=O)Nc1cc(F)c(F)cc1OC(F)F)[C@H]1CC[C@H](C(=O)O)CC1] | 483.5 [M + H]⁺ |
| | | (1r,4r)-4-(3-(2-(difluoromethoxy)-4,5- | |
| | | difluorophenyl)-1-(2- | |
| | | isopropylphenyl)ureido)cyclohexane-1- | |
| | | carboxylic acid | |
| 299 | 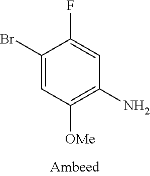 [SMILES: COc1cc(Br)c(F)cc1N] | 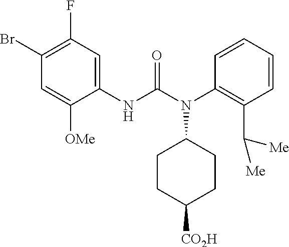 [SMILES: COc1cc(Br)c(F)cc1NC(=O)N(c1ccccc1C(C)C)[C@H]1CC[C@H](C(=O)O)CC1] | 507.2, 509.2 [M + H]⁺ |
| | | (1r,4r)-4-(3-(4-bromo-5-fluoro-2- | |
| | | methoxyphenyl)-1-(2- | |
| | | isopropylphenyl)ureido)cyclohexane-1- | |
| | | carboxylic acid | |
| 300 | 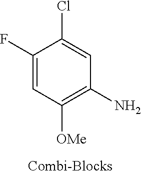 [SMILES: COc1cc(F)c(Cl)cc1N] | 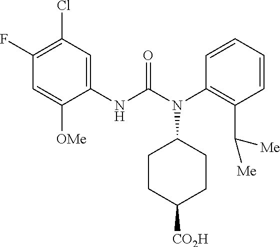 [SMILES: COc1cc(F)c(Cl)cc1NC(=O)N(c1ccccc1C(C)C)[C@H]1CC[C@H](C(=O)O)CC1] | 463.2, 465.2 [M + H]⁺ |
| | | (1r,4r)-4-(3-(5-chloro-4-fluoro-2- | |
| | | methoxyphenyl)-1-(2- | |
| | | isopropylphenyl)ureido)cyclohexane-1- | |
| | | carboxylic acid | |
| 315 | 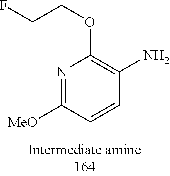 [SMILES: COc1ccc(N)c(OCCF)n1] | 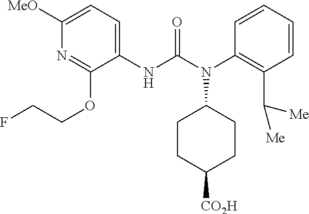 [SMILES: COc1ccc(NC(=O)N(c2ccccc2C(C)C)[C@H]2CC[C@H](C(=O)O)CC2)c(OCCF)n1] | 474.2 [M + H]⁺ |
| | | (1r,4r)-4-(3-(2-(2-fluoroethoxy)-6- | |
| | | methoxypyridin-3-yl)-1-(2- | |
| | | isopropylphenyl)ureido)cyclohexane-1- | |
| | | carboxylic acid | |
| 316 | 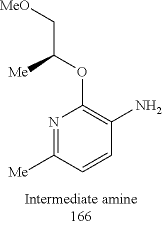 [SMILES: COC[C@H](C)Oc1nc(C)ccc1N] | 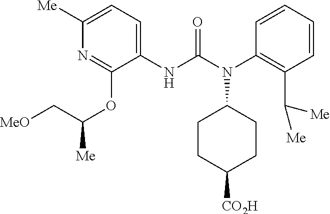 [SMILES: COC[C@H](C)Oc1nc(C)ccc1NC(=O)N(c1ccccc1C(C)C)[C@H]1CC[C@H](C(=O)O)CC1] | 484.3 [M + H]⁺ |
| | | trans-4-(1-(2-isopropylphenyl)-3-(2-(((S)-1- | |
| | | methoxypropan-2-yl)oxy)-6-methylpyridin-3- | |
| | | yl)ureido)cyclohexane-1-carboxylic acid | |
| 317 | 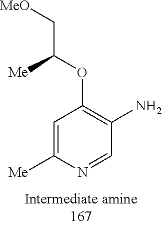 [SMILES: COC[C@H](C)Oc1cc(C)ncc1N] | 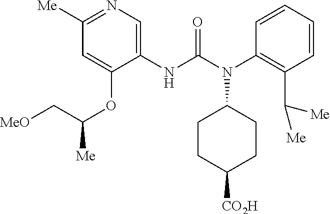 [SMILES: COC[C@H](C)Oc1cc(C)ncc1NC(=O)N(c1ccccc1C(C)C)[C@H]1CC[C@H](C(=O)O)CC1] | 484.3 [M + H]⁺ |
| | | trans-4-(1-(2-isopropylphenyl)-3-(4-(((S)-1- | |
| | | methoxypropan-2-yl)oxy)-6-methylpyridin-3- | |
| | | yl)ureido)cyclohexane-1-carboxylic acid | |
| 318 | 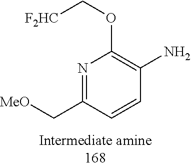 [SMILES: COCc1ccc(N)c(OCC(F)F)n1] | 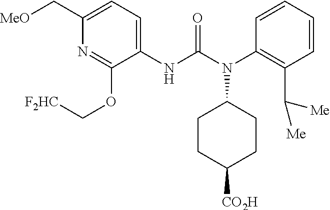 [SMILES: COCc1ccc(NC(=O)N(c2ccccc2C(C)C)[C@H]2CC[C@H](C(=O)O)CC2)c(OCC(F)F)n1] | 506.2 [M + H]⁺ |
| | | (1r,4r)-4-(3-(2-(2,2-difluoroethoxy)-6- | |
| | | (methoxymethyl)pyridin-3-yl)-1-(2- | |
| | | isopropylphenyl)ureido)cyclohexane-1- | |
| | | carboxylic acid | |
| 319 | 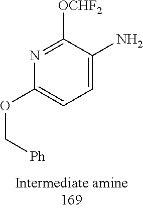 [SMILES: Nc1ccc(OCc2ccccc2)nc1OC(F)F] | 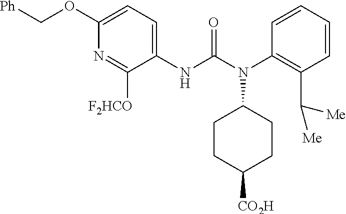 [SMILES: CC(C)c1ccccc1N(C(=O)Nc1ccc(OCc2ccccc2)nc1OC(F)F)[C@H]1CC[C@H](C(=O)O)CC1] | 554.2 [M + H]⁺ |
| | | (1r,4r)-4-(3-(6-(benzyloxy)-2- | |
| | | (difluoromethoxy)pyridin-3-yl)-1-(2- | |
| | | isopropylphenyl)ureido)cyclohexane-1- | |
| | | carboxylic acid | |
| 327 | 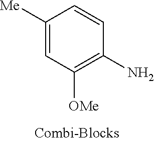 | 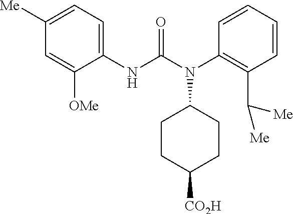 [SMILES: COc1cc(C)ccc1NC(=O)N(c1ccccc1C(C)C)[C@H]1CC[C@H](C(=O)O)CC1] | 425.5 [M + H]⁺ |
| | | (1r,4r)-4-(1-(2-isopropylphenyl)-3-(2- | |
| | | methoxy-4-methylphenyl)ureido)cyclohexane- | |
| | | 1-carboxylic acid | |
| 328 | 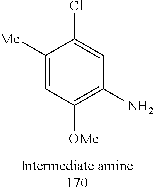 [SMILES: COc1cc(C)c(Cl)cc1N] | 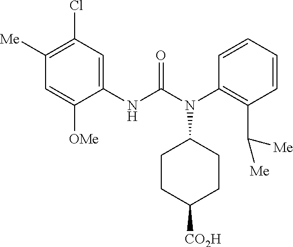 [SMILES: COc1cc(C)c(Cl)cc1NC(=O)N(c1ccccc1C(C)C)[C@H]1CC[C@H](C(=O)O)CC1] | 459.1, 461.1 [M + H]⁺ |
| | | (1r,4r)-4-(3-(5-chloro-2-methoxy-4- | |
| | | methylphenyl)-1-(2- | |
| | | isopropylphenyl)ureido)cyclohexane-1- | |
| | | carboxylic acid | |
| 333 | 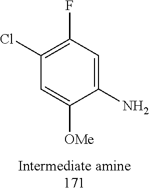 [SMILES: COc1cc(Cl)c(F)cc1N] | 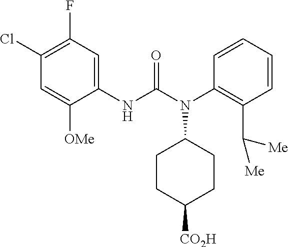 [SMILES: COc1cc(Cl)c(F)cc1NC(=O)N(c1ccccc1C(C)C)[C@H]1CC[C@H](C(=O)O)CC1] | 463.2, 465.2 [M + H]⁺ |
| | | (1r,4r)-4-(3-(4-chloro-5-fluoro-2- | |
| | | methoxyphenyl)-1-(2- | |
| | | isopropylphenyl)ureido)cyclohexane-1- | |
| | | carboxylic acid | |
| 334 | 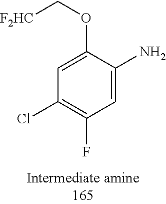 [SMILES: Nc1cc(F)c(Cl)cc1OCC(F)F] | 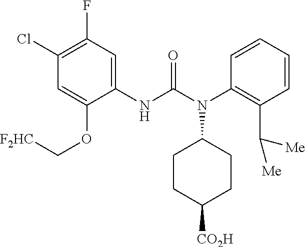 [SMILES: CC(C)c1ccccc1N(C(=O)Nc1cc(F)c(Cl)cc1OCC(F)F)[C@H]1CC[C@H](C(=O)O)CC1] | 513.2, 515.2 [M + H]⁺ |
| | | (1r,4r)-4-(3-(4-chloro-2-(2,2-difluoroethoxy)- | |
| | | 5-fluorophenyl)-1-(2- | |
| | | isopropylphenyl)ureido)cyclohexane-1- | |
| | | carboxylic acid | |
| 335 | 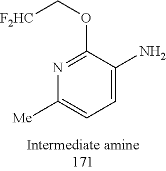 [SMILES: Cc1ccc(N)c(OCC(F)F)n1] | 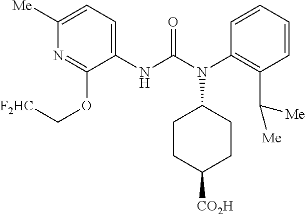 [SMILES: Cc1ccc(NC(=O)N(c2ccccc2C(C)C)[C@H]2CC[C@H](C(=O)O)CC2)c(OCC(F)F)n1] | 476.3 [M + H]⁺ |
| | | (1r,4r)-4-(3-(2-(2,2-difluoroethoxy)-6- | |
| | | methylpyridin-3-yl)-1-(2- | |
| | | isopropylphenyl)ureido)cyclohexane-1- | |
| | | carboxylic acid | |
| 336 | 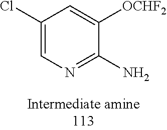 [SMILES: Nc1ncc(Cl)cc1OC(F)F] | 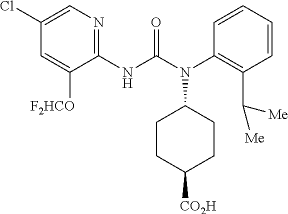 [SMILES: CC(C)c1ccccc1N(C(=O)Nc1ncc(Cl)cc1OC(F)F)[C@H]1CC[C@H](C(=O)O)CC1] | 482.3, 484.3 [M + H]⁺ |
| | | (1r,4r)-4-(3-(5-chloro-3- | |
| | | (difluoromethoxy)pyridin-2-yl)-1-(2- | |
| | | isopropylphenyl)ureido)cyclohexane-1- | |
| | | carboxylic acid | |
| 338 | 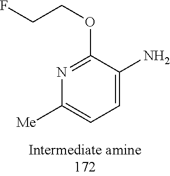 [SMILES: Cc1ccc(N)c(OCCF)n1] | 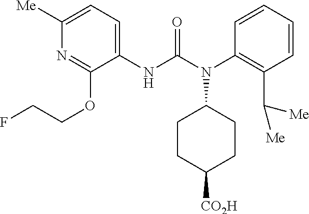 [SMILES: Cc1ccc(NC(=O)N(c2ccccc2C(C)C)[C@H]2CC[C@H](C(=O)O)CC2)c(OCCF)n1] | 458.2 [M + H]⁺ |
| | | (1r,4r)-4-(3-(2-(2-fluoroethoxy)-6- | |
| | | methylpyridin-3-yl)-1-(2- | |
| | | isopropylphenyl)ureido)cyclohexane-1- | |
| | | carboxylic acid | |
| 339 | 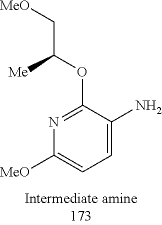 [SMILES: COC[C@H](C)Oc1nc(OC)ccc1N] | 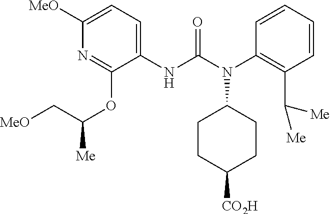 [SMILES: COC[C@H](C)Oc1nc(OC)ccc1NC(=O)N(c1ccccc1C(C)C)[C@H]1CC[C@H](C(=O)O)CC1] | 500.3 [M + H]⁺ |
| | | trans-4-(1-(2-isopropylphenyl)-3-(6-methoxy- | |
| | | 2-(((S)-1-methoxypropan-2-yl)oxy)pyridin-3- | |
| | | yl)ureido)cyclohexane-1-carboxylic acid | |

The following examples were prepared in an analogous fashion to Example 148, but substituting Intermediate amine 95 with the requisite amine. In Example 159, step 2 was obviated.

| | | | LCMS |
|---|---|---|---|
| Example | Starting Amine | Structure | (m/z) |
| 159 | 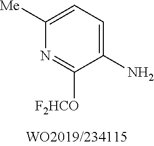 [SMILES: Cc1ccc(N)c(OC(F)F)n1] | 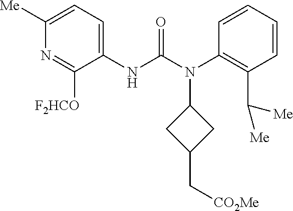 [SMILES: COC(=O)CC1CC(N(C(=O)Nc2ccc(C)nc2OC(F)F)c2ccccc2C(C)C)C1] | 462.1 [M + H]⁺ |
| | | methyl 2-(3-(3-(2-(difluoromethoxy)-6- | |
| | | methylpyridin-3-yl)-1-(2- | |
| | | isopropylphenyl)ureido)cyclobutyl)acetate | |
| 160 | 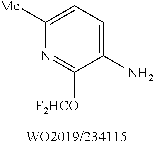 [SMILES: Cc1ccc(N)c(OC(F)F)n1] | 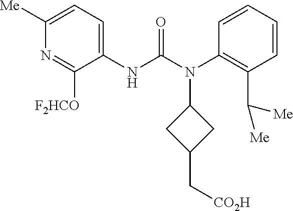 [SMILES: Cc1ccc(NC(=O)N(c2ccccc2C(C)C)C2CC(CC(=O)O)C2)c(OC(F)F)n1] | 448.0 [M + H]⁺ |
| | | 2-(3-(3-(2-(difluoromethoxy)-6-methylpyridin-3- | |
| | | yl)-1-(2- | |
| | | isopropylphenyl)ureido)cyclobutyl)acetic acid | |

The following examples were prepared in an analogous fashion to Example 151 and Example 152, but substituting Intermediate amine 95 with the requisite amine. In Example 163, step 2 was obviated.

| | | | LCMS |
|---|---|---|---|
| Example | Starting Amine | Structure | (m/z) |
| 161 | 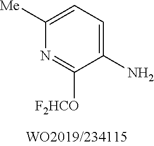 [SMILES: Cc1ccc(N)c(OC(F)F)n1] | 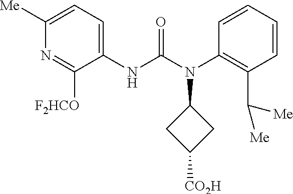 [SMILES: Cc1ccc(NC(=O)N(c2ccccc2C(C)C)[C@H]2C[C@H](C(=O)O)C2)c(OC(F)F)n1] | 434.1 [M + H]⁺ |
| | | trans-3-(3-(2-(difluoromethoxy)-6- | |
| | | methylpyridin-3-yl)-1-(2- | |
| | | isopropylphenyl)ureido)cyclobutane-1- | |
| | | carboxylic acid | |
| 162 | 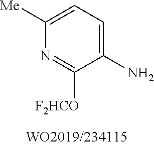 [SMILES: Cc1ccc(N)c(OC(F)F)n1] | 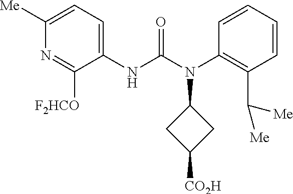 [SMILES: Cc1ccc(NC(=O)N(c2ccccc2C(C)C)[C@H]2C[C@@H](C(=O)O)C2)c(OC(F)F)n1] | 434.1 [M + H]⁺ |
| | | cis-3-(3-(2-(difluoromethoxy)-6-methylpyridin- | |
| | | 3-yl)-1-(2-isopropylphenyl)ureido)cyclobutane- | |
| | | 1-carboxylic acid | |
| 163 | 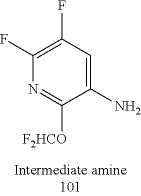 [SMILES: Nc1cc(F)c(F)nc1OC(F)F] | 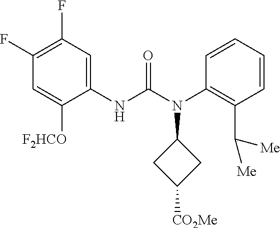 [SMILES: COC(=O)[C@H]1C[C@H](N(C(=O)Nc2cc(F)c(F)cc2OC(F)F)c2ccccc2C(C)C)C1] | 469.0 [M + H]⁺ |
| | | methyl (1r,3r)-3-(3-(2-(difluoromethoxy)-4,5- | |
| | | difluorophenyl)-1-(2- | |
| | | isopropylphenyl)ureido)cyclobutane-1- | |
| | | carboxylate | |
| 164 | 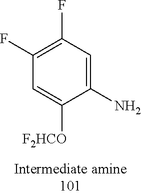 [SMILES: Nc1cc(F)c(F)cc1OC(F)F] | 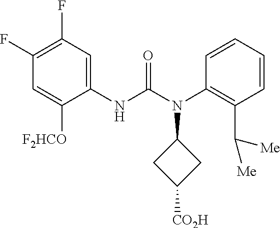 [SMILES: CC(C)c1ccccc1N(C(=O)Nc1cc(F)c(F)cc1OC(F)F)[C@H]1C[C@H](C(=O)O)C1] | 455.0 [M + H]⁺ |
| | | trans-3-(3-(2-(difluoromethoxy)-4,5- | |
| | | difluorophenyl)-1-(2- | |
| | | isopropylphenyl)ureido)cyclobutane-1- | |
| | | carboxylic acid | |

The following examples were prepared in an analogous fashion to Example 153 and Example 154, but substituting Intermediate amine 95 with the requisite amine.

| | | | LCMS |
|---|---|---|---|
| Example | Starting Amine | Structure | (m/z) |
| 165 | 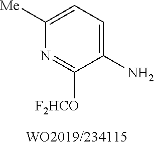 [SMILES: Cc1ccc(N)c(OC(F)F)n1] | 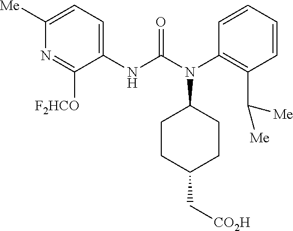 [SMILES: Cc1ccc(NC(=O)N(c2ccccc2C(C)C)[C@H]2CC[C@H](CC(=O)O)CC2)c(OC(F)F)n1] | 476.1 [M + H]⁺ |
| | | 2-((1r,4r)-4-(3-(2-(difluoromethoxy)-6- | |
| | | methylpyridin-3-yl)-1-(2- | |
| | | isopropylphenyl)ureido)cyclohexyl)acetic acid | |
| 166 | 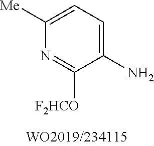 [SMILES: Cc1ccc(N)c(OC(F)F)n1] | 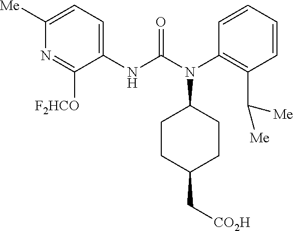 [SMILES: Cc1ccc(NC(=O)N(c2ccccc2C(C)C)[C@H]2CC[C@@H](CC(=O)O)CC2)c(OC(F)F)n1] | 476.1 [M + H]⁺ |
| | | 2-((1s,4s)-4-(3-(2-(difluoromethoxy)-6- | |
| | | methylpyridin-3-yl)-1-(2- | |
| | | isopropylphenyl)ureido)cyclohexyl)acetic acid | |

The following examples were prepared in an analogous fashion to Example 144, but substituting Intermediate amine 95 with the requisite Starting Amine 1, and Intermediate amine 58 with the requisite Starting Amine 2. In Example 169, Example 219 and Example 220, step 2 was obviated. In Example 285, Example 287, Example 305, Example 307, Example 308, Example 309, Example 329, Example 330, Example 331, and Example 332, phosgene (1.5 equiv) in step 1 was also substituted with triphosgene (0.5 equiv, Sigma-Aldrich). Additionally, for Example 308 and Example 309, the reaction was cared out at 50° C. for 5 days in step 2. In Example 301, Example 302, Example 303, Example 304, and Example 337, phosgene (1.5 equiv) was also substituted with triphosgene (0.4 equiv, Sigma-Aldrich), 4-(dimethylamino)pyri dine (1.5 equiv, Sigma-Aldrich) was also included as an additive, and the reaction was also carried at reflux for 16 h in step 1.

| Example | Starting Amine 1 | Starting Amine 2 | Structure | LCMS (m/z) |
|---|---|---|---|---|
| 167 | 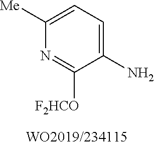 [SMILES: Cc1ccc(N)c(OC(F)F)n1] | 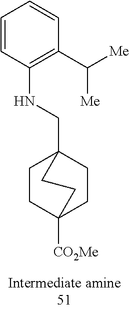 [SMILES: COC(=O)C12CCC(CNc3ccccc3C(C)C)(CC1)CC2] | 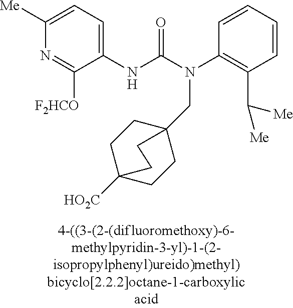 [SMILES: Cc1ccc(NC(=O)N(CC23CCC(C(=O)O)(CC2)CC3)c2ccccc2C(C)C)c(OC(F)F)n1] | 502.1 [M + H]⁺ |
| 168 | 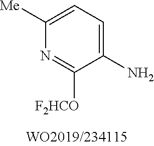 [SMILES: Cc1ccc(N)c(OC(F)F)n1] | 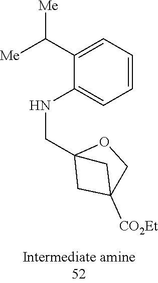 [SMILES: CCOC(=O)C12COC(CNc3ccccc3C(C)C)(C1)C2] | 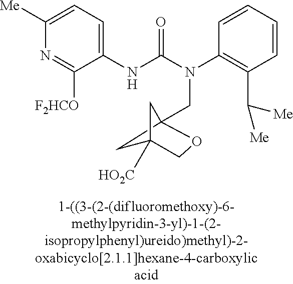 [SMILES: Cc1ccc(NC(=O)N(CC23CC(C(=O)O)(CO2)C3)c2ccccc2C(C)C)c(OC(F)F)n1] | 476.0 [M + H]⁺ |
| 169 | 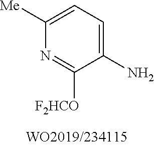 [SMILES: Cc1ccc(N)c(OC(F)F)n1] | 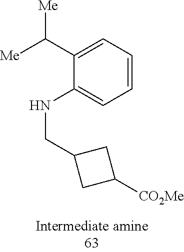 [SMILES: COC(=O)C1CC(CNc2ccccc2C(C)C)C1] | 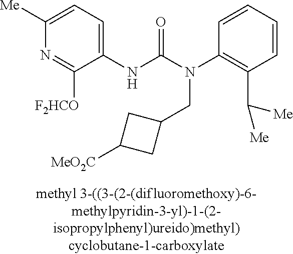 [SMILES: COC(=O)C1CC(CN(C(=O)Nc2ccc(C)nc2OC(F)F)c2ccccc2C(C)C)C1] | 462.1 [M + H]⁺ |
| 170 | 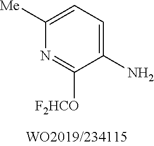 [SMILES: Cc1ccc(N)c(OC(F)F)n1] | 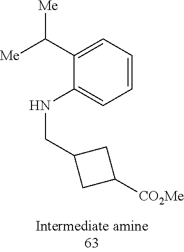 [SMILES: COC(=O)C1CC(CNc2ccccc2C(C)C)C1] | 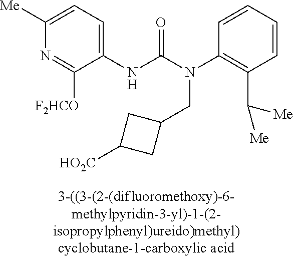 [SMILES: Cc1ccc(NC(=O)N(CC2CC(C(=O)O)C2)c2ccccc2C(C)C)c(OC(F)F)n1] | 448.1 [M + H]⁺ |
| 171 | 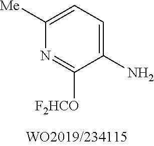 [SMILES: Cc1ccc(N)c(OC(F)F)n1] | 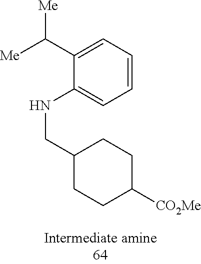 [SMILES: COC(=O)C1CCC(CNc2ccccc2C(C)C)CC1] | 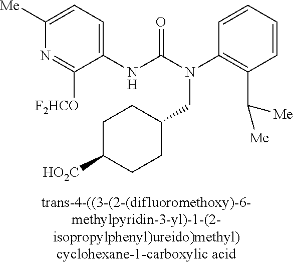 [SMILES: Cc1ccc(NC(=O)N(C[C@H]2CC[C@H](C(=O)O)CC2)c2ccccc2C(C)C)c(OC(F)F)n1] | 476.1 [M + H]⁺ |
| 172 | 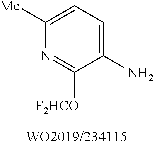 [SMILES: Cc1ccc(N)c(OC(F)F)n1] | 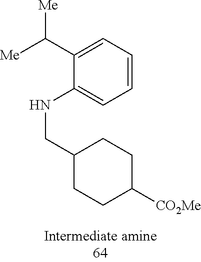 [SMILES: COC(=O)C1CCC(CNc2ccccc2C(C)C)CC1] | 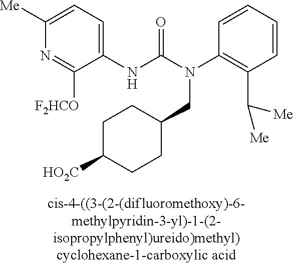 [SMILES: Cc1ccc(NC(=O)N(C[C@H]2CC[C@@H](C(=O)O)CC2)c2ccccc2C(C)C)c(OC(F)F)n1] | 476.1 [M + H]⁺ |
| 173 | 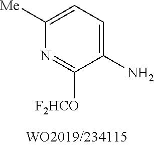 [SMILES: Cc1ccc(N)c(OC(F)F)n1] | 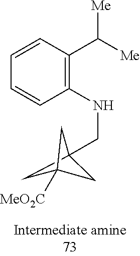 [SMILES: COC(=O)C12CC(CNc3ccccc3C(C)C)(C1)C2] | 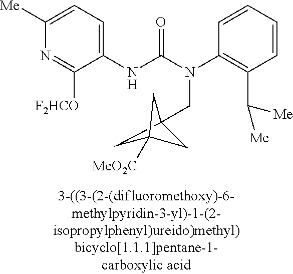 [SMILES: COC(=O)C12CC(CN(C(=O)Nc3ccc(C)nc3OC(F)F)c3ccccc3C(C)C)(C1)C2] | 460.0 [M + H]⁺ |
| 174 | 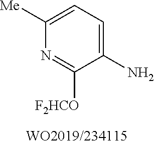 [SMILES: Cc1ccc(N)c(OC(F)F)n1] | 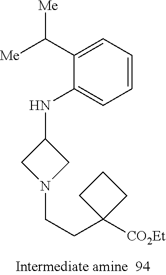 [SMILES: CCOC(=O)C1(CCN2CC(Nc3ccccc3C(C)C)C2)CCC1] | 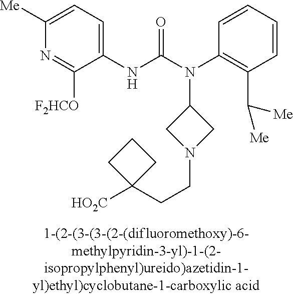 [SMILES: Cc1ccc(NC(=O)N(c2ccccc2C(C)C)C2CN(CCC3(C(=O)O)CCC3)C2)c(OC(F)F)n1] | 517.2 [M + H]⁺ |
| 219 | 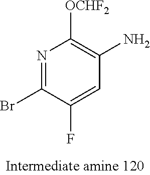 [SMILES: Nc1cc(F)c(Br)nc1OC(F)F] | 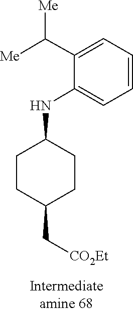 [SMILES: CCOC(=O)C[C@H]1CC[C@@H](Nc2ccccc2C(C)C)CC1] | 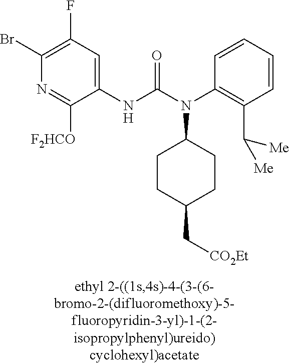 [SMILES: CCOC(=O)C[C@H]1CC[C@@H](N(C(=O)Nc2cc(F)c(Br)nc2OC(F)F)c2ccccc2C(C)C)CC1] | 586.0, 588.0 [M + H]⁺ |
| 220 | 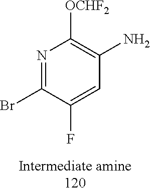 [SMILES: Nc1cc(F)c(Br)nc1OC(F)F] | 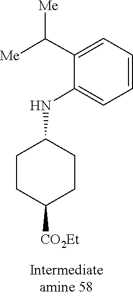 [SMILES: CCOC(=O)[C@H]1CC[C@H](Nc2ccccc2C(C)C)CC1] | 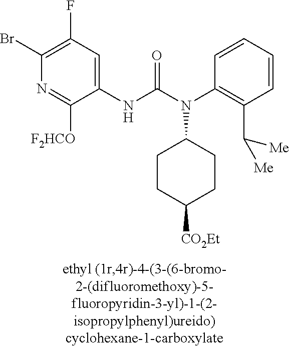 [SMILES: CCOC(=O)[C@H]1CC[C@H](N(C(=O)Nc2cc(F)c(Br)nc2OC(F)F)c2ccccc2C(C)C)CC1] | 572.0, 584.0 [M + H]⁺ |
| 224 | 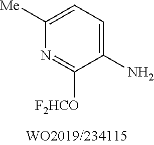 [SMILES: Cc1ccc(N)c(OC(F)F)n1] | 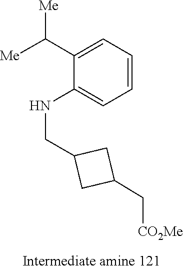 [SMILES: COC(=O)CC1CC(CNc2ccccc2C(C)C)C1] | 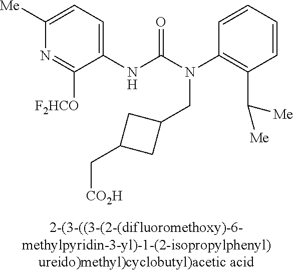 [SMILES: Cc1ccc(NC(=O)N(CC2CC(CC(=O)O)C2)c2ccccc2C(C)C)c(OC(F)F)n1] | 462.1 [M + H]⁺ |
| 227 | 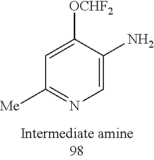 [SMILES: Cc1cc(OC(F)F)c(N)cn1] | 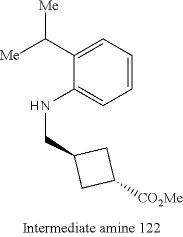 [SMILES: COC(=O)[C@H]1C[C@H](CNc2ccccc2C(C)C)C1] | 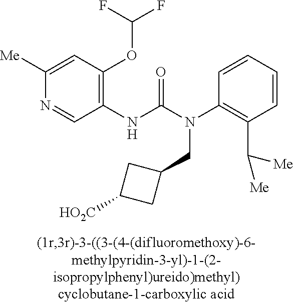 [SMILES: Cc1cc(OC(F)F)c(NC(=O)N(C[C@H]2C[C@H](C(=O)O)C2)c2ccccc2C(C)C)cn1] | 448.2 [M + H]⁺ |
| 228 | 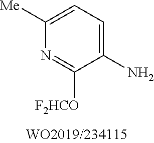 [SMILES: Cc1ccc(N)c(OC(F)F)n1] | 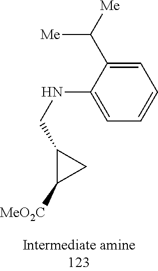 [SMILES: COC(=O)[C@@H]1C[C@H]1CNc1ccccc1C(C)C] | 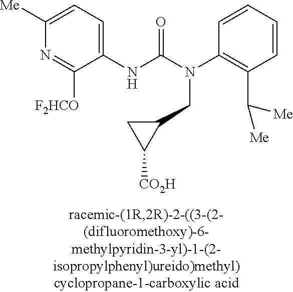 | 434.1 [M + H]⁺ |
| 229 | 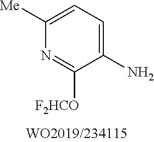 [SMILES: Cc1ccc(N)c(OC(F)F)n1] | 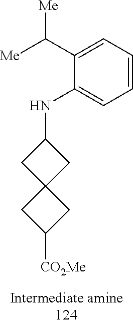 [SMILES: COC(=O)C1CC2(CC(Nc3ccccc3C(C)C)C2)C1] | 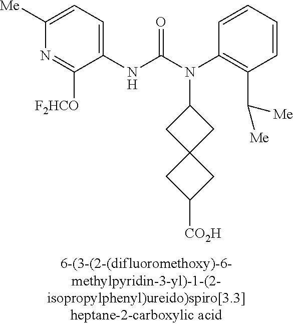 [SMILES: Cc1ccc(NC(=O)N(c2ccccc2C(C)C)C2CC3(CC(C(=O)O)C3)C2)c(OC(F)F)n1] | 474.1 [M + H]⁺ |
| 235 | 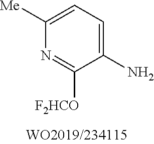 [SMILES: Cc1ccc(N)c(OC(F)F)n1] | 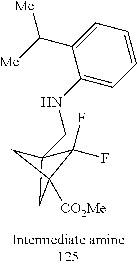 [SMILES: COC(=O)C12CC(CNc3ccccc3C(C)C)(C1)C2(F)F] | 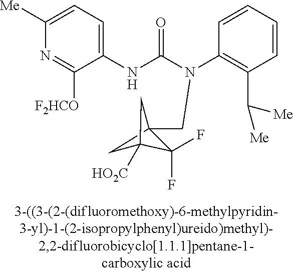 [SMILES: Cc1ccc(NC(=O)N(CC23CC(C(=O)O)(C2)C3(F)F)c2ccccc2C(C)C)c(OC(F)F)n1] | 495.8 [M + H]⁺ |
| 249 | 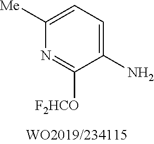 [SMILES: Cc1ccc(N)c(OC(F)F)n1] | 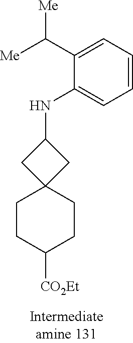 [SMILES: CCOC(=O)C1CCC2(CC1)CC(Nc1ccccc1C(C)C)C2] | 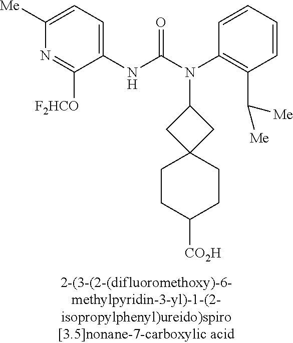 [SMILES: Cc1ccc(NC(=O)N(c2ccccc2C(C)C)C2CC3(CCC(C(=O)O)CC3)C2)c(OC(F)F)n1] | 501.8 [M + H]⁺ |
| 262 | 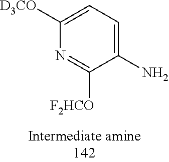 | 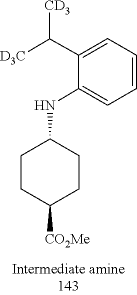 [SMILES: [2H]C([2H])([2H])C(c1ccccc1N[C@H]1CC[C@H](C(=O)OC)CC1)C([2H])([2H])[2H]] | 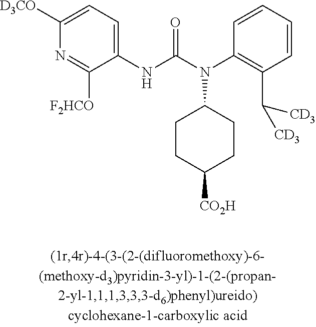 | 486.9 [M + H]⁺ |
| 285 | 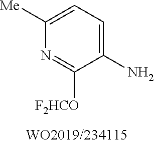 [SMILES: Cc1ccc(N)c(OC(F)F)n1] | 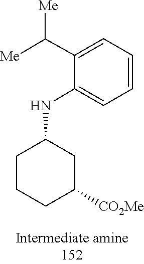 [SMILES: COC(=O)[C@@H]1CCC[C@H](Nc2ccccc2C(C)C)C1] | 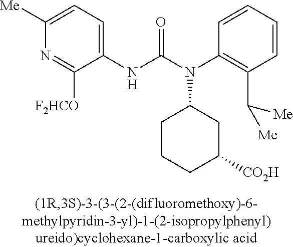 [SMILES: Cc1ccc(NC(=O)N(c2ccccc2C(C)C)[C@H]2CCC[C@@H](C(=O)O)C2)c(OC(F)F)n1] | 462.2 [M + H]⁺ |
| 287 | 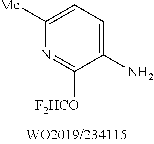 [SMILES: Cc1ccc(N)c(OC(F)F)n1] | 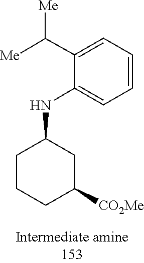 [SMILES: COC(=O)[C@H]1CCC[C@@H](Nc2ccccc2C(C)C)C1] | 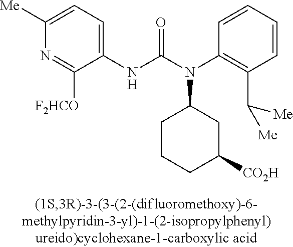 [SMILES: Cc1ccc(NC(=O)N(c2ccccc2C(C)C)[C@@H]2CCC[C@H](C(=O)O)C2)c(OC(F)F)n1] | 462.2 [M + H]⁺ |
| 301 | 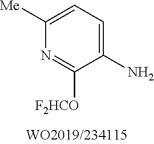 [SMILES: Cc1ccc(N)c(OC(F)F)n1] | 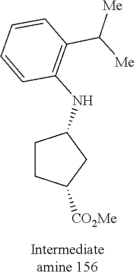 [SMILES: COC(=O)[C@@H]1CC[C@H](Nc2ccccc2C(C)C)C1] | 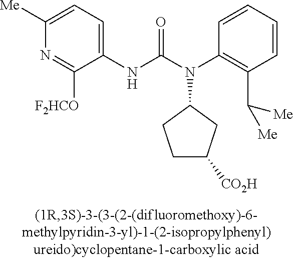 [SMILES: Cc1ccc(NC(=O)N(c2ccccc2C(C)C)[C@H]2CC[C@@H](C(=O)O)C2)c(OC(F)F)n1] | 448.2 [M + H]⁺ |
| 302 | 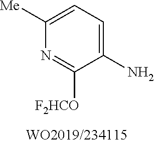 [SMILES: Cc1ccc(N)c(OC(F)F)n1] | 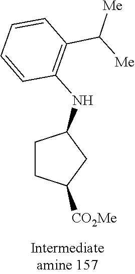 [SMILES: COC(=O)[C@H]1CC[C@@H](Nc2ccccc2C(C)C)C1] | 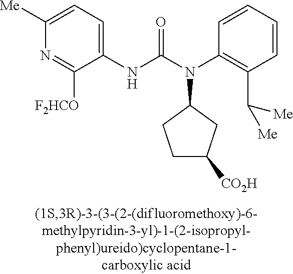 [SMILES: Cc1ccc(NC(=O)N(c2ccccc2C(C)C)[C@@H]2CC[C@H](C(=O)O)C2)c(OC(F)F)n1] | 448.2 [M + H]⁺ |
| 303 | 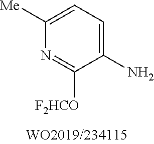 [SMILES: Cc1ccc(N)c(OC(F)F)n1] | 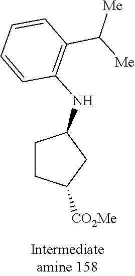 [SMILES: COC(=O)[C@@H]1CC[C@@H](Nc2ccccc2C(C)C)C1] | 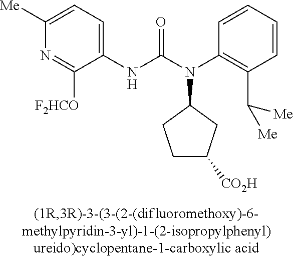 [SMILES: Cc1ccc(NC(=O)N(c2ccccc2C(C)C)[C@@H]2CC[C@@H](C(=O)O)C2)c(OC(F)F)n1] | 448.2 [M + H]⁺ |
| 304 | 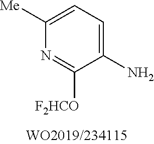 [SMILES: Cc1ccc(N)c(OC(F)F)n1] | 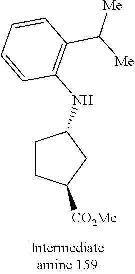 [SMILES: COC(=O)[C@H]1CC[C@H](Nc2ccccc2C(C)C)C1] | 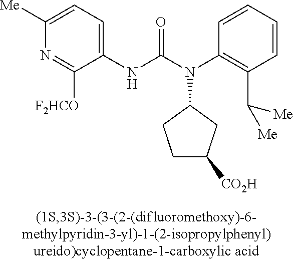 [SMILES: Cc1ccc(NC(=O)N(c2ccccc2C(C)C)[C@H]2CC[C@H](C(=O)O)C2)c(OC(F)F)n1] | 448.2 [M + H]⁺ |
| 305 | 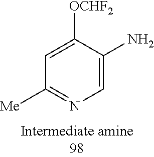 [SMILES: Cc1cc(OC(F)F)c(N)cn1] | 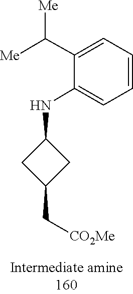 [SMILES: COC(=O)C[C@H]1C[C@@H](Nc2ccccc2C(C)C)C1] | 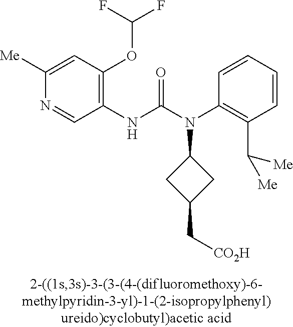 [SMILES: Cc1cc(OC(F)F)c(NC(=O)N(c2ccccc2C(C)C)[C@H]2C[C@@H](CC(=O)O)C2)cn1] | 448.2 [M + H]⁺ |
| 307 | 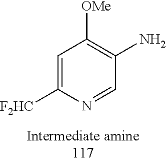 [SMILES: COc1cc(C(F)F)ncc1N] | 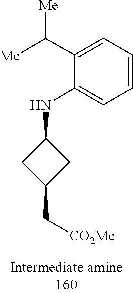 [SMILES: COC(=O)C[C@H]1C[C@@H](Nc2ccccc2C(C)C)C1] | 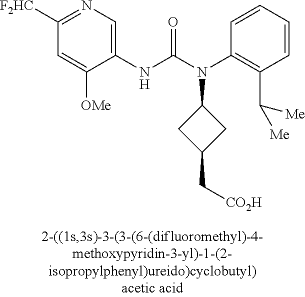 [SMILES: COc1cc(C(F)F)ncc1NC(=O)N(c1ccccc1C(C)C)[C@H]1C[C@@H](CC(=O)O)C1] | 448.3 [M + H]⁺ |
| 308 | 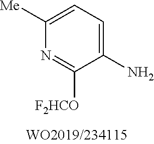 [SMILES: Cc1ccc(N)c(OC(F)F)n1] | 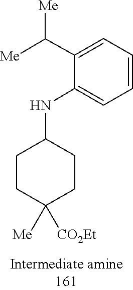 [SMILES: CCOC(=O)C1(C)CCC(Nc2ccccc2C(C)C)CC1] | 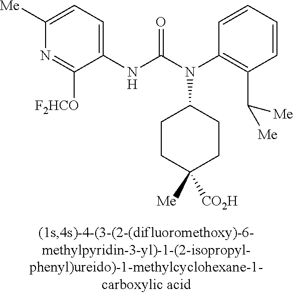 [SMILES: Cc1ccc(NC(=O)N(c2ccccc2C(C)C)[C@H]2CC[C@@](C)(C(=O)O)CC2)c(OC(F)F)n1] | 476.4 [M + H]⁺ |
| 309 | 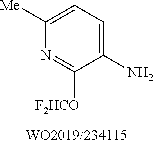 [SMILES: Cc1ccc(N)c(OC(F)F)n1] | 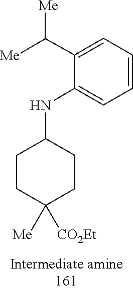 [SMILES: CCOC(=O)C1(C)CCC(Nc2ccccc2C(C)C)CC1] | 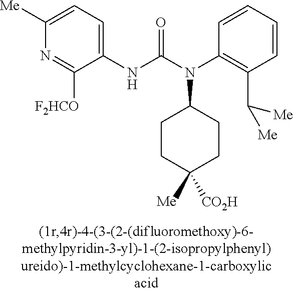 [SMILES: Cc1ccc(NC(=O)N(c2ccccc2C(C)C)[C@H]2CC[C@](C)(C(=O)O)CC2)c(OC(F)F)n1] | 476.4 [M + H]⁺ |
| 329 | 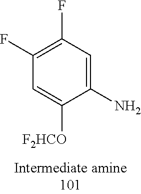 [SMILES: Nc1cc(F)c(F)cc1OC(F)F] | 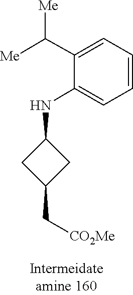 [SMILES: COC(=O)C[C@H]1C[C@@H](Nc2ccccc2C(C)C)C1] | 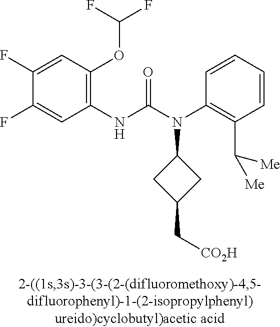 [SMILES: CC(C)c1ccccc1N(C(=O)Nc1cc(F)c(F)cc1OC(F)F)[C@H]1C[C@@H](CC(=O)O)C1] | 469.2 [M + H]⁺ |
| 330 | 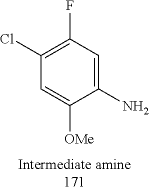 [SMILES: COc1cc(Cl)c(F)cc1N] | 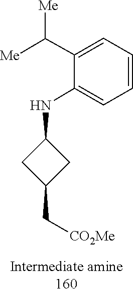 [SMILES: COC(=O)C[C@H]1C[C@@H](Nc2ccccc2C(C)C)C1] | 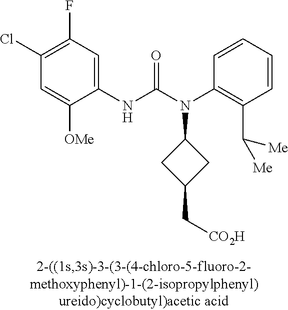 [SMILES: COc1cc(Cl)c(F)cc1NC(=O)N(c1ccccc1C(C)C)[C@H]1C[C@@H](CC(=O)O)C1] | 449.2, 451.2 [M + H]⁺ |
| 331 | 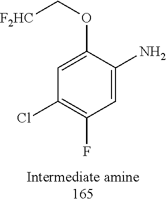 [SMILES: Nc1cc(F)c(Cl)cc1OCC(F)F] | 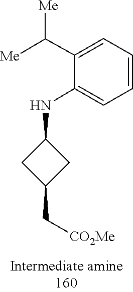 [SMILES: COC(=O)C[C@H]1C[C@@H](Nc2ccccc2C(C)C)C1] | 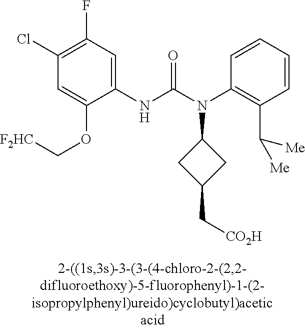 [SMILES: CC(C)c1ccccc1N(C(=O)Nc1cc(F)c(Cl)cc1OCC(F)F)[C@H]1C[C@@H](CC(=O)O)C1] | 499.2, 501.2 [M + H]⁺ |
| 332 | 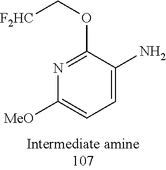 [SMILES: COc1ccc(N)c(OCC(F)F)n1] | 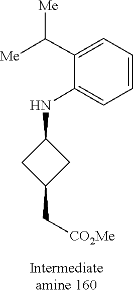 [SMILES: COC(=O)C[C@H]1C[C@@H](Nc2ccccc2C(C)C)C1] | 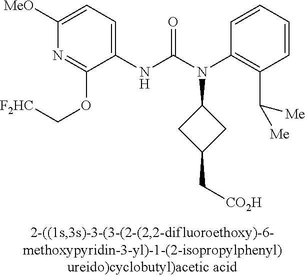 [SMILES: COc1ccc(NC(=O)N(c2ccccc2C(C)C)[C@H]2C[C@@H](CC(=O)O)C2)c(OCC(F)F)n1] | 478.2 [M + H]⁺ |
| 337 | 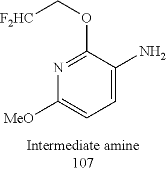 [SMILES: COc1ccc(N)c(OCC(F)F)n1] | 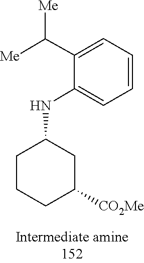 [SMILES: COC(=O)[C@@H]1CCC[C@H](Nc2ccccc2C(C)C)C1] | 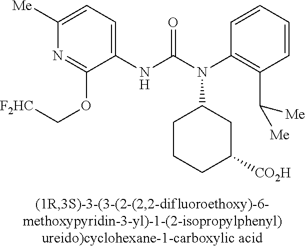 | 492.2 [M + H]⁺ |

### Example 175: Preparation of 2-(3-(3-(5-chloro-3-(difluoromethoxy)pyridin-2-yl)-1-(2-isopropylphenyl)ureido)cyclobutyl)acetic acid

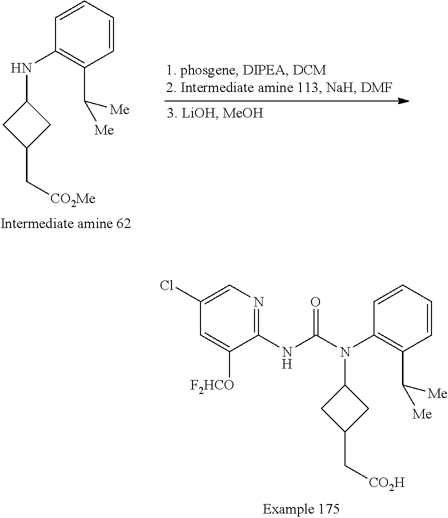 [SMILES: COC(=O)CC1CC(Nc2ccccc2C(C)C)C1 ; CC(C)c1ccccc1N(C(=O)Nc1ncc(Cl)cc1OC(F)F)C1CC(CC(=O)O)C1]

Step 1: In a dried, round-bottom flask equipped with a magnetic stirrer was combined Intermediate amine 62 (1 equiv) and N,N-diisopropylethylamine (3 equiv, Sigma-Aldrich) in anhydrous dichloromethane (0.083 M). To this was then added phosgene (15% w/w solution in toluene, 1.5 equiv, Sigma-Aldrich) dropwise at RT, and the resulting solution was stirred at RT for 10 min. The volatiles were then removed in vacuo and the resulting residue was partitioned between water and dichloromethane. The organic layer was separated, washed further with brine, dried over MgSO₄, and filtered. Concentration of the filtrate thus obtained in vacuo furnished crude methyl 2-(3-((chlorocarbonyl)(2-isopropylphenyl)amino)cyclobutyl)acetate as a tan solid. In a separate dried, round-bottom flask equipped with a magnetic stirrer was dissolved Intermediate amine 113 (1.1 equiv) in DMF (0.17 M). To this was then added sodium hydride (60% w/w dispersion in paraffin oil, 1.1 equiv, Sigma-Aldrich) in one rapid portion and the resulting mixture was stirred at RT for 15 min to afford a yellow-orange solution. Finally, this was added dropwise at RT to a DMF solution (0.17 M) of crude methyl 2-(3-((chlorocarbonyl)(2-isopropylphenyl)amino)cyclobutyl)acetate prepared above. The reaction mixture thus obtained was stirred at RT for another 10 min before it was carefully quenched with saturated aq. NH₄Cl and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded methyl 2-(3-(3-(5-chloro-3-(difluoromethoxy)pyridin-2-yl)-1-(2-isopropylphenyl)ureido)cyclobutyl)acetate as a white foam (58% yield).

Step 2: In a round-bottom flask equipped with a magnetic stirrer was dissolved methyl 2-(3-(3-(5-chloro-3-(difluoromethoxy)pyridin-2-yl)-1-(2-isopropylphenyl)ureido)cyclobutyl)acetate (1 equiv) from the previous step in methanol (0.1 M). To this colorless solution was then added lithium hydroxide (1 M solution in water, 7 equiv) and the resulting mixture was heated at 45° C. for 1 h. Upon cooling to RT, the reaction mixture was carefully quenched with HCl (1 M solution in water, 7 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded the title compound as a white foam (96% yield). LCMS: m/z=468.0, 470.0 [M+H]⁺; ¹H NMR (DMSO-d₆): δ=11.98 (s, 1H), 8.27˜8.26 (m, 1H), 7.75 (br s, 1H), 7.49˜7.28 (m, 4H), 7.18 (t, J=7.1 Hz, 1H), 7.10 (t, J=74.0 Hz, 0.5H, diastereomer A), 7.09 (t, J=74.0 Hz, 0.5H, diastereomer B), 4.92˜4.80 (m, 0.5H, diastereomer A), 4.62˜4.50 (m, 0.5H, diastereomer B), 3.07˜2.96 (m, 1H), 2.45˜2.06 (m, 5H), 2.00˜1.45 (m, 3H), 1.35˜1.10 (m, 5H).

The following example was prepared in an analogous fashion to Example 175, but substituting Intermediate amine 113 with the requisite amine.

| | | | LCMS |
|---|---|---|---|
| Example | Starting Amine | Structure | (m/z) |
| 176 | 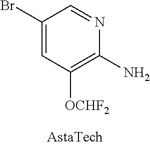 [SMILES: Nc1ncc(Br)cc1OC(F)F] | 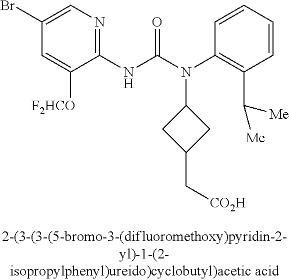 [SMILES: CC(C)c1ccccc1N(C(=O)Nc1ncc(Br)cc1OC(F)F)C1CC(CC(=O)O)C1] | 512.0, 514.0 [M + H]⁺ |

The following examples were prepared in an analogous fashion to Example 176, but substituting Intermediate amine 62 with the requisite amine. In the case of Example 177, step 2 was obviated.

| | | | LCMS |
|---|---|---|---|
| Example | Starting Amine | Structure | (m/z) |
| 177 | 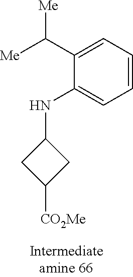 [SMILES: COC(=O)C1CC(Nc2ccccc2C(C)C)C1] | 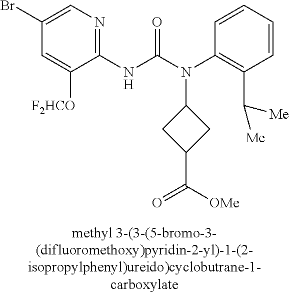 [SMILES: COC(=O)C1CC(N(C(=O)Nc2ncc(Br)cc2OC(F)F)c2ccccc2C(C)C)C1] | 511.9, 513.9 [M + H]⁺ |
| 178 | 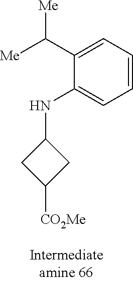 [SMILES: COC(=O)C1CC(Nc2ccccc2C(C)C)C1] | 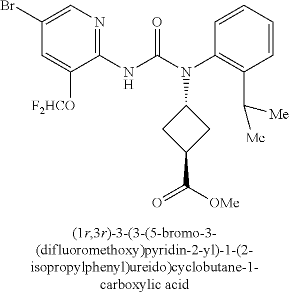 [SMILES: COC(=O)[C@H]1C[C@H](N(C(=O)Nc2ncc(Br)cc2OC(F)F)c2ccccc2C(C)C)C1] | 498.0, 500.0 [M + H]⁺ |
| 179 | 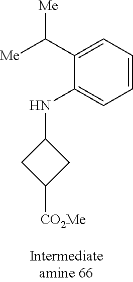 [SMILES: COC(=O)C1CC(Nc2ccccc2C(C)C)C1] | 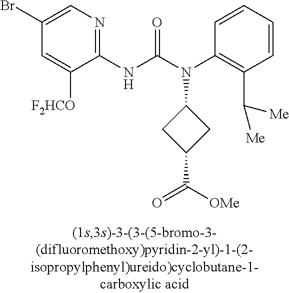 [SMILES: COC(=O)[C@H]1C[C@@H](N(C(=O)Nc2ncc(Br)cc2OC(F)F)c2ccccc2C(C)C)C1] | 498.0, 500.0 [M + H]⁺ |
| 273 | 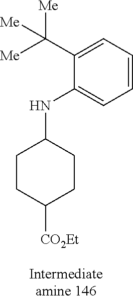 [SMILES: CCOC(=O)C1CCC(Nc2ccccc2C(C)(C)C)CC1] | 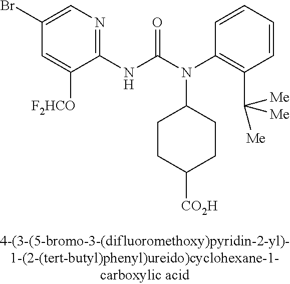 [SMILES: CC(C)(C)c1ccccc1N(C(=O)Nc1ncc(Br)cc1OC(F)F)C1CCC(C(=O)O)CC1] | 540.0, 542.0 [M + H]⁺ |

### Example 180: Preparation of (S)-1-(1-acetylpiperidin-4-yl)-1-(2-isopropylphenyl)-3-(4-((1-methoxypropan-2-yl)oxy)pyridin-3-yl)urea

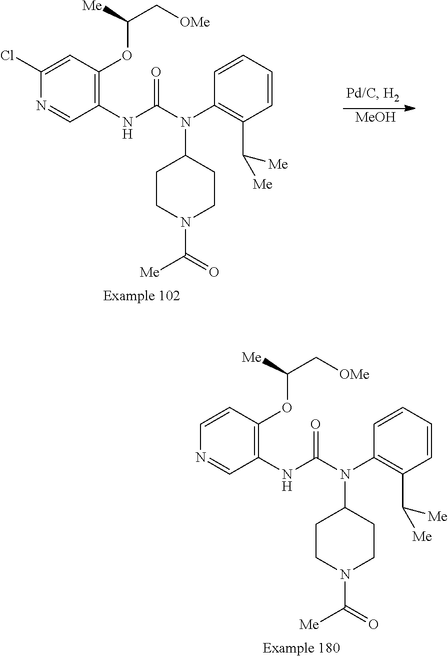 [SMILES: "MeOH" ; COC[C@H](C)Oc1cc(Cl)ncc1NC(=O)N(c1ccccc1C(C)C)C1CCN(C(C)=O)CC1 ; COC[C@H](C)Oc1ccncc1NC(=O)N(c1ccccc1C(C)C)C1CCN(C(C)=O)CC1]

In a dried, round-bottom flask equipped with a magnetic stirrer was dissolved Example 102 (1 equiv) in methanol (0.038 M). The resulting colorless solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere (maintained with a balloon) at RT for 16 h. The reaction was subsequently quenched with dichloromethane and filtered through a bed of dichloromethane-wetted celite. The insolubles were washed further with dichloromethane and the filtrate was concentrated in vacuo. Purification of the crude product thus obtained by way of column chromatography (SiO₂, gradient elution: Hex→1:1 (v/v) Hex: EtOAc) afforded the title compound as a white foam (6400 yield). LCMS: m(z=469.0 [M+H]⁺; ¹H NMR (DMSO-d₆): δ=9.25 (d, J=11.1 Hz, 1H), 8.02 (d, J=5.7 Hz, 1H), 7.63˜7.52 (m, 2H), 7.41˜7.22 (m, 2H), 6.99 (d, J=5.7 Hz, 1H), 6.47 (s, 1H), 4.61˜4.45 (m, 3H), 3.85˜3.75 (m, 1H), 3.18˜3.08 (m, 6H), 2.98˜2.86 (m, 1H), 2.61˜2.53 (m, 2H), 2.10˜1.95 (m, 1H), 1.95 (s, 1.51H, rotamer A), 1.92 (s, 1.51H, rotamer B), 1.83˜1.75 (m, 1H), 1.24˜1.04 (m, 7H), 0.91˜0.87 (in, 3H).

The following examples were prepared in an analogous fashion to Example 180, but substituting Example 102 with the requisite starting material. In the case of Example 183, instead of stirring at RT for 16 h, the reaction was stopped after 20 min.

| | | | LCMS |
|---|---|---|---|
| Example | Starting Material | Structure | (m/z) |
| 181 | 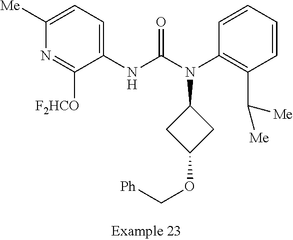 [SMILES: Cc1ccc(NC(=O)N(c2ccccc2C(C)C)[C@H]2C[C@H](OCc3ccccc3)C2)c(OC(F)F)n1] | 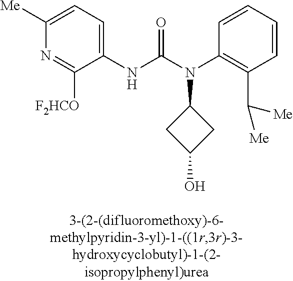 [SMILES: Cc1ccc(NC(=O)N(c2ccccc2C(C)C)[C@H]2C[C@H](O)C2)c(OC(F)F)n1] | 406.1 [M + H]⁺ |
| 182 | 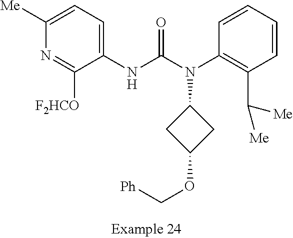 [SMILES: Cc1ccc(NC(=O)N(c2ccccc2C(C)C)[C@H]2C[C@@H](OCc3ccccc3)C2)c(OC(F)F)n1] | 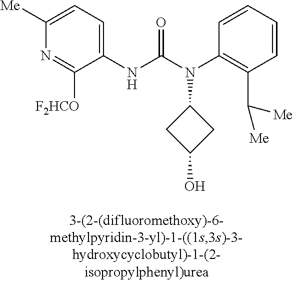 [SMILES: Cc1ccc(NC(=O)N(c2ccccc2C(C)C)[C@H]2C[C@@H](O)C2)c(OC(F)F)n1] | 406.1 [M + H]⁺ |
| 183 | 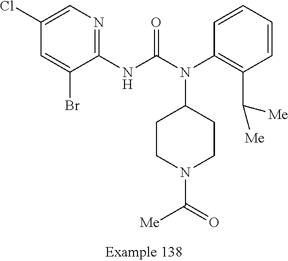 [SMILES: CC(=O)N1CCC(N(C(=O)Nc2ncc(Cl)cc2Br)c2ccccc2C(C)C)CC1] | 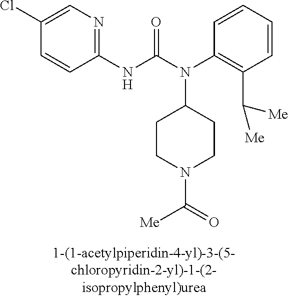 [SMILES: CC(=O)N1CCC(N(C(=O)Nc2ccc(Cl)cn2)c2ccccc2C(C)C)CC1] | 415.1, 417.1 [M + H]⁺ |
| 254 | 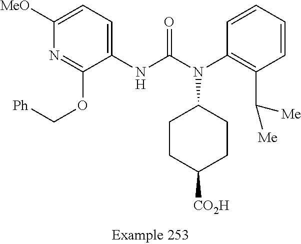 [SMILES: COc1ccc(NC(=O)N(c2ccccc2C(C)C)[C@H]2CC[C@H](C(=O)O)CC2)c(OCc2ccccc2)n1] | 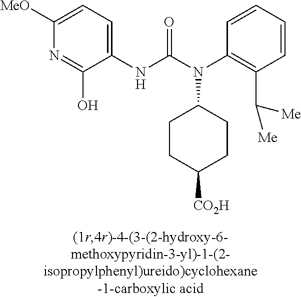 [SMILES: COc1ccc(NC(=O)N(c2ccccc2C(C)C)[C@H]2CC[C@H](C(=O)O)CC2)c(O)n1] | 427.8 [M + H]⁺ |
| 320 | 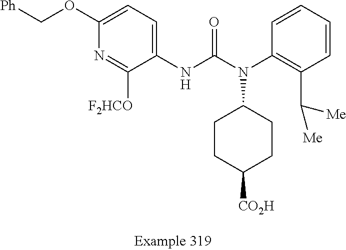 [SMILES: CC(C)c1ccccc1N(C(=O)Nc1ccc(OCc2ccccc2)nc1OC(F)F)[C@H]1CC[C@H](C(=O)O)CC1] | 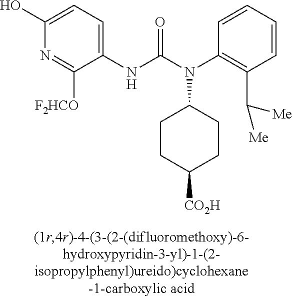 [SMILES: CC(C)c1ccccc1N(C(=O)Nc1ccc(O)nc1OC(F)F)[C@H]1CC[C@H](C(=O)O)CC1] | 464.5 [M + H]⁺ |

### Example 184: Preparation of 1-(1-acetylpiperidin-4-yl)-3-(5-bromo-3-(difluoromethoxy)pyrazin-2-yl)-1-(2-isopropylphenyl)urea

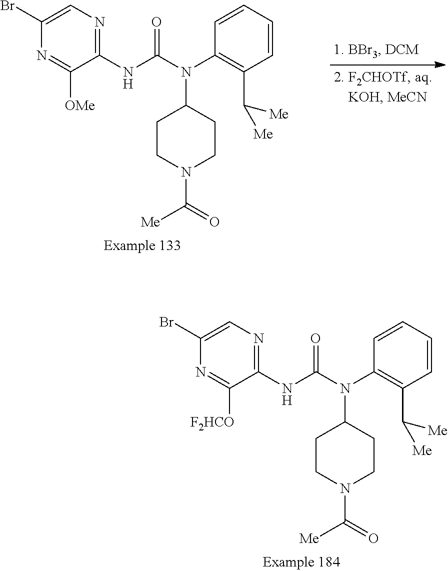 [SMILES: COc1nc(Br)cnc1NC(=O)N(c1ccccc1C(C)C)C1CCN(C(C)=O)CC1 ; CC(=O)N1CCC(N(C(=O)Nc2ncc(Br)nc2OC(F)F)c2ccccc2C(C)C)CC1]

Step 1: In a dried, round-bottom flask equipped with a magnetic stirrer was dissolved Example 133 (1 equiv) in dichloromethane (0.05 M). To the resulting colorless solution was then added boron tribromide (1 M solution in DCM, 1 equiv, Sigma-Aldrich), dropwise over a period of 5 min, and the reaction vessel was heated at 40° C. for 16 h. After cooling to RT, the reaction mixture was diluted further with dichloromethane and carefully quenched with the dropwise addition of saturated aq. NaHCO₃. The aqueous layer was separated and back extracted with dichloromethane. The combined organic extracts were washed further with brine, dried over MgSO₄, filtered, and the filtrate concentrated in vacuo. The crude product thus obtained was subjected to reverse-phase column chromatography (C₁₈, gradient elution: 9:1 (v/v) H₂O: MeCN+0.1% formic acid→MeCN+0.1% formic acid) to afford 1-(1-acetylpiperidin-4-yl)-3-(5-bromo-3-hydroxypyrazin-2-yl)-1-(2-isopropylphenyl)urea as a white foam (38% yield).

Step 2: In a dried, round-bottom flask equipped with a magnetic stirrer was combined 1-(1-acetylpiperidin-4-yl)-3-(5-bromo-3-hydroxypyrazin-2-yl)-1-(2-isopropylphenyl)urea (1 equiv) from the previous step and potassium hydroxide (12 equiv, Alfa Aesar) in a 1:1 (v/v) solution of water and acetonitrile (0.18 M). The resulting solution was cooled to 0° C. and difluoromethyl triflate (3 equiv, Sigma-Aldrich) was then added dropwise over 1 min. The reaction mixture was first stirred at 0° C. for 15 min and then at RT for 10 min, during which time a slight exotherm was observed. The reaction was carefully quenched with the addition of water and then extracted with EtOAc. The combined organic extracts were washed further with brine, dried over MgSO₄, filtered, and the filtrate concentrated in vacuo. The crude product thus obtained was subjected to reverse-phase column chromatography (C₁₈, gradient elution: 9:1 (v/v) H₂O: MeCN+0.1% formic acid→MeCN+0.1% formic acid) to afford the title compound as a white foam (16% yield). LCMS: m/z=526.0, 528.0 [M+H]⁺; ¹H NMR (DMSO-d₆): δ=8.43 (s, 1H), 7.93 (br s, 1H), 7.56 (t, J=71.1 Hz, 1H), 7.48 (dd, J=7.8, 1.4 Hz, 1H), 7.41 (t, J=6.9 Hz, 1H), 7.28 (t, J=7.8 Hz, 1H), 7.18 (d, J=7.8 Hz, 1H), 4.47˜4.32 (m, 2H), 3.88˜3.78 (m, 1H), 3.20˜3.03 (m, 2H), 2.60˜2.54 (m, 1H), 2.06˜1.88 (m, 4H), 1.77˜1.66 (m, 1H), 1.62˜1.06 (m, 8H).

### Example 185: Preparation of 1-(1-acetylpiperidin-4-yl)-3-(6-cyano-2-methoxypyridin-3-yl)-1-(2-isopropylphenyl)urea

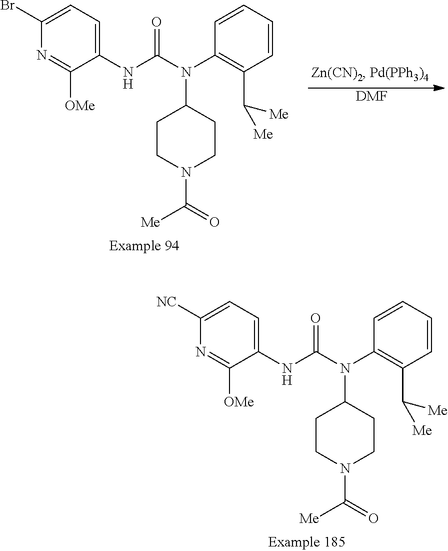

In a thick-walled glass reaction vessel equipped with a magnetic stirrer and a Teflon screwcap was combined Example 94 (1 equiv) and zinc cyanide (5 equiv, Alfa Aesar) in DMF (0.04 M). The resulting mixture was then deoxygenated via sub-surface purging with N₂ for 10 min. Finally, tetrakis(triphenylphosphine)palladium(0) (0.1 equiv, Sigma-Aldrich) was added in one rapid portion, and the reaction vessel was tightly sealed and heated at 110° C. for 3 h. The reaction mixture was then cooled to RT, quenched with water, and extracted with dichloromethane. The combined organic extracts were washed further with brine, dried over Na₂SO₄, filtered, and the filtrate concentrated in vacuo. The crude product thus obtained was subjected to reverse-phase column chromatography (C₁s, gradient elution: 9:1 (v/v) H₂O: MeCN+0.1% formic acid→MeCN+0.1% formic acid) to afford the title compound as a white solid (73% yield). LCMS: m/z=435.9 [M+H]⁺; ¹H NMR (DMSO-d₆): δ=8.49 (d, J=8.1 Hz, 1H), 7.65˜7.53 (m, 3H), 7.40 (t, J=6.9 Hz, 1H), 7.29 (dd, J=7.8, 0.9 Hz, 1H), 6.69 (s, 1H), 4.49˜4.45 (m, 2H), 3.89˜3.78 (m, 1H), 3.65 (s, 3H), 3.16˜2.95 (m, 2H), 2.74˜2.50 (m, 2H), 2.06˜2.00 (m, 1H), 1.95 (s, 1.5H, rotamer A), 1.92 (s, 1.5H, rotamer B), 1.79˜1.75 (m, 1H), 1.59˜1.33 (m, 1H), 1.24˜1.04 (m, 6H).

### Example 186: Preparation of (1r,3r)-3-(3-(2-(difluoromethoxy)-6-methylpyridin-3-yl)-1-(2-isopropylphenyl)ureido)cyclobutane-1-carboxamide

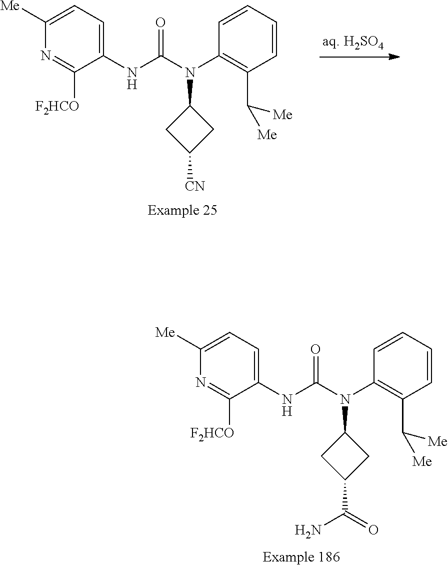 [SMILES: Cc1ccc(NC(=O)N(c2ccccc2C(C)C)[C@H]2C[C@H](C#N)C2)c(OC(F)F)n1 ; Cc1ccc(NC(=O)N(c2ccccc2C(C)C)[C@H]2C[C@H](C(N)=O)C2)c(OC(F)F)n1]

In a glass reaction vessel equipped with a magnetic stirrer was combined Example 25 (1 equiv) with wet sulfuric acid (530 equiv, Fisher Scientific). The resulting mixture was then stirred at RT for 2 h before it was carefully quenched with ice and solid sodium carbonate. The resulting residue was then extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: 4:1 (v/v) Hex: EtOAc→EtOAc) afforded the title compound as a white solid (4400 yield). LCMS: m/z=433.1 [M+H]⁺; ¹H NMR (DMSO-d₆): δ=8.28 (d, J=8.1 Hz, 1H), 7.60˜7.49 (m, 2H), 7.46 (t, J=72.6 Hz, 1H), 7.39 (td, J=7.7, 1.7 Hz, 1H), 7.22 (dd, J=7.7, 1.2 Hz, 1H), 7.16 (s, 1H), 7.05 (d, J=8.2 Hz, 1H), 6.67 (s, 1H), 6.37 (s, 1H), 4.78˜4.66 (m, 1H), 2.96 (sept, J=7.0 Hz, 1H), 2.66˜2.57 (m, 2H), 2.31 (s, 3H), 2.28˜2.18 (m, 2H), 1.89 (q, J=9.6 Hz, 1H), 1.22 (d, J=7.0 Hz, 3H), 1.10 (d, J=7.0 Hz, 3H).

The following examples were prepared in an analogous fashion to Example 186, but substituting Example 25 with the requisite starting nitrile.

| | | | LCMS |
|---|---|---|---|
| Example | Starting Nitrile | Structure | (m/z) |
| 187 | 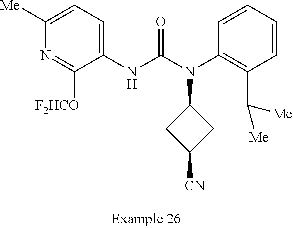 [SMILES: Cc1ccc(NC(=O)N(c2ccccc2C(C)C)[C@H]2C[C@@H](C#N)C2)c(OC(F)F)n1] | 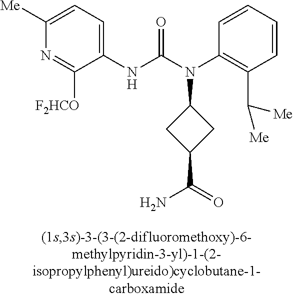 [SMILES: Cc1ccc(NC(=O)N(c2ccccc2C(C)C)[C@H]2C[C@@H](C(N)=O)C2)c(OC(F)F)n1] | 433.1 [M + H]⁺ |
| 188 | 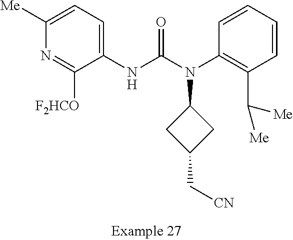 [SMILES: Cc1ccc(NC(=O)N(c2ccccc2C(C)C)[C@H]2C[C@H](CC#N)C2)c(OC(F)F)n1] | 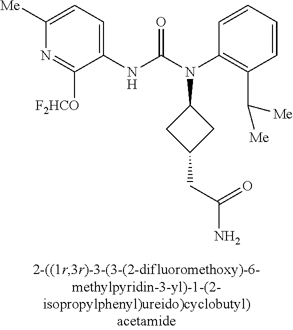 [SMILES: Cc1ccc(NC(=O)N(c2ccccc2C(C)C)[C@H]2C[C@H](CC(N)=O)C2)c(OC(F)F)n1] | 447.1 [M + H]⁺ |
| 189 | 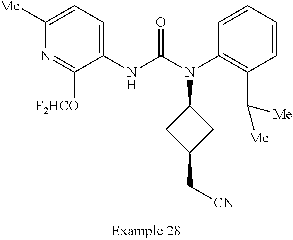 [SMILES: Cc1ccc(NC(=O)N(c2ccccc2C(C)C)[C@H]2C[C@@H](CC#N)C2)c(OC(F)F)n1] | 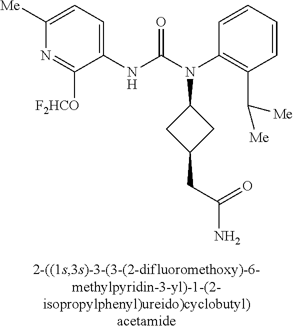 [SMILES: Cc1ccc(NC(=O)N(c2ccccc2C(C)C)[C@H]2C[C@@H](CC(N)=O)C2)c(OC(F)F)n1] | 447.1 [M + H]⁺ |

### Example 190: Preparation of 2-(3-(3-(2-(difluoromethoxy)-6-methylpyridin-3-yl)-1-(2-isopropylphenyl)ureido)cyclobutyl)-N-methylacetamide

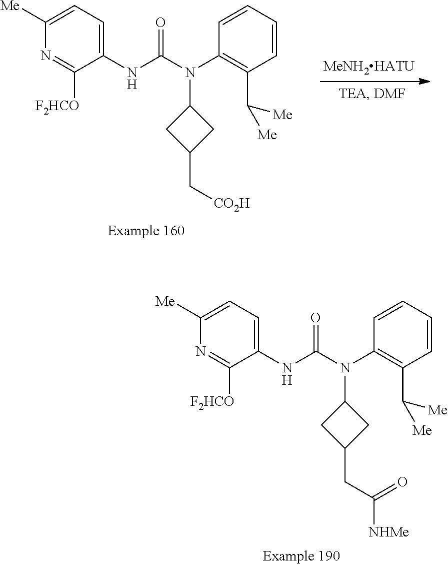 [SMILES: Cc1ccc(NC(=O)N(c2ccccc2C(C)C)C2CC(CC(=O)O)C2)c(OC(F)F)n1 ; CNC(=O)CC1CC(N(C(=O)Nc2ccc(C)nc2OC(F)F)c2ccccc2C(C)C)C1]

In a glass reaction vessel equipped with a magnetic stirrer was combined Example 160 (1 equiv), methylamine (1.2 equiv, 4 M solution in methanol, Sigma-Aldrich), and triethylamine (3.3 equiv, Sigma-Aldrich) in DMF (0.10 M). To this solution was then added 1-[bis(dimethylamino)methylene]-1H-1,2,3-triazolo[4,5-b]pyridinium 3-oxide hexafluorophosphate (1.3 equiv, Chem-Impex) in one rapid portion and the resulting mixture was stirred at RT for 10 min. The reaction was then diluted with water and extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: 10:1 (v/v) Hex: EtOAc→EtOAc) afforded the title compound as a white solid (72% yield). LCMS: m/z=461.1 [M+H]⁺; ¹H NMR (DMSO-d₆): δ=8.30˜8.25 (m, 1H), 7.75˜7.21 (m, 6H), 7.05 (d, J=8.2 Hz, 1H), 6.37 (s, 0.55H, diastereomer A), 6.36 (s, 0.45H, diastereomer B), 5.00˜4.87 (s, 0.55H, diastereomer A), 4.72˜4.59 (m, 0.45H, diastereomer B), 3.00˜2.89 (m, 1H), 2.54 (d, J=4.5 Hz, 1.65H, diastereomer A), 2.48 (d, J=4.5 Hz, 1.35H, diastereomer B), 2.35˜2.07 (m, 7H), 2.00˜1.80 (m, 2H), 1.58˜1.18 (m, 4H), 1.10 (d, J=6.9 Hz, 3H).

### Example 191: Preparation of 1-((1r,3r)-3-((1H-tetrazol-5-yl)methyl)cyclobutyl)-3-(2-(difluoromethoxy)-6-methylpyridin-3-yl)-1-(2-isopropylphenyl)urea

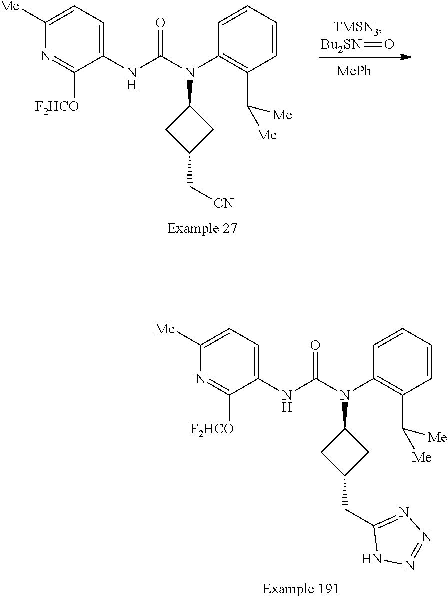

In a glass reaction vessel equipped with a magnetic stirrer was combined Example 27 (1 equiv), trimethylsilyl azide (2 equiv, Sigma-Aldrich), and dibutyltin(IV) oxide (0.1 equiv, TCI) in toluene (0.61 M). The resulting mixture was heated at 110° C. for 30 h. The reaction was then quenched with methanol and the volatiles were removed in vacuo. The resulting residue was partitioned between saturated aq. NaHCO₃ and EtOAc. The organic layer was separated, washed further with brine, dried over Na₂SO₄, filtered, and the filtrate concentrated in vacuo. The crude product thus obtained was subjected to reverse-phase column chromatography (C₁₈, gradient elution: 9:1 (v/v) H₂O: MeCN+0.1% formic acid→MeCN+0.1% formic acid) to afford the title compound as a white solid (49% yield). LCMS: m/z=472.0 [M+H]⁺; ¹H NMR (DMSO-d₆): δ=8.26 (d, J=8.1 Hz, 1H), 7.60˜7.49 (m, 2H), 7.46 (t, J=72.6 Hz, 1H), 7.39 (td, J=7.7, 1.7 Hz, 1H), 7.29 (dd, J=7.7, 1.2 Hz, 1H), 7.05 (d, J=8.1 Hz, 1H), 6.40 (s, 1H), 4.98 (p, J=8.4 Hz, 1H), 3.11 (d, J=8.0 Hz, 2H), 2.94 (sept, J=6.8 Hz, 1H), 2.39˜2.27 (m, 4H), 2.23˜1.83 (m, 4H), 1.19 (d, J=6.8 Hz, 3H), 1.10 (d, J=6.8 Hz, 3H).

The following example was prepared in an analogous fashion to Example 191, but substituting Example 27 with the requisite nitrile.

| | | | LCMS |
|---|---|---|---|
| Example | Starting Nitrile | Structure | (m/z) |
| 192 | 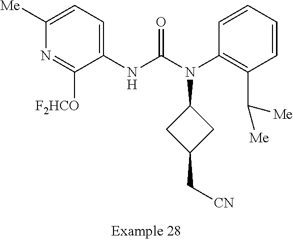 [SMILES: Cc1ccc(NC(=O)N(c2ccccc2C(C)C)[C@H]2C[C@@H](CC#N)C2)c(OC(F)F)n1] | 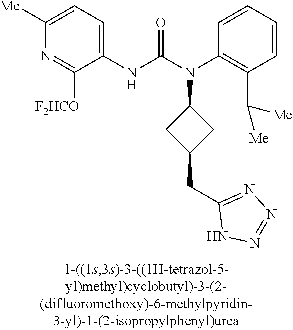 [SMILES: Cc1ccc(NC(=O)N(c2ccccc2C(C)C)[C@H]2C[C@@H](Cc3nnn[nH]3)C2)c(OC(F)F)n1] | 472.0 [M + H]⁺ |
| 248 | 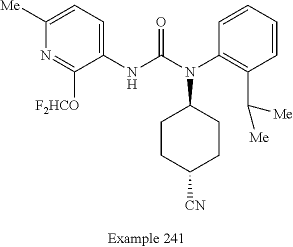 [SMILES: Cc1ccc(NC(=O)N(c2ccccc2C(C)C)[C@H]2CC[C@H](C#N)CC2)c(OC(F)F)n1] | 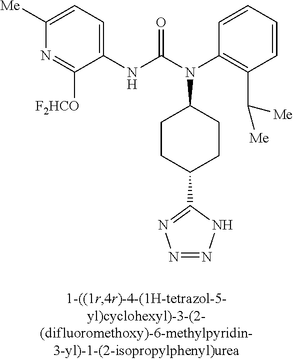 [SMILES: Cc1ccc(NC(=O)N(c2ccccc2C(C)C)[C@H]2CC[C@H](c3nnn[nH]3)CC2)c(OC(F)F)n1] | 485.8 [M + H]⁺ |

### Example 193: Preparation of 3-(2-(difluoromethoxy)-6-methylpyridin-3-yl)-l-((1 s,3s)-3-(hydroxymethyl)cyclobutyl)-1-(2-isopropylphenyl)urea

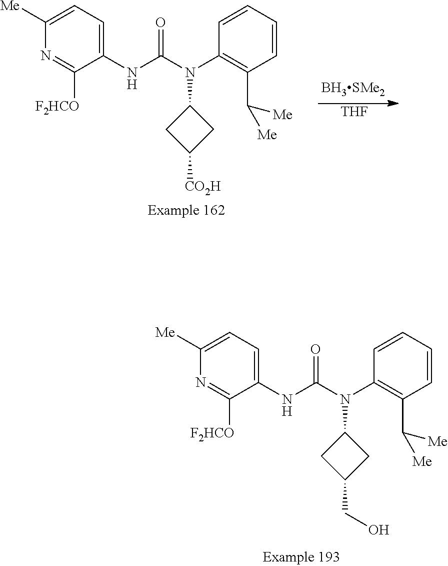 [SMILES: Cc1ccc(NC(=O)N(c2ccccc2C(C)C)[C@H]2C[C@@H](C(=O)O)C2)c(OC(F)F)n1 ; Cc1ccc(NC(=O)N(c2ccccc2C(C)C)[C@H]2C[C@@H](CO)C2)c(OC(F)F)n1 ; "BH3·SMe2" ; "THF"]

In a dried, round-bottom flask equipped with a magnetic stirrer was dissolved Example 162 (1 equiv) in THF (0.07 M). To this colorless solution was then added borane dimethyl sulfide complex (2 M solution in THF, 3 equiv, Sigma-Aldrich) and the resulting mixture was stirred at RT for 30 min. The reaction was then carefully quenched with HCl (1 M solution in water, 20 equiv) and extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: 4:1 (v/v) Hex: EtOAc→EtOAc) afforded the title compound as a white solid (69% yield). LCMS: m/z=420.1 [M+H]⁺.

The following example was prepared in an analogous fashion to Example 193, but substituting Example 162 with the requisite acid.

| | | | LCMS |
|---|---|---|---|
| Example | Starting Acid | Structure | (m/z) |
| 242 | 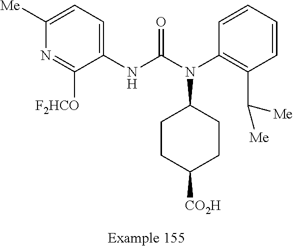 [SMILES: Cc1ccc(NC(=O)N(c2ccccc2C(C)C)[C@H]2CC[C@@H](C(=O)O)CC2)c(OC(F)F)n1] | 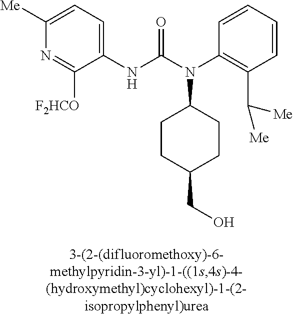 [SMILES: Cc1ccc(NC(=O)N(c2ccccc2C(C)C)[C@H]2CC[C@@H](CO)CC2)c(OC(F)F)n1] | 447.8 [M + H]⁺ |

### Example 194: Preparation of 3-(2-(difluoromethoxy)-6-methylpyridin-3-yl)-1-(3-formylcyclobutyl)-1-(2-isopropylphenyl)urea

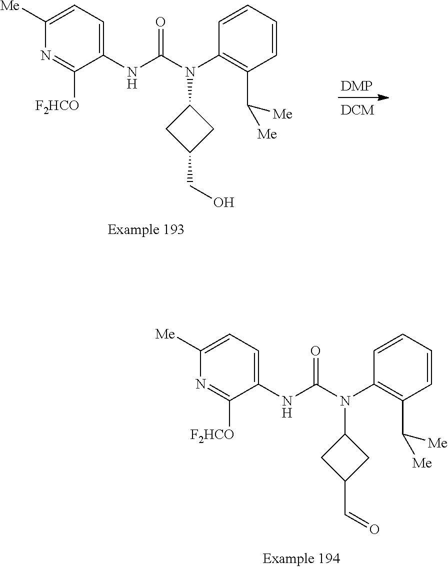 [SMILES: Cc1ccc(NC(=O)N(c2ccccc2C(C)C)[C@H]2C[C@@H](CO)C2)c(OC(F)F)n1 ; Cc1ccc(NC(=O)N(c2ccccc2C(C)C)C2CC(C=O)C2)c(OC(F)F)n1]

In a dried, round-bottom flask equipped with a magnetic stirrer was dissolved Example 193 (1 equiv) in dichloromethane (0.094 M). To this colorless solution was then added Dess-Martin periodinane (1.5 equiv, Sigma-Aldrich) in one rapid portion and the resulting mixture was stirred at RT for 30 min. The reaction was then quenched with saturated aq. NaHCO₃ and extracted with dichloromethane. The combined organic extracts were washed further with brine, dried over Na₂SO₄, filtered, and the filtrate concentrated in vacuo. Purification of the crude product thus obtained by way of column chromatography (SiO₂, gradient elution: Hex→1:1 (v/v) Hex: EtOAc) afforded the title compound as a white solid (57% yield). LCMS: m/z=418.1 [M+H]⁺. ¹H NMR (DMSO-d₆): δ=9.48˜9.47 (m, 1H), 8.26 (d, J=8.1 Hz, 0.5H, diastereomer A), 8.22 (d, J=8.1 Hz, 0.5H, diastereomer B), 7.61˜7.50 (m, 2H), 7.48 (t, J=72.6 Hz, 0.5H, diastereomer A), 7.46 (t, J=72.6 Hz, 0.5H, diastereomer B), 7.42˜7.24 (m, 2H), 7.06 (d, J=8.1 Hz, 0.5H, diastereomer A), 7.05 (d, J=8.1 Hz, 0.5H, diastereomer B), 6.54 (s, 0.5H, diastereomer A), 6.40 (s, 0.5H, diastereomer B), 4.88˜4.77 (m, 0.5H, diastereomer A), 4.73˜4.62 (m, 0.5H, diastereomer B), 3.26˜3.21 (m, 1H), 3.07˜2.90 (m, 2H), 2.40˜2.21 (m, 4H), 2.09˜1.79 (m, 2H), 1.21˜1.09 (m, 6H).

The following example was prepared in an analogous fashion to Example 194, but substituting Example 193 with the requisite alcohol.

| | | | LCMS |
|---|---|---|---|
| Example | Starting Alcohol | Structure | (m/z) |
| 195 | 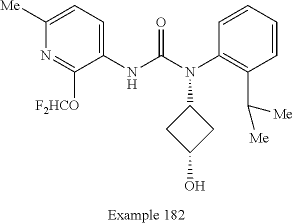 | 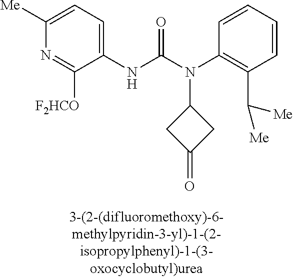 [SMILES: Cc1ccc(NC(=O)N(c2ccccc2C(C)C)C2CC(=O)C2)c(OC(F)F)n1] | 404.1 [M + H]⁺ |
| 243 | 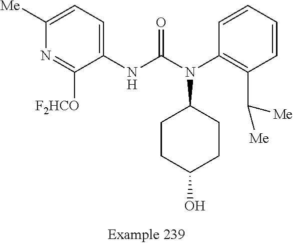 [SMILES: Cc1ccc(NC(=O)N(c2ccccc2C(C)C)[C@H]2CC[C@H](O)CC2)c(OC(F)F)n1] | 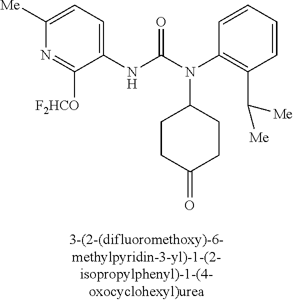 [SMILES: Cc1ccc(NC(=O)N(c2ccccc2C(C)C)C2CCC(=O)CC2)c(OC(F)F)n1] | 431.8 [M + H]⁺ |
| 244 | 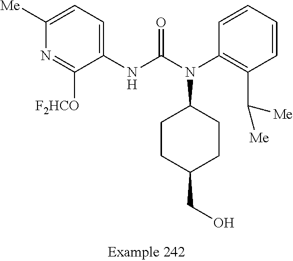 [SMILES: Cc1ccc(NC(=O)N(c2ccccc2C(C)C)[C@H]2CC[C@@H](CO)CC2)c(OC(F)F)n1] | 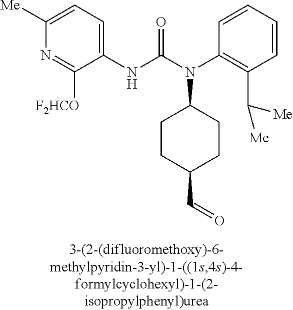 [SMILES: Cc1ccc(NC(=O)N(c2ccccc2C(C)C)[C@H]2CC[C@@H](C=O)CC2)c(OC(F)F)n1] | 445.8 [M + H]⁺ |

### Example 196: Preparation of 3-(2-(difluoromethoxy)-6-methylpyridin-3-yl)-1-(3-hydroxy-3-(trifluoromethyl)cyclobutyl)-1-(2-isopropylphenyl)urea

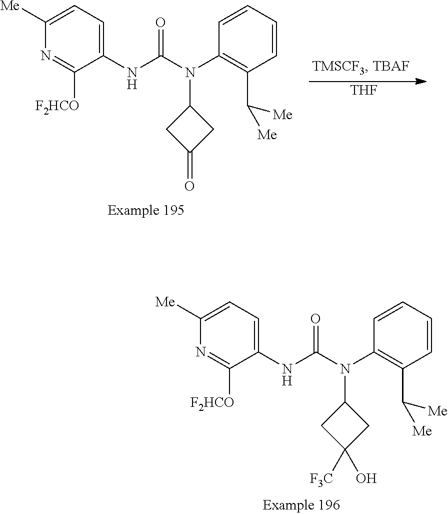 [SMILES: Cc1ccc(NC(=O)N(c2ccccc2C(C)C)C2CC(=O)C2)c(OC(F)F)n1 ; Cc1ccc(NC(=O)N(c2ccccc2C(C)C)C2CC(O)(C(F)(F)F)C2)c(OC(F)F)n1 ; "THF"]

In a dried, round-bottom flask equipped with a magnetic stirrer was combined Example 195 (1 equiv) and trimethyl(trifluoromethyl)silane (1.2 equiv, Sigma-Aldrich) in THF (0.062 M). To this colorless solution was then added, at 0° C., tetrabutylammonium fluoride (1 M solution in THF, 1 equiv, Sigma-Aldrich) dropwise over a period of 5 min. The resulting brown solution was first stirred at 0° C. for 15 min and then at RT for 1 h. The reaction was then carefully quenched with water and extracted with tert-butyl methyl ether. The combined organic extracts were washed further with brine, dried over Na₂SO₄, filtered, and the filtrate concentrated in vacuo. Purification of the crude product thus obtained by way of column chromatography (SiO₂, gradient elution: Hex→7:3 (v/v) Hex: EtOAc) afforded the title compound as a white solid (39% yield). LCMS: m/z=474.0 [M+H]⁺.

### Example 197: Preparation of 3-(3-(1-acetylpiperidin-4-yl)-3-(2-isopropylphenyl)ureido)-2-(difluoromethoxy)-6-methylpyridine 1-oxide

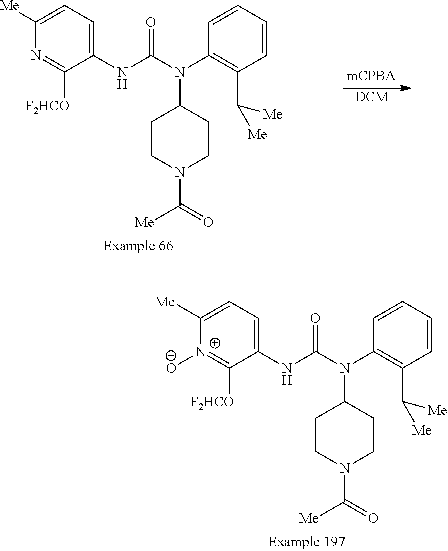

In a dried, round-bottom flask equipped with a magnetic stirrer was dissolved Example 66 (1 equiv) in dichloromethane (0.027 M). To this was then added m-chloroperoxybenzoic acid (1.5 equiv, Sigma-Aldrich) and the resulting mixture was stirred at RT for 16 h. The reaction was then quenched with water and extracted with DCM. The combined organic extracts were washed sequentially with water and brine, dried over Na₂SO₄, filtered, and the filtrate concentrated in vacuo. The crude product thus obtained was subjected to reverse-phase column chromatography (C₁s, gradient elution: 95:5 (v/v) H₂O: MeCN→5:95 (v/v) H₂O: MeCN) to afford the title compound as a white solid (4% yield). LCMS: m/z=477.1 [M+H]⁺.

### Example 198: Preparation of 1-(1-acetylpiperidin-4-yl)-3-(5-chloro-2-(difluoromethoxy)-6-methoxypyridin-3-yl)-1-(2-isopropylphenyl)urea

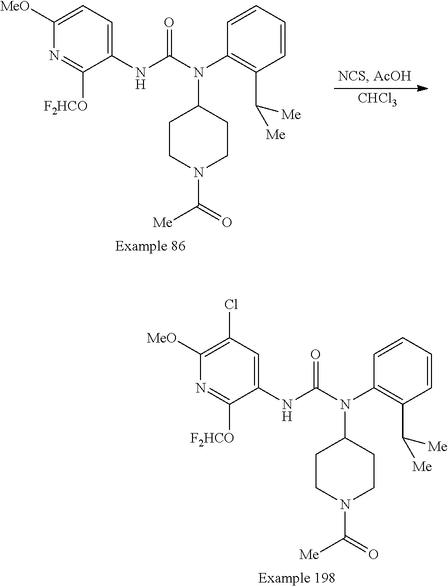 [SMILES: COc1ccc(NC(=O)N(c2ccccc2C(C)C)C2CCN(C(C)=O)CC2)c(OC(F)F)n1 ; "CHCl3" ; COc1nc(OC(F)F)c(NC(=O)N(c2ccccc2C(C)C)C2CCN(C(C)=O)CC2)cc1Cl]

In a dried, round-bottom flask equipped with a magnetic stirrer was dissolved Example 86 (1 equiv) in a 1:1 (v/v) solution of chloroform and acetic acid (0.021 M). To this was then added N-chlorosuccinimide (2.5 equiv, Sigma-Aldrich) and the resulting mixture was heated at 50° C. for 16 h. The reaction was then quenched with saturated aq. NaHCO₃ and extracted with EtOAc. The combined organic extracts were washed sequentially with water and brine, dried over Na₂SO₄, filtered, and the filtrate concentrated in vacuo. Purification of the crude product thus obtained by way of column chromatography (SiO₂, gradient elution: Hex→EtOAc) afforded the title compound as a white solid (33% yield). LCMS: m/z=511.0, 513.0 [M+H]⁺. ¹H NMR (CDCl₃): δ=8.64 (d, J=1.5 Hz, 1H), 7.53˜7.50 (m, 2H), 7.37˜7.30 (m, 1H), 7.13 (t, J=8.7 Hz, 1H), 7.12 (t, J=72.6 Hz, 1H), 6.01 (s, 1H), 4.70˜4.57 (m, 2H), 3.94˜3.82 (m, 4H), 3.30˜3.10 (m, 2H), 2.70˜2.57 (m, 1H), 2.22˜2.03 (m, 4H), 1.93˜1.80 (m, 1H), 1.70˜1.55 (m, 1H), 1.28˜1.15 (m, 7H).

### Example 199: Preparation of methyl 4-(3-(4-(difluoromethoxy)-6-methylpyridin-3-yl)-1-(2-isopropylphenyl)ureido)piperidine-1-carboxylate

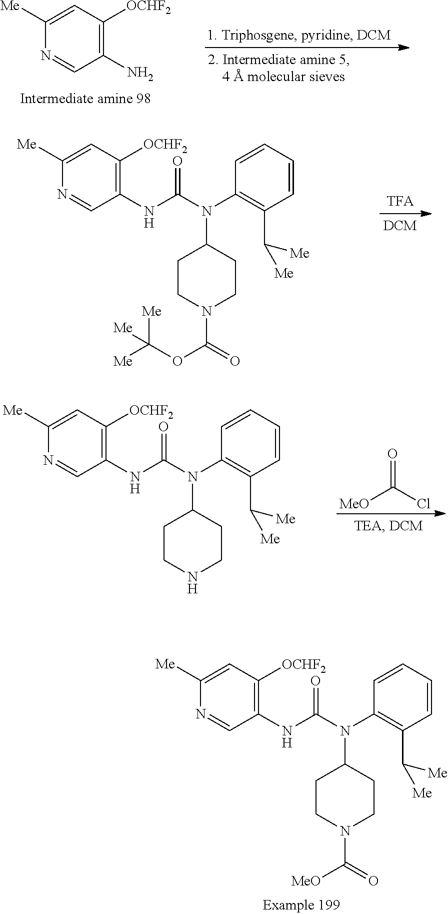 [SMILES: Cc1cc(OC(F)F)c(N)cn1 ; Cc1cc(OC(F)F)c(NC(=O)N(c2ccccc2C(C)C)C2CCN(C(=O)OC(C)(C)C)CC2)cn1 ; Cc1cc(OC(F)F)c(NC(=O)N(c2ccccc2C(C)C)C2CCNCC2)cn1 ; COC(=O)Cl ; COC(=O)N1CCC(N(C(=O)Nc2cnc(C)cc2OC(F)F)c2ccccc2C(C)C)CC1]

Step 1: In a dried, round-bottom flask equipped with a magnetic stirrer was combined Intermediate amine 98 (1 equiv) and pyridine (3 equiv, Sigma-Aldrich) in anhydrous dichloromethane (0.29 M). To this was then added triphosgene (0.5 equiv, Sigma-Aldrich) in one rapid portion at RT and the resulting solution was stirred at RT for 30 min. To this mixture was then added, at 0° C., Intermediate amine 5 (1 equiv) and freshly activated 4 Å molecular sieves. The resulting suspension was warmed to and stirred at RT for 2 h, before the reaction was quenched with water. The aqueous layer was separated and back extracted with DCM. The combined organic extracts were dried over Na₂SO₄, filtered, and the filtrate concentrated in vacuo. Purification of the crude product thus obtained by way of column chromatography (SiO₂, 4:1 (v/v) Hex: EtOAc) afforded tert-butyl 4-(3-(4-(difluoromethoxy)-6-methylpyridin-3-yl)-1-(2-isopropylphenyl)ureido)piperidine-1-carboxylate as a white solid (54% yield).

Step 2: In a round-bottom flask equipped with a magnetic stirrer was dissolved tert-butyl 4-(3-(4-(difluoromethoxy)-6-methylpyridin-3-yl)-1-(2-isopropylphenyl)ureido)piperidine-1-carboxylate (1 equiv) from the previous step in dichloromethane (0.14 M). To this solution was then added trifluoroacetic acid (150 equiv, Sigma-Aldrich) and the resulting reaction mixture was stirred at RT for 2 h. The volatiles were then removed in vacuo and the resulting residue was rendered slightly basic (pH˜8) with the addition of ammonia (7 M solution in MeOH, Sigma-Aldrich). The volatiles were once again removed in vacuo and the crude product thus obtained was purified further by way of reverse-phase column chromatography (C₁s, gradient elution: 9:1 (v/v) H₂O: MeCN→MeCN) to afford 3-(4-(difluoromethoxy)-6-methylpyridin-3-yl)-1-(2-isopropylphenyl)-1-(piperidin-4-yl)urea as a pink solid (88% yield).

Step 3: In a round-bottom flask equipped with a magnetic stirrer was combined 3-(4-(difluoromethoxy)-6-methylpyridin-3-yl)-1-(2-isopropylphenyl)-1-(piperidin-4-yl)urea (1 equiv) from the previous step and triethylamine (3 equiv, Sigma-Aldrich) in dichloromethane (0.045 M). To this was then added methyl chloroformate (2 equiv, Sigma-Aldrich), neat and dropwise, over a period of 5 min. After 1 h of stirring at RT, the volatiles were then removed in vacuo. Purification of the crude product thus obtained by way of reverse-phase column chromatography (C₁s, gradient elution: 9:1 (v/v) H₂O: MeCN→MeCN) afforded the title compound as a white solid (51% yield). LCMS: m/z=477.2 [M+H]⁺; ¹H NMR (methanol-d₄): δ=8.90 (s, 1H), 7.57˜7.48 (m, 2H), 7.38˜7.34 (m, 1H), 7.26˜7.24 (m, 1H), 7.02 (s, 1H), 6.89 (t, J=74.0 Hz, 1H), 4.47˜4.43 (m, 1H), 4.19˜4.11 (m, 2H), 3.63 (s, 3H), 3.20˜3.14 (m, 1H), 2.90 (br s, 2H), 2.46 (s, 3H), 2.09 (br d, J=12.8 Hz, 1H), 1.83 (br d, J=12.8 Hz, 1H), 1.61˜1.57 (m, 1H), 1.29˜1.17 (m, 7H).

The following example was prepared in an analogous fashion to Example 199, but substituting methyl chloroformate with the requisite electrophile.

| | Starting | | LCMS |
|---|---|---|---|
| Example | Electrophile | Structure | (m/z) |
| 200 | 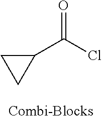 | 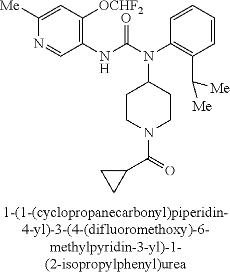 [SMILES: Cc1cc(OC(F)F)c(NC(=O)N(c2ccccc2C(C)C)C2CCN(C(=O)C3CC3)CC2)cn1] | 487.3 [M + H]⁺ |

### Example 201: Preparation of 3-(4-(difluoromethoxy)-6-methylpyridin-3-yl)-1-(2-isopropylphenyl)-1-(1-(oxetan-3-yl)piperidin-4-yl)urea

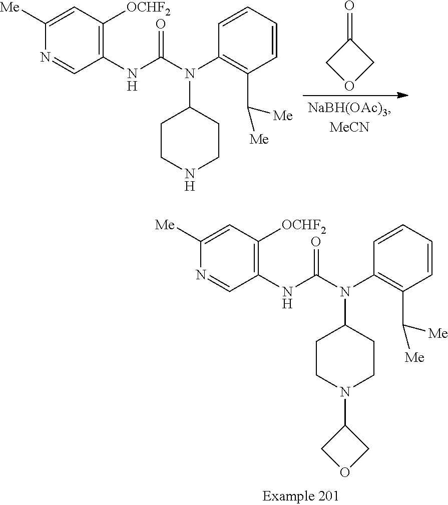 [SMILES: Cc1cc(OC(F)F)c(NC(=O)N(c2ccccc2C(C)C)C2CCNCC2)cn1 ; O=C1COC1 ; Cc1cc(OC(F)F)c(NC(=O)N(c2ccccc2C(C)C)C2CCN(C3COC3)CC2)cn1]

In a round-bottom flask equipped with a magnetic stirrer was combined 3-(4-(difluoromethoxy)-6-methylpyridin-3-yl)-1-(2-isopropylphenyl)-1-(piperidin-4-yl)urea (1 equiv, Example 199, Step 2) and oxetan-3-one (1.5 equiv, Combi-Blocks) in acetonitrile (0.048 M). To this solution was then added sodium triacetoxyborohydride (2 equiv, Sigma-Aldrich) portionwise and the resulting suspension was stirred at RT for 1 h. The reaction suspension was then filtered through a bed of celite and the insolubles were rinsed further with EtOAc. The combined filtrate was then concentrated in vacuo and the crude product thus obtained was purified further by way of reverse-phase column chromatography (C₁s, gradient elution: 9:1 (v/v) H₂O: MeCN→MeCN) to afford the title compound as a white solid (45% yield). LCMS: m/z=475.2 [M+H]⁺; ¹H NMR (methanol-d₄): δ=8.91 (s, 1H), 7.58˜7.51 (m, 2H), 7.39˜7.35 (m, 1H), 7.29˜7.27 (m, 1H), 7.02 (s, 1H), 6.89 (t, J=72.4 Hz, 1H), 4.63 (q, J=6.4 Hz, 2H), 4.52 (q, J=6.4 Hz, 2H), 4.39˜4.32 (m, 1H), 3.48˜3.42 (m, 1H), 3.19 (sept, J=6.4 Hz, 1H), 2.85˜2.76 (m, 2H), 2.45 (s, 3H), 2.09˜1.64 (m, 5H), 1.41˜1.32 (m, 1H), 1.30 (d, J=6.4 Hz, 3H), 1.17 (d, J=6.4 Hz, 3H).

### Example 202: Preparation of 3-(4-(difluoromethoxy)-6-methylpyridin-3-yl)-1-(2-isopropylphenyl)-1-(1-(3-methyloxetan-3-yl)piperidin-4-yl)urea

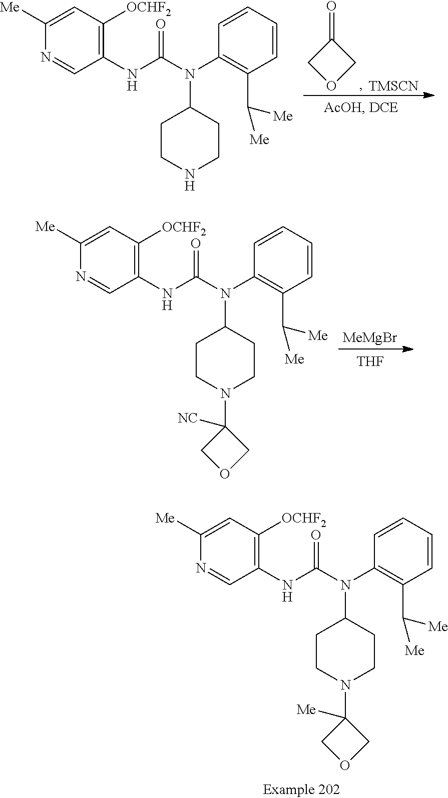

Step 1: In a round-bottom flask equipped with a magnetic stirrer was combined 3-(4-(difluoromethoxy)-6-methylpyridin-3-yl)-1-(2-isopropylphenyl)-1-(piperidin-4-yl)urea (1 equiv, Example 199, Step 2), oxetan-3-one (1.5 equiv, Combi-Blocks), and glacial acetic acid (1.2 equiv, Fisher Scientific) in 1,2-dichloroethane (0.29 M). The resulting solution was heated at 50° C. for 30 min, cooled to RT, and then added trimethylsilyl cyanide (2 equiv, Sigma-Aldrich) dropwise at RT. The resulting mixture was then stirred at RT for an additional 12 h before the reaction was carefully quenched with water. The aqueous layer was then separated and back extracted with dichloromethane. The combined organic extracts were washed further with brine, dried over Na₂SO₄, filtered, and the filtrate concentrated in vacuo. Purification of the crude product thus obtained by way of column chromatography (SiO₂, gradient elution, Hex→EtOAc) afforded 1-(1-(3-cyanooxetan-3-yl)piperidin-4-yl)-3-(4-(difluoromethoxy)-6-methylpyridin-3-yl)-1-(2-isopropylphenyl)urea as a yellow oil (77% yield).

Step 2: In a dried round-bottom flask equipped with a magnetic stirrer was dissolved 1-(1-(3-cyanooxetan-3-yl)piperidin-4-yl)-3-(4-(difluoromethoxy)-6-methylpyridin-3-yl)-1-(2-isopropylphenyl)urea (1 equiv) from the previous step in THE (0.027 M). To this solution was then added, at 0° C., methylmagnesium bromide (3 M solution in diethyl ether, 6 equiv, Sigma-Aldrich) dropwise over a period of 5 min. Following the completion of addition, the reaction mixture was heated at 60° C. for 3.5 h. After cooling to 0° C., the reaction was carefully quenched with saturated aq. NH₄Cl and then extracted with EtOAc. The combined organic extracts were washed further with water and brine, dried over Na₂SO₄, filtered, and the filtrate concentrated in vacuo. The crude product thus obtained was purified further by way of reverse-phase column chromatography (C₁₈, gradient elution: 9:1 (v/v) H₂O: MeCN→MeCN) to afford the title compound as a white solid (31% yield). LCMS: m/z=489.3 [M+H]⁺; ¹H NMR (methanol-d₄): δ=8.94 (s, 1H), 7.60˜7.53 (m, 2H), 7.41˜7.37 (m, 1H), 7.31˜7.28 (m, 1H), 7.03 (s, 1H), 6.90 (t, J=72.4 Hz, 1H), 4.54˜4.49 (m, 2H), 4.39˜4.32 (m, 1H), 4.22˜4.18 (m, 2H), 3.19 (sept, J=6.8 Hz, 1H), 2.65˜2.56 (m, 2H), 2.47 (s, 3H), 2.32˜2.26 (m, 2H), 2.13˜2.09 (m, 1H), 1.86˜1.70 (m, 2H), 1.39˜1.30 (m, 7H), 1.18 (d, J=6.8 Hz, 3H).

The following examples were prepared via chiral chromatographic separation of the corresponding mixture of stereoisomers.

| | | | RT |
|---|---|---|---|
| Example | Starting Mixture | Separation Conditions | (min) |
| 203 204 | 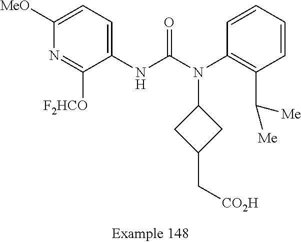 [SMILES: COc1ccc(NC(=O)N(c2ccccc2C(C)C)C2CC(CC(=O)O)C2)c(OC(F)F)n1] | Column: CHIRALPAK IE (2 × 25 cm; 5 μm) Mobile phase: 93:7 (v/v) Hex: EtOH Additive: 0.1% formic acid Flow rate: 20 mL/min Loading solvent: 0.4 mL EtOH | 23.00 25.42 |
| 205 206 | 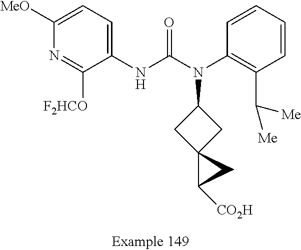 [SMILES: COc1ccc(NC(=O)N(c2ccccc2C(C)C)[C@H]2C[C@@]3(C[C@@H]3C(=O)O)C2)c(OC(F)F)n1] | Column: Lux Celluose-2 (2 × 25 cm; 5 μm) Mobile phase: 90:10 (v/v) Hex: EtOH Additive: 0.1% TFA Flow rate: 20 mL/min Loading solvent: 1.2 mL EtOH | 9.65 21.56 |
| 207 208 | 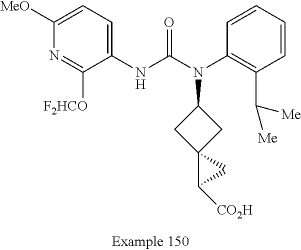 [SMILES: COc1ccc(NC(=O)N(c2ccccc2C(C)C)[C@H]2C[C@]3(C[C@H]3C(=O)O)C2)c(OC(F)F)n1] | Column: CHIRALPAK IG (2 × 25 cm; 5 μm) Mobile phase: 90:10 (v/v) Hex: ⁱPrOH Additive: 0.1% formic acid Flow rate: 20 mL/min Loading solvent: 0.8 mL EtOH | 11.93 18.70 |
| 209 210 | 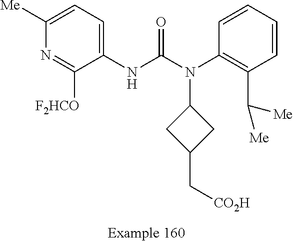 [SMILES: Cc1ccc(NC(=O)N(c2ccccc2C(C)C)C2CC(CC(=O)O)C2)c(OC(F)F)n1] | Column: CHIRALPAK IE (2 × 25 cm; 5 μm) Mobile phase: 93:7 (v/v) Hex: ⁱPrOH Additive: 0.1% formic acid Flow rate: 20 mL/min Loading solvent: 0.5 mL EtOH | 17.00 19.84 |
| 211 212 | 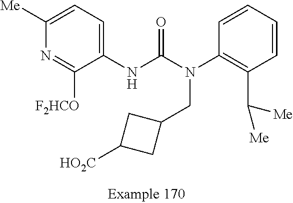 [SMILES: Cc1ccc(NC(=O)N(CC2CC(C(=O)O)C2)c2ccccc2C(C)C)c(OC(F)F)n1] | Column: Lux i-Celluose (3 × 25 cm; 5 μm) Mobile phase: 80:20 (v/v) supercritical CO₂: ⁱPrOH Additive: 0.1% 2 M NH₃ in MeOH Flow rate: 60 mL/min Loading solvent: 1 mL 1:1 (v/v) MeOH: ⁱPrOH | 7.44 8.46 |
| 213 214 | 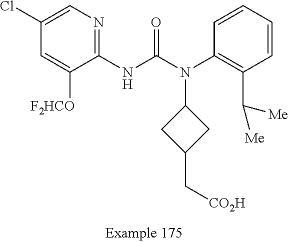 [SMILES: CC(C)c1ccccc1N(C(=O)Nc1ncc(Cl)cc1OC(F)F)C1CC(CC(=O)O)C1] | Column: CHIRALPAK ID (2 × 25 cm; 5 μm) Mobile phase: 90:10 (v/v) Hex: iPrOH Additive: 0.1% formic acid Flow rate: 20 mL/min Loading solvent: 1 mL EtOH | 18.03 27.53 |
| 215 216 | 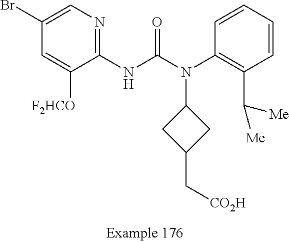 [SMILES: CC(C)c1ccccc1N(C(=O)Nc1ncc(Br)cc1OC(F)F)C1CC(CC(=O)O)C1] | Column: CHIRALPAK ID (2 × 25 cm; 5 μm) Mobile phase: 90:10 (v/v) Hex: ⁱPrOH Additive: 0.1% formic acid Flow rate: 20 mL/min Loading solvent: 1 mL EtOH | 19.69 29.01 |
| 290 291 | 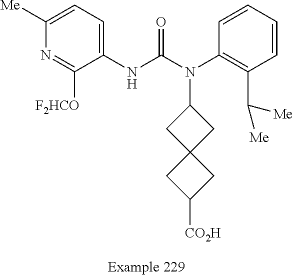 [SMILES: Cc1ccc(NC(=O)N(c2ccccc2C(C)C)C2CC3(CC(C(=O)O)C3)C2)c(OC(F)F)n1] | Column: LUX CELLULOSE-4 (2 × 25 cm; 5 μm) Mobile phase: 95:5 (v/v) Hex: EtOH Additive: 0.1% formic acid Flow rate: 20 mL/min Loading solvent: 0.5 mL EtOH | 11.78 16.65 |
| 292 293 | 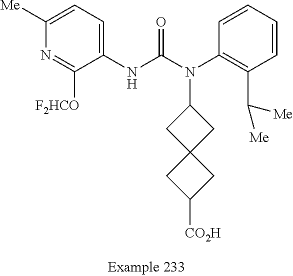 [SMILES: Cc1ccc(NC(=O)N(c2ccccc2C(C)C)C2CC3(CC(C(=O)O)C3)C2)c(OC(F)F)n1] | Column: LUX CELLULOSE-2 (2 × 25 cm; 5 μm) Mobile phase: 95:5 (v/v) Hex: ⁱPrOH Additive: 0.1% TFA Flow rate: 20 mL/min Loading solvent: 0.5 mL EtOH | 11.40 17.40 |

### Example 218: Preparation of 1-(1-acetylpiperidin-4-yl)-3-(2-(difluoromethoxy)-5-fluoro-6-methylpyridin-3-yl)-1-(2-isopropylphenyl)urea

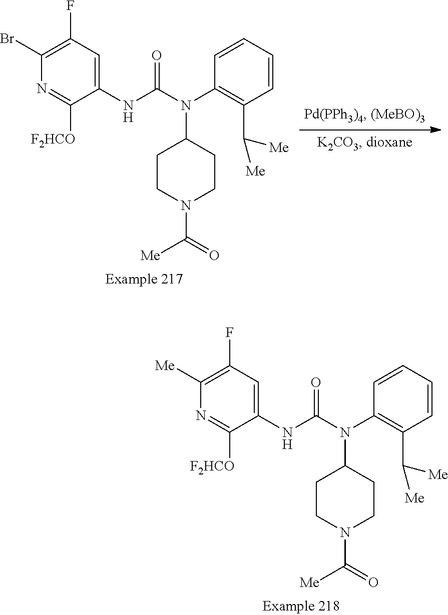 [SMILES: CC(=O)N1CCC(N(C(=O)Nc2cc(F)c(Br)nc2OC(F)F)c2ccccc2C(C)C)CC1 ; CC(=O)N1CCC(N(C(=O)Nc2cc(F)c(C)nc2OC(F)F)c2ccccc2C(C)C)CC1]

In a thick-walled, glass reaction vessel equipped with a magnetic stirrer and a Teflon screwcap was combined Example 217 (1 equiv), potassium carbonate (2 equiv, Sigma-Aldrich), and tetrakis(triphenylphosphine)palladium(0) (0.1 equiv, Sigma-Aldrich) in 1,4-dioxane (0.031 M). The resulting suspension was deoxygenated via subsurface purging with nitrogen for 10 min before trimethylboroxine (4 equiv, Sigma-Aldrich) was added neat and dropwise to the reaction suspension. The reaction vessel was then tightly sealed and heated at 100° C. for 3 h. After cooling to RT, the volatiles were then removed in vacuo and the residue thus obtained was subjected to reverse-phase column chromatography (C₁s, gradient elution: 9:1 (v/v) H₂O: MeCN+0.1% formic acid→MeCN+0.1% formic acid) to afford the title compound as a white solid (54% yield). LCMS: m/z=479.0 [M+H]⁺; ¹H NMR (DMSO-d₆): δ=8.31 (d, J=10.9 Hz, 1H), 7.65˜7.50 (m, 2H), 7.39 (t, J=72.1 Hz, 1H), 7.36 (d, J=7.2 Hz, 1H), 7.27 (dd, J=7.2, 1.3 Hz, 1H), 6.37 (s, 1H), 4.49˜4.39 (m, 2H), 3.90˜3.80 (m, 1H), 3.16˜3.06 (m, 2H), 2.60˜2.56 (m, 3H), 2.28 (d, J=2.9 Hz, 3H), 2.07˜1.99 (m, 1H), 1.95 (s, 1.5H, rotamer A), 1.92 (s, 1.5H, rotamer B), 1.81˜1.73 (m, 1H), 1.21˜1.06 (m, 6H).

### Example 221: Preparation of (1r,4r)-4-(3-(2-(difluoromethoxy)-5-fluoro-6-methylpyridin-3-yl)-1-(2-isopropylphenyl)ureido)cyclohexane-1-carboxylic acid

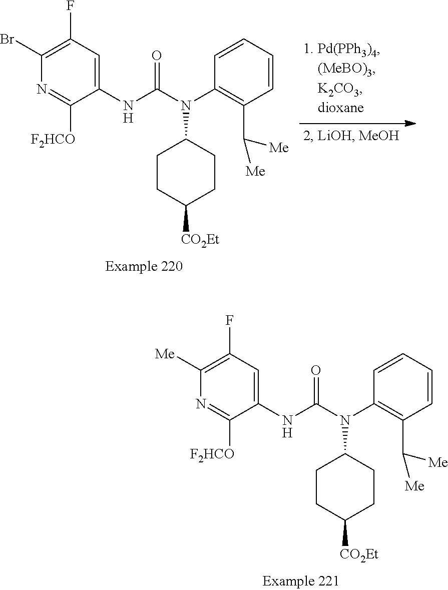 [SMILES: CCOC(=O)[C@H]1CC[C@H](N(C(=O)Nc2cc(F)c(Br)nc2OC(F)F)c2ccccc2C(C)C)CC1 ; CCOC(=O)[C@H]1CC[C@H](N(C(=O)Nc2cc(F)c(C)nc2OC(F)F)c2ccccc2C(C)C)CC1]

Step 1: In a thick-walled, glass reaction vessel equipped with a magnetic stirrer and a Teflon screwcap was combined Example 220 (1 equiv), potassium carbonate (2 equiv, Sigma-Aldrich), and tetrakis(triphenylphosphine)palladium(0) (0.1 equiv, Sigma-Aldrich) in 1,4-dioxane (0.027 M). The resulting suspension was deoxygenated via subsurface purging with nitrogen for 10 min before trimethylboroxine (4 equiv, Sigma-Aldrich) was added neat and dropwise to the reaction suspension. The reaction vessel was then tightly sealed and heated at 110° C. for 16 h. After cooling to RT, the volatiles were then removed in vacuo and the residue thus obtained was subjected to reverse-phase column chromatography (C₁₈, gradient elution: 9:1 (v/v) H₂O: MeCN+0.1% formic acid→MeCN+0.1% formic acid) to afford ethyl (1r,4r)-4-(3-(2-(difluoromethoxy)-5-fluoro-6-methylpyridin-3-yl)-1-(2-isopropylphenyl)ureido)cyclohexane-1-carboxylate as a white solid (89% yield).

Step 2: In a round-bottom flask equipped with a magnetic stirrer was dissolved ethyl (1r,4r)-4-(3-(2-(difluoromethoxy)-5-fluoro-6-methylpyridin-3-yl)-1-(2-isopropylphenyl)ureido)cyclohexane-1-carboxylate (1 equiv) from the previous step in a 1:1 (v/v) solution of methanol and THE (0.059 M). To this colorless solution was then added lithium hydroxide (2 M solution in water, 5 equiv) and the resulting mixture was heated at 60° C. for 10 min. Upon cooling to RT, the reaction mixture was carefully quenched with HCl (1 M solution in water, 5 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded the title compound as a white solid (88% yield). LCMS: m/z=480.0 [M+H]⁺; ¹H NMR (DMSO-d₆): δ=12.04 (s, 1H), 8.34 (d, J=10.9 Hz, 1H), 7.60˜7.49 (m, 2H), 7.39 (t, J=72.3 Hz, 1H), 7.36 (dd, J=7.1, 1.4 Hz, 1H), 7.27 (dd, J=7.8, 1.4 Hz, 1H), 6.33 (s, 1H), 4.26˜4.15 (m, 1H), 3.08 (sept, J=6.6 Hz, 1H), 2.28 (d, J=2.9 Hz, 3H), 2.10˜1.80 (m, 6H), 1.50˜1.36 (m, 3H), 1.24˜1.06 (m, 6H).

The following examples were prepared in an analogous fashion to Example 221, but substituting Example 220 with the requisite starting bromide. In Example 223 and Example 232, trimethylboroxine (4 equiv) in step 1 was also substituted with cyclopropylboronic acid (3 equiv, Combi-Blocks).

| | | | LCMS |
|---|---|---|---|
| Example | Starting Bromide | Structure | (m/z) |
| 222 | 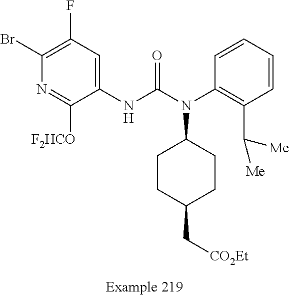 [SMILES: CCOC(=O)C[C@H]1CC[C@@H](N(C(=O)Nc2cc(F)c(Br)nc2OC(F)F)c2ccccc2C(C)C)CC1] | 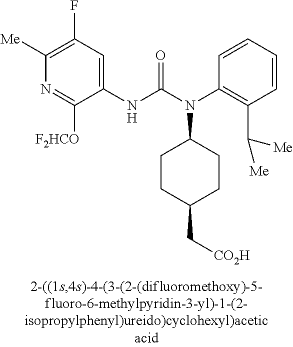 [SMILES: Cc1nc(OC(F)F)c(NC(=O)N(c2ccccc2C(C)C)[C@H]2CC[C@@H](CC(=O)O)CC2)cc1F] | 494.0 [M + H]⁺ |
| 223 | 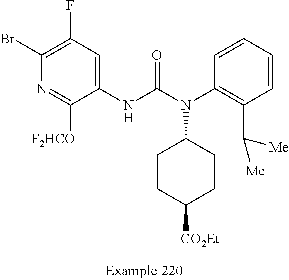 [SMILES: CCOC(=O)[C@H]1CC[C@H](N(C(=O)Nc2cc(F)c(Br)nc2OC(F)F)c2ccccc2C(C)C)CC1] | 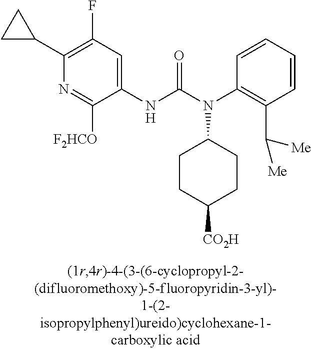 [SMILES: CC(C)c1ccccc1N(C(=O)Nc1cc(F)c(C2CC2)nc1OC(F)F)[C@H]1CC[C@H](C(=O)O)CC1] | 506.0 [M + H]⁺ |
| 232 | 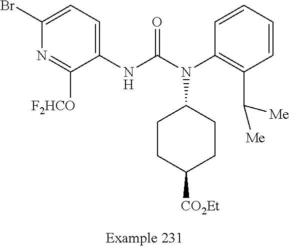 [SMILES: CCOC(=O)[C@H]1CC[C@H](N(C(=O)Nc2ccc(Br)nc2OC(F)F)c2ccccc2C(C)C)CC1] | 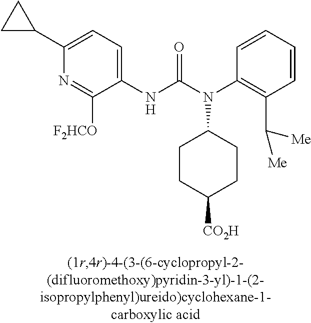 [SMILES: CC(C)c1ccccc1N(C(=O)Nc1ccc(C2CC2)nc1OC(F)F)[C@H]1CC[C@H](C(=O)O)CC1] | 488.1 [M + H]⁺ |

### Example 225: Preparation of 1-(1-acetylpiperidin-4-yl)-3-(2-(difluoromethoxy)-5-fluoro-6-methoxypyridin-3-yl)-l-(2-isopropylphenyl)urea

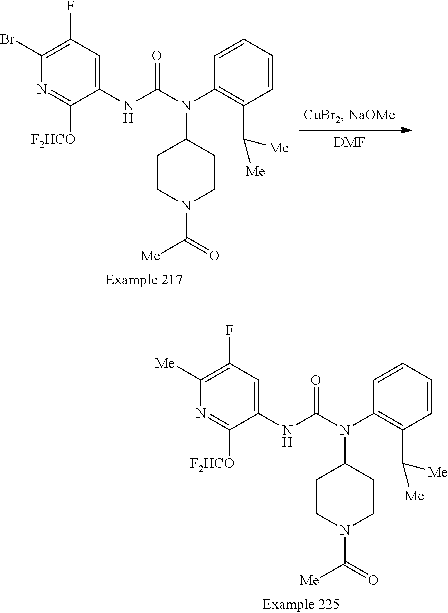

In a thick-walled, glass reaction vessel equipped with a magnetic stirrer and a Teflon screwcap was combined Example 217 (1 equiv) and copper(II) bromide (2 equiv, Sigma-Aldrich) in DMF (0.13 M). The resulting suspension was deoxygenated via subsurface purging with nitrogen for 10 min before sodium methoxide (5.4 M solution in methanol, 5 equiv, Sigma-Aldrich) was added dropwise to the reaction suspension over 5 min. The reaction vessel was then tightly sealed and heated at 100° C. for 1 h. After cooling to RT, the reaction mixture was diluted with water and EtOAc. The aqueous layer was separated and back extracted with EtOAc. The combined organic extracts were washed further with saturated aq. NaHCO₃ and brine, dried over Na₂SO₄, filtered, and the filtrate concentrated in vacuo. The crude product thus obtained was subjected to column chromatography (SiO₂, gradient elution: Hex→1: (v/v) Hex: EtOAc) to afford the title compound as a white solid (7% yield). LCMS: m/z=495.0 [M+H]⁺.

### Example 245: Preparation of 3-(2-(difluoromethoxy)-6-methylpyridin-3-yl)-1-((1s,4s)-4-(difluoromethyl)cyclohexyl)-1-(2-isopropylphenyl)urea

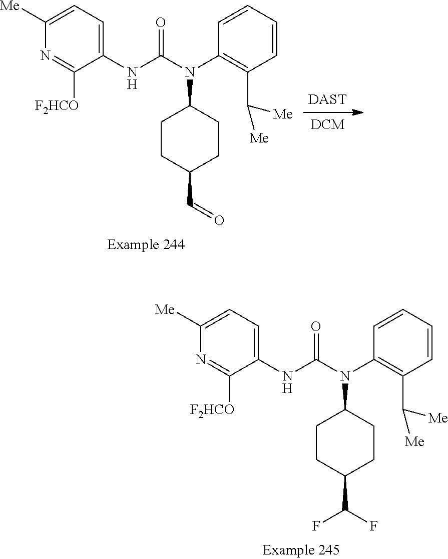

In a Nalgene reaction vessel equipped with a magnetic stirrer was dissolved Example 244 (1 equiv) in dichloromethane (0.063 M). To this colorless solution was then added, at 0° C., diethylaminosulfur trifluoride (0.5 M solution in dichloromethane, 1.5 equiv, Sigma-Aldrich) dropwise over a period of 5 min. The resulting mixture was then allowed to warm slowly to RT over 16 h. After cooling again to 0° C., the reaction was carefully quenched with saturated aq. NaHCO₃ and extracted with EtOAc. The combined organic extracts were washed further with water and brine, dried over Na₂SO₄, filtered, and the filtrate concentrated in vacuo. The crude product thus obtained was subjected to column chromatography (SiO₂, gradient elution: Hex→9:1 (v/v) Hex: EtOAc) to afford the title compound as a white solid (13% yield). LCMS: m/z=467.8 [M+H]⁺; ¹H NMR (CDCl₃): δ=8.47 (d, J=8.1 Hz, 1H), 7.63˜7.30 (m, 4H), 7.25 (t, J=72.9 Hz, 1H), 7.17 (d, J=7.4 Hz, 1H), 6.89 (d, J=8.1 Hz, 1H), 6.23 (s, 1H), 5.79 (td, J=57.0, 6.4 Hz, 1H), 4.40˜4.31 (m, 1H), 3.21 (sept, J=6.8 Hz, 1H), 2.35 (s, 3H), 2.10˜1.94 (m, 3H), 1.81˜1.64 (m, 4H), 1.41˜1.30 (m, 1H), 1.26 (d, J=6.8 Hz, 3H), 1.15 (d, J=6.8 Hz, 3H),

### Example 247: Preparation of 3-(2-(difluoromethoxy)-6-methylpyridin-3-yl)-1-((1r,4r)-4-(2-hydroxy-2-methylpropyl)cyclohexyl)-1-(2-isopropylphenyl)urea

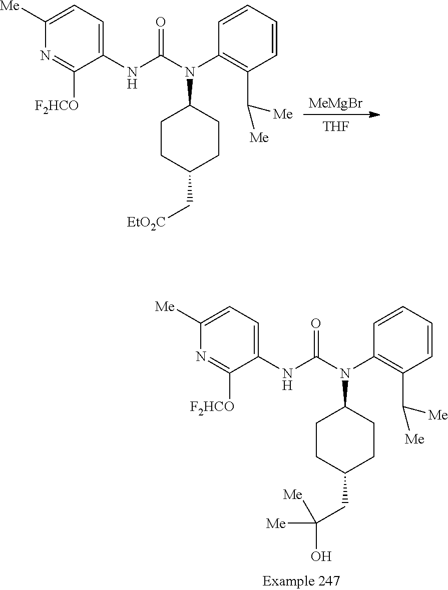 [SMILES: "MeMgBr" ; CCOC(=O)C[C@H]1CC[C@H](N(C(=O)Nc2ccc(C)nc2OC(F)F)c2ccccc2C(C)C)CC1 ; Cc1ccc(NC(=O)N(c2ccccc2C(C)C)[C@H]2CC[C@H](CC(C)(C)O)CC2)c(OC(F)F)n1 ; "THF"]

In a dried, round-bottom flask equipped with a magnetic stirrer was dissolved ethyl 2-((1r,4r)-4-(3-(2-(difluoromethoxy)-6-methylpyridin-3-yl)-1-(2-isopropylphenyl)ureido)cyclohexyl)acetate (1 equiv, Example 165, Step 1) in THF (0.12 M). To this solution was added, at 0° C., methylmagnesium bromide (3 M solution in diethyl ether, 3.3 equiv, Sigma-Aldrich) dropwise over a period of 5 min and the resulting suspension was stirred first at 0° C. for 30 min and then at RT for 3 h. The reaction was then carefully quenched with the sequential addition of water and saturated aq. NH₄Cl. The resulting suspension was vigorously stirred at RT for 30 min, and then extracted with EtOAc. The combined organic extracts were dried over MgSO₄, filtered, and the filtrate concentrated in vacuo. Purification of the crude product thus obtained by way of column chromatography (SiO₂, gradient elution: Hex→4:1 (v/v) Hex: EtOAc) afforded the title compound as a white solid (77% yield). LCMS: m/z=489.9 [M+H]⁺; ¹H NMR (DMSO-d₆): δ=8.34 (d, J=8.1 Hz, 1H), 7.57 (dd, J=7.9, 1.7 Hz, 1H), 7.50 (td, J=7.9, 1.4 Hz, 1H), 7.44 (t, J=72.6 Hz, 1H), 7.36 (td, J=7.9, 1.7 Hz, 1H), 7.25 (dd, J=7.9, 1.4 Hz, 1H), 7.04 (d, J=8.1 Hz, 1H), 6.26 (s, 1H), 4.24˜4.16 (m, 1H), 4.00 (s, 1H), 3.09 (sept, J=6.7 Hz, 1H), 2.31 (s, 3H), 2.02˜1.96 (m, 1H), 1.89˜1.79 (m, 2H), 1.73˜1.70 (m, 1H), 1.29˜1.04 (m, 19H),

### Example 276: Preparation of 3-(2-(difluoromethoxy)-6-methoxypyridin-3-yl)-1-(2-isopropylphenyl)-1-(4-oxocyclohexyl)urea

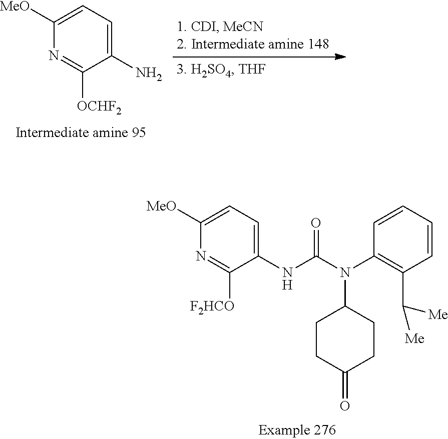 [SMILES: COc1ccc(N)c(OC(F)F)n1 ; COc1ccc(NC(=O)N(c2ccccc2C(C)C)C2CCC(=O)CC2)c(OC(F)F)n1]

Step 1: In a dried, round-bottom flask equipped with a magnetic stirrer was dissolved Intermediate amine 95 (1 equiv) in anhydrous acetonitrile (0.15 M). To this was added 1,1′-carbonyldiimidazole (1.5 equiv, Sigma-Aldrich) in one rapid portion and the resulting solution was stirred at RT for 30 min. To this mixture was then added Intermediate amine 148 (1 equiv) and the resulting purple solution was heated at 60° C. for 36 h. After cooling to RT, the reaction was quenched with water. The volatiles were then removed in vacuo and the resulting aqueous suspension was back extracted with EtOAc. The combined organic extracts were dried over Na₂SO₄, filtered, and the filtrate concentrated in vacuo. Purification of the crude product thus obtained by way of column chromatography (SiO₂, gradient elution: Hex→2:1 (v/v) Hex: EtOAc) afforded 3-(2-(difluoromethoxy)-6-methoxypyridin-3-yl)-1-(2-isopropylphenyl)-1-(1,4-dioxaspiro[4.5]decan-8-yl)urea as a tan solid (64% yield).

Step 2: In a glass round-bottom flask equipped with a magnetic stirrer was dissolved 3-(2-(difluoromethoxy)-6-methoxypyridin-3-yl)-1-(2-isopropylphenyl)-1-(1,4-dioxaspiro[4.5]decan-8-yl)urea (1 equiv) from the previous step in THE (0.094 M). To this solution was then added sulfuric acid (2 M solution in water, 10 equiv) dropwise over a period of 5 min and the resulting solution was stirred first at RT for 30 min and then at 50° C. for 2 h. After cooling to RT, the reaction mixture was diluted with water and extracted with EtOAc. The combined organic extracts were then washed further with saturated aq. NaHCO₃ and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded the title compound as a white solid (89% yield). LCMS: m/z=448.1 [M+H]⁺; ¹H NMR (CDCl₃): δ=8.41 (d, J=8.7 Hz, 1H), 7.50˜7.45 (m, 2H), 7.34˜7.28 (m, 1H), 7.18 (t, J=72.9 Hz, 1H), 7.15 (dd, J=8.4, 0.9 Hz, 1H), 6.52 (d, J=8.7 Hz, 1H), 5.99 (s, 1H), 4.86 (tt, J=12.1, 3.6 Hz, 1H), 3.80 (s, 3H), 3.22 (sept, J=6.8 Hz, 1H), 2.62˜2.34 (m, 5H), 2.19˜2.10 (m, 1H), 1.98˜1.83 (m, 1H), 1.62˜1.48 (m, 1H), 1.25 (d, J=6.8 Hz, 3H), 1.17 (d, J=6.8 Hz, 3H),

### Example 278: Preparation of (1r,4r)-4-(3-(2-(difluoromethoxy)-6-methoxypyridin-3-yl)-1-(2-(2-fluoropropan-2-yl)phenyl)ureido)cyclohexane-1-carboxylic acid

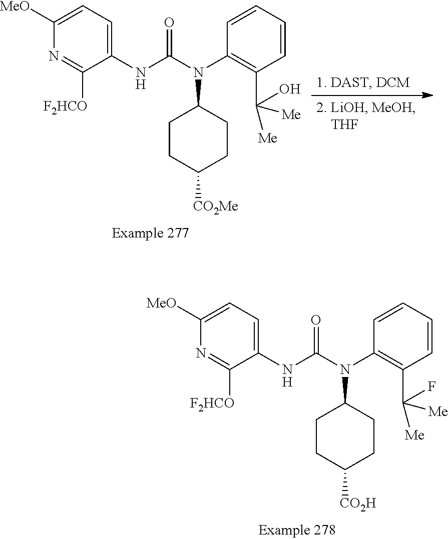 [SMILES: COc1ccc(NC(=O)N(c2ccccc2C(C)(C)O)[C@H]2CC[C@H](C(=O)OC)CC2)c(OC(F)F)n1 ; COc1ccc(NC(=O)N(c2ccccc2C(C)(C)F)[C@H]2CC[C@H](C(=O)O)CC2)c(OC(F)F)n1]

In a Nalgene reaction vessel equipped with a magnetic stirrer was dissolved Example 277 (1 equiv) in dichloromethane (0.040 M). To this colorless solution was then added, at −78° C., diethylaminosulfur trifluoride (1 M solution in dichloromethane, 1.3 equiv, Sigma-Aldrich) dropwise over a period of 5 min. After 20 min of stirring at −78° C., the reaction mixture was diluted further with dichloromethane and then carefully quenched with water. The organic layer was separated, dried over Na₂SO₄, filtered, and the filtrate concentrated in vacuo. The crude product thus obtained was subjected to column chromatography (SiO₂, gradient elution: Hex→2:1 (v/v) Hex: EtOAc) to afford crude methyl (1r,4r)-4-(3-(2-(difluoromethoxy)-6-methoxypyridin-3-yl)-1-(2-(2-fluoropropan-2-yl)phenyl)ureido)cyclohexane-1-carboxylate as a viscous oil. This was immediately taken up in a 2:1 (v/v) solution of methanol and THF, and then lithium hydroxide (2 M solution in water, 6 equiv) was added. The resulting mixture was heated at 50° C. for 2 h. Upon cooling to RT, the reaction mixture was carefully quenched with HCl (1 M solution in water, 6 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded the title compound as a white solid (61% yield). LCMS: m/z=496.0 [M+H]⁺; ¹H NMR (CDCl₃): δ=8.42 (d, J=8.7 Hz, 1H), 7.50˜7.38 (m, 3H), 7.21 (d, J=7.4 Hz, 1H), 7.19 (t, J=73.0 Hz, 1H), 6.50 (d, J=8.7 Hz, 1H), 5.88 (s, 1H), 3.97 (tt, J=11.6, 3.5 Hz, 1H), 3.79 (s, 3H), 2.43˜2.39 (m, 1H), 2.28˜2.18 (m, 1H), 2.13˜1.95 (m, 3H), 1.77 (d, J=26.4 Hz, 3H), 1.69 (d, J=26.4 Hz, 3H), 1.67˜1.48 (m, 4H).

### Example 279: Preparation of (1r,4r)-4-(3-(2-(difluoromethoxy)-6-methoxypyridin-3-yl)-1-(2-isopropylphenyl)ureido)-N-(methylsulfonyl)cyclohexane-1-carboxamide

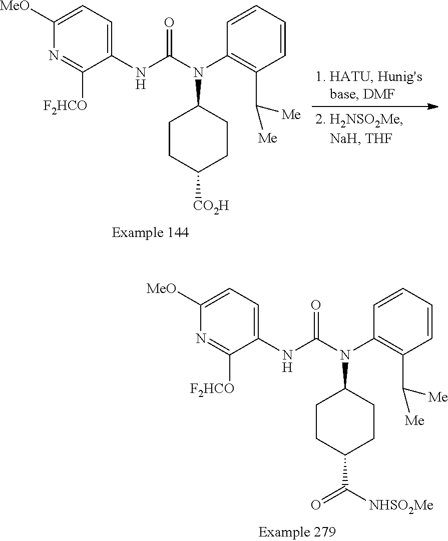

In a dried, round-bottom flask equipped with a magnetic stirrer was combined Example 144 (1 equiv) and N,N-diisopropylethylamine (3 equiv, Sigma-Aldrich) in anhydrous DMF (0.067 M). To this colorless solution was then added HATU (1.2 equiv, Chem-Impex) in one rapid portion and the resulting solution was stirred at RT for 30 min. Concurrently, in a separate dried, round-bottom flask equipped with a magnetic stirrer was combined methanesulfonamide (5 equiv, Sigma-Aldrich) and sodium hydride (60% w/w dispersion in paraffin oil, 4 equiv, Sigma-Aldrich) in THE (0.25 M). To this THE solution was then added dropwise the DMF solution of HATU-activated carboxylic acid over a period of 5 min. After the completion of addition, the reaction mixture was allowed to stir at RT for a further 30 min. The reaction mixture was then diluted further with EtOAc and quenched with saturated aq. NH₄Cl. The organic layer was separated and washed further with water and brine, dried over Na₂SO₄, filtered, and the filtrate concentrated in vacuo. The crude product thus obtained was subjected to reverse-phase column chromatography (C₁₈, gradient elution: 9:1 (v/v) H₂O: MeCN+0.1% formic acid→MeCN+0.1% formic acid) to afford the title compound as a white solid (66% yield). LCMS: m/z=554.8 [M+H]⁺; ¹H NMR (DMSO-d₆): δ=11.54 (1H), 8.15 (d, J=8.6 Hz, 1H), 7.56˜7.45 (m, 2H), 7.53 (t, J=72.5 Hz, 1H), 7.33 (td, J=7.8, 1.6 Hz, 1H), 7.23 (d, J=6.8 Hz, 1H), 6.62 (d, J=8.6 Hz, 1H), 6.27 (s, 1H), 4.14 (tt, J=11.6, 3.5 Hz, 1H), 3.77 (s, 3H), 3.20 (s, 3H), 3.13 (sept, J=6.8 Hz, 1H), 2.12˜2.04 (m, 2H), 1.90˜1.82 (m, 3H), 1.50˜1.33 (m, 3H), 1.21 (d, J=6.8 Hz, 3H), 1.12 (d, J=6.8 Hz, 3H), 1.09˜0.98 (m, 1H).

The following example was prepared in an analogous fashion to Example 279, but substituting methanesulfonamide with the requisite nucleophile. In Example 281, sodium hydride (60% w/w dispersion in paraffin oil, 4 equiv) was also substituted with N,N-diisopropylethylamine (10 equiv, Sigma-Aldrich).

| | Starting | | LCMS |
|---|---|---|---|
| Example | Nucleophile | Structure | (m/z) |
| 280 | 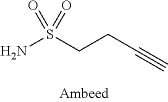 | 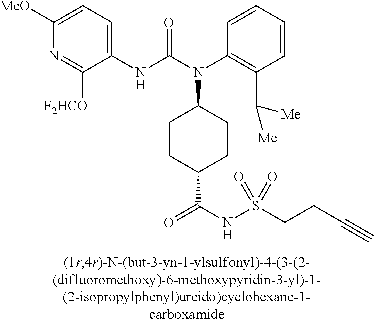 [SMILES: C#CCCS(=O)(=O)NC(=O)[C@H]1CC[C@H](N(C(=O)Nc2ccc(OC)nc2OC(F)F)c2ccccc2C(C)C)CC1] | 593.1 [M + H]⁺ |
| 281 | NH₂OH HCl Sigma-Aldrich | 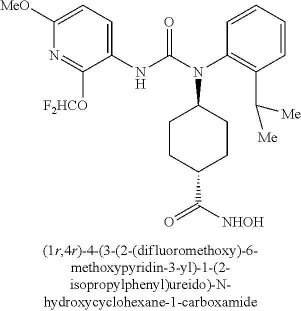 | 492.8 [M + H]⁺ |

### Example 282: Preparation of (1r,4r)-4-(3-(2-(difluoromethoxy)-6-methoxypyridin-3-yl)-l-(2-(1-hydroxypropan-2-yl)phenyl)ureido)cyclohexane-1-carboxylic acid

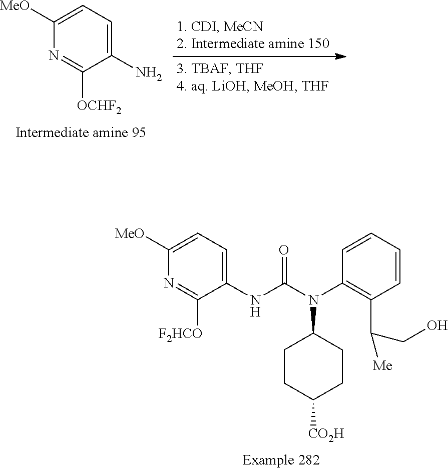 [SMILES: COc1ccc(N)c(OC(F)F)n1 ; COc1ccc(NC(=O)N(c2ccccc2C(C)CO)[C@H]2CC[C@H](C(=O)O)CC2)c(OC(F)F)n1]

Step 1: In a dried, round-bottom flask equipped with a magnetic stirrer was dissolved Intermediate amine 95 (1 equiv) in anhydrous acetonitrile (0.08 M). To this was then added 1,1′-carbonyldiimidazole (1.8 equiv, Sigma-Aldrich) in one rapid portion, and the resulting solution was stirred at RT for 1 h. To this mixture was then added Intermediate amine 150 (1 equiv) and the resulting purple solution was heated at 70° C. for 24 h. After cooling to RT, the reaction was quenched with water. The volatiles were then removed in vacuo and the resulting aqueous suspension was back extracted with EtOAc. The combined organic extracts were dried over Na₂SO₄, filtered, and the filtrate concentrated in vacuo. Purification of the crude product thus obtained by way of column chromatography (SiO₂, gradient elution: Hex→4:1 (v/v) Hex: EtOAc) afforded methyl (1r,4r)-4-(1-(2-(1-((tert-butyldimethylsilyl)oxy)propan-2-yl)phenyl)-3-(2-(difluoromethoxy)-6-methoxypyridin-3-yl)ureido)cyclohexane-1-carboxylate as a colorless oil (45% yield).

Step 2: In a glass round-bottom flask equipped with a magnetic stirrer was dissolved methyl (1r,4r)-4-(1-(2-(1-((tert-butyldimethylsilyl)oxy)propan-2-yl)phenyl)-3-(2-(difluoromethoxy)-6-methoxypyridin-3-yl)ureido)cyclohexane-1-carboxylate (1 equiv) from the previous step in THE (0.024 M). To this solution was then added tetrabutylammonium fluoride (1 M solution in THF, 1.2 equiv, Sigma-Aldrich) dropwise over a period of 1 min, and the resulting mixture was stirred at RT for 30 min. The volatiles were then removed in vacuo and the resulting residue was directly subjected to purification by way of column chromatography (SiO₂, gradient elution: Hex→1:1 (v/v) Hex: EtOAc) to afford methyl (1r,4r)-4-(3-(2-(difluoromethoxy)-6-methoxypyridin-3-yl)-1-(2-(1-hydroxypropan-2-yl)phenyl)ureido)cyclohexane-1-carboxylate as a white solid (83% yield).

Step 3: In a glass round-bottom flask equipped with a magnetic stirrer was dissolved methyl (1r,4r)-4-(3-(2-(difluoromethoxy)-6-methoxypyridin-3-yl)-1-(2-(1-hydroxypropan-2-yl)phenyl)ureido)cyclohexane-1-carboxylate (1 equiv) from the previous step in a 2:1 (v/v) solution of methanol and THE (0.012 M). To this colorless solution was then added lithium hydroxide (1 M solution in water, 9 equiv) and the resulting mixture was heated at 45° C. for 6 h. Upon cooling to RT, the reaction mixture was carefully quenched with HCl (1 M solution in water, 9 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded the title compound as a 1.8:1 mixture of diastereomers (90% yield). LCMS: m/z=494.0 [M+H]⁺; ¹H NMR (CDCl₃): δ=8.41 (d, J=8.7 Hz, 0.36H, diastereomer A), 8.25 (d, J=8.7 Hz, 0.64H, diastereomer B), 7.51˜7.40 (m, 2H), 7.38˜7.21 (m, 2H), 7.19 (t, J=73.1 Hz, 0.64H, diastereomer B), 7.17 (t, J=73.1 Hz, 0.36H, diastereomer A), 6.50 (d, J=8.4 Hz, 0.36H, diastereomer A), 6.48 (d, J=8.7 Hz, 0.64H, diastereomer B), 6.26 (s, 0.64H, diastereomer B), 5.90 (s, 0.36H, diastereomer A), 4.30˜4.16 (m, 1H), 3.79˜3.63 (m, 5H), 3.43˜3.29 (m, 1H), 2.22˜1.96 (m, 5H), 1.65˜1.50 (m, 3H), 1.39˜1.16 (m, 5H).

### Example 283: Preparation of 3-(2-(difluoromethoxy)-6-methoxypyridin-3-yl)-1-((1r,4r)-4-(3-hydroxyisoxazol-5-yl)cyclohexyl)-1-(2-isopropylphenyl)urea

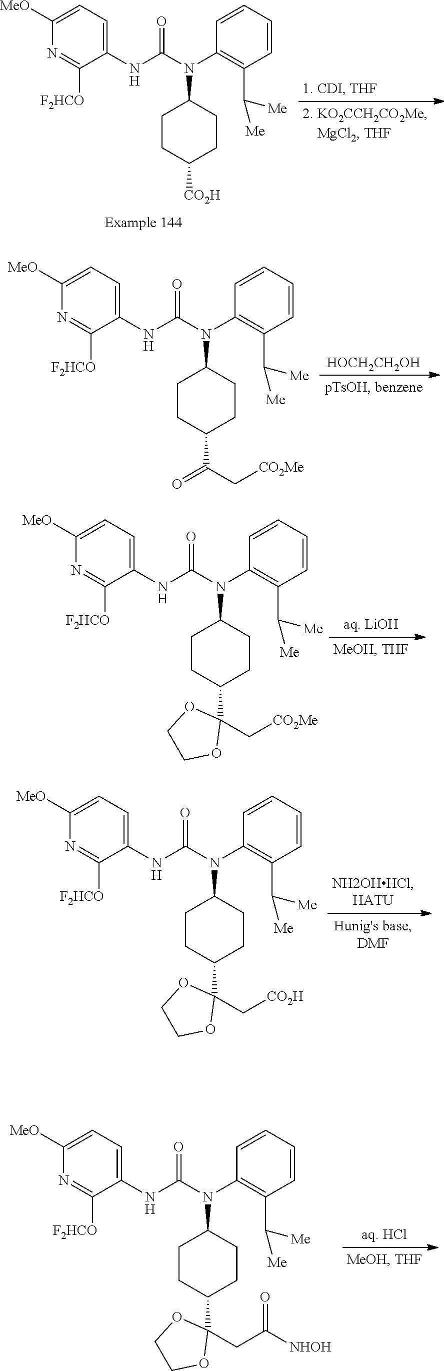

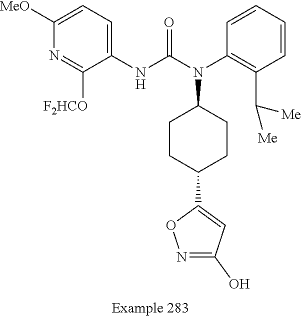 [SMILES: COc1ccc(NC(=O)N(c2ccccc2C(C)C)[C@H]2CC[C@H](c3cc(O)no3)CC2)c(OC(F)F)n1]

Step 1: In a dried, round-bottom flask equipped with a magnetic stirrer was dissolved Example 144 (1 equiv) in anhydrous THF (0.13 M). To this was then added 1,1′-carbonyldiimidazole (1.5 equiv, Sigma-Aldrich) in one rapid portion, and the resulting solution was stirred at RT for 1 h. To this mixture was then added, sequentially, methyl potassium malonate (1 equiv, Sigma-Aldrich) and magnesium chloride (1 equiv, Sigma-Aldrich) and the resulting suspension was heated at 45° C. for 16 h. After cooling to RT, the reaction was quenched with 1 M aq. HCl and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex→3:2 (v/v) Hex: EtOAc) afforded methyl 3-((1r,4r)-4-(3-(2-(difluoromethoxy)-6-methoxypyridin-3-yl)-1-(2-isopropylphenyl)ureido)cyclohexyl)-3-oxopropanoate as a white solid (85% yield).

Step 2: In a dried, round-bottom flask equipped with a magnetic stirrer was combined methyl 3-((1r,4r)-4-(3-(2-(difluoromethoxy)-6-methoxypyridin-3-yl)-1-(2-isopropylphenyl)ureido)cyclohexyl)-3-oxopropanoate (1 equiv) from the previous step, ethylene glycol (5 equiv, Fisher Scientific), and p-toluenesulfonic acid (a few crystals, Combi-Blocks) in benzene (0.019 M). A Dean-Stark trap was attached, and the resulting mixture was heated to reflux for 18 h. After cooling to RT, the reaction mixture diluted with dichloromethane and washed with water. The organic extract thus obtained was then dried over MgSO₄, filtered, and the filtrate concentrated in vacuo. Purification of the crude product thus obtained by way of column chromatography (SiO₂, gradient elution: Hex→2:3 (v/v) Hex: EtOAc) afforded methyl 2-(2-((1r,4r)-4-(3-(2-(difluoromethoxy)-6-methoxypyridin-3-yl)-1-(2-isopropylphenyl)ureido)cyclohexyl)-1,3-dioxolan-2-yl)acetate as a white solid (65% yield).

Step 3: In a glass round-bottom flask equipped with a magnetic stirrer was dissolved methyl 2-(2-((1r,4r)-4-(3-(2-(difluoromethoxy)-6-methoxypyridin-3-yl)-1-(2-isopropylphenyl)ureido)cyclohexyl)-1,3-dioxolan-2-yl)acetate (1 equiv) from the previous step in a 4:1 (v/v) solution of methanol and THF (0.026 M). To this colorless solution was then added lithium hydroxide (1 M solution in water, 6 equiv) and the resulting mixture was heated at 50° C. for 3 h. Upon cooling to RT, the reaction mixture was carefully quenched with HCl (1 M solution in water, 7 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded 2-(2-((1r,4r)-4-(3-(2-(difluoromethoxy)-6-methoxypyridin-3-yl)-1-(2-isopropylphenyl)ureido)cyclohexyl)-1,3-dioxolan-2-yl)acetic acid as a white solid (99% yield).

Step 4: In a glass round-bottom flask equipped with a magnetic stirrer was combined 2-(2-((1r,4r)-4-(3-(2-(difluoromethoxy)-6-methoxypyridin-3-yl)-1-(2-isopropylphenyl)ureido)cyclohexyl)-1,3-dioxolan-2-yl)acetic acid (1 equiv) from the previous step, hydroxylamine hydrochloride (1.5 equiv, Sigma-Aldrich), and N,N-diisopropylethylamine (5 equiv, Sigma-Aldrich) in DMF (0.044 M). To this solution was then added, at 0° C., HATU (2 equiv, Chem-Impex) in one rapid portion and the resulting yellow solution mixture was stirred at RT for 2 min. The reaction was then diluted with water and extracted with EtOAc. The combined organic extracts were washed further with brine, dried over Na₂SO₄, filtered, and the filtrate concentrated in vacuo. Purification of the crude product thus obtained by way of reverse-phase column chromatography (C₁₈, gradient elution: 9:1 (v/v) H₂O: MeCN+0.1% formic acid→MeCN+0.1% formic acid) afforded 2-(2-((1r,4r)-4-(3-(2-(difluoromethoxy)-6-methoxypyridin-3-yl)-1-(2-isopropylphenyl)ureido)cyclohexyl)-1,3-dioxolan-2-yl)-N-hydroxyacetamide as a white solid (44% yield).

Step 5: In a glass round-bottom flask equipped with a magnetic stirrer was heated concentrated HCl (400 equiv, Alfa Aesar) to 60° C. To this was then slowly added, at 60° C., 2-(2-((1r,4r)-4-(3-(2-(difluoromethoxy)-6-methoxypyridin-3-yl)-1-(2-isopropylphenyl)ureido)cyclohexyl)-1,3-dioxolan-2-yl)-N-hydroxyacetamide (1 equiv) from the previous step as a 2:1:1 (v/v/v) solution of THF, methanol and water (0.094 M) over a period of 5 min. After the completion of addition, the reaction vessel was quickly cooled to RT and diluted further with water and EtOAc. The organic layer was separated, dried over MgSO₄, filtered, and the filtrate concentrated in vacuo. Purification of the crude product thus obtained by way of column chromatography (SiO₂, gradient elution: Hex→1:4 (v/v) Hex: EtOAc) afforded the title compound as a white solid (63% yield). LCMS: m/z=517.1 [M+H]⁺; ¹H NMR (DMSO-d₆): δ=11.01 (s, 1H), 8.17 (d, J=8.6 Hz, 1H), 7.55 (dd, J=7.9, 1.4 Hz, 1H), 7.53 (t, J=72.5 Hz, 1H), 7.50˜7.45 (m, 1H), 7.34 (td, J=7.9, 1.7 Hz, 1H), 7.25 (dd, J=7.9, 1.4 Hz, 1H), 6.62 (d, J=8.6 Hz, 1H), 6.26 (s, 1H), 5.71 (s, 1H), 4.29˜4.20 (m, 1H), 3.77 (s, 3H), 3.14 (sept, J=6.7 Hz, 1H), 2.13˜1.82 (m, 4H), 1.57˜1.42 (m, 4H), 1.22 (d, J=6.7 Hz, 3H), 1.20˜1.16 (m, 1H), 1.12 (d, J=6.7 Hz, 3H),

### Example 297: Preparation of (1r,4r)-4-(3-(5-chloropyridin-2-yl)-1-(2-isopropylphenyl)ureido)cyclohexane-1-carboxylic acid

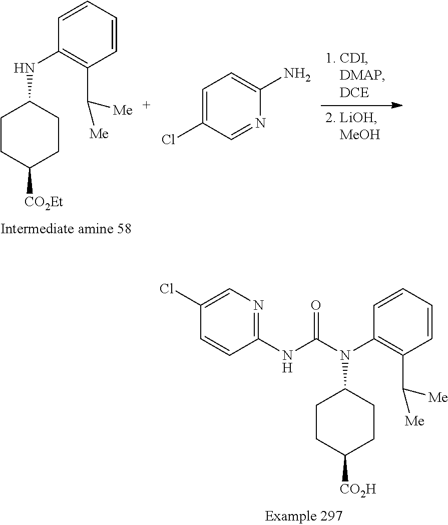 [SMILES: CCOC(=O)[C@H]1CC[C@H](Nc2ccccc2C(C)C)CC1 ; Nc1ccc(Cl)cn1 ; CC(C)c1ccccc1N(C(=O)Nc1ccc(Cl)cn1)[C@H]1CC[C@H](C(=O)O)CC1]

Step 1: In a dried, round-bottom flask equipped with a magnetic stirrer was combined Intermediate amine 58 (1 equiv), 2-amino-5-chloropyridine (1 equiv, Combi-Blocks), 1,1′-carbonyldiimidazole (1.5 equiv, Sigma-Aldrich), and 4-(dimethylamino)pyridine (1 equiv, Sigma-Aldrich) in anhydrous 1,2-dichloroethane (0.031 M). The resulting solution was heated at 85° C. for 2 h. The volatiles were then removed in vacuo and the resulting residue was partitioned between water and EtOAc. The organic layer was separated, washed further with brine, dried over Na₂SO₄, filtered, and the filtrate concentrated in vacuo. Purification of the crude product thus obtained by way of column chromatography (SiO₂, gradient elution: Hex→1:1 (v/v) Hex: EtOAc) afforded ethyl (1r,4r)-4-(3-(5-chloropyridin-2-yl)-1-(2-isopropylphenyl)ureido)cyclohexane-1-carboxylate as a yellow oil (36% yield).

Step 2: In a round-bottom flask equipped with a magnetic stirrer was dissolved ethyl (1r,4r)-4-(3-(5-chloropyridin-2-yl)-1-(2-isopropylphenyl)ureido)cyclohexane-1-carboxylate (1 equiv) from the previous step in a 2:1 (v/v) solution of THF and methanol (0.016 M). To this colorless solution was then added lithium hydroxide (1 M solution in water, 6 equiv) and the resulting mixture was stirred at RT for 1 h. The reaction mixture was then carefully quenched with HCl (1 M solution in water, 7 equiv) and the volatiles were removed in vacuo. The crude product thus obtained was subjected to reverse-phase column chromatography (C₁₈, gradient elution: 9:1 (v/v) H₂O: MeCN+0.1% formic acid→MeCN+0.1% formic acid) to afford the title compound as a white solid (59% yield). LCMS: m/z=416.2, 418.2 [M+H]⁺; ¹H NMR (methanol-d₄): δ=8.05 (d, J=9.2, Hz, 1H), 8.04 (s, 1H), 7.74 (dd, J=9.2, 2.8 Hz, 1H), 7.58˜7.52 (m, 2H), 7.40˜7.36 (m, 1H), 7.27 (d, J=7.6 Hz, 1H), 4.42˜4.32 (m, 1H), 3.15 (sept, J=6.8 Hz, 1H), 2.19˜2.16 (m, 1H), 2.05˜1.85 (m, 4H), 1.62˜1.52 (m, 3H), 1.28 (d, J=6.8 Hz, 3H), 1.13 (d, J=6.8 Hz, 3H), 1.11˜1.07 (m, 1H).

The following example was prepared in an analogous fashion to Example 297, but substituting Intermediate amine 58 with the requisite amine.

| | | | LCMS |
|---|---|---|---|
| Example | Starting Amine | Structure | (m/z) |
| 306 | 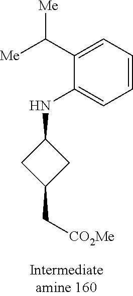 [SMILES: COC(=O)C[C@H]1C[C@@H](Nc2ccccc2C(C)C)C1] | 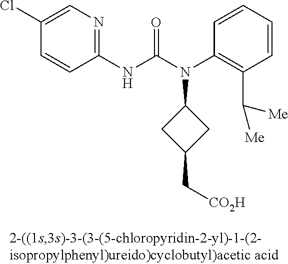 [SMILES: CC(C)c1ccccc1N(C(=O)Nc1ccc(Cl)cn1)[C@H]1C[C@@H](CC(=O)O)C1] | 402.2, 404.2 [M + H]⁺ |

### Example 321 & Example 322: Preparation of 2-((1r,4r)-4-(3-(2-(difluoromethoxy)-6-methoxypyridin-3-yl)-1-(2-isopropylphenyl)ureido)-1-hydroxycyclohexyl)-2,2-difluoroacetic acid and 2-((1s,4s)-4-(3-(2-(difluoromethoxy)-6-methoxypyridin-3-yl)-1-(2-isopropylphenyl)ureido)-1-hydroxycyclohexyl)-2,2-difluoroacetic acid

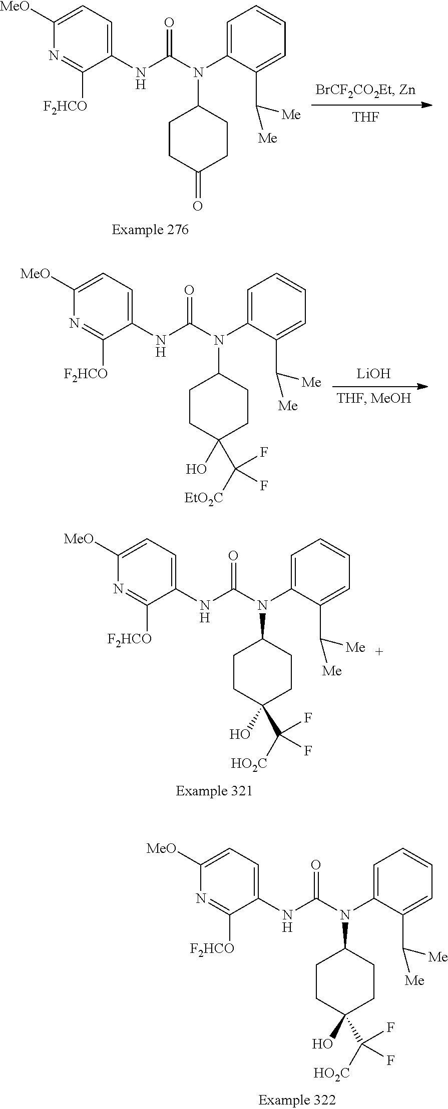

Step 1: In a dried, round-bottom flask equipped with a magnetic stirrer was combined Example 276 (1 equiv), ethyl 2-bromo-2,2-difluoroacetate (20 equiv, Sigma-Aldrich), and zinc (10 equiv, Strem) in THF (0.073 M). The resulting suspension was sonicated under nitrogen for 3 h. The reaction was then quenched with saturated aq. NH₄Cl and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 2:1 (v/v) Hex: EtOAc) afforded ethyl 2-(4-(3-(2-(difluoromethoxy)-6-methoxypyridin-3-yl)-1-(2-isopropylphenyl)ureido)-1-hydroxycyclohexyl)-2,2-difluoroacetate as an inseparable mixture of diastereomers (62% yield).

Step 2: In a dried, round-bottom flask equipped with a magnetic stirrer was dissolved ethyl 2-(4-(3-(2-(difluoromethoxy)-6-methoxypyridin-3-yl)-1-(2-isopropylphenyl)ureido)-1-hydroxycyclohexyl)-2,2-difluoroacetate (1 equiv) from the previous step in a 3:1 (v/v) solution of methanol and THE (0.053 M). To this colorless solution was then added lithium hydroxide (0.25 M solution in water, 9 equiv) and the resulting mixture was stirred at RT for 1 h. Finally, the reaction mixture was carefully quenched with HCl (1 M solution in water, 9 equiv) and extracted with EtOAc. The combined organic extracts were washed sequentially with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compounds, both as white solids. Example 321: faster eluting isomer; 22% yield; LCMS: m/z=544.2 [M+H]⁺; ¹H NMR (methanol-d₄): δ=8.09 (d, J=8.7 Hz, 1H), 7.56˜7.45 (m, 2H), 7.37˜7.24 (m, 2H), 7.35 (t, J=73.6 Hz, 1H), 6.53 (d, J=8.7 Hz, 1H), 4.38˜4.30 (m, 1H), 3.81 (s, 3H), 3.25 (sept, J=6.9 Hz, 1H), 1.94˜1.92 (m, 3H), 1.87˜1.69 (m, 4H), 1.51˜1.40 (m, 1H), 1.29 (d, J=6.9 Hz, 3H), 1.20 (d, J=6.9 Hz, 3H). Example 322: slower eluting isomer; 39% yield; LCMS: m/z=544.2 [M+H]⁺; ¹H NMR (methanol-d₄): δ=8.10 (d, J=8.7 Hz, 1H), 7.56˜7.46 (m, 2H), 7.38˜7.32 (m, 1H), 7.35 (t, J=72.9 Hz, 1H), 7.25 (dd, J=7.8, 0.9 Hz, 1H), 6.53 (d, J=8.7 Hz, 1H), 4.46˜4.41 (m, 1H), 3.81 (s, 3H), 3.17 (sept, J=6.9 Hz, 1H), 2.36˜2.31 (m, 1H), 2.22˜2.18 (m, 1H), 2.03˜1.83 (m, 3H), 1.63˜1.49 (m, 3H), 1.29 (d, J=6.9 Hz, 3H), 1.18 (d, J=6.9 Hz, 3H).

The following examples were prepared in an analogous fashion to Example 321 and Example 322, but substituting Example 276 with the requisite starting ketone.

| | | | LCMS |
|---|---|---|---|
| Example | Starting Ketone | Structure | (m/z) |
| 323 | 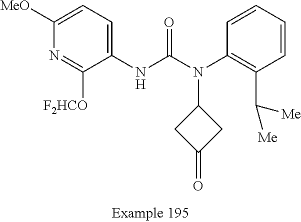 [SMILES: COc1ccc(NC(=O)N(c2ccccc2C(C)C)C2CC(=O)C2)c(OC(F)F)n1] | 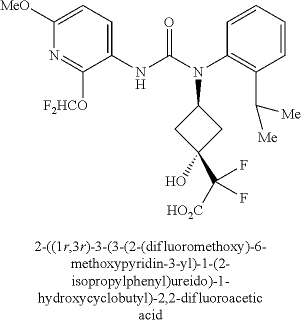 [SMILES: COc1ccc(NC(=O)N(c2ccccc2C(C)C)[C@H]2C[C@@](O)(C(F)(F)C(=O)O)C2)c(OC(F)F)n1] | 516.2 [M + H]⁺ |
| 324 | 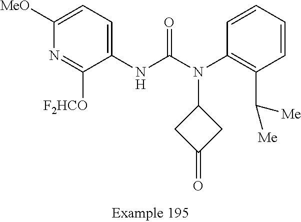 [SMILES: COc1ccc(NC(=O)N(c2ccccc2C(C)C)C2CC(=O)C2)c(OC(F)F)n1] | 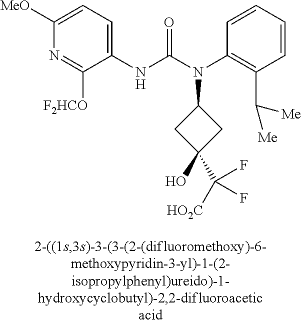 [SMILES: COc1ccc(NC(=O)N(c2ccccc2C(C)C)[C@H]2C[C@](O)(C(F)(F)C(=O)O)C2)c(OC(F)F)n1] | 516.2 [M + H]⁺ |
| 325 | 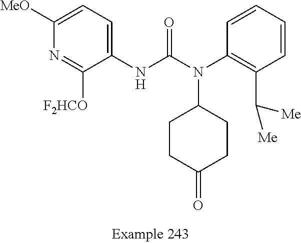 [SMILES: COc1ccc(NC(=O)N(c2ccccc2C(C)C)C2CCC(=O)CC2)c(OC(F)F)n1] | 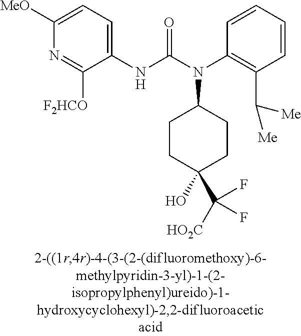 [SMILES: COc1ccc(NC(=O)N(c2ccccc2C(C)C)[C@H]2CC[C@@](O)(C(F)(F)C(=O)O)CC2)c(OC(F)F)n1] | 528.2 [M + H]⁺ |
| 326 | 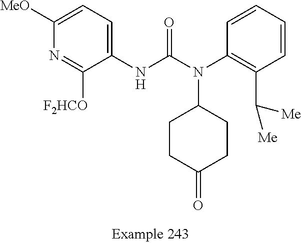 [SMILES: COc1ccc(NC(=O)N(c2ccccc2C(C)C)C2CCC(=O)CC2)c(OC(F)F)n1] | 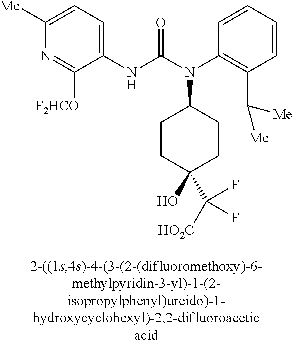 [SMILES: Cc1ccc(NC(=O)N(c2ccccc2C(C)C)[C@H]2CC[C@](O)(C(F)(F)C(=O)O)CC2)c(OC(F)F)n1] | 528.2 [M + H]⁺ |

### 2. Biological Evaluations

In vitro functional assay of lysophosphatidic acid receptor 1 activity

Primary compound plates were prepared in 100% DMSO (Sigma-Aldrich), secondary compound plates were prepared at 10× concentration in DMEM (Invitrogen), and tertiary compound plates were prepared at 3× concentration in assay buffer containing HBSS (no Ca⁺²/Mg⁺², Invitrogen) and 0.1% BSA (Sigma-Aldrich). Fluo-4 NW calcium assay dye (Invitrogen) was prepared as per manufacturer's recommendations in assay buffer. B103 cells stably expressing human LPAR1 (J. Chun lab, UCSD) were grown to confluency in DMEM media (Invitrogen) containing 10% FBS (ATCC), 10% Penicillin-Streptomycin (Sigma-Aldrich) and 50 μg Geneticin (Sigma-Aldrich), and detached with Accutase (Sigma-Aldrich) prior to assay. The freshly detached cells were resuspended in growth media and plated in black, clear-bottom 96-well plates (Costar) containing compound (i.e., secondary compound plate) at a density of 5×10⁴ cells/well. Once plated, cells were left at room temperature for 30 minutes and then transferred to a 37° C., 5% CO₂ incubator for 24 hours. After 24 hours, the growth media was then removed and freshly prepared Fluo-4 NW calcium assay dye was added to cells. Compounds (i.e., tertiary compound plates) were then added back to the dye/cells, returned to the incubator for 30 minutes, and then kept at room temperature for an additional 30 minutes. Finally, lysophosphatidic acid (18:1) at the EC₈₀ concentration was added and calcium flux measured using FlexStation 3 (Molecular Devices). Sigmoidal dose-response curves were generated by measuring luminescence over 45 sec and calculating the area under the curve. Dose response curves and IC₅₀ values were generated using Prism (GraphPad). Compounds were tested at a final concentration range of 100 pM to 10 μM in 0.1% o DMSO. Results are shown in Table 1.

TABLE 1

| Exam- | LPAR1 | Exam- | LPAR1 | Exam- | LPAR1 | Exam- | LPAR1 |
|---|---|---|---|---|---|---|---|
| ple | IC₅₀ | ple | IC₅₀ | ple | IC₅₀ | ple | IC₅₀ |
| 1 | C | 2 | C | 3 | C | 4 | B |
| 5 | B | 6 | B | 7 | C | 8 | C |
| 9 | D | 10 | — | 11 | D | 12 | D |
| 13 | D | 14 | B | 15 | B | 16 | B |
| 17 | B | 18 | B | 19 | C | 20 | B |
| 21 | C | 22 | C | 23 | C | 24 | D |
| 25 | C | 26 | C | 27 | C | 28 | B |
| 29 | B | 30 | C | 31 | C | 32 | C |
| 33 | D | 34 | D | 35 | C | 36 | C |
| 37 | C | 38 | C | 39 | C | 40 | B |
| 41 | C | 42 | C | 43 | C | 44 | B |
| 45 | C | 46 | B | 47 | B | 48 | B |
| 49 | B | 50 | B | 51 | B | 52 | C |
| 53 | C | 54 | B | 55 | C | 56 | C |
| 57 | C | 58 | D | 59 | C | 60 | D |
| 61 | B | 62 | C | 63 | B | 64 | B |
| 65 | B | 66 | A | 67 | B | 68 | B |
| 69 | C | 70 | C | 71 | D | 72 | C |
| 73 | A | 74 | C | 75 | B | 76 | C |
| 77 | B | 78 | B | 79 | A | 80 | C |
| 81 | B | 82 | B | 83 | C | 84 | C |
| 85 | B | 86 | B | 87 | A | 88 | D |
| 89 | C | 90 | B | 91 | B | 92 | B |
| 93 | B | 94 | B | 95 | B | 96 | D |
| 97 | B | 98 | C | 99 | B | 100 | C |
| 101 | D | 102 | B | 103 | D | 104 | C |
| 105 | C | 106 | B | 107 | B | 108 | C |
| 109 | B | 110 | B | 111 | B | 112 | D |
| 113 | B | 114 | B | 115 | B | 116 | C |
| 117 | B | 118 | C | 119 | C | 120 | C |
| 121 | B | 122 | B | 123 | C | 124 | C |
| 125 | B | 126 | B | 127 | B | 128 | B |
| 129 | B | 130 | D | 131 | C | 132 | D |
| 133 | D | 134 | D | 135 | C | 136 | C |
| 137 | D | 138 | B | 139 | D | 140 | C |
| 141 | C | 142 | D | 143 | D | 144 | B |
| 145 | C | 146 | B | 147 | A | 148 | A |
| 149 | B | 150 | A | 151 | B | 152 | C |
| 153 | A | 154 | A | 155 | B | 156 | A |
| 157 | B | 158 | B | 159 | C | 160 | A |
| 161 | B | 162 | C | 163 | D | 164 | B |
| 165 | A | 166 | B | 167 | B | 168 | B |
| 169 | B | 170 | A | 171 | B | 172 | B |
| 173 | B | 174 | C | 175 | A | 176 | B |
| 177 | B | 178 | B | 179 | D | 180 | D |
| 181 | B | 182 | C | 183 | B | 184 | C |
| 185 | C | 186 | D | 187 | D | 188 | C |
| 189 | C | 190 | C | 191 | B | 192 | B |
| 193 | C | 194 | C | 195 | B | 196 | D |
| 197 | D | 198 | C | 199 | A | 200 | B |
| 201 | B | 202 | B | 203 | B | 204 | A |
| 205 | A | 206 | B | 207 | A | 208 | B |
| 209 | B | 210 | A | 211 | A | 212 | A |
| 213 | B | 214 | B | 215 | B | 216 | B |
| 217 | B | 218 | B | 219 | — | 220 | — |
| 221 | A | 222 | B | 223 | B | 224 | A |
| 225 | B | 226 | C | 227 | B | 228 | C |
| 229 | B | 230 | C | 231 | — | 232 | B |
| 233 | B | 234 | B | 235 | B | 236 | C |
| 237 | C | 238 | C | 239 | C | 240 | C |
| 241 | C | 242 | C | 243 | C | 244 | D |
| 245 | D | 246 | D | 247 | B | 248 | C |
| 249 | B | 250 | C | 251 | D | 252 | C |
| 253 | B | 254 | D | 255 | B | 256 | C |
| 257 | A | 258 | C | 259 | B | 260 | D |
| 261 | C | 262 | A | 263 | — | 264 | C |
| 265 | D | 266 | C | 267 | B | 268 | C |
| 269 | B | 270 | — | 271 | C | 272 | B |
| 273 | B | 274 | C | 275 | B | 276 | C |
| 277 | — | 278 | B | 279 | A | 280 | A |
| 281 | B | 282 | C | 283 | B | 284 | A |
| 285 | A | 286 | B | 287 | C | 288 | B |
| 289 | A | 290 | B | 291 | A | 292 | A |
| 293 | B | 294 | C | 295 | C | 296 | C |
| 297 | B | 298 | A | 299 | A | 300 | C |
| 301 | B | 302 | B | 303 | A | 304 | A |
| 305 | B | 306 | C | 307 | C | 308 | C |
| 309 | A | 310 | B | 311 | C | 312 | C |
| 313 | C | 314 | C | 315 | B | 316 | B |
| 317 | C | 318 | C | 319 | — | 320 | B |
| 321 | A | 322 | A | 323 | A | 324 | A |
| 325 | B | 326 | A | 327 | A | 328 | B |
| 329 | A | 330 | A | 331 | A | 332 | B |
| 333 | A | 334 | A | 335 | B | 336 | B |
| 337 | B | 338 | B | 339 | B | 340 | B |

A = IC₅₀ of less than 10 nM;
B = IC₅₀ of less than 100 nM but greater or equal to 10 nM;
C = IC₅₀ of less than 1,000 nM but greater or equal to 100 nM;
D = IC₅₀ of greater or equal to 1,000 nM.

1. A compound, or a pharmaceutically acceptable salt or solvate thereof, having the formula:
wherein 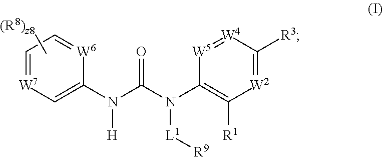
L¹ is a bond or substituted or unsubstituted C₁-C₅ alkylene;
R¹ is unsubstituted C₂-C₅ alkyl;
W² is N or C(R²);
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶);
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R⁹ is substituted or unsubstituted cycloalkyl or substituted or unsubstituted heterocycloalkyl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, and R^{8D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, and X⁸ are independently —F, —Cl, —Br, or —I;
n2, n3, n4, n5, n6, n7, and n8 are independently an integer from 0 to 4;
m2, m3, m4, m5, m6, m7, m8, v2, v3, v4, v5, v6, v7, and v8 are independently 1 or 2; and
z8 is an integer from 0 to 3.

2. The compound of claim 1, having the formula:

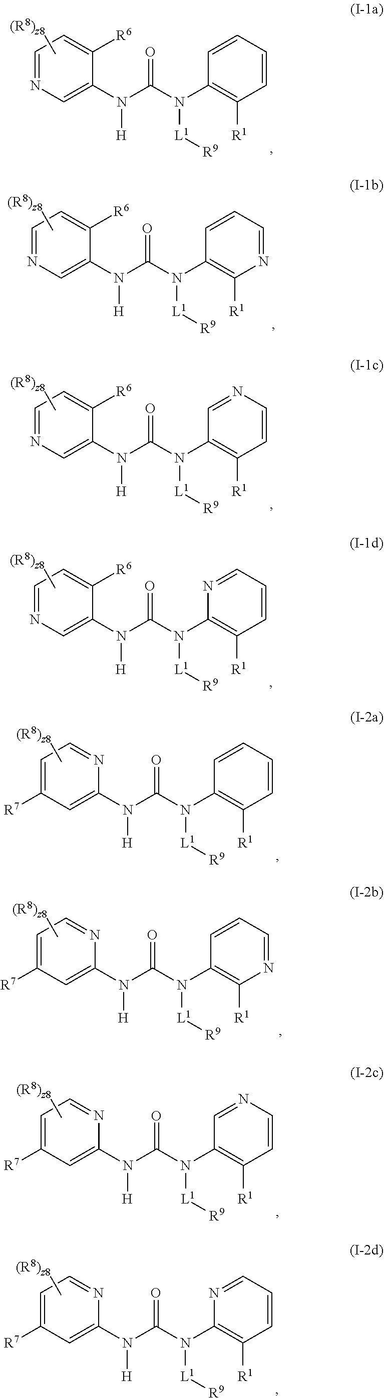

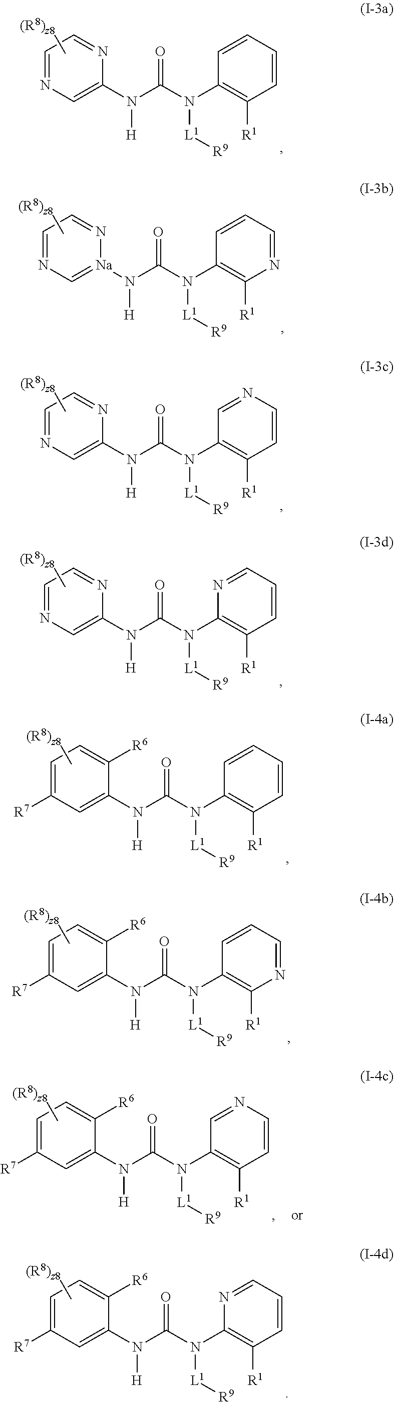

3.-6. (canceled)

7. The compound of claim 1, wherein R¹ is isopropyl.

8. A compound, or a pharmaceutically acceptable salt or solvate thereof, having the formula: 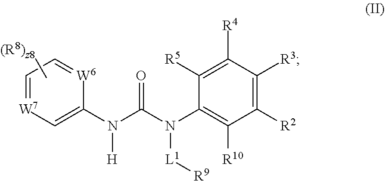
wherein
L¹ is a bond or substituted or unsubstituted C₁-C₅ alkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶);
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R⁹ is substituted or unsubstituted cycloalkyl or substituted or unsubstituted heterocycloalkyl;
R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, and X¹⁰ are independently —F, —Cl, —Br, or —I;
n2, n3, n4, n5, n6, n7, n8, and n10 are independently an integer from 0 to 4;
m2, m3, m4, m5, m6, m7, m8, m10, v2, v3, v4, v5, v6, v7, v8, and v10 are independently 1 or 2; and
z8 is an integer from 0 to 3;
wherein at least one of W⁶ or W⁷ is N;
wherein if W⁶ is C(R⁶) or W⁷ is C(R⁷), then R¹⁰ is not hydrogen;
wherein if W⁶ and W⁷ are both N, then R³ is not —S(O)₂CH₃; and
wherein if W⁶ is CH and W⁷ is N, then -L¹-R⁹ is not

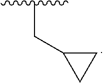 [SMILES: *CC1CC1]

9. The compound of claim 8, having the formula:

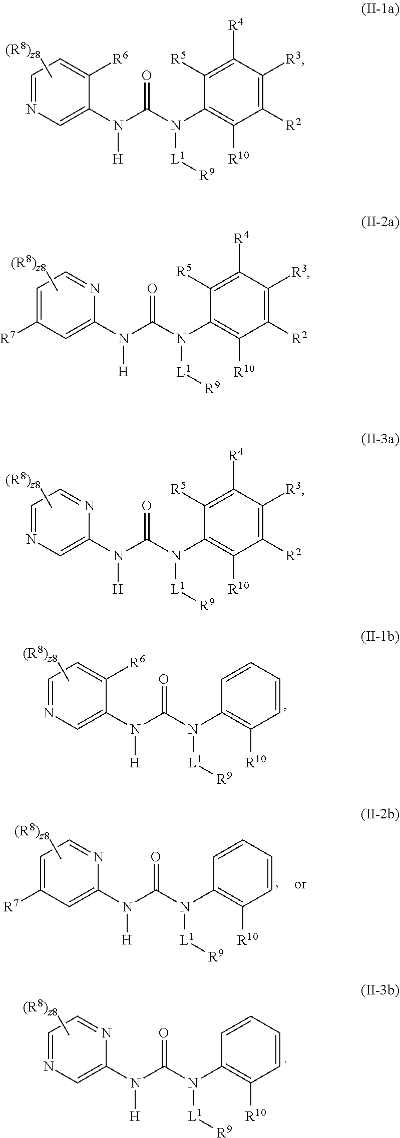

10. (canceled)

11. (canceled)

12. The compound of claim 8, wherein R¹⁰ is isopropyl.

13. (canceled)

14. The compound of claim 1, wherein R⁶ is hydrogen, —OCHF₂, unsubstituted methoxy, or unsubstituted isopropoxy.

15. (canceled)

16. (canceled)

17. The compound of claim 1, wherein R⁸ is independently halogen, —CF₃, —CHF₂, —CN, —OCHF₂, —C(O)R^{8C}, —C(O)OR^{8C}, —OR^{8D}, unsubstituted C₁-C₆ alkyl, unsubstituted 2 to 8 membered heteroalkyl, unsubstituted C₃-C₈ cycloalkyl, or unsubstituted phenyl.

18.-22. (canceled)

23. The compound of claim 1, wherein L¹ is a bond or unsubstituted methylene.

24. (canceled)

25. The compound of claim 1, wherein R⁹ is an R¹¹-substituted or unsubstituted cycloalkyl or R¹¹-substituted or unsubstituted heterocycloalkyl;
R¹¹ is independently oxo, halogen, —CX¹¹₃, —CHX¹¹₂, —CH₂X¹¹, —OCX¹¹₃, —OCH₂X¹¹, —OCHX¹¹₂, —CN, —SO₁₁R^{11D}, —SOᵥ₁₁NR^{11A}R^{11B}, —NR^{11C}N^{11A}R^{11B}, —ONR^{11A}R^{11B}, —NHC(O)NR^{11C}NR^{11A}R^{11B}, —NHC(O)NR^{11A}R^{11B}, —N(O)ₘ₁, —NR^{11A}R^{11B}, —C(O)R^{11C}, —C(O)OR^{11C}, —C(O)NR^{11A}R^{11B}, —OR^{11D}, —SR^{11D}, —NR^{11A}SO₂R^{11D}, —NR^{11A}C(O)R^{11C}, —NR^{11A}C(O)OR^{11C}, —NR^{11A}OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{11A}, R^{11B}, R^{11C}, and R^{11D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl;
X¹¹ is independently —F, —Cl, —Br, or —I;
n11 is independently an integer from 0 to 4; and
m11 and v11 are independently 1 or 2.

26. (canceled)

27. (canceled)

28. The compound of claim 25, wherein R⁹ is 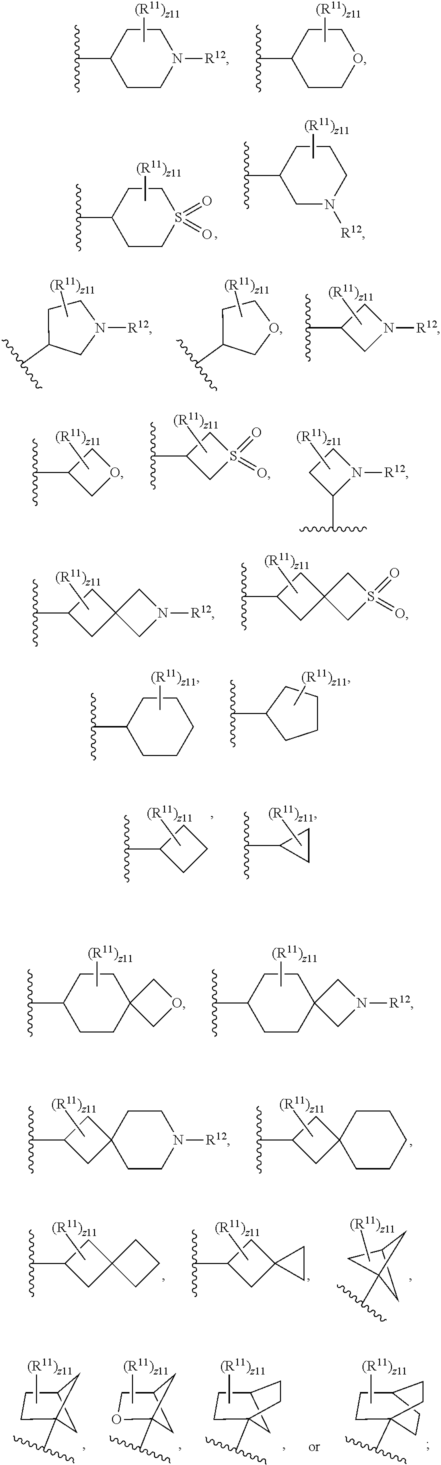
R¹² is hydrogen, halogen, —CX¹²₃, —CHX¹²₂, —CH₂X¹², —OCX¹²₃, —OCH₂X¹², —OCHX¹²₂, —SOₙ₁₂R^{12D}, —SOᵥ₁₂NR^{12A}R^{12B}, —C(O)R^{12C}, —C(O)OR^{12C}, —C(O)NR^{12A}R^{12B}, —OR^{12D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{12A}, R^{12B}, R^{12C}, and R^{12D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl;
X¹² is independently —F, —Cl, —Br, or —I;
n12 is independently an integer from 0 to 4;
v12 is independently 1 or 2; and
z11 is an integer from 0 to 13.

29.-38. (canceled)

39. The compound of claim 1, wherein R⁹ is

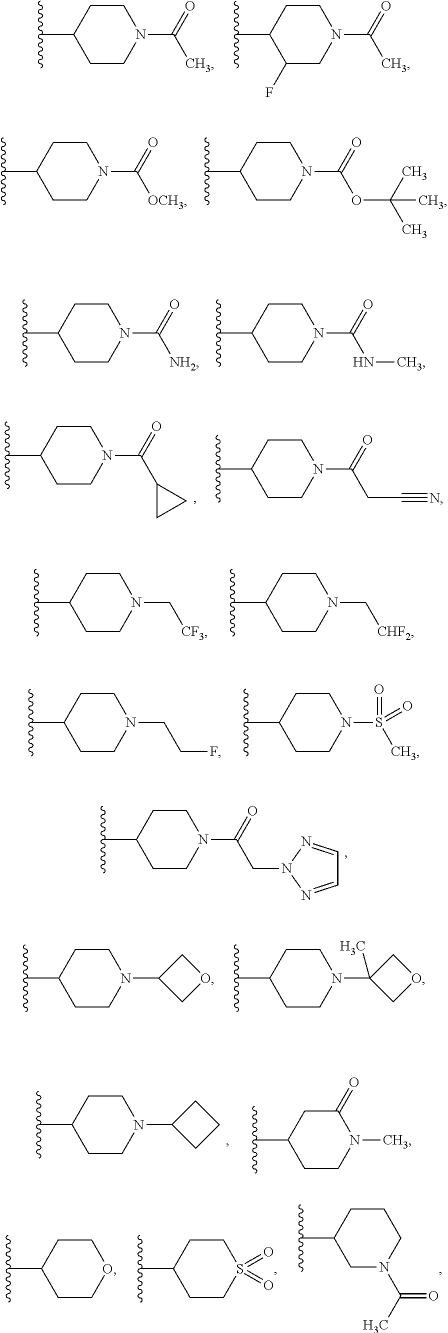

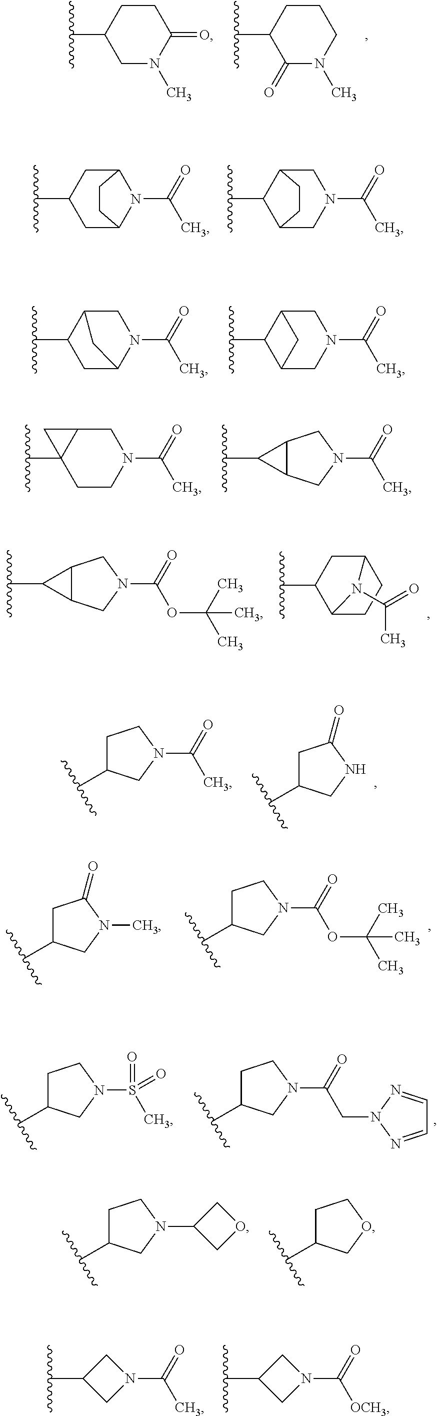

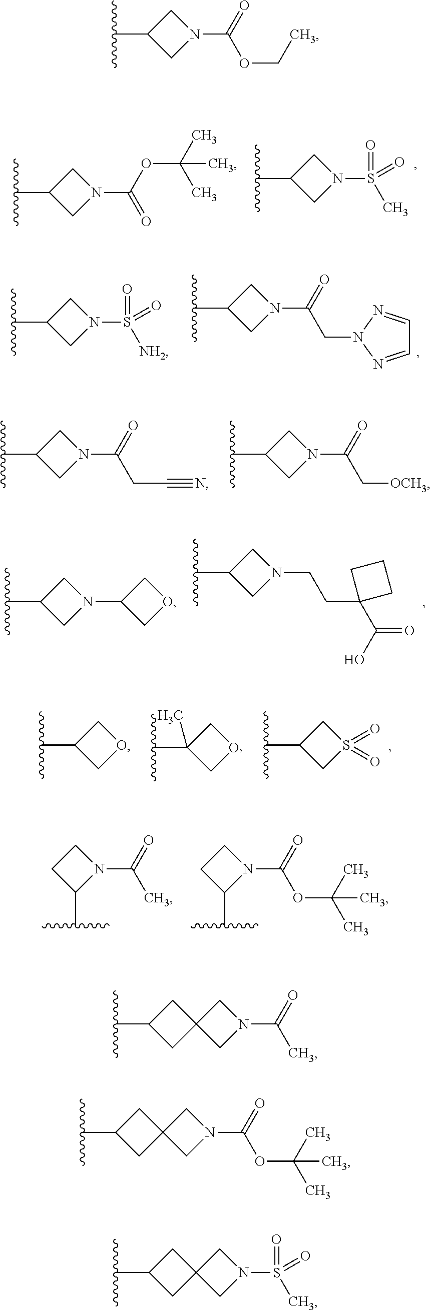

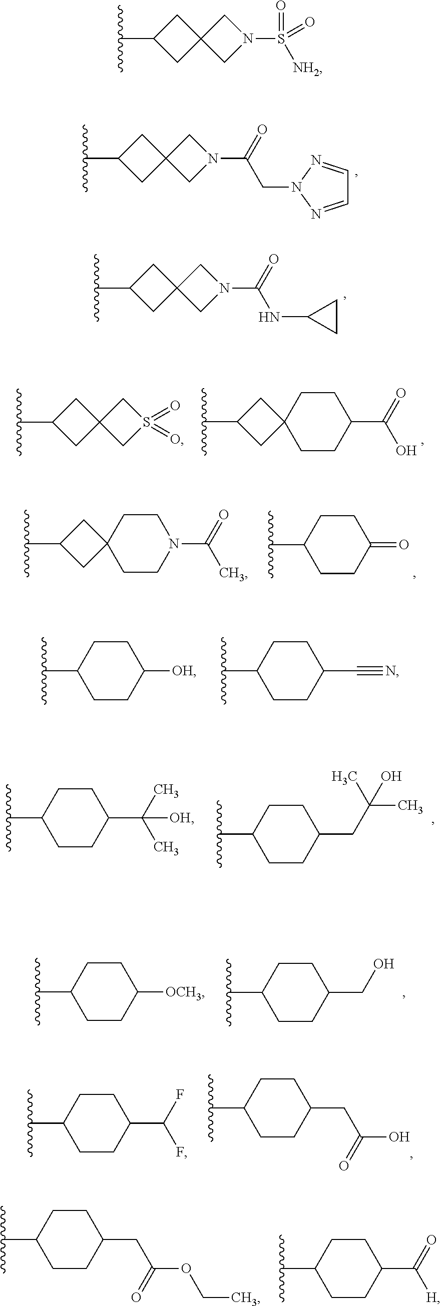

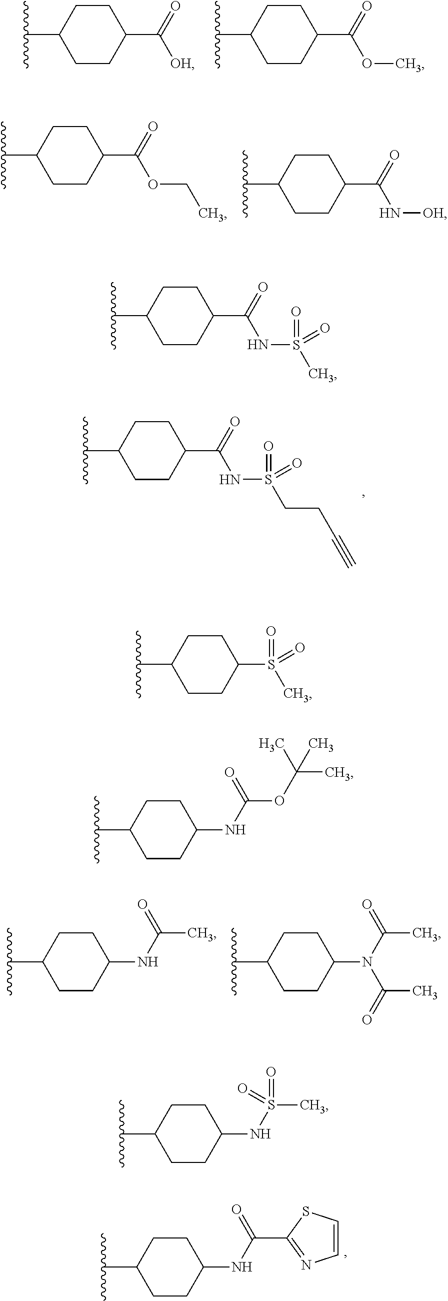

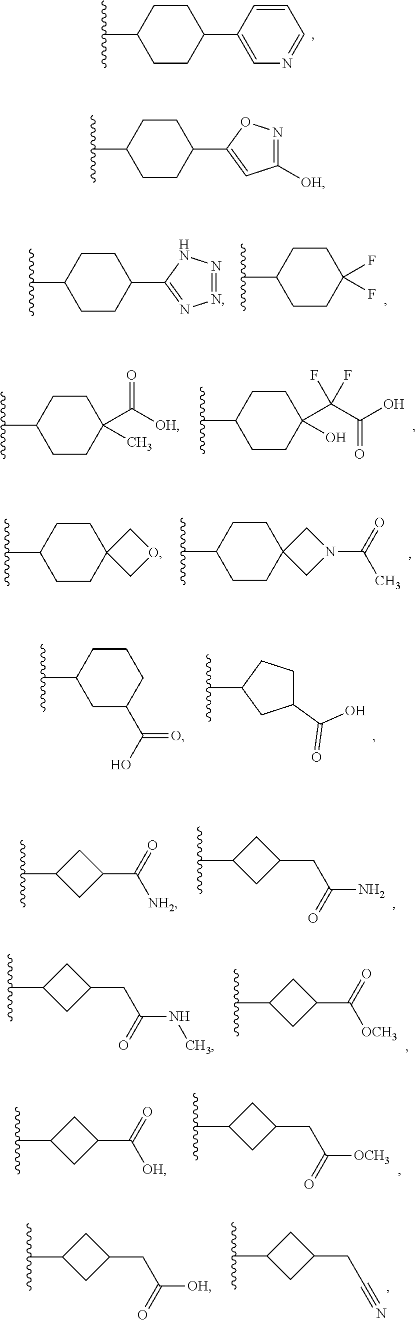

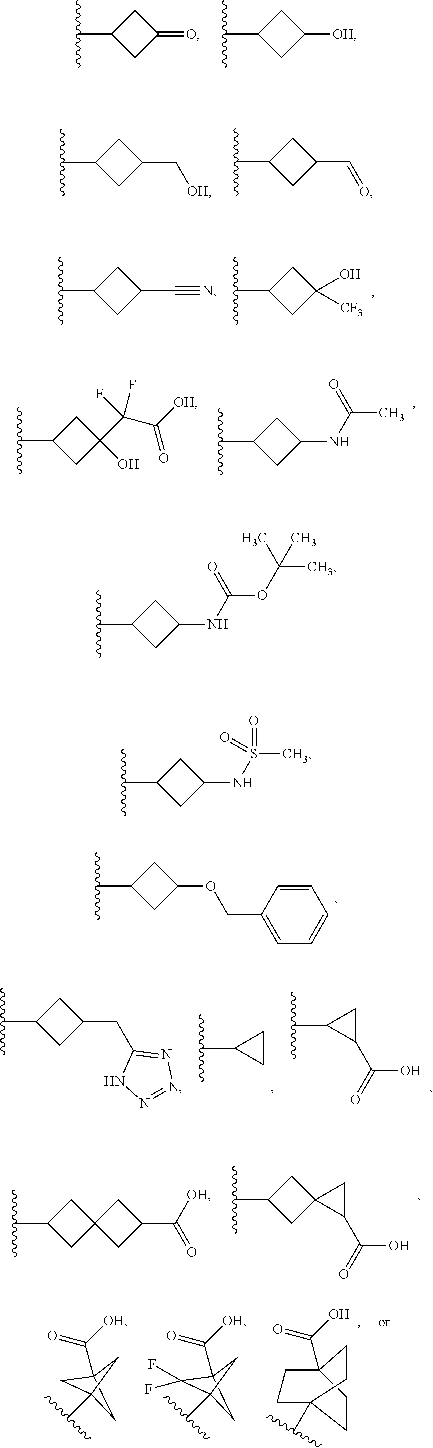

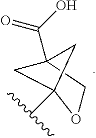 [SMILES: *C12CC(C(=O)O)(CO1)C2]

40. A compound, or a pharmaceutically acceptable salt or solvate thereof, having the formula:

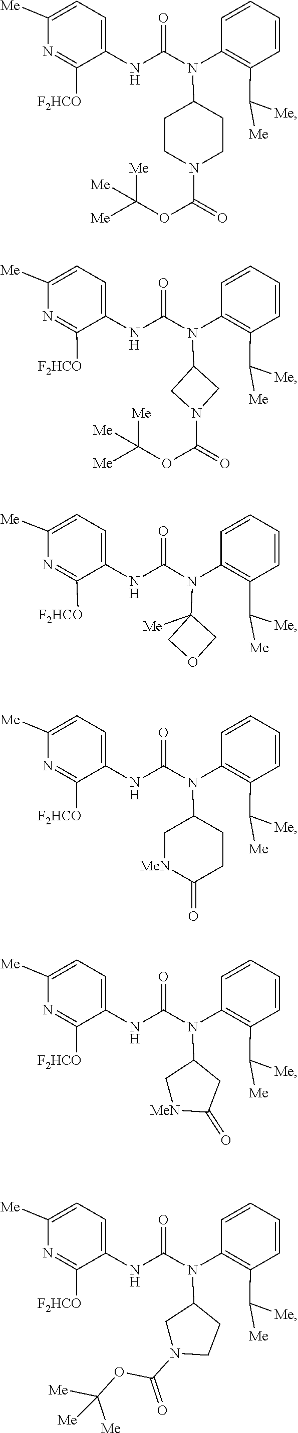 [SMILES: Cc1ccc(NC(=O)N(c2ccccc2C(C)C)C2CCN(C(=O)OC(C)(C)C)CC2)c(OC(F)F)n1 ; Cc1ccc(NC(=O)N(c2ccccc2C(C)C)C2CN(C(=O)OC(C)(C)C)C2)c(OC(F)F)n1 ; Cc1ccc(NC(=O)N(c2ccccc2C(C)C)C2(C)COC2)c(OC(F)F)n1 ; Cc1ccc(NC(=O)N(c2ccccc2C(C)C)C2CCC(=O)N(C)C2)c(OC(F)F)n1 ; Cc1ccc(NC(=O)N(c2ccccc2C(C)C)C2CC(=O)N(C)C2)c(OC(F)F)n1 ; Cc1ccc(NC(=O)N(c2ccccc2C(C)C)C2CCN(C(=O)OC(C)(C)C)C2)c(OC(F)F)n1]

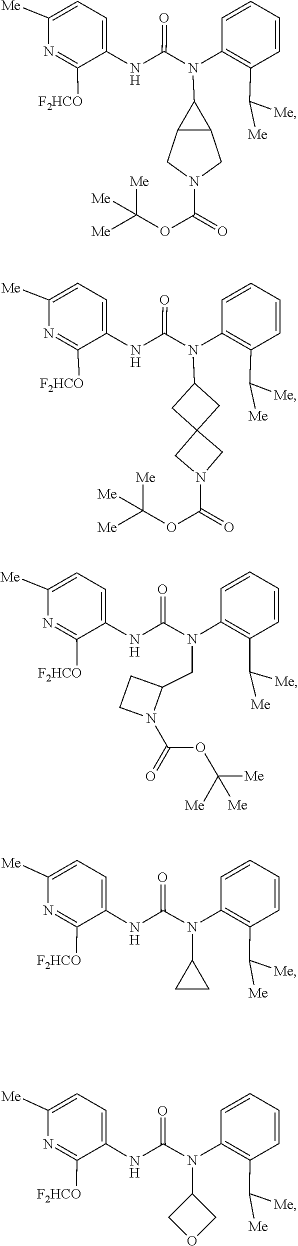 [SMILES: Cc1ccc(NC(=O)N(c2ccccc2C(C)C)C2C3CN(C(=O)OC(C)(C)C)CC32)c(OC(F)F)n1 ; Cc1ccc(NC(=O)N(c2ccccc2C(C)C)C2CC3(C2)CN(C(=O)OC(C)(C)C)C3)c(OC(F)F)n1 ; Cc1ccc(NC(=O)N(CC2CCN2C(=O)OC(C)(C)C)c2ccccc2C(C)C)c(OC(F)F)n1 ; Cc1ccc(NC(=O)N(c2ccccc2C(C)C)C2CC2)c(OC(F)F)n1 ; Cc1ccc(NC(=O)N(c2ccccc2C(C)C)C2COC2)c(OC(F)F)n1]

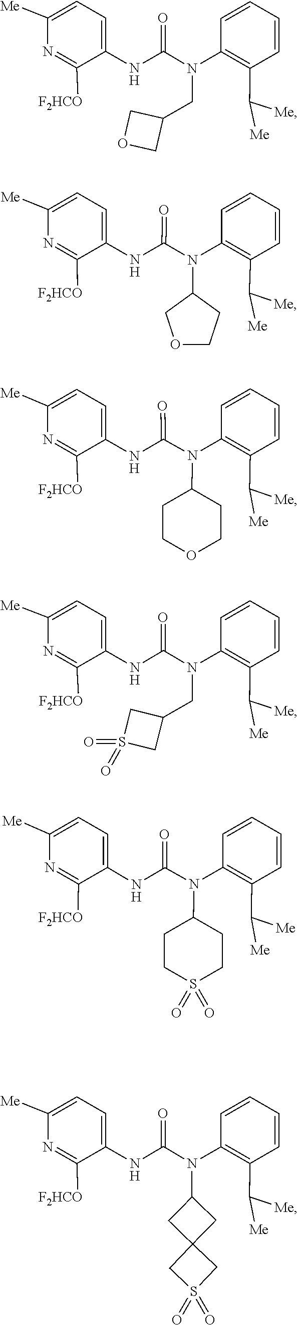 [SMILES: Cc1ccc(NC(=O)N(CC2COC2)c2ccccc2C(C)C)c(OC(F)F)n1 ; Cc1ccc(NC(=O)N(c2ccccc2C(C)C)C2CCOC2)c(OC(F)F)n1 ; Cc1ccc(NC(=O)N(c2ccccc2C(C)C)C2CCOCC2)c(OC(F)F)n1 ; Cc1ccc(NC(=O)N(CC2CS(=O)(=O)C2)c2ccccc2C(C)C)c(OC(F)F)n1 ; Cc1ccc(NC(=O)N(c2ccccc2C(C)C)C2CCS(=O)(=O)CC2)c(OC(F)F)n1 ; Cc1ccc(NC(=O)N(c2ccccc2C(C)C)C2CC3(C2)CS(=O)(=O)C3)c(OC(F)F)n1]

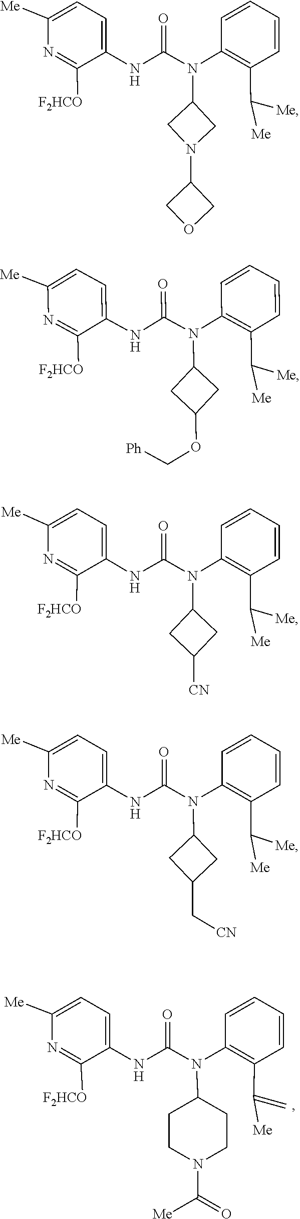 [SMILES: Cc1ccc(NC(=O)N(c2ccccc2C(C)C)C2CN(C3COC3)C2)c(OC(F)F)n1 ; Cc1ccc(NC(=O)N(c2ccccc2C(C)C)C2CC(OCc3ccccc3)C2)c(OC(F)F)n1 ; Cc1ccc(NC(=O)N(c2ccccc2C(C)C)C2CC(C#N)C2)c(OC(F)F)n1 ; Cc1ccc(NC(=O)N(c2ccccc2C(C)C)C2CC(CC#N)C2)c(OC(F)F)n1 ; C=C(C)c1ccccc1N(C(=O)Nc1ccc(C)nc1OC(F)F)C1CCN(C(C)=O)CC1]

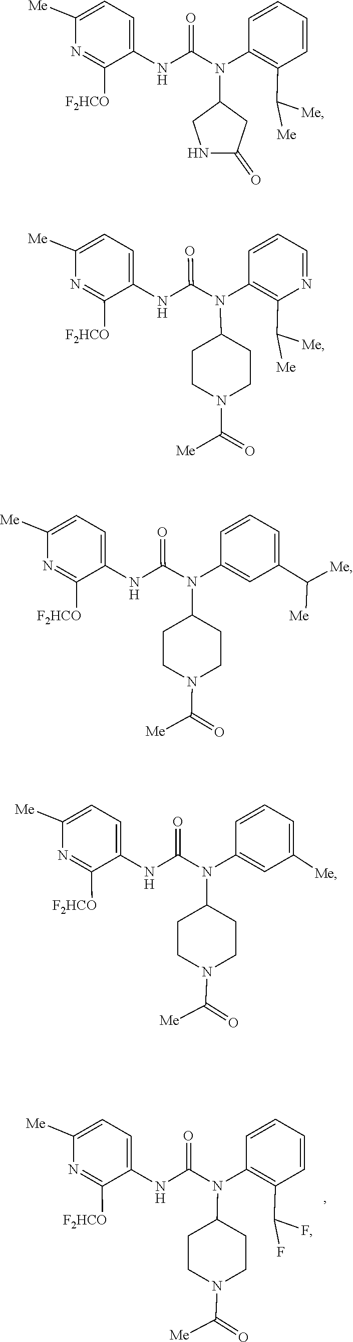 [SMILES: Cc1ccc(NC(=O)N(c2ccccc2C(C)C)C2CNC(=O)C2)c(OC(F)F)n1 ; CC(=O)N1CCC(N(C(=O)Nc2ccc(C)nc2OC(F)F)c2cccnc2C(C)C)CC1 ; CC(=O)N1CCC(N(C(=O)Nc2ccc(C)nc2OC(F)F)c2cccc(C(C)C)c2)CC1 ; CC(=O)N1CCC(N(C(=O)Nc2ccc(C)nc2OC(F)F)c2cccc(C)c2)CC1 ; CC(=O)N1CCC(N(C(=O)Nc2ccc(C)nc2OC(F)F)c2ccccc2C(F)F)CC1]

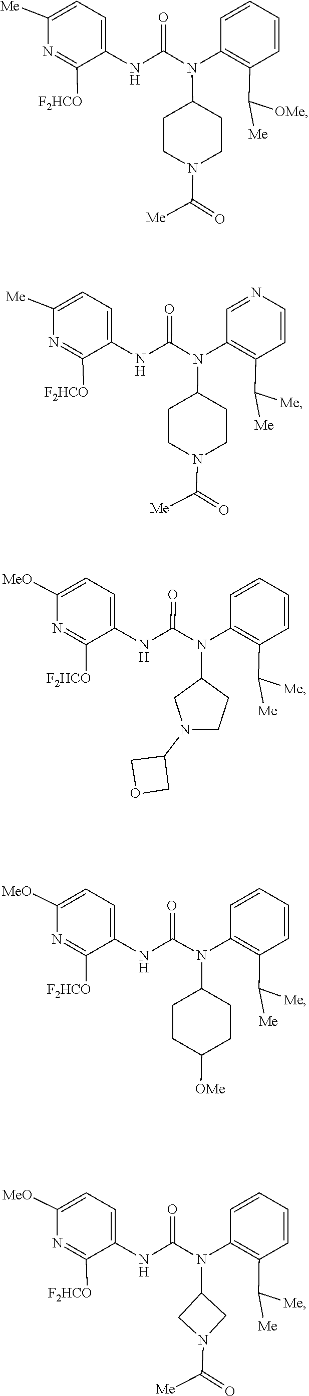 [SMILES: COC(C)c1ccccc1N(C(=O)Nc1ccc(C)nc1OC(F)F)C1CCN(C(C)=O)CC1 ; CC(=O)N1CCC(N(C(=O)Nc2ccc(C)nc2OC(F)F)c2cnccc2C(C)C)CC1 ; COc1ccc(NC(=O)N(c2ccccc2C(C)C)C2CCN(C3COC3)C2)c(OC(F)F)n1 ; COc1ccc(NC(=O)N(c2ccccc2C(C)C)C2CCC(OC)CC2)c(OC(F)F)n1 ; COc1ccc(NC(=O)N(c2ccccc2C(C)C)C2CN(C(C)=O)C2)c(OC(F)F)n1]

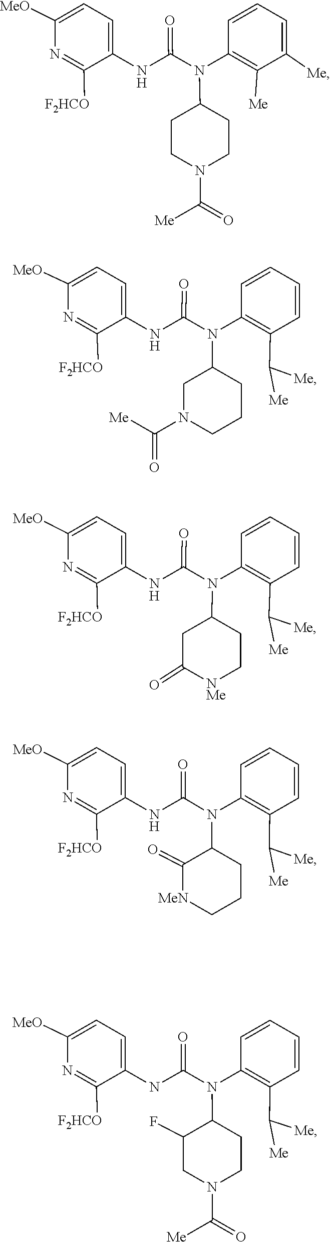

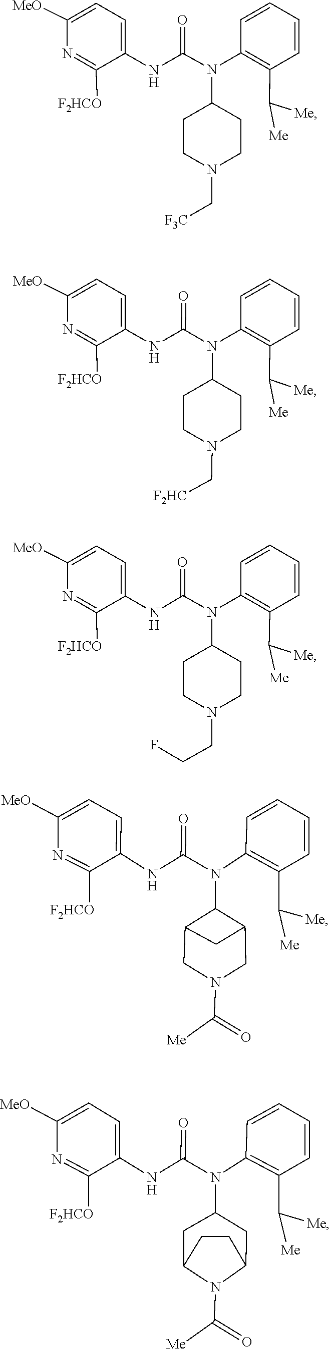 [SMILES: COc1ccc(NC(=O)N(c2ccccc2C(C)C)C2CCN(CC(F)(F)F)CC2)c(OC(F)F)n1 ; COc1ccc(NC(=O)N(c2ccccc2C(C)C)C2CCN(CC(F)F)CC2)c(OC(F)F)n1 ; COc1ccc(NC(=O)N(c2ccccc2C(C)C)C2CCN(CCF)CC2)c(OC(F)F)n1 ; COc1ccc(NC(=O)N(c2ccccc2C(C)C)C2C3CC2CN(C(C)=O)C3)c(OC(F)F)n1 ; COc1ccc(NC(=O)N(c2ccccc2C(C)C)C2CC3CCC(C2)N3C(C)=O)c(OC(F)F)n1]

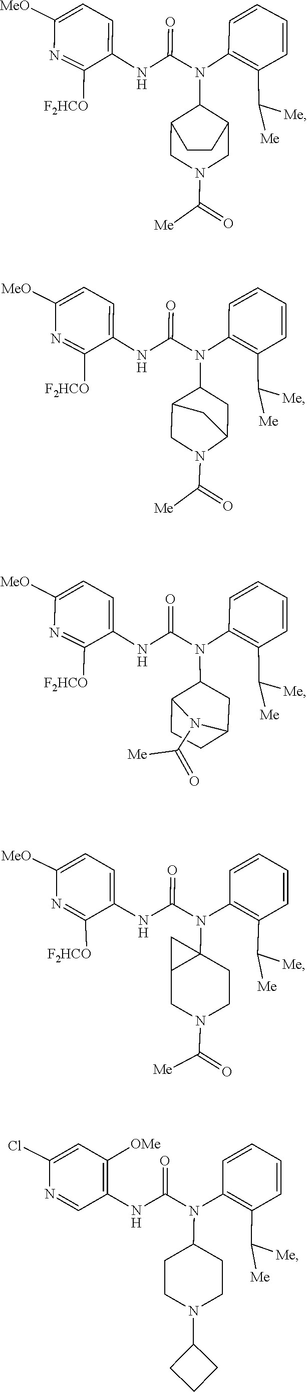 [SMILES: COc1ccc(NC(=O)N(c2ccccc2C(C)C)C2C3CCC2CN(C(C)=O)C3)c(OC(F)F)n1 ; COc1ccc(NC(=O)N(c2ccccc2C(C)C)C2CC3CC2CN3C(C)=O)c(OC(F)F)n1 ; COc1ccc(NC(=O)N(c2ccccc2C(C)C)C2CC3CCC2N3C(C)=O)c(OC(F)F)n1 ; COc1ccc(NC(=O)N(c2ccccc2C(C)C)C23CCN(C(C)=O)CC2C3)c(OC(F)F)n1 ; COc1cc(Cl)ncc1NC(=O)N(c1ccccc1C(C)C)C1CCN(C2CCC2)CC1]

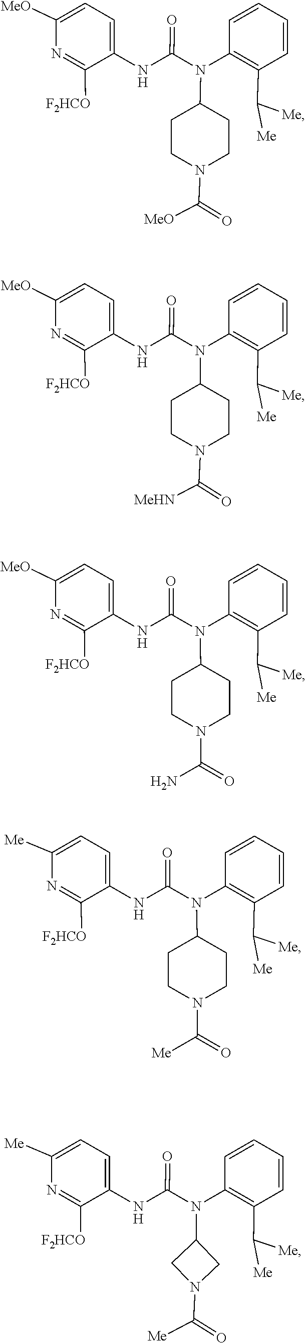 [SMILES: COC(=O)N1CCC(N(C(=O)Nc2ccc(OC)nc2OC(F)F)c2ccccc2C(C)C)CC1 ; CNC(=O)N1CCC(N(C(=O)Nc2ccc(OC)nc2OC(F)F)c2ccccc2C(C)C)CC1 ; COc1ccc(NC(=O)N(c2ccccc2C(C)C)C2CCN(C(N)=O)CC2)c(OC(F)F)n1 ; CC(=O)N1CCC(N(C(=O)Nc2ccc(C)nc2OC(F)F)c2ccccc2C(C)C)CC1 ; CC(=O)N1CC(N(C(=O)Nc2ccc(C)nc2OC(F)F)c2ccccc2C(C)C)C1]

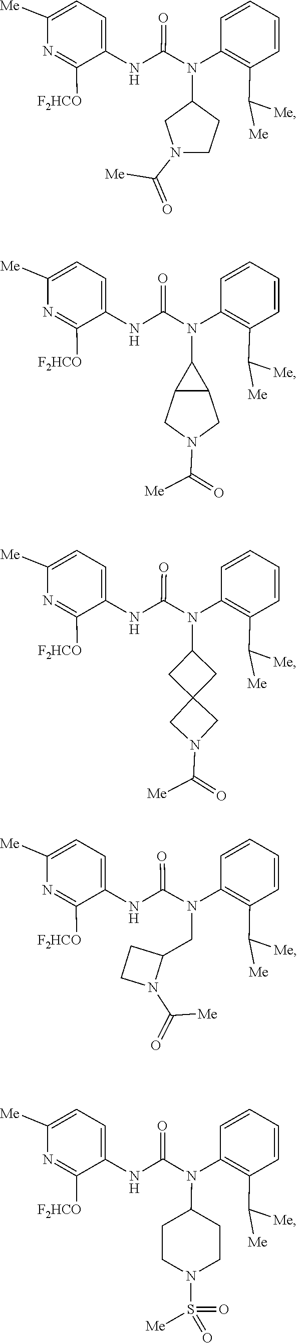 [SMILES: CC(=O)N1CCC(N(C(=O)Nc2ccc(C)nc2OC(F)F)c2ccccc2C(C)C)C1 ; CC(=O)N1CC2C(C1)C2N(C(=O)Nc1ccc(C)nc1OC(F)F)c1ccccc1C(C)C ; CC(=O)N1CC2(CC(N(C(=O)Nc3ccc(C)nc3OC(F)F)c3ccccc3C(C)C)C2)C1 ; CC(=O)N1CCC1CN(C(=O)Nc1ccc(C)nc1OC(F)F)c1ccccc1C(C)C ; Cc1ccc(NC(=O)N(c2ccccc2C(C)C)C2CCN(S(C)(=O)=O)CC2)c(OC(F)F)n1]

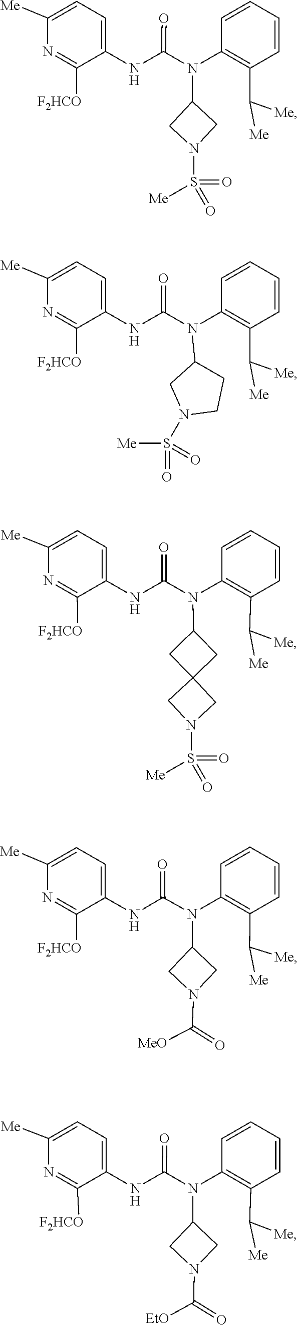 [SMILES: Cc1ccc(NC(=O)N(c2ccccc2C(C)C)C2CN(S(C)(=O)=O)C2)c(OC(F)F)n1 ; Cc1ccc(NC(=O)N(c2ccccc2C(C)C)C2CCN(S(C)(=O)=O)C2)c(OC(F)F)n1 ; Cc1ccc(NC(=O)N(c2ccccc2C(C)C)C2CC3(C2)CN(S(C)(=O)=O)C3)c(OC(F)F)n1 ; COC(=O)N1CC(N(C(=O)Nc2ccc(C)nc2OC(F)F)c2ccccc2C(C)C)C1 ; CCOC(=O)N1CC(N(C(=O)Nc2ccc(C)nc2OC(F)F)c2ccccc2C(C)C)C1]

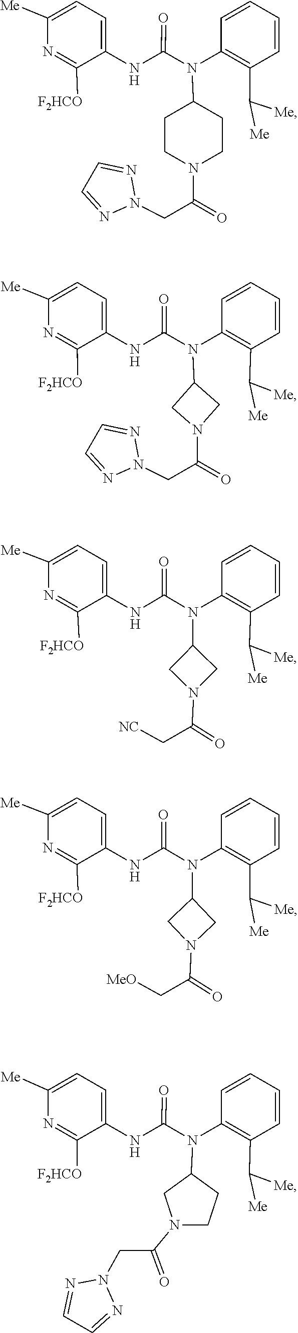 [SMILES: Cc1ccc(NC(=O)N(c2ccccc2C(C)C)C2CCN(C(=O)Cn3nccn3)CC2)c(OC(F)F)n1 ; Cc1ccc(NC(=O)N(c2ccccc2C(C)C)C2CN(C(=O)Cn3nccn3)C2)c(OC(F)F)n1 ; Cc1ccc(NC(=O)N(c2ccccc2C(C)C)C2CN(C(=O)CC#N)C2)c(OC(F)F)n1 ; COCC(=O)N1CC(N(C(=O)Nc2ccc(C)nc2OC(F)F)c2ccccc2C(C)C)C1 ; Cc1ccc(NC(=O)N(c2ccccc2C(C)C)C2CCN(C(=O)Cn3nccn3)C2)c(OC(F)F)n1]

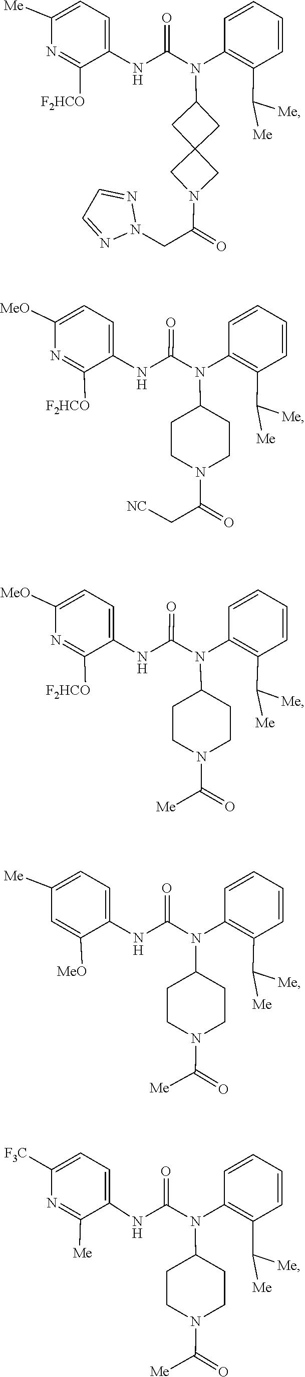 [SMILES: Cc1ccc(NC(=O)N(c2ccccc2C(C)C)C2CC3(C2)CN(C(=O)Cn2nccn2)C3)c(OC(F)F)n1 ; COc1ccc(NC(=O)N(c2ccccc2C(C)C)C2CCN(C(=O)CC#N)CC2)c(OC(F)F)n1 ; COc1ccc(NC(=O)N(c2ccccc2C(C)C)C2CCN(C(C)=O)CC2)c(OC(F)F)n1 ; COc1cc(C)ccc1NC(=O)N(c1ccccc1C(C)C)C1CCN(C(C)=O)CC1 ; CC(=O)N1CCC(N(C(=O)Nc2ccc(C(F)(F)F)nc2C)c2ccccc2C(C)C)CC1]

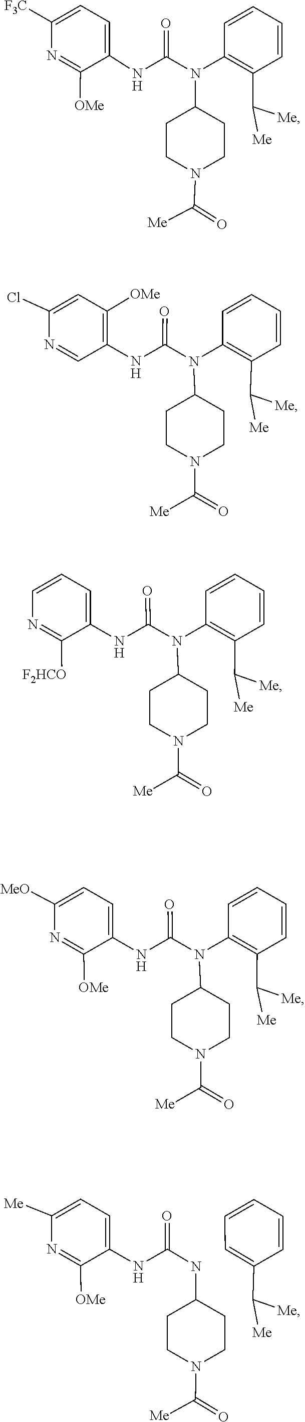

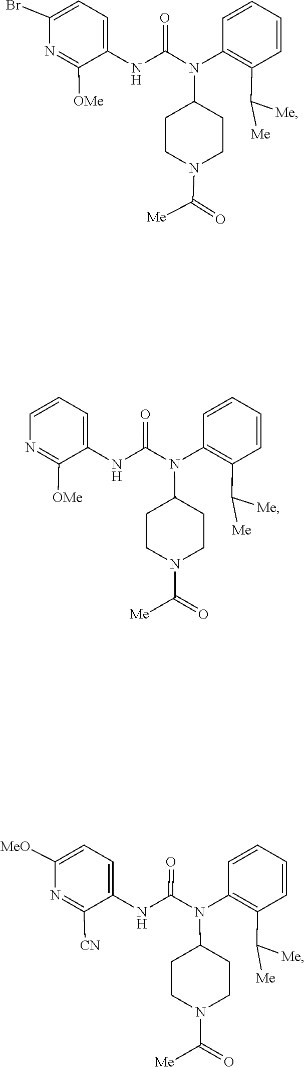 [SMILES: COc1nc(Br)ccc1NC(=O)N(c1ccccc1C(C)C)C1CCN(C(C)=O)CC1 ; COc1ncccc1NC(=O)N(c1ccccc1C(C)C)C1CCN(C(C)=O)CC1 ; COc1ccc(NC(=O)N(c2ccccc2C(C)C)C2CCN(C(C)=O)CC2)c(C#N)n1]

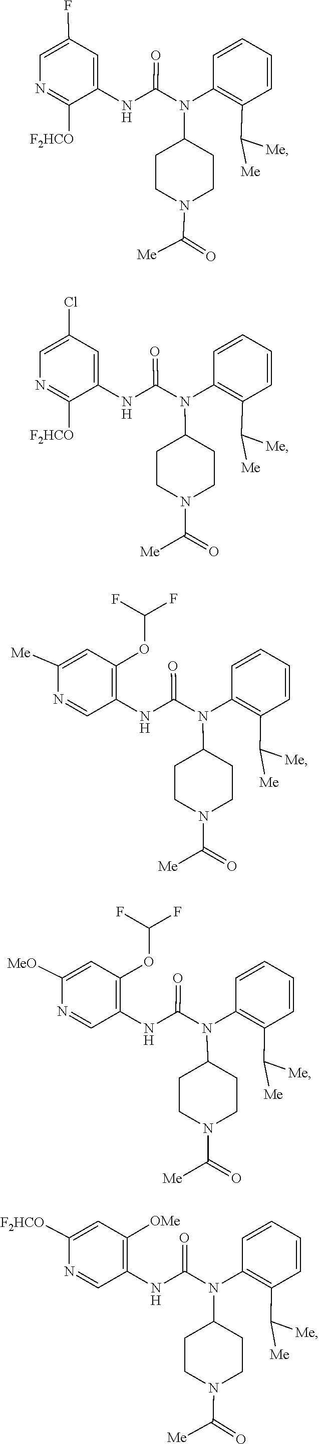

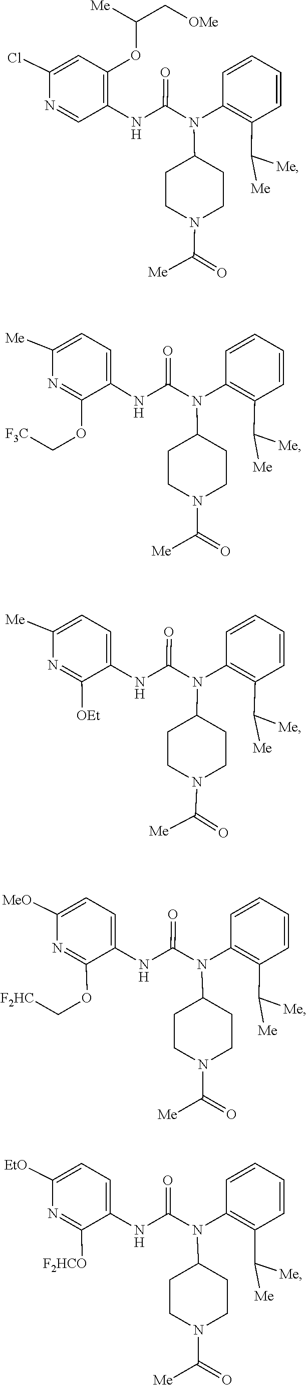 [SMILES: COCC(C)Oc1cc(Cl)ncc1NC(=O)N(c1ccccc1C(C)C)C1CCN(C(C)=O)CC1 ; CC(=O)N1CCC(N(C(=O)Nc2ccc(C)nc2OCC(F)(F)F)c2ccccc2C(C)C)CC1 ; CCOc1nc(C)ccc1NC(=O)N(c1ccccc1C(C)C)C1CCN(C(C)=O)CC1 ; COc1ccc(NC(=O)N(c2ccccc2C(C)C)C2CCN(C(C)=O)CC2)c(OCC(F)F)n1 ; CCOc1ccc(NC(=O)N(c2ccccc2C(C)C)C2CCN(C(C)=O)CC2)c(OC(F)F)n1]

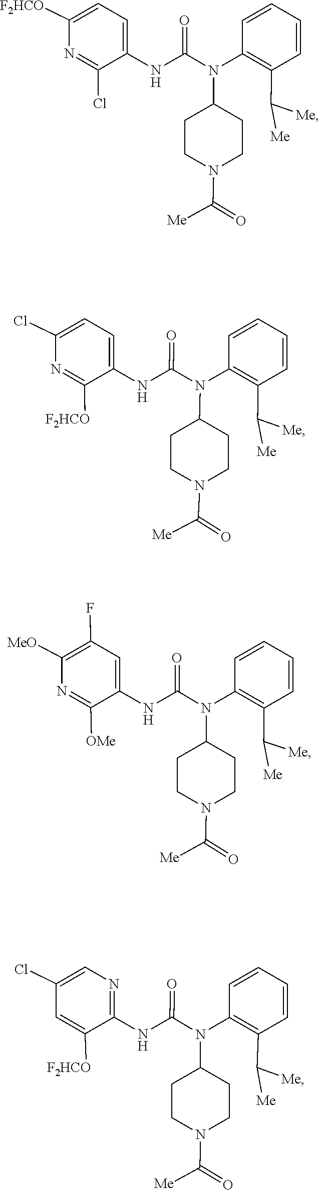 [SMILES: CC(=O)N1CCC(N(C(=O)Nc2ccc(OC(F)F)nc2Cl)c2ccccc2C(C)C)CC1 ; CC(=O)N1CCC(N(C(=O)Nc2ccc(Cl)nc2OC(F)F)c2ccccc2C(C)C)CC1 ; COc1nc(OC)c(NC(=O)N(c2ccccc2C(C)C)C2CCN(C(C)=O)CC2)cc1F ; CC(=O)N1CCC(N(C(=O)Nc2ncc(Cl)cc2OC(F)F)c2ccccc2C(C)C)CC1]

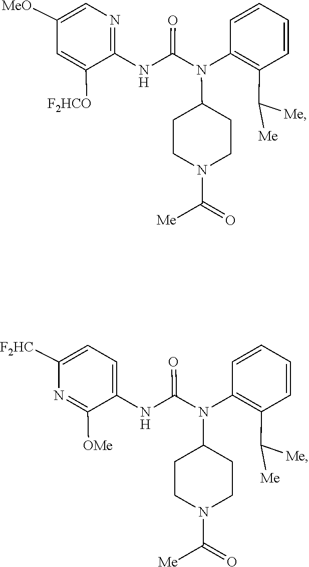 [SMILES: COc1cnc(NC(=O)N(c2ccccc2C(C)C)C2CCN(C(C)=O)CC2)c(OC(F)F)c1 ; COc1nc(C(F)F)ccc1NC(=O)N(c1ccccc1C(C)C)C1CCN(C(C)=O)CC1]

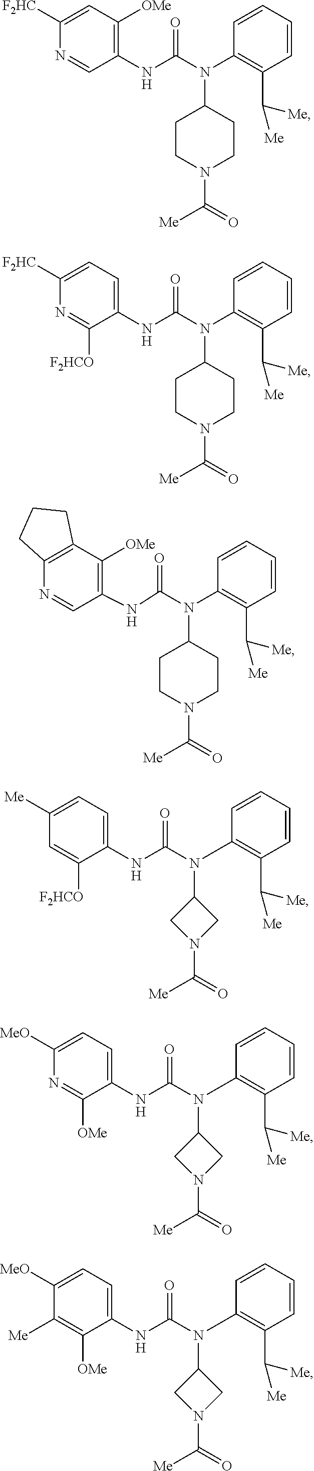 [SMILES: COc1cc(C(F)F)ncc1NC(=O)N(c1ccccc1C(C)C)C1CCN(C(C)=O)CC1 ; CC(=O)N1CCC(N(C(=O)Nc2ccc(C(F)F)nc2OC(F)F)c2ccccc2C(C)C)CC1 ; COc1c(NC(=O)N(c2ccccc2C(C)C)C2CCN(C(C)=O)CC2)cnc2c1CCC2 ; CC(=O)N1CC(N(C(=O)Nc2ccc(C)cc2OC(F)F)c2ccccc2C(C)C)C1 ; COc1ccc(NC(=O)N(c2ccccc2C(C)C)C2CN(C(C)=O)C2)c(OC)n1 ; COc1ccc(NC(=O)N(c2ccccc2C(C)C)C2CN(C(C)=O)C2)c(OC)c1C]

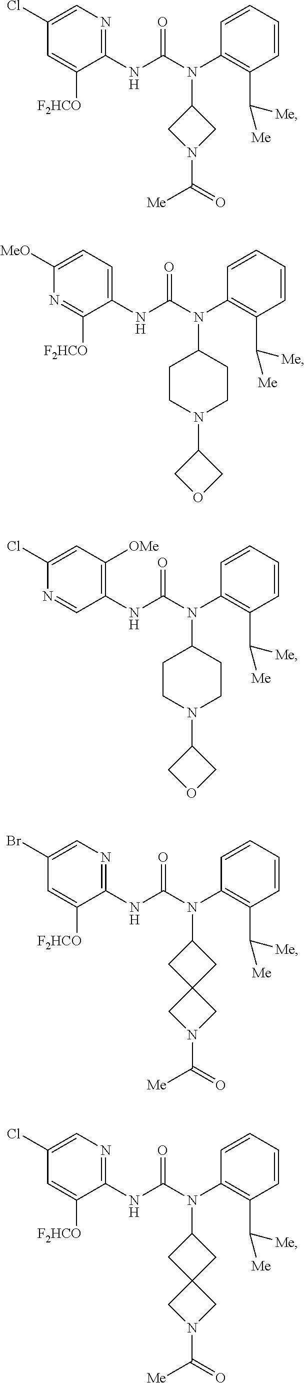 [SMILES: CC(=O)N1CC(N(C(=O)Nc2ncc(Cl)cc2OC(F)F)c2ccccc2C(C)C)C1 ; COc1ccc(NC(=O)N(c2ccccc2C(C)C)C2CCN(C3COC3)CC2)c(OC(F)F)n1 ; COc1cc(Cl)ncc1NC(=O)N(c1ccccc1C(C)C)C1CCN(C2COC2)CC1 ; CC(=O)N1CC2(CC(N(C(=O)Nc3ncc(Br)cc3OC(F)F)c3ccccc3C(C)C)C2)C1 ; CC(=O)N1CC2(CC(N(C(=O)Nc3ncc(Cl)cc3OC(F)F)c3ccccc3C(C)C)C2)C1]

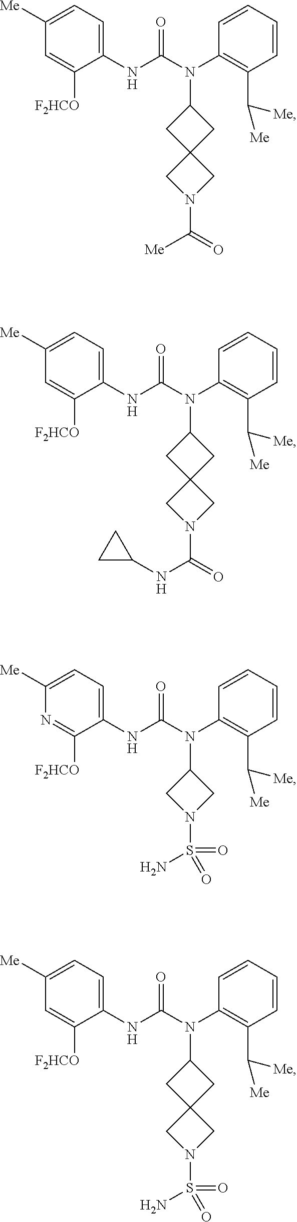 [SMILES: CC(=O)N1CC2(CC(N(C(=O)Nc3ccc(C)cc3OC(F)F)c3ccccc3C(C)C)C2)C1 ; Cc1ccc(NC(=O)N(c2ccccc2C(C)C)C2CC3(C2)CN(C(=O)NC2CC2)C3)c(OC(F)F)c1 ; Cc1ccc(NC(=O)N(c2ccccc2C(C)C)C2CN(S(N)(=O)=O)C2)c(OC(F)F)n1 ; Cc1ccc(NC(=O)N(c2ccccc2C(C)C)C2CC3(C2)CN(S(N)(=O)=O)C3)c(OC(F)F)c1]

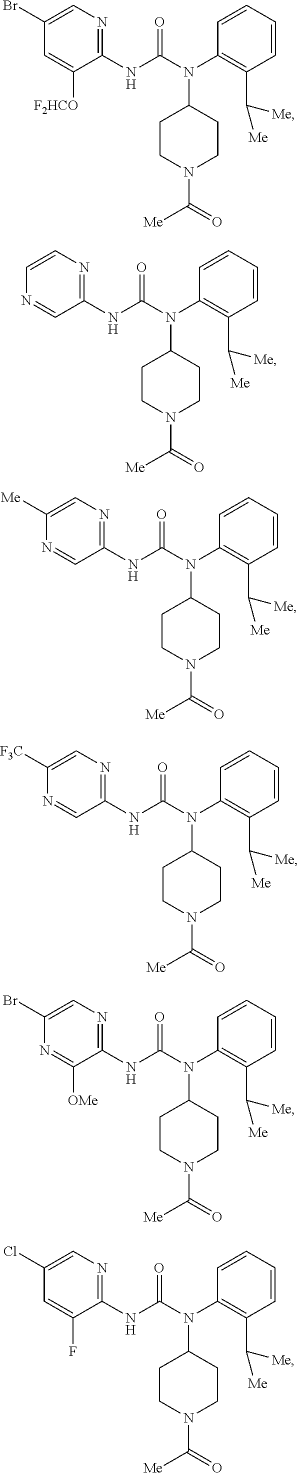

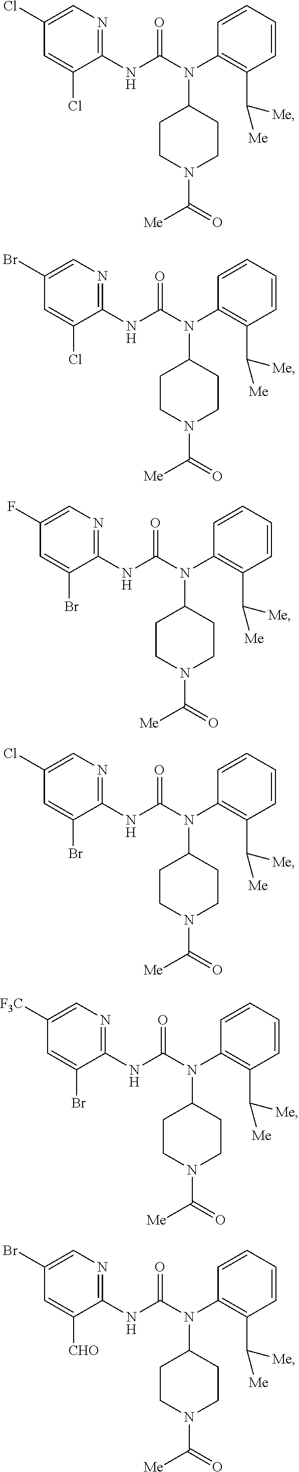

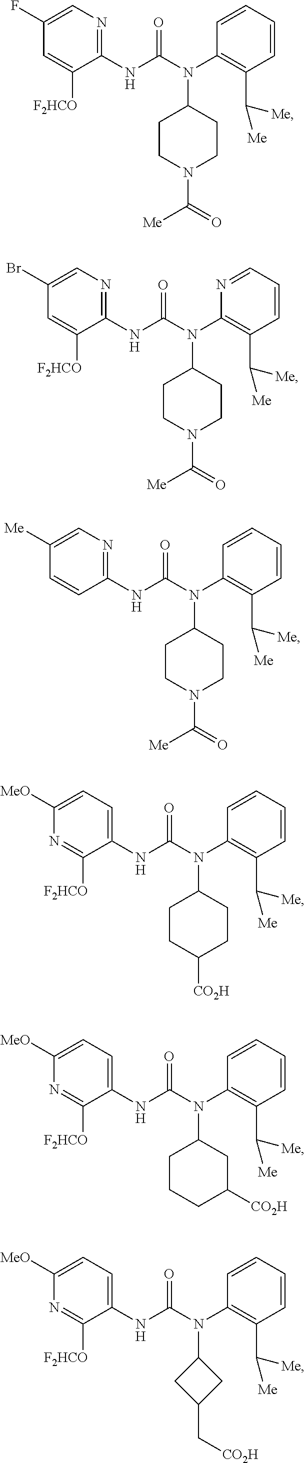 [SMILES: CC(=O)N1CCC(N(C(=O)Nc2ncc(F)cc2OC(F)F)c2ccccc2C(C)C)CC1 ; CC(=O)N1CCC(N(C(=O)Nc2ncc(Br)cc2OC(F)F)c2ncccc2C(C)C)CC1 ; CC(=O)N1CCC(N(C(=O)Nc2ccc(C)cn2)c2ccccc2C(C)C)CC1 ; COc1ccc(NC(=O)N(c2ccccc2C(C)C)C2CCC(C(=O)O)CC2)c(OC(F)F)n1 ; COc1ccc(NC(=O)N(c2ccccc2C(C)C)C2CCCC(C(=O)O)C2)c(OC(F)F)n1 ; COc1ccc(NC(=O)N(c2ccccc2C(C)C)C2CC(CC(=O)O)C2)c(OC(F)F)n1]

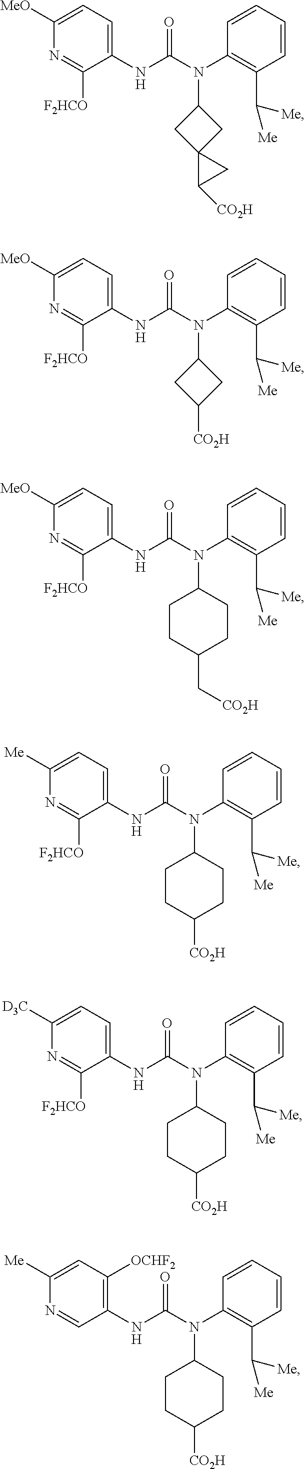 [SMILES: COc1ccc(NC(=O)N(c2ccccc2C(C)C)C2CC3(C2)CC3C(=O)O)c(OC(F)F)n1 ; COc1ccc(NC(=O)N(c2ccccc2C(C)C)C2CC(C(=O)O)C2)c(OC(F)F)n1 ; COc1ccc(NC(=O)N(c2ccccc2C(C)C)C2CCC(CC(=O)O)CC2)c(OC(F)F)n1 ; Cc1ccc(NC(=O)N(c2ccccc2C(C)C)C2CCC(C(=O)O)CC2)c(OC(F)F)n1 ; [2H]C([2H])([2H])c1ccc(NC(=O)N(c2ccccc2C(C)C)C2CCC(C(=O)O)CC2)c(OC(F)F)n1 ; Cc1cc(OC(F)F)c(NC(=O)N(c2ccccc2C(C)C)C2CCC(C(=O)O)CC2)cn1]

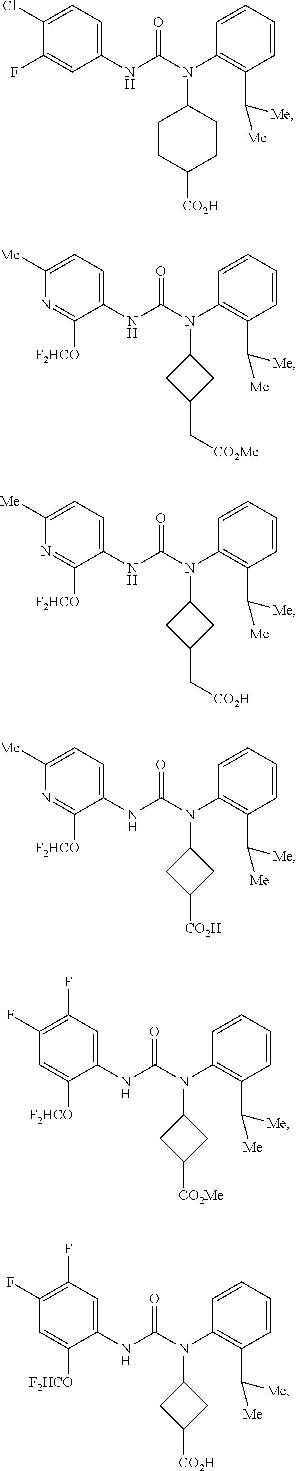 [SMILES: CC(C)c1ccccc1N(C(=O)Nc1ccc(Cl)c(F)c1)C1CCC(C(=O)O)CC1 ; COC(=O)CC1CC(N(C(=O)Nc2ccc(C)nc2OC(F)F)c2ccccc2C(C)C)C1 ; Cc1ccc(NC(=O)N(c2ccccc2C(C)C)C2CC(CC(=O)O)C2)c(OC(F)F)n1 ; Cc1ccc(NC(=O)N(c2ccccc2C(C)C)C2CC(C(=O)O)C2)c(OC(F)F)n1 ; COC(=O)C1CC(N(C(=O)Nc2cc(F)c(F)cc2OC(F)F)c2ccccc2C(C)C)C1 ; CC(C)c1ccccc1N(C(=O)Nc1cc(F)c(F)cc1OC(F)F)C1CC(C(=O)O)C1]

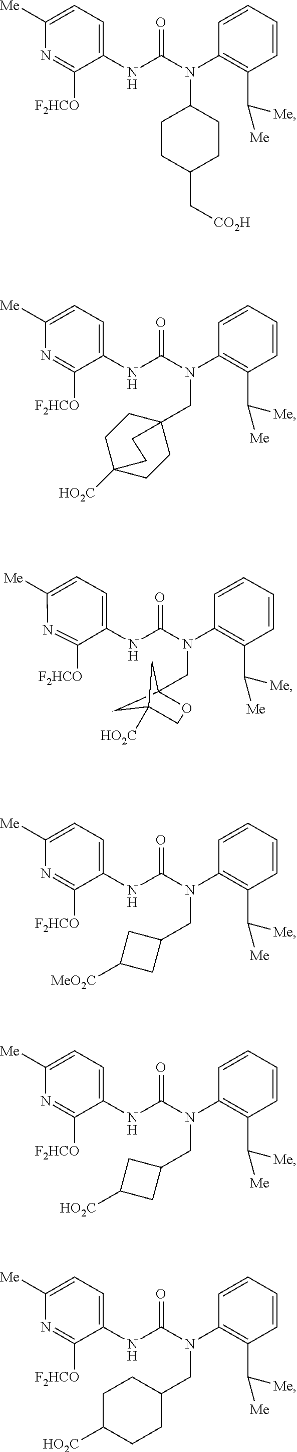 [SMILES: Cc1ccc(NC(=O)N(c2ccccc2C(C)C)C2CCC(CC(=O)O)CC2)c(OC(F)F)n1 ; Cc1ccc(NC(=O)N(CC23CCC(C(=O)O)(CC2)CC3)c2ccccc2C(C)C)c(OC(F)F)n1 ; Cc1ccc(NC(=O)N(CC23CC(C(=O)O)(CO2)C3)c2ccccc2C(C)C)c(OC(F)F)n1 ; COC(=O)C1CC(CN(C(=O)Nc2ccc(C)nc2OC(F)F)c2ccccc2C(C)C)C1 ; Cc1ccc(NC(=O)N(CC2CC(C(=O)O)C2)c2ccccc2C(C)C)c(OC(F)F)n1 ; Cc1ccc(NC(=O)N(CC2CCC(C(=O)O)CC2)c2ccccc2C(C)C)c(OC(F)F)n1]

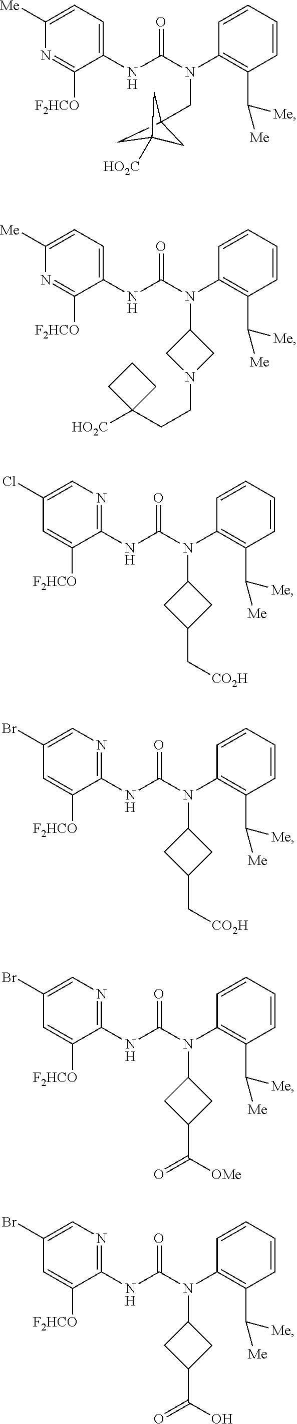 [SMILES: Cc1ccc(NC(=O)N(CC23CC(C(=O)O)(C2)C3)c2ccccc2C(C)C)c(OC(F)F)n1 ; Cc1ccc(NC(=O)N(c2ccccc2C(C)C)C2CN(CCC3(C(=O)O)CCC3)C2)c(OC(F)F)n1 ; CC(C)c1ccccc1N(C(=O)Nc1ncc(Cl)cc1OC(F)F)C1CC(CC(=O)O)C1 ; CC(C)c1ccccc1N(C(=O)Nc1ncc(Br)cc1OC(F)F)C1CC(CC(=O)O)C1 ; COC(=O)C1CC(N(C(=O)Nc2ncc(Br)cc2OC(F)F)c2ccccc2C(C)C)C1 ; CC(C)c1ccccc1N(C(=O)Nc1ncc(Br)cc1OC(F)F)C1CC(C(=O)O)C1]

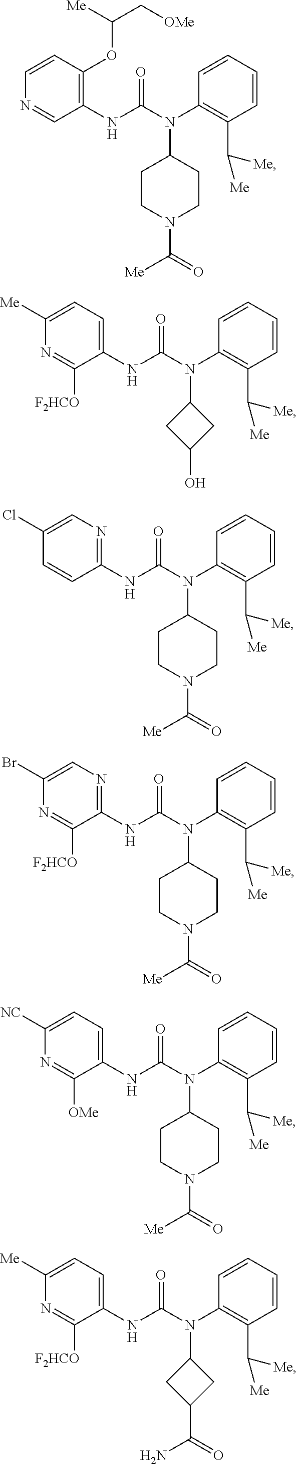

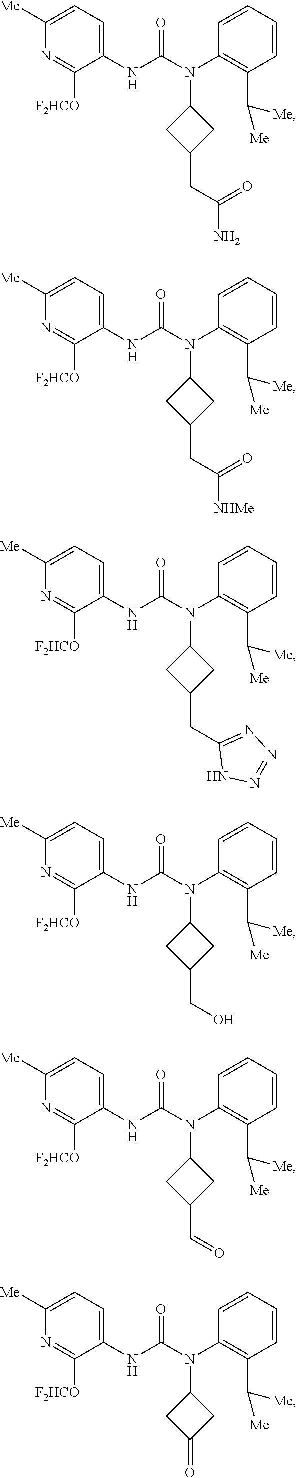

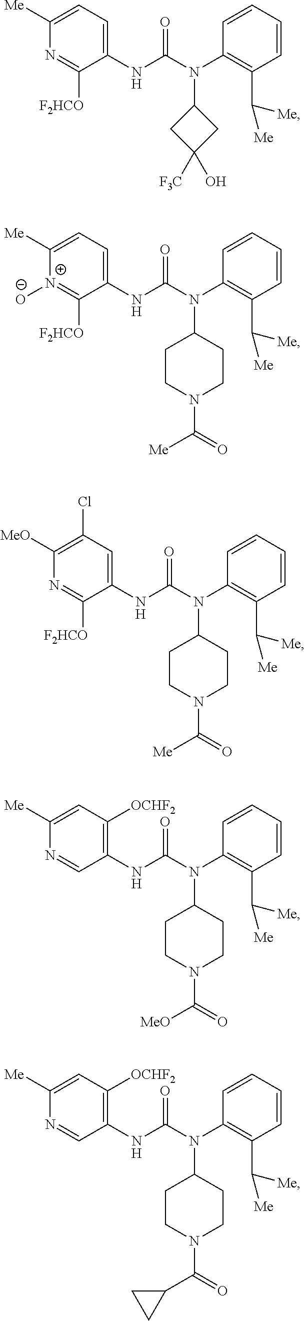

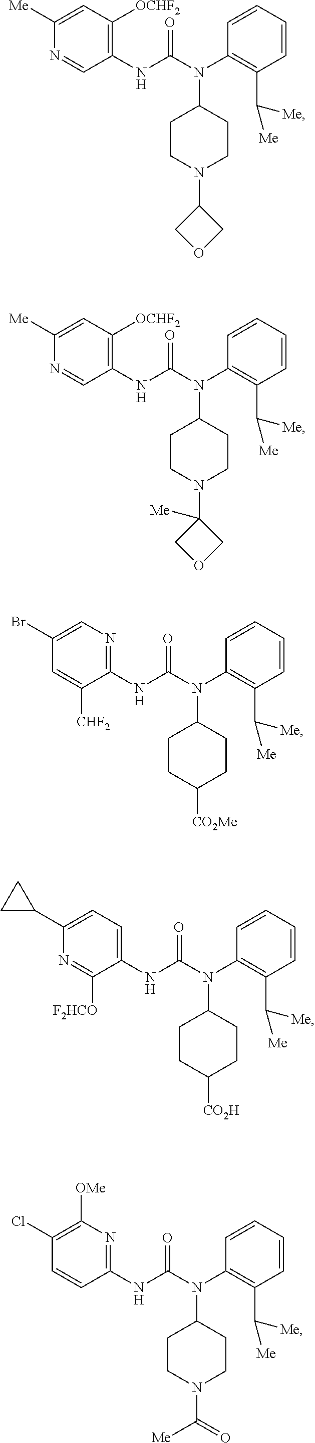 [SMILES: Cc1cc(OC(F)F)c(NC(=O)N(c2ccccc2C(C)C)C2CCN(C3COC3)CC2)cn1 ; Cc1cc(OC(F)F)c(NC(=O)N(c2ccccc2C(C)C)C2CCN(C3(C)COC3)CC2)cn1 ; COC(=O)C1CCC(N(C(=O)Nc2ncc(Br)cc2C(F)F)c2ccccc2C(C)C)CC1 ; CC(C)c1ccccc1N(C(=O)Nc1ccc(C2CC2)nc1OC(F)F)C1CCC(C(=O)O)CC1 ; COc1nc(NC(=O)N(c2ccccc2C(C)C)C2CCN(C(C)=O)CC2)ccc1Cl]

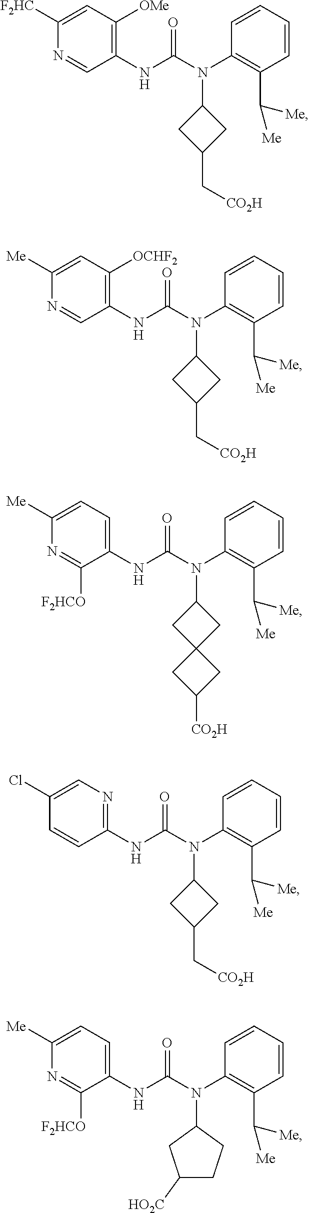 [SMILES: COc1cc(C(F)F)ncc1NC(=O)N(c1ccccc1C(C)C)C1CC(CC(=O)O)C1 ; Cc1cc(OC(F)F)c(NC(=O)N(c2ccccc2C(C)C)C2CC(CC(=O)O)C2)cn1 ; Cc1ccc(NC(=O)N(c2ccccc2C(C)C)C2CC3(CC(C(=O)O)C3)C2)c(OC(F)F)n1 ; CC(C)c1ccccc1N(C(=O)Nc1ccc(Cl)cn1)C1CC(CC(=O)O)C1 ; Cc1ccc(NC(=O)N(c2ccccc2C(C)C)C2CCC(C(=O)O)C2)c(OC(F)F)n1]

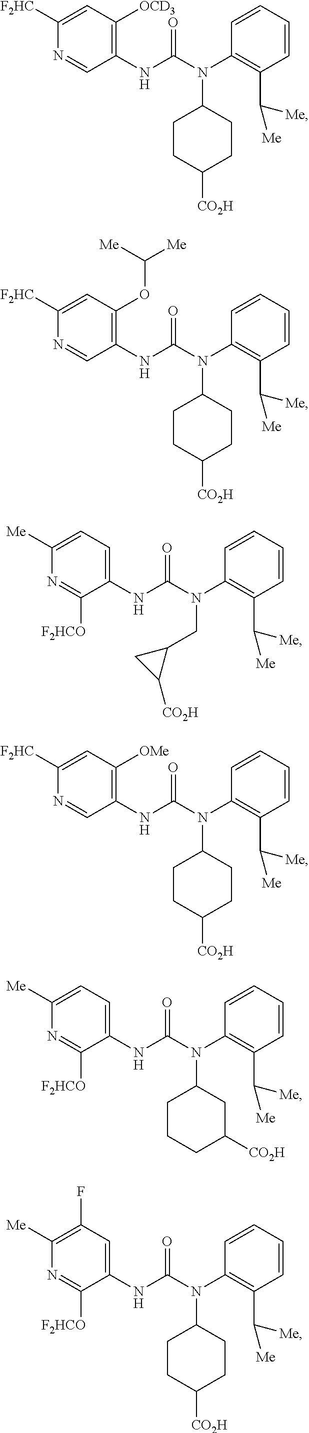

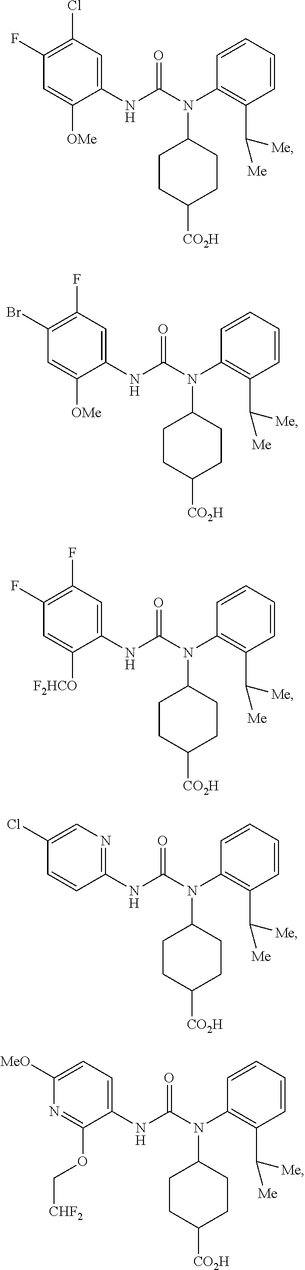 [SMILES: COc1cc(F)c(Cl)cc1NC(=O)N(c1ccccc1C(C)C)C1CCC(C(=O)O)CC1 ; COc1cc(Br)c(F)cc1NC(=O)N(c1ccccc1C(C)C)C1CCC(C(=O)O)CC1 ; CC(C)c1ccccc1N(C(=O)Nc1cc(F)c(F)cc1OC(F)F)C1CCC(C(=O)O)CC1 ; CC(C)c1ccccc1N(C(=O)Nc1ccc(Cl)cn1)C1CCC(C(=O)O)CC1 ; COc1ccc(NC(=O)N(c2ccccc2C(C)C)C2CCC(C(=O)O)CC2)c(OCC(F)F)n1]

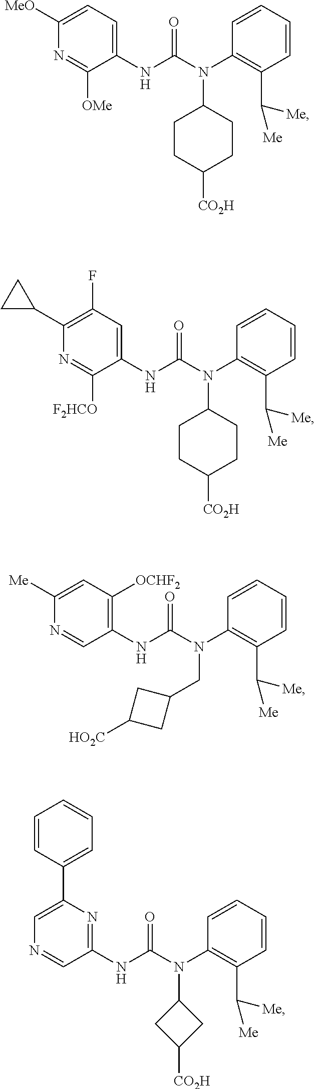 [SMILES: COc1ccc(NC(=O)N(c2ccccc2C(C)C)C2CCC(C(=O)O)CC2)c(OC)n1 ; CC(C)c1ccccc1N(C(=O)Nc1cc(F)c(C2CC2)nc1OC(F)F)C1CCC(C(=O)O)CC1 ; Cc1cc(OC(F)F)c(NC(=O)N(CC2CC(C(=O)O)C2)c2ccccc2C(C)C)cn1 ; CC(C)c1ccccc1N(C(=O)Nc1cncc(-c2ccccc2)n1)C1CC(C(=O)O)C1]

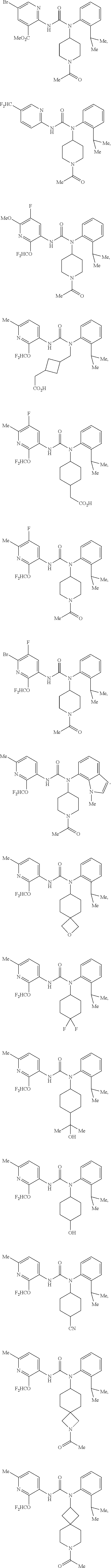

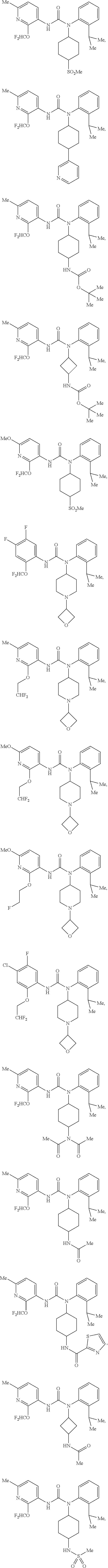

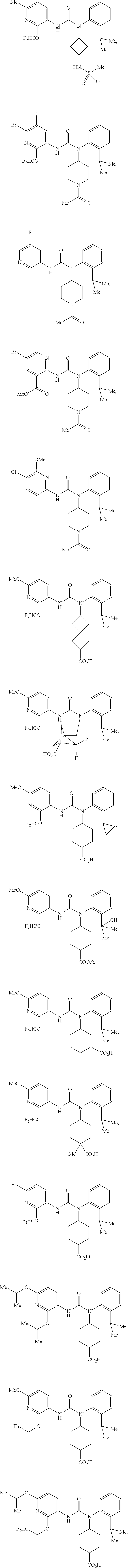

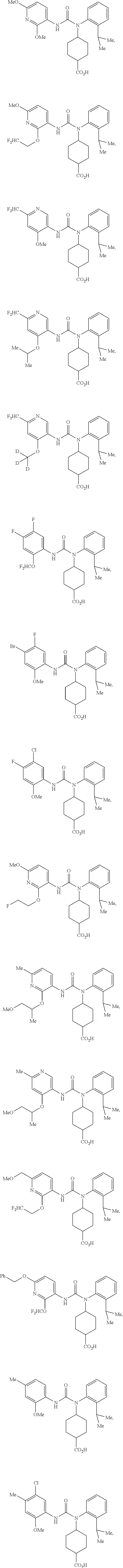

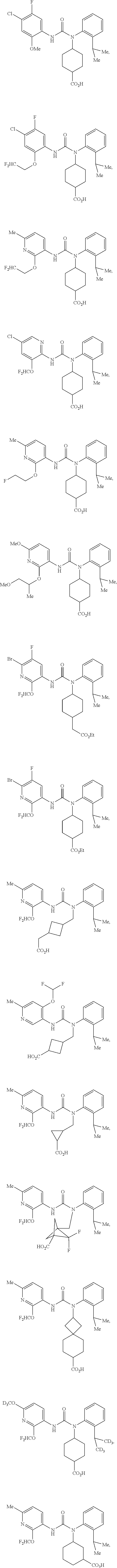

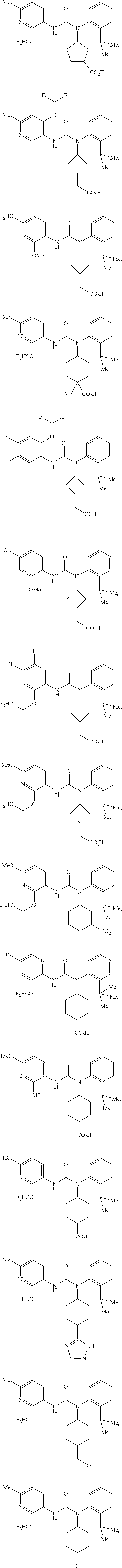

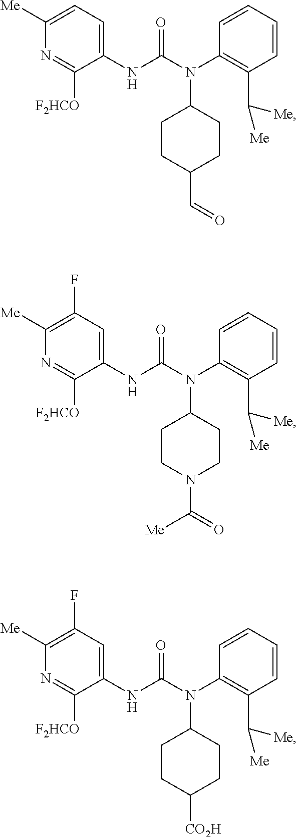 [SMILES: Cc1ccc(NC(=O)N(c2ccccc2C(C)C)C2CCC(C=O)CC2)c(OC(F)F)n1 ; CC(=O)N1CCC(N(C(=O)Nc2cc(F)c(C)nc2OC(F)F)c2ccccc2C(C)C)CC1 ; Cc1nc(OC(F)F)c(NC(=O)N(c2ccccc2C(C)C)C2CCC(C(=O)O)CC2)cc1F]

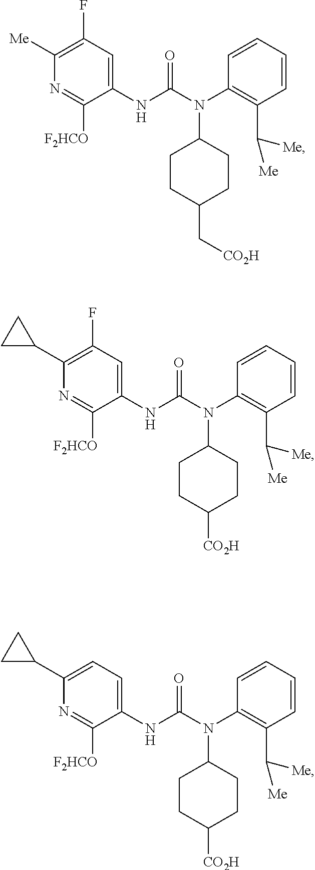 [SMILES: Cc1nc(OC(F)F)c(NC(=O)N(c2ccccc2C(C)C)C2CCC(CC(=O)O)CC2)cc1F ; CC(C)c1ccccc1N(C(=O)Nc1cc(F)c(C2CC2)nc1OC(F)F)C1CCC(C(=O)O)CC1 ; CC(C)c1ccccc1N(C(=O)Nc1ccc(C2CC2)nc1OC(F)F)C1CCC(C(=O)O)CC1]

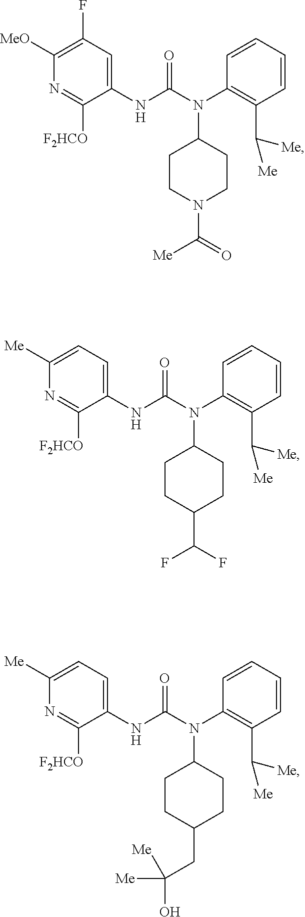 [SMILES: COc1nc(OC(F)F)c(NC(=O)N(c2ccccc2C(C)C)C2CCN(C(C)=O)CC2)cc1F ; Cc1ccc(NC(=O)N(c2ccccc2C(C)C)C2CCC(C(F)F)CC2)c(OC(F)F)n1 ; Cc1ccc(NC(=O)N(c2ccccc2C(C)C)C2CCC(CC(C)(C)O)CC2)c(OC(F)F)n1]

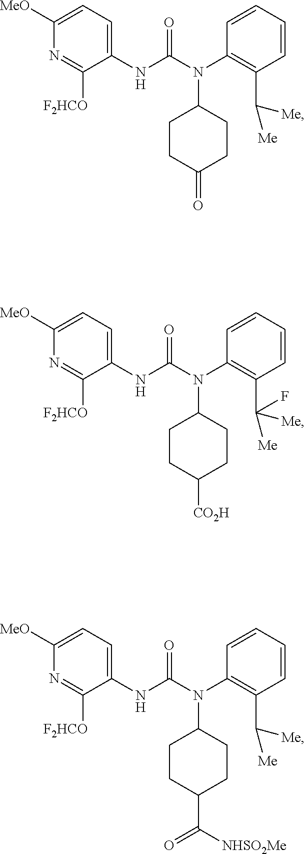

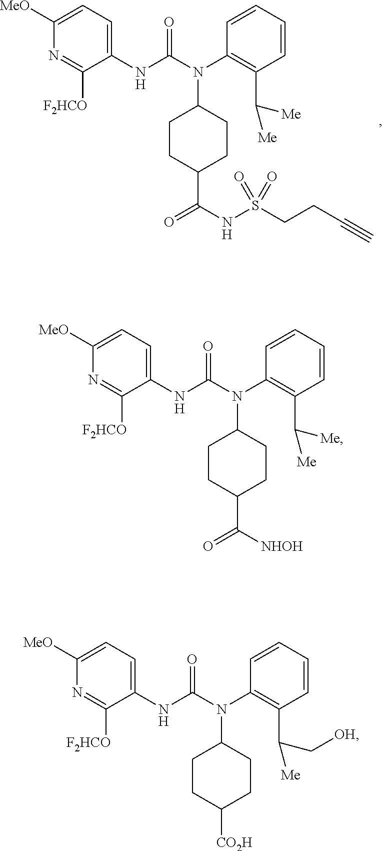

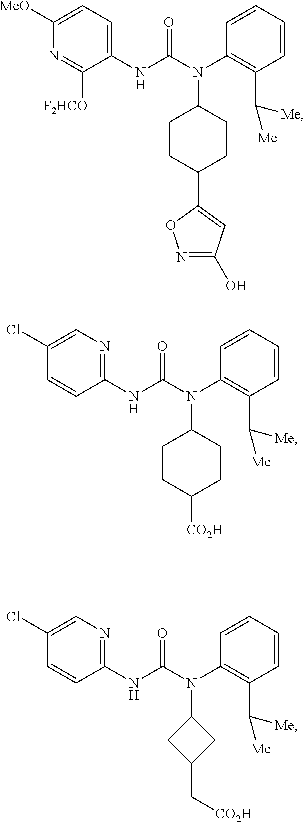 [SMILES: COc1ccc(NC(=O)N(c2ccccc2C(C)C)C2CCC(c3cc(O)no3)CC2)c(OC(F)F)n1 ; CC(C)c1ccccc1N(C(=O)Nc1ccc(Cl)cn1)C1CCC(C(=O)O)CC1 ; CC(C)c1ccccc1N(C(=O)Nc1ccc(Cl)cn1)C1CC(CC(=O)O)C1]

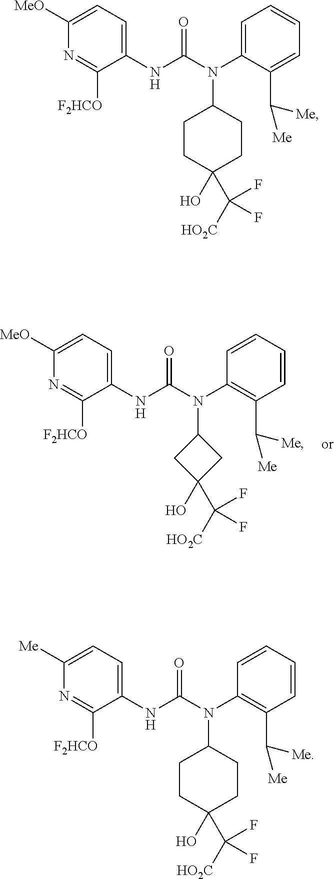 [SMILES: COc1ccc(NC(=O)N(c2ccccc2C(C)C)C2CCC(O)(C(F)(F)C(=O)O)CC2)c(OC(F)F)n1 ; COc1ccc(NC(=O)N(c2ccccc2C(C)C)C2CC(O)(C(F)(F)C(=O)O)C2)c(OC(F)F)n1 ; Cc1ccc(NC(=O)N(c2ccccc2C(C)C)C2CCC(O)(C(F)(F)C(=O)O)CC2)c(OC(F)F)n1]

41. A pharmaceutical composition comprising the compound of claim 1, or a pharmaceutically acceptable salt or solvate thereof, and a pharmaceutically acceptable excipient.

42. A method of treating a neurodegenerative disorder in a subject in need thereof, said method comprising administering to the subject in need thereof a therapeutically effective amount of the compound of claim 1, or a pharmaceutically acceptable salt or solvate thereof.

43. A method of treating an inflammatory disease in a subject in need thereof, said method comprising administering to the subject in need thereof a therapeutically effective amount of the compound of claim 1, or a pharmaceutically acceptable salt or solvate thereof.

44. (canceled)

45. (canceled)

46. A method of treating a demyelinating disease in a subject in need thereof, said method comprising administering to the subject in need thereof a therapeutically effective amount of the compound of claim 1, or a pharmaceutically acceptable salt or solvate thereof.

47.-49. (canceled)

50. A method of treating a fibrotic disease in a subject in need thereof, said method comprising administering to the subject in need thereof a therapeutically effective amount of the compound of claim 1, or a pharmaceutically acceptable salt or solvate thereof.

51. (canceled)

52. (canceled)

53. A method of treating cancer in a subject in need thereof, said method comprising administering to the subject in need thereof a therapeutically effective amount of the compound of claim 1, or a pharmaceutically acceptable salt or solvate thereof.

54. (canceled)

55. (canceled)

56. A method of modulating LPAR1 activity in a subject, said method comprising administering to the subject the compound of claim 1, or a pharmaceutically acceptable salt or solvate thereof.
